(19) United States
(12) Patent Application Publication
(10) Pub. No.: US 2026/0241049 A1
(43) Pub. Date: Aug. 20, 2026

(54) Radiopharmaceutical Compositions Targeting Glypican-3 and Uses Thereof

(71) Applicant: RayzeBio, Inc., San Diego, CA (US)

(72) Inventors: Alain Noncovich, San Diego, CA (US); Abhijit Bhat, Carlsbad, CA (US); Rongjun He, San Diego, CA (US); Takeru Ehara, Kanagawa (JP); Rie Komura, Kanagawa (JP); Yoshihide Mizukoshi, Kanagawa (JP); Kouki Morimoto, Kanagawa (JP); Masatoshi Takuwa, Kanagawa (JP); Hayato Yanagida, Kanagawa (JP); Masaki Ohuchi, Kanagawa (JP); Takayuki Nagasawa, Kanagawa (JP)

(73) Assignee: RayzeBio, Inc., San Diego, CA (US)

(21) Appl. No.: 19/395,559

(22) Filed: Nov. 20, 2025

Related U.S. Application Data
(63) Continuation of application No. PCT/US24/32857, filed on Jun. 6, 2024.
(60) Provisional application No. 63/506,721, filed on Jun. 7, 2023.

(51) Int. Cl. A61K 51/08

(52) U.S. Cl. CPC A61K 51/088

(57) ABSTRACT

Provided herein are radiopharmaceuticals and conjugates, and compositions thereof targeting GPC3 and uses thereof. In one aspect, provided herein are conjugates that comprise a peptide with avidity to GPC3 and a metal chelator configured to bind with a radionuclide. The conjugates described herein can further comprise a linker connecting the chelator and the peptide. The conjugates described herein can further comprise a radionuclide. In one aspect, provided herein are radiopharmaceuticals that comprise a peptide and a metal chelator bound to a radionuclide. The conjugates described herein can further comprise a linker connecting the chelator and the peptide. Further provided herein are methods of treating cancer by administering the described radiopharmaceuticals, conjugates, compositions thereof.

### CROSS REFERENCE TO RELATED APPLICATIONS

This application is a continuation of International Patent Application No. PCT/US2024/032857, filed on Jun. 6, 2024, which claims the benefit of U.S. Provisional Application No. 63/506,721, filed on Jun. 7, 2023, which is incorporated herein by reference in its entirety.

### JOINT RESEARCH AGREEMENT

Subject matter disclosed herein was developed, and the claimed invention was made by, or on behalf of, one or more parties to a Joint Research Agreement (JRA), within the meaning of 35 U.S.C. § 100(h) and 37 C.F.R. § 1.9(e), that was in effect on or before the effective filing date of the claimed invention. Said one or more parties to the JRA consist of PeptiDream, Inc. (Kanagawa, Japan) and RayzeBio, Inc. (San Diego, CA, U.S.A.). The claimed invention was made as a result of activities undertaken within the scope of said Joint Research Agreement.

### SEQUENCE LISTING

The present application contains a Sequence Listing which has been submitted electronically in .xml format as part of the specification and is incorporated herein by reference in its entirety. Said XML file, created on Jun. 5, 2024, is named 59541-732_601_SL.xml, and is 689,382 bytes in size.

### TECHNICAL FIELD

In one aspect, the present disclosure relates to radiopharmaceutical conjugates comprising peptides that bind to Glypican-3 (GPC3) and to compositions comprising such radiopharmaceutical conjugates and uses thereof. In one aspect, the present disclosure relates to the use of said radiopharmaceutical conjugates in preventing, suppressing or treating a disease or disorder characterized by either overexpression or decreased expression of GPC3 in diseased tissue, such as in a tumor when GPC3 is overexpressed.

### BACKGROUND

Glypican-3 (GPC3) is a heparan sulfate (HS) glycoprotein, belonging to the sulfate heparan proteoglycan family, and which is anchored on the cell membrane surface by phosphatidylinositol (GPI) anchor. The GPC3 core protein comprises 580 amino acids, with a molecular weight of about 70 kDa. It is cut by furin (Furin), generating a 40 kDa N-terminal subunit and a 30 kDa C-terminal subunit, connected to each other by a disulfide bond. GPC3's two HS side chain is combined at the position close to the C end (Takahiro Nishida, Hiroaki Kataoka. Glypican 3-Targeted Therapy in Hepatocellular Carcinoma, Cancer 2019; 11 (9): 1339).

GPC3 can play an important role in the cell proliferation of embryo layer tissue. Deletion of GPC3 gene can cause excessive growth syndrome, namely Simpson-Golabi-Behmel syndrome (SGBS). GPC3 can be clearly expressed throughout the entire fetal stage, and after birth to adult stage, except for placental, breast, mesodermal, ovarian, lung and kidney tissue with weak expression, other normal tissues have no obvious expression.

Abnormal GPC3 expression has been found in multiple tumour tissues of adult, such as hepatocellular carcinoma (HCC), lung squamous carcinoma, gastric cancer, ovarian cancer and so on. Especially highly expressed in HCC cells, GPC3 improves autocrine/paracrine canonical Wnt signal transmission, and promotes growth and invasion of HCC cells (Capurro M I, Xiang Y- Y, Lobe C, Filmus J. Glypican-3 promotes the growth of hepatocellular carcinoma by-stimulating canonical Wnt signaling; Cancer Res 2005, 65(14): 6245-54.). Immunohistochemical staining detection shows that about 70% of HCC patient tumor tissue exhibits high GPC3 protein expression (Capurro M, Wanless I R, Sherman M I, et al. Glypican-3: a novel serum and histochemical marker for hepatocellular carcinoma; Gastroenterology 2003, 125(1):89-97). Thus, GPC3 is considered as a candidate target for tumor treatment.

Codrituzumab (also known as GC33 antibody) is a recombinant humanized monoclonal antibody developed in Japan by Chugai Pharmaceutical Co., which binds to the region of GPC3 protein proximal membrane end. GC33 antibody targets GPC3 positive HCC cells, and can generate antibody dependent cell toxicity (ADCC). In phase I clinical trials, Codrituzumab shows good immune tolerance. The HCC patient can generate an anti-tumour effect (Ikeda M, Ohkawa S, Okusaka T. et al. Japanese phase I study of GC33, a-antibody against glypican-3 for advanced hepatocellular carcinoma. Cancer Sci. 2014, 105, 455-462). However, in the phase II clinical trial with 185 late-stage liver cancer patients, the therapeutic efficacy of Codrituzumab was not impressive compared with the control group.

Therefore, novel GPC3-binding peptides and compositions comprising the GPC3-binding peptide are both useful and desired.

### SUMMARY

In one aspect, disclosed herein is a radiopharmaceutical or a pharmaceutically acceptable salt thereof, the radiopharmaceutical comprising:
(a) a peptide of Formula (I-1),

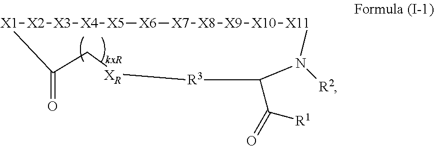

wherein:
   R¹ is —NH₂ or —OH;
   R² is C₁₋₃ alkyl;
   R³ is C₁ alkylene, optionally substituted with one or more R⁴;
      each R⁴ is independently C₁₋₃ alkyl or C₃₋₆ cycloalkyl;
   kxR is 1, 2, 3, 4, 5, or 6;
   X_{R} is —S—, —CH— or —O—;
   X1 is any amino acid;
   X2 is any amino acid:
   X3 is any amino acid;
   X4 is any amino acid;
   X5 is an amino acid comprising an aromatic ring, or cycloalkyl or heterocycloalkyl group, or X5 is a peptoid;
   X6 is a hydrophobic amino acid, a hydrophilic amino acid, or a polar amino acid wherein the polar amino acid has a substituted side chain;
   X7 is a hydrophobic amino acid comprising a C₁-C₅ alkyl, cycloalkyl, or heterocycloalkyl, wherein the alkyl, cycloalkyl, and heterocycloalkyl are each independently, optionally substituted;
   X8 is A, I, L, V, Y, or F. or a variant thereof:
   X9 is an N-alkylated amino acid comprising an aromatic ring;
   X10 is C or A, or an N-alkylated variant thereof, or a D-amino acid; and
   X11 is an amino acid comprising an aromatic ring; and
(b) a metal chelator bound to a radionuclide, wherein the metal chelator is covalently attached to the peptide.

In some embodiments, the radiopharmaceutical is selected from Table 3A or Table 3B.

In one aspect, disclosed herein is a conjugate or a pharmaceutically acceptable salt thereof, the conjugate comprising:
(a)a peptide of Formula (I-1),

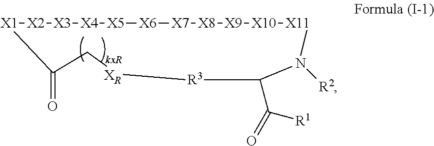

wherein:
   R¹ is —NH₂ or —OH;
   R² is C₁₋₃ alkyl;
   R³ is C₁ alkylene, optionally substituted with one or more R⁴;
      each R⁴ is independently C₁₋₃ alkyl or C₃₋₆ cycloalkyl;
   kxR is 1, 2, 3, 4, 5, or 6;
   X_{R} is —S—, —CH₂—, or —O—;
   X1 is any amino acid;
   X2 is any amino acid:
   X3 is any amino acid;
   X4 is any amino acid;
   X5 is an amino acid comprising an aromatic ring, or cycloalkyl or heterocycloalkyl group, or X5 is a peptoid;
   X6 is a hydrophobic amino acid, a hydrophilic amino acid, or a polar amino acid wherein the polar amino acid has a substituted side chain;
   X7 is a hydrophobic amino acid comprising a C₁-C₈ alkyl, cycloalkyl, or heterocycloalkyl, wherein the alkyl, cycloalkyl, and heterocycloalkyl are each independently, optionally substituted;
   X8 is A, I, L, V, Y, or F. or a variant thereof:
   X9 is an N-alkylated amino acid comprising an aromatic ring;
   X10 is C or A, or an N-alkylated variant thereof, or a D-amino acid; and
   X11 is an amino acid comprising an aromatic ring; and
(b) a metal chelator configured to bind with a radionuclide, wherein the metal chelator is covalently attached to the peptide.

In some embodiments, the conjugate is selected from Table 9.

In one aspect, the present disclosure relates to a radiopharmaceutical conjugate comprising:
(a) a peptide that has avidity for Glypican 3 (GPC3), or a pharmaceutically acceptable salt thereof, wherein the peptide comprises an amino acid sequence including deletion, substitution, and/or addition of one or several (e.g., 1-6) amino acids in the amino acid sequence of SEQ ID NO: 1:

(SEQ ID NO: 1)
MeK-MeI-D-MeQ-F4COO-I-I-Y-MeNal27N-G-3Py6Ph-MeC,

wherein the peptide consists of 10 to 12 amino acid residues; and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide, wherein the metal chelator is bound to the peptide. In some embodiments, the radiopharmaceutical conjugate comprises a radionuclide bound to the metal chelator. In some embodiments, the radiopharmaceutical conjugate comprises a covalently bound radionuclide. In some embodiments, the peptide is a cyclic peptide. In some embodiments, the peptide is a monocyclic peptide.

In one aspect, the present disclosure relates to a radiopharmaceutical conjugate comprising:
(a) a peptide that has avidity for Glypican 3 (GPC3), or a pharmaceutically acceptable salt thereof, wherein the peptide comprises an amino acid sequence of Formula (I),

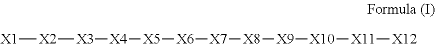

wherein,
   X1 is any amino acid;
   X2 is any amino acid;
   X3 is any amino acid:
   X4 is any amino acid;
   X5 is an amino acid comprising an aromatic ring (e.g., W, F, Y, or a variant thereof), cycloalkyl, or heterocycloalkyl group, or X5 is a peptoid (e.g., Cha4cH, Cha4tH, A1mor, Atp. Cha4cOMe);
   X6 is a hydrophobic amino acid, a hydrophilic amino acid, or a polar amino acid wherein the polar amino acid has a substituted side chain;
   X7 is a hydrophobic amino acid comprising a C₁-C₈ alkyl, cycloalkyl, or heterocycloalkyl, wherein the alkyl, cycloalkyl, and heterocycloalkyl are each independently, optionally substituted (e.g., X7 is I, Eva, alI, TMe, SMe, Gcpr, Gepe, Gthp, dMeS, TdMe, or Cbg);
   X8 is a, I, L, V, Y, or F. or a variant thereof;
   X9 is an N-alkylated amino acid comprising an aromatic ring:
   X10 is G, A. or a D-amino acid (e.g., da, ds, de, or dp);
   X11 is an amino acid comprising an aromatic ring (e.g., F, Y, or a variant thereof); and,
   X12 is N-alkylated cysteine (e.g., MeC); and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide, wherein the metal chelator is bound to the peptide. In some embodiments, the radiopharmaceutical conjugate comprises a covalently bound radionuclide. In some embodiments, the peptide is a monocyclic peptide. In some embodiments, the peptide has an amino acid sequence according to Formula (I), or a pharmaceutically acceptable salt thereof,

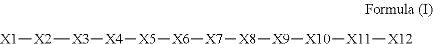

wherein,
   X1 is I, R, Cit, F4G, 4Py, 3Py, KCOpipzaa, V, Eva, Q, E, MeI, Ahp, F4COO, KCOpip4COO, MeQdMe, MeA, MeSMe, MeG, MeV, MeHseMe, Aib, MeT, alI, TMe, MeKCOpipzaa, MeQ, Hpr, MeTMe, MeDapCOpipzaa. MeK, MeKAc, MeK(de), MeK(H), MeK(df), or MeK(datb);
   X2 is I, K, Cit, F4G, 4Py, 3Py. KCOpipzetOH. V, KCOpipzaa, Eva, Q, E, S, Aip, F4COO, KCOpip4COO, MeQdMe, MeA, MeSMe, MeG, MeI, MeV, MeL, HseMe, MeY, Me3Py, MeIIseMe, MeKAc, alI, TMe MeKCOpipzaa, MeQ, Hpr, MeTMe, or McDapCOpipzaaa;
   X3 is D, Har, KCOpipzetOH, Cit, KCOmeglumine, KCOpipzaa, A4paa, Q, A, E, MeD, S, N, Hgl, F4COO. KCOpip4COO. KAc, Hgn, MeY, or DapCOpipzaa;
   X4 is D, Har. KCOpipzetOH. KCOmeghamine, KCOpipzaa, A4paa, Q, A, E, MeD, S, N, Hgl, F4COO, KCOpip4COO, dd, MeQ, MeQdMe, MeA, MeSMe, MeG, EtG, MeeG, CmG, CmpG, CrnG, CeG, CrpG, MeK, MeKAc, MeHgl, Hgn, MeDapCOpipzaa, MeKCOpipzaa, Medd, Cit, MeCit, MeN, MeS, MeE, MeY, W5N, or MeA4paa;
   X5 is Y, F3G, 3Py6COO,4Py2NH2, 3Py5COO, F3COO, 3Py6NHAc, F, F4C, F40Me, F4COO, Nal2, F3aao, F4aa, F4aao, 3Py6NHaa, 5Pdo, F3CON, F4F, F4OEt. F4Me, F4CON, F4CONPEG4Me, F3OMe, F3CON, Yac, YaeCOpipzaa, F4aaopipzaa, 4Pdo, 3Py6CON, Atp, Cha4cH, Cha4tH, Cha4cOMe, A1mor, or F4amCOpipzaa;
   X6 is 1, V, Eva, Chg, Tbg, A, L, Ahp, F4COO, Gcpr. Gcpe, alI, Cle, S3REt, TMe, Acpr, Cba, Gthp, NIeCOO, NleOH, P, Atb, Nva, Nle, N, DapAc, Abu, Nmm, Ndin, Ncit, Cit, SMe, HseMe, HseEt, HseiPr, dMeS, TdMe. Cbg, NyaOMe, SiPr, Spr, NleOMe, Sbu, Scbn, Scpe, AhpOMe, HseBu, Spent or Hsecpe;
   X7 is I, Eva, alI, TMe, SMe, Gcpr, Gcpe, Gthp, dMeS, TdMe, or Cbg;
   X8 is A, 1, L, V, Y. F40Me, F4COO, F40Et, F4u, F4Me, F4CONdMe, F4CON, F4 ms, F4CONPEG4Me, F34dOMe, F30Me, F3C, F3CON, F3CONdMe, 3Py6CON, Yae, YaeCOpipzaa, 5Inda, F3aao, F3aa, F4aao, F4aa, 3Py6Nhae, 3Py6NHAc, 3Py6OMe, F4aaopipzaa, or F4amCOpipzaa;
   X9 is MeNal2, MeNal27N. MeF34diox, MeF34dOMe, MeF4T, MeY, MeW1Me, MeW7N, MeF3C4Me, or MeF3Me4C;
   X10 is G, A. or a D-amino acid (e.g., da, ds, de, or dp);
   X11 is Bph, 3Py6Ph, F41Me4Pyz, F43Pyz, F44Pyz, F41Pyz, F41Me3Pyz, F41Et4Pyz, F41MeOe4Pyz, F41MeOp4Pyz, F44thp, F4Ac4pip, PhNva, PhNIe, Yph, Ybn, F4tb, F4oPr, or F4CONdMe; and
   X12 is MeC.

In one aspect, the present disclosure relates to a radiopharmaceutical conjugate comprising:
(a) a peptide having avidity for Glypican 3 (GPC3), or a pharmaceutically acceptable salt thereof, wherein the peptide has an amino acid sequence of Formula (I),

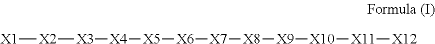

wherein,
   X1 is any amino acid;
   X2 is any amino acid;
   X3 is any amino acid;
   X4 is any amino acid;
   X5 is

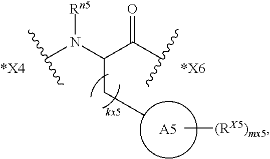

wherein:
   Rⁿ⁵ is hydrogen or C₁₋₃ alkyl:
   ring A5 is an aryl, heteroaryl, cycloalkyl, or heterocycloalkyl;
   each R^{X5} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂NR^{c}R^{d}, —S(═O)(═NR^{a})R^{a}, —N═S(═O)NR^{c}R_{d}, —NR^{c}S(═O)₂R^{a}, amidinyl, —NR^{a}C(═NH)NR^{c}R^{d}, —NR^{a}S(═O)₂NR^{c}R^{d}, —C(═O)R^{a}, —C(═)OR^{a}, —OC(═O)R^{d}, OC(═)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R_{d}, aryl, heteroaryl, cycloalkyl, heterocycloalkyl, -L^{X5}-heterocycloalkyl, -L^{X5}-cycloalkyl, -L^{X5}-aryl, or -L^{X5}-heteroaryl, wherein the alkyl, heteroalkyl, aryl, heteroaroyl, cycloalkyl, and heterocycloalkyl are optionally substituted with one or more R^{X5a}; or
   two R^{X5} are taken together to form ═O, ═S, or ═N(R^{a}):
   L^{X5} is C₁₋₆alkylene, C₁₋₆heteroalkylene, —O—, —S—, or —NR^{a}—, wherein the alkylene and heteroalkylene is optionally substituted with one or more R^{X5a};
   kx5 is 0, 1, 2, or 3;
   mnx5 is 0, 1, 2, 3, 4, or 5;
   *X4 represents the point of attachment to X4; and
   *X6 represents the point of attachment to X6;
X6 is

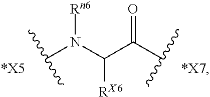

wherein;
   Rⁿ⁶ is hydrogen or C₁₋₃ alkyl;
   R^{X6} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₁₋₆alkynyl, aryl, heteroaryl, cycloalkyl, heterocycloalkyl, -L^{X6}-heterocycloalkyl, -L^{X6}-cycloalkyl, -L^{X6}-aryl, or -L^{X6}-heteroaryl, wherein each of the alkyl, heteroalkyl, alkenyl, alkynyl, aryl, heteroaryl, cycloalkyl, and heterocycloalkyl is optionally substituted with one or more R^{X6a}; or
   Rⁿ⁶ and R^{X6} are taken together with the intervening atoms to form a 5-to 6-membered heterocycloalkyl, which is optionally substituted with one or more R^{X6};
   L^{X6} is C₁₋₆alkylene, C₁₋₆heteroalkylene, —O—, —S—, or —NR^{a}—, wherein the alkylene and heteroalkylene is optionally substituted with one or more R^{X6a}.
   *X5 represents the point of attachment to X5; and
   *X7 represents the point of attachment to X7;
X7 is

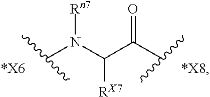

wherein:
   Rⁿ⁷ is hydrogen or C₁₋₆alkyl;
   R^{X7} is C₁₋₆alkyl, C₁₋₆haloalkyl, C₁₋₆hydroxyalkyl, C₁₋₆aminoalkyl, C-heteroalkyl, C₂₋₆alkenyl, C₂₋₆halkynyl, cycloalkyl, or heterocycloalkyl, wherein each of the alkyl, heteroalkyl, alkenyl, alkynyl, cycloalkyl, and heterocycloalkyl is optionally substituted with one or more R^{X7a};
   *X6 represents the point of attachment to X6; and
   *X8 represents the point of attachment to X8;
X8 is

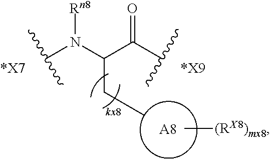

wherein;
   Rⁿ⁸ is hydrogen or C₁₋₃alkyl;
   ring A8 is an aryl or heteroaryl;
   each R^{X8} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X8}-heterocycloalkyl, -L^{X8}-cycloalkyl, -L^{X8}-aryl, or -L^{X8}-heteroaryl, wherein each of the alkyl, heteroalkyl, alkenyl, alkynyl, aryl, heteroaryl, cycloalkyl, and heterocycloalkyl is optionally substituted with one or more R^{X8a}; or
   two R^{X8} are taken together to form ═O, ═S, or ═N(R^{a});
   L^{X8} is C₁₋₅alkylene, C₁₋₆heteroalkylene, —O—, —S—, or —NR^{a}—, wherein the alkylene and heteroalkylene is optionally substituted with one or more R^{X8a};
   kx8 is 0, 1, 2, or 3;
   mx8 is 0, 1, 2, 3, 4, or 5;
   *X7 represents the point of attachment to X7; and
   *X9 represents the point of attachment to X9.
X9 is

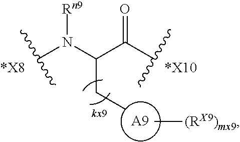

wherein:
   Rⁿ⁹ is hydrogen or C₁₋₃ alkyl;
   ring A9 is an aryl or heteroaryl;
   each R^{X9} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR², —SF₅, or —NR^{c}R^{d}, wherein each of the alkyl, heteroalkyl, alkenyl, and alkynyl is optionally substituted with one or more R^{X9a}; or
   two R^{X9} are taken together to form ═O, ═S, or ═N(R^{a});
   kx9 is 0, 1, 2, or 3;
   mx9 is 0, 1, 2, 3, 4, or 5;
   *X8 represents the point of attachment to X8, and
   *X10 represents the point of attachment to X10;
X10 is glycine or a D-amino acid (e.g., da, ds, de, or dp);
X11 is

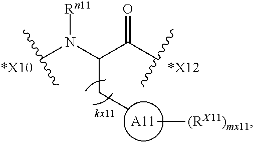

wherein:
   Rⁿ¹¹ is hydrogen or C₁₋₃alkyl;
   ring A11 is an aryl or heteroaryl;
   each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR³)R³, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{q}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR³C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X11}-heterocycloalkyl, -L^{X11}-cycloalkyl, -L^{X11}-aryl, or -L^{X11}-heteroaryl, wherein each of the alkyl, heteroalkyl, aryl, heteroaryl, cycloalkyl, and heterocycloalkyl is optionally substituted with one or more R^{X11a}; or
   two R^{X11} are taken together to form ═O, ═S, or ═N(R^{a});
   L^{X11} is C₁₋₆alkylene, C₁₋₆heteroalkylene, —O—, —S—, or —NR²—, wherein the alkylene and heteroalkylene is optionally substituted with one or more R^{X11a};
   kx11 is 0, 1, 2, 3, 4, or 5;
   mx11 is 0, 1, 2, 3, 4, or 5;
   *X10 represents the point of attachment to X10; and,
   *X12 represents the point of attachment to X12;
X12 is N-alkylated cysteine;
each of R^{X5a}, R^{X6a}, R^{X7a}, R^{X8a}, R^{X9a} and R^{X11a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(O)OR^{a}, —NR^{a}C(═O)NR^{c}R^{d}, —C(O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, and alkynyl is optionally substituted with one or more R^{c},
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e};
each R^{e} is independently halogen, —CN, —OH, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆haloalkyl, C₁-C₆hydroxyalkyl, C₁-C₆aminoalkyl, or C₁-C₆heteroalkyl; or two R^{e} are taken together to form ═O; and
each R^{c} and R^{d} are independently hydrogen, C₁-C₆alkyl, C₁-C₆haloalkyl, C₁-C₆hydroxyalkyl, C₁-C₅aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e}; and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide, wherein the metal chelator is bound to the peptide. In some embodiments, the radiopharmaceutical conjugate comprises a covalently bound radionuclide. In some embodiments, the peptide is a monocyclic peptide.

In some embodiments of the radiopharmaceutical conjugate, the peptide of Formula (I) has a structure of Formula (I-1):

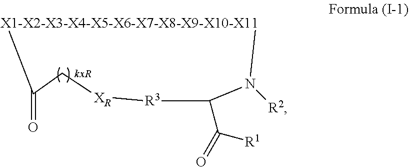

wherein,
   R¹ is selected from the group consisting of —NH₂ and —OH;
   R² is C₁₋₃ alkyl;
   R³ is C₁₋₃ alkylene, optionally substituted with one or more R⁴;
   each R⁴ is independently C₁₋₃ alkyl or C₃₋₆ cycloalkyl;
   kxR is 1, 2, 3, 4, 5, or 6;
   X_{R} is selected from the group consisting of —S—, —CH₂—, or —O—; and
wherein X1 to X11 have the definitions described in Formula (I).

In some embodiments of the radiopharmaceutical conjugate, the peptide of Formula (I) has a structure of Formula (I-2):

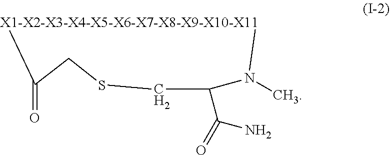

In some embodiments, the radiopharmaceutical conjugate comprises a peptide having an amino acid sequence that is at least 90% identical to a sequence selected from SEQ ID NOs: 1-72 or a sequence with up to 1, 2, 3, 4, or 5 substitutions by a conserved variant compared to any one of the sequences selected from SEQ ID NOs: 1-72. In some embodiments, the radiopharmaceutical conjugate comprises a peptide having an amino acid sequence with up to 1, 2, 3, 4, or 5 substitutions by a conserved variant compared to any one of the sequences selected from SEQ ID NOs: 1-72. In some embodiments, the radiopharmaceutical conjugate comprises a peptide having an amino acid sequence that is at least 90% identical to a sequence selected from SEQ ID NOs: 1-72. In some embodiments, the peptide consists of an amino acid sequence selected from SEQ ID NOs: 1-72.

In some embodiments, the radiopharmaceutical conjugate comprises a metal chelator configured to bind with a radionuclide, wherein the metal chelator is bound to the peptide of Formula (I) or SEQ ID NO: 1. In some embodiments, the radiopharmaceutical conjugate further comprises a linker that connects a peptide with a metal chelator. In some embodiments, the linker is attached to the peptide via a non-terminal amino acid residue of the peptide. In some embodiments, the linker is attached to the peptide via the N terminus of the peptide. In some embodiments, the linker is attached to the peptide via the C terminus of the peptide. In some embodiments, the linker is attached to the 1^{st} amino acid residue (or X1), the 2^{nd} amino acid residue (or X2), the 3^{rd} amino acid residue (or X3), the 4^{th} amino acid residue (or X4), the 8^{th} amino acid residue (or X8), or the 12^{th} amino acid residue (or X12). In some embodiments, the linker comprises 3 to 30 intervening non-hydrogen, organic atoms between the metal chelator and the peptide. In some embodiments, the linker comprises one or more amino acid residues. In some embodiments, the linker is a bond. In some embodiments, the metal chelator comprises DOTA, DOTA-GA, pBn-DOTA, pBn-SCN-DOTA, —NH2-DOTA. NH2-DOTA-GA, p-NCS-Bn-DOTA-GA, p-NH2-Bn-oxo-DO3A, p-SCN-Bn-oxo-DO3A, NOTA, NODA-GA, —NH2-NODA-GA, p-NCS-Bn-NODA-GA, p-NH2-Bn-NOTA, p-SCN-Bn-NOTA, NCS-MP-NODA, —NH2-MPAA-NODA, PCTA, p-NH2-Bn-PCTA, p-SCN-Bn-PCTA, p-SCN-Bn-HEHA, H2-MACROPA-NCS, H1-MACROPA. H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA. In some embodiments, the metal chelator has a structure of

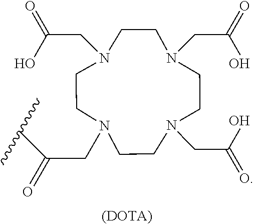 [SMILES: *C(=O)CN1CCN(CC(=O)O)CCN(CC(=O)O)CCN(CC(=O)O)CC1 ; "(DOTA)"]

In some embodiments, the radiopharmaceutical conjugate further comprises a radionuclide bound to the metal chelator. In some embodiments, the radionuclide is an alpha particle-emitting radionuclide. In some embodiments, the alpha particle-emitting radionuclide is Ac-225, Bi-213, Bi-209, Tb-149, Ra-223, Th-227, Fr-223, Gd-148. Th-229, Pb-212, or Po-213. In some embodiments, the alpha particle-emitting radionuclide is Ac-225. In some embodiments, the radionuclide is a beta particle-emitting radionuclide. In some embodiments, the beta particle-emitting radionuclide is Cu-67, Lu-177, Y-90, Rh-105, Yb-175, Tm-167, Pm-153, Sm-153, or In-111. In some embodiments, the beta particle-emitting radionuclide is lutetium-177. In some embodiments, the radionuclide is a positron-emitting radionuclide. In some embodiments, the positron-emitting radionuclide is Ga-68, Cu-62, Cu-64, Zr-89. Tb-152.

In some embodiments, the radiopharmaceutical conjugate comprises a covalently bound radionuclide, wherein the radionuclide is bound to the peptide of Formula (I) or SEQ ID NO: 1. In some embodiments, the radiopharmaceutical conjugate further comprises a linker that connects a peptide with the covalently bound radionuclide. In some embodiments, the radionuclide is covalently bound to an amino acid comprising an aromatic ring. In some embodiments, the radionuclide is attached to the 5^{th} amino acid or X5, the 8 amino acid or X8, the 9^{th} amino acid or X9, or the 11^{th} amino acid or X11. In some embodiments, the radionuclide is covalently bound to a residualizing agent attached to the peptide, wherein the residualizing agent has the structure of

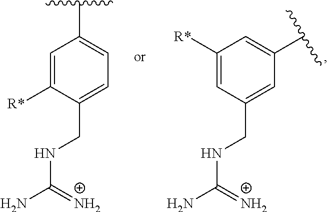

wherein R* is the covalently bound radionuclide. In some embodiments, the covalently bound radionuclide is ¹⁸F, ⁷⁴As, ⁷⁶Br, ¹²³I, ¹²⁴I, ¹²⁵I, ¹³¹I or ²¹¹At.

In one aspect, the present disclosure relates to a pharmaceutical composition comprising a radiopharmaceutical conjugate, or a pharmaceutically acceptable salt thereof, as described herein, a pharmaceutically acceptable excipient or carrier. In one aspect, the present disclosure relates to a pharmaceutical composition comprising a radiopharmaceutical or pharmaceutically acceptable salt thereof, as described herein, and a pharmaceutically acceptable excipient or carrier.

In one aspect, the present disclosure relates to a method of treating a disease or disorder characterized by overexpression of glypican 3 (GPC3) in a subject in need of treatment, the method comprising administering to the subject a therapeutically effective amount of a radiopharmaceutical or pharmaceutically acceptable salt thereof, as described herein, or a pharmaceutical composition as described herein. In one aspect, the present disclosure relates to a method of treating a disease or disorder characterized by overexpression of GPC3 in a subject in need of treatment, the method comprising: administering to the subject a first radiopharmaceutical or pharmaceutically acceptable salt thereof; and administering to the subject a therapeutically effective amount of a second radiopharmaceutical or pharmaceutically acceptable salt thereof, wherein: the first radiopharmaceutical or pharmaceutically acceptable salt thereof is described herein, and wherein the radionuclide is a diagnostic radionuclide; and the second radiopharmaceutical or pharmaceutically acceptable salt thereof is described herein, and wherein the radionuclide is a therapeutic radionuclide. In one aspect, the present disclosure relates to a method of diagnosing a disease or disorder characterized by overexpression of GPC3 in a subject in need thereof, comprising administering to the subject a radiopharmaceutical or pharmaceutically acceptable salt thereof, or pharmaceutical composition as described herein; and imaging the subject, thereby determining expression level of GPC3 in the subject, wherein the radionuclide is suitable for use as an imaging isotope. In one aspect, the present disclosure relates to a method of treating a disease or disorder characterized by overexpression of GPC3, in a subject in need of treatment, the method comprising administering to the subject the radiopharmaceutical conjugate or pharmaceutically acceptable salt thereof as described herein. In some embodiments, the disease or disorder is cancer. In some embodiments, the cancer is selected from hepatocellular carcinoma, squamous cell carcinoma of the lung, lung adenocarcinoma, germ cell tumors, hepatoblastoma, wilms tumor, malignant rhabdoid tumors, rhabdomyosarcoma, liposarcoma, thyroid cancers, pancreatic cancer, small bowel cancer, small cell neuroendocrine carcinoma (SCNC), hormonally treated, castration resistant prostatic adenocarcinoma, ovarian cancer, gastric cancer, esophageal carcinoma and malignant melanoma.

In one aspect, the present disclosure relates to a kit, tester, or composition for determining the expression level of GPC3 in a sample, wherein the kit, tester, or composition comprises the radiopharmaceutical or conjugate or pharmaceutically acceptable salt thereof as described herein. In some embodiments, the kit, tester, or composition is adapted for use in a method of diagnosing a disease or disorder characterized by an overexpression or a decreased expression of GPC3. In some embodiments, the sample is from a subject suspected of having a disease or disorder characterized by an overexpression or a decreased expression of GPC3.

In one aspect, the present disclosure relates to the use of the radiopharmaceutical, or conjugate, or pharmaceutically acceptable salt thereof as described herein in the manufacture of a medicament for diagnosing and/or treating a disease or disorder characterized by an overexpression or a decreased expression of GPC3.

In one aspect, the present disclosure relates to the use of the radiopharmaceutical, or conjugate, or pharmaceutically acceptable salt thereof as described herein for use in diagnosing and/or treating a disease or disorder characterized by an overexpression or a decreased expression of GPC3.

### INCORPORATION BY REFERENCE

All publications, patents, and patent applications mentioned in this specification are herein incorporated by reference for the specific purposes identified herein.

### BRIEF DESCRIPTION OF THE DRAWINGS

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 illustrates a flow chart of HepG2 cells, which have GPC3 on the cell surface, stained with biotinylated peptides followed by Streptavidin, R-Phycoerythrin Conjugate.

FIG. 2 illustrates a flow chart of Huh-7 cells, which have GPC3 on the cell surface, stained with biotinylated peptides followed by Streptavidin, R-Phycoerythrin Conjugate.

FIG. 3 illustrates a flow chart of SK-Hep1 cells, which was a negative control cell line, stained with biotinylated peptides followed by Streptavidin. R-Phycoerythrin Conjugate.

FIG. 4A illustrates exemplary metal chelators of the present disclosure, wherein [symbol] represents the attachment point of a metal chelator to the remaining conjugate. FIG. 4B illustrates the same metal chelators as FIG. 4A, except that a part of the linker or the peptide covalently connected to the metal chelator is shown in the dashed circle

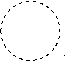

FIG. 5A illustrates exemplary metal chelators of the present disclosure, wherein [symbol] represents the attachment point of a metal chelator to the remaining conjugate. FIG. 5B illustrates the same metal chelators as FIG. 5A, except that a part of the linker or the peptide covalently connected to the metal chelator is shown in the dashed circle

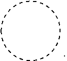

FIG. 6A illustrates exemplary metal chelators of the present disclosure, wherein [symbol] represents the attachment point of a metal chelator to the remaining conjugate. FIG. 6B illustrates the same metal chelators as FIG. 6A, except that a part of the linker or the peptide covalently connected to the metal chelator is shown in the dashed circle

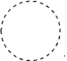

FIG. 7A illustrates exemplary metal chelators of the present disclosure, wherein [symbol] represents the attachment point of a metal chelator to the remaining conjugate. FIG. 7B illustrates the same metal chelators as FIG. 7A, except that a part of the linker or the peptide covalently connected to the metal chelator is shown in the dashed circle

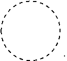

FIG. 8 illustrates the structures of representative metal chelators.

FIG. 9 illustrates the structures of representative metal chelators.

FIG. 10 illustrates the structures of representative metal chelators.

FIG. 11 illustrates the structures of representative metal chelators.

FIG. 12 illustrates the structures of representative metal chelators.

FIG. 13 illustrates the structures of representative metal chelators.

FIG. 14 illustrates the structures of representative metal chelators.

FIG. 15 illustrates the structures of representative metal chelators.

FIG. 16 illustrates the structures of representative metal chelators.

FIG. 17 illustrates the structures of representative metal chelators.

FIG. 18 illustrates the structures of representative metal chelators.

FIG. 19 illustrates the structures of representative metal chelators.

FIG. 20 illustrates the structures of representative metal chelators.

FIG. 21 illustrates the structures of representative metal chelators.

FIG. 22 illustrates the structures of representative metal chelators.

FIG. 23A illustrates a flow chart of SK-Hep1 cells, which do not have GPC3 expression on the cell surface, stained with a biotinylated GPC3-binding peptide of the invention (PLD-45), or a negative control, followed by Streptavidin, R-Phycoerythrin Conjugate.

FIG. 23B illustrates a flow chart of Huh-7 cells, which have GPC3 expression on the cell surface, stained with a biotinylated GPC3-binding peptide of the invention (PLD-45), or a negative control, followed by Streptavidin, R-Phycoerythrin Conjugate.

FIG. 23C illustrates a flow chart of HepG2 cells, which have GPC3 expression on the cell surface, stained with a biotinylated GPC3-binding peptide of the invention (PLD-45), or a negative control, followed by Streptavidin, R-Phycoerythrin Conjugate.

FIG. 24A illustrates a flow chart of SK-Hep1 cells, which do not have GPC3 expression on the cell surface, stained with a biotinylated GPC3-binding peptide of the invention (PLD-30), or a negative control, followed by Streptavidin, R-Phycoerythrin Conjugate.

FIG. 24B illustrates a flow chart of Huh-7 cells, which have GPC3 expression on the cell surface, stained with a biotinylated GPC3-binding peptide of the invention (PLD-30), or a negative control, followed by Streptavidin, R-Phycoerythrin Conjugate.

FIG. 24C illustrates a flow chart of HepG2 cells, which have GPC3 expression on the cell surface, stained with a biotinylated GPC3-binding peptide of the invention (PLD-30), or a negative control, followed by Streptavidin, R-Phycoerythrin Conjugate.

FIG. 25A illustrates a flow chart of SK-Hep1 cells, which do not have GPC3 expression on the cell surface, stained with a biotinylated GPC3-binding peptide of the invention (PLD-1), or a negative control, followed by Streptavidin, R-Phycoerythrin Conjugate.

FIG. 25B illustrates a flow chart of Huh-7 cells, which have GPC3 expression on the cell surface, stained with a biotinylated GPC3-binding peptide of the invention (PLD-1), or a negative control, followed by Streptavidin, R-Phycoerythrin Conjugate.

FIG. 25C illustrates a flow chart of HepG2 cells, which have GPC3 expression on the cell surface, stained with a biotiny lated GPC3-binding peptide of the invention (PLD-1), or a negative control, followed by Streptavidin, R-Phycoerythrin Conjugate.

FIG. 26A illustrates a flow chart of SK-Hep1 cells, which do not have GPC3 expression on the cell surface, stained with a biotinylated GPC3-binding peptide of the invention (Mod-3), or a negative control, followed by Streptavidin, R-Phycoerythrin Conjugate.

FIG. 26B illustrates a flow chart of Huh-7 cells, which have GPC3 expression on the cell surface, stained with a biotinylated GPC3-binding peptide of the invention (Mod-3), or a negative control, followed by Streptavidin, R-Phycoerythrin Conjugate.

FIG. 26C illustrates a flow chart of HepG2 cells, which have GPC3 expression on the cell surface, stained with a biotinylated GPC3-binding peptide of the invention (Mod-3), or a negative control, followed by Streptavidin, R-Phycoerythrin Conjugate.

FIG. 27A illustrates a flow chart of SK-Hep1 cells, which do not have GPC3 expression on the cell surface, stained with a biotinylated GPC3-binding peptide of the invention (Mod-5), or a negative control, followed by Streptavidin, R-Phycoerythrin Conjugate.

FIG. 27B illustrates a flow chart of Huh-7 cells, which have GPC3 expression on the cell surface, stained with a biotinylated GPC3-binding peptide of the invention (Mod-5), or a negative control, followed by Streptavidin, R-Phycoerythrin Conjugate.

FIG. 27C illustrates a flow chart of HepG2 cells, which have GPC3 expression on the cell surface, stained with a biotinylated GPC3-binding peptide of the invention (Mod-5), or a negative control, followed by Streptavidin, R-Phycoerythrin Conjugate.

FIG. 28A illustrates a flow chart of SK-Hep1 cells, which do not have GPC3 expression on the cell surface, stained with a biotinylated GPC3-binding peptide of the invention (Mod-6), or a negative control, followed by Streptavidin, R-Phycoerythrin Conjugate.

FIG. 28B illustrates a flow chart of Huh-7 cells, which have GPC3 expression on the cell surface, stained with a biotinylated GPC3-binding peptide of the invention (Mod-6), or a negative control, followed by Streptavidin, R-Phycoerythrin Conjugate.

FIG. 28C illustrates a flow chart of HepG2 cells, which have GPC3 expression on the cell surface, stained with a biotiny lated GPC3-binding peptide of the invention (Mod-6), or a negative control, followed by Streptavidin, R-Phycoerythrin Conjugate.

FIG. 29A illustrates a flow chart of SK-Hep1 cells, which do not have GPC3 expression on the cell surface, stained with a biotinylated GPC3-binding peptide of the invention (Mod-7), or a negative control, followed by Streptavidin, R-Phycoerythrin Conjugate.

FIG. 29B illustrates a flow chart of Huh-7 cells, which have GPC3 expression on the cell surface, stained with a biotinylated GPC3-binding peptide of the invention (Mod-7), or a negative control, followed by Streptavidin, R-Phycoerythrin Conjugate.

FIG. 29C illustrates a flow chart of HepG2 cells, which have GPC3 expression on the cell surface, stained with a biotinylated GPC3-binding peptide of the invention (Mod-7), or a negative control, followed by Streptavidin, R-Phycoerythrin Conjugate.

### DETAILED DESCRIPTION

It should be understood that both the general descriptions and the detailed description below are merely illustrative and descriptive and do not limit the present technology of the present application. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The headings used in the present specification are for structural purposes only and must not be construed as limiting the subject matter described.

In the present specification, the use of the singular form includes the plural form unless otherwise specified. In the present specification, the use of “or (or)” means “and/or (and/or)” unless otherwise stated. Furthermore, terms such as “element” or “component” encompass both an element and a component including one unit and an element and a component including two or more subunits unless when otherwise specified.

The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case. e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

### I. Definitions

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value.

As used herein, the term “conjugate” is used interchangeably with “radiopharmaceutical conjugate”. A conjugate described herein may comprise a radionuclide. In some embodiments, a conjugate described herein does not comprise a radionuclide.

As used herein, the term “radiopharmaceutical” refers to a conjugate comprising a radionuclide. In some embodiments, the radionuclide is a covalently bound to the conjugate. In some embodiments, the radionuclide is bound to the conjugate through a metal chelator.

The term “comprising” (and related terms such as “comprise” or “comprises” or “having” or “including”) are to be construed in an open, inclusive sense, that is, as “including, but not limited to.” The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Amino” refers to the —NH₂ radical.

“Cyano” refers to the —CN radical.

“Nitro” refers to the —NO₂ radical.

“Oxo” refers to the ═O radical.

“Imino” refers to the ═N—H radical.

“Oximo” refers to the ═N—OH radical.

“Hydrazino” refers to the ═N—NH₂ radical.

“Hydroxy” or “hydroxyl” refers to the —OH radical.

“Hydroxyamino” refers to the —NH—OH radical.

“Acyl” refers to a substituted or unsubstituted alkylcarbonyl, substituted or unsubstituted alkenylcarbonyl, substituted or unsubstituted alkynylcarbonyl, substituted or unsubstituted cycloalkylcarbonyl, substituted or unsubstituted heterocycloalkylcarbonyl, substituted or unsubstituted arylcarbonyl, substituted or unsubstituted heteroarylcarbonyl, amide, or ester, wherein the carbonyl atom of the carbonyl group is the point of attachment. Unless stated otherwise specifically in the specification, an alkylcarbonyl group, alkenylcarbonyl group, alkynylcarbonyl group, cycloalkylcarbonyl group, amide group, or ester group is optionally substituted, for example, with oxo, halogen, amino, nitrile, nitro, hydroxyl, haloalkyl, alkoxy, aryl, cycloalkyl, heterocycloalkyl, heteroaryl, and the like.

“Alkyl” refers to an optionally substituted straight-chain, or optionally substituted branched-chain saturated hydrocarbon monoradical. An alkyl group can have from one to about twenty carbon atoms, from one to about ten carbon atoms, or from one to six carbon atoms. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁₋₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —ONMe, —NH₂, —NO₂, or —C≡CH. In some embodiments, the alkyl is optionally substituted with oxo, halogen, —CN, —CF₃, —OH, or —ONMe. In some embodiments, the alkyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylene is optionally substituted with oxo, halogen, —CN, —CF₃, —OH, —OMe, —NH₂, or —NO₂. In some embodiments, an alkylene is optionally substituted with oxo, halogen, —CN, —CF₃, —OH, or —OMe. In some embodiments, the alkylene is optionally substituted with halogen. In some embodiments, the alkylene is —CH₂—, —CH₂CH₂—, —CH₂CH₂CH₂—, or —CH₂CH(CH₃)CH₂—. In some embodiments, the alkylene is —CH₂—. In some embodiments, the alkylene is —CH₂CH₂—. In some embodiments, the alkylene is —CH₂CH₂CH₂—.

“Alkenyl” refers to an optionally substituted straight-chain, or optionally substituted branched-chain hydrocarbon monoradical having one or more carbon-carbon double-bonds. In some embodiments, an alkenyl group has from two to about ten carbon atoms, or two to about six carbon atoms. The group may be in either the cis or trans configuration about the double bond(s), and should be understood to include both isomers. Examples include, but are not limited to, ethenyl (—CH═CH₂), 1-propenyl (—CH₂CH═CH₂), isopropenyl [—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₁-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

The term “alkenylene” or “alkenylene chain” refers to an optionally substituted straight or branched divalent hydrocarbon chain in which at least one carbon-carbon double bond is present linking the rest of the molecule to a radical group. In some embodiments, the alkenylene is —CH═CH—, —CH₂CH═CH—, or —CH═CHCH₂—. In some embodiments, the alkenylene is —CH═CH—. In some embodiments, the alkenylene is —CH₂CH═CH₂—. In some embodiments, the alkenylene is CH═CHCH₂—.

“Alkynyl” refers to an optionally substituted straight-chain or optionally substituted branched-chain hydrocarbon monoradical having one or more carbon-carbon triple-bonds. In some embodiments, an alkynyl group has from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₁-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen. The term “alkynylene” refers to an optionally substituted straight-chain or optionally substituted branched-chain divalent hydrocarbon having one or more carbon-carbon triple-bonds.

“Alkylamino” refers to a radical of the formula —N(Rₐ)₂ where Rₐ is an alkyl radical as defined, or two Rₐ taken together with the nitrogen atom, can form a substituted or unsubstituted C₂-C₇ heterocyloalkyl ring. Unless stated otherwise specifically in the specification, an alkylamino group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkylamino is optionally substituted with oxo, halogen, —CN, —CF₃, —OH, —OMe, —NH₂, or —NO₂. In some embodiments, an alkylamino is optionally substituted with oxo, halogen, —CN, —CF₃, —OH, or —OMe. In some embodiments, the alkylamino is optionally substituted with halogen.

“Alkoxy” refers to a radical of the formula —OR where R, is an alkyl radical as defined. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems. Unless stated otherwise specifically in the specification, an aryl may be optionally substituted, for example, with halogen, amino, alkylamino, aminoalkyl, nitrile, nitro, hydroxyl, alkyl, alkenyl, alkynyl, haloalkyl, heteroalkyl, alkoxy, aryl, cycloalkyl, heterocycloalkyl, heteroaryl, —S(O)₂NH—C₁-C₆alkyl, and the like. In some embodiments, an aryl is optionally substituted with halogen, methyl, ethyl, —CN, —CF₃, —OH, —OMe, —NH₂, —NO₂, —S(O)₂NH₂, —S(O)₂NHCH₃, —S(O)₂NHCH₂CH₃, —S(O)₂NHCH(CH₃)₂, —S(O)₂N(CH₃)₂, or —S(O)₂NHC(CH₃)₃. In some embodiments, an aryl is optionally substituted with halogen, methyl, ethyl, —CN, —CF₃, —OH, or —OMe. In some embodiments, the aryl is optionally substituted with halogen. In some embodiments, the aryl is substituted with alkyl, alkenyl, alkynyl, haloalkyl, or heteroalkyl, wherein each alkyl, alkenyl, alkynyl, haloalkyl, heteroalkyl is independently unsubstituted, or substituted with halogen, methyl, ethyl, —CN, —CF₃, —OH, —OMe, —NH₂, or —NO₂

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted. Representative cycloalkyls include, but are not limited to, cycloalkyls having from three to fifteen carbon atoms (e.g., C₃-C₁₅ fully saturated cycloalkyl or C₃-C₁₅ cycloalkenyl), from three to ten carbon atoms (e.g., C₃-C₁₀ fully saturated cycloalkyl or C₃-C₁₀ cycloalkenyl), from three to eight carbon atoms (e.g., C₃-C₈ fully saturated cycloalkyl or C₃-C₈ cycloalkenyl), from three to six carbon atoms (e.g., C₃-C₆ fully saturated cycloalkyl or C₃-C₆ cycloalkenyl), from three to five carbon atoms (e.g., C₃-C₅ fully saturated cycloalkyl or C₃-C₅ cycloalkenyl), or three to four carbon atoms (e.g., C₃-C₄ fully saturated cycloalkyl or C₃-C₄ cycloalkenyl). In some embodiments, the cycloalkyl is a 3-to 6-membered cycloalkyl. In some embodiments, the cycloalkyl is a 5-to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro, or iodo. In some embodiments, halogen is fluoro or chloro. In some embodiments, halogen is fluoro.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl can include, for example, iodoalkyl, bromoalkyl, chloroalkyl, and fluoroalkyl. For example, “fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆heteroalkyl wherein the heteroalkyl is comprised of 1 to 6 carbon atoms and one or more atoms other than carbon, e.g., oxygen, nitrogen (e.g, —NH—, —N(alkyl)-), sulfur, or combinations thereof wherein the heteroalkyl is attached to the rest of the molecule at a carbon atom of the heteroalkyl. Examples of such heteroalkyl are, for example, —CH₂—O—CH₂—, —CH₂—N(alkyl)-) CH₂—, —CH₂—N(aryl)-CH₂—, —OCH₂CH₂O—, —OCH₂CH₂OCH₂CH₂—, or —OCH₂CH₂OCH₂CH₂OCH₂OCH₂O—. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

As used herein, a “heteroalkylene” refers to divalent heteroalkyl group. Examples of such heteroalkylene are, for example, —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heterocycloalkyl” refers to a cycloalkyl group that includes at least one hetero ring atom, e.g., a heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quaternized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetralydrofuryl, trithianyl, tetrahydropyranyl, thiomorpholinyl, thiamorpholinyl, I-oxo-thiomorpholinyl, 1,1-dioxo-thiomorpholinyl. Representative heterocycloalkyls include, but are not limited to, heterocycloalkyls having from two to fifteen carbon atoms (e.g., C₂-C₁₅ fully saturated heterocycloalkyl or C₂-C₁₅ heterocycloalkenyl), from two to ten carbon atoms (e.g., C₂-C₁₀ fully saturated heterocycloalkyl or C₂-C₁₀ heterocycloalkenyl), from two to eight carbon atoms (e.g., C₂-C₈ fully saturated heterocycloalkyl or C₂-C₈ heterocycloalkenyl), from two to seven carbon atoms (e.g., C₂-C₇ fully saturated heterocycloalkyl or C₂-C₇ heterocycloalkenyl), from two to six carbon atoms (e.g., C₂-C₆ fully saturated heterocycloalkyl or C₂-C₇ heterocycloalkenyl), from two to five carbon atoms (e.g., C₂-C₅ fully saturated heterocycloalkyl or C₂-C₅ heterocycloalkenyl), or two to four carbon atoms (e.g., C₂-C₄ fully saturated heterocycloalkyl or C₂-C₄ heterocycloalkenyl). The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a ring system radical comprising carbon atom(s) and one or more ring heteroatoms selected from the group consisting of nitrogen, oxygen, phosphorous, and sulfur, and at least one aromatic ring. In some embodiments, heteroaryl is monocyclic, bicyclic or polycyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7,8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

The term “moiety” refers to a specific segment or functional group of a molecule. Chemical moieties are often recognized chemical entities embedded in or appended to a molecule.

The terms “treat,” “prevent,” “ameliorate,” and “inhibit,” as well as words stemming therefrom, as used herein, do not necessarily imply 100% or complete treatment, prevention, amelioration, or inhibition. Rather, there are varying degrees of treatment, prevention, amelioration, and inhibition of which one of ordinary skill in the art recognizes as having a potential benefit or therapeutic effect. In this respect, the disclosed methods can provide any amount of any level of treatment, prevention, amelioration, or inhibition of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%. Furthermore, the treatment, prevention, amelioration, or inhibition provided by the methods disclosed herein can include treatment, prevention, amelioration, or inhibition of one or more conditions or symptoms of the disorder, e.g., cancer or an inflammatory disease.

In certain embodiments, “treating” includes the concepts of “alleviating,” which refers to lessening the frequency of occurrence or recurrence, or the severity, of any symptoms or other ill effects related to a disorder and/or the associated side effects. In certain embodiments, the term “treating” also encompasses the concept of “managing” which refers to reducing the severity of a particular disease or disorder in a patient or delaying its recurrence, e.g., lengthening the period of remission in a patient who had suffered from the disease.

In certain embodiments, the term “prevent” or “preventing” as related to a disease or disorder can refer to a compound that in a statistical sample, reduces the occurrences of the disorder or condition in the treated sample relative to an untreated control sample, or delays the onset or reduces the severity of one or more symptoms of the disorder or condition relative to the untreated control sample.

The term “therapeutically effective amount” as used herein refers to an amount effective at the dosage and duration necessary to achieve the desired therapeutic result. A therapeutically effective amount of the composition may vary depending on factors such as the individual's condition, age, sex, and weight, and the ability of the protein to elicit the desired response of the individual. A therapeutically effective amount can also be an amount that exceeds any toxic or deleterious effect of the composition that would have a beneficial effect on the treatment.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.).

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances one or more substituents having a double bond (e.g., “oxo” or “═O”) as the point of attachment may be described, shown or listed herein within a substituent group, wherein the structure may only show a single bond as the point of attachment to the core structure. A person of ordinary skill in the art would understand that, while only a single bond is shown, a double bond is intended for those substituents.

For the purpose of the disclosure, one event of “substitution” of an amino acid or an amino sequence is not considered two separate events of one deletion plus one addition. Thus, for the avoidance of doubt, as an example, a sequence change of “up to two deletion, substitution and/or addition” includes one deletion and one substitution, one deletion and one addition (at a different position), one substitution and one addition, one deletion only, one substitution only, one addition only, two deletions, two substitutions, two additions, etc. The deletion, addition, or substitution position may be at one or both ends of the peptide, or in the middle of the peptide.

The term “optionally substituted” or “substituted” means that the referenced group is optionally substituted with one or more additional group(s). For example, “optionally substituted” or “substituted” can mean that the referenced group is optionally substituted with one or more substituents individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, an “optionally substituted” group is independently substituted with 1-6 substituents selected from halogen, —CN, oxo, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₆haloalkyl, C₁-C₆haloalkoxy, C₁-C₆hydroxyalkyl, C₁-C₆aminoalkyl, C₁-C₆heteroalkyl, aryl, heteroaryl, heterocycloalkyl and cycloalkyl. In some embodiments, an “optionally substituted” group is independently substituted with 1-6 substituents selected from halogen, —CN, oxo, —OH, —SF₅, —SH, —S(═O)C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, 5-6 membered heterocycloalkyl and C₃-C₆cycloalkyl. In some embodiments, an “optionally substituted” group is independently substituted with 1-6 substituents selected from halogen, oxo, —OH, —NH₂, —NHC₁-C₃alkyl, —N(C₁-C₃alkyl)₂, —C(═O)OH, —C(═O)NH₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, and C₃-C₆cycloalkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O). When indicating the number of substituents, the term “one or more” means from one substituent to the highest possible number of substitutions, i.e. replacement of one hydrogen up to replacement of all hydrogens by substituents. In some embodiments, an “optionally substituted” group is unsubstituted. In some embodiments, an “optionally substituted” group is independently substituted with 1-6 substituents. In some embodiments, an “optionally substituted” group is independently substituted with 1-3 substituents. In some embodiments, an “optionally substituted” group is independently substituted with 1-2 substituents.

The term “unsubstituted” means that the specified group bears no substituents.

Certain compounds described herein may exist in tautomeric forms, and all such tautomeric forms of the compounds being within the scope of the disclosure.

Unless otherwise stated, structures depicted herein are also meant to include all stereochemical forms of the structure: i.e., the R and S configurations for each asymmetric center. Therefore, single stereochemical isomers as well as enantiomeric and diastereomeric mixtures of the present compounds are within the scope of the disclosure.

The term “peptide” as used herein refers to a compound that includes two or more amino acids. A peptide described herein can comprise one or more unnatural amino acids. The term “peptide” also encompasses peptide mimetics. In the present disclosure, the term “amino acid” is used in its broadest meaning and it embraces not only natural amino acids but also derivatives thereof and artificial amino acids. For example, the term “amino acid” encompasses unnatural amino acids.

The term “peptoid” as used herein refers to an N-substituted glycine. A peptoid can be optionally substituted. A peptoid can optionally comprise additional substitutions at the alpha-carbon.

As used herein, the term “unnatural amino acid” refers to an amino acid other than the 20 canonical amino acids. The 20 canonical amino acids refer to alanine (ala or A), arginine (arg or R), asparagine (asn or N), aspartic acid (asp or D), cysteine (cys or C), glutamine (gln or Q), glutamic acid (glu or E), glycine (gly or G), histidine (his or H), isoleucine (ile or 1), leucine (leu or L), lysine (lys or K), methionine (met or M), phenylalanine (phe or F), proline (pro or P), serine (ser or S), threonine (thr or T), tryptophan (trp or W), tyrosine (tyr or Y), and valine (val or V).

The term “protein” as used herein refers to a polypeptide (i.e., a string of at least 3 amino acids linked to one another by peptide bonds). Proteins can include moieties other than amino acids (e.g., may be glycoproteins, proteoglycans, etc.) and/or can be otherwise processed or modified. A protein can be a complete polypeptide as produced by and/or active in a cell (with or without a signal sequence). In some embodiments, a protein is or comprises a characteristic portion such as a polypeptide as produced by and/or active in a cell. A protein can include more than one polypeptide chain. For example, polypeptide chains can be linked by one or more disulfide bonds or associated by other means.

The term “peptide mimetic” or “mimetic” refers to biologically active compounds that mimic the biological activity of a peptide or a protein but are no longer entirely peptidic in chemical nature, e.g., they can contain non-peptide bonds (that are, bonds other than amide bonds between amino acids). As used herein, the term peptide mimetic is used in a broader sense to include molecules that are no longer completely peptidic in nature, such as pseudo-peptides, semi-peptides and peptoids. Whether completely or partially non-peptide, peptide mimetics described herein can provide a spatial arrangement of reactive chemical moieties that closely resemble the three-dimensional arrangement of active groups in the subject amino acid sequence or subject molecule on which the peptide mimetic is based. As a result of this similar active-site geometry, the peptide mimetic can have effects on biological systems that are similar to the biological activity of the subject entity.

In some embodiments, the peptide mimetics are substantially similar in both three-dimensional shape and biological activity to the subject amino acid sequence or subject molecule on which the peptide mimetic is based. An example is described in the paper “Tritiated D-alal-Peptide T Binding”, Smith C. S. et al., Drug Development Res., 15, pp. 371-379 (1988). A second method is altering cyclic structure for stability, such as N to C interchain imides and lactams (Ede et al. in Smith and Rivier (Eds.) “Peptides: Chemistry and Biology”, Escom, Leiden (1991), pp. 268-270). An example of this is provided in conformationally restricted thymopentin-like compounds, such as those disclosed in U.S. Pat. No. 4,457,489. A third method is to substitute peptide bonds in the subject entity by pseudopeptide bonds that confer resistance to proteolysis.

The term “organic atoms” refers to atoms which would be found in organic compounds, such as carbon, hydrogen, nitrogen, oxygen, surfur, phosphorus, fluorine, chlorine, bromine, or iodine

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 12, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein, C₁-Cₓ(or C₁₋ₓ) includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Also, by way of example, C₀-C₂ alkylene includes a direct bond, —CH₂—, and —CH₂CH₂— linkages.

The term “cyclized” or “cyclization” as used herein means that two amino acids apart from each other by at least one amino acid bind directly or bind indirectly to each other in one peptide to form a cyclic structure in the molecule. In some cases, the two amino acids bind via a linker or the like.

The term “subject” or “patient” encompasses mammals. Examples of mammals include, but are not limited to, any member of the Mammalian class: humans, non-human primates such as chimpanzees, and other apes and monkey species: farm animals such as cattle, horses, sheep, goats, swine; domestic animals such as rabbits, dogs, and cats; laboratory animals including rodents, such as rats, mice and guinea pigs, and the like. In one aspect, the mammal is a companion animal such as a dog or a cat. In one aspect, the mammal is a human.

Percent sequence identity can be calculated using computer programs or direct sequence comparison. Preferred computer program methods to determine identity between two sequences include, but are not limited to, the GCG program package, FASTA, BLASTP, and TBLASTN (see, e.g., D. W. Mount, 2001, Bioinformatics: Sequence and Genome Analysis, Cold Spring Harbor Laboratory Press, Cold Spring Harbor, N.Y.). The BLASTP and TBLASTN programs are publicly available from NCBI and other sources. The Smith Waterman algorithm can also be used to determine percent identity. Exemplary parameters for amino acid sequence comparison include the following: 1) algorithm from Needleman and Wunsch (J. Mol. Biol., 48:443-453 (1970)); 2) BLOSSUM62 comparison matrix from Hentikoff and Hentikoff (Proc. Nat. Acad. Sci. USA., 89:10915-10919 (1992)) 3) gap penalty=12; and 4) gap length penalty=4. A program useful with these parameters can be publicly available as the “gap” program (Genetics Computer Group, Madison, Wis.). The aforementioned parameters are the default parameters for polypeptide comparisons (with no penalty for end gaps). Alternatively, polypeptide sequence identity can be calculated using the following equation: % identity−(the number of identical residues)/(alignment length in amino acid residues)*100. For this calculation, alignment length includes internal gaps but does not include terminal gaps.

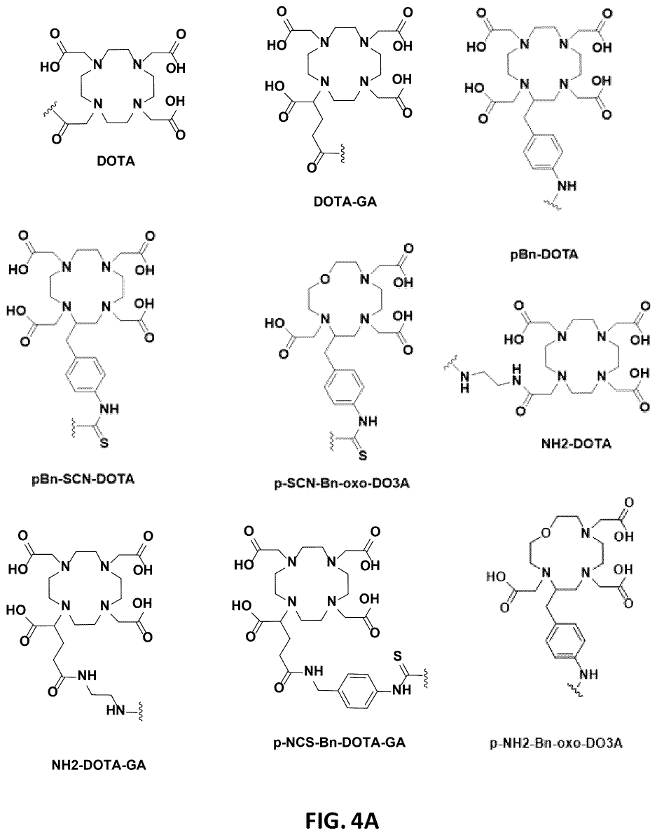

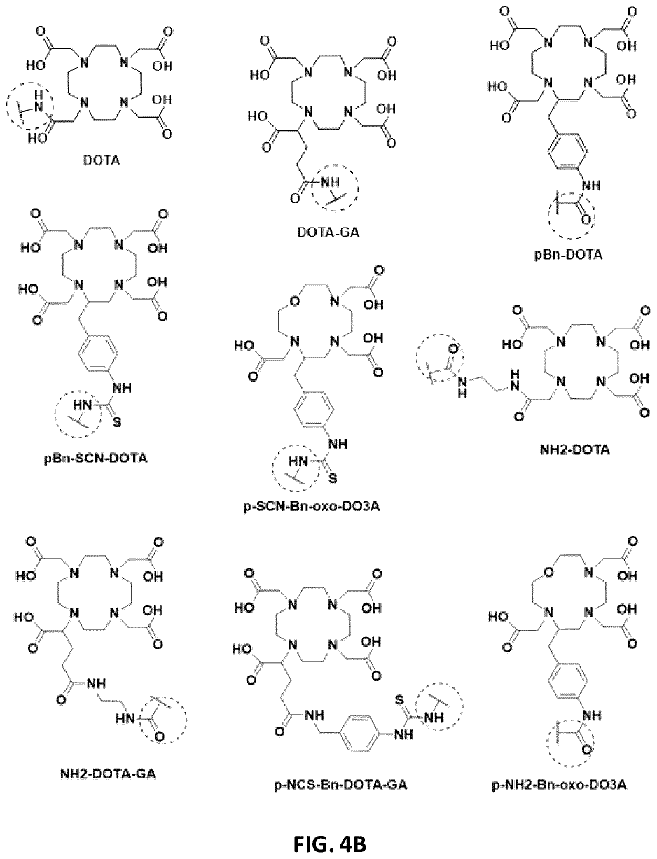

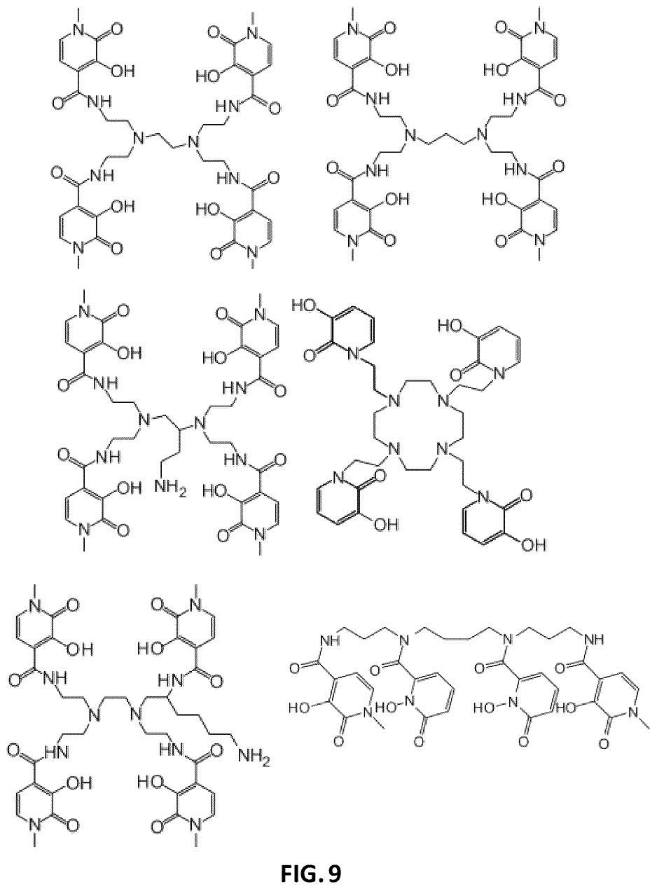

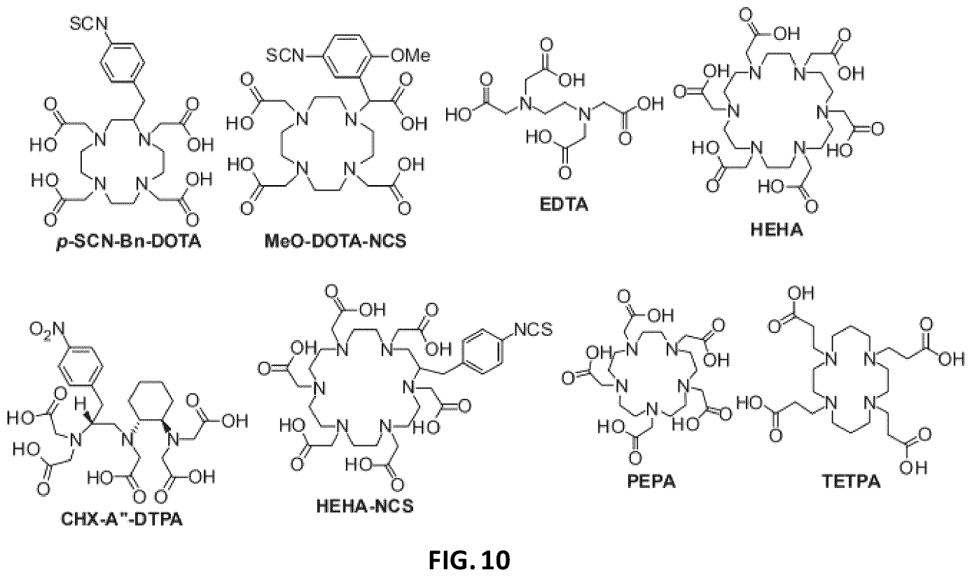

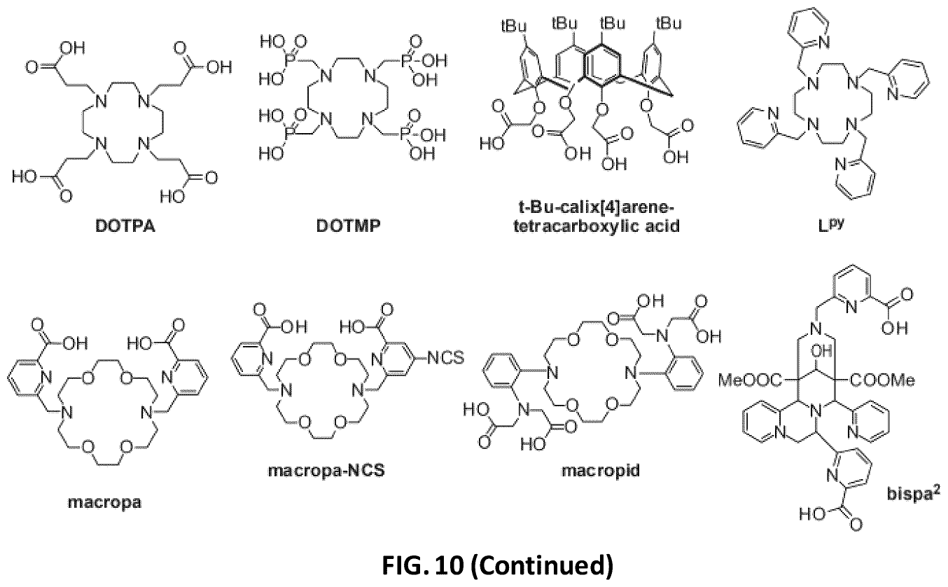

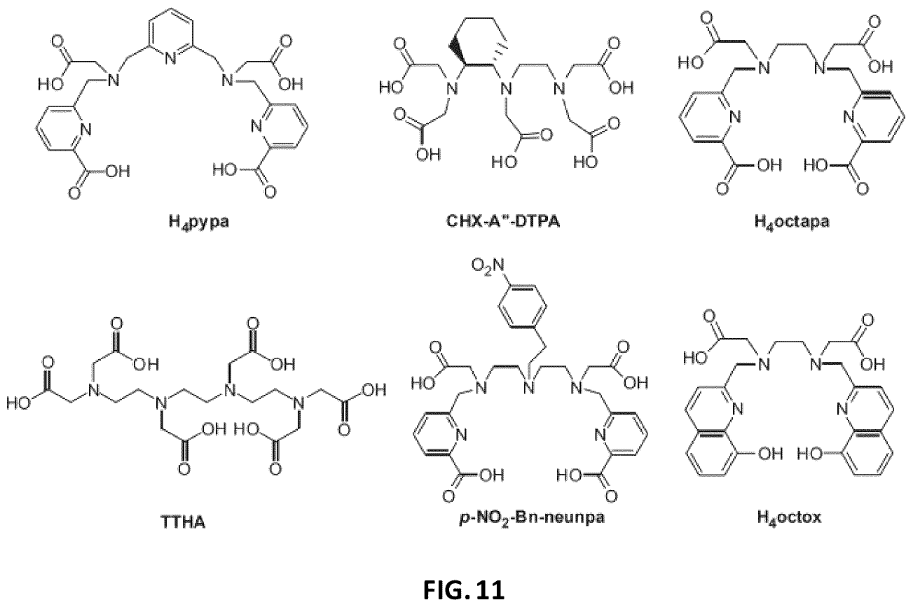

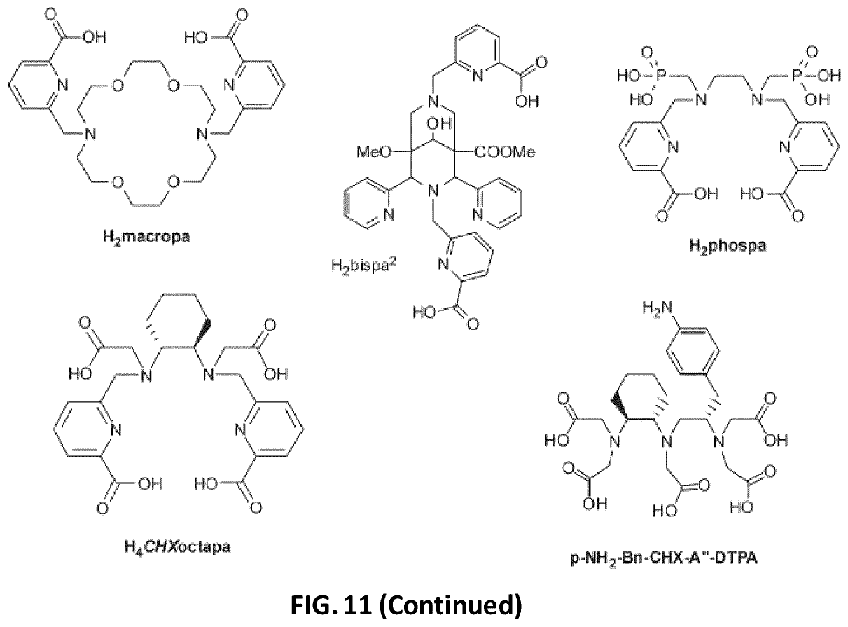

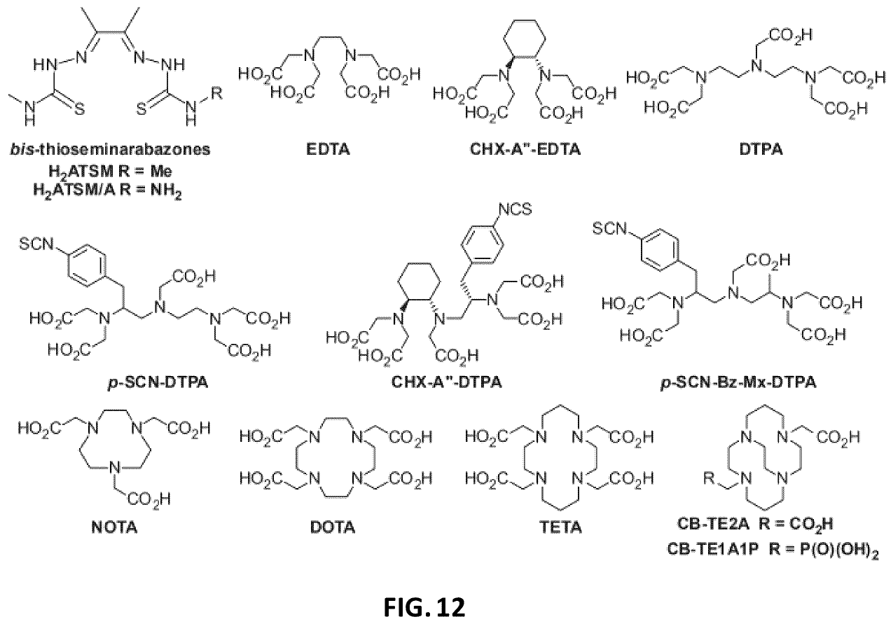

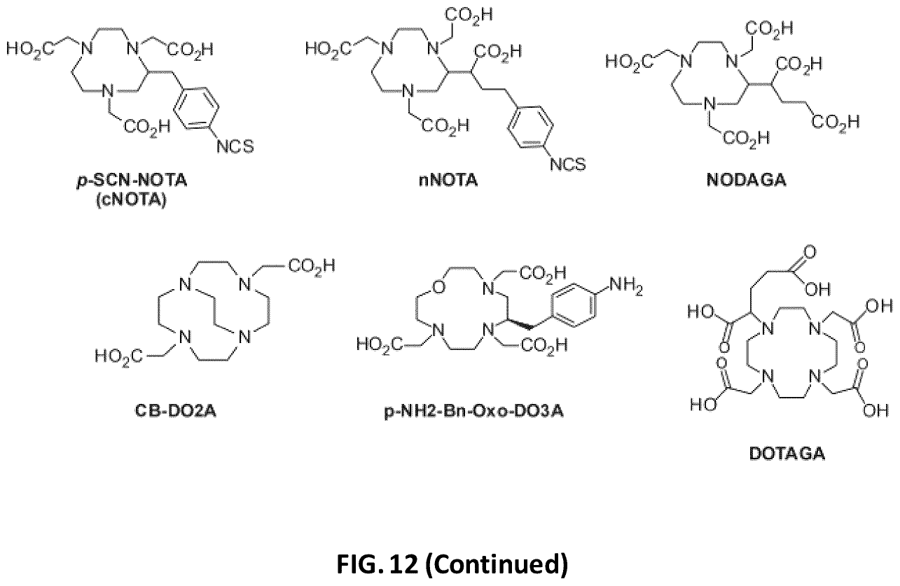

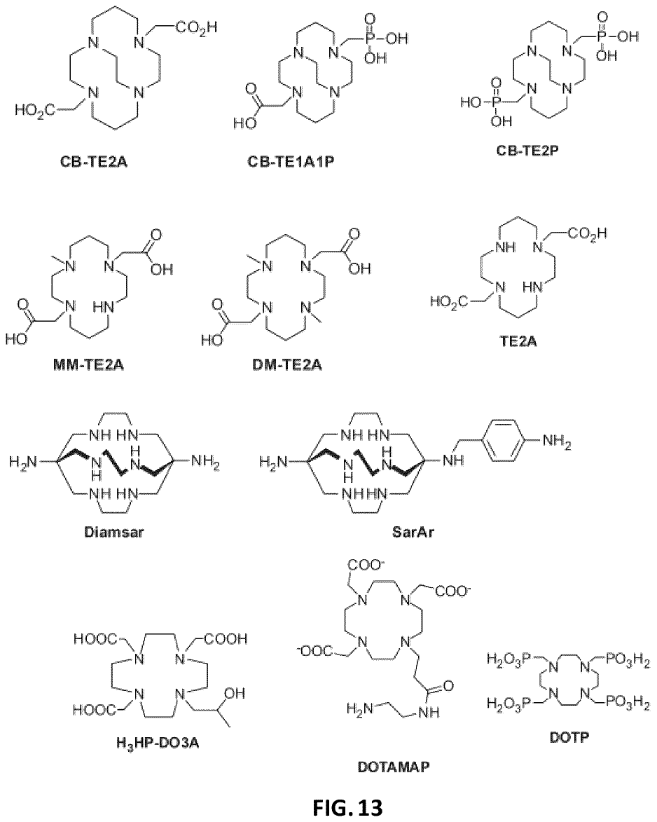

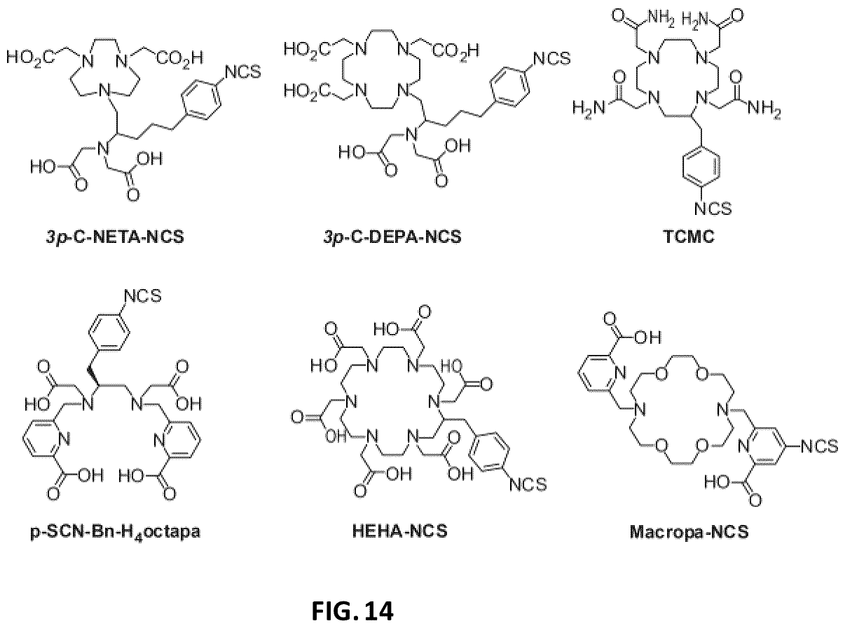

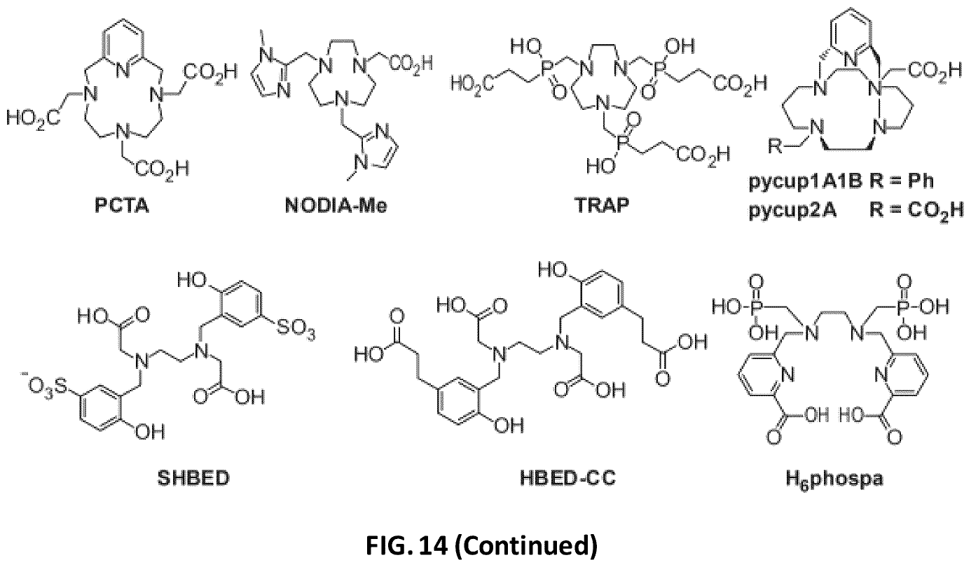

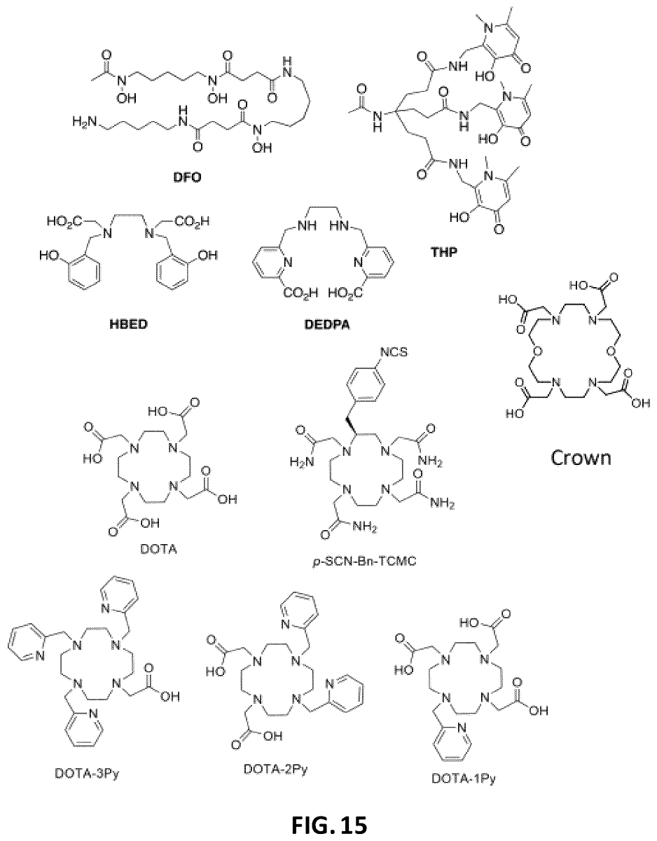

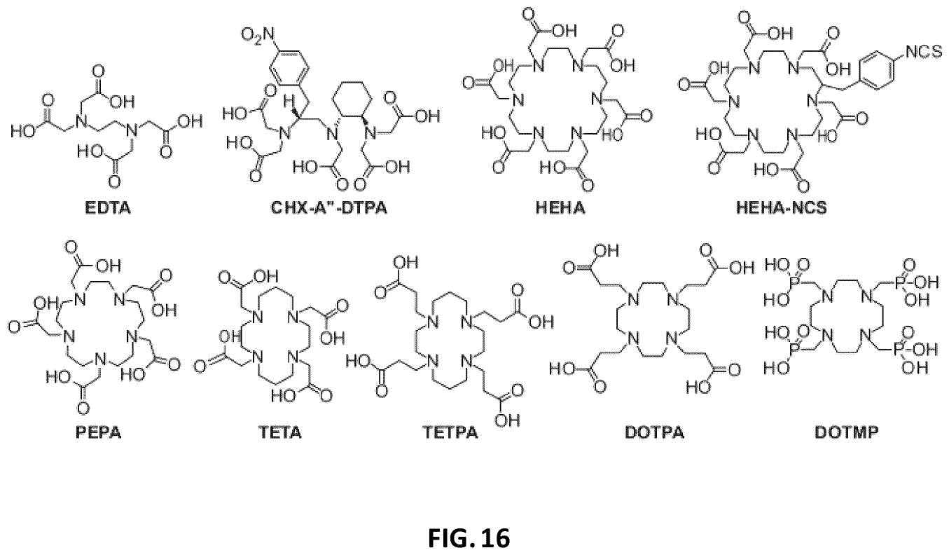

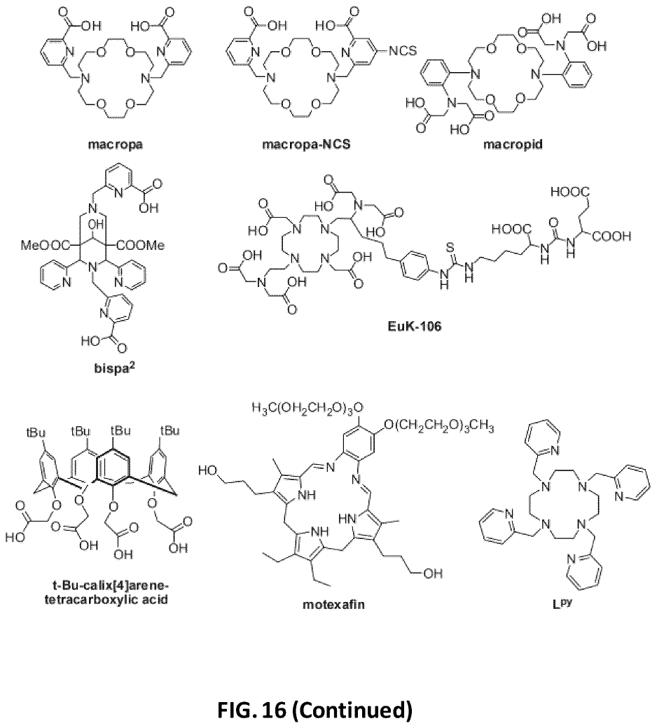

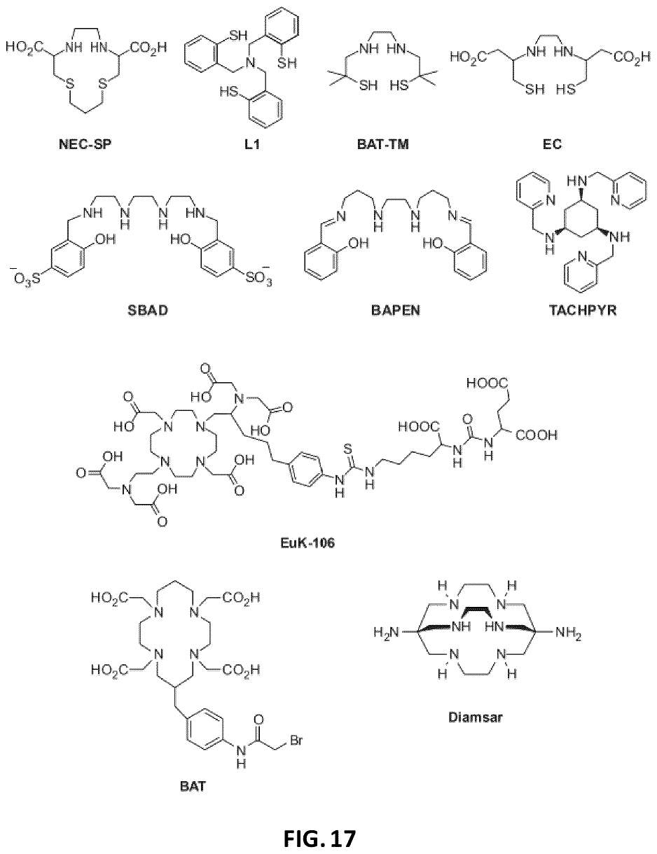

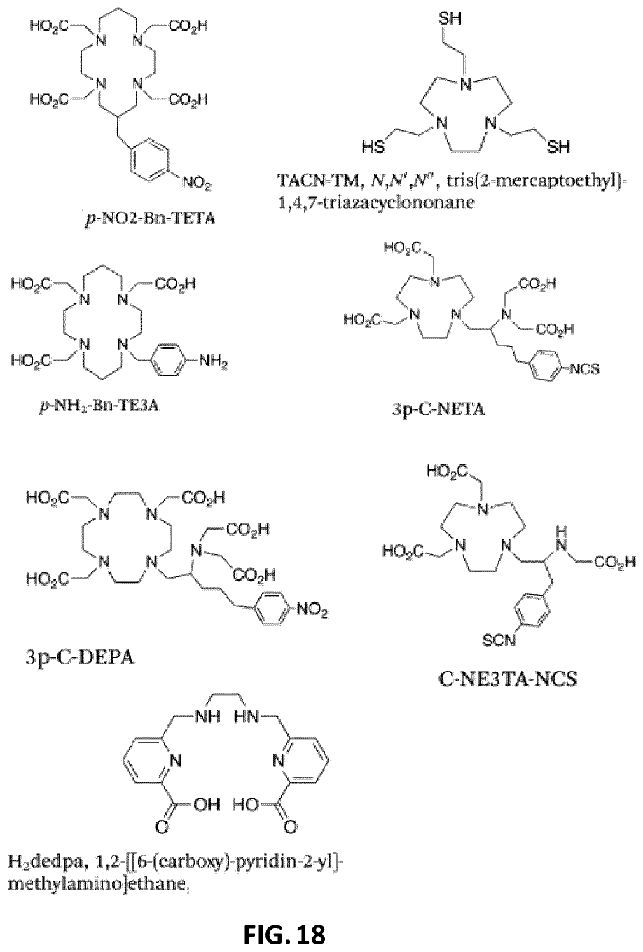

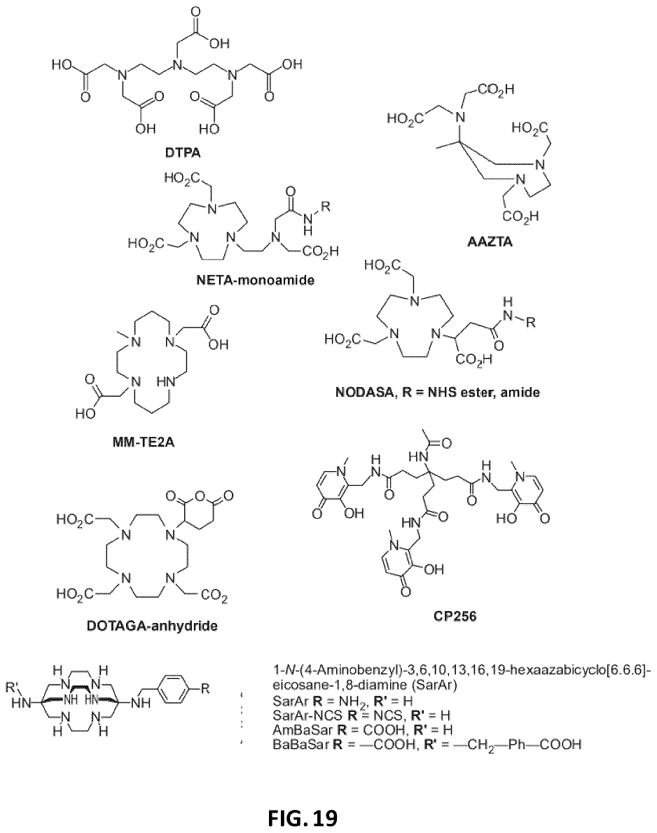

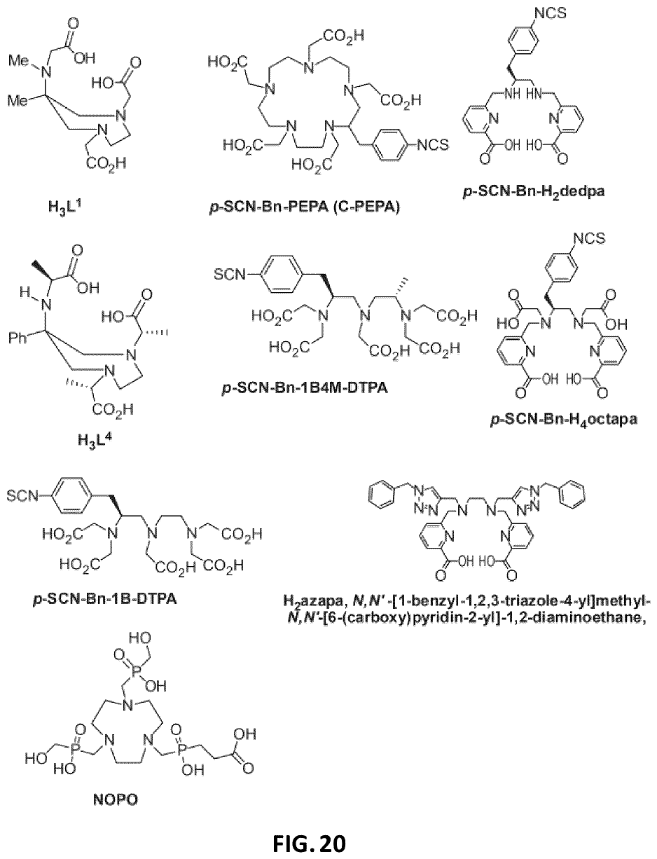

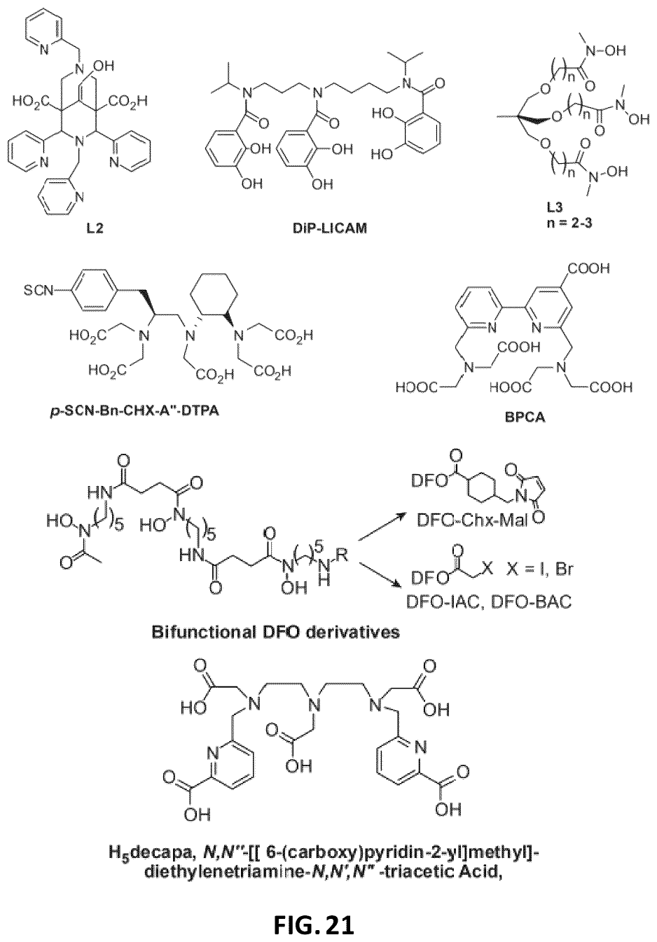

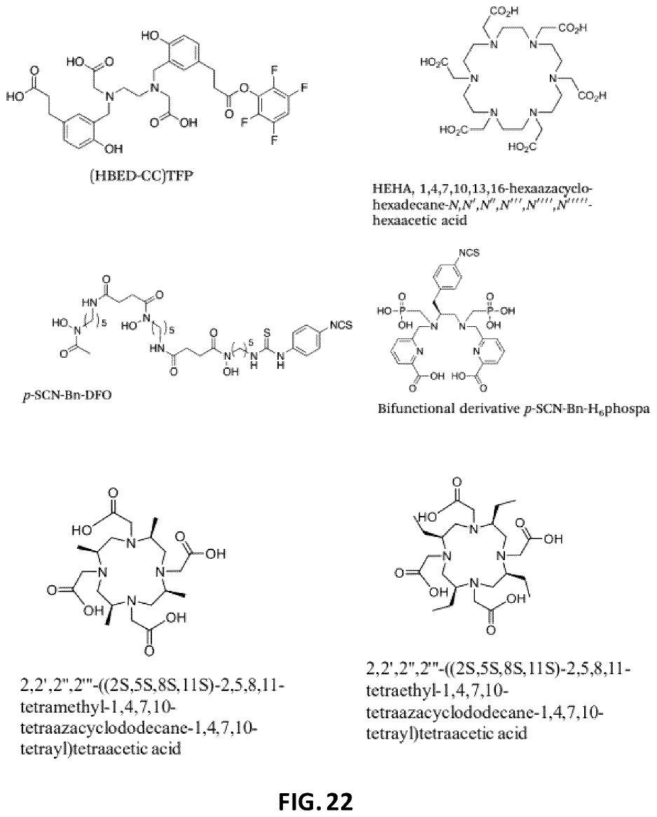

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable subcombination. For example, a conjugate of this disclosure can comprise any peptide ligand described herein (e.g., a peptide ligand of Formula (I), (I-1), (I-2), (I-3), (I-4), (I-5), or Table 1), any metal chelator described herein (e.g., a metal chelator selected from FIGS. 4A, 5A, 6A, 7A, 4B, 5B, 6B, 7B and 8-22), optionally a linker described herein (e.g., a linker of Formula (II-1), (II-1a), or (II-1b)), and optionally a radionuclide described herein (e.g., a radionuclide of Table 7). For another example, a peptide of Formula (I) (or any other Formulae such as (III-1) and (III-2)) can comprise X1 to X12 amino acids as described herein, and any combinations of the embodiments of amino acids are encompassed by this disclosure (even though, in some cases, they are described in the context of separate embodiments).

Unless special definitions are given, the terminology used in relation to analytical chemistry, synthetic organic chemistry, and medical chemistry and pharmaceutical chemistry described in the present specification, as well as their procedures and techniques, are well known and commonly used in the field of the present art. Standard techniques may be used for chemical synthesis and chemical analysis. Those defined from among such techniques and procedures can be found in, for example, “K. J. Jensen. P. T. Shelton, S. L. Pedersen, Peptide Synthesis and Applications, 2nd Edition, Springer, 2013” and the like, and these are incorporated into the present specification by reference for all purposes. All patents, applications, published applications, and other publications, and other data referred to throughout the entire disclosure, when permitted, are incorporated into the present specification by reference.

### IL. Radiopharmaceutical Conjugates

Provided herein are radiopharmaceutical conjugates that have avidity for Glypican 3 (GPC3), or a pharmaceutically acceptable salt thereof, and pharmaceutical compositions comprising the conjugates. The conjugates and compositions can be useful for treating cancer. The conjugates and compositions can also be useful in imaging and disease diagnosis.

In one aspect, described herein is a conjugate that comprises a peptide that has avidity for Glypican 3 (GPC3) and a metal chelator that is configured to bind with a radionuclide. In some embodiments, the GPC3 is a human GPC3. The peptide can be cyclic or acyclic, and it can be monocyclic, bicyclic or poly cyclic. In one aspect, described herein is a conjugate that comprises a cyclic peptide and a metal chelator that is configured to bind with a radionuclide. In some embodiments, the peptide (such as cyclic peptide) is configured to bind to a target. A conjugate described herein can further comprises a linker that covalently attaches the peptide to the metal chelator. In some embodiments, the conjugate comprises a radionuclide such as ²²⁵Ac bound to the metal chelator.

In another aspect, described herein is a conjugate that comprises a peptide that has avidity for Glypican 3 (GPC3) and a covalently bound radionuclide. In some embodiments, the GPC3 is a human GPC3. The peptide can be cyclic or acyclic, and it can be monocyclic, bicyclic or polycyclic. In one aspect, described herein is a conjugate that comprises a cyclic peptide and a metal chelator that is configured to bind with a radionuclide. In some embodiments, the peptide (such as cyclic peptide) is configured to bind to a target. A conjugate described herein can further comprises a linker that links the covalent radionuclide to the peptide. In some embodiments, the conjugate comprises a covalent radionuclide such as ¹³¹I bound.

In one aspect, described herein is a radiopharmaceutical conjugate comprising:
(a) a peptide that has avidity for Glypican 3 (GPC3), or a pharmaceutically acceptable salt thereof, wherein the peptide comprises an amino acid sequence including deletion, substitution, and/or addition of one or several (e.g., 1-6) amino acids in the amino acid sequence of SEQ ID NO: 1:

(SEQ ID NO: 1)
MeK-MeI-D-MeQ-F4COO-I-I-Y-MeNal27N-G-3Py6Ph-MeC,

wherein the peptide consists of 10 to 12 amino acid residues; and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide. In some embodiments, the radiopharmaceutical conjugate comprises a radionuclide bound to the metal chelator.

In some embodiments, the radiopharmaceutical conjugate comprises a covalently bound radionuclide.

In some embodiments, the peptide is a cyclic peptide.

In some embodiments, the radiopharmaceutical conjugate comprises a metal chelator configured to bind with a radionuclide, wherein the metal chelator is bound to the peptide. In some embodiments, the radiopharmaceutical conjugate comprises a metal chelator configured to bind with a radionuclide, wherein the metal chelator is covalently linked to the peptide. In some embodiments, a radionuclide is bound to the metal chelator. In some embodiments, the radiopharmaceutical conjugate comprises a covalently bound radionuclide. In some embodiments, the peptide comprises an amino acid sequence with deletion of 2 or fewer amino acids in the amino acid SEQ ID NO: 1. In some embodiments, 1 amino acid is deleted from SEQ ID NO: 1. In some embodiments, the 1 amino acid deleted form SEQ ID NO: 1 is the MeI at position 2. In some embodiments, the 1 amino acid deleted form SEQ ID NCO: 1 is the D at position 3. In some embodiments, 1, 2, 3, 4, or 5 amino acids are added to the peptide of SEQ ID NO: 1. In some embodiments. 1, 2, or 3 amino acids are added to the peptide of SEQ ID NO: 1. In some embodiments. 1 or 2 amino acids are added to the peptide of SEQ ID NO: 1. In some embodiments, 1 amino acid is added to the peptide of SEQ ID NO: 1. In some embodiments, 2 amino acids are added to the peptide of SEQ ID NO: 1.

In one aspect, described herein is a radiopharmaceutical conjugate comprising:
(a) a peptide that has avidity for Glypican 3 (GPC3), or a pharmaceutically acceptable salt thereof, wherein the peptide comprises an amino acid sequence of Formula (I),

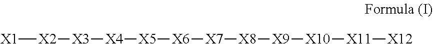

wherein,
   X1 is any amino acid;
   X2 is any amino acid;
   X3 is any amino acid:
   X4 is any amino acid;
   X5 is an amino acid comprising an aromatic ring (e.g., W, F, Y, or a variant thereof), cycloalkyl, or heterocycloalkyl group, or X5 is a peptoid (e.g., Cha4cH, Cha4tH, A1mor, Atp, Cha4cOMe);
   X6 is a hydrophobic amino acid, a hydrophilic amino acid, or a polar amino acid wherein the polar amino acid has a substituted side chain;
   X7 is a hydrophobic amino acid comprising a C₁-C₈ alkyl, cycloalkyl, or heterocycloalkyl, wherein the alkyl, cycloalkyl, and heterocycloalkyl are each independently, optionally substituted (e.g., X7 is 1, Eva, alI, TMe, SMe, Gcpr, Gcpe, Gthp, dMeS, TdMe, or Cbg);
   X8 is an A, 1, L, V, Y. or F, or a variant thereof;
   X9 is an N-alkylated amino acid comprising an aromatic ring;
   X10 is G, A, or a D-amino acid (e.g., da, ds, de, or dp);
   X11 is an amino acid comprising an aromatic ring (e.g., F, Y, or a variant thereof); and,
   X12 is N-alkylated cysteine (e.g., MeC); and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide. In some embodiments, the radiopharmaceutical conjugate comprises a radionuclide bound to the metal chelator. In some embodiments, the radiopharmaceutical conjugate comprises a covalently bound radionuclide.

In one aspect, described herein is a radiopharmaceutical conjugate comprising:
(a) a peptide that has avidity for Glypican 3 (GPC3), or a pharmaceutically acceptable salt thereof, wherein the peptide comprises an amino acid sequence of Formula (I),

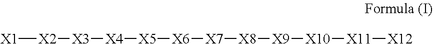

wherein,
   X1 is 1, R, Cit, F4G, 4Py, 3Py, KCOpipzaa, V, Eva, Q, E, MeI Ahp, F4COO, KCOpip4COO, MeQdMe, MeA, MeSMe, MeG, MeV, MeHseMe, Aib, MeT, alI, TMe, MeKCOpipzaa, MeQ, Hpr, MeTMe, MeDapCOpipzaa, MeK, MeKAc, MeK(de), MeK(H), MeK(df), or MeK(datb);
   X2 is I, K, Cit, F4G, 4Py, 3Py, KCOpipzetOH, V, KCOpipzaa, Eva, Q, E, S, Ahp, F4COO, KCOpip4COO, MeQdMe, MeA, MeSMe, MeG, MeI, MeV, MeL, HseMe, MeY, Me3Py, MeHseMe, MeKAc, alI, TMe MeKCOpipzaa, MeQ, Hpr, MeTMe, or MeDapCOpipzaaa;
   X3 is D, liar, KCOpipzetOH, Cit, KCOmeglumine, KCOpipzaa, A4paa, Q, A, E, MeD, S, N, Hgl, F4COO, KCOpip4COO, KAc, Hgn, MeY, or DapCOpipzaa;
   X4 is D, Har, KCOpipzetOH, KCOmeglumine, KCOpipzaa, A4paa, Q, A, E, MeD, S, N, Hgl, F4COO, KCOpip4COO, dd, MeQ, MeQdMe, MeA, MeSMe, MeG, EtG, MeeG, CmG, CmpG, CrmG, CeG, CrpG, MeK, MeKAc, MeHgl, Hgn, MeDapCOpipzaa, MeKCOpipzaa, Medd, Cit, MeCit, MeN, MeS, MeE, MeY, W5N, or MeA4paa;
   X5 is Y, F3G, 3Py6COO,4Py2NH2, 3Py5COO, F3COO, 3Py6NHAc, F, F4C, F40Me, F4COO, Nal2, F3aao, F4aa, F4aao, 3Py6NHaa, 5Pdo, F3CON, F4F, F40Et, F4Me, F4CON, F4CONPEG4Me, F30Me, F3CON, Yae, YaeCOpipzaa, F4aaopipzaa, 4Pdo, 3Py6CON, Atp, Cha4cH, Cha4tH, Cha4cOMe, A1mor, or F4amCOpipzaa;
   X6 is 1, V, Eva, Chg, Tbg, A, L, Ahp, F4COO, Gcpr, Gcpe, alI, Cle, S3REt, TMe, Acpr, Cba, Gthp, NleCOO, NieOH, P, Atb, Nva, Nie, N, DapAc, Abu, Nmn, Ndm, Ncit, Cit, SMe, HseMe, HseEt, HseiPr, dMeS, TdMe, Cbg, NyaOMe, SiPr, Spr, NlcOMe, Sbu, Scbm, Scpe, AhpOMe, HseBu, Spent or Hsecpe;
   X7 is I, Eva, alI, TMe, SMe, Gcpr, Gcpe, Gthp, dMeS, TdMe, or Cbg;
   X8 is A, 1, L, V, Y, F40Me, F4COO, F40Et, F4u, F4Me, F4CONdMe, F4CON, F4 ms, F4CONPEG4Me, F34dOMe, F3OMe, F3C, F3CON, F3CONdMe, 3Py6CON, Yac, YaeCOpipzaa, 5inda, F3aao, F3aa, F4aao, F4aa, 3Py6Nhae, 3Py6NHAc, 3Py6OMe, F4aaopipzaa, or F4amCOpipzaa;
   X9 is MeNal2, MeNal27N, MeF34diox, MeF34dOMe, MeF4T, MeY, MeW1Me, MeW7N, MeF3C₄Me, or MeF3Me4C;
   X10 is G, A, or a D-amino acid (e.g., da, ds, de, or dp);
   X11 is Bph, 3Py6Ph, F41Me4Pyz, F43Pyz, F44Pyz, F41Pyz, F41Me3Pyz, F4IEt4Pyz, F41MeOe4Pyz, F41MeOp4Pyz, F44thp, F4Ac4pip, PhNva, PhNle, Yph, Ybn, F4tb, F4oPr, or F4CONdMe; and
X12 is MeC; and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide. In some embodiments, the radiopharmaceutical conjugate comprises a radionuclide bound to the metal chelator. In some embodiments, the radiopharmaceutical conjugate comprises a covalently bound radionuclide.

In one aspect, described herein is a radiopharmaceutical conjugate comprising:
(a) a peptide that has avidity for Glypican 3 (GPC3), or a pharmaceutically acceptable salt thereof, wherein the peptide comprises an amino acid sequence of Formula (I),

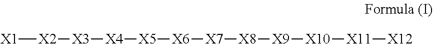

wherein,
   X1 is any amino acid;
   X2 is any amino acid;
   X3 is any amino acid;
   X4 is any amino acid;
   X5 is

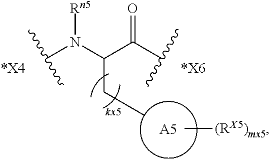

wherein:
   Rⁿ⁵ is hydrogen or C₁₋₃ alkyl:
   ring A5 is an aryl, heteroaryl, cycloalkyl, or heterocycloalkyl;
   each R^{X5} is independently C₁₋₆alkyl, C₁₋₆haloalkyl, C₁₋₆hydroxyalkyl, C₁₋₆aminoalkyl, C₁-(heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂NR^{c}R^{d}, —S(═O)(═NR^{a})R^{a}, —N═S(═O)NR^{c}R^{d}, —NR^{c}S(═O)₂R^{a}, amidinyl, —NR^{a}C(═NH)NR^{c}R^{d}, —NR^{a}S(═O)₂NR^{c}R^{d}, —C(═O)R^{a}, —C(═O)OR^{a}, —OC(═O)R^{d}, OC(═))OR^{a}, —OC(═O)NR^{c}R^{c}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X5}-heterocycloalkyl, -L^{X5}-cycloalkyl, -L^{X5}-aryl, or -L^{X5}-heteroaryl, wherein the alkyl, heteroalkyl, aryl, heteroaroyl, cycloalkyl, and heterocycloalkyl are optionally substituted with one or more R^{X5a}; or
   two R^{X5} are taken together to form ═O, ═S, or ═N(R^{a});
   L^{X5} is C₁₋₆alkylene, C₁₋₆heteroalkylene, —O—, —S—, or —NR^{a}—, wherein the alkylene and heteroalkylene is optionally substituted with one or more R^{X5a};
   kx5 is 0, 1, 2, or 3;
   mnx5 is 0, 1, 2, 3, 4, or 5;
   *X4 represents the point of attachment to X4; and
   *X6 represents the point of attachment to X6;
X6 is

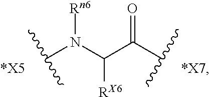

wherein;
   Rⁿ⁶ is hydrogen or C₁₋₃ alkyl;
   R^{X6} is C¹⁻⁶alkyl, C₁₋₆haloalkyl, C₁₋₆hydroxyalkyl, C₁₋₆aminoalkyl, C-heteroalkyl, C₂₋₆alkenyl, C₁₋₆alkynyl, aryl, heteroaryl, cycloalkyl, heterocycloalkyl, -L^{X6}-heterocycloalkyl, -L^{X6}-cycloalkyl, -L^{X6}-aryl, or -L^{X6}-heteroaryl, wherein each of the alkyl, heteroalkyl, alkenyl, alkynyl, aryl, heteroaryl, cycloalkyl, and heterocycloalkyl is optionally substituted with one or more R^{X6a}; or
   Rⁿ⁶ and R^{X6} are taken together with the intervening atoms to form a 5-to 6-membered heterocycloalkyl, which is optionally substituted with one or more R^{X6a};
   L^{X6} is C₁₋₆alkylene, C₁₋₆heteroalkylene, —O—, —S—, or —NR^{a}—, wherein the alkylene and heteroalkylene is optionally substituted with one or more R^{X6a};
   *X5 represents the point of attachment to X7; and
   *X7 represents the point of attachment to X7;
X7 is

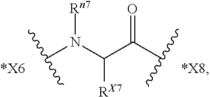

wherein:
   Rⁿ⁷ is hydrogen or C₁₋₃alkyl;
   R^{X7} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, cycloalkyl, or heterocycloalkyl, wherein each of the alkyl, heteroalkyl, alkenyl, alkynyl, cycloalkyl, and heterocycloalkyl is optionally substituted with one or more R^{X7a};
   *X6 represents the point of attachment to X6; and
   *X8 represents the point of attachment to X8;
X8 is

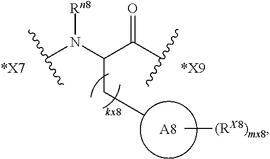

wherein;
   Rⁿ⁸ is hydrogen or C₁₋₃alkyl;
   ring A8 is an aryl or heteroaryl;
   each R^{X8} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SW, —SF₅, —NR^{c}R^{d}, —S(═O)R^{a}, —S(═O)₂R², —S(═O)₂R^{c}R^{d}, —S(═O)(═NR^{a})R^{a}, —N═S(═O)R^{c}R^{d}, —NR^{a}S(═O)₂R^{a} amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{d}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X8}-heterocycloalkyl, -L^{X8}-cycloalkyl, -L^{X8}-aryl, or -L^{X8}-heteroaryl, wherein each of the alkyl, heteroalkyl, alkenyl, alkynyl, aryl, heteroaryl, cycloalkyl, and heterocycloalkyl is optionally substituted with one or more R^{X8a}; or
   two R^{X8} are taken together to form ═O, ═S, or ═N(R^{a});
   L^{X8} is C₁₋₆alkylene, C₁₋₆heteroalkylene, —O—, —S—, or —NR^{a}—, wherein the alkylene and heteroalkylene is optionally substituted with one or more R^{X8a}
   kx8 is 0, 1, 2, or 3;
   mx8 is 0, 1, 2, 3, 4, or 5;
   *X7 represents the point of attachment to X7; and
   *X9 represents the point of attachment to X9.
X9 is

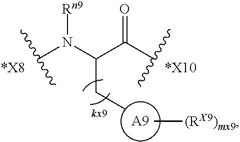

wherein:
   Rⁿ⁹ is hydrogen or C₁₋₃ alkyl;
   ring A9 is an aryl or heteroaryl;
   each R^{X9} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR², —SF₅, or —NR^{c}R^{d}, wherein each of the alkyl, heteroalkyl, alkenyl, and alkynyl is optionally substituted with one or more R_{X9a}; or
   two R^{X9} are taken together to form ═O, ═S, or ═N(R^{a});
   kx9 is 0, 1, 2, or 3;
   mx9 is 0, 1, 2, 3, 4, or 5;
   *X8 represents the point of attachment to X8, and
   *X10 represents the point of attachment to X10;
X10 is glycine or a D-amino acid (e.g., da, ds, de, or dp);
X11 is

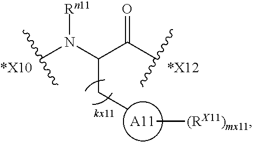

wherein:
   Rⁿ¹¹ is hydrogen or C₁₋₃alkyl;
   ring A11 is an aryl or heteroaryl;
   each R^{X11} is independently C₁₋₆alkyl, C₁₋₆haloalkyl, C₁₋₆ hydroxyalkyl, C₁₋₆aminoalkyl, C₁₋₆heteroalkyl, halogen, —CN, —NO₂, —OR^{a}, —SR^{a}, —SF₅, —NR^{c}R^{d}, —S(═O)R^{a}, —S(═O)₂R^{c}R^{d}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{d}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X11}-heterocycloalkyl, -L^{X11}-cycloalkyl, -L^{X11}-aryl, or -L^{X11}-heteroaryl, wherein each of the alkyl, heteroalkyl, aryl, heteroaryl, cycloalkyl, and heterocycloalkyl is optionally substituted with one or more R^{X11a}; or
   two R^{X11} are taken together to form ═O, ═S, or ═N(R^{a});
   L^{X11} is C₁₋₆alkylene. C₁₋₆heteroalkylene, —O—, —S—, or —NR²—, wherein the alkylene and heteroalkylene is optionally substituted with one or more R^{X11a};
   kx11 is 0, 1, 2, 3, 4, or 5;
   mx11 is 0, 1, 2, 3, 4, or 5;
   *X10 represents the point of attachment to X10; and,
   *X12 represents the point of attachment to X12;
X12 is N-alkylated cysteine;
each of R^{X5a}, R^{X6a}, R^{X7a}, R^{X8a}, R^{X9a} and R^{X11a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(O)OR^{a}, —NR^{a}C(═O)NR^{c}R^{d}, —C(O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, and alkynyl is optionally substituted with one or more R^{c}.
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e};
each R^{e} is independently halogen, —CN, —OH, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl, C₁-C₆haloalkyl, C₁-C₆hydroxyalkyl, C₁-C₆aminoalkyl, or C₁-C₆heteroalkyl; or two R^{e} are taken together to form ═O; and
each R^{c} and R^{d} are independently hydrogen, C₁-C₆alkyl, C₁-C₆haloalkyl, C₁-C₆hydroxyalkyl, C₁-C₅aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{c}; and
(b) (i) a metal chelator configured to bind with a radionuclide, wherein the metal chelator is bound to the peptide; or (ii) a covalently bound radionuclide.

In some embodiments, the radiopharmaceutical conjugate comprises a metal chelator configured to bind with a radionuclide. In some embodiments, the radiopharmaceutical conjugate comprises a radionuclide bound to the metal chelator. In some embodiments, the radiopharmaceutical conjugate comprises a covalently bound radionuclide.

In one aspect, described herein is a radiopharmaceutical conjugate with structure of

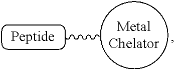

wherein [symbol] represents a linker.

In some embodiments, the radiopharmaceutical conjugate comprises a metal chelator configured to bind with a radionuclide, wherein the metal chelator is bound to the peptide. In some embodiments, the metal chelator is covalently linked to the peptide. In some embodiments, the conjugate further comprises a linker that connects the peptide with the metal chelator. In some embodiments, the linker covalently connects the peptide with the metal chelator. In some embodiments, the linker covalently attaches the metal chelator to the N-terminus of the peptide. In some embodiments, the linker covalently attaches the metal chelator to the C-terminus of the peptide. In some embodiments, the linker is attached to the peptide via a non-terminal amino acid residue of the peptide. In some embodiments, the linker is attached to amino acid X1. In some embodiments, the linker is attached to amino acid X2. In some embodiments, the linker is attached to amino acid X3. In some embodiments, the linker is attached to amino acid X4. In some embodiments, the linker is attached to amino acid X5. In some embodiments, the linker is attached to amino acid X6. In some embodiments, the linker is attached to amino acid X7. In some embodiments, the linker is attached to amino acid X8. In some embodiments, the linker is attached to amino acid X9. In some embodiments, the linker is attached to amino acid X10. In some embodiments, the linker is attached to amino acid X11. In some embodiments, the linker is attached to amino acid X1 2. In some embodiments, the linker is attached to amino acid X1, X2, X3, X4, X8, or X12. In some embodiments, the linker is attached to a lysine of the peptide. In some embodiments, the linker comprises one or more amino acid residues. In some embodiments, the linker comprises a lysine residue, an alanine residue, a glycine residue, a D-phenylalanine residue, a histidine residue, a dAtb residue, or a D-glutamate residue. In some embodiments, the linker comprises 2 amino acids selected from the group consisting of lysine, alanine, glycine, D-phenylalanine, histidine, dAtb, and D-glutamate. In some embodiments, the linker is a Cc heteroalkylene. In some embodiments, the linker is a bond.

In one aspect, described herein is a radiopharmaceutical conjugate with structure of

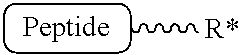

wherein [symbol] represents a linker; and
R* is a covalently bound radionuclide.

In some embodiments, the radiopharmaceutical conjugate comprises a covalently bound radionuclide. In some embodiments, the conjugate further comprises a linker that connects the peptide with the covalently bound radionuclide. In some embodiments, the linker covalently connects the peptide with the covalently bound radionuclide. In some embodiments, the linker covalently attaches the covalently bound radionuclide to the N-terminus of the peptide. In some embodiments, the linker covalently attaches the covalently bound radionuclide to the C-terminus of the peptide. In some embodiments, the linker is attached to the peptide via a non-terminal amino acid residue of the peptide. In some embodiments, the linker is attached to amino acid X1. In some embodiments, the linker is attached to amino acid X2. In some embodiments, the linker is attached to amino acid X3. In some embodiments, the linker is attached to amino acid X4. In some embodiments, the linker is attached to amino acid X5. In some embodiments, the linker is attached to amino acid X6. In some embodiments, the linker is attached to amino acid X7. In some embodiments, the linker is attached to amino acid X8. In some embodiments, the linker is attached to amino acid X9. In some embodiments, the linker is attached to amino acid X10. In some embodiments, the linker is attached to amino acid X11. In some embodiments, the linker is attached to amino acid X12. In some embodiments, the covalently bound radionuclide is directly covalently bound to an amino acid comprising an aromatic ring. In some embodiments, the covalent radionuclide is directly covalently bound to X5, X8, X9, X4, or X11. In some embodiments, the linker is a bond.

In some embodiments, described herein is a conjugate comprising: (a) a targeting moiety that comprises a monocyclic peptide that has avidity for Glypican 3 (GPC3), or a pharmaceutically acceptable salt thereof, and (b) (i) a metal chelator configured to bind with a radionuclide, wherein the metal chelator is bound to the peptide, or (ii) a covalently bound radionuclide. In some embodiments, described herein is a conjugate comprising: a monocyclic peptide that is configured to bind with GPC3 and a metal chelator configured to bind with a radionuclide. In some embodiments, described herein is a conjugate comprising: a monocyclic peptide that is configured to bind with GPC3 and a covalently bound radionuclide. In some embodiments, the monocyclic peptide is cyclized by a non-disulfide bond. In some embodiments, the monocyclic peptide does not comprise a disulfide bond. In some embodiments, the monocyclic peptide comprises 5 to 20 amino acid residues. In some embodiments, the monocyclic peptide comprises 7 to 14 amino acid residues. A conjugate described herein can further comprises a linker that covalently attaches the cyclic peptide to the metal chelator or covalent radionuclide. In some embodiments, the conjugate comprises a radionuclide such as ²²⁵Ac bound to the metal chelator, or ¹³¹I bound to the cyclic peptide . . . .

In some embodiments, a herein-described conjugate comprises two or more peptides (i.e., a first peptide, a second peptide, etc.). For example, the conjugate can comprise two different peptides, wherein both of the peptides are configured to bind to the same target (e.g., GPC3), either at the same binding site or at different binding sites. For another example, the conjugate can comprise two different peptides, wherein the two peptides are configured to bind to different targets (including GPC3). For yet another example, the conjugate can comprise two identical peptides.

In some embodiments, a herein-described conjugate is in a salt form. In some embodiments, a herein-described conjugate is in a free-base form.

In some embodiments, the metal chelator is conjugated to the peptide, either directly or indirectly through a linker. In some embodiments, the metal chelator is conjugated to the peptide, either covalently or non-covalently.

In some embodiments, a conjugate of the present disclosure is selected from Tables 2, 3A-3B and 9. In some embodiments, a conjugate of the present disclosure comprises a peptide of Table 1, a chelator selected from FIGS. 4-22, and a radionuclide of Table 7.

### Radiopharmaceutical Conjugate Having GPC3 Avidity

Glypican-3 (GPC3) is a protein that in humans is encoded by the GPC3 gene. GPC3 may be upregulated in multiple cancers, often correlating with disease progression, metastasis and poor prognosis e.g., in solid tumors such as hepatocellular, lung, gastric, and ovarian.

GPC3 belongs to the sulfate heparin proteoglycan family, and which is anchored on the cell membrane surface by phosphatidylinositol (GPI) anchor.

GPC3 can play an important role in the cell proliferation of embryo layer tissue. Deletion of GPC3 gene can cause excessive growth syndrome, namely Simpson-Golabi-Behmel syndrome (SGBS). GPC3 can be expressed throughout the entire fetal stage, and after birth to adult stage, except for placental, breast, mesodermal, ovarian, lung and kidney tissue with weak expression, other normal tissues have no obvious expression.

In some embodiments, the radiopharmaceutical conjugate of the present disclosure binds to GPC3. In some embodiments, the radiopharmaceutical conjugate has GPC3 antagonistic activity. In some embodiments, the radiopharmaceutical conjugate binds to human GPC3 (hGPC3) and has hGPC3 antagonistic activity, such as inhibiting the ability of GPC3 to promote or stabilize Wnt/Frizzled interaction and/or downstream signaling.

As used herein, the term “GPC3” refers to any form of GPC3 and a variant thereof for retaining at least a part of the activity of GPC3. The GPC3 includes all the native sequences of GPC3 in mammals such as, for example, humans, dogs, cats, horses, and cows, unless otherwise specifically described as human GPC3 (hGPC3). One exemplification of GPC3 is hGPC3 (Gene ID:2719), which is human GPC3 and is a protein having an amino acid sequence (SEQ ID NO: 398, Isoform 1, P51654-1):

MAGTVRTACLVVAMLLSLDFPGQAQPPPPPPDATCHQVRSFFQRLQPGLK
WVPETPVPGSDLQVCLPKGPTCCSRKMEEKYQLTARLNMEQLLQSASMEL
KFLIIQNAAVFQEAFEIVVRHAKNYTNAMFKNNYPSLTPQAFEFVGEFFT
DVSLYILGSDINVDDMVNELFDSLFPVIYTQLMNPGLPDSALDINECLRG
ARRDLKVFGNFPKLIMTQVSKSLQVTRIFLQALNLGIEVINTTDHLKFSK
DCGRMLTRMWYCSYCQGLMMVKPCGGYCNVVMQGCMAGVVEIDKYWREYI
LSLEELVNGMYRIYDMENVLLGLFSTIHDSIQYVQKNAGKLTTTIGKLCA
HSQQRQYRSAYYPEDLFIDKKVLKVAHVEHEETLSSRRRELIQKLKSFIS
FYSALPGYICSHSPVAENDTLCWNGQELVERYSQKAARNGMKNQFNLHEL
KMKGPEPVVSQIIDKLKHINQLLRTMSMPKGRVLDKNLDEEGFESGDCGD
DEDECIGGSGDGMIKVKNQLRFLAELAYDLDVDDAPGNSQQATPKDNEIS
TFHNLGNVHSPLKLLTSMAISVVCFFFLVH.

As used herein, the expression “has avidity for GPC3” or “binds to GPC3” indicates the activity of binding to GPC3. Binding site of the peptide of the present disclosure on the GPC3 is not limited, the peptide can bind to anywhere on the GPC3 protein. Binding to GPC3 may be measured by any method for measuring known intermolecular binding. In a non-limiting manner, for example, this may be determined by competitive binding assays such as surface plasmon resonance (SPR) assays, scatter analysis and/or radioimmunoassays (RIA), enzyme immunoassay s (EIA), and sandwich and competitive assays, and in any suitable manner which is known, including different variants of the examples given that are known in the technical field.

In one aspect, the binding affinity of the radiopharmaceutical conjugate of the present disclosure is at most 100 nM as determined by Kd in surface plasmon resonance (SPR) analysis. In some implementations, the Kd of the peptides of the radiopharmaceutical conjugates disclosed herein is 100 nM or less, 50 nM or less, 30 nM or less, 20 nM or less, 10 nM or less, 5 nM or less, 4 nM or less, 3 nM or less, 2 nM or less, 1 nM or less, 0.9 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 0.01 nM or less.

In some embodiments, a radiopharmaceutical conjugate described herein has a binding affinity to a human GPC3 of at most 1, 5, 10, 50, 100, 200, 500, 1000, 5000 or 10,000 nM as determined by Kd in surface plasmon resonance (SPR) analysis. In some embodiments, a radiopharmaceutical conjugate described herein has a binding affinity to a human GPC3 of at most 100 nM as determined by Kd in surface plasmon resonance (SPR) analysis. In some embodiments, a radiopharmaceutical conjugate described herein has a binding affinity to a human GPC3 of at most 1 nM as determined by Kd in surface plasmon resonance (SPR) analysis. In some embodiments, a radiopharmaceutical conjugate described herein has a binding affinity to a human GPC3 of at most 2 nM as determined by Kd in surface plasmon resonance (SPR) analysis. In some embodiments, a radiopharmaceutical conjugate described herein has a binding affinity to a human GPC3 of at most 5 nM as determined by Kd in surface plasmon resonance (SPR) analysis. In some embodiments, a radiopharmaceutical conjugate described herein has a binding affinity to a human GPC3 of at most 10 nM as determined by Kd in surface plasmon resonance (SPR) analysis.

In one aspect, the binding affinity of the peptide or radiopharmaceutical conjugate of the present disclosure is at most 100 nM as determined by Kd in surface plasmon resonance (SPR) analysis. In some embodiments, the Kd of the peptide or radiopharmaceutical conjugate of the present disclosure is 100 nM or less, 50 nM or less, 30 nM or less, 20 nM or less, 10 nM or less, 5 nM or less, 4 nM or less, 3 nM or less, 2 nM or less, 1 nM or less, 0.9 nM or less, 0.5 nM or less, 0.4 nM or less, 0.3 nM or less, 0.2 nM or less, 0.1 nM or less, 0.09 nM or less, 0.08 nM or less, 0.07 nM or less, 0.06 nM or less, 0.05 nM or less, 0.04 nM or less, 0.03 nM or less, 0.02 nM or less, 0.01 nM or less.

### GPC3 Peptide Binding Ligand

In one aspect, the disclosure relates to a radiopharmaceutical conjugate comprising a peptide (e.g., a binding peptide) that has avidity for Glypican-3 (GPC3). The GPC3 can be a mammalian GPC3. The GPC3 can be a human GPC3. The GPC3 can be a wild-type or mutated GPC3. In some embodiments, the conjugate of the disclosure comprises two or more peptides, which can be the same or different. The peptide can be linear or cyclic. In some embodiments, the peptide is monocyclic. The peptide can comprise any suitable number of amino acid residues. In some embodiments, the peptide comprises from 5 to 50, 6 to 40, 7 to 30, 8 to 25, 12 to 25, or 9 to 20 amino acid residues. In some embodiments, the peptide comprises from 5 to 14 amino acid residues. In some embodiments, the peptide comprises from 7 to 12 amino acid residues. In some embodiments, the peptide comprises from 8 to 12 amino acid residues. In some embodiments, the peptide comprises from 8 to 10 amino acid residues. In some embodiments, the peptide comprises from 7 to 13 amino acid residues. In some embodiments, the peptide comprises from 12 to 15 amino acid residues. In some embodiments, the peptide comprises from 13 to 14 amino acid residues. In some embodiments, the peptide comprises 6 amino acid residues. In some embodiments, the peptide comprises 7 amino acid residues. In some embodiments, the peptide comprises 8 amino acid residues. In some embodiments, the peptide comprises 9 amino acid residues. In some embodiments, the peptide comprises 10 amino acid residues. In some embodiments, the peptide comprises 11 amino acid residues. In some embodiments, the peptide comprises 12 amino acid residues. In some embodiments, the peptide comprises 13 amino acid residues. In some embodiments, the peptide comprises 14 amino acid residues. In some embodiments, the peptide comprises 15 amino acid residues. In some embodiments, the peptide comprises 16 amino acid residues. In some embodiments, the peptide consists of 6 amino acid residues. In some embodiments, the peptide consists of 7 amino acid residues. In some embodiments, the peptide consists of 8 amino acid residues. In some embodiments, the peptide consists of 9 amino acid residues. In some embodiments, the peptide consists of 10 amino acid residues. In some embodiments, the peptide consists of 11 amino acid residues. In some embodiments, the peptide consists of 12 amino acid residues. In some embodiments, the peptide consists of 13 amino acid residues. In some embodiments, the peptide consists of 14 amino acid residues. In some embodiments, the peptide consists of 15 amino acid residues. In some embodiments, the peptide consists of 16 amino acid residues, in some embodiments, the conjugate comprises a monocyclic peptide of 6, 7, 8, 9, 10, 11, 12, 13, 14, or 15 amino acid residues. A peptide described herein can be a binding peptide that binds to GPC3. In some embodiments, the binding peptide consists of 6 to 20 amino acid residues. In some embodiments, the binding peptide consists of 7 to 12 amino acid residues. In some embodiments, the binding peptide consists of 10 to 12 amino acid residues. In some embodiments, the binding peptide consists of 8 to 12 amino acid residues. In some embodiments, the binding peptide is monocyclic. In some embodiments, the peptide of the present technology is an isolated peptide. In some embodiments, the peptide of the present technology is a purified peptide.

In one aspect, described herein is a radiopharmaceutical conjugate comprising:
(a) a peptide that has avidity for Glypican 3 (GPC3), or a pharmaceutically acceptable salt thereof, wherein the peptide comprises an amino acid sequence including deletion, substitution, and/or addition of one or several (e.g., 1-6) amino acids in the amino acid sequence of SEQ ID NO: 1:

(SEQ ID NO: 1)
MeK-MeI-D-MeQ-F4COO-I-I-Y-MeNal27N-G-3Py6Ph-MeC,

wherein the peptide consists of 10 to 12 amino acid residues; and
(b) (i) a metal chelator configured to bind with a radionuclide, wherein the metal chelator is bound to the peptide: or (ii) a covalently bound radionuclide.

In some embodiments, the metal chelator is configured to bind with a radionuclide, wherein the metal chelator is bound to the peptide. In some embodiments, the radionuclide is bound to the metal chelator. In some embodiments, the radionuclide is a covalently bound radionuclide.

In some embodiments, the peptide comprises an amino acid sequence including a total of at most 6 deletion, substitution, and/or addition of one or several amino acids in the amino acid of SEQ ID NO:1. In some embodiments, the peptide comprises an amino acid sequence including a total of at most 5 deletion, substitution, and/or addition of one or several amino acids in the amino acid of SEQ ID NO:1. In some embodiments, the peptide comprises an amino acid sequence including a total of at most 4 deletion, substitution, and/or addition of one or several amino acids in the amino acid of SEQ ID NO:1. In some embodiments, the peptide comprises an amino acid sequence including a total of at most 3 deletion, substitution, and/or addition of one or several amino acids in the amino acid of SEQ ID NO:1. In some embodiments, the peptide comprises an amino acid sequence including a total of at most 2 deletion, substitution, and/or addition of one or several amino acids in the amino acid of SEQ ID NO:1. In some embodiments, the peptide comprises an amino acid sequence including a total of at most 1 deletion, substitution, and/or addition of one or several amino acids in the amino acid of SEQ ID NO:1, In some embodiments, the amino acid substitution is a conservative amino acid substitution. The deletion, addition, or substitution position may be either the end or middle of the peptide. In some embodiments, the MeI at position 2 is deleted from SEQ ID NO: 1. In some embodiments, the D at position 3 is deleted from SEQ ID NO: 1. In some embodiments, the peptide of SEQ ID NO: 1 is a cyclic peptide. In some embodiments, the peptide is a monocyclic peptide. In some embodiments, 1, 2, 3, 4 or 5 amino acids are added to the peptide of SEQ ID NO: 1. In some embodiments, 1, 2, or 3 amino acids are added to the peptide of SEQ ID NO: 1. In some embodiments, 1 or 2 amino acids are added to the peptide of SEQ ID NO: 1. In some embodiments, 5 amino acids are added to the peptide of SEQ ID NO: 1. In some embodiments, 4 amino acids are added to the peptide of SEQ ID NO: 1 In some embodiments, 3 amino acids are added to the peptide of SEQ ID NO: 1 In some embodiments, 2 amino acids are added to the peptide of SEQ ID NO: 1 In some embodiments, 1 amino acids is added to the peptide of SEQ ID NO: 1 The amino acid addition may be at either the N-terminus, the C-terminus, or the middle of the peptide of SEQ ID NO: 1.

In one aspect, described herein is a peptide that has avidity for Glypican 3 (GPC3), or a pharmaceutically acceptable salt thereof, wherein the peptide comprises an amino acid sequence of Formula (I):

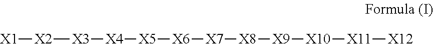

wherein,
   X1 is any amino acid;
   X2 is absent or any amino acid;
   X3 is absent or any amino acid;
   X4 is any amino acid;
   X5 is an amino acid comprising an aromatic ring (e.g., W, F, Y, or a variant thereof), cycloalkyl, or heterocycloalkyl group, or X5 is a peptoid (e.g., Cha4cH, Cha4tH, A1mor, Atp, Cha4cOMe);
   X6 is a hydrophobic amino acid, a hydrophilic amino acid, or a polar amino acid wherein the polar amino acid has a substituted side chain;
   X7 is a hydrophobic amino acid comprising a C₁-C₈ alkyl, cycloalkyl, or heterocycloalkyl, wherein the alkyl, cycloalkyl, and heterocycloalkyl are each independently, optionally substituted (e.g., X7 is I, Eva, alI, TMe, SMe, Gcpr, Gcpe, Gthp, dMeS, TdMe. or Cbg);
   X8 is an A, 1, L, V, Y or F, or a variant thereof;
   X9 is an N-alkylated amino acid comprising an aromatic ring;
   X10 is G, A, or a D-amino acid (e.g., da, ds, de, or dp);
   X11 is an amino acid comprising an aromatic ring (e.g., F, Y, or a variant thereof); and,
   X12 is N-alkylated cysteine (e.g., MeC).

In some embodiments, the peptide of Formula (I) is a cyclic peptide. In some embodiments, the peptide is a monocyclic peptide.

In some embodiments, a peptide of Formula (I) is a cyclic peptide. In some embodiments, the cyclic peptide of Formula (I) has the structure of Formula (I-1):

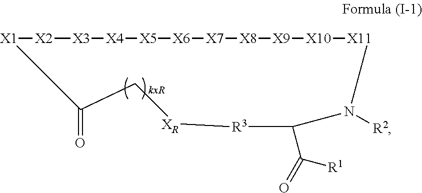

wherein,
   R¹ is selected from the group consisting of —NH₂ and —OH;
   R² is C₁₋₃ alkyl;
   R¹ is C₁₋₃ alkylene, optionally substituted with one or more R⁴;
   each R⁴ is independently C₁₋₃ alkyl or C₃₋₆ cycloalkyl;
   kxR is 1, 2, 3, 4, 5, or 6;
   X_{R} is selected from the group consisting of —S—, —CH₂—, or —O—; and
wherein X1 to X11 have the definitions described in Formula (I).

In some embodiments, a peptide of Formula (I-1) has the structure of Formula (I-2):

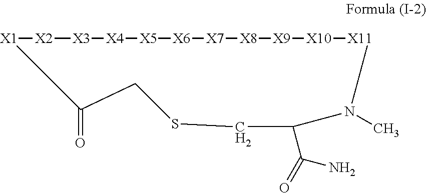

wherein X1 to X11 have the definitions described in Formula (I).

In some embodiments, a peptide of Formula (I) is a cyclic peptide and X2 is absent. In some embodiments, the cyclic peptide of Formula (I) has the structure of Formula (I-3):

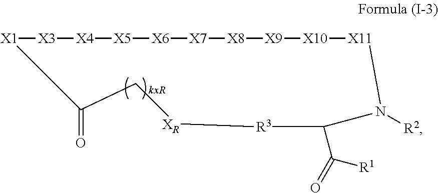

wherein,
   R¹ is selected from the group consisting of —NH₂ and —OH;
   R² is C₁₋₃ alkyl;
   R³ is C₁₋₃alkylene, optionally substituted with one or more R⁴;
   each R⁴ is independently C₁₋₃ alkyl or C₃₋₆ cycloalkyl;
   kxR is 1, 2, 3, 4, 5, or 6;
   X_{R} is selected from the group consisting of —S—, —CH₂—, or —O—; and
wherein X1 to X11 have the definitions described in Formula (I).

In some embodiments, a peptide of Formula (I-3) has the structure of Formula (I-4):

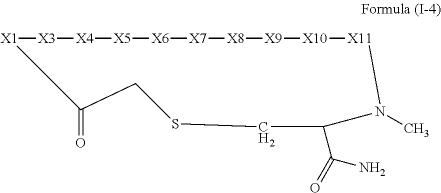

wherein X1 to X11 have the definitions described in Formula (I).

In some embodiments, the peptide of Formula (I) has a structure of Formula (I-5), or a pharmaceutically acceptable salt thereof:

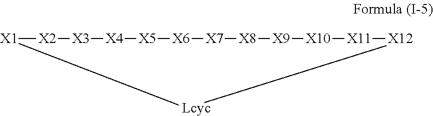

wherein X1-X12 have the definition described for Formula (I) and Lcyc is a ring closing group that covalently connecting X1 with X12. In some embodiments, the Lcyc is a group selected from Table 4B. In some embodiments, the Lcyc is formed by reacting the first and the second functional groups in Table 4C.

In one aspect, described herein is a radiopharmaceutical conjugate comprising:
a peptide that has avidity for Glypican 3 (GPC3), or a pharmaceutically acceptable salt thereof, wherein the peptide comprises an amino acid sequence of Formula (I):

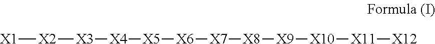

wherein,
   X1 is any amino acid;
   X2 is any amino acid;
   X3 is any amino acid;
   X4 is any amino acid;
   X5 is

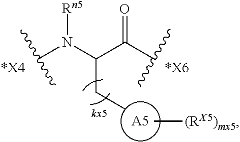

wherein:
   Rⁿ⁵ is hydrogen or C₁₋₃ alkyl;
   ring A5 is an aryl, heteroaryl, cycloalkyl, or heterocycloalkyl;
   each R^{X5} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂NR^{c}R^{a}, —S(═O)(═NR^{a})R^{a}, —N═S(═O)NR^{c}R^{d}, —NR^{a}S(═O)₂R^{a} amidinyl, —NR^{a}C(═NH)NR^{c}R^{d}, —NR^{a}S(═O)₂NR^{c}R^{d}, —C(═O)R^{a}, —C(═O)OR^{a}, —OC(═O)R^{d}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{a}), —P(═O)R^{c}R^{d}, aryl, heteroaryl, cycloalkyl, heterocycloalkyl, -L^{X5}-heterocycloalkyl, -L^{X5}-cycloalkyl, -L^{X5}-aryl, or -L^{X5}-heteroaryl, wherein the alkyl, heteroalkyl, aryl, heteroaryl, cycloalkyl, and heterocycloalkyl are optionally substituted with one or more R^{X5a}; or
   two R^{X5} are taken together to form ═O, ═S, or ═N(R^{a});
   L^{X5} is C₁₋₆alkylene, C₁₋₆heteroalkylene, —O—, —S—, or —NR^{a}—, wherein the alkylene and heteroalkylene is optionally substituted with one or more R^{X5};
   kx5 is 0, 1, 2, or 3;
   mx5 is 0, 1, 2, 3, 4, or 5;
   *X4 represents the point of attachment to X4; and
   *X6 represents the point of attachment to X6;
X6 is

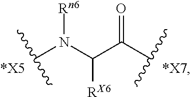

wherein;
   Rⁿ⁶ is hydrogen or C₁₋₃ alkyl;
   R^{X6} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X6}-heterocycloalkyl, -L^{X6}-cycloalkyl, -L^{X6}-aryl, or -L^{X6}-heteroaryl, wherein each of the alkyl, heteroalkyl, alkenyl, alkynyl, aryl, heteroaryl, cycloalkyl, and heterocycloalkyl is optionally substituted with one or more R^{X6a}; or
   Rⁿ⁶ and R^{X6} are taken together with the intervening atoms to form a 5-to 6-membered heterocycloalkyl, which is optionally substituted with one or more R^{X6};
   L^{X6} is C₁₋₆alkylene, C₁₋₆heteroalkylene, —O—, —S— or —NR^{a}—, wherein the alkylene and heteroalkylene is optionally substituted with one or more R^{X6a};
   *X5 represents the point of attachment to X5; and
   *X7 represents the point of attachment to X7:
X7 is

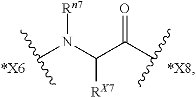

wherein:
   Rⁿ⁷ is hydrogen or C₁₋₃ alkyl;
   R^{X7} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, cycloalkyl, or heterocycloalkyl, wherein each of the alkyl, heteroalkyl, alkenyl, alkynyl, cycloalkyl, and heterocycloalkyl is optionally substituted with one or more R^{X7a};
   *X6 represents the point of attachment to X6; and
   *X8 represents the point of attachment to XX;
X8 is

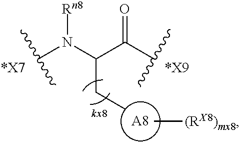

wherein;
   Rⁿ⁸ is hydrogen or C₁₋₃alkyl;
   ring A8 is an aryl or heteroaryl;
   each R^{X8} is independently C₁₋₆alkyl, C₁₋₆haloalkyl, C₁₋₆hydroxy alkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{c}, —N═S(═O)R^{c}R^{d}, —NR^{a}S(═O)₂R^{a}, amidinyl, —NR^{a}C(═NH)NR^{c}R^{a}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X8}-heterocycloalkyl, -L^{X8}-cycloalkyl, -L^{X8}-aryl, or -L^{X8}-heteroaryl wherein each of the alkyl, heteroalkyl, alkenyl, alkynyl, aryl, heteroaroyl, cycloalkyl, and heterocycloalkyl is optionally substituted with one or more R^{X8a}; or
   two R^{X8} are taken together to form ═O, ═S, or ═N(R^{a});
   L^{X8} is C₁₋₆alkylene, C₁₋₆heteroalkylene, —O—, —S—, or —NR^{a}—, wherein the alkylene and heteroalkylene is optionally substituted with one or more R^{X8a};
   kx8 is 0, 1, 2, or 3;
   mx8 is 0, 1, 2, 3, 4, or 5;
   *X7 represents the point of attachment to X7; and
   *X9 represents the point of attachment to X9;
X9 is

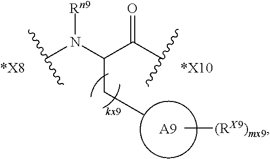

wherein:
   Rⁿ⁹ is hydrogen or C₁₋₃alkyl:
   ring A9 is an aryl or heteroaryl:
   each R^{X9} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, or —NR^{c}R^{d}, wherein each of the alkyl, heteroalkyl, alkenyl, and alkynyl is optionally substituted with one or more R^{X9}; or
   two R^{X9} are taken together to form ═O, ═S, or ═N(R^{a});
   kx9 is 0, 1, 2, or 3;
   mx9 is 0, 1, 2, 3, 4, or 5;
   *X8 represents the point of attachment to X8; and
   *X10 represents the point of attachment to X10;
X10 is glycine or a D-amino acid (e.g., da, ds, de, or dp);
X11 is

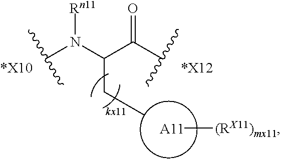

wherein:
   Rⁿ¹¹ is hydrogen or C₁₋₃ alkyl;
   ring A11 is an aryl or heteroaryl;
   each R^{X11} is independently C₁₋₆alkyl, C₁₋₆haloalkyl, C₁₋₆hydroxyalkyl, C₁₋₆aminoalkyl, C₁₋₆heteroalkyl halogen, —CN, —NO₂, —OR^{a}, —SR^{a}, —SF₅, —NR^{c}R^{d}, —S(═O)R^{a}, —S(═O)₂R, —S(═O)₂R^{c}R^{a}, —S(═O)(═NR)R^{a}, —N═S(═O)R^{c}R^{d}, —NRₐS(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X11}-heterocycloalkyl, -L^{X11}-cycloalkyl, -L^{X11}-aryl, or -L^{X11}-heteroaryl, wherein each of the alkyl, heteroalkyl, aryl, heteroaryl, cycloalkyl, and heterocycloalkyl is optionally substituted with one or more R^{X11a}; or
   two R^{X11} are taken together to form ═O, ═S, or ═N(R^{a});
   L^{X11} is C₁₋₆alkylene, C₁₋₆heteroalkylene, —O—, —S—, or —NR^{a}—, wherein the alkylene and heteroalkylene is optionally substituted with one or more R^{X11a};
   kx11 is 0, 1, 2, 3, 4, or 5;
   mx11 is 0, 1, 2, 3, 4, or 5;
   *X10 represents the point of attachment to X10; and,
   *X12 represents the point of attachment to X12;
X12 is N-alkylated cysteine:
each of R^{X5a}, R^{X6a}, R^{X7a}, R^{X8a}, R^{X9a}, and R^{X11a} is independently halogen, C₁₋₆ alkyl, C₁-C₆haloalkyl, C₁-C₆ hydroxyalkyl, C₁-C₆aminoalkyl, C₁-C₆heteroalkyl, C₂-C₆alkenyl, C₂-C₆alkynyl, —CN, —NO₂, —OR), —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O),NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, and alkynyl is optionally substituted with one or more R^{e}.
each R^{a} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e};
each R^{e} is independently halogen, —CN, —OH, —O—C₁-C₆alkyl, —SF₅, —S(═O)C₁-C₆alkyl, —S(═O)₂C₁-C₆alkyl, —S(═O)₂NH₂, —S(═O)₂-halogen, —S(═O)₂NHC₁-C₆alkyl, —S(═O)₂N(C₁-C₆alkyl)₂, —NH₂, —NHC₁-C₆alkyl, —N(C₁-C₆alkyl)₂, —NHC(═NH)NH₂, —NHC(═O)OC₁-C₆alkyl, —C(═O)C₁-C₆alkyl, —C(═O)OH, C₁-C₆alkyl-C(═O)OH, —C(═O)OC₁-C₆alkyl, —C(═O)NH₂, —C(═O)N(C₁-C₆alkyl)₂, —C(═O)NHC₁-C₆alkyl. C₁-C₆haloalkyl, C₁-C₆hydroxyalkyl, C₁-C₆aminoalkyl, or C₁-C₆heteroalkyl; or two R^{e} are taken together to form ═O; and
each R^{c} and R^{d} are independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, heteroaryl, C₁-C₆alkyl(cycloalkyl), C₁-C₆alkyl(heterocycloalkyl), C₁-C₆alkyl(aryl), or C₁-C₆alkyl(heteroaryl), wherein each of the alkyl, alkenyl, alkynyl, cycloalkyl, heterocycloalkyl, aryl, and heteroaryl is independently optionally substituted with one or more R^{e}; or R^{c} and R^{d} are taken together with the atom to which they are attached to form a heterocycloalkyl optionally substituted with one or more R^{e}.

In some embodiments of Formula (I), (I-1), (I-2), (I-3), (I-4), (I-5), (III-1), (III-2), (IV-1), or (IV-2), X1 is an N-methylated amino acid. In some embodiments X1 is an N-methylated amino acid comprising a polar side chain (e.g., MeK, MeQ, or a variant thereof).

In some embodiments of Formula (I), (I-1), (I-2), (I-3), (I-4), (I-5), (III-1), (III-2), (IV-1), or (IV-2), X1 is I, R, Cit, F4G, 4Py, 3Py, KCOpipzaa, V, Eva, Q, E, MeI, Ahp, F4COO, KCOpip4COO, MeQdMe, MeA, MeSMe, MeG, MeV. MeHseMe, Aib, MeT, alI, TMe. MeKCOpipzaa, MeQ, Hpr, MeTMe. MeDapCOpipzaa, MeK. MeKAc. MeK(de), MeK(H), MeK(df), or MeK(datb). In some embodiments of Formula (I), (I-1), (I-2), (I-3), (I-4), (I-5), (III-1), (III-2), (IV-1), or (IV-2), X1 is 1, R, Cit, F4G, 4Py, 3Py, KCOpipzaa, V, Eva, Q, E, MeI, Ahp, F4COO, KCOpip4COO, MeQdMe, MeA, MeSMe, MeG, MeV. MeHseMe, Aib, MeT, alI, TMe, MeKCOpipzaa, MeQ, Hpr, MeTMe, MeDapCOpipzaa, MeK, MeKAc, MeK(de), MeK(1H), MeK(df), or MeK(datb), each of which is optionally substituted. In some embodiments, X1 is optionally substituted L In some embodiments, X1 is optionally substituted R. In some embodiments, X1 is optionally substituted Cit. In some embodiments, X1 is optionally substituted F4G. In some embodiments, X1 is optionally substituted 4Py. In some embodiments, X1 is optionally substituted 3Py. In some embodiments, X1 is optionally substituted KCOpipzaa. In some embodiments, X1 is optionally substituted V. In some embodiments, X1 is optionally substituted Eva. In some embodiments, X1 is optionally substituted Q. In some embodiments, X1 is optionally substituted E. In some embodiments, X1 is optionally substituted MeI. In some embodiments, X1 is optionally substituted Alip. In some embodiments, X1 is optionally substituted F4COO. In some embodiments, X1 is optionally substituted KCOpip4COO. In some embodiments, X1 is optionally substituted MeQdMe. In some embodiments, X1 is optionally substituted MeA. In some embodiments, X1 is optionally substituted MeSMe. In some embodiments, X1 is optionally substituted MeG. In some embodiments, X1 is optionally substituted MeV. In some embodiments, X1 is optionally substituted MeHseMe. In some embodiments. X1 is optionally substituted Aib. In some embodiments, X1 is optionally substituted MeT. In some embodiments, X1 is optionally substituted all. In some embodiments, X1 is optionally substituted TMe. In some embodiments. X1 is optionally substituted MeKCOpipzaa. In some embodiments. X1 is optionally substituted MeQ. In some embodiments, X1 is optionally substituted Hpr. In some embodiments, X1 is optionally substituted MeTMe. In some embodiments, X1 is optionally substituted MeDapCOpipzaa. In some embodiments, X1 is optionally substituted MeK. In some embodiments, X1 is optionally substituted MeKAc. In some embodiments, X1 is optionally substituted MeK(de). In some embodiments, X1 is optionally substituted MeK(H). In some embodiments, X1 is optionally substituted MeK(df). In some embodiments, X1 is optionally substituted MeK(datb). In some embodiments, X1 is MeQ or a derivative thereof. In some embodiments, X1 is MeK or a derivative thereof.

In some embodiments of a peptide of Formula (I), (I-1), (I-2), (I-3), (I-4), (I-5), (II-1), (III-2), (IV-1), or (IV-2), X1 is any amino acid. In some embodiments, X1 is a D-amino acid. In some embodiments, X1 is an L-amino acid. In some embodiments, X1 is an N-alkylated amino acid . . . . In some embodiments, X1 is an N-methylated amino acid. In some embodiments, X1 does not comprise a cyclic group. In some embodiments, X1 comprises a cyclic group. In some embodiments, X1 comprises a 5-6 membered heterocycloalkyl group. In some embodiments, X1 comprises a 5-6 membered heteroaryl group. In some embodiments, X1 comprises a phenyl group. In some embodiments, X1 is a peptoid.

In some embodiments of a peptide of Formula (I), (I-1), (I-2), (I-3), (I-4), (I-5), (I-1), (III-2), (IV-1), or (IV-2), X1 has a structure of

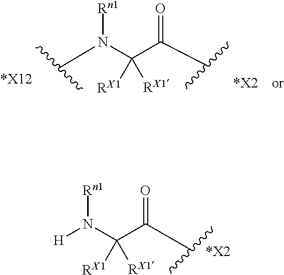

wherein:
   Rⁿ¹ is hydrogen or C₁₋₃ alkyl;
   R^{X1} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X1}-heterocycloalkyl, -L^{X1}-cycloalkyl, -L^{X1}-aryl, or -L^{X1}-heteroaryl, wherein each of the alkyl, alkenyl, alkynyl, heteroalkyl, aryl, heteroaryl, cycloalkyl and heterocycloalkyl is optionally substituted with one or more R^{X1a};
   R^{X1′} is hydrogen or C₁₋₆alkyl, wherein the alkyl is optionally substituted with one or more R^{X1a}; or
   Rⁿ¹ and R^{X1′} are taken together with the intervening atoms to form a 5-to 6-membered heterocycloalkyl, which is optionally substituted with one or more R^{X1a};
   L^{X1} is C₁₋₆alkylene, C₁₋₆heteroalkylene, —O—, —S—, or —NR^{a}—, wherein the alkylene and heteroalkylene is optionally substituted with one or more R^{X1a}
   each R^{X1a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R², —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR)(OR^{d}), —P(═O)R^{c}R^{d}, aryl, heteroaryl, cycloalkyl, heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
   *X12 represents the point of attachment to X12, or *X12 represents the point of attachment to C(O)(CH₂)_{kxR} (e.g., in Formula (I-1) and (I-2));
   *X2 represents the point of attachment to X2; and other groups (such as R^{a}, R^{c}, R^{d}, and R^{e}) have the meanings defined in Formula (I).

In some embodiments, X1 has a structure of

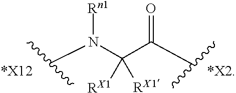

In some embodiments, X1 has a structure of

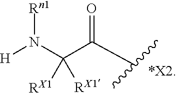

In some embodiments of a peptide of Formula (I), (I-1), (I-2), (I-3), (I-4), (I-5), (III-1), (III-2), (IV-1), or (IV-2), X1 has a structure of

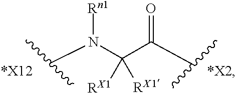

wherein:
   Rⁿ¹ is hydrogen or methyl;
   R^{X1} is hydrogen, C₁₋₆alkyl, C₁₋₆haloalkyl, C₁₋₆hydroxyalkyl, C₁₋₆aminoalkyl, C₁₋₆heteroalkyl, -L^{X1}-5-6 membered heterocycloalkyl, -L^{X1}-C-cycloalkyl, -L^{X1}-C₆₋₁₀aryl, or -L^{X1}-5-10 membered heteroaryl, wherein each of the alkyl, heteroalkyl, aryl, heteroaryl, cycloalkyl, and heterocycloalkyl is optionally substituted with one or more R^{X1a};
   R^{X1′} is hydrogen or methyl; or
   Rⁿ¹ and R^{X1′} are taken together with the intervening atoms to form a 5-to 6-membered heterocycloalkyl, which is optionally substituted with one or more R^{X1a};
   L^{X1} is C₁₋₆alkylene or C₁₋₆heteroalkylene, wherein the alkylene and heteroalkylene is optionally substituted with one or more R^{X1a};
   each R^{X1a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO^{a}, —OR^{a}, —SR^{a}, —NR^{c}R^{d}, —S(═OR^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a}), —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
   *X12 represents the point of attachment to X12, or *X12 represents the point of attachment to C(O)(CH₂)_{kxR} (e.g., in Formula (I-1) and (I-2);
   *X2 represents the point of attachment to X2; and other groups (such as R^{a}, R^{c}, R^{d}, and R^{e}) have the meanings defined in Formula (I).

In some embodiments, Rⁿ¹ is hydrogen. In some embodiments, Rⁿ¹ is C₁₋₃ alkyl. In some embodiments, Rⁿ¹ is methyl.

In some embodiments, R^{X1} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X1}-heterocycloalkyl, -L^{X1}-cycloalkyl, -L^{X1}-aryl, or -L^{X1}-heteroaryl, wherein each of the alkyl, alkenyl, alkynyl, heteroalkyl, aryl, heteroaryl, cycloalkyl, and heterocycloalkyl is optionally substituted with one or more R^{X1a}.

In some embodiments, R^{X1} is hydrogen, C₁₋₆alkyl, C₁₋₆haloalkyl, C₁₋₆hydroxyalkyl, C₁₋₆aminoalkyl, C₁₋₆heteroalkyl, -L^{X1}-5-6 membered heterocycloalkyl, -L^{X1}-C₄₋₆cycloalkyl, -L^{X1}-C₁₋₆aryl, or -L^{X1}-5-10 membered heteroaryl, wherein each of the alkyl, heteroalkyl, aryl, heteroaryl, cycloalkyl, and heterocycloalkyl is optionally substituted with one or more R^{X1a}. In some embodiments, R^{X1} is hydrogen, C₁₋₆alkyl, C₁₋₆heteroalkyl, -L^{X1}-piperidinyl, -L^{X1}-piperazinyl, -L^{X1}-phenyl, or -L^{X1}-pyridinyl, wherein each of the alkyl, heteroalkyl, phenyl, pyridinyl, piperidinyl, and piperazinyl is optionally substituted with one or more R^{X1a}.

In some embodiments, R^{X1} is hydrogen. In some embodiments, R^{X1} is optionally substituted C₁₋₆alkyl. In some embodiments, R^{X1} is C₁₋₆alkyl optionally substituted with one or more R^{X1a}. In some embodiments, R^{X1} is optionally substituted C₁₋₆heteroalkyl. In some embodiments, R^{X1} is C₁₋₆heteroalkyl optionally substituted with one or more R^{X1a}. In some embodiments, R^{X1} is C₁₋₆alkyl, C₁₋₆haloalkyl, C₁₋₆hydroxyalkyl, C₁₋₆aminoalkyl, or C₁₋₆heteroalkyl, wherein each of which is optionally substituted with one or more R^{X1}. In some embodiments, R^{X1} is C₁₋₆aminoalkyl. In some embodiments, R^{X1} is —(CH)₁₋₆NH₂. In some embodiments, R^{X1} is —(CH)₄NH₂. In some embodiments, R^{X1a} is C₂₋₆alkenyl or C₂₋₆alkynyl, wherein each of which is optionally substituted with one or more R^{X1a}. In some embodiments, R^{X1} is aryl, heteroaryl, cycloalkyl, or heterocycloalkyl, wherein each of which is optionally substituted with one or more R^{X1a}. In some embodiments, R^{X1} is aryl or heteroaryl, wherein each of which is optionally substituted with one or more R^{X1a}. In some embodiments, R^{X1} is cycloalkyl or heterocycloalkyl, wherein each of which is optionally substituted with one or more R^{X1a}. In some embodiments. R^{X1} is -L^{X5}-heterocycloalkyl, -L^{X1}-cycloalkyl, -L^{X1}-aryl, or -L^{X1}-heteroaryl, wherein each of which is optionally substituted with one or more R^{X1a}. In some embodiments, R^{X1} is -L^{X5}-heterocycloalkyl, which is optionally substituted with one or more R^{X1a}. In some embodiments, R^{X1} is -L^{X1}-cycloalkyl, which is optionally substituted with one or more R^{X1a}. In some embodiments, R^{X1} is -L^{X1}-aryl, which is optionally substituted with one or more R^{X1a}. In some embodiments, R^{X1} is -L^{X1}-heteroaryl, which is optionally substituted with one or more R^{X1a}.

In some embodiments, R^{X11} is hydrogen. In some embodiments, R^{X1′} is C₁₋₆alkyl, wherein the alkyl is optionally substituted with one or more R^{X1a}. In some embodiments, R^{X1′} is C₁₋₆alkyl. In some embodiments, R^{X1′} is methyl.

In some embodiments, Rⁿ¹ and R^{X1′} are taken together with the intervening atoms to form a 5-to 6-membered heterocycloalkyl, which is optionally substituted with one or more R^{X1a}.

In some embodiments, L^{X1} is C₁₋₆alkylene, wherein the alkylene is optionally substituted with one or more R^{X1a}. In some embodiments, L^{X1} is C₁₋₆alkylene. In some embodiments, L^{X1} is C₁₋₆ heteroalkylene, wherein the heteroalkylene is optionally substituted with one or more R^{X1a}. In some embodiments, L^{X1} is C₁₋₆heteroalkylene. In some embodiments, L^{X1} is —O—. In some embodiments, L^{X1} is —S—. In some embodiments, L^{X1} is —NR^{a}—. In some embodiments, L^{X1} is —NH—.

In some embodiments, each R^{X1a} is independently halogen. C₁₋₆ alkyl, C₁-C₆haloalkyl, C₁-C₆hydroxyalkyl, C₁-C₆aminoalkyl, C₁-C₆heteroalkyl, cycloalkyl, heterocycloalkyl, —CN, —NO₂, —OR^{a}, —SR₅, —NR^{c}R^{d}, —S(═O)R^{a}, —S(═O)₂R^{a}, —S(═O)₂NR^{c}R^{d}, —NR^{a}S(═O)₂R, —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR²C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X1a} is independently halogen, C₁₋₆ alkyl, C₁₋₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{c}, —NR^{c}C(═O)NR^{c}R^{d}, —C(═O)NR^{c}R^{d}, or ═O, wherein each of the alkyl and heteroalkyl is optionally substituted with one or more R^{e}.

In some embodiments, each R^{X1a} is independently C₁₋₆alkyl, C₁₋₆ alkoxyl, C₁-C₆haloalkyl, C₁-C₆hydroxyalkyl, C₁-C₆aminoalkyl, OH, —CN, —NO₂, —NH₂, —C(═O)R^{a}, —C(═O)OR^{a}, —NR^{a}C(═O)NR^{c}R^{d}, or oxo.

In some embodiments, Rⁿ¹ is methyl, R^{X1′} is hydrogen, and R^{X1} is C₁₋₆aminoalkyl. In some embodiments, R^{X1′} is methyl, R^{X1} is hydrogen, and R^{X1} is C₁₋₆aminoalkyl. In some embodiments, Rⁿ¹ is methyl, R^{X1′} is hydrogen, and R^{X1} is C₁₋₃alkyl, which the alkyl is substituted with an amino.

In some embodiments of Formula (I), (I-1), (I-2), (I-3), (I-4), (I-5), (III-1), (III-2), (IV-1), or (IV-2), X2 is absent or an L-amino acid. In some embodiments, X2 is absent. In some embodiments, X2 is an L-amino acid. In some embodiments, X2 is a N-methylated amino acid.

In some embodiments of Formula (I), (I-1), (I-2), (I-3), (I-4), (I-5), (III-1), (III-2), (IV-1), or (IV-2), X2 is I, K, Cit, F4G, 4Py, 3Py, KCOpipzetOH, V, KCOpipzaa, Eva, Q, E, S, Ahp, F4COO, KCOpip4COO, MeQdMe, MeA, MeSMe, MeG, MeI, MeV, MeL, HseMe, MeY, Me3Py, MeHseMe, MeKAc, alI, TMe MeKCOpipzaa, MeQ, Hpr, MeTMe, or MeDapCOpipzaaa. In some embodiments of Formula (I), (I-1), (I-2), (I-3), (I-4), (I-5), (III-1), (III-2), (IV-1), or (IV-2), X2 is I, K, Cit, F4G, 4Py, 3Py, KCOpipzetOH, V. KCOpipzaa, Eva, Q, E, Ahp, F4COO, KCOpip4COO, MeQdMe, MeA, MeSMe, MeG, MeI, MeV, MeHseMe, MeKAc, alI, TMe MeKCOpipzaa, MeQ, Hpr, MeTMe, or MeDapCOpipzaaa. In some embodiments of Formula (I), (I-1), (I-2), (I-3), (I-4), (I-5), (III-1), (III-2), (IV-1), or (IV-2). X2 is I, K, Cit, F4G, 4Py, 3Py, KCOpipzetOH, V, KCOpipzaa, Eva, Q, E, S, Ahp, F4COO, KCOpip4COO, MeQdMe, MeA, MeSMe, MeG, MeI, MeV, MeL, HseMe, MeY, Me3Py, MeHseMe, MeKAc, alI, TMe MeKCOpipzaa, MeQ, Hpr, MeTMe, or MeDapCOpipzaaa, each of which is optionally substituted. In some embodiments, X2 is optionally substituted 1. In some embodiments, X2 is optionally substituted K. In some embodiments, X2 is optionally substituted Cit. In some embodiments, X2 is optionally substituted F4G. In some embodiments, X2 is optionally substituted 4Py. In some embodiments, X2 is optionally substituted 3Py. In some embodiments, X2 is optionally substituted KCOpipzetOH. In some embodiments, X2 is optionally substituted V. In some embodiments, X2 is optionally substituted KCOpipzaa. In some embodiments, X2 is optionally substituted Eva. In some embodiments, X2 is optionally substituted Q. In some embodiments, X2 is optionally substituted E. In some embodiments, X2 is optionally substituted S. In some embodiments, X2 is optionally substituted MeL. In some embodiments, X2 is optionally substituted HseMe. In some embodiments, X2 is optionally substituted MeY. In some embodiments, X2 is optionally substituted Me3Py. In some embodiments, X2 is optionally substituted Ahp. In some embodiments, X2 is optionally substituted F4COO. In some embodiments, X2 is optionally substituted KCOpip4COO. In some embodiments, X2 is optionally substituted MeQdMe. In some embodiments, X2 is optionally substituted MeA. In some embodiments, X2 is optionally substituted MeSMe. In some embodiments, X2 is optionally substituted MeG. In some embodiments, X2 is optionally substituted MeI. In some embodiments, X2 is optionally substituted MeV. In some embodiments, X2 is optionally substituted MeHseMe. In some embodiments, X2 is optionally substituted MeKAc. In some embodiments, X2 is optionally substituted all. In some embodiments, X2 is optionally substituted TMe. In some embodiments, X2 is optionally substituted MeKCOpipzaa. In some embodiments, X2 is optionally substituted MeQ. In some embodiments, X2 is optionally substituted Hpr. In some embodiments, X2 is optionally substituted MeTMe. In some embodiments, X2 is optionally substituted. In some embodiments, X2 is optionally substituted MeDapCOpipzaaa.

In some embodiments of a peptide of Formula (I), (I-1), (I-2), (I-3), (I-4), (I-5), (III-1), (III-2), (IV-1), or (IV-2), X2 is any amino acid. In some embodiments, X2 is a D-amino acid. In some embodiments, X2 is an L-amino acid. In some embodiments, X2 is an N-alkylated amino acid. In some embodiments, X2 is an N-methylated amino acid. In some embodiments, X2 does not comprise a cyclic group. In some embodiments, X2 comprises a cyclic group. In some embodiments, X2 comprises a 5-6 membered heterocycloalkyl group. In some embodiments. X2 comprises a 5-6 membered heteroaryl group. In some embodiments, X2 comprises a phenyl group. In some embodiments, X2 is a peptoid.

In some embodiments of a peptide of Formula (I), (I-1), (I-2), (I-3), (I-4), (I-5), (III-1), (III-2), (IV-1), or (IV-2), X2 has a structure of

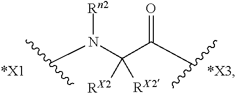

wherein,
   Rⁿ² is hydrogen or C₁₋₃ alkyl, wherein the alkyl is optionally substituted with one or more R^{X2a};
   R^{X2} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, - L^{X2}-heterocycloalkyl, -L^{X2} cycloalkyl, -L^{X2}-aryl, or -L^{X2}-heteroaryl, wherein each of the alkyl, heteroalkyl, alkenyl, alkynyl, aryl, heteroaryl, cycloalkyl, and heterocycloalkyl is optionally substituted with one or more R^{X2a};
   R^{X2′} is hydrogen or C₁₋₆alkyl, wherein the alkyl is optionally substituted with one or more R^{X2a}; or
   Rⁿ² and R^{X2′} are taken together with the intervening atoms to form a 5-to 6-membered heterocycloalkyl, which is optionally substituted with one or more R^{X2a};
   L^{X2} is C₁₋₆halkylene, C₁₋₆heteroalkylene, —O—, —S—, or —NR^{a}—, wherein the alkylene and heteroalkylene is optionally substituted with one or more R^{X2a};
   each R^{X2a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF^{a}, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{a}), —P(═O)R^{c}R^{d}, aryl, heteroaryl, cycloalkyl, heterocycloalkyl, ═O, ═S, or ═N(R^{c}), wherein each of the aryl, heteroaryl, cycloalkyl, heterocycloalkyl, alkyl, heteroalkyl, alkenyl, and alkynyl is optionally substituted with one or more R^{e};
   *X1 represents the point of attachment to X1;
   *X3 represents the point of attachment to X3; and
   other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (III-1), (III-2), (IV-1), or (IV-2), X2 has a structure of

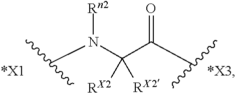

wherein:
   Rⁿ² is hydrogen or methyl;
   R^{X2} is hydrogen, C₁₋₆alkyl, C₁₋₆haloalkyl, C₁₋₆hydroxyalkyl, C₁₋₆aminoalkyl, C₁₋₆heteroalkyl, -L^{X2}-5-6 membered heterocycloalkyl, -L^{X2}-C₄₋₆cycloalkyl, -L^{X2}-C₆₋₁₀aryl, or -L^{X2}-5-10 membered heteroaryl, wherein each of the alkyl, heteroalkyl, aryl, heteroaryl, cycloalkyl, and heterocycloalkyl is optionally substituted with one or more R^{X2a};
   R^{X2′} is hydrogen or methyl; or
   Rⁿ² and R^{X2′} are taken together with the intervening atoms to form a 5-to 6-membered heterocycloalkyl, which is optionally substituted with one or more R^{X2a};
   L^{X2} is C₁₋₆alkylene or C₁₋₆heteroalkylene, wherein the alkylene and heteroalkylene is optionally substituted with one or more R^{X2a};
   each R^{X2a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NR^{a})R^{a}, —N═S(═O)R^{c}R^{d}, —NR^{a}S(═O)R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the alkyl, heteroalkyl, alkenyl, and alkynyl is optionally substituted with one or more R^{e};
   *X1 represents the point of attachment to X1;
   *X3 represents the point of attachment to X3; and other groups (such as R^{a}, R^{c}, R^{d}, and R^{e}) have the meanings defined in Formula (I).

In some embodiments, Rⁿ² is hydrogen. In some embodiments, Rⁿ² is C₁₋₃ alkyl. In some embodiments, Rⁿ² is methyl.

In some embodiments, R^{X2} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X2}-heterocycloalkyl, -L^{X2}-cycloalkyl, -L^{X2}-aryl, or -L^{X2}-heteroaryl, wherein each of the alkyl, alkenyl, alkynyl, heteroalkyl, aryl, heteroaryl, cycloalkyl, and heterocycloalkyl is optionally substituted with one or more R^{X2a}.

In some embodiments, R^{X2} is hydrogen, C₁₋₆alkyl, C₁₋₆haloalkyl, C₁₋₆hydroxyalkyl, C₁₋₆aminoalkyl, C₁₋₆heteroalkyl. -L^{X2}-5-6 membered heterocycloalkyl, -L^{X2}-C₄₋₆cycloalkyl, -L^{X2}-C₆₋₁₀aryl, or -L^{X2}-5-10 membered heteroaryl, wherein each of the alkyl, heteroalkyl, aryl, heteroaryl, cycloalkyl, and heterocycloalkyl is optionally substituted with one or more R^{X2a}. In some embodiments, R^{X2} is hydrogen, C₁₋₆alkyl, C₁₋₆heteroalkyl, -L^{X2}-piperidinyl, L^{X2}-piperazinyl, -L^{X2}-phenyl, or -L^{X2}-pyridinyl, wherein each of the alkyl, heteroalkyl, phenyl, pyridinyl, piperidinyl, and piperazinyl is optionally substituted with one or more R^{X2a}.

In some embodiments, R^{X2} is hydrogen. In some embodiments, R^{X2} is optionally substituted C₁₋₆alkyl. In some embodiments, R^{X2} is C₁₋₆alkyl optionally substituted with one or more R^{X2a}. In some embodiments, R^{X2} is optionally substituted C₁₋₆heteroalkyl. In some embodiments, R^{X2} is C₁₋₆heteroalkyl optionally substituted with one or more R^{X2a}. In some embodiments, R^{X2} is C₁₋₆alkyl, C₁₋₆haloalkyl, C₁₋₆hydroxyalkyl, C₁₋₆aminoalkyl, or C₁₋₆heteroalkyl, wherein each of which is optionally substituted with one or more R^{X2a}. In some embodiments, R^{X2} is C₂₋₆alkenyl or C₂₋₆alkynyl, wherein each of which is optionally substituted with one or more R^{X2a}. In some embodiments, R^{X2} is aryl, heteroaryl, cycloalkyl, or heterocycloalkyl, wherein each of which is optionally substituted with one or more R^{X2a}. In some embodiments, R^{X2} is aryl or heteroaryl, wherein each of which is optionally substituted with one or more R^{X2a}. In some embodiments, R^{X2} is cycloalkyl or heterocycloalkyl, wherein each of which is optionally substituted with one or more R^{X2a}. In some embodiments, R^{X2} is -L^{X2}-heterocycloalkyl, -L^{X2}-cycloalkyl, -L^{X2}-aryl, or -L^{X2}-heteroaryl, wherein each of which is optionally substituted with one or more R^{X2a}. In some embodiments, R^{X2} is -L^{X2}-heterocycloalkyl, which is optionally substituted with one or more R^{X2a}. In some embodiments, R^{X2} is -L^{X2}-cycloalkyl, which is optionally substituted with one or more R^{X2a}. In some embodiments, R^{X2} is -L^{X2}-aryl, which is optionally substituted with one or more R^{X2a}. In some embodiments, R^{X2} is -L^{X2}-heteroaryl, which is optionally substituted with one or more R^{X2a}.

In some embodiments, R^{X2′} is hydrogen. In some embodiments, R^{X2′} is C₁₋₆alkyl, wherein the alkyl is optionally substituted with one or more R^{X2a}. In some embodiments, R^{X2′} is C₁₋₆alkyl. In some embodiments, R^{X2′} is methyl.

In some embodiments, Rⁿ² and R^{X2′} are taken together with the intervening atoms to form a 5-to 6-membered heterocycloalkyl, which is optionally substituted with one or more R^{X2a}.

In some embodiments, L^{X2} is C₁₋₆alkylene, wherein the alkylene is optionally substituted with one or more R^{X2a}. In some embodiments, L^{X2} is C₁₋₆alkylene. In some embodiments, L^{X2} is C₁₋₆heteroalkylene, wherein the heteroalkylene is optionally substituted with one or more R^{X2}. In some embodiments, L^{X2} is C₁₋₆heteroalkylene. In some embodiments, L^{X2} is —O—. In some embodiments, L^{X2} is —S—. In some embodiments, L^{X2} is —NR^{a}—. In some embodiments, L^{X2} is —NH—.

In some embodiments, each R^{X2a} is independently halogen, C₁₋₆ alkyl, C₁-C₆haloalkyl, C₁-C₆hydroxyalkyl, C₁-C₆aminoalkyl, C₁-C₆heteroalkyl, cycloalkyl, heterocycloalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)R^{a}, —NR^{a}C(═NH)(NR²), —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X2a} is independently halogen, C₁₋₆ alkyl, C₁-C₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR)₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R_{d}, or ═O, wherein each of the alkyl and heteroalkyl is optionally substituted with one or more R^{c}.

In some embodiments, each R^{X2a} is independently C₁₋₆alkyl, C₁₋₆ alkoxyl, C₁-C₆haloalkyl, C₁-C₆hydroxyalkyl, C₁-C₆aminoalkyl, OH, —CN, —NO₂, —NH₂, —C(═O)R^{a}, —C(═O)OR^{a}, —NR^{a}C(═O)NR^{c}R^{d}, or oxo.

In some embodiments, Rⁿ² is methyl, R^{X2′} is hydrogen, and R^{X2} is C₁₋₆alkyl. In some embodiments, Rⁿ² is methyl, R^{X2′} is hydrogen, and R^{X2} is branched C₁₋₆alkyl. In some embodiments, Rⁿ² is methyl, R^{X2′} is hydrogen, and R^{X2} is branched C₃₋₆alkyl. In some embodiments, R^{X2} is —CH(CH₂)CH₂CH₃.

In some embodiments of Formula (I), (I-1), (I-2), (I-3), (I-4), (I-5), (II-1), (II-2), (IV-1), or (IV-2), X3 is a polar and/or an L-amino acid. In some embodiments, X3 is an amino acid comprising a hydrophilic side chain (e.g., D, K, Q, or a variant thereof) or an N-methylated variant thereof. In some embodiments, X3 is absent.

In some embodiments of Formula (I), (I-1), (I-2), (I-3), (I-4), (I-5), (II-1), (III-2), (IV-1), or (IV-2), X3 is D, Har, KCOpipzetOH, Cit, KCOmeglumine, KCOpipzaa, A4paa, Q, A, E, MeD, S, N, Hgl, F4COO, KCOpip4COO, KAc, Hgn, MeY, or DapCOpipzaa. In some embodiments of Formula (I), (I-1), (I-2), (I-3), (I-4), (I-5), (IV-1), (III-2), (IV-1), or (IV-2). X3 is D, Har, KCOpipzetOH, Cit, KCOmeglumine, KCOpipzaa, A4paa, Q. A, E. MeD, S. N, Hgl, F4COO, KCOpip4COO, KAc, Hgn, MeY, or DapCOpipzaa, each of which is optionally substituted. In some embodiments, X3 is optionally substituted D. In some embodiments, X3 is optionally substituted Har. In some embodiments, X3 is optionally substituted KCOpipzetOH. In some embodiments, X3 is optionally substituted Cit. In some embodiments, X3 is optionally substituted KCOmeglumine. In some embodiments, X3 is optionally substituted KCOpipzaa. In some embodiments, X3 is optionally substituted A4paa. In some embodiments, X3 is optionally substituted Q. In some embodiments, X3 is optionally substituted A. In some embodiments, X3 is optionally substituted E. In some embodiments. X3 is optionally substituted MeD. In some embodiments, X3 is optionally substituted S. In some embodiments, X3 is optionally substituted N. In some embodiments, X3 is optionally substituted Hgl. In some embodiments, X3 is optionally substituted F4COO. In some embodiments, X3 is optionally substituted KCOpip4COO. In some embodiments, X3 is optionally substituted KAc. In some embodiments, X3 is optionally substituted Ign. In some embodiments. X3 is optionally substituted MeY. In some embodiments, X3 is optionally substituted DapCOpipzaa.

In some embodiments of a peptide of Formula (I), (I-1), (I-2), (I-3), (I-4), (I-5), (III-1), (III-2), (IV-1), or (IV-2), X3 is any amino acid. In some embodiments, X3 is a D-amino acid. In some embodiments. X3 is an L-amino acid. In some embodiments, X3 is an N-alkylated amino acid. In some embodiments, X3 is an N-methylated amino acid. In some embodiments, X3 does not comprise a cyclic group. In some embodiments, X3 comprises a cyclic group. In some embodiments. X3 comprises a 5-6 membered heterocycloalkyl group. In some embodiments, X3 comprises a 5-6 membered heteroaryl group. In some embodiments, X3 comprises a phenyl group. In some embodiments, X3 is a peptoid. In some embodiments, X3 is a polar amino acid.

In some embodiments of a peptide of Formula (I), (I-1), (I-2), (I-3), (I-4), (I-5), (III-1), (III-2), (IV-1), or (IV-2), X3 has a structure of

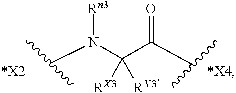

wherein:
Rⁿ³ is hydrogen or C₁₋₃alkyl, wherein the alkyl is optionally substituted with one or more R^{X3a};
R^{X3} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X3}-heterocycloalkyl, -L^{X3}-cycloalkyl, -L^{X3}-aryl, or -L^{X3}-heteroaryl, wherein each of the alkyl, heteroalkyl, alkenyl, alkynyl, aryl, heteroaryl, cycloalkyl, and heterocycloalkyl is optionally substituted with one or more R^{X3a};
R^{X3′} is hydrogen or C₁₋₆alkyl, wherein the alkyl is optionally substituted with one or more R^{X3a}; or
Rⁿ³ and R^{X3′} are taken together with the intervening atoms to form a 5-to 6-membered heterocycloalkyl, which is optionally substituted with one or more R^{X3a};
L^{X3} is C₁₋₆alkylene, C₁₋₆heteroalkylene, —O—, —S—, or —NR^{a}—, wherein the alkylene and heteroalkylene is optionally substituted with one or more R^{X3a};
each R^{X3a} is independently halogen, C₁₋₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R², —SF₅, —S(═O)₂NR^{c}R^{d}, —S(═O)(═NR)R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR⁹, —OC(═O)NR^{c}R^{d}, —NR^{a}C(═O)R^{a}, —NR^{a}C(═O)OR³, —NR^{a}C(═O)NR_{c}R^{d}, —C(═O)NR^{c}R^{d}, —P(═O)(OR^{c})(OR^{d}), —P(═O)R^{c}R^{a}, aryl, heteroaryl, cycloalkyl heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
*X2 represents the point of attachment to X2;
*X4 represents the point of attachment to X4;
other groups (such as R^{a}, R^{c}, R^{d} and R^{e}) have the meanings defined in Formula (I).

In some embodiments of a peptide of Formula (I), (I-1), (I-2), (I-3), (I-4), (I-5), (II-1), (III-2), (IV-1), or (IV-2), X3 has a structure of

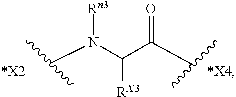

wherein:
Rⁿ³ is hydrogen or methyl;
R^{X3} is C₁₋₆alkyl, C₁₋₆haloalkyl, C₁₋₆hydroxyalkyl, C₁₋₆aminoalkyl, C₁₋₆heteroalkyl, -L^{X3}-5-6 membered heterocycloalkyl, -L^{X3}-C₃₋₆cycloalkyl, -L^{X3}C₆₋₁₀aryl, or -L^{X3}-5-10 membered heteroaryl, wherein each of the alkyl, heteroalkyl, aryl, heteroaryl, cycloalkyl, and heterocycloalkyl is optionally substituted with one or more R^{X3a};
L^{X3} is C₁₋₆alkylene or C₁₋₆heteroalkylene, wherein the alkylene and heteroalkylene is optionally substituted with one or more R^{X3a};
each R^{X3a} is independently halogen, C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR³, —NR^{c}R^{d}, —S(═O)R^{a}, —S(═O)₂R^{a}, —SF₅, —S(═O)₂NR^{c}R_{d}, —S(═O)(═NR)R^{a}, —N═S(═O)R^{c}R^{d}, —NR^{a}S(═O)₂R^{a}, amidinyl, —NR^{a}C(═NH)(NR^{a})₂, —NR^{c}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR²C(═O)OR², —NR^{a}C(═O)NR^{c}R^{d}, —C(═O)NR^{c}R^{d}, —P(═O)(OR^{c})(OR^{c}), —P(═O)R^{c}R^{d}, aryl, heteroaryl, cycloalkyl, heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
*X2 represents the point of attachment to X2;
*X4 represents the point of attachment to X4; and
other groups (such as R^{a}, R^{c}, R⁴, and R^{c}) have the meanings defined in Formula (I).

In some embodiments, Rⁿ³ is hydrogen. In some embodiments, Rⁿ³ is C₁₋₃alkyl. In some embodiments, Rⁿ³ is methyl.

In some embodiments, R^{X3} is hydrogen, C₁₋₆alkyl, C₁₋₆haloalkyl, C₁₋₆hydroxyalkyl, C₁₋₆ aminoalkyl, C₁₋₆heteroalkyl, C₂₋₆alkenyl, C₂₋₆alkynyl, aryl, heteroaryl, cycloalkyl, heterocycloalkyl, -L^{X3}-heterocycloalkyl, -L^{X3}-cycloalkyl, -L^{X3}-aryl, or -L^{X3}-heteroaryl, wherein each of the alkyl, alkenyl, alkynyl, heteroalkyl, aryl, heteroaryl, cycloalkyl, and heterocycloalkyl is optionally substituted with one or more R^{X3a}. In some embodiments, R^{X3} is C₁₋₆alkyl, C₁₋₆haloalkyl, C₁₋₆hydroxyalkyl, C₁₋₆aminoalkyl, C₁₋₆heteroalkyl, -L^{X3}-5-6 membered heterocycloalkyl, -L^{X3}-C₃₋₆cycloalkyl, -L^{X3}-C₆₋₁₀aryl, or -L^{X3}-5-10 membered heteroaryl, wherein each of the alkyl, heteroalkyl, aryl, heteroaryl, cycloalkyl, and heterocycloalkyl is optionally substituted with one or more R^{X3a}.

In some embodiments, R^{X3} is hydrogen, C₁₋₆alkyl, C₁₋₆haloalkyl, C₁₋₆hydroxyalkyl, C₁₋₆aminoalkyl, C₁₋₆heteroalkyl, -L^{X3}-5-6 membered heterocycloalkyl, -L^{X3}-C₄₋₆cycloalkyl, -L^{X3}-C₆₋₁₀aryl, or -L^{X3}-5-10 membered heteroaryl, wherein each of the alkyl, heteroalkyl, aryl, heteroaryl, cycloalkyl, and heterocycloalkyl is optionally substituted with one or more R^{X3a}. In some embodiments. R^{X3} is hydrogen. C₁₋₆alkyl, C₁₋₆heteroalkyl, -L^{X3}-piperidinyl, L^{X3}-piperazinyl, -L^{X3}-phenyl, or -L^{X3}-pyridinyl, wherein each of the alkyl, heteroalkyl, phenyl, pyridinyl, piperidinyl, and piperazinyl is optionally substituted with one or more R^{X3a}.

In some embodiments, R^{X3} is hydrogen. In some embodiments, R^{X3} is optionally substituted C₁₋₆alkyl. In some embodiments, R^{X3} is C₁₋₆alkyl optionally substituted with one or more R^{X3}. In some embodiments, R^{X3} is optionally substituted C₁₋₆heteroalkyl. In some embodiments, R^{X3} is C₁₋₆heteroalkyl optionally substituted with one or more R^{X3a}. In some embodiments, R^{X3} is C₁₋₆alkyl, C₁-C_{6aloalkyl}, C₁₋₆hydroxyalkyl, C₁₋₆aminoalkyl, or C₁₋₆heteroalkyl, wherein each of which is optionally substituted with one or more R^{X3a}. In some embodiments, R^{X3} is C₂₋₆alkenyl or C₂₋₆alkynyl, wherein each of which is optionally substituted with one or more R³. In some embodiments, R^{X3} is aryl, heteroaryl, cycloalkyl, or heterocycloalkyl, wherein each of which is optionally substituted with one or more R-J. In some embodiments, R^{X3} is aryl or heteroaryl, wherein each of which is optionally substituted with one or more R^{X3a}. In some embodiments, R^{X3} is cycloalkyl or heterocycloalkyl, wherein each of which is optionally substituted with one or more R^{X3a}. In some embodiments, R^{X3} is -L^{X3}-heterocycloalkyl, -L^{X3}-cycloalkyl, -L^{X3}-aryl, or -L^{X3}-heteroaryl, wherein each of which is optionally substituted with one or more R^{X3a}. In some embodiments, R^{X3} is -L^{X3}-heterocycloalkyl, which is optionally substituted with one or more R^{X3a}. In some embodiments, R^{X3} is -L³-cycloalkyl, which is optionally substituted with one or more R^{X3a}. In some embodiments, R^{X3} is -L^{X3}-aryl, which is optionally substituted with one or more R^{X3a}. In some embodiments, R^{X3} is -L^{X3}-heteroaryl, which is optionally substituted with one or more R^{X3a}.

In some embodiments, R^{X3} is hydrogen. In some embodiments, R^{X3′} is C₁₋₆alkyl, wherein the alkyl is optionally substituted with one or more R^{X3a}. In some embodiments, R^{X3} is C₁₋₆alkyl. In some embodiments, R^{X3′} is methyl.

In some embodiments, Rⁿ³ and R^{X3′} are taken together with the intervening atoms to form a 5-to 6-membered heterocycloalkyl, which is optionally substituted with one or more R^{X3a}.

In some embodiments, L^{X3} is C₁₋₆alkylene, wherein the alkylene is optionally substituted with one or more R^{X3a}. In some embodiments, L^{X3} is C₁₋₆alkylene. In some embodiments, L^{X3} is C₁₋₆heteroalkylene, wherein the heteroalkylene is optionally substituted with one or more R^{X3a}. In some embodiments, L^{X3} is C₁₋₆heteroalkylene. In some embodiments, L^{X3} is —O—. In some embodiments, L^{X3} is —S—. In some embodiments, L^{X3} is —NR^{a}—. In some embodiments, L^{X3} is —NH—.

In some embodiments, each R^{X3a} is independently halogen, C₁₋₆ alkyl, C₁-C₆haloalkyl, C₁-C₆hydroxyalkyl, C₁-C₆aminoalkyl, C₁-C₆heteroalkyl, cycloalkyl, heterocycloalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)R^{a}, —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X3a} is independently halogen, C₁₋₆ alkyl, C₁-C₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X3a} is independently C₁₋₆alkyl, C₁₋₆alkoxyl, C₁-C₆haloalkyl, C₁-C₆hydroxyalkyl, C₁-C₆aminoalkyl, OH, —CN, —NO₂, —NH₂, —C(═O)R^{a}, —C(═O)OR^{a}, —NR^{a}C(═O)NR^{c}R^{d}, or oxo.

In some embodiments, Rⁿ³ is hydrogen, R^{X3′} is hydrogen, and R^{X3} is C₁₋₆alkyl optionally substituted with one or more R^{X3a}. In some embodiments, Rⁿ³ is hydrogen. R^{X3′} is hydrogen, and R^{X3} is C₁₋₃alkyl optionally substituted with one or more R^{X3a}. In some embodiments, Rⁿ³ is hydrogen, R^{X3′} is hydrogen, and R^{X3} is C₁₋₃alkyl substituted with —COOH.

In some embodiments of Formula (I), (I-1), (I-2), (I-3), (I-4), (I-5), (III-1), (III-2), (IV-1), or (IV-2), X4 is a polar and/or an L-amino acid. In some embodiments, X4 is an N-methylated amino acid, a polar amino acid (e.g., D, K, Q, S. or a variant thereof), or peptoid (e.g. EtG, MeeG, CmG. CmpG CrmG. CeG, or CrpG).

In some embodiments of Formula (I), (I-1), (I-2), (I-3), (I-4), (I-5), (III-1), (III-2), (IV-1), or (IV-2), X4 is D, Har, KCOpipzetOH, KCOmeglumine, KCOpipzaa. A4paa, Q, A, E, MeD, S, N, Hgl, F4COO, KCOpip4COO, dd, MeQ, MeQdMe, MeA, MeSMe, MeG, EtG, MeecG, CmG, CmpG, CrmG, CecG, CrpG, MeK, MeKAc, MeHgl, Hgn, MeDapCOpipzaa, MeKCOpipzaa, Medd, Cit, MeCit, MeN, MeS, MeF, MeY, W5N, or MeA4paa. In some embodiments of Formula (I), (I-1), (I-2), (I-3), (I-4), (I-5), (II-1), (III-2), (IV-1), or (IV-2), X4 is D, liar, KCOpipzetOH, KCOmeglumine, KCOpipzaa, A4paa, Q, A, E, MeD, S, N, Hgl, F4COO, KCOpip4COO, dd, MeQ, MeQdMe, MeA, MeSMe, MeG, EtG, MeeG, CmG, CmpG, CrmG, CeG, CrpG, MeK, MeKAc, MeHgl, Hgn, MeDapCOpipzaa, MeKCOpipzaa, Medd, Cit, MeCit, MeN, MeS, MeE, MeY, W5N, or MeA4paa, each of which is optionally substituted. In some embodiments, X4 is optionally substituted D. In some embodiments, X4 is optionally substituted Har. In some embodiments, X4 is optionally substituted KCOpipzetOH. In some embodiments. X4 is optionally substituted KCOmeglumine. In some embodiments. X4 is optionally substituted KCOpipzaa. In some embodiments, X4 is optionally substituted A4paa. In some embodiments, X4 is optionally substituted Q. In some embodiments, X4 is optionally substituted A. In some embodiments, X4 is optionally substituted E. In some embodiments. X4 is optionally substituted MeD. In some embodiments, X4 is optionally substituted S. In some embodiments, X4 is optionally substituted N. In some embodiments, X4 is optionally substituted Hgl. In some embodiments, X4 is optionally substituted F4COO. In some embodiments, X4 is optionally substituted KCOpip4COO. In some embodiments, X4 is optionally substituted dd. In some embodiments, X4 is optionally substituted MeQ. In some embodiments, X4 is optionally substituted MeQdMe. In some embodiments, X4 is optionally substituted MeA. In some embodiments, X4 is optionally substituted MeSMe. In some embodiments, X4 is optionally substituted MeG. In some embodiments, X4 is optionally substituted EtG. In some embodiments, X4 is optionally substituted MeeG. In some embodiments, X4 is optionally substituted CmG. In some embodiments, X4 is optionally substituted CmpG. In some embodiments, X4 is optionally substituted CrmG. In some embodiments, X4 is optionally substituted CeG. In some embodiments, X4 is optionally substituted CrpG. In some embodiments. X4 is optionally substituted MeK. In some embodiments, X4 is optionally substituted MeKAc. In some embodiments, X4 is optionally substituted MeHgl. In some embodiments. X4 is optionally substituted Ign. In some embodiments, X4 is optionally substituted MeDapCOpipzaa. In some embodiments, X4 is optionally substituted MeKCOpipzaa. In some embodiments, X4 is optionally substituted Medd. In some embodiments, X4 is optionally substituted Cit. In some embodiments, X4 is optionally substituted MeCit. In some embodiments, X4 is optionally substituted MeN. In some embodiments, X4 is optionally substituted MeS. In some embodiments, X4 is optionally substituted MeE. In some embodiments, X4 is optionally substituted MeY. In some embodiments, X4 is optionally substituted W5N. In some embodiments, X4 is optionally substituted MeA4paa.

In some embodiments of a peptide of Formula (I), (I-1), (I-2), (I-3), (I-4), (I-5), (III-1), (III-2), (IV-1), or (IV-2), X4 is any amino acid. In some embodiments, X4 is a D-amino acid. In some embodiments, X4 is an L-amino acid. In some embodiments, X4 is an N-alkylated amino acid. In some embodiments, X4 is au N-methylated amino acid. In some embodiments. X4 does not comprise a cyclic group. In some embodiments, X4 comprises a cyclic group. In some embodiments, X4 comprises a 5-6 membered heterocycloalkyl group. In some embodiments, X4 comprises a 5-6 membered heteroaryl group. In some embodiments, X4 comprises a phenyl group. In some embodiments, X4 is a peptoid. In some embodiments, X4 is a polar amino acid.

In some embodiments of a peptide of Formula (I), (I-1), (I-2), (I-3), (I-4), (I-5), (III-1), (III-2), (IV-1), or (IV-2), X4 has a structure of

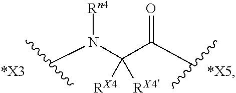

wherein:
R⁴ is hydrogen or C₁₋₃ alkyl, wherein the alkyl is optionally substituted with one or more R^{X4a};
R^{X4} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X4}-heterocycloalkyl, -L^{X4}-cycloalkyl, -L^{X4}-aryl, or -L^{X4}-heteroaryl, wherein each of the alkyl, heteroalkyl, alkenyl, alkynyl, aryl, heteroaryl, cycloalkyl, and heterocycloalkyl is optionally substituted with one or more R^{X4a};
R^{X4′} is hydrogen or C₁₋₆alkyl, wherein the alkyl is optionally substituted with one or more R^{X4a}; or
Rⁿ⁴ and R^{X4′} are taken together with the intervening atoms to form a 5-to 6-membered heterocycloalkyl, which is optionally substituted with one or more R^{X4′};
L^{X4} is C₁₋₆alkylene or C₁₋₆heteroalkylene, —O—, —S—, or —NR^{a}—, wherein the alkylene and heteroalkylene is optionally substituted with one or more R^{X4a};
each R^{X4a} is independently halogen. C₁₋₆ alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR^{a}, —SR^{a}, —NR^{c}R^{d}, —S(═O)R^{a}, —S(═O)₂R^{a}, —SF₅, —S(═O)₂NR^{c}R^{d}, —S(═O)(═NRₐ)Rₐ, —N═S(═O)R^{c}R^{d}, —NR^{a}S(═O)₂R^{a}, amidinyl, —NR^{a}C(═NH)(NR², —NR^{a}S(═O)₂NR_{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}. —NR²C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
*X3 represents the point of attachment to X3;
*X5 represents the point of attachment to X5; and other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (III-1), (III-2), (IV-1), or (IV-2), X4 has a structure of

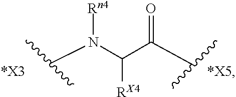

wherein:
Rⁿ⁴ is hydrogen or C₁₋₃ alkyl, wherein the alkyl is optionally substituted with one or more R^{X4a};
R^{X4} is hydrogen, C₁₋₆alkyl, C₁₋₆haloalkyl, C₁₋₆hydroxyalkyl, C₁₋₆aminoalkyl, C₁₋₆heteroalkyl, -L^{X4}-5-6 membered heterocycloalkyl, -L^{X4}-C₃cycloalkyl, -L^{X4}-C₆₋₁₀aryl, or -L^{X4}-5-10 membered heteroaryl, wherein each of the alkyl, heteroalkyl, aryl, heteroaryl, cycloalkyl, and heterocycloalkyl is optionally substituted with one or more R^{X4a};
L^{X4} is C₁₋₆alkylene or C₁₋₆heteroalkylene, wherein the alkylene and heteroalkylene is optionally substituted with one or more R^{X4a};
each R^{X4}, is independently halogen, C₁₋₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, —CN, —NO₂, —OR², —SR^{a}, —NR^{c}R^{d}, —S(═O)R^{a}, —S(═O)₂R², —SF₅, —S(═O)₂NR^{c}R^{d}, —S(═O)(═NR^{a})R^{a}, —N═S(═O)R^{c}R^{d}, —NR^{a}S(═O)₂R^{a}, amidinyl, —NR^{a}C(═NH)(NR^{a})₂, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR), —P(═O)R^{c}R^{a}, aryl, heteroaryl, cycloalkyl, heterocycloalkyl, ═O, ═S, or ═N(R^{a}), wherein each of the aryl, heteroaryl, cycloalkyl, heterocycloalkyl, alkyl, heteroalkyl, alkenyl, and alkynyl is optionally substituted with one or more R^{e};
*X3 represents the point of attachment to X3;
*X5 represents the point of attachment to X5; and
other groups (such as R^{a}, R^{c}, R^{d}, and R^{e}) have the meanings defined in Formula (I).

In some embodiments, Rⁿ⁴ is hydrogen. In some embodiments, Rⁿ⁴ is C₁₋₃ alkyl. In some embodiments, Rⁿ⁴ is methyl.

In some embodiments, R^{X4} is hydrogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X4}-heterocycloalkyl, -L^{X4}-cycloalkyl, -L^{X4}-aryl, or -L^{X4}-heteroaryl, wherein each of the alkyl, alkenyl, alkynyl, heteroalkyl, aryl, heteroaryl, cycloalkyl, and heterocycloalkyl is optionally substituted with one or more R^{X4a}. In some embodiments, R^{X4} is C₁₋₆alkyl, C₁₋₆haloalkyl, C₁₋₆hydroxyalkyl, C₁₋₆aminoalkyl, C₁₋₆heteroalkyl, -L^{X4}-5-6 membered heterocycloalkyl, -L^{X4}-C₃₋₆cycloalkyl, -L^{X4}-C₆₋₁₀aryl, or -L^{X4}-5-10 membered heteroaryl, wherein each of the alkyl, heteroalkyl, aryl, heteroaryl, cycloalkyl, and heterocycloalkyl is optionally substituted with one or more R^{X4a}.

In some embodiments, R^{X4} is hydrogen, C₁₋₆alkyl, C₁₋₆haloalkyl, C₁₋₆hydroxyalkyl, C₁₋₆aminoalkyl, C₁₋₆heteroalkyl, -L^{X4}-5-6 membered heterocycloalkyl, -L^{X4}-C₄₋₆cycloalkyl, -L^{X4}-C₆₋₁₀aryl, or -L^{X4}-5-10 membered heteroaryl, wherein each of the alkyl, heteroalkyl, aryl, heteroaryl, cycloalkyl, and heterocycloalkyl is optionally substituted with one or more R^{X4a}. In some embodiments, R^{X4} is hydrogen. C₁₋₆alkyl, C₁₋₆heteroalkyl, -L^{X4}-piperidinyl, L^{X4}-piperazinyl, -L^{X4}-phenyl, or -L^{X4}-pyridinyl, wherein each of the alkyl, heteroalkyl, phenyl, pyridinyl, piperidinyl, and piperazinyl is optionally substituted with one or more R^{X4a}.

In some embodiments, R^{X4} is hydrogen. In some embodiments, R^{X4} is optionally substituted C₁₋₆alkyl. In some embodiments, R^{X4} is C₁₋₆alkyl optionally substituted with one or more R^{X4a}. In some embodiments, R^{X4} is optionally substituted C₁₋₆heteroalkyl. In some embodiments, R^{X4} is C₁₋₆heteroalkyl optionally substituted with one or more R^{X4a}. In some embodiments, R^{X4} is C₁₋₆alkyl, C₁₋₆haloalkyl, C₁₋₆hydroxyalkyl, C₁₋₆aminoalkyl, or C₁₋₆heteroalkyl, wherein each of which is optionally substituted with one or more R^{X4a}. In some embodiments, R^{X4} is C₂₋₆alkenyl or C₂₋₆alkynyl, wherein each of which is optionally substituted with one or more R^{X4a}. In some embodiments, R^{X4} is aryl, heteroaryl, cycloalkyl, or heterocycloalkyl, wherein each of which is optionally substituted with one or more R^{X4a}. In some embodiments, R^{X4} is aryl or heteroaryl, wherein each of which is optionally substituted with one or more R^{X4a}. In some embodiments, R^{X4} is cycloalkyl or heterocycloalkyl, wherein each of which is optionally substituted with one or more R^{X4a}. In some embodiments, R^{X4} is -L^{X4}-heterocycloalkyl, -L^{X4}-cycloalkyl, -L^{X4}-aryl, or -L^{X4}-heteroaryl, wherein each of which is optionally substituted with one or more R^{X4a}. In some embodiments, R^{X4} is -L^{X4}-heterocycloalkyl, which is optionally substituted with one or more R^{X4a}. In some embodiments, R^{X4} is -L^{X4}-cycloalkyl, which is optionally substituted with one or more R^{X4a}. In some embodiments, R^{X4} is -L^{X4}-aryl, which is optionally substituted with one or more R^{X4a}. In some embodiments, R^{X4} is -L^{X4}-heteroaryl, which is optionally substituted with one or more R^{X4a}.

In some embodiments, R^{X4′} is hydrogen. In some embodiments, R^{X4′} is C₁₋₆alkyl, wherein the alkyl is optionally substituted with one or more R^{X4a}. In some embodiments, R^{X4′} is C₁₋₆alkyl. In some embodiments, R^{X4′} is methyl.

In some embodiments, Rⁿ⁴ and R^{X4′} are taken together with the intervening atoms to form a 5-to 6-membered heterocycloalkyl, which is optionally substituted with one or more R^{X42}.

In some embodiments, L^{X4} is C₁₋₆alkylene, wherein the alkylene is optionally substituted with one or more R^{X4a}. In some embodiments, L^{X4} is C₁₋₆alkylene. In some embodiments, L^{X4} is C₁₋₆heteroalkylene, wherein the heteroalkylene is optionally substituted with one or more R^{X4a}. In some embodiments, L^{X4} is C₁₋₆heteroalkylene. In some embodiments, L^{X4} is —O—. In some embodiments, L^{X4} is —S—. In some embodiments, LNA is —NR^{a}—. In some embodiments, L^{X4} is —NH—.

In some embodiments, each R^{X4a} is independently halogen, C₁₋₆ alkyl, C₁-C₆haloalkyl, C₁-C₆hydroxyalkyl, C₁-C₆aminoalkyl, C₁-C₆heteroalkyl, cycloalkyl, heterocycloalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR)₂, —C(═O)R^{a}, —C(═O)OR^{a}, —OC(═O)R^{a}, —OC(═O)OR², —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X4a} is independently halogen, C₁₋₆ alkyl, C₁-C₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR^{a})₂, —C(═O)R^{a}, —C(═O)OR², —OC(═O)R², —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, each R^{X4a} is independently C₁₋₆ alkyl, C₁₋₆alkoxyl, C₁-C₆haloalkyl, C₁-C₆hydroxyalkyl, C₁-C₆aminoalkyl, OH, —CN, —NO₂, —NH₂, —C(═O)R^{a}, —C(═O)OR^{a}, —NR^{a}C(═O)NR^{c}R^{d}, or oxo. In some embodiments, each R^{X4a} is independently halogen, C₁₋₆ alkyl, C₁-C₆haloalkyl, C₁-C₆hydroxyalkyl, C₁-C₆aminoalkyl, —OR^{a}, —CN, —NO₂, —NH₂, —C(═O)R², —C(═O)OR^{a}, —NR^{a}C(═O)NR^{c}R^{d}, or oxo.

In some embodiments, Rⁿ⁴ is methyl, R^{X4′} is hydrogen, and R^{X4} is C₁₋₆alkyl optionally substituted with one or more R^{X4a}. In some embodiments, Rⁿ⁴ is methyl. R^{X4′} is hydrogen, and R^{X4} is C₁₋₃alkyl optionally substituted with one or more R^{X4a}. In some embodiments, Rⁿ⁴ is methyl, R^{X4′} is hydrogen, and R^{X4} is C₁₋₆alkyl optionally substituted with one or more —C(═O)NH₂. In some embodiments, Rⁿ⁴ is methyl, R^{X4′} is hydrogen, and R^{X4} is C₁₋₃alkyl substituted with —C(═O)NH₂.

In some embodiments of Formula (I), (I-1), (I-2), (I-3), (I-4), (I-5), (III-1), (III-2), (IV-1), or (IV-2), X5 has an aromatic side chain, such as F, Y, or a variant thereof. In some embodiments, X5 is F, or a variant thereof comprising a phenyl, pyridinyl, or naphthalyl, wherein said phenyl, pyridinyl, or naphthalyl is optionally substituted with one or more substituents each independently selected from halogen, —C₁₋₃alkyl. —OH, —NH₂, —CN, —C(═O)OH, —C(═O)NH₂, —NHC(═O)CH₃, —C₁₋₃alkylene-C(═O)OH, —C₁₋₃alkylene-C(═O)NH₂, —O—C₁₋₃alkylene-C(═O)OH, —O—C₁₋₃alkylene-C(═O)NH₂, —C₁₋₃alkylene-C(═O)-5-to 6-membered heterocycloalkylene-C₁₋₃alkylene-C(═O)OH, —O—C₁₋₃alkylene-C(═O)-5-to 6-membered heterocycloalkylene-C₁₋₃alkylene-C(═O)OH, —C₁₋₃alkylene-NHC(═O)-5-to 6-membered heterocycloalkylene-C₁₋₃alkylene-C(═O)OH, —O—C₁₋₃alkylene-NHC(═O)-5-to 6-membered heterocycloalkylene-C₁₋₃alkylene-C(═O)OH, and —NH—C₁₋₃alkylene-C(═O)OH. In some embodiments, X5 is Y, or a variant thereof comprising a hydroxyphenyl, wherein the hydrogen atom in hydroxyphenyl of Y or of the variant is optionally substituted with one or more substituents selected from —C₁₋₃alkyl, and —C₁₋₃ alkylene-C(═O)OH.

In some embodiments of Formula (I), (I-1), (I-2), (I-3), (I-4), (I-5), (II-1), (III-2), (IV-1), or (IV-2), X5 is Y, F3G, 3Py6COO,4Py2NH2, 3Py5COO, F3COO, 3Py6NHAc, F, F4C, F40Me, F4COO, Nal2, F3aao, F4aa, F4aao, 3Py6NHaa, 5Pdo, F3CON. F4F, F40Et, F4Me, F4CON, F4CONPEG4Me, F30Me. F3CON, YaeCOpipzaa, F4aaopipzaa, 4Pdo, 3Py6CON, Atp, Cha4cH, Cha4tH, Cha4cOMe, A1mor, or F4amCOpipzaa. In some embodiments of Formula (I), (I-1), (I-2), (I-3), (I-4), (I-5), (III-1), (III-2), (IV-1), or (IV-2), X5 is Y, F3G, 3Py6COO,4Py2NH2, 3Py5COO, F3COO, 3Py6NHAc, F, F4C, F40Me, F4COO, Nal2, F3aao, F4aa, F4aao, 3Py6NHaa, 5Pdo, F3CON, F4F, F40Et, F4Me, F4CON, F4CONPEG4Me, F30Me, F3CON, Yae, YaeCOpipzaa, F4aaopipzaa, 4Pdo, 3Py6CON, Alp, Cha4cH, Cha4tH. Cha4cOMe, A1mor, or F4amCOpipzaa, each of which is optionally substituted. In some embodiments, X5 is optionally substituted Y. In some embodiments, X5 is optionally substituted F3G. In some embodiments, X5 is optionally substituted 3Py6COO. In some embodiments, X5 is optionally substituted 4Py2NH2. In some embodiments, X5 is optionally substituted 3Py5COO. In some embodiments, X5 is optionally substituted F3COO. In some embodiments, X5 is optionally substituted 3Py6NHAc. In some embodiments, X5 is optionally substituted F. In some embodiments, X5 is optionally substituted F4C. In some embodiments, X5 is optionally substituted F40Me. In some embodiments, X5 is optionally substituted F4COO. In some embodiments, X5 is optionally substituted Nal2. In some embodiments, X5 is optionally substituted F3aao. In some embodiments, X5 is optionally substituted F4aa. In some embodiments, X5 is optionally substituted F4aao. In some embodiments, X5 is optionally substituted 3Py6NHaa. In some embodiments, X5 is optionally substituted 5Pdo. In some embodiments, X5 is optionally substituted F3CON. In some embodiments, X5 is optionally substituted F4F. In some embodiments, X5 is optionally substituted F40Et. In some embodiments, X5 is optionally substituted F4Me. In some embodiments, X5 is optionally substituted F4CON. In some embodiments, X5 is optionally substituted F4CONPEG4Me. In some embodiments, X5 is optionally substituted F30Me. In some embodiments, X5 is optionally substituted F3CON. In some embodiments, X5 is optionally substituted Yae. In some embodiments, X5 is optionally substituted YaeCOpipzaa. In some embodiments, X5 is optionally substituted F4aaopipzaa. In some embodiments, X5 is optionally substituted 4Pdo. In some embodiments, X5 is optionally substituted 3Py6CON. In some embodiments, X5 is optionally substituted Atp. In some embodiments, X5 is optionally substituted Cha4cH. In some embodiments, X5 is optionally substituted Cha4tH. In some embodiments, X5 is optionally substituted Cha4cOMe. In some embodiments, X5 is optionally substituted A1mor. In some embodiments. X5 is optionally substituted F4amCOpipzaa.

In some embodiments of a peptide of Formula (I), (I-1), (I-2), (I-3), (I-4), (I-5), (II-1), (III-2), (IV-1), or (IV-2), X5 is any amino acid. In some embodiments, X5 is a D-amino acid. In some embodiments, X5 is an L-amino acid. In some embodiments, X5 is an N-alkylated amino acid. In some embodiments, X5 is an N-methylated amino acid. In some embodiments. X5 does not comprise a cyclic group. In some embodiments, X5 comprises a cyclic group. In some embodiments, X5 comprises a 5-6 membered heterocycloalkyl group. In some embodiments, X5 comprises a 5-6 membered heteroaryl group. In some embodiments, X5 comprises a phenyl group. In some embodiments, X5 is a peptoid.

In some embodiments of a peptide of Formula (I), (I-1), (I-2), (I-3), (I-4), (I-5), (III-1), (I-2), (IV-1), or (IV-2), X5 has a structure of

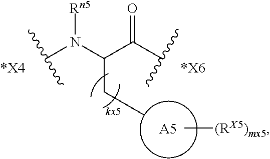

wherein:
Rⁿ⁵ is hydrogen or C₁₋₃ alkyl;
ring A5 is an aryl, heteroaryl, cycloalkyl, or heterocycloalkyl;
each R^{X5} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)Rt, —S(═O)₂R^{a}, —S(═O)₂NR^{c}R^{d}, —S(═O)(═NR^{a})R^{a}, —N═S(═O)NR^{c}R^{d}, —NR^{a}S(═O)₂R^{a}, amidinyl, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}. —NR^{a}C(═O)OR^{a}, —R^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X5}-heterocycloalkyl, -L^{X5}-cycloalkyl, -L^{X5}-aryl, or -L^{X5}-heteroaryl, wherein the alkyl, heteroalkyl, aryl, heteroaryl, cycloalkyl, and heterocycloalkyl are optionally substituted with one or more R^{X5a}; or
two R^{X5} are taken together to form ═O, ═S, or N(R^{a});
L^{X5} is C₁₋₆alkylene, C₁₋₆heteroalkylene, —O—, —S—, or —NR^{a}—, wherein the alkylene and heteroalkylene is optionally substituted with one or more R^{X8a};
kx5 is 0, 1, 2, or 3;
mx5 is 0, 1, 2, 3, 4, or 5;
*X4 represents the point of attachment to X4;
*X6 represents the point of attachment to X6; and other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (II-1), (III-2), (IV-1), or (IV-2), wherein:
Rⁿ⁵ is hydrogen or methyl;
ring A5 is a C₆₋₁₀aryl, 5-10 membered heteroaryl, C₆₋₁₀cycloalkyl, or 5-10 membered heterocycloalkyl;
each R^{X5} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂NR^{c}R^{d}, —S(═O)(═NR)R^{a}, —N═S(═O)NR^{c}R^{d}, —NR^{a}S(═O)₂R^{a}, amidinyl, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, C₆₋₁₀aryl, 5-10 membered heteroaryl, C₃₋₆cycloalkyl, 5-6 membered heterocycloalkyl, -L^{X5}-5-6 membered heterocycloalkyl, -L^{X5}-C₃₋₆cycloalkyl, -L^{X5}-C₆₋₁₀aryl, or -L^{X5}-5-10 membered heteroaryl, wherein the alkyl, heteroalkyl, aryl, heteroaryl, cycloalkyl, and heterocycloalkyl are optionally substituted with one or more R^{X5a}; or
two R^{X5} are taken together to form ═O; and
L^{X5} is C₁₋₆alkylene or C₁₋₆heteroalkylene, wherein the alkylene and heteroalkylene is optionally substituted with one or more R^{X5a};

In some embodiments of a peptide of Formula (I), (I-1), (I-2), (I-3), (I-4), (I-5), (III), (III-2), (TV-1), or (IV-2), wherein:
ring A5 is a phenyl, naphthyl, pyridinyl, cyclohexyl, piperidinyl, piperazinyl, morpholinyl, or tetrahydropyranyl;
each R^{X5} is independently halogen, C₁₋₆alkyl, C₁₋₆haloalkyl, C₁₋₆hydroxyalkyl, C₁₋₆aminoalkyl, C₁₋₆heteroalkyl, —CN, —NO₂, —OR^{a}, —SR^{a}, —NR^{c}R^{d}, —S(═O)R^{a}, —S(═O)₂R², —S(═O)₂NR^{c}R^{d}, —NR^{a}S(═O)₂R^{c}, —NR^{a}C(═NH)NR^{c}R^{d}, —NR^{a}S(═O)₂NR^{c}R^{d}, —C(═O)R^{a}, —C(═O)OR^{a}, —OC^{a}(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, -L^{X5}-piperidinyl, or L^{X5}-piperazinyl, wherein each of the alkyl, heteroalkyl, piperidinyl, and piperazinyl are optionally substituted with one or more R^{X5a}; or
two R^{X5} are taken together to form ═O;
kx5 is 1 or 2; and
mx5 is 0, 1, or 2.

In some embodiments, Rⁿ⁵ is hydrogen. In some embodiments, Rⁿ⁵ is C₁₋₃ alkyl. In some embodiments, Rⁿ⁵ is methyl.

In some embodiments, ring A5 is a C₆₋₁₀aryl, 5-10 membered heteroaryl, C₃₋₁₀cycloalkyl, or 3-10 membered heterocycloalkyl. In some embodiments, ring A5 is a C₆₋₁₀aryl, 5-10 membered heteroaryl, C₆₋₁₀cycloalkyl, or 5-10 membered heterocycloalkyl. In some embodiments, ring A5 is phenyl, naphthyl, pyridinyl, cyclohexyl, piperidinyl, piperazinyl, morpholinyl, or tetrahydropyranyl. In some embodiments, ring A5 is C₆₋₁₀aryl. In some embodiments, ring A5 is phenyl. In some embodiments, ring A5 is naphthyl. In some embodiments, ring A5 is 5-10 membered heteroaryl. In some embodiments, ring A5 is 6-10 membered heteroaryl. In some embodiments, ring A5 is 5-6 membered heteroaryl. In some embodiments, ring A5 is pyridine. In some embodiments, ring A5 is pyridine. In some embodiments, ring A5 is C₃₋₁₀cycloalkyl. In some embodiments, ring A5 is C₅₋₁₀cycloalkyl. In some embodiments, ring A5 is C₅₋₆ cycloalkyl. In some embodiments, ring A5 is C₃₋₆cycloalkyl. In some embodiments, ring A5 is cyclohexane. In some embodiments, ring A5 is 3-10 membered heterocycloalkyl. In some embodiments, ring A5 is 5-6 membered heterocycloalkyl. In some embodiments, ring A5 is tetrahydropyran. In some embodiments, ring A5 is morpholine.

In some embodiments, each R^{X5} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a},—SR^{a},—SF₅, —NR^{c}R^{d}, —S(═O)R^{c}, —S(═O)₂R^{a}, —S(═O)₂NR^{c}R^{a}, —S(═O)(═NR^{a})R^{a}, —N═S(═O)NR^{c}R^{d}, —NR^{a}S(═O)₂R^{a}, amidinyl, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{a}), —P(═O)R^{c}R^{d}, C₆₋₁₀aryl, 5-10 membered heteroaryl, C₃₋₆cycloalkyl, 5-6 membered heterocycloalkyl, -L^{X5}-5-6 membered heterocycloalkyl, -L^{X5}-C₃₋₆cycloalkyl, -L^{X5}-C₆₋₁₀aryl, or -L^{X5}-5-10 membered heteroaryl, wherein the alkyl, heteroalkyl, aryl, heteroaryl, cycloalkyl, and heterocycloalkyl are optionally substituted with one or more R^{X5a}. In some embodiments, each R^{X5} is independently C₁₋₆alkyl, C₁₋₆haloalkyl, C₁₋₆hydroxy alkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —NR^{c}R^{d}, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —NR^{a}C(═O)R^{a}, —NR^{a}C(═O)OR^{a}, —C(═O)NR^{c}R^{d}, C₆₋₁₀aryl, 5-10 membered heteroaryl, C₃₋₆cycloalkyl, 5-6 membered heterocycloalkyl, -L^{X5}-5-6 membered heterocycloalkyl, -L^{X5}-C₃₋₆cycloalkyl, -L^{X5}-C₆₋₁₀aryl, or -L^{X5}-5-10 membered heteroaryl, wherein the alkyl, heteroalkyl, aryl, heteroaryl, cycloalkyl, and heterocycloalkyl are optionally substituted with one or more R^{X3a}. In some embodiments, each R^{X5} is independently halogen, C₁₋₆alkyl, C₁₋₆haloalkyl, C₁₋₆hydroxyalkyl, C₁₋₆aminoalkyl, C₁₋₆heteroalkyl, —CN, —NO₂, —OR^{a}, —SR^{a}, —NR^{c}R^{d}, —S(═O)R^{c}, —S(═O)₂R^{a}, —S(═O)NR^{c}R^{d}, —NR^{a}S(═O)₂R^{c}, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, -L^{X5}-piperidinyl, or L^{X5}-piperazinyl, wherein each of the alkyl, heteroalkyl, piperidinyl, and piperazinyl are optionally substituted with one or more R^{X5a}.

In some embodiments, each R^{X5a} is independently halogen, C₁₋₆alkyl, C₁-C₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR)₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments, two R^{X5} are taken together to form ═O.

In some embodiments, kx5 is 0. In some embodiments, kx5 is 1. In some embodiments, kx5 is 2. In some embodiments, kx5 is 3.

In some embodiments, mx5 is 0. In some embodiments, mx5 is 1. In some embodiments, mx5 is 2. In some embodiments, mx5 is 3. In some embodiments, mx5 is 4. In some embodiments, mx5 is 5.

In some embodiments. Rn5 is hydrogen, kx5 is 1 or 2, mx5 is 0, 1, or 2, ring A5 is phenyl, and each R^{X5} is independently halogen, C₁₋₆alkyl, C₁₋₆haloalkyl, or —C(═O)OR^{a}. In some embodiments, Rn5 is hydrogen, kx5 is 1, mx5 is 1, ring A5 is phenyl, and R^{X5} is —C(═O)OH.

In some embodiments of Formula (I), (I-1), (I-2), (I-3), (I-4), (I-5), (II-1), (III-2), (IV-1), or (IV-2), X6 is an aliphatic amino acid (e.g., V, L, I, A, G, or a variant thereof), a hydrophilic amino acid (e.g., D, E, or a variant thereof), threonine (T), serine (S), O-methyl threonine (TMe), or methionine (M).

In some embodiments of Formula (I), (I-1), (I-2), (I-3), (I-4), (I-5), (III-1), (III-2), (IV-1), or (IV-2), X6 is 1, V, Eva, Chg, Tbg, A, L, Ahp, F4COO, Gcpr, Gcpe, alI, Cle, S3REt, TMe, Acpr, Cba, Gthp, NleCOO, NeOH, P, Atb, Nva, Nle, N, DapAc, Abu, Nmm, Ndm, Neit, Cit, SMe, HseMe, HseEt, HseiPr, dMeS, TdMe, Cbg, NvaOMe, SiPr, Spr, NIeOMe, Sbu, Scbm, Scpe, AhpOMe, HseBu, Spent or Hsecpe. In some embodiments of Formula (I), (I-1), (I-2), (I-3), (I-4), (I-5), (III-1), (II-2), (IV-1), or (IV-2), X6 is I, V, Eva, Chg, Tbg, A, L, Ahp, F4COO, Gcpr, Gcpe, alI, Cle, S3REt, TMe, Acpr, Cba, Gthp, NleCOO, NIeOH, P, Atb, Nva, Nle, N, DapAc, Abu, Nmm, Ndm, Ncit, Cit, SMe, HseMe, HseEt, HseiPr, dMeS, TdMe, Cbg, NyaOMe, SiPr, Spr, NeOMe, Sbu, Scbm, Scpe, AhpOMe, HseBu, Spent or Hsecpe, each of which is optionally substituted. In some embodiments, X6 is optionally substituted I. In some embodiments, X6 is optionally substituted V. In some embodiments, X6 is optionally substituted Eva. In some embodiments, X6 is optionally substituted Chg. In some embodiments, X6 is optionally substituted Tbg. In some embodiments, X6 is optionally substituted A. In some embodiments, X6 is optionally substituted L. In some embodiments, X6 is optionally substituted Ahp. In some embodiments, X6 is optionally substituted F4COO. In some embodiments, X6 is optionally substituted Gcpr. In some embodiments, X6 is optionally substituted Gcpe. In some embodiments, X6 is optionally substituted all. In some embodiments, X6 is optionally substituted Cle. In some embodiments, X6 is optionally substituted S3REt. In some embodiments, X6 is optionally substituted TMe. In some embodiments, X6 is optionally substituted Acpr. In some embodiments, X6 is optionally substituted Cba. In some embodiments, X6 is optionally substituted Gthp. In some embodiments, X6 is optionally substituted NIeCOO. In some embodiments, X6 is optionally substituted NleOH. In some embodiments, X6 is optionally substituted P. In some embodiments, X6 is optionally substituted Atb. In some embodiments, X6 is optionally substituted Nva. In some embodiments, X6 is optionally substituted NIe. In some embodiments, X6 is optionally substituted N. In some embodiments, X6 is optionally substituted DapAc. In some embodiments, X6 is optionally substituted Abu. In some embodiments. X6 is optionally substituted Nmm. In some embodiments, X6 is optionally substituted Ndm. In some embodiments, X6 is optionally substituted Ncit. In some embodiments. X6 is optionally substituted Cit. In some embodiments, X6 is optionally substituted SMe. In some embodiments, X6 is optionally substituted HseMe. In some embodiments, X6 is optionally substituted HseEt. In some embodiments. X6 is optionally substituted HseiPr. In some embodiments. X6 is optionally substituted dMeS. In some embodiments, X6 is optionally substituted TdMe. In some embodiments, X6 is optionally substituted Cbg. In some embodiments. X6 is optionally substituted NvaOMe. In some embodiments, X6 is optionally substituted SiPr. In some embodiments, X6 is optionally substituted Spr. In some embodiments, X6 is optionally substituted NcOMe. In some embodiments, X6 is optionally substituted Sbu. In some embodiments. X6 is optionally substituted Scbm. In some embodiments, X6 is optionally substituted Sepe. In some embodiments, X6 is optionally substituted AhpOMe. In some embodiments, X6 is optionally substituted HseBu. In some embodiments, X6 is optionally substituted Spent. In some embodiments, X6 is optionally substituted Hsecpe.

In some embodiments of a peptide of Formula (I), (I-1), (I-2), (I-3), (I-4), (I-5), (III-1), (III-2), (IV-1), or (IV-2), X6 is any amino acid. In some embodiments, X6 is a D-amino acid. In some embodiments, X6 is an L-amino acid. In some embodiments, X6 is an N-alkylated amino acid. In some embodiments, X6 is an N-methylated amino acid. In some embodiments, X6 does not comprise a cyclic group. In some embodiments, X6 comprises a cyclic group. In some embodiments, X6 comprises a 5-6 membered heterocycloalkyl group. In some embodiments, X6 comprises a 5-6 membered heteroaryl group. In some embodiments, X6 comprises a phenyl group. In some embodiments, X6 is a peptoid.

In some embodiments of a peptide of Formula (I), (I-1), (I-2), (I-3), (I-4), (I-5), (III-1), (III-2), (IV-1), or (IV-2), X6 has a structure of

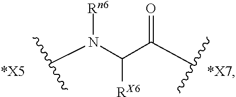

wherein:
Rⁿ⁶ is hydrogen or C₁₋₃ alkyl;
R^{X6} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, aryl, heteroaryl, cycloalkyl, heterocycloalkyl, -L^{X6}-heterocycloalkyl, -L^{X6}-cycloalkyl, -L^{X6}-aryl, or -L^{X6}-heteroaryl, wherein each of the alkyl, heteroalkyl, alkenyl, alkynyl, aryl, heteroaryl, cycloalkyl, and heterocycloalkyl is optionally substituted with one or more R^{X6a}; or
Rⁿ⁶ and R^{X6} are taken together with the intervening atoms to form a 5-to 6-membered heterocycloalkyl, which is optionally substituted with one or more R^{X6a}.
L^{X6} is C₁₋₆alkylene, C₁₋₆heteroalkylene. O, S, or NR^{a}, wherein the alkylene and heteroalkylene is optionally substituted with one or more R^{X6a};
*X5 represents the point of attachment to X5;
*X7 represents the point of attachment to X7; and
other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (II-1), (III-2), (IV-1), or (IV-2), wherein
Rⁿ⁶ is hydrogen or methyl;
R^{X6} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alknyl, C₆₋₁₀aryl, 5-10 membered heteroaryl, C₃₋₆cycloalkyl, 5-6 membered heterocycloalkyl, -L^{X6}-5-6 membered heterocycloalkyl, -L^{X6}-C₃₋₆cycloalkyl, -L^{X6}-C₆₋₁₀aryl, or -L^{X6}-5-10 membered heteroaryl, wherein each of the alkyl, heteroalkyl, alkenyl, alkynyl, aryl, heteroaryl, cycloalkyl, and heterocycloalkyl is optionally substituted with one or more R^{X6a}; and
L^{X6} is C₁₋₆alkylene or C₁₋₆heteroalkylene, wherein the alkylene and heteroalkylene is optionally substituted with one or more R^{X6a}.

In some embodiments, Rⁿ⁶ is hydrogen. In some embodiments, Rⁿ⁶ is C₁₋₃ alkyl. In some embodiments, Rⁿ⁶ is methyl.

In some embodiments, R^{X6} is C₁₋₆alkyl, C₁₋₆haloalkyl, C₁₋₆hydroxyalkyl, C₁₋₆aminoalkyl, C₁₋₆heteroalkyl, C₃₋₆cycloalkyl, 5-6 membered heterocycloalkyl, -L^{X6}-5-6 membered heterocycloalkyl, -L^{X6}-C₃₋₆cycloalkyl, -L^{X6}-phenyl or L^{X6}-6 membered heteroaryl, wherein each of the alkyl, heteroalkyl, phenyl, heteroaryl, cycloalkyl, and heterocycloalkyl is optionally substituted with one or more R^{X6a}.

In some embodiments, R^{X6} is C₁₋₆alkyl, C₁₋₆haloalkyl, C₁₋₆hydroxyalkyl, C₁₋₆aminoalkyl, C₁₋₆heteroalkyl, -L^{X6}-5-6 membered heterocycloalkyl, -L^{X6}-C₃₋₆-cycloalkyl, -L^{X6}-C₆₋₁₀aryl, or -L^{X6}-5-10 membered heteroaryl, wherein each of the alkyl, heteroalkyl, aryl, heteroaryl, cycloalkyl, and heterocycloalkyl is optionally substituted with one or more R^{X6}C.

In some embodiments, R^{X6} is C₁₋₆alkyl, C₁₋₆haloalkyl, C₁₋₆hydroxyalkyl, C₁₋₆aminoalkyl, C₁₋₆heteroalkyl, -L^{X6}-5-6 membered heterocycloalkyl, -L^{X6}-C₄₋₆cycloalkyl, -L^{X6}-C₆₋₁₀aryl, or -L^{X6}-5-10 membered heteroaryl, wherein each of the alkyl, heteroalkyl, aryl, heteroaryl, cycloalkyl, and heterocycloalkyl is optionally substituted with one or more R^{X6a}; In some embodiments, R^{X6} is C₁₋₆alkyl, C₁₋₆heteroalkyl, 6-L^{X6}-phenyl, or -L^{X6}-pyridinyl, wherein each of the alkyl, heteroalkyl, phenyl, and pyridinyl is optionally substituted with one or more R^{X6a}.

In some embodiments, R^{X6} is optionally substituted C₁₋₆alkyl. In some embodiments, R^{X6} is C₁₋₆alkyl optionally substituted with one or more R^{X6a}. In some embodiments, R^{X6} is optionally substituted C₁₋₆heteroalkyl. In some embodiments, R^{X6} is C₁₋₆heteroalkyl optionally substituted with one or more R^{X6a}. In some embodiments, R^{X6a} is C₁₋₆alkyl, C₁₋₆haloalkyl, C₁₋₆hydroxyalkyl, C₁₋₆aminoalkyl, or C₁₋₆heteroalkyl, wherein each of which is optionally substituted with one or more R^{X6a}; In some embodiments, R^{X6} is C₂₋₆alkenyl or C₂₋₆alkynyl, wherein each of which is optionally substituted with one or more R^{X6a}. In some embodiments, R^{X6} is aryl, heteroaryl, cycloalkyl, or heterocycloalkyl, wherein each of which is optionally substituted with one or more R^{X6a}; In some embodiments, R^{X6} is aryl or heteroaryl, wherein each of which is optionally substituted with one or more R^{X6a}. In some embodiments, R^{X6} is cycloalkyl or heterocycloalkyl, wherein each of which is optionally substituted with one or more R^{X6}. In some embodiments, R^{X6} is C₃ cycloalkyl, which is optionally substituted with one or more R^{X6a}. In some embodiments, R^{X6} is -L^{X6}-heterocycloalkyl, -L^{X6}-cycloalkyl, -L^{X6}-aryl, or -L^{X6}-heteroaryl, wherein each of which is optionally substituted with one or more R^{X6a}. In some embodiments, R^{X6} is -L^{X6}-heterocycloalkyl, which is optionally substituted with one or more R^{X6a}. In some embodiments. R^{X6} is -L^{X6}-cycloalkyl, which is optionally substituted with one or more R^{X6a}; In some embodiments, R^{X6} is -L^{X6}-aryl, which is optionally substituted with one or more R^{X6a}; In some embodiments. R^{X6} is -L^{X6}-heteroaryl, which is optionally substituted with one or more R^{X6a}.

In some embodiments. Rⁿ⁶ and R^{X6} are taken together with the intervening atoms to form a 5-to 6-membered heterocycloalkyl, which is optionally substituted with one or more R^{X6a}.

In some embodiments, each R^{X6a} is independently halogen, C₁₋₆ alkyl, C₁-C₆haloalkyl, C₁-C₆hydroxyalkyl, C₁-C₆aminoalkyl, C₁-C₆heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{c}, —NR^{a}C(═O)NR^{c}R^{d}, —C(═O)NR^{c}R^{d}, or ═O, wherein each of the alkyl and heteroalkyl is optionally substituted with one or more R^{e}.

In some embodiments. Rⁿ⁶ is hydrogen, and R^{X6} is C₁₋₆alkyl which is optionally substituted with one or more R^{X6a}. In some embodiments, each R^{X6a} is independently halogen, C₁₋₆ alkyl, C₁-C₆haloalkyl, C₁-C₆hydroxyalkyl, C₁-C₆aminoalkyl, C₁-C₆heteroalkyl, or —OR^{a}. In some embodiments, each R^{X6a} is independently halogen, or —O—C₁₋₆ alkyl. In some embodiments, R^{X6a} is —O—CH₃. In some embodiments, R^{X6} is —CH(CH₃)OCH₃.

In some embodiments of Formula (I), (I-1), (I-2), (I-3), (I-4), (I-5), (III-1), (III-2), (IV-1), or (IV-2), X7 is an amino acid comprising a branched alkyl side chain, a C₃₋₅cycloalkyl side chain, or a 3-to 5-membered heterocycloalkyl side chain, or an N-methylated variant thereof. In some embodiments, the branched alkyl side chain comprises 3-5 carbon atoms. In some embodiments, the alkyl, cycloalkyl, or heterocycloalkyl side chain is optionally substituted with —O—C₁-C₆ alkyl.

In some embodiments of Formula (I), (I-1), (I-2), (I-3), (I-4), (I-5), (III-1), (III-2), (IV-1), or (IV-2), X7 is I, Eva, alI, TMe, SMe, Gcpr, Gepe, Gthp, dMeS, TdMe, or Cbg. In some embodiments of Formula (I), (I), (I-2), (I-3), (I-4), (I-5), (III-1), (II-2), (IV-1), or (IV-2). X7 is L Eva, alI, TMe, SMe, Gcpr, Gcpe, Gthp, dMeS, TdMe, or Cbg, each of which is optionally substituted. In some embodiments, X7 is optionally substituted I. In some embodiments, X7 is optionally substituted Eva. In some embodiments, X7 is optionally substituted all. In some embodiments, X7 is optionally substituted TMe. In some embodiments, X7 is optionally substituted SMe. In some embodiments, X7 is optionally substituted Gcpr. In some embodiments, X7 is optionally substituted Gcpe. In some embodiments, X7 is optionally substituted Gthp. In some embodiments, X7 is optionally substituted dMeS. In some embodiments, X7 is optionally substituted TdMe. In some embodiments, X7 is optionally substituted Cbg.

In some embodiments of a peptide of Formula (I), (I-1), (I-2), (I-3), (I-4), (I-5), (III-1), (III-2), (IV-1), or (IV-2), X7 is any amino acid. In some embodiments, X7 is a D-amino acid. In some embodiments. X7 is an L-amino acid. In some embodiments, X7 is an N-alkylated amino acid. In some embodiments, X7 is an N-methylated amino acid. In some embodiments, X7 does not comprise a cyclic group. In some embodiments, X7 comprises a cyclic group. In some embodiments. X7 comprises a 5-6 membered heterocycloalkyl group. In some embodiments, X7 comprises a 5-6 membered heteroaryl group. In some embodiments, X7 comprises a phenyl group. In some embodiments, X7 is a peptoid.

In some embodiments of a peptide of Formula (I), (I-1), (I-2), (I-3), (I-4), (I-5), (III-1), (III-2), (IV-1), or (IV-2), X7 has a structure of

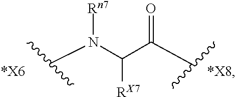

wherein:
Rⁿ⁷ is hydrogen or C₁₋₃ alkyl;
R^{X7} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, cycloalkyl, or heterocycloalkyl, wherein each of the alkyl, heteroalkyl, alkenyl, alkynyl, cycloalkyl, and heterocycloalkyl is optionally substituted with one or more R^{X7a};
*X6 represents the point of attachment to X6; and
*X8 represents the point of attachment to X8;
other groups (such as R^{a}, R^{c}, R^{d}, and R^{e}) have the meanings defined in Formula (I).

In some embodiments, wherein Rⁿ⁷ is hydrogen or methyl; and R^{X7} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, C₃₋₆cycloalkyl, or 5-6 membered heterocycloalkyl, wherein each of the alkyl, heteroalkyl, alkenyl, alkynyl, cycloalkyl, and heterocycloalkyl is optionally substituted with one or more R^{X7a}.

In some embodiments, Rⁿ⁷ is hydrogen. In some embodiments, Rⁿ⁷ is C₁₋₃ alkyl. In some embodiments, Rⁿ⁷ is methyl.

In some embodiments, R^{X7} is C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, C₃₋₆cycloalkyl, or 5-6 membered heterocycloalkyl, wherein each of the alkyl, heteroalkyl, alkenyl, alkynyl, cycloalkyl, and heterocycloalkyl is optionally substituted with one or more R^{X7a}. In some embodiments, R^{X7} is C₁₋₆alkyl, C₁₋₆heteroalkyl, C₃₋₆cycloalkyl, or 5-6 membered heterocycloalkyl, wherein each of the alkyl, heteroalkyl, cycloalkyl, and heterocycloalkyl is optionally substituted with one or more R^{X7}. In some embodiments, R^{X7} is C₁₋₆alkyl, which is optionally substituted with one or more R^{X7a}. In some embodiments, R^{X7} is C₁₋₆alkyl. In some embodiments, R^{X7} is a branched C₁₋₆alkyl. In some embodiments, R^{X7} is C₁₋₆heteroalkyl, which is optionally substituted with one or more R^{X7a}. In some embodiments, R^{X7} is C₁₋₆heteroalkyl. In some embodiments, R^{X7} is C₃₋₆cycloalkyl, which is optionally substituted with one or more R^{X7}. In some embodiments, R^{X7} is C₃₋₆cycloalkyl. In some embodiments, R^{X7} is 5-6 membered heterocycloalkyl, which is optionally substituted with one or more R^{X7} In some embodiments, R^{X7} is 5-6 membered heterocycloalkyl.

In some embodiments, each R^{X7} is independently halogen, —CN, —OR^{a}, —SR^{a}, —NR^{c}R^{d}, —C(═O)R^{a}, —C(═O)OR^{a}, —OC(═O)R², —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or ═O.

In some embodiments, Rⁿ⁷ is hydrogen and R^{X7} is C₁₋₆alkyl, which is optionally substituted with one or more R^{X7a}. In some embodiments. Rⁿ⁷ is hydrogen and R^{X7} is C₁₋₆alkyl. In some embodiments, Rⁿ⁷ is hydrogen and R^{X7} is C₃₋₆alkyl. In some embodiments, Rⁿ⁷ is hydrogen and R^{X7} is branched C₁₋₆alkyl.

In some embodiments of Formula (I), (I-1), (I-2), (I-3), (I-4), (I-5), (III-1), (III-2), (IV-1), or (IV-2), X8 is Y. or a variant thereof comprising a hydroxyphenyl ring, wherein the hydroxy phenyl ring of Y or of the variant is optionally substituted with one or more substituents selected from halogen, —C₁₋₃alkyl, —OH, —C(═O)OH, —O—CH₃, —C₁₋₃alkylene-C(═O)OH, —C₁₋₃alkylene-C(═O)-5-to 6-membered heterocycloalkylene-C₁₋₃alkylene-C(═O)OH, and —C₁₋₆alkylene-NHC(═O)-5-to 6-membered heterocycloalkylene-C₁₋₆alkylene-C(═O)OH. In some embodiments of Formula (I), (I-1), (I-2), (I-3), (I-4), (I-5), (III-1), (III-2), (IV-1), or (IV-2), X8 is F. or a variant thereof comprising a phenyl, pyridinyl, or indazolyl, wherein the phenyl of F or the phenyl, pyridinyl, or indazolyl of the variant is optionally substituted with one or more substituents each independently selected from halogen, —C₁₋₃alkyl, —OH, —C(═O)OH, —C(═O)NH₂, —NHC(═O)NH₂, —C₁₋₃alkylene-C(NH₂)—COOH, —NH—CO—CH₃, —NH—C₁₋₃alkylene-NH₂, —C(═O)—N(CH₂)₂, —S(═O)₂—CH₃, —C₁₋₃alkylene-NH—C(═O)— 5-to 6-membered heterocycloalkylene-C₁₋₃alkylene-C(═O)OH, and —Oμ-C₁₋₃alkylene-NH—C(═O)-5-to 6-membered heterocycloalkylene-C₁₋₃alkylene-C(═O)OH.

In some embodiments of Formula (I), (I-1), (I-2), (I-3), (I-4), (I-5), (III-1), (III-2), (IV-1), or (IV-2), X8 is Y, F40Me, F4COO, F40Et, F4u, F4Me, F4CONdMe, F4CON, F4 ms, F4CONPEG4Me, F34dOMe, F3OMe, F3C, F3CON, F3CONdMe, 3Py6CON, Yac, YaeCOpipzaa, 5inda, F3aao, F3aa. F4aao, F4aa, 3Py6Nhae, 3Py6NHAc, 3Py6OMe, F4aaopipzaa, or F4amCOpipzaa. In some embodiments of Formula (I), (I-1), (I-2), (I-3), (I-4), (I-5), (III-1), (III-2), (IV-1), or (IV-2). X8 is Y. F40Me, F4COO, F40Et, F4u, F4Me, F4CONdMe, F4CON, F4 ms, F4CONPEG4Me, F34dOMe, F30Me, F3C, F3CON, F3CONdMe, 3Py6CON, Yac, YaeCOpipzaa, 5Inda, F3aao, F3aa, F4aao, F4aa, 3Py6Nhae, 3Py6NIAc, 3Py6OMe, F4aaopipzaa, or F4amCOpipzaa, each of which is optionally substituted. In some embodiments, X8 is optionally substituted Y. In some embodiments, X8 is optionally substituted F40Me. In some embodiments, X8 is optionally substituted F4COO. In some embodiments, X8 is optionally substituted F40Et. In some embodiments, X8 is optionally substituted F4u. In some embodiments, X8 is optionally substituted F4Me. In some embodiments. X8 is optionally substituted F4CONdMe. In some embodiments, X8 is optionally substituted F4CON. In some embodiments, X8 is optionally substituted F4 ms. In sonie embodiments, X8 is optionally substituted F4CONPEG4Me. In some embodiments. X8 is optionally substituted F34dOMe. In sonie embodiments, X8 is optionally substituted F30Me. In sonie embodiments, X8 is optionally substituted F3C. In some embodiments, X8 is optionally substituted F3CON. In some embodiments, X8 is optionally substituted F3CONdMe. In some embodiments, X8 is optionally substituted 3Py6CON. In some embodiments, X8 is optionally substituted Yac. In some embodiments, X8 is optionally substituted YaeCOpipzaa. In some embodiments. X8 is optionally substituted 5Inda. In some embodiments, X8 is optionally substituted F3aao. In some embodiments, X8 is optionally substituted F3aa. In some embodiments, X8 is optionally substituted F4aao. In some embodiments, X8 is optionally substituted F4aa. In some embodiments, X8 is optionally substituted 3Py6Nhae. In some embodiments, X8 is optionally substituted 3Py6NHAc. In some embodiments, X8 is optionally substituted 3Py6OMe. In some embodiments, X8 is optionally substituted F4aaopipzaa. In some embodiments, X8 is optionally substituted F4amCOpipzaa.

In some embodiments of Formula (I), (I-1), (I-2), (I-3), (I-4), (I-5), (III-1), (III-2), (IV-1), or (IV-2), X8 is A, I, L, or V. In some embodiments, X8 is A. In some embodiments. X8 is A or a derivative thereof. In some embodiments, X8 is I or a derivative thereof. In some embodiments, X8 is L or a derivative thereof. In some embodiments, X8 is V or a derivative thereof.

In some embodiments of a peptide of Formula (I), (I-1), (I-2), (I-3), (I-4), (I-5), (III-1), (III-2), (IV-1), or (IV-2), X8 is any amino acid. In some embodiments, X8 is a D-amino acid. In some embodiments, X8 is an L-amino acid. In some embodiments, X8 is an aliphatic-amino acid. In some embodiments, X8 is an N-alkylated amino acid. In some embodiments, X8 is an N-methylated amino acid. In some embodiments, X8 does not comprise a cyclic group. In some embodiments, X8 comprises a cyclic group. In some embodiments, X8 comprises a 5-6 membered heterocycloalkyl group. In some embodiments, X8 comprises a 5-6 membered heteroaryl group. In some embodiments, X8 comprises a phenyl group. In some embodiments, X8 is a peptoid.

In some embodiments of a peptide of Formula (I), (I-1), (I-2), (I-3), (I-4), (I-5), (II-1), (I-2), (IV-1), or (IV-2), X8 has a structure of

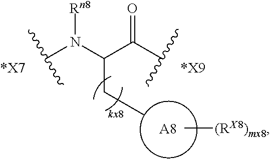

wherein,
Rⁿ⁸ is hydrogen or C₁₋₃alkyl;
ring A8 is an aryl or heteroaryl;
each R^{X8} is independently halogen, C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, —CN, —NO₂, —OR^{a}, —SR^{a}, —SF₅, —NR^{c}R^{d}, —S(═O)R^{a}, —S(═O)₂R^{a}, —S(═O)₂R^{c}R^{a}, —S(═O)(═NR^{a})R^{a}, —N═S(═O)R^{a}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{c}, —OC(═O)NR^{c}R^{d}, —NR^{a}C(═O)R^{a}, —NR^{a}C(═O)OR^{a}, —NR^{a}C(═O)NR^{c}, —C(═O)NR^{c}R^{d}, —P(═O)(OR^{a})(OR^{a}), —P(═O)R^{c}R^{d}, aryl, heteroaryl, cycloalkyl, heterocycloalkyl, -L^{X8}-heterocycloalkyl, -L^{X8}-cycloalkyl, -L^{X8}-aryl, or -L^{X8}-heteroaryl, wherein each of the alkyl, heteroalkyl, alkenyl, alkynyl, aryl, heteroaryl, cycloalkyl, and heterocycloalkyl is optionally substituted with one or more R^{X8a}; or
two R^{X8} are taken together to form ═O, ═S, or ═N(R^{a});
L^{X8} is C₁₋₆alkylene, C₁₋₆heteroalkylene, —O—, —S—, or —NR^{a}—, wherein the alkylene and heteroalkylene is optionally substituted with one or more R^{X8a};
kx8 is 0, 1, 2, or 3;
mx8 is 0, 1, 2, 3, 4, or 5;
*X7 represents the point of attachment to X7;
*X9 represents the point of attachment to X9; and
other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (III-1), (III-2), (IV-1), or (IV-2), wherein
ring A8 is a C₆₋₁₀aryl or 5-10 membered heteroaryl;
each R^{X8} is independently C₁₋₆alkyl, C₁₋₆haloalkyl, C₁₋₆hydroxy alkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₅, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NRLS(═O)₂R^{a}, amidinyl, —NR^{c}C(═NH)NR^{c}R^{d}, —NR^{a}S(═O)₂R^{c}R^{d}, —C(═O)R^{a}, —C(═O)OW, —OC(═O)R^{a}, —OC(═O)OR^{a}, —OC(═O)NR^{c}R^{d}, —NR^{a}C(═O)R^{a}, —NR³C(═O)OR^{a}, —NR^{a}CC(═O)NR^{c}R^{d}, —C(═O)NR^{c}R^{d}, —P(═O)(OR^{a})(OR^{a}), —P(═O)R^{c}R^{d}, C₆₋₁₀aryl, 5-10 membered heteroaryl, C₃₋₆cycloalkyl, 5-6 membered heterocycloalkyl, -L^{X8}-5-6 membered heterocycloalkyl, -L^{X6}-C₃₋₆cycloalkyl, -L^{X6}-C₆₋₁₀aryl, or -L^{X6}-5-10 membered heteroaryl, wherein each of the alkyl, heteroalkyl, alkenyl, alkynyl, aryl, heteroaryl, cycloalkyl, and heterocycloalkyl is optionally substituted with one or more R^{X8a}; or
two R^{X8} are taken together to form ═O; and
L^{X8} is C₁₋₆alkylene or C₁₋₆heteroalkylene, wherein the alkylene and heteroalkylene is optionally substituted with one or more R^{X8a}.

In some embodiments of a peptide of Formula (I), (I-1), (I-2), (I-3), (I-4), (I-5), (III-1), (III-2), (IV-1), or (IV-2), wherein
ring A8 is a phenyl, pyridinyl, indolyl, azaindolyl, indazolyl, or benzimidazolyl;
each R^{X8} is independently halogen, C₁₋₆alkyl, C₁₋₆haloalkyl, C₁₋₆hydroxyalkyl, C₁₋₆aminoalkyl, C₁₋₆ heteroalkyl, —CN, —NO₂, —OR^{a}, —SR^{a}, —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, -L^{X5}-piperidinyl, or L^{X5}-piperazinyl, wherein each of the alkyl, heteroalkyl, piperidinyl, and piperazinyl are optionally substituted with one or more R^{X8a}; or
two R^{X5} are taken together to form ═O;
kx8 is 1 or 2; and
mx8 is 0, 1, or 2.

In some embodiments, Rⁿ⁸ is hydrogen. In some embodiments, Rⁿ⁸ is C₁₋₃ alkyl. In some embodiments, Rⁿ⁸ is methyl.

In some embodiments, ring A8 is C₆₋₁₀aryl. In some embodiments, ring A8 is a phenyl. In some embodiments, ring A8 is naphthyl.

In some embodiments, ring A8 is 5-10 membered heteroaryl. In some embodiments, ring A8 is 5-6 membered heteroaryl. In some embodiments, ring A8 is monocyclic heteroaryl. In some embodiments, ring A8 is bicyclic heteroaryl. In some embodiments, ring A8 is bicyclic fused heteroaryl. In some embodiments, ring A8 is bicyclic 5-6 or 6-5 fused heteroaryl. In some embodiments, ring A8 is a phenyl, pyridinyl, indolyl, azaindolyl, indazolyl, or benzimidazolyl.

In some embodiments, each R^{X8} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF^{a}, —NR^{c}R^{d}, —S(═O)R^{a}, —S(═O)₂R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{c}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{a}, —P(═O)(OR^{c})(OR^{d}), —P(═O)R^{c}R^{d}, C₆₋₁₀aryl, 5-10 membered heteroaryl, C₃₋₆cycloalkyl, 5-6 membered heterocycloalkyl, -L^{X5}5-6 membered heterocycloalkyl, -L^{X8}-C₃₋₆cycloalkyl, -L^{X8}-C₆₋₁₀aryl, or -L^{X8}-5-10 membered heteroaryl, wherein each of the alkyl, heteroalkyl, alkenyl, alkynyl, aryl, heteroaryl, cycloalkyl, and heterocycloalkyl is optionally substituted with one or more R^{X8a}.

In some embodiments, each R^{X8} is independently halogen, C₁₋₆alkyl, C₁₋₆haloalkyl, C₁₋₆hydroxyalkyl, C₁₋₆aminoalkyl, C₁₋₆heteroalkyl, —CN, —NO₂, —OR^{a}, —SRW, —NR^{c}R^{d}, —S(═O)R^{a}, —S(═O)₂R, —S(═O)₂NR^{c}R^{d}, —NR^{a}S(═O)₂R^{a}, —NR^{a}C(═NH)NR^{c}R^{d}, —NR^{a}S(═O)₂NR^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, or -L^{X5}5-6 membered heterocycloalkyl (e.g., -L^{X8}-piperidinyl or L^{X8}-piperazinyl), wherein each of the alkyl, heteroalkyl, heterocycloalkyl (e.g., piperidinyl, and piperazinyl) are optionally substituted with one or more R^{X8a}.

In some embodiments, R^{X8} are taken together to form an oxo.

In some embodiments, L^{X8} is C₁₋₆alkylene, which is optionally substituted with one or more R^{X8a}. In some embodiments, L^{X8} is C₁₋₆heteroalkylene, which is optionally substituted with one or more R^{X8a}. In some embodiments. L^{X8} is —O—. In some embodiments, L^{X8} is —S—. In some embodiments, L^{X8} is —NR^{a}—.

In some embodiments, kx8 is 0. In some embodiments, kx8 is 1. In some embodiments, kx8 is 2. In some embodiments, kx8 is or 3.

In some embodiments, mx8 is 0. In some embodiments, mx8 is 1. In some embodiments, mx8 is 2. In some embodiments, mx8 is 3. In some embodiments, mx8 is 4. In some embodiments, mx8 is 5.

In some embodiments, Rⁿ⁸ is hydrogen, kx8 is 0, 1, or 2, mx8 is 0, 1, or 2, ring A8 is phenyl, and each R^{X8} is independently halogen, C₁₋₆alkyl, C₁₋₆haloalkyl, C₁₋₆hydroxyalkyl, C₁₋₆aminoalkyl, C₁₋₆heteroalkyl, or —OR^{a}, wherein each of the alkyl or heteroalkyl is optionally substituted with one or more R^{X8a}. In some embodiments, Rⁿ⁸ is hydrogen, kx8 is 1, mx8 is 1, ring A8 is phenyl, and each R^{X8} is independently halogen, C-alkyl, or —OR^{a}, wherein each of the alkyl or heteroalkyl is optionally substituted with one or more R^{X8a}. In some embodiments, Rⁿ⁸ is hydrogen, kx8 is 1, mx8 is 1, ring A8 is phenyl, and each R^{X8} is independently halogen, C₁₋₆alkyl, or —OCH₂C(═O)OH. In some embodiments, Rⁿ⁸ is hydrogen, kx8 is 1, mx8 is 1, ring A8 is phenyl, and R^{X8} is —OCH₂C(═O)OH.

In some embodiments of Formula (I), (I-1), (I-2), (I-3), (I-4), (I-5), (III-1), (III-2), (IV-1), or (IV-2), X9 is an N-methyl aromatic amino acid, optionally a bicyclic aromatic amino acid. In some embodiments, X9 is N-methyl amino acid comprising a phenyl or monocyclic heteroaryl, each of which is optionally substituted. In some embodiments, X9 is N-methyl amino acid comprising a naphthalyl or bicyclic heteroaryl, each of which is optionally substituted. In some embodiments, the bicyclic heteroaryl is a 5-6, 6-6, or 6-5 fused heteroaryl. In some embodiments, the heteroaryl comprise 1-3 ring nitrogen atoms. In some embodiments, the heteroaryl comprise 1-2 ring nitrogen atoms. In some embodiments, the heteroaryl comprise 1 ring nitrogen atom. In some embodiments, the N-methyl aromatic amino acid is N-methyl monocyclic aromatic amino acid comprising a phenyl or pyridinyl optionally substituted with one or more substituents each independently selected from halogen, —C₁₋₃alkyl, and trifluoromethyl. In some embodiments, the N-methyl aromatic amino acid is N-methyl bicyclic aromatic amino acid comprising a naphthalyl, quinolyl, or indazolyl optionally substituted with one or more substituents each independently selected from H or C₁₋₃alkyl.

In some embodiments of Formula (I), (I-1), (I-2), (I-3), (I-4), (I-5), (III-1), (III-2), (IV-1), or (IV-2), X9 is MeNal2, MeNal27N, MeF34diox, MeF34dOMe, MeF4T, MeY, MeWIMe, MeW7N, MeF3C4Me, or MeF3Me4C. In some embodiments of Formula (I), (I-1), (I-2), (I-3), (I-4), (I-5), (III-1), (III-2), (IV-1), or (IV-2), X9 is MeNal2, MeNal27N, MeF34diox, MeF34dOMe, MeF4T, MeY, MeWIMe, MeW7N, MeF3C4Me, or MeF3Me4C, each of which is optionally substituted. In some embodiments, X9 is optionally substituted MeNal2. In some embodiments. X9 is optionally substituted MeNal27N. In some embodiments, X9 is optionally substituted MeF34diox. In some embodiments, X9 is optionally substituted MeF34dOMe. In some embodiments, X9 is optionally substituted MeF4T. In some embodiments, X9 is optionally substituted MeY. In some embodiments, X9 is optionally substituted MeWIMe. In some embodiments, X9 is optionally substituted MeW7N. In some embodiments, X9 is optionally substituted MeF3C4Me. In some embodiments, X9 is optionally substituted MeF3Me4C.

In some embodiments of a peptide of Formula (I), (I-1), (I-2), (I-3), (I-4), (I-5), (III-1), (III-2), (IV-1), or (IV-2), X9 is a D-amino acid. In some embodiments of a peptide of Formula (I), (I-1), (I-2), (I-3), (I-4), (I-5), (III-1), (III-2), (IV-1), or (IV-2). X9 is an L-amino acid. In some embodiments, X9 is an N-alkylated amino acid. In some embodiments, X9 is an N-methylated amino acid. In some embodiments, X9 comprises a cyclic group. In some embodiments, X9 comprises a 5-6 membered heterocycloalkyl group. In some embodiments, X9 comprises a 5-6 membered heteroaryl group. In some embodiments, X9 comprises a bicyclic heteroaryl group. In some embodiments, X9 comprises a phenyl group. In some embodiments, X9 is a peptoid.

In some embodiments of a peptide of Formula (I), (I-1), (I-2), (I-3), (I-4), (I-5), (II-1), (III-2), (IV-1), or (IV-2), X9 has a structure of

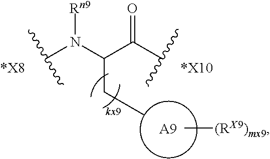

wherein:
Rⁿ⁹ is hydrogen or C₁₋₃ alkyl;
ring A9 is an aryl or heteroaryl;
each R^{X9} is independently C₁₋₆alkyl, C₁₋₆haloalkyl, C₁₋₆hydroxyalkyl, C₁₋₆aminoalkyl, C₁₋₆heteroalkyl, C₂₋₆alkenyl, C₂₋₆alkynyl, halogen, —CN, —NO₂, —OR^{a}, —SR^{a}, —SF₃, or —NR^{c}R^{d}, wherein each of the alkyl, heteroalkyl, alkenyl, and alkynyl is optionally substituted with one or more R^{X9a}; or
two R^{X9} are taken together to form ═O, ═S, or ═N(R^{a});
kx9 is 0, 1, 2, or 3;
mx9 is 0, 1, 2, 3, 4, or 5;
*X8 represents the point of attachment to X8;
*X10 represents the point of attachment to X10; and
other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (III-1), (III-2), (IV-1), or (IV-2), wherein
Rⁿ⁹ is hydrogen or methyl;
ring A9 is a C₆₋₁₀aryl or 5-10 membered heteroaryl; and
each R^{X9} is independently halogen, C₁₋₆alkyl. C₁₋₆haloalkyl, C₁₋₆hydroxyalkyl, C₁₋₆aminoalkyl, C₁₋₆heteroalkyl, C₂₋₆alkenyl, C₂₋₆alkynyl, —CN, —NO₂, —OR^{a}, —SR^{a}, —SF^{a}, or —NR^{c}R^{d}, wherein each of the alkyl, heteroalkyl, alkenyl, and alkynyl is optionally substituted with one or more R^{X9a}; or
two R^{X9} are taken together to form ═O.

In some embodiments of a peptide of Formula (I), (I-1), (I-2), (I-3), (I-4), (I-5), (III-1), (III-2), (IV-1), or (IV-2), wherein
ring A9 is a phenyl, naphthyl, pyridinyl, indolyl, azaindolyl, indazolyl, benzimidazolyl, or isoquinolinyl;
each R^{X9} is independently halogen, C₁₋₆alkyl, C₁₋₆haloalkyl, —CN, —OR^{a}, —SR^{a}, or —NR^{c}R^{d}, wherein each of the alkyl and heteroalkyl is optionally substituted with one or more R^{X9a};
kx9 is 1 or 2; and
mx9 is 0, 1, or 2.

In some embodiments, Rⁿ⁹ is hydrogen. In some embodiments, Rⁿ⁹ is C₁₋₃ alkyl. In some embodiments, Rⁿ⁹ is methyl.

In some embodiments, ring A9 is C₆₋₁₀aryl. In some embodiments, ring A9 is 5-10 membered heteroaryl. In some embodiments, ring A9 is monocyclic heteroaryl. In some embodiments, ring A9 is bicyclic heteroaryl. In some embodiments, ring A9 is bicyclic 6-6 fused heteroaryl. In some embodiments, ring A9 is bicyclic 6-5 or 5-6 fused heteroaryl. In some embodiments, ring A9 is phenyl, naphthyl, pyridinyl, indolyl, azaindolyl, indazolyl, benzimidazolyl, or isoquinolinyl.

In some embodiments, each R^{X9} is independently halogen, C₁₋₆alkyl, C₁-C₆haloalkyl, C₁-C₆hydroxyalkyl, C₁-C₆aminoalkyl, C₁-C₆heteroalkyl, —CN, —NO₂, —OR^{a}, —SR^{c}, —NR^{c}R^{d}, —S(═O)R^{a}, —S(═O)₂R^{a}, —S(═O)₂NR^{c}R^{d}, —NR^{a}S(═O)R², —NR^{a}C(═NH)(NR^{a})₂, —C(═O)R^{a}, —C(═O)OR^{a}, —OC(═O)R^{a}, —OC(═O)OR^{a}, —OC(═O)NR^{c}R^{a}, —NR^{a}C(═O)R^{a}, —NR³C(═O)OR^{a}, —NR^{a}C(═O)NR^{c}R^{d}, —C(═O)NR^{c}R^{d}, or ═O, wherein each of the alkyl and heteroalkyl is optionally substituted with one or more R^{e}. In some embodiments, each R^{X9} is independently halogen, C₁₋₆alkyl, C₁₋₆haloalkyl, —CN, —OR^{a}, —SR^{a}, or —NR^{c}R^{d}, wherein each of the alkyl and heteroalkyl is optionally substituted with one or more R^{X9a}.

In some embodiments, kx9 is 0. In some embodiments, kx9 is 1. In some embodiments, kx9 is 2. In some embodiments, kx9 is 3.

In some embodiments, mx9 is 0. In some embodiments, mx9 is 1. In some embodiments, mx9 is 2. In some embodiments, mx9 is 3. In some embodiments, mx9 is 4. In some embodiments, mx9 is 5.

In some embodiments, Rⁿ⁹ is methyl, kx9 is 1, ring A9 is a bicyclic 6-6 fused heteroaryl. In some embodiments, ring A9 is isoquinolinyl.

In some embodiments of Formula (I), (I-1), (I-2), (I-3), (I-4), (I-5), (III-1), (III-2), (IV-1), or (IV-2), X10 is G. In some embodiments, X10 is a D-amino acid (e.g., da, ds, de, or dp). In some embodiments, X10 is a D amino acid selected from da, ds, de, and dp. In some embodiments, X10 is da.

In some embodiments of Formula (I), (I-1), (I-2), (I-3), (I-4), (I-5), (III-1), (III-2), (IV-1), or (IV-2), X11 is F or a variant thereof, or an amino acid comprising —C₁₋₆alkylene-phenyl. In some embodiments, the F or the variant thereof is F, or a variant thereof comprising a phenyl or heteroaryl (e.g., monocyclic or bicyclic), each of which is optionally substituted. In some embodiments, the F or the variant thereof is F, or a variant thereof comprising a phenyl, or pyridinyl, optionally substituted with one or more substituents each independently selected from phenyl, —O-phenyl, —O—C₁₋₃alkylene-phenyl, pyridinyl, imidazolyl, pyrazolyl, Nv-C₁₋₃alkylene pyrazolyl, N—C₁₋₃alkylene(—O—C₁₋₃alkyl) pyrazolyl, pyranyl, tetrahydropyranyl, piperidinyl, N—C₁₋₃alkylene-C(═O)-piperidinyl.

In some embodiments of Formula (I), (I-1), (I-2), (I-3), (I-4), (I-5), (III-1), (III-2), (IV-1), or (IV-2), X11 is Bph, 3Py6Ph, F41Me4Pyz, F43Pyz, F44Pyz, F41Pyz, F41Me3Pyz, F41Et4Pyz, F41MeOe4Pyz, F41MeOp4Pyz, F44thp, F4Ac4pip, PhNva. PhNle, Yph, Ybn. F4tb, F4oPr, or F4CONdMe. In some embodiments of Formula (I), (I-1), (I-2), (I-3), (I-4), (I-5), (III-1), (III-2), (IV-1), or (IV-2), X11 is Bph, 3Py6Ph. F41Me4Pyz, F43Pyz, F44Pyz, F41Pyz, F41Me3Pyz, F41Et4Pyz, F41MeOe4Pyz. F41MeOp4Pyz, F44thp, F4Ac4pip, PhNva, PhlNle, Yph, Ybn, F4tb, F4oPr, or F4CONdMe, each of which is optionally substituted. In some embodiments, X11 is optionally substituted Bph. In some embodiments, X11 is optionally substituted 3Py6Ph. In some embodiments, X11 is optionally substituted F41Me4Pyz. In some embodiments, X11 is optionally substituted F43Pyz. In some embodiments, X11 is optionally substituted F44Pyz. In some embodiments, X11 is optionally substituted F41Pyz. In some embodiments, X11 is optionally substituted F41Me3Pyz. In some embodiments, X11 is optionally substituted F41Et4Pyz. In some embodiments, X11 is optionally substituted F41MeOe4Pyz. In some embodiments. X11 is optionally substituted F41MeOp4Pyz. In some embodiments, X11 is optionally substituted F44thp. In some embodiments, X11 is optionally substituted F4Ac4pip. In some embodiments, X11 is optionally substituted PhNva. In some embodiments, X11 is optionally substituted PhNle. In some embodiments, X11 is optionally substituted Yph. In some embodiments, X11 is optionally substituted Ybn. In some embodiments, X11 is optionally substituted F4tb. In some embodiments, X11 is optionally substituted F4oPr. In some embodiments, X11 is optionally substituted F4CONdMe.

In some embodiments of a peptide of Formula (I), (I-1), (I-2), (I-3), (I-4), (I-5), (II-11), (III-2), (IV-1), or (TV-2). X11 is any amino acid. In some embodiments, X11 is a D-amino acid. In some embodiments, X11 is an L-amino acid. In some embodiments, X11 is an N-alkylated amino acid. In some embodiments, X11 is an N-methylated amino acid. In some embodiments, X11 comprises a cyclic group. In some embodiments, X11 comprises a 5-6 membered heterocycloalkyl group. In some embodiments, X11 comprises a 5-6 membered heteroaryl group. In some embodiments, X11 comprises a bicyclic heteroaryl group. In some embodiments, X11 comprises a phenyl group. In some embodiments, X11 is a peptoid.

In some embodiments of a peptide of Formula (I), (I-1), (I-2), (I-3), (I-4), (I-5), (III-1), (III-2), (TV-1), or (IV-2), X11 has a structure of

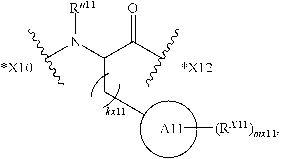

wherein:
Rⁿ¹¹ is hydrogen or C₁₋₃ alkyl;
ring A11 is an aryl or heteroaryl;
each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —SF₅, —NR^{c}R^{d}, —S(═O)R^{a}, —S(═O)R^{a}, —S(═O)₂R^{c}R^{d}, —S(═O)(═NR^{a})R^{a}, —N═S(═O)R^{c}R^{d}, —NR^{a}S(═O)₂R^{a}, amidinyl, —NR²C(═NH)NR^{c}R^{d}, —NR^{a}S(═O)₂R^{c}R^{d}, —C(═O)R², —C(═O)OR⁹, —OC(═O)R^{a}, —OC(═O)OR^{a}, —OC(═O)NR^{c}R^{c}, —NR⁹C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, aryl, heteroaryl, cycloalkyl, heterocycloalkyl, -L^{X11}-heterocycloalkyl, -L^{X11}-cycloalkyl, -L^{X11}-aryl, or -L^{X11}-heteroaryl, wherein each of the alkyl, heteroalkyl, aryl, heteroaryl, cycloalkyl, and heterocycloalkyl is optionally substituted with one or more R^{X11a}; or
two R^{X11} are taken together to form ═O, ═S, or ═N(R^{a});
L^{X11} is C₁₋₆alkylene, C₁₋₆heteroalkylene, —O—, —S—, or —NR^{a}—, wherein the alkylene and heteroalkylene is optionally substituted with one or more R^{X11a};
kx11 is 0, 1, 2, 3, 4, or 5;
mx11 is 0, 1, 2, 3, 4, or 5;
*X10 represents the point of attachment to X10;
*X12 represents the point of attachment to X12 or *X12 represents the point of attachment to N(R²) (e.g., in Formula (I-1) and (I-2)); and
other groups (such as R^{a}, R^{c}, R^{d}, and R^{e}) have the meanings defined in Formula (I).

In some embodiments of a peptide of Formula (I), (I-1), (I-2), (I-3), (I-4), (I-5), (II-1), (III-2), (IV-1), or (IV-2), wherein
ring A11 is C₁₋₆aryl or 5-10 membered heteroaryl;
each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —SF₅, —NR^{c}R^{d}, —S(═O)R^{a}, —S(═O)₂R, —S(═O)₂R^{c}R^{d}, —S(═O)(═N a)R^{a}, —N═S(═O)R^{c}R^{d}, —NR^{a}S(═O)₂R^{a}, amidinyl, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, —P(═O)(OR^{c})(OR^{d}), —P(═O)R^{c}R^{d}, C₆₋₁₀aryl, 5-10 membered heteroaryl, C₃₋₆cycloalkyl, 5-6 membered heterocycloalkyl, -L^{X11}-5-6 membered heterocycloalkyl, -L^{X11}-C₃₋₆cycloalkyl, -L^{X11}-C₆₋₁₀aryl, or -L^{X11}-5-10 membered heteroaryl, wherein each of the alkyl, heteroalkyl, aryl, heteroaryl, cycloalkyl, and heterocycloalkyl is optionally substituted with one or more R^{X11a}; or
two R^{X11} are taken together to form ═O and
L^{X11} is C₁₋₆alkylene, C₁₋₆heteroalkylene, or —O—, wherein the alkylene and heteroalkylene is optionally substituted with one or more R^{X11a}.

In some embodiments of a peptide of Formula (I), (I-1), (I-2), (I-3), (I-4), (I-5), (III-1), (III-2), (IV-1), or (IV-2), wherein
ring A11 is a phenyl or pyridinyl;
each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —NR^{c}R^{d}, —S(═O)R^{a}, —S(═O)₂R^{a}, —S(═O)₂R^{c}R^{d}, —NR^{a}S(═O)R^{a}, —NR^{a}C(═NH)NR^{c}R^{d}, —NR^{c}S(═O)₂R^{c}R^{d}, —C(═O)R^{a}, —C(═O)OR^{c}, —OC(═O)R^{a}, —OC(═O)OR^{a}, —OC(═O)NR^{c}R^{d}, —NR^{a}C(═O)R^{a}, —NR^{a}C(═O)OR^{a}, —NR^{a}C(═O)NR^{c}R^{d}, —C(═O)NR^{c}R^{d}, C₆₋₁₀aryl, 5-10 membered heteroaryl, C₃₋₆cycloalkyl, 5-6 membered heterocycloalkyl, -L^{X11}5-6 membered heterocycloalkyl, -L^{X11}-C₃₋₆cycloalkyl, -L^{X11}-C₆₋₁₀aryl, or -L^{X11}-5-10 membered heteroaryl, wherein each of the alkyl, heteroalkyl, aryl, heteroaryl, cycloalkyl, and heterocycloalkyl is optionally substituted with one or more R^{X11a}; or
two R^{X11} are taken together to form ═O;
kx11 is 1, 2, 3, or 4; and
mx11 is 0, 1, or 2.

In some embodiments, Rⁿ¹ is hydrogen. In some embodiments, Rⁿ¹¹ is C₁₋₃ alkyl. In some embodiments, Rⁿ¹¹ is methyl.

In some embodiments. A11 is C₆₋₁₀aryl. In some embodiments, A11 is phenyl. In some embodiments, A11 is 5-10 membered heteroaryl. In some embodiments, A11 is 5-6 membered heteroaryl. In some embodiments, A11 is monocyclic heteroaryl. In some embodiments, A11 is bicyclic heteroaryl.

In some embodiments, each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —SF₅, —NR^{c}R^{d}, —S(═O)R^{a}, —S(═O)₂R², —S(═O)₂R^{c}R^{d}, —S(═O)(═NR^{a})R^{a}, —N═S(═O)R^{c}R^{d}, —NR^{a}S(═O)₂R^{a}, amidinyl, —NR^{c}C(═NH)NR^{c}R^{d}, —NR^{a}S(═O)₂R^{c}R^{d}, —C(═O)R^{a}, —C(═O)OR², —OC(═O)R^{a}, —OC(═O)OR^{a}, —OC(═O)NR^{c}R^{d}, —NR^{a}C(═O)R^{a}, —NR^{a}C(═O)ORₐ, —NR^{a}C(═O)NR^{c}R^{d}, —C(═O)NR^{c}R^{d}, —P(═O)(OR^{c})(OR^{a}), —P(═O)R^{c}R^{d}, C₆₋₁₀aryl, 5-10 membered heteroaryl, C₃₋₆cycloalkyl, 5-6 membered heterocycloalkyl, -L^{X11}-5-6 membered heterocycloalkyl, -L^{X11}-C₃₋₆cycloalkyl, -L^{X11}-C₆₋₁₀aryl, or -L^{X11}-5-10 membered heteroaryl, wherein each of the alkyl, heteroalkyl, aryl, heteroaryl, cycloalkyl, and heterocycloalkyl is optionally substituted with one or more R^{X11a}.

In some embodiments, each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —NR^{c}R^{d}, —S(═O)R^{a}, —S(═O)₂R^{a}, —S(═O)₂R^{c}R^{d}, —NR^{a}S(═O)₂R^{a}, —NR^{a}C(═NH)NR^{c}R^{d}, —NR^{a}S(═O)₂R^{c}R^{d}, —C(═O)R^{a}, —C(═O)OR^{a}, —OC(═O)R^{a}, —OC(═O)OR^{a}, —OC(═O)NR^{c}R^{d}, —NR^{a}C(═O)R^{c}, —NR^{a}C(═O)OR^{a}, —NR^{a}C(═O)NR^{c}R^{d}, —C(═O)NR^{c}R^{d}, C₆₋₁₀aryl, 5-10 membered heteroaryl, C₃₋₆cycloalkyl, 5-6 membered heterocycloalkyl, -L^{X11}-5-6 membered heterocycloalkyl, -L^{X11}-C₃₋₆cycloalkyl, -L^{X11}-C₆₋₁₀aryl, or -L^{X11}-5-10 membered heteroaryl, wherein each of the alkyl, heteroalkyl, aryl, heteroaryl, cycloalkyl, and heterocycloalkyl is optionally substituted with one or more R^{X11a}.

In some embodiments, each R^{X11} is independently C₁₋₆alkyl, C₁₋₆haloalkyl, C₁₋₆hydroxyalkyl, C₁₋₆aminoalkyl, C₁₋₆heteroalkyl, halogen, —CN, —NO₂, —OR^{a}, —SR^{a}, —NR^{c}R^{a}, —C(═O)R², —C(═O)OR^{a}, —OC(═O)R^{a}, —NR^{a}C(═O)R^{a}, —C(═O)NR^{c}R^{d}, C₆₋₁₀aryl, 5-10 membered heteroaryl, C₃₋₆cycloalkyl, 5-6 membered heterocycloalkyl, -L^{X11}-5-6 membered heterocycloalkyl, -L^{X11}-C₃₋₆cycloalkyl, -L^{X11}-C₆₋₁₀aryl, or -L^{X11}-5-10 membered heteroaryl, wherein each of the alkyl, heteroalkyl, aryl, heteroaryl, cycloalkyl, and heterocycloalkyl is optionally substituted with one or more R^{X11a}.

In some embodiments, each R^{X11} is independently phenyl, pyridinyl, pyrrolyl, pyrazolyl, imidazolyl, cyclohexyl, piperidinyl, piperazinyl, morpholinyl, tetrahydropyranyl, -L^{X1}5-6 membered heterocycloalkyl, -L^{X11}-phenyl, or -L^{X11}-pyridinyl, wherein each of the phenyl, pyridinyl, pyrrolyl, pyrazolyl, imidazolyl, cyclohexyl, piperidinyl, piperazinyl, morpholinyl, tetrahydropyranyl, and heterocycloalkyl is optionally substituted with one or more R^{X11a}.

In some embodiments, two R^{X11} are taken together to form oxo.

In some embodiments, L^{X11} is C₁₋₆alkylene, which is optionally substituted with one or more R^{X11a}. In some embodiments, L^{X11} is C₁₋₆heteroalkylene, which is optionally substituted with one or more R^{X11a}. In some embodiments, L^{X11} is —O—. In some embodiments, L^{X11} is —S—. In some embodiments, L^{X11} is —NR^{a}—.

In some embodiments, each R^{X11a} is independently halogen, C₁₋₆alkyl, C₁-C₆haloalkyl, C₁-C₆hydroxyalkyl, C₁-C₆aminoalkyl, C₁-C₆heteroalkyl, —CN, —NO₂, —OR^{a}, —SR², —NR^{c}R^{d}, —S(═O)R^{a}, —S(═O)₂R^{a}, —S(═O)₂NR^{c}R^{d}, —NR^{a}S(═O)₂R^{a}, —NR^{a}C(═NH)(NR)₂, —C(═O)R^{a}, —C(═O)OR^{a}, —OC(═O)R^{a}, —OC(═O)OR^{a}, —OC(═O)NR^{c}R^{d}, —NR^{a}C(═O)R^{a}, —NR^{a}C(═O)OR², —NR²C(═O)NR^{c}R^{d}, —C(═O)NR^{c}R^{d}, or ═O, wherein each of the alkyl and heteroalkyl is optionally substituted with one or more R^{e}.

In some embodiments, kx11 is 0. In some embodiments, kx11 is 1. In some embodiments, kx11 is 2. In some embodiments, kx11 is 3. In some embodiments, kx11 is 4. In some embodiments, kx11 is 5.

In some embodiments, mx11 is 0. In some embodiments, mx11 is 1, In some embodiments, mx11 is 2. In some embodiments, mx11 is 3. In some embodiments, mx11 is 4. In some embodiments, mx11 is 5.

In some embodiments, Rⁿ¹¹ is hydrogen, kx11 is 0, 1, or 2, mx11 is 1, ring A11 is monocyclic heteroaryl, and R^{X11} is phenyl. In some embodiments, Rⁿ¹¹ is hydrogen, kx11 is 1, mx11 is 1, ring A11 is pyridinyl, and R^{X11} is phenyl.

In some embodiments of Formula (I), (I-1), (I-2), (I-3), (I-4), (I-5), (III-1), (III-2), (IV-1), or (IV-2), X12 is N-methylated cysteine or a derivative thereof. In some embodiments, X12 is N-methylated cysteine. In some embodiments, X12 is N-methylated cysteine, which is optionally substituted.

In some embodiments of Formula (I), (I-1), (I-2), (I-3), (I-4), (I-5), (III-1), (III-2), (IV-1), or (IV-2),
X1 is I, R, Cit, F40, 4Py, 3Py, KCOpipzaa, V, Eva, Q, E, MeI, Ahp, F4COO, KCOpip4COO, MeQdMe, MeA, MeSMe, MeG, MeV, MeHseMe, Aib, MeT, alI, TMe, MeKCOpipzaa, MeQ, Hpr, MeTMe, MeDapCOpipzaa; MeK, MeKAc, MeK(de), MeK(I), MeK(df), or MeK(datb);
X2 is I, K, Cit. F4G, 4Py, 3PV, KCOpipzetOH, V. KCOpipzaa, Ev a, Q, E, S, Ahp, F4COO, KCOpip4COO, MeQdMe, MeA, MeSMe, MeG, MeI, MeV, MeL, HseMe, MeY, Me3Py, MeHseMe, MeKAc, alI, TMe MeKCOpipzaa, MeQ, Hpr, MeTMe, or MeDapCOpipzaaa;
X3 is D, Har, KCOpipzetOH, Cit, KCOmeglumine, KCOpipzaa, A4paa, Q, A, E, MeD, S, N. Hgl, F4COO, KCOpip4COO, KAc, Hgn, MeY, or DapCOpipzaa;
X4 is D, Har, KCOpipzetOH, KCOmegiumine, KCOpipzaa, A4paa, Q, A, E, MeD, S, N, Hgl, F4COO, KCOpip4COO, dd, MeQ, MeQdMe, MeA, MeSMe, MeG, EtG, MeeG, CmG, CmpG, CrmG, CeG, CrpG, MeK, MeKAc, MeHgl, Hgn, MeDapCOpipzaa, MeKCOpipzaa, Medd, Cit, MeCit, MeN, MeS, MeE, MeY, W5N, or MeA4paa;
X5 is Y, F3G, 3Py6COO,4Pv2NH2, 3Py5COO, F3COO, 3Pv6NHAc, F. F4C. F40Me, F4COO, Nal2, F3aao, F4aa, F4aao, 3Py6NHaa, 5Pdo, F3CON, F4F, F40Et, F4Me, F4CON, F4CONPEG4Me, F3OMe, F3CON, Yae, YaeCOpipzaa, F4aaopipzaa, 4Pdo, 3Py6CON, Atp, Cha4cH, Cha4tH, Cha4cOMe, A1mor, or F4amCOpipzaa;
X6 is I, V, Eva, Chg, Tbg, A, L, Ahp, F4COO, Gcpr, Gcpe, alI, Cle, S3REt, TMe, Acpr, Cba. Gthp, NleCOO, NIeOH, P, Atb. Nva, Nle, N, DapAc, Abu, Nmm, Ndm, Ncit. Cit, SMe, HseMe, HseEt, HseiPr, dMeS, TdMe, Cbg, NvaOMe, SiPr, Spr, NIeOMe, Sbu, Sebm, Sepe, AhpOMe, HseBu, Spent or Hsecpe;
X7 is I, Eva, alI, TMe, SMe, Gepr, Gepe, Gthp, dMeS, TdMe, or Cbg;
X8 is A, I, L, V, Y, F40Me, F4COO, F40Et, F4u, F4Me, F4CONdMe, F4CON, F4 ms, F4CONPEG4Me, F34dOMe, F30Me, F3C, F3CON, F3CONdMe, 3Py6CON, Yae, YaeCOpipzaa, 51nda, F3aao, F3aa, F4aao, F4aa, 3Py6Nhae, 3Py6NHAc, 3Py6OMe, F4aaopipzaa, or F4amCOpipzaa;
X9 is MeNal2, MeNal27N, MeF34diox, MeF34dOMe, MeF4T, MeY, MeWIMe, MeW7N, MeF3C₄Me, or MeF3Me4C;
X10 is G or a D-amino acid (e.g., da, ds, de, or dp);
X11 is Bph, 3Py6Ph, F41Me4Pyz, F43Pyz, F44Pyz, F41Pyz, F41Me3Pyz, F41Et4Pyz, F41MeOe4Py z, F41MeOp4Pyz. F44thp. F4Ac4pip, PhNva, PhNle, Yph, Ybn, F4tb, F4oPr, or F4CONdMe; and
X12 is MeC.

In some embodiments of Formula (I), (I-5), (III-2), or (IV-2),
X1 is MeA, MeK, MeK(datb), MeK(de), MeK(df), MeK(H), MeKAc, MeKCOpipzaa, MeDapCOpipzaa. or MeQ;
X2 is MeA, HseMe, MeHseMe, MeI, MeL, MeQ, MeY, S, TMe, MeKCOpipzaa, or Me3Py;
X3 is D, Hgn, KAc, MeY, or Q;
X4 is MeA4paa, MeD. MeK, Q, MeQ. MeY, W5N, MeDapCOpipzaa, or MeKCOpipzaa;
X5 is 3Py6NHaa, F4aao, F4COO, Y, or Yae;
X6 is alI, Cle, 1, S3REt, or TMe;
X7 is I;
X8 is F4aa, F4aao, F4aaopipzaa, F4amCOpipzaa. F4COO, F4u, Y, Yae, or YaeCOpipzaa;
X9 is MeNa12, MeNa127N, or MeY;
X10 is G;
X11 is 3Py6Ph or F41Me4Pyz; and
X12 is MeC.

In some embodiments of Formula (I), (I-1), (I-2), (I-3), (I-4), (I-5), (III-1), (III-2), (IV-1), or (IV-21),
X1 is MeA, MeK, MeK(datb), MeK(de), MeK(df), MeK(H), MeKAc, MeKCOpipzaa, MeDapCOpipzaa, or MeQ;
X2 is MeA, HseMe, MeHseMe. MeI, MeL, MeQ, MeY, S, TMe, MeKCOpipzaa, or Me3Py;
X3 is D, Hgn, KAc, MeY, or Q;
X4 is MeA4paa, MeD, MeK, Q, MeQ, MeY, W5N, MeDapCOpipzaa, or MeKCOpipzaa;
X5 is 3Py6NHaa, F4aao, F4COO, Y, or Yae;
X6 is alI, Cle, I, S3REt, or TMe;
X7 is 1;
X8 is F4aa, F4aao, F4aaopipzaa, F4amCOpipzaa, F4COO, F4u, Y, Yae, or YaeCOpipzaa;
X9 is MeNaI2, MeNal27N, or MeY;
X10 is G; and
X11 is 3Py6Ph or F41Me4Pyz.

In some embodiments, a radiopharmaceutical conjugate of the present disclosure comprising a cyclic peptide of Formula (I) has a structure of Formula (III-1),

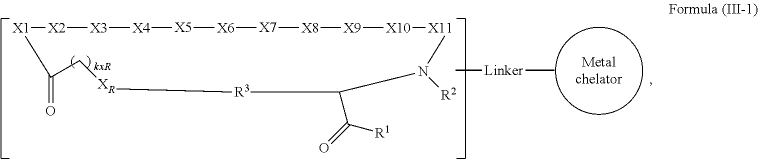

wherein,
   -Linker- represents the linker;
   R¹ is selected from the group consisting of —NH₂ and —OH;
   R² is C₁₋₃ alkyl;
   R³ is C₁₋₃ alkylene, optionally substituted with one or more R⁴;
   each R⁴ is independently C₁₋₃ alkyl or C₃₋₆ cycloalkyl;
   kxR is 1, 2, 3, 4, 5, or 6;
   X_{R} is selected from the group consisting of —S—, —CH₂—, or —O—; and
wherein X1 to X11 have the definitions described in Formula (I).

In some embodiments, the linker is attached to the 1^{st} amino acid residue (or X1), the 2^{nd} amino acid residue (or X2), the 3^{rd} amino acid residue (or X3), the 4^{th} amino acid residue (or X4), the 8^{th} amino acid residue (or X8), or the 12^{th} amino acid residue at R¹. In some embodiments, the linker is attached to the 1^{st} amino acid residue (or X1). In some embodiments, the linker is attached to the 2^{nd} amino acid residue (or X2). In some embodiments, the linker is attached to the 3^{rd} amino acid residue (or X3). In some embodiments, the linker is attached to the 3^{rd} amino acid residue (or X3). In some embodiments, the linker is attached to the 4^{th} amino acid residue (or X4). In some embodiments, the linker is attached to the 8^{th} amino acid residue (or X8). In some embodiments, the linker is attached to the 12^{th} amino acid residue at R¹. In some embodiments, the linker is a bond. In some embodiments, the metal chelator is

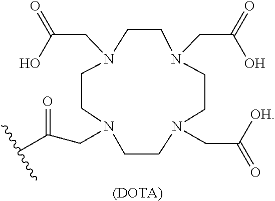

In some embodiments, the radiopharmaceutical conjugate further comprises a radionuclide bound to the metal chelator.

In some embodiments, a radiopharmaceutical conjugate of the present disclosure comprising a cyclic peptide of Formula (I) has a structure of Formula (III-2),

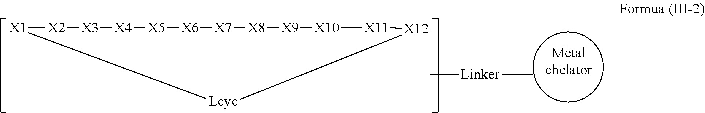

wherein
   X1-X12 have the definition described in Formula (I) and Lcyc is a ring closing group that covalently connecting X1 with X12; and
   -Linker- represents the linker.

In some embodiments, the linker is attached to the 1^{st} amino acid residue (or X1), the 2^{nd} amino acid residue (or X2), the 3^{rd} amino acid residue (or X3), the 4^{th} amino acid residue (or X4), the 8^{th} amino acid residue (or X8), or the 12^{th} amino acid residue (or X12). In some embodiments, the linker is attached to the 1^{st} amino acid residue (or X1). In some embodiments, the linker is attached to the 2^{nd} amino acid residue (or X2). In some embodiments, the linker is attached to the 3^{rd} amino acid residue (or X3). In some embodiments, the linker is attached to the 3^{rd} amino acid residue (or X3). In some embodiments, the linker is attached to the 4^{th} amino acid residue (or X4). In some embodiments, the linker is attached to the 8^{th} amino acid residue (or X8). In some embodiments, the linker is attached to the 12^{th} amino acid residue (or X12). In some embodiments, the linker is a bond. In some embodiments, the metal chelator is

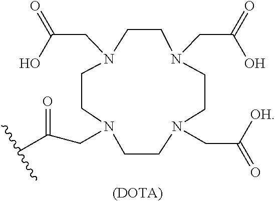

In some embodiments, the radiopharmaceutical conjugate further comprises a radionuclide bound to the metal chelator.

In some embodiments, a radiopharmaceutical conjugate of the present disclosure comprising a cyclic peptide of Formula (I) has a structure of Formula (IV-1),

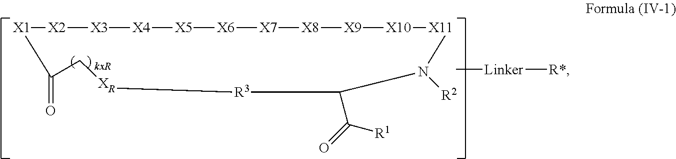

wherein,
   -Linker- represents the linker;
   R* is a covalently bound radionuclide;
   R¹ is selected from the group consisting of —NH₂ and —OH;
   R² is C₁₋₃ alkyl;
   R³ is C₁₋₃ alkylene, optionally substituted with one or more R⁴;
   each R⁴ is independently C₁₋₃ alkyl or C₃₋₆ cycloalkyl;
   kxR is 1, 2, 3, 4, 5, or 6;
   X_{R} is selected from the group consisting of —S—, —CH₂—, or —O—; and
wherein X1 to X11 have the definitions described in Formula (I).

In some embodiments, the linker is attached to the 5^{th} amino acid residue (or X5), the 8^{th} amino acid residue (or X8), the 9^{th} amino acid residue (or X9), or the 11^{th} amino acid residue (or X11). In some embodiments, the linker is attached to the 5^{th} amino acid residue (or X5). In some embodiments, the linker is attached to the 8^{th} amino acid residue (or X8). In some embodiments, the linker is attached to the 9^{th} amino acid residue (or X9). In some embodiments, the linker is attached to the 11^{th} amino acid residue (or X11). In some embodiments, the linker is a bond.

In some embodiments, a radiopharmaceutical conjugate of the present disclosure comprising a cyclic peptide of Formula (I) has a structure of Formula (IV-2).

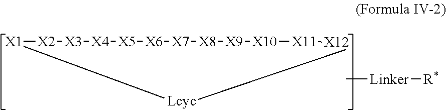

wherein
   X1-X12 have the definition described in Formula (I) and Lcyc is a ring closing group that covalently connecting X1 with X12; and
   -Linker- represents the linker.

In some embodiments, the linker is attached to the 5^{th} amino acid residue (or X5), the 8^{th} amino acid residue (or X8), the 9^{th} amino acid residue (or X9), or the 11^{th} amino acid residue (or X11). In some embodiments, the linker is attached to the 5^{th} amino acid residue (or X5). In some embodiments, the linker is attached to the 8^{th} amino acid residue (or X8). In some embodiments, the linker is attached to the 9^{th} amino acid residue (or X9). In some embodiments, the linker is attached to the 11^{th} amino acid residue (or X11). In some embodiments, the linker is a bond.

In some embodiments, the Lcyc is a group selected from Table 4B. In some embodiments, the Lcyc is formed by reacting the first and the second functional groups in Table 4C. In some embodiments, the Lcyc is —C(═O)—CH₂—. In some embodiments, the Lcyc is —C(═O)—CH—, which is formed by reacting with a chloroacetylated (or bromoacetylated) amino acid with a cysteine. In some embodiments, the Lcyc is —C(═O)—CH₂—S—, which is formed by reacting with a chloroacetylated (or bromoacetylated) amino acid with an amino acid comprising an SH group.

In some embodiments, a radiopharmaceutical conjugate disclosed herein or a pharmaceutically accepted salt thereof has a cyclic structure having an amino acid (e.g., a chloroacetylated amino acid) in the first residue X1 and a cysteine residue or a variant thereof, and wherein the amino acid (e.g., the chloroacetylated amino acid) in X1 and the cysteine residue or a variant thereof are bound. In some embodiments, a radiopharmaceutical conjugate disclosed herein or a pharmaceutically accepted salt thereof has a cyclic structure having an amino acid (e.g., a chloroacetylated amino acid) in the first residue X1 and a cysteine residue or a variant thereof, and wherein the amino acid (e.g., the chloroacetylated amino acid) in X1 and the cysteine residue or a variant thereof form a covalent bond. In some embodiments, a radiopharmaceutical conjugate disclosed herein or a pharmaceutically accepted salt thereof has a cyclic structure having a bromoacetylated amino acid in the first residue X1 and a cysteine residue or a variant thereof, and wherein the bromoacetylated amino acid in X1 and the cysteine residue or a variant thereof form a covalent bond.

In some embodiments, the radiopharmaceutical conjugate consists of an amino acid sequence selected from SEQ ID NOs: 1-72, and the peptide has a cyclic structure having a cysteine residue or a variant thereof at the 12^{th} residue (X12). In some embodiments, the peptide consists of an amino acid sequence selected from SEQ ID NOs: 1-72, and the peptide has a cyclic structure having a cysteine residue or a variant thereof at the 12^{th} residue (X12), and wherein the chloroacetylated amino acid and the cysteine residue or a variant thereof at 12^{th} residue form a covalent bond. In some embodiments, the chloroacetyl group can be replaced with a bromoacetyl group.

In some embodiments, the peptide consists of an amino acid sequence selected from any one of SEQ ID NOs: 66-71 that lacks the X3 amnio acid residue, and the peptide has a cyclic structure having a cysteine residue or a variant thereof at the “12^{th} residue” (X12). In some embodiments, the peptide consists of an amino acid sequence selected from SEQ ID NOs: 1-72, and the peptide has a cyclic structure having a cysteine residue or a variant thereof at the 12^{th} residue (X12), and wherein the amino acid at X1 (e.g., a chloroacetylated amino acid) and the cysteine residue or a variant thereof at the 12^{th} residue form a covalent bond. In some embodiments, the chloroacetyl group can be replaced with a bromoacetyl group.

In some embodiments, a radiopharmaceutical conjugate disclosed herein or a pharmaceutically salt thereof has a cyclic structure having the first amino acid covalently linked to the last amino acid.

In some embodiments, the radiopharmaceutical conjugate or the pharmaceutically accepted salt thereof has a cyclic structure having a chloroacetylated amino acid in X1 and a cysteine or a cysteine variant thereof residue, and wherein the chloroacetylated amino acid in X1 and the cysteine or substituted cysteine are bound. In some embodiments, the peptide consists of an amino acid sequence selected from SEQ ID NOs: 1-72. In some embodiments, the peptide consists of an amino acid sequence selected from SEQ ID NOs: 1-72, and the peptide has a cyclic structure. In some embodiments, the peptide consists of an amino acid sequence selected from SEQ ID NOs: 1-72, and the peptide has a cyclic structure having a chloroacetylated amino acid and a cysteine or a cysteine variant thereof residue at C-terminus, and wherein the chloroacetylated amino acid and the cysteine or a cysteine variant thereof at C-terminus are bound. In some embodiments, the peptide has a cyclic structure having a chloroacetylated amino acid and; (i) 12 or more total amino acids, a cysteine or a cysteine variant thereof residue at the 12^{th} residue, and wherein the chloroacetylated amino acid and the cysteine or a cysteine variant thereof at the 12^{th} residue are bound; or (ii) 11 or fewer total amino acids, especially lacking X3 residue, a cysteine or a cysteine variant thereof residue at the 12^{th} residue, and wherein the chloroacetylated amino acid and the cysteine or substituted cysteine at the 12^{th} residue are bound. In some embodiments, the chloroacetyl group can be replaced with a bromoacetyl group.

In some embodiments, the radiopharmaceutical conjugate or the pharmaceutically acceptable salt thereof comprises a peptide comprising an amino acid sequence that is at least 95% identical to a sequence selected from SEQ ID NOs: X1-X12 of SEQ ID NOs: 1-72. In some embodiments, the peptide or the salt thereof comprises an amino acid sequence that is at least 80%, 85%, 90%, 95%, or 98% identical to a sequence selected from SEQ ID NOs: X1-X12 of SEQ ID NOs: 1-72. In some embodiments, the peptide or the salt thereof consists of an amino acid sequence selected from SEQ ID NOs: X1-X12 of SEQ ID NOs: 1-72. In some embodiments, the peptide or the salt thereof comprises an amino acid sequence that has at most 1, 2, 3, 4, or 5 amino acid residues that are different compared to a sequence selected from SEQ ID NOs: X1-X12 of SEQ ID NOs: 1-72. In some embodiments, the peptide or the salt thereof comprises an amino acid sequence that has at most 1, 2, 3, 4, or 5 additions, deletions and/or substitutions (including conservative substitutions) to a sequence selected from SEQ ID NOs: X1-X12 of SEQ ID NOs: 1-72. In some embodiments, the peptide or the salt thereof comprises an amino acid sequence that has at most 1 addition, deletion, or substitution (including conservative substitutions) to a sequence selected from SEQ ID NOs: X1-X12 of SEQ ID NOs: 1-72.

In some embodiments, the peptide or the salt thereof comprises an amino acid sequence that lacks the X3 amino acid residue of any one of SEQ ID NOs: 1-72, and is at least 70%, 80%, or 90% identical to the remaining residues of any one of SEQ ID NOs: 1-72. In some embodiments, the peptide or the salt thereof comprises an amino acid sequence that is at least 70%, 80%, or 90% identical to a sequence selected from SEQ ID NOs: 66-71.

Exemplary peptides of the present disclosure include the peptides described in Table 1. In some embodiments, the peptides of Table 1 have a —C(═O)-halogen group attached to the N-terminus. In some embodiments, the peptides of Table 1 have a —C(═O)—CH₂-halogen group attached to the N-terminus. In some embodiments, the peptides of Table 1 have a —C(═O)-halogen group attached at residue position 1. In some embodiments, the peptides of Table 1 have a —C(═O)—CH₂-halogen group attached at residue position 1. In some embodiments, the peptides of Table 1 have a —C(═O)—Cl group attached to the N-terminus. In some embodiments, the peptides of Table 1 have a —C(═O)—CH₂—Cl group attached to the N-terminus. In some embodiments, the peptides of Table 1 have a —C(═O)—Cl group attached at residue position 1. In some embodiments, the peptides of Table 1 have a —C(═O)—CH₂—Cl group attached at residue position 1. In some embodiments, the peptides of Table 1 have a —C(═O)—Br group attached at residue position 1. In some embodiments, the peptides of Table 1 have a —C(═O)—CH₂—Br group attached at residue position 1.

In some embodiments, the radiopharmaceutical conjugates of the disclosure have a —C(═O)-halogen group attached to the N-terminus. In some embodiments, the radiopharmaceutical conjugates of the disclosure have a —C(═O)—CH₂-halogen group attached to the N-terminus. In some embodiments, the radiopharmaceutical conjugates of the disclosure have a —C(═O)-halogen group attached at residue position 1. In some embodiments, the radiopharmaceutical conjugates of the disclosure have a —C(═O)—CH₂-halogen group attached at residue position 1. In some embodiments, the radiopharmaceutical conjugates of the disclosure have a —C(═O)—Cl group attached to the N-terminus. In some embodiments, the radiopharmaceutical conjugates of the disclosure have a —C(═O)—CH₂-Cl group attached to the N-terminus. In some embodiments, the radiopharmaceutical conjugates of the disclosure have a —C(═O)—Cl group attached at residue position 1. In some embodiments, the radiopharmaceutical conjugates of the disclosure have a —C(═O)—CH₂—Cl group attached at residue position 1. In some embodiments, the radiopharmaceutical conjugates of the disclosure have a —C(═O)—Br group attached at residue position 1. In some embodiments, the radiopharmaceutical conjugates of the disclosure have a —C(═O)—CH₂—Br group attached at residue position 1. In some embodiments, the peptides in the radiopharmaceutical conjugates of the disclosure are monocyclic.

In some embodiments, the peptides of the radiopharmaceutical conjugates described herein are monocyclic peptides, wherein the —C(═O)—Cl at residue position 1 forms a bond with the cysteine at residue position 12. In some embodiments, the peptides of the radiopharmaceutical conjugates described herein are monocyclic peptides, wherein the —C(═O)—CH₂—Cl at residue position 1 forms a bond with the cysteine at residue position 12. In some embodiments, the peptides in the radiopharmaceutical conjugates described herein are monocyclic peptides with 12 amino acid residues forming the ring.

In some embodiments, a herein described conjugate is selected from conjugates described in Table 2, Table 3A or Table 3B.

In some embodiments, described herein is a conjugate having the following structure, or a pharmaceutically acceptable salt thereof:

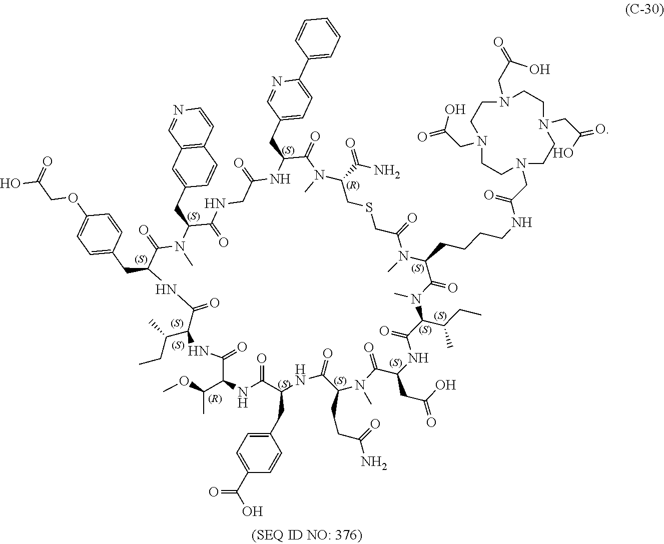

In some embodiments, described herein is a conjugate having the following structure, or a pharmaceutically acceptable salt thereof:

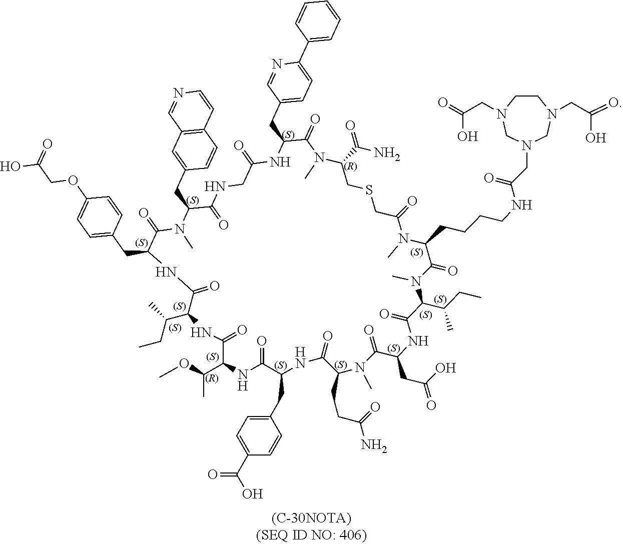

In some embodiments, described herein is a conjugate having the following structure, or a pharmaceutically acceptable salt thereof:

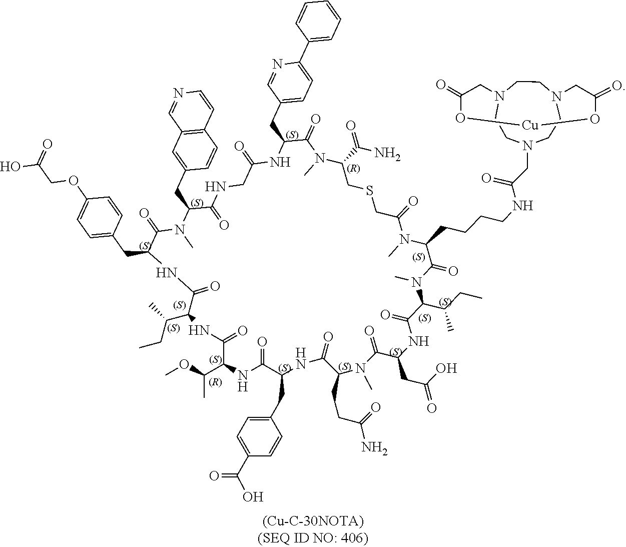

In some embodiments, Cu-C-30NOTA is ⁶⁴Cu-C-30NOTA. In some embodiments, described herein is a conjugate having the following structure, or a pharmaceutically acceptable salt thereof (SEQ ID NO: 376):

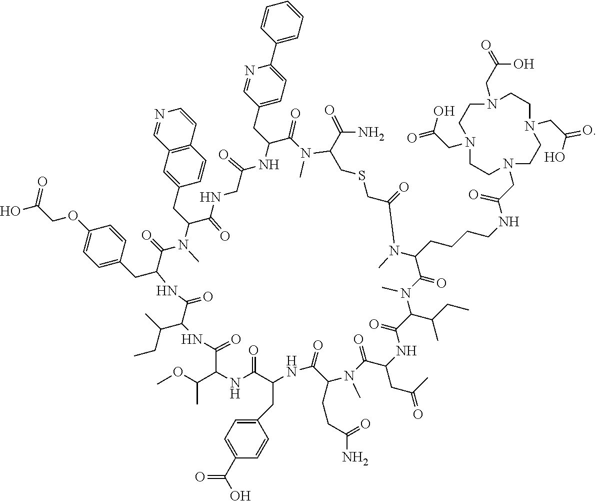 [SMILES: CCC(C)C1NC(=O)C(C(C)OC)NC(=O)C(Cc2ccc(C(=O)O)cc2)NC(=O)C(CCC(N)=O)N(C)C(=O)C(CC(C)=O)NC(=O)C(C(C)CC)N(C)C(=O)C(CCCCNC(=O)CN2CCN(CC(=O)O)CCN(CC(=O)O)CCN(CC(=O)O)CC2)N(C)C(=O)CSCC(C(N)=O)N(C)C(=O)C(Cc2ccc(-c3ccccc3)nc2)NC(=O)CNC(=O)C(Cc2ccc3ccncc3c2)N(C)C(=O)C(Cc2ccc(OCC(=O)O)cc2)NC1=O]

In some embodiments, described herein is a radiopharmaceutical having the following structure, or a pharmaceutically acceptable salt thereof (SEQ ID NO: 376):

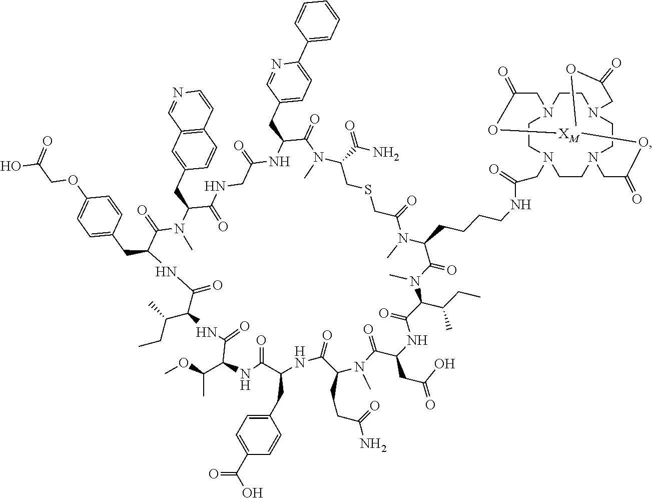

wherein X_{M} is a radionuclide. In some embodiments, described herein is a radiopharmaceutical having the following structure, or a pharmaceutically acceptable salt thereof (SEQ ID NO: 376):

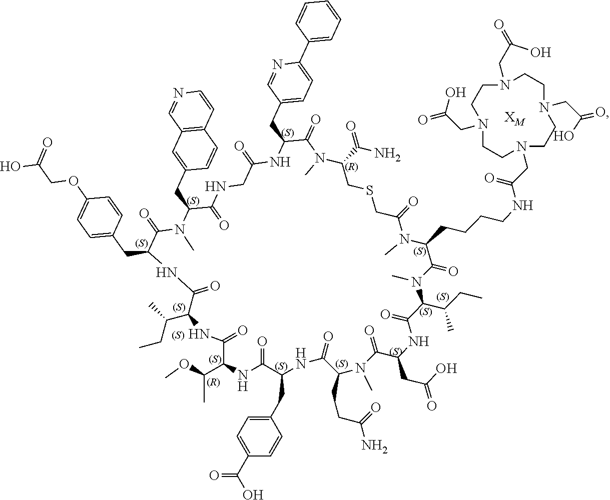

wherein X_{M} is a radionuclide. In some embodiments, described herein is a radiopharmaceutical having the following structure, or a pharmaceutically acceptable salt thereof (SEQ ID NO: 376):

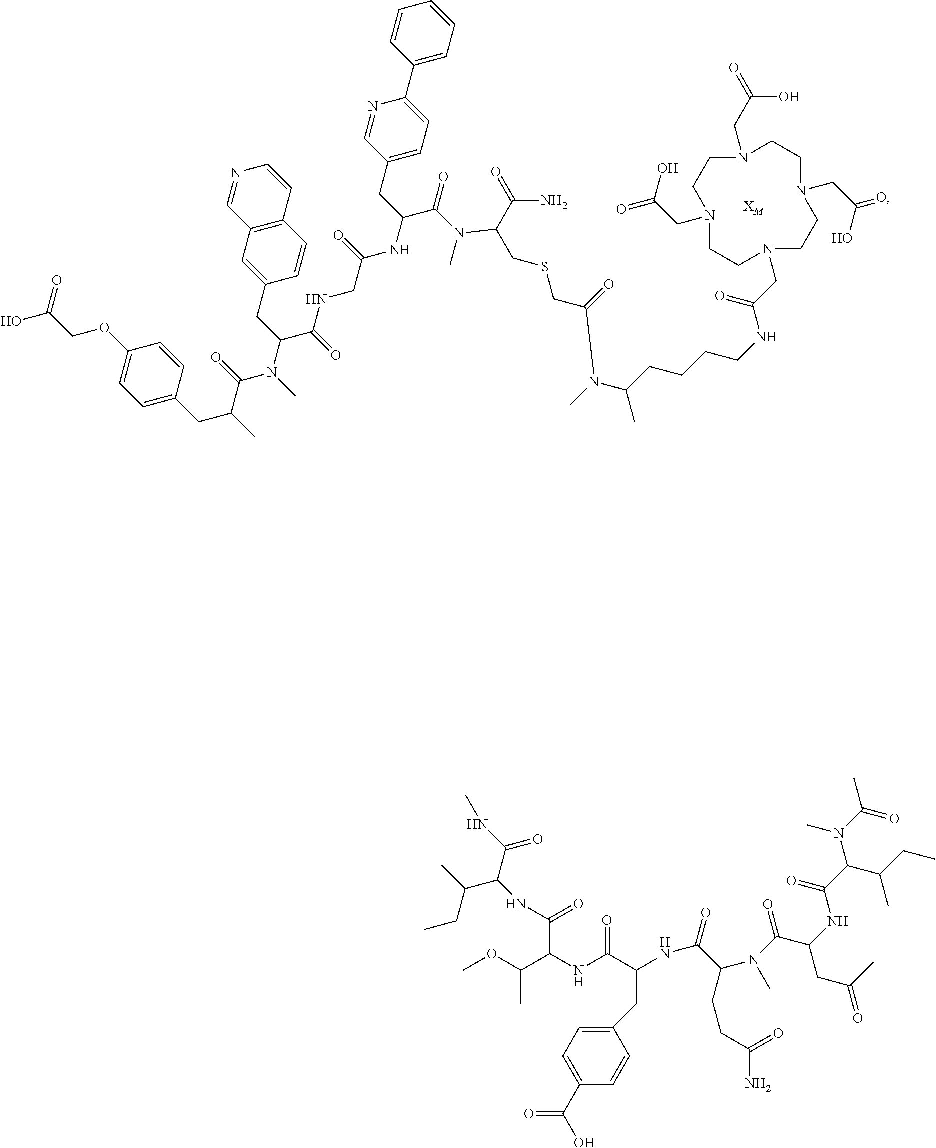

wherein X_{M} is a radionuclide. In some embodiments, X_{M} is a radionuclide selected from Table 7. In some embodiments, X_{M} is Ac-225. In some embodiments, X_{M} is Lu-177. In some embodiments, X_{M} is Ga-68.

In some embodiments, provided herein are conjugates having the same peptide sequence and linker as the conjugates described in Table 2, Table 3A or Table 3B, except that the ring closing linkage between the amino acid residue of position 1 and the cysteine (e.g., at position 12) are covalently bound by a different group. For example, the amino acid residue of position 1 can comprise a group selected from maleimides, halides, disulfides, electron-deficient alkynes, thioesters, and alkenes, which forms a covalent bond with the cysteine.

In some embodiments, the peptides of conjugates of Table 1 are monocyclic peptides, wherein the —C(═O)—Cl at residue position 1 forms a bond with the cysteine at residue position 12. In some embodiments, the peptides of conjugates of Table 1 are monocyclic peptides, wherein the —C(═O)—CH₂—Cl at residue position 1 forms a bond with the cysteine at residue position 12. In some embodiments, the peptides in the conjugates of Table 1 are monocyclic peptides with 12 amino acid residues forming the ring.

In one aspect, described herein is a peptide that has avidity for GPC3, wherein the peptide competes for binding to human GPC3 with a peptide that has an amino acid sequence including deletion, substitution, and/or addition of one or several amino acids in the amino acid of SEQ ID NO: 1:

(SEQ ID NO: 1)
MeK-MeI-D-MeQ-F4COO-I-I-Y-MeNal27N-G-3Py6Ph-MeC

or a pharmaceutically acceptable salt thereof.

In one aspect, described herein is a peptide that has avidity for GPC3, wherein the peptide competes for binding to human GPC3 with a peptide that has a structure of Formula (I) as described herein (e.g., Formula (I-1), (I-2), (I-3), (I-4), and (I-5)), or a pharmaceutically acceptable salt thereof.

In some embodiments, the radiopharmaceutical conjugate has a binding affinity to a human GPC3 of at most 100 nM as determined by Kd in surface plasmon resonance (SPR) analysis. In some embodiments, the radiopharmaceutical conjugate has a binding affinity to a human GPC3 of at most 1 nM as determined by K_{d} in surface plasmon resonance (SPR) analysis.

The structures of exemplary unnatural amino acids that are present in Table 1 can be found in Table 5G.

As described in Table 1 or other tables, abbreviations have the following meanings:
Lower case d means D-amino acids, e.g., dF refers to d-phenylalanine;
Me refers to a methyl group, e.g., MeG represents I-Methyl-Glycine;
Ala or A refer to alanine;
Arg or R refer to arginine;
Asn or N refer to asparagine;
Asp or D refer to aspartic acid;
Cys or C refer to cysteine;
Gln or Q refer to glutatnine;
Gly or G refer to glycine;
His or H refer to histidine;
Ile or L refer to isoleucine;
Leu or L refer to leucine;
Ly s or K refer to lysine;
Met or M refer to methionine;
Phe or F refer to phenylalanine;
Pro or P refer to proline;
Ser or S refer to serine;
Thr or T refer to threonine;
Trp or W refer to tryptophan;
Ty r or Y refer to tyrosine;
Val or V refer to valine;
Unless otherwise stated in the present specification, the following abbreviations for non-natural amino acids are used according to the following meanings;
F4G 2-amino-3-(4-guanidinophenyl)propanoic acid, such as (S)-2-amino-3-(4-guanidinophenyl)propanoic acid (CAS 59574-11-7);
4Py 2-amino-3-(pyridin-4-yl)propanoic acid, such as (S)-2-amino-3-(pyridin-4-yl)propanoic acid (CAS 37535-49-2);
3Py 2-aminxo-3-(pyridin-3-yl)propanoic acid, such as (S)-2-amino-3-(pyridin-3-yl)propanoic acid (CAS 64090-98-8);
Cit 2-amino-S-ureidopentanoic acid, such as (S)-2-amino-S-ureidopentanoic acid (CAS 372-75-8);
KCOpipzaa 2-amino-6-{[4-(carboxymethyl)piperazine-1-carbonyl]amino}hexanoic acid, such as (2S)-2-amino-6-{[4-(carboxymethyl)piperazine-1-carbonylamino}hexanoic acid

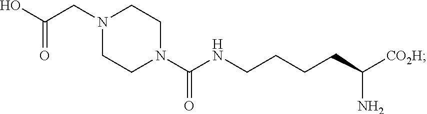 [SMILES: N[C@@H](CCCCNC(=O)N1CCN(CC(=O)O)CC1)C(=O)O]

Eva 2-amino-3-ethylpentanoic acid, such as (S)-2-amino-3-ethyl pentanoic acid (CAS 14328-49-5);
Ahp 2-aminoheptanoic acid, such as (S)-2-aminoheptanoic acid (CAS No. 44902-02-5);
F4COO 4-(2-amino-2-carboxyethyl)benzoic acid, such as (S)-4-(2-amino-2-carboxyethyl)benzoic acid (CAS 126109-42-0);
KCOpip4COO 1-((5-amino-S-carboxypentyl)carbamoyl)piperidine-4-carboxylic acid, such as (S)-1-((5-amino-S-carboxypentyl)carbamoyl)piperidine-4-carboxylic acid

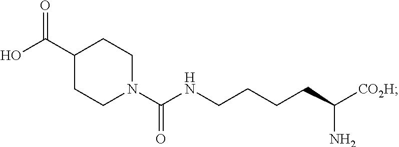 [SMILES: N[C@@H](CCCCNC(=O)N1CCC(C(=O)O)CC1)C(=O)O]

Aib 2-amino-2-methylpropanoic acid (CAS 62-57-7);
Hpr piperidine-2-carboxylic acid, such as (S)-piperidine-2-carboxylic acid (CAS 3105-95-1);
Sbu 2-amino-3-butoxypropanoic acid, such as (2S)-2-amino-3-butoxypropanoic acid (CAS 138320-46-4);
MeDapCOpipzaa 3-(4-(carboxymethyl)piperazine-1-carboxamido)-2-(methylamino)propanoic acid, such as (S)-3-(4-(carboxymethyl)piperazine-1-carboxamido)-2-(methylamino)propanoic acid;

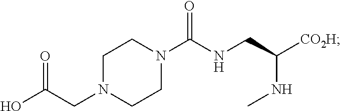 [SMILES: CN[C@@H](CNC(=O)N1CCN(CC(=O)O)CC1)C(=O)O]

Scbm 2-amino-3-(cyclobutylmethoxy)propanoic acid, such as (2S)-2-amino-3-(cyclobutylethyl)propanoic acid

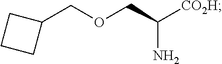 [SMILES: N[C@@H](COCC1CCC1)C(=O)O]

Sbu 2-amino-3-butoxypropanoic acid, such as (2S)-2-amino-3-butoxypropanoic acid (CAS 138320-46-4);
Scpe 2-amino-3-(cyclopentyloxy)propanoic acid, such as (2S)-2-amino-3-(cyclopentyloxy)propanoic acid (CAS 1509921-28-1);
HseBu 2-amino-4-butoxy butanoic acid, such as (2S)-2-amino-4-butoxy butanoic acid (CAS 17673-71-1);
A4paa 2-amino-3-(1-(carboxymethyl)piperidin-4-yl)propanoic acid, such as (S)-2-amino-3-(1-(carboxymethyl)piperidin-4-yl)propanoic acid

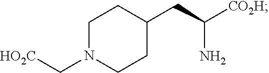 [SMILES: N[C@@H](CC1CCN(CC(=O)O)CC1)C(=O)O]

Spent 2-amino-3-(pentyloxy)propanoic acid, such as (2S)-2-amino-3-(pentyloxy)propanoic acid (CAS 1502644-74-7);
Hg 2-aminohexanedioic acid, such as (S)-2-aminohexanedioic acid (CAS 1118-90-7)
Hsccpe 2-amino-4-(cyclopentyloxy)butanoic acid, such as (2S)-2-amino-4-(cyclopentyloxy)butanoic acid (CAS 1501949-22-9);
Hgn 2,6-diamino-6-oxohexanoic acid, such as (S)-2,6-diamino-6-oxohexanoic acid (CAS 7433-32-1);
DapCOpipzaa 2-amino-3-(4-(carboxymethyl)piperazine-1-carboxamido)propanoic acid, such as (S)-2-amino-3-(4-(carboxymethyl)piperazine-1-carboxamido)propanoic acid

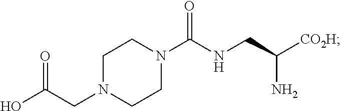 [SMILES: N[C@@H](CNC(=O)N1CCN(CC(=O)O)CC1)C(=O)O]

MeD 2-(methylamine)butanedioic acid, such as (2S)-2-(methylamine)butanedioic acid (CAS 4226-18-0);
CmG 2-[(2-carbamoyl ethyl)amino]acetic acid (CAS 34299-32-6);
Medd 2-(methylamino)butancdioic acid, such as (2R)-2-(methylamine)butanedioic acid (CAS 6384-92-5);
HseEt 2-amino-4-ethoxy butanoic acid, such as (2S)-2-amino-4-methoxybutanoic acid (CAS 17268-93-8);
HseiPr 2-amino-4-(propan-2-yloxy)butanoic acid, such as (2S)-2-amino-4-(propan-2-yloxy)butanoic acid (CAS 685842-10-8);
CrnG 2,2′-azane diyldiacetic acid (CAS 142-73-4);
CeG 3-((carboxymethyl)amino)propanoic acid (CAS 505-72-6);
CrpG 4-((carboxymethyl)amino)butanoic acid (CAS 4386-04-3);
MeHgl 2-(methylamino)hexanedioic acid, such as (S)-2-(methylamino)hexanedioic acid (CAS 261943-13-9);
MeCit 2-(Methylamino)-S-ureidopentanoic acid, such as (S)-2-(methylamine)-S-ureidopentanoic acid (CAS 2226129-88-8);
F3G 2-amino-3-(3-guanidinophenyl)propanoic acid, such as (S)-2-amino-3-(3-guanidinophenyl)propanoic acid (CAS 1019057-42-1);
3Py6COO 5-(2-amino-2-carboxyethyl)picolinic acid, such as (S)-S-(2-amino-2-carboxyethyl)picolinic acid (CAS 1269945-31-4);
4Pv2NH2 2-amino-3-(2-aminopyridin-4-yl)propanoic acid, such as (S)-2-amino-3-(2-aminopyridin-4-yl)propanoic acid (CAS 1269969-46-1);
3Py5COO 5-(2-amino-2-carboxyethyl)nicotinic acid, such as (S)-S-(2-amino-2-carboxyethyl)nicotinic acid (CAS 1270138-35-6);
F3COO 3-(2-amino-2-carboxyethyl)benzoic acid, such as (S)-3-(2-amino-2-carboxyethyl)benzoic acid (CAS 13861-02-4)
3Py6NHAc 3-(6-acetamidopyridin-3-yl)-2-aminopropanoic acid, such as (S)-3-(6-acetamidopyridin-3-yl)-2-aminopropanoic acid (CAS 1270189-35-9);
F4C 2-amino-3-(4-chlorophenyl)propanoic acid, such as (S)-2-amino-3-(4-chlorophenyl)propanoic acid (CAS 14173-39-8)
F40Me 2-amino-3-(4-methoxyphenyl)propanoic acid, such as (S)-2-amino-3-(4-methoxyphenyl)propanoic acid (CAS 6230-11-1);
Nal2 2-amino-3-(naphthalen-2-yl)propanoic acid, such as (S)-2-amino-3-(naphthalen-2-yl)propanoic acid (CAS 58438-03-2);
F3aao 2-amino-3-(3-(carboxymethoxy)phenyl)propanoic acid, such as (S)-2-amino-3-(3-(carboxymethoxy)phenyl)propanoic acid

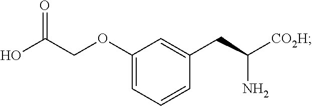 [SMILES: N[C@@H](Cc1cccc(OCC(=O)O)c1)C(=O)O]

F4aa 2-amino-3-(4-(carboxymethyl)phenyl)propanoic acid, such as (S)-2-amino-3-(4-(carboxymethyl)phenyl)propanoic acid (CAS 140233-60-9);
F4aao 2-amino-3-(4-(carboxymethoxy)phenyl)propanoic acid, such as (S)-2-amino-3-(4-(carboxymethoxy)phenyl)propanoic acid (CAS 24558-63-2);
3Py6NHaa 2-amino-3-(6-((carboxymethyl)amino)pyridin-3-yl)propanoic acid, such as (S)-2-amino-3-(6-((carboxymethyl)amino)pyridin-3-yl)propanoic acid

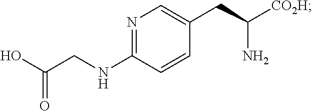 [SMILES: N[C@@H](Cc1ccc(NCC(=O)O)nc1)C(=O)O]

5Pdo 2-amino-3-(6-oxo-1,6-dihydropyridin-3-yl)propanoic acid, such as (S)-2-amino-3-(6-oxo-1,6-dihydropyridin-3-yl)propanoic acid (CAS 140681-92-1);
F3CON 2-amino-3-(3-carbamoylphenyl)propanoic acid, such as (S)-2-amino-3-(3-carbamoylphenyl)propanoic acid (CAS 12¹7651-22-3);
F4F 2-amino-3-(4-fluorophenyl)propanoic acid, such as (S)-2-amino-3-(4-fluorophenyl)propanoic acid (CAS 1132-68-9);
F40Et 2-amino-3-(4-ethoxyphenyl)propanoic acid, such as (S)-2-amino-3-(4-ethoxyphenyl)propanoic acid (CAS 32795-52-1);
F4Mc 2-amino-3-(p-tolyl)propanoic acid, such as (S)-2-amino-3-(p-tolyl)propanoic acid (CAS 1991-87-3);
F4CON 2-amino-3-(4-carbamoylphenyl)propanoic acid, such as (S)-2-amino-3-(4-carbamoylphenyl)propanoic acid (CAS 223593-04-2);
F4CONPEG4Me 3-(4-((2,5,8,11-tetraoxatridecan-13-yl)carbamoyl)phenyl)-2-aminopropanoic acid, such as (S)-3-(4-((2,5,8,11-tetraoxatridecan-13-yl)carbamoyl)phenyl)-2-aminopropanoic acid

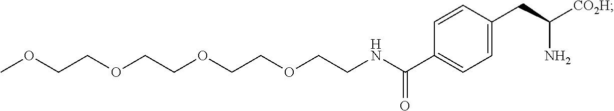 [SMILES: COCCOCCOCCOCCNC(=O)c1ccc(C[C@H](N)C(=O)O)cc1]

F30Me 2-amino-3-(3-methoxyphenyl)propanoic acid, such as (S)-2-amino-3-(3-methoxyphenyl)propanoic acid (CAS 33879-32-2);
YaeCOpipzaa 2-amino-3-(4-(2-(4-(carboxymethyl)piperazine-1-carboxamido)ethoxy)phenyl)propanoic acid, such as (S)-2-amino-3-(4-(2-(4-(carboxymethyl)piperazine-1-carboxamido)ethoxy)phenyl)propanoic acid

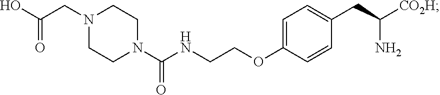 [SMILES: N[C@@H](Cc1ccc(OCCNC(=O)N2CCN(CC(=O)O)CC2)cc1)C(=O)O]

F4aaopipzaa 2-amino-3-(4-(2-(4-(carboxymethyl)piperazin-1-yl)-2-oxoethoxy)phenyl)propanoic acid, such as (S)-2-amino-3-(4-(2-(4-(carboxymethyl)piperazin-1-yl)-2-oxoethoxy)phenyl)propanoic acid

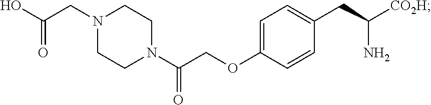 [SMILES: N[C@@H](Cc1ccc(OCC(=O)N2CCN(CC(=O)O)CC2)cc1)C(=O)O]

4Pdo 2-amino-3-(2-oxo-1,2-dihydropyridin-4-yl)propanoic acid, such as (S)-2-amino-3-(2-oxo-1,2-dihydropyridin-4-yl)propanoic acid (CAS 1270061-10-3);
3Py6CON 2-amino-3-(6-carbamoylpyridin-3-yl)propanoic acid, such as (S)-2-amino-3-(6-carbamoylpyridin-3-yl)propanoic acid

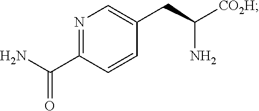 [SMILES: NC(=O)c1ccc(C[C@H](N)C(=O)O)cn1]

Atp 2-amino-3-(tetrahydro-2H-pyran-4-yl)propanoic acid, such as (S)-2-amino-3-(tetrahydro-2H-pyran-4-yl)propanoic acid (CAS 1344910-91-3);
Cha4cH 2-amino-3-((1s,4R)-4-hydroxycyclohexyl)propanoic acid, such as (S)-2-amino-3-((1s,4R)-4-hydroxycyclohexyl)propanoic acid

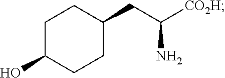 [SMILES: N[C@@H](C[C@H]1CC[C@@H](O)CC1)C(=O)O]

Cha4tH 2-amino-3-((1r,4S)-4-hydroxycyclohexyl)propanoic acid, such as (S)-2-amino-3-((1r,4S)-4-hydroxy cyclohexyl)propanoic acid (CAS 221243-22-7);
Cha4cOMc 2-amino-3-((1s,4R)-4-methoxycyclohexyl)propanoic acid, such as (S)-2-amino-3-((1s,4R)-4-methoxycyclohexyl)propanoic acid

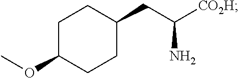

A1mor 2-amino-3-morpholinepropanoic acid, such as (S)-2-amino-3-morpholinepropanoic acid (CAS 1931924-34-3);
F4amCOpipzaa 2-amino-3-(4-((4-(carboxymethyl)piperazine-1-carboxamido)methyl)phenyl)propanoic acid, such as (S)-2-amino-3-(4-((4-(carboxymethyl)piperazine-1-carboxamido)methyl)phenyl)propanoic acid

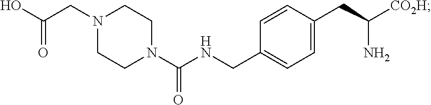 [SMILES: N[C@@H](Cc1ccc(CNC(=O)N2CCN(CC(=O)O)CC2)cc1)C(=O)O]

Chg 2-amino-2-cyclohexylacetic acid, such as (S)-2-amino-2-cyclohexylacetic acid (CAS 14328-51-9);
Cle 1-aminocyclopentane-1-carboxylic acid (CAS 52-52-8).
Tbg 2-amino-3,3-dimethylbutanoic acid, such as (S)-2-amino-3,3-dimethylbutanoic acid (CAS 20859-02-3);
Gcpr 2-amino-2-cyclopropylacetic acid, such as (S)-2-amino-2-cyclopropylacetic acid (CAS 49606-99-7);
Gcpe 2-amino-2-cyclopentylene acid, such as (S)-2-amino-2-cyclopentylacetic acid (CAS 2521-84-8);
Acpr 2-amino-3-cyclopropylpropanoic acid, such as (S)-2-amino-3-cyclopropylpropanoic acid (CAS 102735-53-5);
Cba 2-amino-3-cyclobutylpropanoic acid, such as (S)-2-amino-3-cyclobutylpropanoic acid (CAS 1201593-65-8);
Gthp 2-amino-2-(tetrahydro-2H-pyran-4-yl)acetic acid, such as (S)-2-amino-2-(tetrahydro-2H-pyran-4-yl)acetic acid (CAS 811842-25-8);
NleCOO 2-aminoheptanedioic acid, such as (S)-2-aminoheptanedioic acid (CAS 26630-55-7);
NleOH 2-amino-6-hydroxyhexanoic acid, such as (S)-2-amino-6-hydroxyhexanoic acid (CAS 6033-32-5);
Atb 2-amino-4,4-dlimethylpentanoic acid, such as (S)-2-amino-4,4-dimethylpentanoic acid (CAS 57224-50-7);
Nva 2-aminopentanoic acid, such as (S)-2-aminopentanoic acid (CAS 6600-40-4);
Nle 2-aminohexanoic acid, such as (S)-2-aminohexanoic acid (CAS 327-57-1);
DapAc 3-acetamido-2-aminopropanoic acid, such as (S)-3-acetamido-2-aminopropanoic acid (CAS 20584-70-7);
Abu 2-aminobutanoic acid, such as (S)-2-aminobutanoic acid (CAS 1492-24-6);
Ncit 2-amino-4-ureidobutanoic acid, such as (S)-2-amino-4-ureidobutanoic acid (CAS 1190-47-2);
dMcS 2-amino-3-hydroxy-3-methylbutanoic acid, such as (S)-2-amino-3-hydroxy-3-methylbutanoic acid (CAS 2280-27-5);
TdMe 2-amino-3-methoxy-3-methylbutanoic acid, such as (S)-2-amino-3-methoxy-3-methylbutanoic acid (CAS 2280-29-7);
Cbg 2-amino-2-cyclobutylacetic acid, such as (S)-2-amino-2-cyclobutylacetic acid (CAS 49607-08-1);
NvaOMe2-amino-S-methoxypentanoic acid, such as (S)-2-amino-S-methoxypentanoic acid (CAS 1315051-48-9);
NIeOMe 2-amino-6-methoxyhexanoic acid, such as (S)-2-amino-6-methoxyhexanoic acid (CAS 1690134-89-4);
AhpOMe2-amino-7-mnethoxyheptanoic acid, such as (S)-2-amino-7-methoxyheptanoic acid;
F4u 2-amino-3-(4-ureidophenyl)propanoic acid, such as (S)-2-amino-3-(4-ureidophenyl)propanoic acid (CAS 32401-733-4);
F4CONdMe 2-amino-3-(4-(dimethylcarbamoyl)phenyl)propanoic acid, such as (S)-2-amino-3-(4-(dimethylcarbamoyl)phenyl)propanoic acid (CAS 2349530-31-8);
F4 ms 2-amino-3-(4-(methylsulfonyl)phenyl)propanoic acid, such as (S)-2-amino-3-(4-(methylsulfonyl)phenyl)propanoic acid (CAS 1195655-47-0);
F34dOMe 2-amino-3-(3,4-dimnethoxyphenyl)propanoic acid, such as (S)-2-amino-3-(3,4-dimethoxyphenyl)propanoic acid (CAS 32161-30-1);
F3C 2-amino-3-(3-chlorophenyl)propanoic acid, such as (S)-2-amino-3-(3-chlorophenyl)propanoic acid (CAS 80126-51-8);
F3CONdMe 2-amino-3-(3-(dimethylcarbamoyl)phenyl)propanoic acid, such as (S)-2-amino-3-(3-(dimethylcarbanoyl)phenyl)propanoic acid

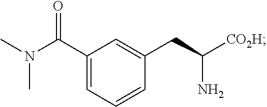 [SMILES: CN(C)C(=O)c1cccc(C[C@H](N)C(=O)O)c1]

51nda 2-amino-3-(1H-indazol-S-yl)propanoic acid, such as (S)-2-amino-3-(1H-indazol-S-yl)propanoic acid (CAS 1335577-14-4);
F3aa 2-amino-3-(3-(carboxymethyl)phenyl)propanoic acid, such as (S)-2-amino-3-(3-(carboxymethyl)phenyl)propanoic acid (CAS 1270107-31-7);
3Py6Nhae 2-amino-3-(6-((2-aminoethyl)amino)pyridin-3-yl)propanoic acid, such as (S)-2-amino-3-(6-((2-aminoethyl)amino)pyridin-3-yl)propanoic acid

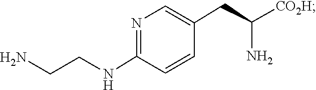 [SMILES: NCCNc1ccc(C[C@H](N)C(=O)O)cn1]

3Py6OMe 2-amino-3-(6-methoxypyridin-3-yl)propanoic acid, such as (S)-2-amino-3-(6-methoxypyridin-3-yl)propanoic acid (CAS 1270178-24-9);
MeNal2 2-(methylamino)-3-(naphthalen-2-yl)propanoic acid, such as (S)-2-(methylamino)-3-(naphthalen-2-v)propanoic acid

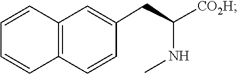 [SMILES: CN[C@@H](Cc1ccc2ccccc2c1)C(=O)O]

MeNa127N 3-(isoquinolin-7-yl)-2-(methylanilino)propanoic acid, such as (S)-3-(isoquinolin-7-yl)-2-(methylamine)propanoic acid

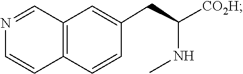 [SMILES: CN[C@@H](Cc1ccc2ccncc2c1)C(=O)O]

MeF34diox 3-(2,3-dihydrobenzo[b][1,4]dioxin-6-yl)-2-(methylamino)propanoic acid, such as (S)-3-(2,3-dihydrobenzo[b][1,4]dioxin-6-yl)-2-(methylamino)propanoic acid

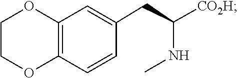 [SMILES: CN[C@@H](Cc1ccc2c(c1)OCCO2)C(=O)O]

MeF34dOMe 3-(3,4-dimethoxyphenyl)-2-(methylamine)propanoic acid, such as (S)-3-(3,4-dimethoxyphenyl)-2-(methylamino)propanoic acid (CAS 52939-34-1);
MeF4T 2-(methylamino)-3-(4-(trifluoromethyl)phenyl)propanoic acid, such as (S)-2-(methylamino)-3-(4-(trifluoromethyl)phenyl)propanoic acid

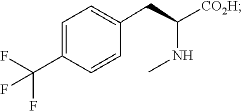 [SMILES: CN[C@@H](Cc1ccc(C(F)(F)F)cc1)C(=O)O]

MeW7N 2-(methylamino)-3-(1H-pyrrolo[2,3-b]pyridin-3-yl)propanoic acid, such as (S)-2-(methylamino)-3-(1H-pyrrolo[2,3-b]pyridin-3-yl)propanoic acid (CAS 356067-71-5).
MeF3C₄Mc 3-(3-chloro-4-methylphenyl)-2-(methylamino)propanoic acid, such as (S)-3-(3-chloro-4-methylphenyl)-2-(methylamino)propanoic acid

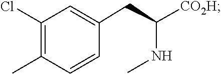

MeF3Me4C 3-(4-chloro-3-methylphenyl)-2-(methylamino)propanoic acid, such as (S)-3-(4-chloro-3-methylphenyl)-2-(methylamino)propanoic acid

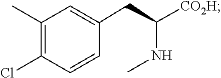

Bph 3-([11′-biphenyl]1-4-yl)-2-aminopropanoic acid, such as (S)-3-([1,1′-biphenyl]-4-yl)-2-aminopropanoic acid (CAS 155760-02-4);
3Py6Ph 2-amino-3-(6-phenylpyridin-3-yl)propanoic acid, such as (S)-2-amino-3-(6-phenylpyridin-3-yl)propanoic acid

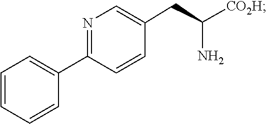 [SMILES: N[C@@H](Cc1ccc(-c2ccccc2)nc1)C(=O)O]

F41Me4Pyz 2-amino-3-(4-(1-methyl-1H-pyrazol-4-yl)phenyl)propanoic acid, such as (S)-2-amino-3-(4-(1-methyl-1H-pyrazol-4-yl)phenyl)propanoic acid

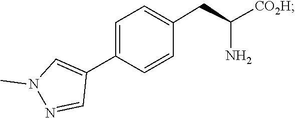 [SMILES: Cn1cc(-c2ccc(C[C@H](N)C(=O)O)cc2)cn1]

F43Pyz 3-(4-(1H-pyrazol-3-yl)phenyl)-2-aminopropanoic acid, such as (S)-3-(4-(1H-pyrazol-3-yl)phenyl)-2-aminopropanoic acid

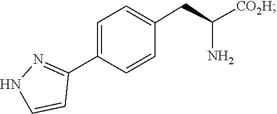 [SMILES: N[C@@H](Cc1ccc(-c2cc[nH]n2)cc1)C(=O)O]

F44Pyz 3-(4-(1H-pyrazol-4-yl)phenyl)-2-aminopropanoic acid, such as (S)-3-(4-(1H-pyrazol-4-yl)phenyl)-2-aminopropanoic acid

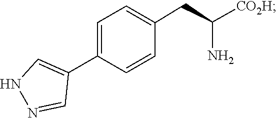 [SMILES: N[C@@H](Cc1ccc(-c2cn[nH]c2)cc1)C(=O)O]

F41Pyz 3-(4-(1H-pyrazol-1-yl)phenyl)-2-aminopropanoic acid, such as (S)-3-(4-(1H-pyrazol-1-yl)phenyl)-2-aminopropanoic acid (CAS 1269982-08-2);
F41Me3Pyz 2-amino-3-(4-(1-methyl-1H-pyrazol-3-yl)phenyl)propanoic acid, such as (S)-2-amino-3-(4-(1-methyl-1H-pyrazol-3-yl)phenyl)propanoic acid

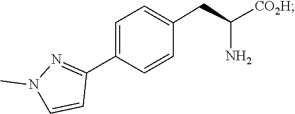 [SMILES: Cn1ccc(-c2ccc(C[C@H](N)C(=O)O)cc2)n1]

F41Et4Pyz 2-amino-3-(4-(1-ethyl-1H-pyrazol-4-yl)phenyl)propanoic acid, such as (S)-2-amino-3-(4-(1-ethyl-1H-pyrazol-4 yl)phenyl)propanoic acid

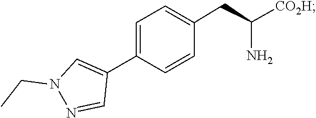 [SMILES: CCn1cc(-c2ccc(C[C@H](N)C(=O)O)cc2)cn1]

F41MeOe4Pyz 2-amino-3-(4-(1-(2-methoxyethyl)-1H-pyrazol-4-yl)phenyl)propanoic acid, such as (S)-2-amino-3-(4-(1-(2-methoxyethyl)-1H-pyrazol-4-yl)phenyl)propanoic acid

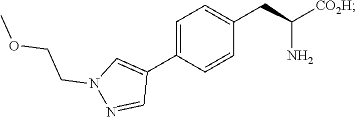 [SMILES: COCCn1cc(-c2ccc(C[C@H](N)C(=O)O)cc2)cn1]

F41McOp4Pyz 2-amino-3-(4-(1-(3-methoxypropyl)-1H-pyrazol-4-yl)phenyl)propanoic acid, such as (S)-2-amino-3-(4-(1-(3-methoxypropyl)-1H-pyrazol-4-yl)phenyl)propanoic acid

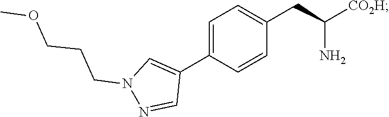 [SMILES: COCCCn1cc(-c2ccc(C[C@H](N)C(=O)O)cc2)cn1]

F44thp 2-amino-3-(4-(tetrahydro-2H-pyran-4-yl)phenyl)propanoic acid, such as (S)-2-amino-3-(4-(tetrahydro-2H-pyran-4-yl)phenyl)propanoic acid

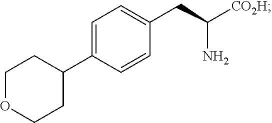 [SMILES: N[C@@H](Cc1ccc(C2CCOCC2)cc1)C(=O)O]

F4Ac4Pip 3-(4-(1-acetylpiperidin-4-yl)phenyl)-2-aminopropanoic acid, such as (S)-3-(4-(1-acetylpiperidin-4-yl)phenyl)-2-aminopropanoic acid

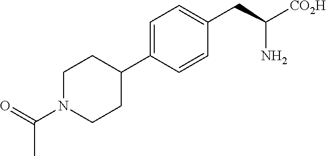 [SMILES: CC(=O)N1CCC(c2ccc(C[C@H](N)C(=O)O)cc2)CC1]

PhNya 2-amino-S-phenylpentanoic acid, such as (S)-2-amino-S-phenylpentanoic acid (CAS 62777-25-7);
PhNle 2-amino-6-phenylhexanoic acid, such as (S)-2-amino-6-phenylhexanoic acid (CAS 80887-26-9);
Yph 2-amino-3-(4-phenoxyphenyl)propanoic acid, such as (S)-2-amino-3-(4-phenoxyphenyl)propanoic acid (CAS 150351-64-7);
Ybn 2-amino-3-(4-(benzyloxy)phenyl)propanoic acid, such as (S)-2-amino-3-(4-(benzyloxy)phenyl)propanoic acid (CAS 16652-64-5);
F4tb 2-amino-3-(4-(tert-butyl)phenyl)propanoic acid, such as (S)-2-amino-3-(4-(tert-butyl)phenyl)propanoic acid

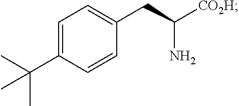 [SMILES: CC(C)(C)c1ccc(C[C@H](N)C(=O)O)cc1]

F4Opr 2-amino-3-(4-propoxyphenyl)propanoic acid, such as (S)-2-amino-3-(4-propoxyphenyl)propanoic acid (CAS 32795-53-2);
S3Ret (3R)-2-amino-3-hydroxypentanoic acid, such as (2S,3R)-2-amino-3-hydroxypentanoic acid (CAS 10148-67-1);
Yae 2-amino-3-(4-(2-aminoethoxy)phenyl)propanoic acid, such as (S)-2-amino-3-(4-(2-aminoethoxy)phenyl)propanoic acid (CAS 1909283-20-0);
datb 2-amino-4,4-dimethylpentanoic acid, such as (R)-2-amino-4,4-dimethylpentanoic acid (CAS 88319-43-);
Me3Py 2-(methylamino)-3-(pyridin-3-yl)propanoic acid, such as (S)-2-(methylamino)-3-(pyridin-3-yl)propanoic acid (CAS 2651172-69-7);
W5N 2-amino-3-(1H-pyrrolo[3,2-c]pyridin-3-yl)propanoic acid, such as (S)-2-amino-3-(III-pyrrolo[3,2-c]pyridin-3-yl)propanoic acid (CAS 149704-62-1)
McA4paa 3-(1-(carboxymethyl)piperidin-4-yl)-2-(methylamino)propanoic acid, such as (S)-3-(1-(carboxymethyl)piperidin-4-yl)-2-(methylamino)propanoic acid

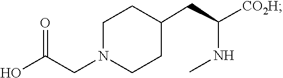 [SMILES: CN[C@@H](CC1CCN(CC(=O)O)CC1)C(=O)O]

MeI (3S)-3-methyl-2-(methylamino)pentanoic acid, such as (2S,3S)-3-methyl-2-(methylamino)pentanoic acid (CAS 4125-98-8);
MeA 2-(methylamino)propanoic acid, such as (2S)-2-(methylamino)propanoic acid (CAS 3913-67-5);
MeG 2-(methylamino)acetic acid, such as 2-(methylamino)acetic acid (CAS 107-97-1);
MeV 3-menthol-2-(dimethylamino)butanoic acid, such as (2S)-3-methyl-2-(methylamino)butanoic acid (CAS 2480-23-1)
MeT (3R)-3-hydroxy-2-(methylamino)butanoic acid, such as (2S,3R)-3-hydroxy-2-(methylamino)butanoic acid (CAS 2812-28-4)
MeC N-methylcysteine, such as (R)-N-methylcysteine
alI (3R)-2-amino-3-methylpentanoic acid, such as (2S,3R)-2-amino-3-methylpentanoic acid (CAS 1509-34-8)
TMe (3R)-2-amino-3-methoxybutanoic acid, such as (2S,3R)-2-amino-3-methoxybutanoic acid (CAS 4144-02-9)
MeQ 4-carbamoyl-2-(methylamino)butanoic acid, such as (2S)-4-carbamoyl-2-(methylamino)butanoic acid (CAS 300560-56-9)
MeTMe (3R)-3-methoxy-2-(methylamino)butanoic acid, such as (2S,3R)-3-methoxy-2-(methylamino)butanoic acid (CAS 594865-59-5)
MeK 6-amino-2-(dimethylamino)hexanoic acid, such as (2S)-6-amino-2-(methylamino)hexanoic acid (CAS 7431-89-2)
MeKAc 6-acetamido-2-(methylamino)hexanoic acid, such as (2S)-6-acetamido-2-(methylamino)hexanoic acid

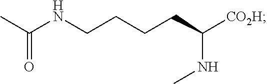

MeK(datb) N6-(2-amino-4,4-dimethylpentanol)-N2-methyl-lysine, such as N6-((R)-2-amino-4,4-dimethylpentanoyl)-N2-methyl-L-lysine

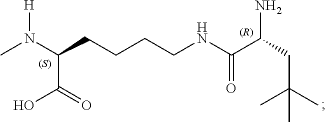

MeK(de)N6-(glutamyl)-N2-methyl-lysine, such as N6-(D-glutamyl)-N2-methyl-L-lysine

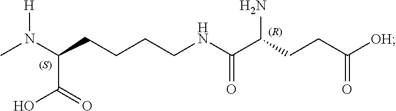

MeK(df) N6-(phenylalanyl)-N2-methyl-lysine, such as N6-(D-phenylalanyl)-N2-methyl-L-lysine

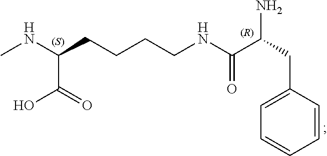

MeK(H) N6-(histidyl)-N2-diethyl-lysine, such as N6-(L-histidyl)-N2-methyl-L-lysine

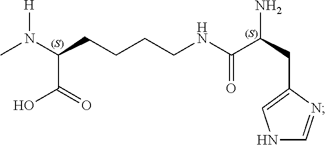

Har 2-amino-6-carbamimidanidohexanoic acid, such as (2S)-2-amino-6-carbamimidainidohexanoic acid (CAS 156-86-5)
KAc 2-amino-6-acetamidohexanoic acid, such as (2S)-2-amino-6-acetamidohexanoic acid (CAS 692-04-6)
dd 2-aminobutanedioic acid, such as (2R)-2-aminobutanedioic acid
EtG 2-(ethylamino)acetic acid (CAS 627-01-0)
K(biotin)6-{5-[(3aS,4S,6aR)-2-oxo-hexahydro-1H-thieno[3,4-d]imidazol-4-yl]pentanamido}-2-aminohexanoic acid, such as (2S)-6-{5-[(3aS,4S,6aR)-2-oxo-hexahydro-1H-thieno[3,4-d]imidazol-4-yl]pentanamido}-2-aminohexanoic acid (CAS 576-19-2);
MeeG 2-[(2-methoxyethyl)amino]acetic acid (CAS 205124-55-6);
CmpG 2-[(3-carbamoylpropyl)amino]acetic acid (CAS 1182825-67-7)
Nmm 2-amino-3-(methylcarbamoyl)propanoic acid, such as (2S)-2-amino-3-(methylcarbamoyl)propanoic acid (CAS 149204-93-3)
Ndm 2-amino-3-(dimethylcarbamoyl)propanoic acid, such as (2S)-2-amino-3-(dimethylcarbamoyl)propanoic acid (CAS 138585-02-1);
SMe 2-amino-3-methoxypropanoic acid, such as (2S)-2-amino-3-methoxypropanoic acid (CAS 32620-11-4);
HseMe 2-amino-4-methoxybutanoic acid, such as (2S)-2-amino-4-methoxybutanoic acid (CAS 3311-01-1);
SiPr 2-amino-3-(propan-2-yloxy)propanoic acid, such as (2S)-2-amino-3-(propan-2-yloxy)propanoic acid (CAS 83824-92-4)
SPr 2-amino-3-propoxy propanoic acid, such as (2S)-2-amino-3-propoxy propanoic acid (CAS 113576-31-1)
MeW1Me 3-(1-methyl-1H-indol-3-yl)-2-(methylamino)propanoic acid, such as (2S)-3-(1-methyl-1H-indol-3-yl)-2-(methylamino)propanoic acid (CAS 1152412-98-0)
MeQdMe 4-(dimethylcarbamoyl)-2-(methylamino)butanoic, (2S)-4-(dimethylcarbamoyl)-2-(methylamino)butanoic (CAS 2255323-71-6)
MeSMe 3-methoxy-2-(methylamino)propanoic acid, such as (2S)-3-methoxy-2-(methylamino)propanoic acid

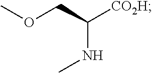

MeHseMe 4-methoxy-2-(methylamino)butanoic acid, such as (2S)-4-methoxy-2-(methylamino)butanoic acid (CAS 1500648-79-2)
MeKCOpipzaa (2S)-6-{[4-(carboxymethyl)piperazine-1-carbonyl]amino}-2-(methylamino)hexanoic acid

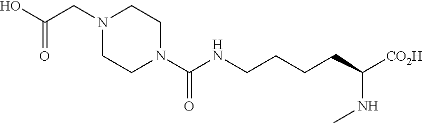 [SMILES: CN[C@@H](CCCCNC(=O)N1CCN(CC(=O)O)CC1)C(=O)O]

KCOpipzetOH 2-amino-6-{[4-(2-hydroxyethyl)piperazine-1-carbonyl]amino}hexanoic acid, such as (2S)-2-amino-6-{[4-(2-hydroxyethyl)piperazine-1-carbonyl]amino}hexanoic acid

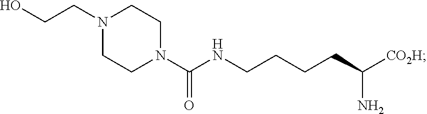 [SMILES: N[C@@H](CCCCNC(=O)N1CCN(CCO)CC1)C(=O)O]

KCOmneglumnine 2-amino-6-({methyl](2S,3R,4R,5R)-2,3,4,5,6-pentahydroxyhexyl]carbamoyl}amino)hexanoic acid, such as (2S)-2-amino-6-({methyl[(2S,3R,4R,5R)-2,3,4,5,6-pentahydroxyhexyl]carbanoyl}amino)hexanoic acid

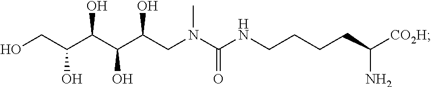 [SMILES: CN(C[C@H](O)[C@@H](O)[C@H](O)[C@H](O)CO)C(=O)NCCCC[C@H](N)C(=O)O]

MeK(SulfoCy5) 2-[(1E,3E)-S-[(2E)-1-(5-{[(5S)-S-carboxy-S-(methylamino)pentyl]carbamoyl}pentyl)-3,3-dimethyl-S-sulfo-2,3-dihydro-1H-indol-2-ylidene]penta-1,3-dien1-1-yl]-1-ethyl-3,3-dimethyl-3H-indol-1-ium-S-sulfonate

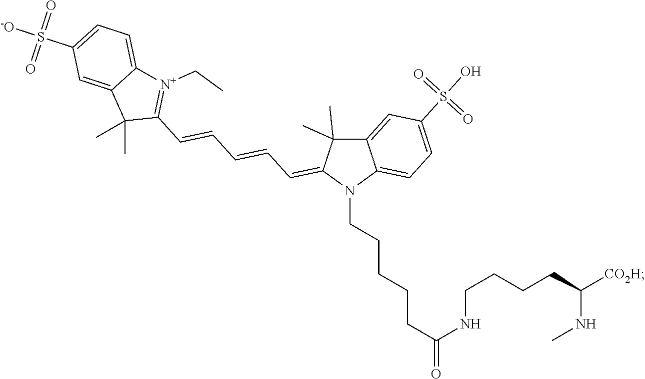

Yae(SulfoCy5) 2-[(1E,3E)-S-[(2E)-1-{5-[(2-{4-[(2S)-2-amino-2-carboxyethyl]phenoxy}ethyl)carbamoyl]pentyl}-3,3-dimethyl-S-sulfo-2,3-dihydro-1H-indol-2-ylidene]penta-1,3-dien-1-yl]-1-ethyl-3,3-dimethyl-3H-indol-1-ium-S-sulfonate

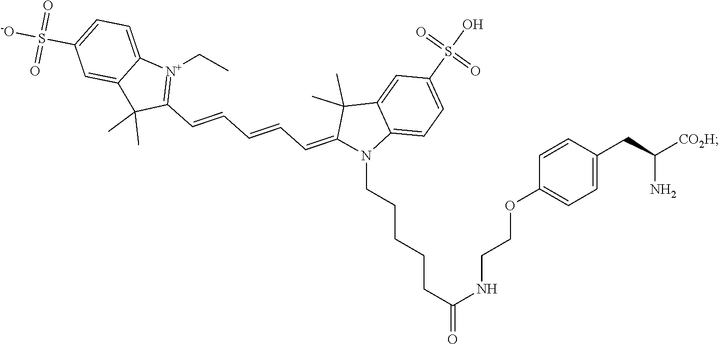

Negative control (FACS assay): 2-[(1E,3E)-S-[(2E)-1-(5-{[(5S)-S-carbamoyl-S—[1-(2-acetamidoacetamido)-3,6,9,12,15,18,21,24,27,30-decaoxatritriacontan-33-amido]pentyl]carbamoyl}pentyl)-3,3-dimethyl-S-sulfo-2,3-dihydro-1H-indol-2-ylidene]penta-1,3-dien-1-yl]-1-ethyl-3,3-dimethyl-3H-indol-1-ium-S-sulfonate

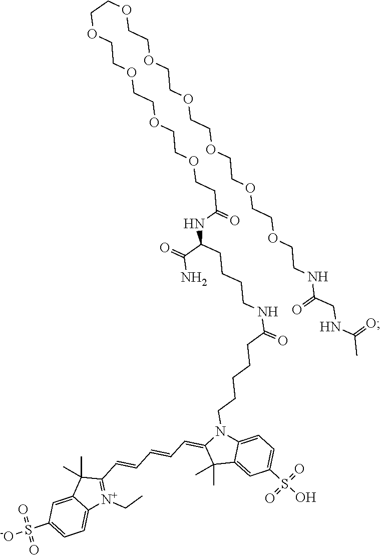

MeK(Biotin-PEG12c) 51-(methylamino)-5,45-dioxo-1-((3aS,4S,6aR)-2-oxohexahydro-1H-thieno[3,4-d]imidazol-4-yl)-9,12,15,18,21,24,27,30,33,36,39,42-dodecaoxa-6,46-diazadopentacontan-52-oic acid, such as (S)-51-(methylamino)-5,45-dioxo-1-((3aS,4S,6aR)-2-oxohexahydro-1H-thieno[3,4-d]imidazol-4-yl)-9,12,15,18,21,24,27,30,33,36,39,42-dodecaoxa-6,46-diazadopentacontan-52-oic acid

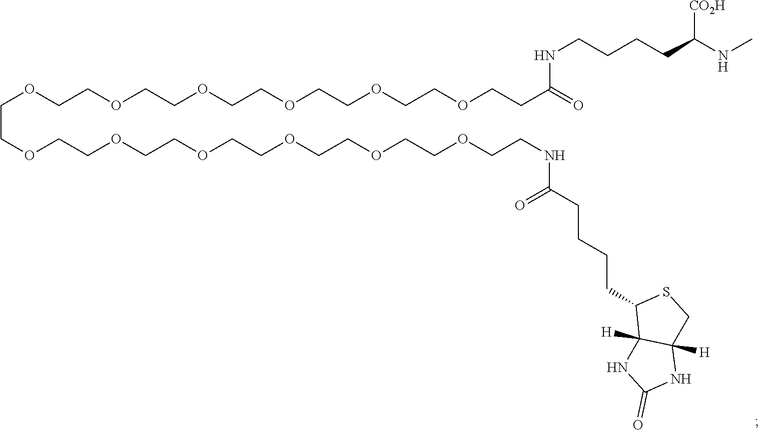 [SMILES: CN[C@@H](CCCCNC(=O)CCOCCOCCOCCOCCOCCOCCOCCOCCOCCOCCOCCOCCNC(=O)CCCC[C@@H]1SC[C@@H]2NC(=O)N[C@@H]21)C(=O)O]

Yae(Biotin-PEG12c) 2-amino-3-(4-((4,44-dioxo-48-((3aS,4S,6aR)-2-oxohexahydro-1H-thieno[3,4-d]imidazol-4-yl)-7,106,5,19,22,25,283,31,337,40-dodecaoxa-3,43-diazaoctatetracontyl)oxy)phenyl)propanoic acid, such as (S)-2-amino-3-(4-((4,44-dioxo-48-((3aS,4 hS,6aR)-2-oxohexahydro-1H-thieno[3,4-d]imidazol-4-yl)-7,10,13,16,19,22,25,28,31,34,37,40-dodecaoxa-3,43-diazaoctatetracontyl)oxy)phenyl)propaoic acid

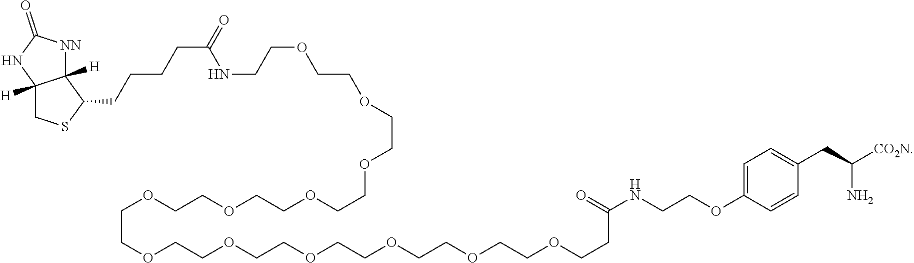

TABLE 1
Exemplary peptide sequences with avidity to GPC3 “Term”
refers to the functional group at the C-terminus.

| | Peptide Sequence | | | | | | |
|---|---|---|---|---|---|---|---|
| SEQ | (from residue position 1 to residue position 15, if present) | | | | | | |
| ID No. | 1 | 2 | 3 | 4 | 5 | 6 | 7 |
| 1 | MeK | MeI | D | MeQ | F4COO | I | I |
| 2 | MeKAc | MeI | D | MeQ | F4COO | I | I |
| 3 | MeKAc | MeI | D | MeQ | F4COO | I | I |
| 4 | MeKAc | MeI | Q | MeQ | F4COO | I | I |
| 5 | MeK | MeI | D | MeQ | F4COO | I | I |
| 6 | MeK | MeI | Q | MeQ | F4COO | I | I |
| 7 | MeKAc | MeI | D | MeQ | F4COO | I | I |
| 8 | MeQ | MeQ | D | MeQ | F4COO | I | I |
| 9 | MeA | MeQ | D | MeQ | F4COO | I | I |
| 10 | MeQ | MeL | D | MeQ | F4COO | I | I |
| 11 | MeQ | MeHseMe | D | MeQ | F4COO | I | I |
| 12 | MeQ | HseMe | D | MeQ | F4COO | I | I |
| 13 | MeQ | MeQ | Hgn | MeQ | F4COO | I | I |
| 14 | MeQ | MeQ | KAc | MeQ | F4COO | I | I |
| 15 | MeQ | MeQ | D | MeA4paa | F4COO | I | I |
| 16 | MeQ | MeQ | D | MeD | F4COO | I | I |
| 17 | MeQ | MeQ | D | MeQ | F4aao | I | I |
| 18 | MeQ | MeQ | D | MeQ | F4COO | TMe | I |
| 19 | MeQ | MeQ | D | MeQ | F4COO | I | I |
| 20 | MeQ | MeQ | D | MeQ | F4COO | I | I |
| 21 | MeK | MeHseMe | D | MeQ | F4COO | I | I |
| 22 | MeK | MeHseMe | D | MeQ | F4COO | I | I |
| 23 | MeK | MeHseMe | D | MeA4paa | F4COO | I | I |
| 24 | MeK | MeQ | D | MeQ | F4COO | TMe | I |
| 25 | MeK | MeQ | D | MeA4paa | F4COO | TMe | I |
| 26 | MeQ | MeQ | D | MeK | F4COO | I | I |
| 27 | MeQ | MeQ | D | MeQ | Yae | I | I |
| 28 | MeQ | MeQ | D | MeQ | F4COO | I | I |
| 29 | MeK | MeQ | Hgn | MeQ | F4COO | TMe | I |
| 30 | MeK | MeI | D | MeQ | F4COO | TMe | I |
| 31 | MeK | MeI | D | MeQ | F4COO | TMe | I |
| 32 | MeK | MeI | D | MeQ | F4COO | TMe | I |
| 33 | MeK(de) | MeI | D | MeQ | F4COO | I | I |
| 34 | MeK(H) | MeI | D | MeQ | F4COO | I | I |
| 35 | MeK | MeQ | D | MeQ | F4COO | TMe | I |
| 36 | MeQ | MeHseMe | D | MeQ | F4COO | TMe | I |
| 37 | MeQ | MeHseMe | D | MeQ | F4COO | TMe | I |
| 38 | MeQ | MeHseMe | D | MeQ | F4COO | TMe | I |
| 39 | MeQ | MeHseMe | D | MeQ | F4COO | TMe | I |
| 40 | MeQ | MeHseMe | D | MeQ | F4COO | TMe | I |
| 41 | MeK(df) | MeI | D | MeQ | F4COO | I | I |
| 42 | MeK(datb) | MeI | D | MeQ | F4COO | I | I |
| 43 | MeA | MeQ | D | MeQ | F4COO | I | I |
| 44 | MeA | MeHseMe | D | MeQ | F4COO | TMe | I |
| 45 | MeK | MeHseMe | D | MeQ | F4COO | TMe | I |
| 46 | MeK | MeKCOpipzaa | D | MeDapCOpipzaa | F4COO | TMe | I |
| 47 | MeK | MeKCOpipzaa | D | MeDapCOpipzaa | 3Py6NHaa | TMe | I |
| 48 | MeK | MeKCOpipzaa | D | MeKCOpipzaa | F4COO | TMe | I |
| 49 | MeK | MeKCOpipzaa | D | MeKCOpipzaa | F4COO | TMe | I |
| 50 | MeDapCOpipzaa | MeHseMe | D | MeDapCOpipzaa | F4COO | TMe | I |
| 51 | MeDapCOpipzaa | MeHseMe | D | MeDapCOpipzaa | 3Py6NHaa | TMe | I |
| 52 | MeK | MeHseMe | D | MeDapCOpipzaa | F4COO | TMe | I |
| 53 | MeK | MeHseMe | D | MeDapCOpipzaa | 3Py6NHaa | TMe | I |
| 54 | MeDapCOpipzaa | MeHseMe | D | MeDapCOpipzaa | F4COO | TMe | I |
| 55 | MeKCOpipzaa | MeHseMe | D | MeDapCOpipzaa | F4COO | TMe | I |
| 56 | MeKCOpipzaa | MeA | D | MeY | F4COO | TMe | I |
| 57 | MeKCOpipzaa | MeA | MeY | D | F4COO | TMe | I |
| 58 | MeKCOpipzaa | MeHseMe | D | MeQ | F4COO | TMe | I |
| 59 | MeKCOpipzaa | MeHseMe | D | MeQ | F4COO | S3REt | I |
| 60 | MeKAc | MeI | D | MeQ | F4COO | I | I |
| 61 | MeK | MeHseMe | D | MeQ | F4COO | I | I |
| 62 | MeK | MeI | D | MeQ | F4COO | TMe | I |
| 63 | MeK | MeHseMe | D | MeQ | F4COO | TMe | I |
| 64 | MeK | MeI | D | MeQ | F4COO | I | I |
| 65 | MeK | MeQ | Hgn | MeQ | F4COO | TMe | I |
| 66 | MeA | MeY | | D | Y | TMe | I |
| 67 | MeA | S | | D | Y | I | I |
| 68 | MeA | S | | W5N | Y | I | I |
| 69 | MeA | Me3Py | | D | Y | all | I |
| 70 | MeA | S | | Q | Y | I | I |
| 71 | MeA | TMe | | D | Y | Cle | I |
| 72 | MeK | MeHseMe | D | MeQ | F4COO | I | I |

| | Peptide Sequence | | | | | | | | |
|---|---|---|---|---|---|---|---|---|---|
| SEQ | (from residue position 1 to residue position 15, if present) | | | | | | | | |
| ID No. | 8 | 9 | 10 | 11 | 12 | 13 | 14 | 15 | Term |
| 1 | Y | MeNal27N | G | 3Py6Ph | MeC | | | | NH₂ |
| 2 | F4COO | MeNal2 | G | 3Py6Ph | MeC | | | | NH₂ |
| 3 | F4COO | MeNal2 | G | F41Me4Pyz | MeC | | | | NH₂ |
| 4 | F4COO | MeNal2 | G | F41Me4Pyz | MeC | | | | NH₂ |
| 5 | F4COO | MeNal2 | G | F41Me4Pyz | MeC | | | | NH₂ |
| 6 | F4COO | MeNal2 | G | F41Me4Pyz | MeC | | | | NH₂ |
| 7 | Y | MeNal27N | G | 3Py6Ph | MeC | | | | NH₂ |
| 8 | F4COO | MeNal2 | G | F41Me4Pyz | MeC | | | | NH₂ |
| 9 | F4COO | MeNal2 | G | F41Me4Pyz | MeC | | | | NH₂ |
| 10 | F4COO | MeNal2 | G | F41Me4Pyz | MeC | | | | NH₂ |
| 11 | F4COO | MeNal2 | G | F41Me4Pyz | MeC | | | | NH₂ |
| 12 | F4COO | MeNal2 | G | F41Me4Pyz | MeC | | | | NH₂ |
| 13 | F4COO | MeNal2 | G | F41Me4Pyz | MeC | | | | NH₂ |
| 14 | F4COO | MeNal2 | G | F41Me4Pyz | MeC | | | | NH₂ |
| 15 | F4COO | MeNal2 | G | F41Me4Pyz | MeC | | | | NH₂ |
| 16 | F4COO | MeNal2 | G | F41Me4Pyz | MeC | | | | NH₂ |
| 17 | F4COO | MeNal2 | G | F41Me4Pyz | MeC | | | | NH₂ |
| 18 | F4COO | MeNal2 | G | F41Me4Pyz | MeC | | | | NH₂ |
| 19 | F4aa | MeNal2 | G | F41Me4Pyz | MeC | | | | NH₂ |
| 20 | F4aao | MeNal2 | G | F41Me4Pyz | MeC | | | | NH₂ |
| 21 | F4aa | MeNal2 | G | 3Py6Ph | MeC | | | | NH₂ |
| 22 | F4aa | MeNal2 | G | F41Me4Pyz | MeC | | | | NH₂ |
| 23 | F4aa | MeNal2 | G | F41Me4Pyz | MeC | | | | NH₂ |
| 24 | F4aa | MeNal2 | G | F41Me4Pyz | MeC | | | | NH₂ |
| 25 | F4aa | MeNal2 | G | F41Me4Pyz | MeC | | | | NH₂ |
| 26 | F4aa | MeNal2 | G | F41Me4Pyz | MeC | | | | NH₂ |
| 27 | F4aa | MeNal2 | G | F41Me4Pyz | MeC | | | | NH₂ |
| 28 | Yae | MeNal2 | G | F41Me4Pyz | MeC | | | | NH₂ |
| 29 | Y | MeNal2 | G | F41Me4Pyz | MeC | | | | NH₂ |
| 30 | F4aao | MeNal27N | G | 3Py6Ph | MeC | | | | NH₂ |
| 31 | F4aao | MeNal27N | G | 3Py6Ph | MeC | de | | | NH₂ |
| 32 | F4aao | MeNal27N | G | 3Py6Ph | MeC | E | | | NH₂ |
| 33 | F4aao | MeNal27N | G | 3Py6Ph | MeC | | | | NH₂ |
| 34 | F4aao | MeNal27N | G | 3Py6Ph | MeC | | | | NH₂ |
| 35 | F4aao | MeNal27N | G | 3Py6Ph | MeC | | | | NH₂ |
| 36 | F4aao | MeNal27N | G | F41Me4Pyz | MeC | | | | NH₂ |
| 37 | YaeCOpipzaa | MeNal2 | G | 3Py6Ph | MeC | | | | NH₂ |
| 38 | F4amCOpipzaa | MeNal2 | G | 3Py6Ph | MeC | | | | NH₂ |
| 39 | F4u | MeNal2 | G | 3Py6Ph | MeC | | | | NH₂ |
| 40 | F4aaopipzaa | MeNal2 | G | 3Py6Ph | MeC | | | | NH₂ |
| 41 | F4aao | MeNal27N | G | 3Py6Ph | MeC | | | | NH₂ |
| 42 | F4aao | MeNal27N | G | 3Py6Ph | MeC | | | | NH₂ |
| 43 | F4COO | MeNal2 | G | 3Py6Ph | MeC | | | | NH₂ |
| 44 | Yae | MeNal2 | G | 3Py6Ph | MeC | | | | NH₂ |
| 45 | Y | MeNal27N | G | 3Py6Ph | MeC | | | | NH₂ |
| 46 | F4aao | MeNal27N | G | 3Py6Ph | MeC | | | | NH₂ |
| 47 | F4aao | MeNal27N | G | 3Py6Ph | MeC | | | | NH₂ |
| 48 | YaeCOpipzaa | MeNal27N | G | 3Py6Ph | MeC | | | | NH₂ |
| 49 | F4aaopipzaa | MeNal27N | G | 3Py6Ph | MeC | | | | NH₂ |
| 50 | Yae | MeNal2 | G | 3Py6Ph | MeC | | | | NH₂ |
| 51 | Yae | MeNal27N | G | 3Py6Ph | MeC | | | | NH₂ |
| 52 | F4aao | MeNal27N | G | 3Py6Ph | MeC | | | | NH₂ |
| 53 | F4aao | MeNal27N | G | 3Py6Ph | MeC | | | | NH₂ |
| 54 | Yae | MeNal27N | G | 3Py6Ph | MeC | | | | NH₂ |
| 55 | Yae | MeNal27N | G | 3Py6Ph | MeC | | | | NH₂ |
| 56 | Yae | MeY | G | 3Py6Ph | MeC | | | | NH₂ |
| 57 | Yae | MeY | G | 3Py6Ph | MeC | | | | NH₂ |
| 58 | Y | MeNal27N | G | 3Py6Ph | MeC | | | | NH₂ |
| 59 | Y | MeNal27N | G | 3Py6Ph | MeC | | | | NH₂ |
| 60 | Y | MeNal27N | G | 3Py6Ph | MeC | G | PEG10c | K | NH₂ |
| 61 | F4aao | MeNal27N | G | 3Py6Ph | MeC | | | | NH₂ |
| 62 | Y | MeNal27N | G | 3Py6Ph | MeC | | | | NH₂ |
| 63 | F4aao | MeNal27N | G | 3Py6Ph | MeC | | | | NH₂ |
| 64 | F4aao | MeNal27N | G | 3Py6Ph | MeC | | | | NH₂ |
| 65 | F4aa | MeNal2 | G | F41Me4Pyz | MeC | | | | NH₂ |
| 66 | Y | MeNal2 | G | 3Py6Ph | MeC | | | | NH₂ |
| 67 | Y | MeNal2 | G | 3Py6Ph | MeC | | | | NH₂ |
| 68 | Y | MeNal2 | G | 3Py6Ph | MeC | | | | NH₂ |
| 69 | Y | MeNal2 | G | 3Py6Ph | MeC | | | | NH₂ |
| 70 | Y | MeNal2 | G | 3Py6Ph | MeC | | | | NH₂ |
| 71 | Y | MeNal2 | G | 3Py6Ph | MeC | | | | NH₂ |
| 72 | Y | MeNal27N | G | 3Py6Ph | MeC | | | | NH₂ |

TABLE 2
Exemplary conjugates of the present disclosure containing a chelated cold Lutetium (Lu-
175) (12mer cyclic peptides). “Term” refers to the functional group at the C-terminus.

| | SEQ | | | | | | |
|---|---|---|---|---|---|---|---|
| Conjugate ID | ID NO. | 1 | 2 | 3 | 4 | 5 | 6 |
| Lu-C-1 | 240 | MeK (DOTA-Lu) | MeI | D | MeQ | F4COO | I |
| Lu-C-21 | 241 | MeK (DOTA-Lu) | MeHseMe | D | MeQ | F4COO | I |
| Lu-C-22 | 242 | MeK (DOTA-Lu) | MeHseMe | D | MeQ | F4COO | I |
| Lu-C-24 | 243 | MeK (DOTA-Lu) | MeQ | D | MeQ | F4COO | TMe |
| Lu-C-28 | 244 | MeQ | MeQ | D | MeQ | F4COO | I |
| Lu-C-26 | 245 | MeQ | MeQ | D | MeK (DOTA-Lu) | F4COO | I |
| Lu-C-30 | 246 | MeK (DOTA-Lu) | MeI | D | MeQ | F4COO | TMe |
| Lu-C-31 | 247 | MeK (DOTA-Lu) | MeI | D | MeQ | F4COO | TMe |
| Lu-C-32 | 248 | MeK (DOTA-Lu) | MeI | D | MeQ | F4COO | TMe |
| Lu-C-33 | 249 | MeK(de) (DOTA-Lu) | MeI | D | MeQ | F4COO | I |
| Lu-C-34 | 250 | MeK(H) (DOTA-Lu) | MeI | D | MeQ | F4COO | I |
| Lu-C-35 | 251 | MeK (DOTA-Lu) | MeQ | D | MeQ | F4COO | TMe |
| Lu-C-44 | 252 | MeA | MeHseMe | D | MeQ | F4COO | TMe |
| Lu-C-45 | 253 | MeK (DOTA-Lu) | MeHseMe | D | MeQ | F4COO | TMe |
| Lu-C-41 | 254 | MeK(df) (DOTA-Lu) | MeI | D | MeQ | F4COO | I |
| Lu-C-42 | 255 | MeK(datb) (DOTA-Lu) | MeI | D | MeQ | F4COO | I |
| Lu-C-64 | 256 | MeK (DOTA-Lu) | MeI | D | MeQ | F4COO | I |
| Lu-C-62 | 257 | MeK (DOTA-Lu) | MeI | D | MeQ | F4COO | TMe |
| Lu-C-72 | 258 | MeK (DOTA-Lu) | MeHseMe | D | MeQ | F4COO | I |
| Lu-C-63 | 259 | MeK (DOTA-Lu) | MeHseMe | D | MeQ | F4COO | TMe |

| Conjugate ID | 7 | 8 | 9 | 10 | 11 | 12 | 13 | Term |
|---|---|---|---|---|---|---|---|---|
| Lu-C-1 | I | Y | MeNal27N | G | 3Py6Ph | MeC | | —NH₂ |
| Lu-C-21 | I | F4aa | MeNal2 | G | 3Py6Ph | MeC | | —NH₂ |
| Lu-C-22 | I | F4aa | MeNal2 | G | F41Me4Pyz | MeC | | —NH₂ |
| Lu-C-24 | I | F4aa | MeNal2 | G | F41Me4Pyz | MeC | | —NH₂ |
| Lu-C-28 | I | Yae (DOTA-Lu) | MeNal2 | G | F41Me4Pyz | MeC | | —NH₂ |
| Lu-C-26 | I | F4aa | MeNal2 | G | F41Me4Pyz | MeC | | —NH₂ |
| Lu-C-30 | I | F4aao | MeNal27N | G | 3Py6Ph | MeC | | —NH₂ |
| Lu-C-31 | I | F4aao | MeNal27N | G | 3Py6Ph | MeC | de | —NH₂ |
| Lu-C-32 | I | F4aao | MeNal27N | G | 3Py6Ph | MeC | F | —NH₂ |
| Lu-C-33 | I | F4aao | MeNal27N | G | 3Py6Ph | MeC | | —NH₂ |
| Lu-C-34 | I | F4aao | MeNal27N | G | 3Py6Ph | MeC | | —NH₂ |
| Lu-C-35 | I | F4aao | MeNal27N | G | 3Py6Ph | MeC | | —NH₂ |
| Lu-C-44 | I | Yae (DOTA-Lu) | MeNal2 | G | 3Py6Ph | MeC | | —NH₂ |
| Lu-C-45 | I | Y | MeNal27N | G | 3Py6Ph | MeO | | —NH₂ |
| Lu-C-41 | I | F4aao | MeNal27N | G | 3Py6Ph | MeC | | —NH₂ |
| Lu-C-42 | I | F4aao | MeNal27N | G | 3Py6Ph | MeC | | —NH₂ |
| Lu-C-64 | I | F4aao | MeNal27N | G | 3Py6Ph | MeC | | —NH₂ |
| Lu-C-62 | I | Y | MeNal27N | G | 3Py6Ph | MeC | | —NH₂ |
| Lu-C-72 | I | Y | MeNal27N | G | 3Py6Ph | MeC | | —NH₂ |
| Lu-C-63 | I | F4aao | MeNal27N | G | 3Py6Ph | MeC | | —NH₂ |

Table 3A illustrates exemplary conjugates of the present disclosure containing a chelated Lutetium-177. As an example, ¹⁷⁷Lu-C-1 of Table 3A has the same structure as Lu-C-1 of Table 2, except that Lu-177 is present in ¹⁷⁷Lu-C-1 while Lu-175 is present in Lu-C-1.

Table 3B illustrates exemplary conjugates of the present disclosure containing a chelated Actinium-225. As an example, ²²⁵Ac-C-1 of Table 3B has the same structure as Lu-C-1 of Table 2, except that Ac-225 is present in ²²⁵Ac-C-1 while Lu-175 is present in Lu-C-1.

TABLE 3A

Conjugate structure (See Table 2;
wherein the Lu-175 from a conjugate
of Table 2 is replaced with Lu-177)

| | Corresponding | Radionuclide bound |
|---|---|---|
| Conjugate | conjugate structure | to the chelator |
| ID | from Table 2 | (replacing Lu-175) |
| ¹⁷⁷Lu—C-1 | Lu—C-1 | ¹⁷⁷Lu |
| ¹⁷⁷Lu—C-21 | Lu—C-21 | ¹⁷⁷Lu |
| ¹⁷⁷Lu—C-22 | Lu—C-22 | ¹⁷⁷Lu |
| ¹⁷⁷Lu—C-24 | Lu—C-24 | ¹⁷⁷Lu |
| ¹⁷⁷Lu—C-28 | Lu—C-28 | ¹⁷⁷Lu |
| ¹⁷⁷Lu—C-26 | Lu—C-26 | ¹⁷⁷Lu |
| ¹⁷⁷Lu—C-30 | Lu—C-30 | ¹⁷⁷Lu |
| ¹⁷⁷Lu—C-31 | Lu—C-31 | ¹⁷⁷Lu |
| ¹⁷⁷Lu—C-32 | Lu—C-32 | ¹⁷⁷Lu |
| ¹⁷⁷Lu—C-33 | Lu—C-33 | ¹⁷⁷Lu |
| ¹⁷⁷Lu—C-34 | Lu—C-34 | ¹⁷⁷Lu |
| ¹⁷⁷Lu—C-35 | Lu—C-35 | ¹⁷⁷Lu |
| ¹⁷⁷Lu—C-44 | Lu—C-44 | ¹⁷⁷Lu |
| ¹⁷⁷Lu—C-45 | Lu—C-45 | ¹⁷⁷Lu |
| ¹⁷⁷Lu—C-41 | Lu—C-41 | ¹⁷⁷Lu |
| ¹⁷⁷Lu—C-42 | Lu—C-42 | ¹⁷⁷Lu |
| ¹⁷⁷Lu—C-64 | Lu—C-64 | ¹⁷⁷Lu |
| ¹⁷⁷Lu—C-62 | Lu—C-62 | ¹⁷⁷Lu |
| ¹⁷⁷Lu—C-72 | Lu—C-72 | ¹⁷⁷Lu |
| ¹⁷⁷Lu—C-63 | Lu—C-63 | ¹⁷⁷Lu |

TABLE 3B

Conjugate structure (See Table 2;
wherein the Lu-175 from a conjugate
of Table 2 is replaced with Ac-225)

| | Corresponding | Radionuclide bound |
|---|---|---|
| Conjugate | conjugate structure | to the chelator |
| ID | from Table 2 | (replacing Lu-175) |
| ²²⁵Ac—C-1 | Lu—C-1 | ²²⁵Ac |
| ²²⁵Ac—C-21 | Lu—C-21 | ²²⁵Ac |
| ²²⁵Ac—C-22 | Lu—C-22 | ²²⁵Ac |
| ²²⁵Ac—C-24 | Lu—C-24 | ²²⁵Ac |
| ²²⁵Ac—C-28 | Lu—C-28 | ²²⁵Ac |
| ²²⁵Ac—C-26 | Lu—C-26 | ²²⁵Ac |
| ²²⁵Ac—C-30 | Lu—C-30 | ²²⁵Ac |
| ²²⁵Ac—C-31 | Lu—C-31 | ²²⁵Ac |
| ²²⁵Ac—C-32 | Lu—C-32 | ²²⁵Ac |
| ²²⁵Ac—C-33 | Lu—C-33 | ²²⁵Ac |
| ²²⁵Ac—C-34 | Lu—C-34 | ²²⁵Ac |
| ²²⁵Ac—C-35 | Lu—C-35 | ²²⁵Ac |
| ²²⁵Ac—C-44 | Lu—C-44 | ²²⁵Ac |
| ²²⁵Ac—C-45 | Lu—C-45 | ²²⁵Ac |
| ²²⁵Ac—C-41 | Lu—C-41 | ²²⁵Ac |
| ²²⁵Ac—C-42 | Lu—C-42 | ²²⁵Ac |
| ²²⁵Ac—C-64 | Lu—C-64 | ²²⁵Ac |
| ²²⁵Ac—C-62 | Lu—C-62 | ²²⁵Ac |
| ²²⁵Ac—C-72 | Lu—C-72 | ²²⁵Ac |
| ²²⁵Ac—C-63 | Lu—C-63 | ²²⁵Ac |

The molecular weight of the described peptide can vary. In some embodiments, the peptide has a molecular weight of about 0.1 to about 25 kDa. In some embodiments, the peptide has a molecular weight of about 0.2 to about 20 kDa, about 0.5 to about 15 kDa, about 0.75 to about 10 kDa, about 0.5 to about 10 kDa, about 0.5 to about 5 kDa, about 0.5 to about 2.5 kDa, about 0.5 to about 2 kDa, about 0.5 to about 1.5 kDa, about 0.5 to about 1 kDa, about 1 to about 10 kDa, about 1 to about 5 kDa, about 1 to about 2.5 kDa, about 1 to about 2 kDa, about 1 to about 1.5 kDa, about 1 to about 1.25 kDa, or about 0.5 to about 1.25 kDa. In some embodiments, the peptide has a molecular weight of about 0.5 to 5 kDa. In some embodiments, the peptide has a molecular weight of about 0.5 to 2 kDa. In some embodiments, the peptide has a molecular weight of about 0.75 to 1.75 kDa. In some embodiments, the peptide has a molecular weight of about 1 to 1.5 kDa. In some embodiments, the peptide is monocyclic.

A peptide described herein can be cyclized (i.e., macrocyclized). Cyclization can be achieved less ideally via a single disulfide bond, or more ideally via a peptide bond, alkyl bond, alkenyl bond, ester bond, thioester bond, ether bond, thioether bond, phosphate ether bond, azo bond, C—S—C bond, C—N—C bond, C═N—C bond, C═N—O bond, amide bond, lactam bridge, carbamoyl bond, urea bond, thiourea bond, amine bond, thioamide bond, or the like, but not limited to them. In some embodiments, the peptide is a cyclic peptide that is cyclized by a peptide bond, alkyl bond, alkenyl bond, ester bond, thioester bond, ether bond, thioether bond, phosphate ether bond, azo bond, C—N—C bond, C═N—C bond, C═N—O bond, amide bond, lactam bridge, carbamoyl bond, urea bond, thiourea bond, amine bond, or thioamide bond. In some embodiments, the cyclic peptide is cyclized by a thioether bond. In some embodiments, the cyclic peptide is cyclized via an oxime cyclization reaction. A cyclization of a peptide sometimes stabilizes the peptide structure and thereby enhance affinity for a target. The cyclization can occur between the N- and C-terminus, or it can occur between a terminal amino acid and a non-terminal amino acid. In some embodiments, the cyclization occurs between two non-terminal amino acids. In some embodiments, the peptide is cyclized via oxime cyclization. In some embodiments, the peptide is cyclized between cysteine and haloacyl. In some embodiments, the peptide comprises a haloacetyl group (e.g., chloroacetyl or bromoacetyl) at the N-terminus. In some embodiments, the peptide comprises a haloacetyl group (e.g., chloroacetyl or bromoacetyl) at the C-terminus. In some embodiments, the peptide comprises a Cy s at the C-terminus. In some embodiments, the peptide comprises a Cys at the N-terminus. In some embodiments, the cyclization occurs via a thioether bond bet-ween Cys and a haloacetyl group. In some embodiments, the cyclization occurs between the N-terminus and the C-terminus of the peptide.

As amino acids for macrocyclization, for example, an amino acid having the following functional group A and an amino acid having a corresponding functional group B can be used (see Table 4A). Either the functional group A or the functional group B may be placed on the N-terminal side. The amino acid having the functional group A and the amino acid having the functional group B can each be an N-terminal amino acid or C-terminal amino acid or a non-terminal amino acid. In some embodiments, an amino acid having the functional group A is placed at the N-terminus. In some embodiments, an amino acid having the functional group A is placed at the C-terminus. In some embodiments, an amino acid having the functional group A is placed at a non-terminal amino acid. In some embodiments, an amino acid having the functional group B is placed at the N-terminus. In some embodiments, an amino acid having the functional group B is placed at the C-terminus. In some embodiments, an amino acid having the functional group B is placed at a non-terminal amino acid.

TABLE 4A
Functional groups for cyclization

| | Functional group A | | Functional group B | |
|---|---|---|---|---|
| (I) | 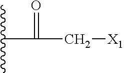 | (I-A) | 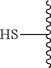 [SMILES: *S] | (I-B) |
| | X₁ is a halogen such as Cl, Br or I | | | |
| (II) | 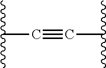 [SMILES: *C#C*] | (II-A) | 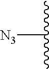 [SMILES: *N=[N+]=[N-]] | (II-B) |
| (III) | 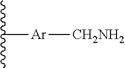 | (III-A) | 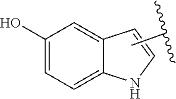 | (III-B) |
| | Ar is substituted or unsubstituted | | | |
| | aryl or heteroaryl | | | |
| (IV) | 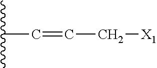 | (IV-A) | 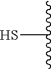 [SMILES: *S] | (IV-B) |
| | X₁ is a halogen such as Cl, Br or I | (V-A) | | (V-B) |
| (V) | 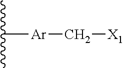 | | 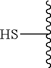 [SMILES: *S] | |
| | X₁ is a halogen such as Cl, Br or I; Ar is | | | |
| | substituted or unsubstituted aryl or heteroaryl | | | |
| (VI) | 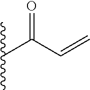 [SMILES: *C(=O)C=C] | (VI-A) | 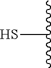 [SMILES: *S] | (VI-B) |
| (VII) | 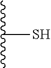 [SMILES: *S] | (VII-A) | 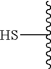 [SMILES: *S] | (VII-B) |
| (VIII) | 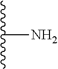 [SMILES: *N] | (VIII-A) | 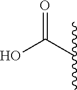 [SMILES: *C(=O)O] | (VIII-B) |
| (IX) | 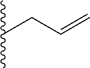 [SMILES: *CC=C] | (IX-A) | 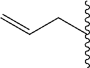 [SMILES: *CC=C] | (IX-B) |

In some embodiments, the amino acid (I-A) in Table 4A can be, for example, a chloroacetylated amino acid. Exemplary chloroacetylated amino acids include N-chloroacetyl-L-alanine, N-chloroacetyl-L-phenylalanine, N-chloroacetyl-L-tyrosine, N-chloroacetyl-L-tryptophan, N-3-(2-chloroacetamido)benzoyl-L-phenylalanine, N-3-(2-chloroacetamido)benzoyl-L-tyrosine, N-3-(2-chloroacetamido)benzoyl-L-tryptophan, β-N-chloroacetyl-L-diaminopropanoic acid, γ-N-chloroacetyl-L-diaminobutyric acid, σ-N-chloroacetyl-L-omithine, ε-N-chloroacetyl-L-lysine, N-3-chloromethylbenzyl-L-tyrosine, and N-3-chloromethylbenzyl-L-tryptophane and D-amino acid derivatives corresponding thereto (for example, N-Chloroacetyl-D-alanine, N-Chloroacetyl-D-phenylalanine, N-Chloroacetyl-D-tyrosine, and N-Chloroacetyl-D-tryptophan).

Examples of the amino acid (I-B) include, but are not limited to, cysteine, homocysteine, mercaptonorvaline, mercaptonorleucine, 2-amino-7-mercaptoheptanoyl acid, 2-amino-8-mercaptooctanoic acid, and amino acids obtained by protecting the SH group of these amino acids and then eliminating the protecting group, and D-amino acid derivatives corresponding thereto.

The cyclization method can be carried out, for example, according to the method described in Kawakami, T. et al., Nature Chemical Biology 5, 888-890 (2009); Yamagishi, Y. et al., ChemBioChem 10, 1469-1472 (2009); Sako, Y. et al. Journal of American Chemical Society 130, 7932-7934 (2008); or WO2008/117833.

In some embodiments, for example, the amino acid (II-A) is selected from propargylglycine, homopropargylglycine, 2-amino-6-heptynoic acid, 2-amino-7-octynoic acid, and 2-amino-8-nonynoic acid can be used. In addition, 4-pentynoylated or 5-hexynoylated amino acids can also be used. Examples of the 4-pentynoylated amino acids include N-(4-pentenoyl)-L-alanine, N-(4-pentenoyl)-L-phenylalanine, N-(4-pentenoyl)-L-tyrosine, N-(4-pentenoyl)-L-tryptophan, N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-(4-pentenoyl)-L-diaminopropanoic acid, γ-N-(4-pentenoyl)-L-diaminobutyric acid, γ-N-(4-pentenoyl)-L-ornithine, and ε-N-(4-pentenoyl)-L-lysine, and D-amino acid derivatives corresponding thereto.

In some embodiments, for example, the amino acid (II-B) is selected from azidoalanine, 2-amino-4-azidobutanoic acid, azidoptonorvaline, azidonorleucine, 2-amino-7-azidoheptanoic acid, and 2-amino-8-azidooctanoic acid can be used. In addition, azidoacetylated or 3-azidopentanoylated amino acids can also be used. Examples of the azidoacetylated amino acids include N-azidoacetyl-L-alanine, N-azidoacetyl-L-phenylalanine, N-azidoacetyl-L-tyrosine. N-azidoacetyl-L-tryptophan. N-3-(4-pentynoylamido)benzoyl-L-phenylalanine, N-3-(4-pentynoylamido)benzoyl-L-tyrosine, N-3-(4-pentynoylamido)benzoyl-L-tryptophan, β-N-azidoacetyl-L-diaminopropanoic acid, γ-N-azidoacetyl-L-diaminobutyric acid, α-N-azidoacetyl-L-ornithine, and ε-N-azidoacetyl-L-lysine, and D-amino acid derivatives corresponding thereto.

The cyclization method can be performed, for example, according to the method described in Sako, Y. et al., Journal of American Chemical Society 130, 7932-7934 (2008) or WO2008/117833.

Examples of amino acid (III-A) include, but are not limited to. N-(4-aminomethyl-benzoyl)-phenylalanine (AMBF) and 4-3-aminomethyltyrosine.

Examples of the amino acid (III-B) include, but are not limited to, 5-hydroxytryptophan (WoH). The cyclization method can be performed, for example, according to the method described in Yamagishi, Y. et al., ChemBioChem 10, 1469-1472 (2009) or WO2008/l 17833.

Examples of the amino acid (IV-A) include, but are not limited to, 2-amino-6-chloro-hexynoic acid, 2-amino-7-chloro-heptynoic acid, and 2-amino-8-chloro-octynoic acid.

Examples of the amino acid (IV-B) include, but are not limited to, cysteine, homocysteine, mercaptonorvaline, mercaptonorleucine, 2-amino-7-mercaptoheptanoic acid, and 2-amino-8-mercaptooctanoic acid, amino acids obtained by protecting the SH group of these amino acids and then eliminating the protecting group, and D-amino acid derivatives corresponding thereto. The cyclization method can be performed, for example, according to the method described in WO2012/074129.

Examples of the amino acid (V-A) include, but are not limited to, N-3-chloromethylbenzoyl-L-phenylalanine, N-3-chloromethylbenzoyl-L-tyrosine, and N-3-chloromethylbenzoyl-L-tryptophane.

Examples of the amino acid (V-B) include, but are not limited to, cysteine, homocysteine, mercaptonorvaline, mercaptonorleucine, 2-amino-7-mercaptoheptanoic acid, and 2-amino-8-mercaptooctanoic acid, and amino acids obtained by protecting the SH group of these amino acids and then eliminating the protecting group, and D-amino acid derivatives corresponding thereto.

The amino acids I-A to V-A and I-B to V-B can be introduced into the peptide in a known manner by chemical synthesis or translation and synthesis described herein. In some embodiments, the cyclization reaction comprises forming a thioether bond using an amino acid comprising a sulfanyl group, e.g., cysteine, homocysteine, mercaptonorvaline, mercaptovaline, mercaptonorleucine, 2-amino-7-mercaptoheptanoic acid, and 2-amino-8-mercaptooctanoic acid.

A peptide described herein can comprise one or more negatively charged amino acids and/or one or more positively charged amino acids. Positively charged amino acids include, for example, lysine, arginine, histidine, and amino acids that contain additional amine groups. Positively charged amino acids can comprise a heteroaryl substitution such as pyridine, imidazole, pyrazole, or triazole that has one or more ring nitrogen atoms. Negatively charged amino acids include, for example, amino acids that contain an additional carboxylic acid group such as glutamic acid or the like.

In some embodiments, a cyclic peptide of Formula (I), Formula (I-1). Formula (I-2). Formula (I-3), Formula (I-4), or Formula (I-5) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. In some embodiments, a cyclic peptide of Formula (I). Formula (I-1), Formula (I-2), Formula (I-3), Formula (I-4), or Formula (I-5) has a net charge of at most −4. In some embodiments, the cyclic peptide has a net charge of −4. In some embodiments, a cyclic peptide of Formula (I). Formula (I-1), Formula (I-2), Formula (I-3), Formula (I-4), or Formula (I-5) has a net charge of at least +2. In some embodiments, the cyclic peptide has a net charge of +2. In some embodiments, the cyclic peptide has a net charge of +3. The net charge can be determined by aggregating the charge of each of the X1 to X12 amino acids (or each of the amino acid in the peptide). For example, aspartic acid (D) and glutamic acid (E) each has a charge of −1, lysine (K), arginine (R) and histidine (H) each has a charge of +1, and the rest of the canonical amino acids each has a charge of 0.

In some embodiments, a cyclic peptide of formula (I) has a net charge of −3 to +1. In some embodiments, the cyclic peptide has a net charge of −3. In some embodiments, the cyclic peptide has a net charge of −2. In some embodiments, the cyclic peptide has a net charge of −1. In some embodiments, the cyclic peptide has a net charge of 0. In some embodiments, the cyclic peptide has a net charge of +1. The net charge can be determined by aggregating the charge of each of the amino acids of the cyclic peptide.

In some embodiments, a cyclic peptide of Formula (I), Formula (I-1), Formula (I-2), Formula (I-3), Formula (I-4), or Formula (I-5) does not contain any S—S bond.

In some embodiments, a peptide of the present disclosure can be cyclized by forming a group as illustrated in Table 4B.

TABLE 4B
Ring Closing Groups (m and n are independently
0 or an integer from 1 to 6.)

—C(═O)—CH₂—
—C(═O)—CH₂—S—
—C(═O)—CH₂—S—CH₂—
—C(═O)—CH₂—S—CH₂—CH₂—
—(CH₂)ₘ—NH—CO—(CH₂)ₙ—
—(CH₂)ₘ—CO—NH—(CH₂)ₙ—
—(CH₂)ₘ—S—(CH₂)ₙ—
—(CH₂)ₘ—CH═CH—(CH₂)n—
—(CH₂)ₘ—NH—(CH₂)n—
—(CH₂)ₘ—S—CH₂-benzene-CH₂—S—(CH₂)ₙ—
—(CH₂)ₘ-triazole-(CH₂)ₙ—
—(CH₂)ₘ-succinimide-S—(CH₂)ₙ—
—C(═O)—CH₂—NH—CH₂—
—C(═O)—CH₂—O—CH₂—
—C(═O)—CH₂—CH₂—S—
—(CH₂)ₘ—S—S—(CH₂)ₙ—
—(CH₂)ₘ—C(═O)—NH—(CH₂)ₙ—
—(CH₂)ₘ—CH₂—CH₂—(CH₂)ₙ—

In some embodiments, m is 0 and n is 0. In some embodiments, m is 0. In some embodiments, in is 1. In some embodiments, m is 2. In some embodiments, m is 3. In some embodiments, in is 4. In some embodiments, m is 5. In some embodiments, in is 6. In some embodiments, n is 0. In some embodiments, n is 1. In some embodiments, n is 2. In some embodiments, n is 3. In some embodiments, n is 4. In some embodiments, n is 5. In some embodiments, n is 6.

In some embodiments, a peptide of the present disclosure, e.g., peptides of Formulae (I), can be cyclized by reacting a first functional group with a second functional group, see Table 4C. In some embodiments, the first functional group is located at the N-terminus. In some embodiments, the first functional group is located at a non-terminal amino acid. In some embodiments, the second functional group is located at the C-terminus. In some embodiments, the second functional group is located at a non-terminal amino acid.

TABLE 4C
Formation of Ring Closing Groups

| First Functional group | Second Functional group (or amino acid) (e.g., |
|---|---|
| (e.g., at N-terminus) | at C-terminus or at a non-terminal amino acid) |
| —C(═O)—CH₂Cl | Cysteine, homocysteine, lysine, homolysine, |
| | ornithine, diaminobutric acid, serine, homoserine, |
| | threonine, homothreonine |
| —(CH₂)ₙ—NH₂ | Aspartic acid, glutamic acid, homoglutamic acid |
| —(CH₂)ₙ—CO₂H | Lysine, homolysine, ornithine, diaminobutric |
| | acid, |
| —(CH₂)ₙ—Br | Cysteine, homocysteine |
| —(CH₂)ₙ—CH═CH₂ | Allyl-glycine, homoallyl-glycine |
| —(CH₂)ₙ—NH₂ | Aspartate-4-semialdehyde, glutamate-5- |
| | semialdehyde |
| —(CH₂)ₙ—SH | Cysteine, homocysteine conjugated with 1,2- or |
| | 1,3- or 1,4-bis-(bromomethyl)benzene |
| —(CH₂)ₙ-alkyne | XaaC is an amino acid with a side chain with |
| | azide |
| —(CH₂)ₙ—N₃ | Alkynyl-glycine, homoalkynyl-glycine |
| —(CH₂)ₙ-maleimide | Cysteine, homocysteine |

In some embodiments, a conjugate comprising any one of peptide of Table 1 may further comprise amino acid residues at the N and/or C terminus of the peptide, which is not part of the cyclic structure. In some embodiments, the conjugate further comprises a metal chelator and optionally a linker. In some embodiments, the conjugate further comprises a radionuclide such as Ac-225 or Lutetium-177. In other embodiments, the conjugate comprises a covalently bound radionuclide and optionally a linker. In some further embodiments, the conjugate comprises a covalently bound radionuclide such as ¹³¹I.

A peptide described herein can be a peptide mimetic. For example, the peptide can comprise non-peptide bonds and it can comprise one or more unnatural amino acids. Unless stated otherwise, each of the amino acid in a peptide described herein (except the natural amino acid glycine) can independently be in its D or L form. Both D and L forms are encompassed by the present disclosure.

In the present disclosure, the term amino acid embraces derivatives of amino acids. The derivatives include, for example, amino acids obtained by modifying a natural amino acid constituting a protein produced by cellular DNA-encoded biological matter. Examples of such non-natural amino acids include hydroxyproline and hydroxylysine, which are amino acids having a hydroxyl group introduced therein, and diaminopropionic acid, which is an amino acid having an amino group introduced therein.

A peptide described herein can comprise an N-substituted amino acid. In some embodiments, the N-substituted amino acid is a derivative of tryptophan, phenylalanine, tyrosine, arginine, histidine, isoleucine, leucine, lysine, or valine. In some embodiments, the N-substitution is an N-alkyl, such as N-methyl and N-ethyl. In some embodiments, the N-substitution is N-methyl. In some embodiments, the N-substitution is an N-aryl, such as N-phenyl or N-biphenyl. In some embodiments, the N-substitution is an N-heteroaryl such as N-pyridyl. In some embodiments, the N-substituted amino acid is at the N-terminus of the peptide. In some embodiments, the N-substituted amino acid is a non-terminal amino acid.

In some embodiments, peptides described herein comprise one or more amino acids in Tables 5A to 5G

TABLE 5A
Exemplary Amino Acids at N or C-terminus

| | |
|---|---|
| N-Chloroacetyl-L-alanine | Acetyl-L-alanine |
| N-Chloroacetyl-L-phenylalanine | Acetyl-L-phenylalanine |
| N-Chloroacetyl-L-phenylalanine | Acetyl-L-tyrosine |
| N-Chloroacetyl-L-tyrosine | Acetyl-L-tryptophan |
| N-Chloroacetyl-L-tryptophan | Acetyl-D-alanine |
| N-Chloroacetyl-D-alanine | Acetyl-D-phenylalanine |
| N-Chloroacetyl-D-phenylalanine | Acetyl-D-tyrosine |
| N-Chloroacetyl-D-tyrosine | Acetyl-D-tryptophan |
| N-Chloroacetyl-D-tryptophan | N-3-chloromethylbenzoyl-L-tyrosine |
| | N-3-chloromethylbenzoyl-L-tryptophan |

TABLE 5B
Exemplary Amino Acids That Crosslink With A Peptide

Nγ-(2-chloroacetyl)-α,γ-diaminobutylic acid
Nγ-(2-chloroacetyl)-α,γ-diaminopropanoic acid

TABLE 5C
D-amino Acids

D-Serine
D-Phenylalanine
D-Tyrosine
D-Tryptophan

TABLE 5D
Exemplary N-alkylamino Acids

N-alkyl-Glycine
N-alkyl-Alanine
N-alkyl-Phenylalanine
N-alkyl-Tyrosine
N-alkyl-Serine
N-alkyl-Histidine
N-alkyl-Tryptophan

Exemplary alkyl groups for Table 5D include methyl, ethyl, and propyl groups.

TABLE 5E
Exemplary Peptoid Blocks

N-ethyl-Glycine
N-n-propyl-Glycine
N-n-butyl-Glycine
N-n-pentyl-Glycine
N-n-hexyl-Glycine
N-n-heptyl-Glycine
N-n-octyl-Glycine
N-isopentyl-Glycine
N-(2-phenylethyl)-Glycine
N-(3-phenylpropyl)-Glycine
N-[2-(p-hydroxyphenyl)ethyl]-Glycine

TABLE 5F
Exemplary Unnatural Amino Acids

p-biphenylalanine
p-trifluoromethylphenylalanine
p-azidophenylalanine
p-biotinyl-aminophenylalanine
e-N-Biotinyl-lysine
e-N-Acetyl-lysine
L-Citrulline
L-5-Hydroxytryptphan
L-1,2,3,4,-Tetrahydroisoquinoline-3-carboxylic acid
Aminoisobutyric acid
N-methyl-aminoisobutyric acid
N-methyl-Phenylglycine

Amino acids used in the disclosed peptides can be substituted with similar amino acids. In some embodiments, an amino acid can be substituted with another amino acid with similar hydrophobicity. In some embodiments, an amino acid can be substituted with another amino acid with similar hydrophilicity. In some embodiments, an amino acid can be substituted with another amino acid with similar size. In some embodiments, an amino acid can be substituted with another amino acid with similar charge. In some embodiment, an amino acid can be substituted with another amino acid with a similar functional group. In some embodiments, an amino acid can be substituted with another amino acid with the same functional group.

In some embodiments, an amino acid described herein can be replaced with a variant thereof. Examples of an amino acid substitution or variant include derivatives having an amine, amide, ester, or carboxyl group as the C-terminus and/or N-terminus thereof. Additional examples of amino acid/peptide variants include those obtained by modification such as phosphorylation, alkylation (e.g., methylation), acetylation, adenylylation, ADP-ribosylation, or glycosylation and fused protein obtained by fusion with another peptide or protein. These variants can be prepared by those skilled in the art in a known manner or a method based thereon. An amino acid variant further encompasses the amino acids that have the same functional groups but with different lengths of the side chain (e.g., LysAc vs. OrnAc and cysteine vs. homocysteine). An amino acid variant further encompasses amino acids with a different aromatic moiety compared to the canonical amino acid (e.g., the indole in tryptophan vs the 7-azaindole in 7-AzaTrp; the phenyl in phenylalanine vs the pyridine in 4Py). An amino acid variant further encompasses amino acids with optional substituents. i.e., optionally substituted amino acid. In some embodiments, the optionally substituted amino acid is optionally substituted with one or more substituents independently selected from halogen, hydroxyl, cyano, amino, amide, nitro, ureido, C₁-C₆ alkyl, C₁-C₆ alkoxy, C₆-C₁₀ aryl, C₃-C₆ cycloalkyl, 6-10 membered heterocycloalkyl, and 6-10 membered heteroaryl. In some embodiments, the optionally substituted amino acid is optionally substituted with one or more substituents independently selected from halogen, —CN, —NH₂, —NH(alkyl), —N(alkyl)₂, oxo, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), SF⁵, —R^{b}—OR^{a}, —R^{b}—OC(O)—R, —R^{b}—OC(O)—OR^{a}, —R^{b}—OC(O)—N(R^{a})₂, —R^{b}—N(R^{a})₂, —R^{b}—C(O)R², —R^{b}—C(O) OR, —R^{b}, —C(O)N(R^{a})₂, —R^{b}—O—R^{c}—C(O)N(R^{a})₂, —R^{a}—N(R^{a})C(O)OR^{a}, —R^{b}—N(R^{a})C(O)R^{a}, —R^{b}—N(R^{a})S(O)₂R^{a}(where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R)₂, —R^{c}—N(R)₂, —R^{b}—C(O)R^{a}, —R^{d}—C(O)OR^{a}, —R^{b}—C(O)N(R^{a})₂, —R—O—R^{a}—C(O)N(R^{a})₂, —R^{b}—N(R^{a})C(O)OR^{a}, —R^{b}—N(R^{a})C(O)R^{a}, —R^{b}—N(R^{a})S(O)R (where t is 1 or 2), —R^{b}—S(O)ₜR^{a} (where t is 1 or 2), —R^{b}—S(O)ₜOR^{a} (where t is 1 or 2) and —R^{b}—S(O)₁N(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{c}—OC(O)—R^{a}, —R^{b}—OC(O)—OR^{a}, —R^{b}—OC(O)—N(R^{a})₂, —R^{c}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)₁R^{a} (where t is 1 or 2), —R^{b}—S(O)ₜOR^{a} (where t is 1 or 2) and —R^{b}—S(O)₁N(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

In some embodiments, a variant of an amino acid is selected from amino acids having one, two or three substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, —S(═O)₂NH₂, —S(═O)₂NH(C₁-C₃alkyl), —S(═O)₂N(C₁-C₃alkyl)₂. C₁-C₆ alkyl, C₁-C₆ heteroalkyl, C₁-C₆ alkoxy, C₆-C₁₀ aryl, C₃-C₆ cycloalkyl, 6-10 membered heterocycloalkyl, and 6-10 membered heteroaryl. In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, oxo, —OH, —CO₂H, —CO₂—C₁-C₃alkyl, —C(═O)NH₂, —C(═O)NH(C₁-C₃alkyl), —C(═O)N(C₁-C₃alkyl)₂, and C₁-C₆ alkyl. In some embodiments, the variant is selected from amino acids having one or two substituents based on the amino acid, and wherein the substituents are independently selected from halogen, —CN, —NH₂, —NH(C₁-C₃alkyl), —N(C₁-C₃alkyl)₂, and C₁-C₆ alkyl. In some embodiments, the variant is selected from amino acids having one or two substituents based on the amino acid, and wherein the substituents are independently selected from C₁-C₆ alkyl.

In some embodiments, a variant of an amino acid is selected from amino acids that have the similar hydrophilicity or hydrophobicity compared to the amino acid. Thus, in some embodiments, a positively charged amino acid can be a variant of another positively charged amino acid. In some embodiments, a negatively charged amino acid can be a variant of another negatively charged amino acid. In some embodiments, a zwitterionic amino acid can be a variant of another zwitterionic amino acid.

In some embodiments, a hydrophilic amino acid has an electrically charged side chain. In some embodiments, a hydrophilic amino acid has a positive charge. In some embodiments, a hydrophilic amino acid has a negative charge. In some embodiments, a hydrophilic amino acid is zwitterionic (e.g., KCOpipzaa). In some embodiments, a hydrophilic amino acid comprises a —OH, COOH, —NH— or NH₂ moiety. In some embodiments, a hydrophilic amino acid comprises —OH, —C(O)OH, —NHC(═NH)NH₂, —NHC(O)NH₂, —C(O)NH₂, or —NHC(O)CH₃. In some embodiments, a hydrophilic amino acid comprises a side chain of C₁-C₆hydroxyalkyl, C₁-C₆aminoalkyl, —C₀₋₆ alkylene-NH—C(═NH)—NH₂, —C₀₋₆ alkylene-CO—NH₂, —C₀₋₆ alkylene-COOH, or —NH—CO—C₁₋₆ alkyl.

In some embodiments, a hydrophobic amino acid is not charged. In some embodiments, a hydrophobic amino acid contains at least 2 contiguous carbon atoms. In some embodiments, a hydrophobic amino acid comprises at least 3 contiguous carbon atoms, either linear or branched. In some embodiments, a hydrophobic amino acid comprises at least 4 contiguous carbon atoms, either linear or branched. In some embodiments, a hydrophobic amino acid comprises at least 5 contiguous carbon atoms, either linear or branched. In some embodiments, a hydrophobic amino acid comprises an ethylene moiety in the side chain. In some embodiments, a hydrophobic amino acid comprises a propylene moiety in the side chain. In some embodiments, a hydrophobic amino acid comprises a butylene moiety in the side chain. In some embodiments, a hydrophobic amino acid comprises phenyl moiety. In some embodiments, a hydrophobic amino acid comprises a heteroaryl moiety. In some embodiments, a hydrophobic amino acid is Trp, Tyr, Phe, or derivatives thereof.

In some embodiments, a variant of an amino acid is selected from amino acids that have the same functional group as the amino acid, and wherein the variant has a different length of a side chain compared to the amino acid. In some embodiments, a variant of an amino acid is selected from amino acids that have the same charge compared to the amino acid. In some embodiments, a variant of an amino acid is selected from amino acids that have the same polarity compared to the amino acid. In some embodiments, an amino acid comprising an aromatic group can be a variant of another amino acid having an aromatic group. In some embodiments, an amino acid comprising a phenyl can be a variant of another amino acid having a phenyl. In some embodiments, an amino acid comprising a heteroaryl can be a variant of another amino acid having a heteroaryl. Amino acids having an aromatic group include, but are not limited to, F, W. F4G, 4Py 0.3Py, F4COO, F3G, 3Py6COO, 4Py2NH2, 3Py5COO, F3COO, 3Py6NHAc, F4C, F40Me, Nal2, F3aao, F4aa, F4aao, 3Py6NHaa, F3CON, F4F, F40Et, F4Me, F4CON, F4CONPEG4Me, F30Me, YaeCOpipzaa. F4aaopipzaa, 4Pdo, 3P,6CON, F4amCOpipzaa, F4u, F4CONdMe, F4 ms, F34dOMe. F3C, F3CONdMe, 5inda, F3aa, 3Py6NHae, 3Py6OMe, MeNal2, MeNal27N, MeF34diox, MeF34dOMe, MeF4T, MeW7N, MeF3C₄Me, MeF3Me4C, Bph, 3Py6Ph. F41Me4Pyz, F43Pyz, F44Pyz, F41Pyz, F41Me3Pyz, F41Et4Pyz, F41MeOe4Pyz, F41MeOp4Pyz, F44thp, F4Ac4Pip, PhNva, PhNIe, Yph, Ybn, F4tb, F4OPr, Yae, Me3Py, W5N and MeWIMe.

In some embodiments, an amino acid comprising a cycloalkyl group can be a variant of another amino acid having a cycloalkyl group. In some embodiments, an amino acid comprising a heterocycloalkyl group can be a variant of another amino acid having a heterocycloalkyl group.

In some embodiments, a variant of an amino acid is selected from amino acids that have similar polarity and/or charge with the amino acid. For example, in some embodiments, a polar, uncharged amino acid can be a variant of another polar, uncharged amino acid (e.g., Hgn, Q, S, T, Qglucamine),

In some embodiments, a variant of an amino acid has the same number of hydrogen donor as the amino acid. In some embodiments, a variant of an amino acid has the same number of hydrogen acceptor as the amino acid.

In some embodiments, the variant has a molecular weight that does not vary for more than 14, 28, 30, 45 or 60 g/mol compared to the amino acid. In some embodiments, the variant has a molecular weight that does not vary for more than 14 g/mol compared to the amino acid. In some embodiments, the variant has a molecular weight that does not vary for more than 50 g/mol compared to the amino acid. In some embodiments, the variant has a molecular weight that does not vary for more than 28 g/mol compared to the amino acid.

An amino acid variant further encompasses amino acids wherein a functional group is substituted with another functional group having similar properties, e.g., a cysteine can be substituted with a homocysteine. In some embodiments, an aryl functional group can be substituted with an aryl or heteroaryl group. In some embodiments, a heteroaryl functional group can be substituted with an aryl or heteroaryl group. In some embodiments, an amino functional group can be substituted with an NH(alkyl) group.

As used herein, the expression “conservative amino acid substitution” refers to a substitution of functionally equivalent or similar amino acids. A conservative amino acid substitution in a peptide brings about a static change to the amino acid sequence of the peptide. For example, one or two or more amino acids having similar polarity act functionally equivalent to each other and bring about a static change in the amino acid sequence of the peptide. In general, a substitution within a certain group may be considered conservative regarding structure and function. However, as is clear to a person having ordinary skill in the art, the role played by a defined amino acid residue may be determined by its implication in the three-dimensional structure of the molecule containing the amino acid. For example, a cysteine residue in an oxidized-type (disulfide) form may have a lower polarity than that of a reduced-type (thiol) form. The long aliphatic part of the arginine side chain may constitute structurally and functionally important features. Furthermore, the side chain (tryptophan, tyrosine, phenylalanine) including an aromatic ring may contribute to ion-aromatic interaction or cation-pi interaction. In such a case, even if the amino acids having these side chains are substituted for amino acids belonging to the acidic or non-polar groups, they may be structurally and functionally conservative. There is a possibility that residues such as proline, glycine, cysteine (disulfide foam) have a direct effect on the three-dimensional structure of the main chain and often may not be substituted without structural distortion.

Conservative amino acid substitution, as shown below, includes specific substitution based on the similarity of side chains (for example, substitutions are described in Lehninger, Biochemistry, Revised 2nd Edition, published in 1975, pp. 73 to 75: L. Lehninger, Biochemistry, 2nd edition. pp. 73 to 75, Worth Publisher, New York (1975)), incorporated herein by reference, and typical substitution.

Hydrophobic amino acids include amino acids that exhibit hydrophobicity, including alanine (also referred to as “Ala” or simply “A”), glycine (also referred to as “Gly” or simply “G”), valine (also referred to as “Val” or simply “V”), leucine (also referred to as “Leu” or simply “L”), isoleucine (also referred to as “Ile” or simply “I”), proline (also referred to as “Pro” or simply “P”), phenylalanine (also referred to as “Phe” or simply “F”), tryptophan (also referred to as Trp” or simply “W”), tyrosine (also referred to as “Tyr” or simply “Y”), and methionine (also referred to as “Met” or simply “M”).

Exemplary hydrophobic amino acids may be further divided into the following groups:
Aliphatic amino acids: Amino acids having a fatty acid or hydrogen in the side chain, including e.g., Ala, Gly, Val, Ile. and Leu.
Aliphatic/branched-chain amino acids: Amino acids having a branched fatty acid in the side chain, including e.g., Val, Ile, and Len.
Aromatic amino acids: Amino acids having an aromatic ring in the side chain, including e.g., Trp, Tyr, and Phe.

In some embodiments, a hydrophobic amino acid has a C1-C8 alkyl, cycloalkyl, or heterocycloalkyl, wherein the alkyl, cycloalkyl, and heterocycloalkyl are each independently, optionally substituted. In some embodiments, a hydrophobic amino acid has a C1-C8 alkyl, cycloalkyl, or heterocycloalkyl, wherein the alkyl, cycloalkyl, and heterocycloalkyl are each unsubstituted. In some embodiments, a hydrophobic amino acid has a C₃-C₆ unsubstituted alkyl. In certain embodiments, a hydrophobic amino acid is I, Eva, alI, TMe, SMe, Gepr, Gcpe, Gthp, dMeS, TdMe, or Cbg.

Hydrophilic amino acids include amino acids that exhibit hydrophilicity, including e.g., serine (also referred to as “Ser” or simply “S”), threonine (also referred to as “Thr” or simply “T”), cysteine (also referred to as “Cys” or simply “C”), asparagine (also referred to as “Asn” or simply “N”), glutamine (also referred to as “Gln” or simply “Q”), aspartic acid (also referred to as “Asp” or simply “D”), glutamic acid (also referred to as “Glu” or simply “E”), Elysine (also referred to as “Lys” or simply “K”), arginine (also referred to as “Arg” or simply “R”), and histidine (also referred to as “His” or “H”).

Exemplary hydrophilic amino acids may be further divided into the following groups:
Acidic amino acids: Amino acids whose side chains exhibit acidity, including Asp and Glu.
Basic amino acids: Amino acids whose side chains exhibit basicity, including Lys, Arg, and His.
Neutral amino acids: Amino acids whose side chains exhibit neutrality, including Ser. Thr, Asn, Gin, and Cys.

Exemplary hydrophilic amino acids include, for example, D, Q, A, E, S, N, T, C, H, or a variant thereof (including D-amino acid such as da and variations such as KCOpipzetOH, Cit, COmeglumine, KCOpipzaa, A4paa, Hgl, F4COO, KCOpip4COO, KAc, Hgn, Har, DapCOpipzaa, Acpr, Cba, NleCOO, NIeOH, Atb, Ny a, Nle, DapAc, Abu, Nmm, Ndm, Ncit, Cit or the like). In some aspect, hydrophilic amino acid is optionally N-methylated such as MeD.

In some embodiments, a peptide described herein comprises an amino acid that affects the direction of the main chain, e.g., Gly and Pro. In some embodiments, a peptide described herein comprises a sulfur-containing amino acid, e.g., Cys and Met. In some embodiments, a peptide described herein comprises an amino acid that comprises an aromatic ring, which can be optionally substituted. Amino acids comprising an aromatic ring include, e.g., F (Phe; phenylalanine), Y (Tyr:tyrosine), W (Trp; tryptophan).

In some embodiments, W or a variant thereof can be W, an amino acid having a heteroatom in the indole ring of W in the side chain, an amino acid in which the hydrogen of NH in the indole ring of W is substituted, or an amino acids having a substituent in the benzene ring of W, or the like.

In some embodiments, F or a variant thereof can be F (phenylalanine), an amino acid comprising a phenyl, 6 membered heteroaryl (such as pyridine, pyrimidine, pyrazine), indazolyl, or naphthalyl in its side chain, wherein said phenyl, heteroaryl, indazolyl or naphthalyl is optionally substituted. In some embodiments, F or a variant thereof can be F (phenylalanine), an amino acid comprising a phenyl, naphthalyl, 6 membered heteroaryl, or bicyclic heteroaryl in its side chain, wherein said phenyl, heteroaryl, or naphthalyl is optionally substituted. In some embodiments, F or a variant thereof can be F (phenylalanine), an amino acid comprising a phenyl, pyridinyl, indazolyl, or naphthalyl in its side chain, wherein said phenyl, pyridinyl, indazolyl or naphthalyl is optionally substituted with one or more substituents each independently selected from halogen, —C1-3alkyl, —OH, —NH₂, —CN, —C(═O)OH, —C(═O)NH₂, —NHC(═O)CH₃, —C1-3alkylene-C(═O)OH, —C1-3alkylene-C(═O)NH₂, —O-C1-3alkylene-C(═O)OH, —O-C1-3alkylene-C(═O)NH₂, —C1-3alkylene-C(═O)—S-to 6-membered heterocycloalkylene-C1-3alkylene-C(═O)OH, —O-C1-3alkylene-C(═O)-5-to 6-membered heterocycloalkylene-C1-3alkylene-C(═O)OH, —C1-3alkylene-NIC(═O)-5-to 6-membered heterocycloalkylene-C1-3alkylene-C(═O)OH, —O-C1-3alkylene-NHC(═O)-5-to 6-membered heterocycloalkylene-C1-3alkylene-C(═O)OH, and —NH—C1-3alkylene-C(═C)OH, —NHC(═O)NH₂, —C₁₋₃alkylene-C(NH₂)—COOH, —NH—CO—CH₃, —NH—C₁₋₃alkylene-NH₂, —C(═O)—N(CH₂)₂, —S(═O)₂—CH₃, —C₁₋₃alkylene-NH—C(═O)-5-to 6-membered heterocycloalkylene-C₁₋₃alkylene-C(═O)OH, and —O—C₁₋₃alkylene-NH—C(═O)-5-to 6-membered heterocycloalkylene-C1-3alkylene-C(═O)OH, —O-phenyl, —O—C₁₋₃alkylene-phenyl, pyridinyl, imidazolyl, pyrazolyl, N—C₁₋₃alkylene pyrazolyl, N—C₁₋₃alkylene(—O—C₁₋₃alkyl) pyrazolyl, pyranyl, tetrahydropyranyl, piperidinyl, N—C₁₋₃alkylene-C(═O)-piperidinyl; or the like. In some aspect. F or a variant thereof is optionally N-methylated.

In some embodiments, F or a variant thereof can be F (phenylalanine), an amino acid wherein (i) the phenyl ring of F is substituted with 1 or 2 substituents each independently selected from —OH, —CN, —C₁₋₃ alkyl, such as —CH₃; (ii) a 6-membered heteroaryl ring optionally substituted by 1 or 2 substituents each independently selected from —OH, —CN, —C₁₋₃ alkyl, such as —CH₃; or (iii-1) having a heteroatom in the phenyl ring of F in the side chain: (iii-2) a derivative amino acid of F in which a 6-membered heteroaryl ring in the side chain is substituted; or the like. In some aspect, F or a variant thereof is optionally N-methylated.

In some embodiments, Y or a variant thereof can be Y, an amino acid having a hydroxyphenyl ring, wherein the hydrogen atom in hydroxyphenyl of Y or of the variant is optionally substituted. In some embodiments, Y or a variant thereof can be Y, an amino acid having a hydroxyphenyl ring, wherein the hydrogen atom in hydroxyphenyl of Y or of the variant is optionally substituted with one or more substituents selected from —C1-3alkyl, -C1-3alkylene-C(═O)OH, halogen, —OH, —C(═O)OH, —O—CH₃, —C₁₋₃alkylene-C(═O)-5-to 6-membered heterocycloalkylene-C₁₋₃alkylene-C(═O)OH, and —C₁₋₃alkylene-NHC(═O)-5-to 6-membered heterocycloalkylene-C₁₋₃alkylene-C(═O)OH—. In some embodiments, a variant of W or a variant thereof can be W, WIMe, or W7N or the like. In some embodiments, W or a variant thereof is optionally N-methylated (e.g., MeWIMe or MeW7N).

In certain embodiments, F or Y or a variants thereof is Y, F3G, 3Py6COO, 4Py2NH2, 3Py5COO, F3COO, 3Py6NHAc, F, F4C, F40Me, F4COO, Nal2, F3aao, F4aa, F4aao, 3Py6NHaa, 5Pdo, F3CON, F4F. F40Et, F4Me, F4CON, F4CONPEG4Me, F30Me, YaeCOpipzaa, F4aaopipzaa, 4Pdo, 3Py6CON, Atp, Cha4cH, Cha4tH, Cha4cOMe, A1mor, F4amCOpipzaa, F40Me, F4u, F4CONdMe, F4 ms. F34dOMe, F3C, F3CONdMe, 5Inda. F3aa, 3Py6Nhae, 3Py6OMe, F4amCOpipzaa, Bph, 3Py6Ph, F41Me4Pyz, F43Pyz. F44Pyz, F41Pyz, F41Me3Pyz, F41Et4Pyz, F41MeOe4Pyz, F41MeOp4Pyz, F44thp, F4Ac4pip, PhNva, PhNle, Yph, Ybn, F4tb, or F4oPr.

In some embodiments, a monocyclic aromatic amino acid can be an amino acid having a phenyl or monocyclic heteroaryl, each of which optionally substituted. In some embodiments, a monocyclic aromatic amino acid can be an amino acid having a phenyl or pyridinyl optionally substituted with one or more substituents each independently selected from halogen, -C1-3alkyl, and trifluoromethyl. In some embodiment, a bicyclic aromatic amino acid can be an amino acid having a naphthalyl or bicyclic heteroaryl, each of which optionally substituted. In some embodiment, a bicyclic aromatic amino acid can be an amino acid having a naphthalyl, quinolyl, or indazolyl optionally substituted with one or more substituents each independently selected from H or C1-3alkyl. In some embodiments, bicyclic aromatic acid can be W (tryptophan), or a variant thereof, or N-methylated thereof. In certain embodiments, a monocyclic aromatic amino acid is MeF3C3Me, MeF34dOMe, MeF4T, or M3FMe4C. In certain embodiments, a bicyclic aromatic amino acid is MeNal2, MeNal27N, MeF34diox, MeW1NMe, or MeW7N.

In some embodiments, an amino acid described herein is N-alkylated.

In some embodiments, an amino acid described herein is not N-alkylated (e.g., an amino acid with —H on the alpha-amino group).

Examples of the amino acids include natural protein L-amino acids, unnatural amino acids, and chemically synthesized compounds having properties known in the art as characteristics of an amino acid. Examples of the unnatural amino acids include, but not limited to. α,α-disubstituted amino acids (such as α-methylalanine). N-alkyl-α-amino acids, D-amino acids, β-amino acids, and α-hydroxy acids, each having a backbone structure different from that of natural amino acids; amino acids (such as norleucine and homohistidine) having a side-chain structure different from that of natural amino acids; amino acids (such as “homo” amino acids, homophenylalanine, and homohistidine) having extra methylene in the side chain thereof; and amino acids (such as cysteic acid) obtained by substituting a carboxylic acid functional amino group in the side chain thereof by a sulfonic acid group.

The peptides described herein can comprise one or more unnatural amino acids. Unnatural amino acids include, but are not limited to, (1) amino acids corresponding to an amino acid residue on a polypeptide subjected to modification after expression (ex, phosphorylated tyrosine, acetylated lysine, or farnesylated cysteine), (2) amino acids that cannot be used in expression on a ribosome but occur naturally, and (3) artificial amino acids that do not occur naturally (unnatural amino acids). Non-limiting examples of unnatural amino acids include: p-acetyl-L-phenylalanine, p-iodo-L-phenylalanine, p-methoxyphenylalanine, O-methyl-L-tyrosine, p-propargyloxyphenylalanine, p-propargyl-phenylalanine, L-3-(2-naphthyl)alanine, 3-methyl-phenylalanine. O-4-allyl-L-tyrosine, 4-propyl-L-tyrosine, tri-O-acetyl-GlcNAcp-serine, L-Dopa, fluorinated phenylalanine, isopropyl-L-phenylalanine, p-azido-L-phenylalanine, p-acyl-L-phenylalanine, p-benzoyl-L-phenylalanine, Boronophenylalanine, O-propargyltyrosine, L-phosphoserine, phosphonoserine, phosphonotyrosine, p-bromophenylalanine, selenocysteine, p-amino-L- phenylalanine, isopropyl-L-phenylalanine, and azido-lysine (AzK). In some embodiments, the unnatural amino acid is an unnatural analogue of a tyrosine amino acid; an unnatural analogue of a glutamine amino acid; an unnatural analogue of a phenylalanine amino acid; an unnatural analogue of an alanine amino acid; an unnatural analogue of a serine amino acid; an unnatural analogue of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid; or a combination thereof. In some embodiments, the unnatural amino acid is an amino acid with a photoactivatable cross-linker; a spin-labeled amino acid; a fluorescent amino acid; a metal binding amino acid: a metal-containing amino acid: a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group: a sugar substituted amino acid; a carbon-linked sugar-containing amino acid; a redox-active amino acid; an α-hydroxy containing acid; an amino thio acid; an α, α-disubstituted amino acid: a β-amino acid; a cyclic amino acid other than proline or histidine, or an aromatic amino acid other than phenylalanine, tyrosine or tryptophan.

Unnatural amino acids include, for example, N-alkyl amino acids in which a natural amino acid described above is N-alkylated, e.g., those modified with lower alkyl groups (for example, of C1 to C5, C1 to C3, and C1) in which the nitrogen forming a peptide bond is branched or not branched. Exemplary N-alkyl amino acids include, e.g., N-ethyl amino acid, N-butyl amino acid, and N-methyl amino acid. Also included are amino acids to which a functional group is further added to the side chain of a natural amino acid or substituted for another functional group (for example, an amino acid having a substitution or an addition in a part such as an arylene group, an alkylene group, or the like of the side chain; an amino acid wherein the arylene group or the alkyl group of the side chain has an increased C-number; an amino acid having a substitution in the aromatic ring of the side chain; a heterocyclic or condensed cyclic amino acid; or the like). Exemplary N-alkyl amino acids further include, e.g., N-alkyllysine and N-methyllysine.

In a non-limiting manner, unnatural amino acids include, but are not limited to N-methyl amino acids. F4G, 4Py, 3Py. Cit, KCOpipzaa. Eva. Ahp. F4COO, KCOpip4COO, Aib. Hpr. Sbu, MeDapCOpipzaa, Scbm, Scpe, HseBu, A4paa, Spent, Hgl, Hsecpe, Hgn, DapCOpipzaa, MeD, CmG, Medd, HseEt, HseiPr, CrmG, CeG, CrpG, MeIHgL MeCit, F3G, 3Py6COO, 4Py2NH2, 3Py5COO, F3COO, 3Py 6NHAc. F4C, F40Me, Nal2, F3aao, F4aa, F4aao, 3Py6NHaa, 5Pdo, F3CON. F4F, F4OEt, F4Me, F4CON, F4CONPEG4Me, F30Me, YaeCOpipzaa, F4aaopipzaa, 4Pdo, 3Py6CON, Atp, Cha4cH, Cha4tH. Cha4cOMe, A1mor, F4amCOpipzaa. Chg, Tbg, Gepr, Gepe. Acpr, Cba, Gthp, NIeCOO, NleOH, Atb, Nva, Nle, DapAc, Abu, Ncit, dMeS, TdMe, Cbg, NvaOMe, NIeOMe, AhpOMe, F4u, F4CONdMe, F4 ms, F34dOMe, F3C, F3CONdMe, 5Inda, F3aa, 3Py6NHae, 3Py60Me, MeNal2, MeNal27N. MeF34diox, MeF34dOMe, MeF4T. MeW7N, MeF3C4Me. MeF3Me4C, Bph, 3Py6Ph, F41Me4Pyz, F43Pyz, F44Pyz, F41Pyz, F41Me3Pyz, F41Et4Pyz, F41MeOe4Pyz, F41MeOp4Pyz, F44thp, F4Ac4Pip, PhNva, PhNle, Yph, Ybn. F4tb, F4OPr. S3REt, Yae, datb, Me3Py, W5N, MeA4paa. MeI, MeA, MeG, MeV, MeT, alI, TMe, MeQ, MeTMe, MeK, MeKAc, Har, KAc, dd, EtG, K(biotin), MeeG, CmpG, Nmm, Ndm, SMe, HseMe, SiPr, SPr, MeW1Me, MeQdMe, MeSMe, MeHseMe, MeKCOpipzaa, KCOpipzetOH, KCOmeglunine, MeK(de), MeK(H), MeK(df), MeK(datb), and the like. Note that D-amino acids such as da may be classified as D-amino acids, but they may also be classified according to the properties of their side chains, and N-methyl amino acids may be classified as N-alkyl amino acids and may also be classified according to the property of the side chain.

In some embodiments, the unnatural amino acids incorporated into the peptides include one or more of: 1) a ketone functional group (as found in para or meta acetyl-phenylalanine) that can be specifically reacted with hydrazines, hydroxylamines and their derivatives (Addition of the keto functional group to the genetic code of Escherichia coli Wang L. Zhang Z, Brock A, Schultz P G. Proc Natl Acad Sci USA. 2003 Jan. 7; 100(l):56-61; Bioorg Med Chem Lett. 2006 Oct. 15; 16(20):5356-9. Genetic introduction of a dike tone-containing amino acid into proteins. Zeng H, Xie J, Schultz P G), 2) azides (as found in p-azido-phenylalanine) that can be reacted with alkynes via copper catalyzed “click chemistry” or strain promoted (3+2) cycloadditions to form the corresponding triazoles (Addition of p-azido-L-phenylalanine to the genetic code of Escherichia coli. Chin J W, Santoro S W, Martin A B, King D S, Wang L, Schultz P G. J Am Chem Soc. 2002 Aug. 7; 124(31):9026-7; Adding amino acids with novel reactivity to the genetic code of Saccharomyces cerevisiae. Deiters A, Cropp T A, Mukherji M, Chin J W, Anderson J C, Schultz P G. J Am Chem Soc. 2003 Oct. 1:125(39):11782-3), or azides that can be reacted with aryl phosphines, via a Staudinger ligation (Selective Staudinger modification of proteins containing p-azidophenylalanine. Tsao M L, Tian F, Schultz P G. Chembiochem, 2005 December; 6(12):2147-9), to form the corresponding amides, 3) alkynes that can be reacted with azides to form the corresponding triazole (In vivo incorporation of an alkyne into proteins in Escherichia coli. Deiters A, Schultz P G. Bioorg Med Chem Lett. 2005 Mar. 1; 15(5):1521-4), and 4) boronic acids (boronates) than can be specifically reacted with compounds containing more than one appropriately spaced hydroxyl group or undergo palladium mediated coupling with halogenated compounds (Angew Chem Int Ed Engl. 2008: 47(43):8220-3. A genetically encoded boronate-containing amino acid . . . . Brustad E, Bushey M L, Lee J W, Groff D, Liu W. Schultz P G).

The peptide of the present disclosure embraces various derivatives thereof. Examples of the derivatives include derivatives having an amide, ester, or carboxyl group as the C-terminus and/or N-terminus thereof. Additional examples of the derivatives of the peptide include those obtained by modification such as phosphorylation, methylation, acetylation, adenylylation, ADP-ribosylation, or glycosylation and fused protein obtained by fusion with another peptide or protein. These derivatives can be prepared by those skilled in the art in a known manner or a method based thereon.

In some embodiments, the peptide described herein comprises a basic amino acid. Examples of the basic amino acid include arginine, lysine, citrulline, ornithine, creatine, histidine, diaminobutanoic acid, and diaminopropionic acid.

In some embodiments, provided herein is a peptide having 90% or more sequence identity to any of sequences disclosed herein. In some embodiments, the sequence identity is at least 95% or 99%.

In some embodiments, the peptide is bicyclic or polycyclic. In some embodiments, a conjugate described herein comprises a bicyclic peptide. Exemplary bicyclic peptides include the bicyclic targeting peptides of BT5528, BT1718, and BT8009. Exemplary bicyclic peptides are described in US20180200378, U.S. Ser. No. 10/441,663, U.S. Pat. No. 8,680,022B2, US20180280525, and US20200215199, each of which is hereby incorporated by reference in its entirety. In some cases, when a peptide is cyclized, protease resistance is improved, metabolic stability is improved, and restrictions are also added to conformational change, so that rigidity is increased and membrane permeability and affinity for the target protein is improved.

In some embodiments, the peptide of the present disclosure has a cyclic structure in which a chloroacetylated amino acid and a cysteine or cysteine variant such as MeC residue present in the peptide are bound. In one aspect, the peptide has a cyclic structure in which an N-terminal amino acid and a cysteine or cysteine variant such as MeC residue present in the peptide are bound. In some embodiments, the peptide has a cyclic structure in which an N-terminal amino acid and the 12^{th} cysteine or cysteine variant such as MeC residue present in the peptide are bound. In some embodiments, the peptide has a cyclic structure in which a chloroacetylated N-terminal amino acid and the 12^{th} cysteine or cysteine variant such as MeC residue present in the peptide are bound. “Chloroacetylation” may be replaced with “haloacetylation” using another halogen. Furthermore, “acetylation” may be “acylation” using an acyl group other than an acetyl group.

In some embodiments, the peptide is a lasso peptide. Lasso peptides can be synthetic or naturally produced by bacteria, and they possess a distinctive threaded lariat fold that offers a 3D array of functionality for engaging biological targets. This lasso structure can enable beneficial properties such as affinity, stability and potent biological activities. Suitable lasso structure can be designed by algorithms. Exemplary lasso peptides are provided in Hegemann, J. D., et al., Lasso Peptides: An Intriguing Class of Bacterial Natural Products, Acc. Chem. Res., 2015, 48, 1909-1919; Tietz, J. L, et al., A new genome-mining tool redefines the lasso peptide biosynthetic landscape. Nature Chem Bio, 2017, 13, 470-478; DiCaprio, A. J., et al., Enzymatic Reconstitution and Biosynthetic Investigation of the Lasso Peptide Fusilassin. J. Am. Chem. Soc., 2019, 141, 290-297: A1 Tonma, R. S., et al., Site-Directed and Global Incorporation of Orthogonal and Isostructural Noncanonical Amino Acids into the Ribosomal Lasso Peptide Capistruin, ChemBioChem, 2015, 16, 503-509.

Further exemplary peptides include BMS-753493. Somatostatius, Octreotide, Octreotate, Lanreotide, Pasireotide, JR-11, L-779,976, BIM-23120, Satoreotide, depreotide, 18F-KYNDRLPLYISNP (SEQ ID NO: 399), CaIX-PI, and FAP-2286.

The radiopharmaceutical conjugates of the present disclosure embrace salts thereof. Physiologically acceptable base or acid are used to create salt forms of the radiopharmaceutical conjugates. Examples include addition salts with an inorganic acid (such as hydrochloric acid, hydrobromic acid, hydroiodic acid, sulfuric acid, or phosphoric acid), addition salts with an organic acid (such as p-toluenesulfonic acid, methanesulfonic acid, oxalic acid, p-bromophenylsulfonic acid, carboxylic acid, succinic acid, citric acid, benzoic acid, or acetic acid), inorganic bases (such as ammonium hydroxide, alkali or alkaline earth metal hydroxide, carbonate, or bicarbonate), and an amino acid.

In certain embodiments, the peptides and radiopharmaceutical conjugates described herein comprises one or more unnatural amino acids that are not any one of the 20 canonical amino acids found in proteins. Representative unnatural amino acids that can be incorporated into the peptides and conjugates described herein are provided in the table below.

TABLE 5G
Structures of exemplary unnatural amino acids
that can be incorporated into a peptide/conjugate described herein

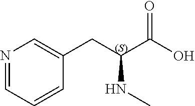

Me3Py

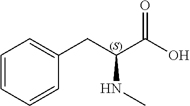

MeF

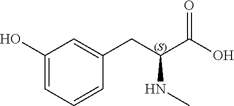

MeF3H

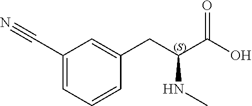

MeF3CN

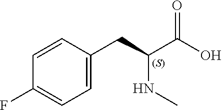

MeF4F

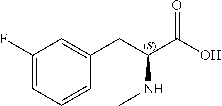

MeF3F

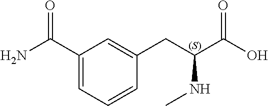

MeF3CON

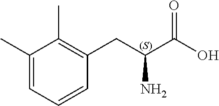

F23dMe

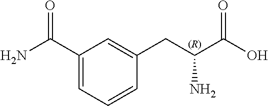

df3CON

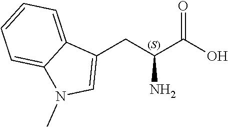

W1Me

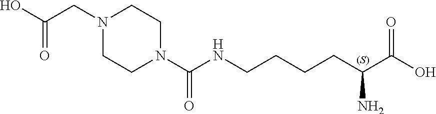

KCOpipzaa

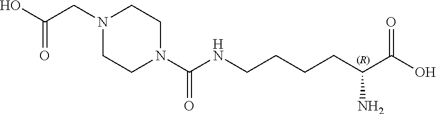

dkCOpipzaa

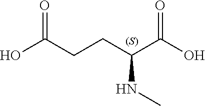

MeE

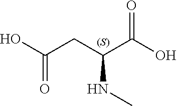

MeN

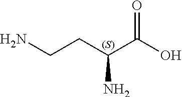

Dab

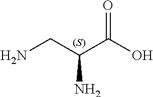

Dap

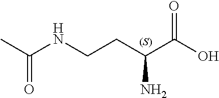

DabAc

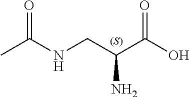

DapAc

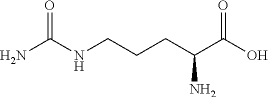 [SMILES: NC(=O)NCCC[C@H](N)C(=O)O]

Cit

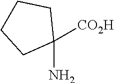

Cle

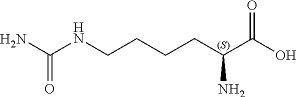

hCit

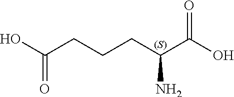

Hgl

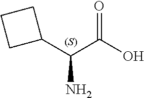

Cbg

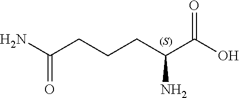

Hgn

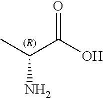

da

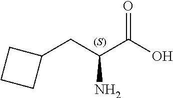

Cba

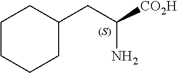

Cha

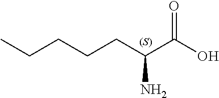

Ahp

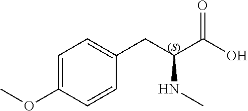

MeF4OMe

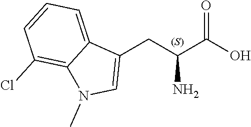

W1Me7Cl

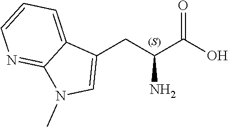

W1Me7N

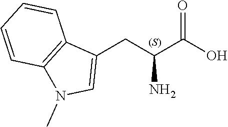

WIEt

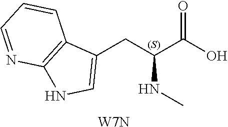

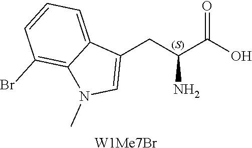

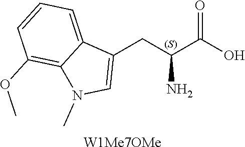

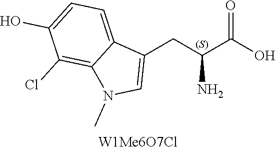

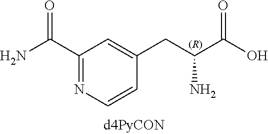

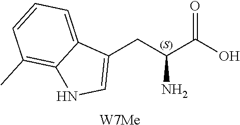

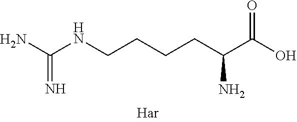 [SMILES: N=C(N)NCCCC[C@H](N)C(=O)O]

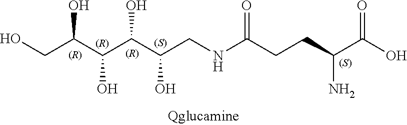

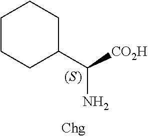

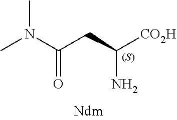

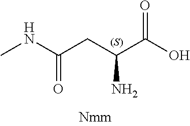

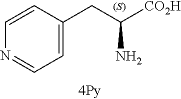

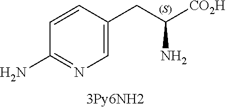

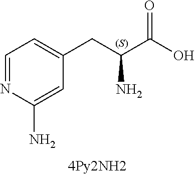

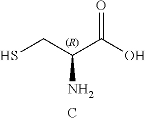

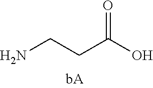

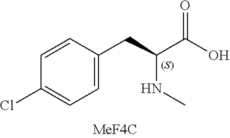

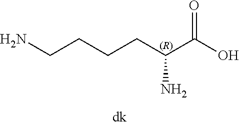

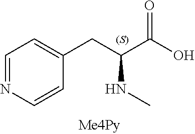

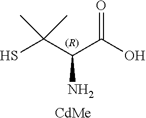

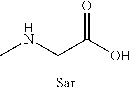

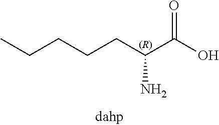

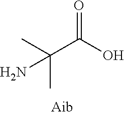

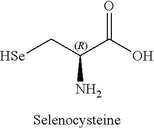

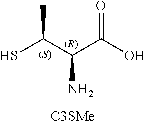

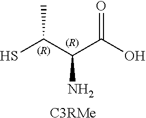

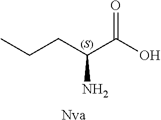

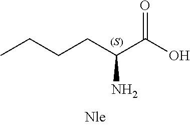

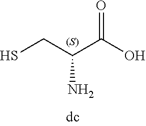

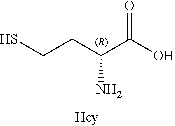

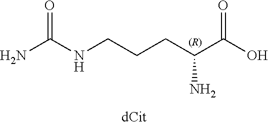

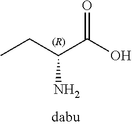

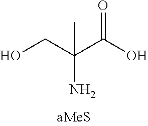

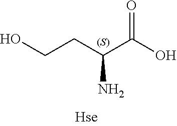

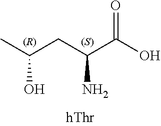

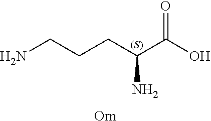

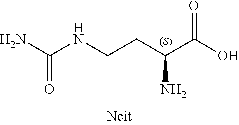

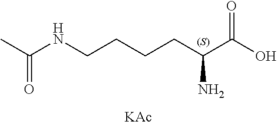

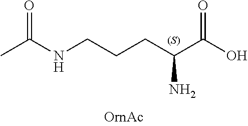

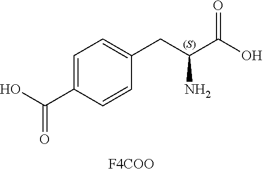

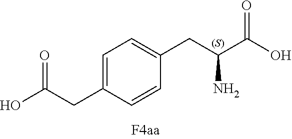

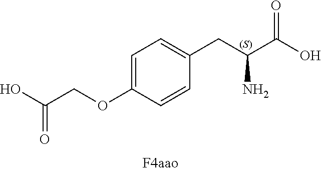

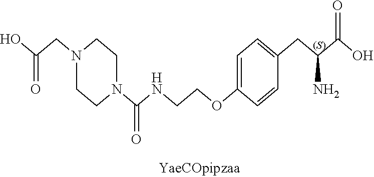

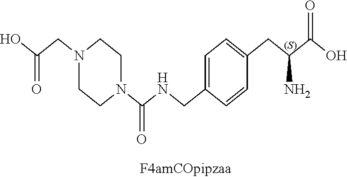

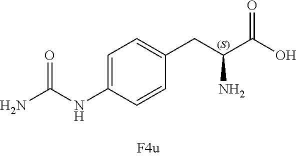

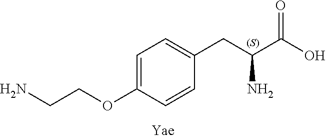

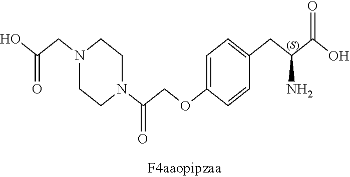

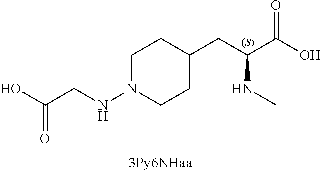

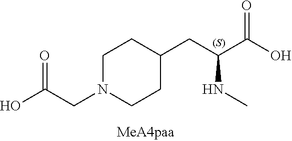

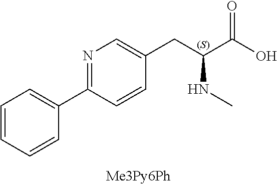

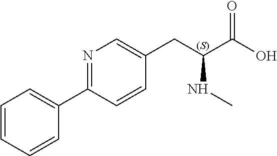

3Py6Ph

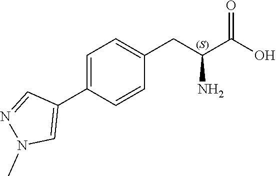

F41Me4Pyz

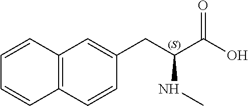

MeNal2

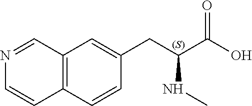

MeNal27N

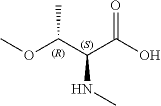

TMe

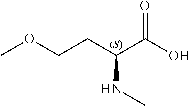

MeHseMe

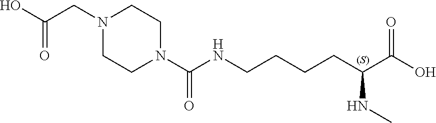

MeKCOpipzaa

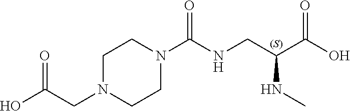

MeDapCOpipzaa

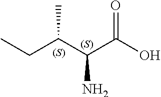

I

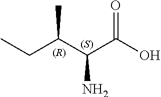

aII

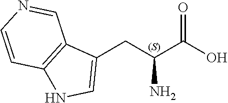

W5N

### Linker

A conjugate described herein can comprise one or more linkers. In some embodiments, the linker covalently attaches the peptide with the metal chelator. In some embodiments, the peptide attaches directly to the metal chelator without a linker. In some embodiments, the present disclosure describes linkers that function as a spacer.

A radiopharmaceutical conjugate described herein can comprise a linker. The linker can comprise a number of intervening atoms (on a linear chain, excluding pendant groups or substituents) between the metal chelator or covalently bound radionuclide and the binding peptide described herein, thereby creating a distance between the metal chelator or covalently bound radionuclide and the binding peptide. In some embodiments, a linker comprises 10-100 intervening atoms between the metal chelator or covalently bound radionuclide and the binding peptide. In some embodiments, a linker comprises 2-60 intervening atoms between the metal chelator or covalently bound radionuclide and the binding peptide. In some embodiments, a linker comprises 2 to 20, 2 to 50, 5 to 15, 5 to 25, 10 to 40, 30 to 60, or 10 to 20 intervening atoms between the metal chelator or covalently bound radionuclide and the binding peptide. In some embodiments, a linker comprises 3 to 30 intervening atoms between the metal chelator or covalently bound radionuclide and the binding peptide. In some embodiments, a linker comprises 5 to 25 intervening atoms between the metal chelator or covalently bound radionuclide and the binding peptide. In some embodiments, a linker comprises 6 to 18 intervening atoms between the metal chelator or covalently bound radionuclide and the binding peptide. In some embodiments, a linker comprises 10 to 20 intervening atoms between the metal chelator or covalently bound radionuclide and the binding peptide. The intervening atoms can comprise 1 or more carbons, and optionally one or more heteroatoms such as O and N. In some embodiments, the intervening atoms comprise 2 to 20, 2 to 50, 5 to 15, 5 to 25, 10 to 40, 30 to 60, or 10 to 20 carbons. In some embodiments, the intervening atoms comprise 0, 1, 2, 3, 4, 5, or 6 nitrogen. In some embodiments, the intervening atoms comprise 0, 1, 2, 3, 4, 5, 6, 7 or 8 oxygen. In some embodiments, the intervening atoms comprise 1 to 6 nitrogen and 0 to 4 oxygen.

A linker can comprise one or more amino acid residues. In some embodiments, the linker comprises 1 to 3, 1 to 5, 1 to 10, 5 to 10, or 5 to 20 amino acid residues. In some embodiments, the linker comprises 1, 2, 3, 4, 5, 6, 7, 8, 9, or 10 amino acid residues. In some embodiments, the linker comprises 1 to 5 amino acid residues. For example, the linker can comprise one or more lysine (K) residues such as K, KK, or KKK sequences. In some embodiments, the linker comprises a lysine or a derivative thereof. In some embodiments, the linker comprises a lysine. In some embodiments, one or more amino acids of the linker are unnatural amino acids. In some embodiments, the linker comprises a lysine residue, an alanine residue, or both. In certain embodiments, the linker comprises one or more amino acids chosen from a lysine residue, an alanine residue, or a phenylalanine residue. In some embodiments, the linker comprises a lysine residue. In some embodiments, the linker comprises an alanine residue. In some embodiments, the linker comprises a lysine residue, an alanine residue, a glycine residue, a D-phenylalanine residue, a histidine residue, a dAtb residue, or a D-glutamate residue. In some embodiments, the linker comprises 2 amino acids selected from the group consisting of lysine, alanine, glycine, D-phenylalanine, histidine, dAtb, and D-glutamate.

A herein-described linker can attach to the N-terminus of the peptide, the C-terminus of the peptide, or a non-terminal amino acid of the peptide, or it can attach to the peptide through a combination of the above. In some embodiments, the linker is attached to the peptide via its N-terminus. In some embodiments, the linker is attached to the peptide via a cysteine residue at the C-terminus. In some embodiments, the linker is attached to the peptide via a cysteine residue at the N-terminus. In some embodiments, the linker is attached to the peptide via its C-terminus. In some embodiments, the linker is attached to the peptide via a non-terminal amino acid. The linker can be bonded to the peptide, the payload molecule, or both, for example, through a chemically reactive group. Exemplary chemically reactive groups include, but are not limited to, a free amino, imino, hydroxyl, thiol or carboxyl group (e.g., to the N- or C-terminus, to the epsilon amino group of one or more lysine residues, the free carboxylic acid group of one or more glutamic acid or aspartic acid residues, or to the sulfhydryl group of one or more cy steinyl residues). The site to which the linker is bound to the peptide can be a natural or unnatural amino acid of the peptide and/or it can be introduced into the peptide, e.g., by DNA recombinant technology (e.g., by introducing a cysteine or protease cleavage site in the amino acid sequence) or by protein biochemistry (e.g., reduction, pH adjustment or proteolysis). Exemplary methods for attaching the linker includes carbodiimide reaction, reactions using bifunctional agents such as dialdehydes or imidoesters, Schiff base reaction. Suzuki-Miyaura cross-coupling reactions. Isothiocyanates as coupling agents, and click chemistry.

The linker can have a prescribed length thereby linking the payload molecule and the peptide while allowing an appropriate distance therebetween. In some embodiments, the linker has 1 to 100 atoms, 1 to 60 atoms, 1 to 30 atoms, 1 to 15 atoms, 1 to 10 atoms, 1 to 5, or 2 to 20 atoms in length. In some embodiments, the linker has 1 to 10 atoms in length.

The linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences (e.g., EAAAK (SEQ ID NO: 400)), proline-rich sequences, and regions rich in double and/or triple bonds.

In some embodiments, a linker may be further added to the cyclic peptide. Examples of the linker include an amino acid linker (peptide linker), a chemical linker, a fatty acid linker, a nucleic acid linker, a sugar chain linker, or the like, or it may be a complex, for example, a chemical linker, a peptide linker, or the like. Examples of the chemical linker include a PEG (polyethylene glycol) linker. For example, the PEG linker may comprise between 1 to 24 ethylene glycol units. Furthermore, the linker may be a fatty acid linker containing a divalent chemical moiety derived from a fatty acid. The linker includes at least one amino acid, and, for example, a glycine-rich peptide such as a peptide having a sequence [Gly-Gly-Gly-Gly-Ser]n (in the formula, n is 1, 2, 3, 4, 5, or 6) (SEQ ID NO: 401) such as that according to U.S. Pat. No. 7,271,149, incorporated by reference herein, or a serine-rich peptide linker according to U.S. Pat. No. 5,525,491, incorporated by reference herein, may be used. In a non-limiting manner, there are some cases where a physical property (for example, solubility) of the peptide may be changed by the addition of a linker. In one aspect, the amino acid linker includes an amino acid sequence according to any one of SEQ ID NOs: 1 to 72.

The linker may be added at any position. In some embodiments, the linker may be bound to Cys positioned on the C-terminal side or may be bound to an amino acid comprised in the cyclic peptide. In some embodiments, the linker is bound to Cys or variant thereof positioned on the C-terminal side. In some embodiments, the linker is added to the —COOH on the Cys or variant residue. It is possible to add one to several amino acids to the C-terminus of such Cys or cysteine variant, such as MeC, and then the liker is added to its terminus: for example, Gly is added to the C-terminus of Cys or cysteine variant, such as MeC, within the cyclic structure peptide, then the —COOH of the Gly is bound to linker, such as a PEG linker or an amino acid linker. In some embodiments, the linker is added to the side chain of an amino acid within the cyclic peptide. In some embodiments, the linker is added to the side chain of a lysine within the cyclic peptide. In some embodiments, the linker is added to the side chain of X1, X4, X5, X8, or X12 of Formula (I).

The linker can be cleavable, e.g., under physiological conditions, e.g., under intracellular conditions, such that cleavage of the linker releases the payload molecule in the intracellular environment. The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin. In other embodiments, the linker is not cleavable. In some embodiments, the linker is pH-sensitive, i.e., sensitive to hydrolysis at certain pH values. For example, the pH-sensitive linker can be hydrolyzable under acidic conditions. For example, a linker can be an acid-labile linker that is hydrolyzable in the lysosome (e.g., a hydrazone, semicarbazone, thiosemicarbazone, cis-aconitic amide, orthoester, acetal, ketal, or the like). Such linkers can be relatively stable under neutral pH conditions, such as those in the blood, but are unstable at below pH 5.5 or 5.0, the approximate pH of the lysosome. In some embodiments, the hydrolyzable linker is a thioether linker.

In some embodiments, the linker comprises an amino acid sequence, such as a combination of amino acid sequence and a flexible and/or rigid region.

In some embodiments, the linker comprises one or more of substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. In some embodiments, the linker comprises substituted or unsubstituted C₁-C₃₀ alkylene. In some embodiments, the linker comprises substituted or unsubstituted C₁-C₃₀ heteroalkylene. In some embodiments, the linker comprises polyethylene glycol such as (—CH₂—CH₂—O—)₁₋₁₀.

In some embodiments, the linker has a structure of

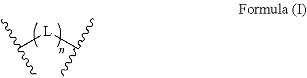

wherein each L is independently —O—, —NR^{L}—, —N(R^{L})₂⁺—, —OP(═O)(OR^{L})O—, —S—, —S(═O)—, —S(═O), ═CH—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, substituted or unsubstituted C₁-C₃₀heteroalkylene, —(C₁-C₃₀ alkylene)-O—, —O—(C₁-C₃₀ alkylene)-, —(C₁-C₃₀ alkylene)-NR^{L}, —NR^{L}—(C₁-C₃₀ alkylene)-, —(C₁-C₃₀ alkylene)-N(R^{L})₂⁺—, —N(R^{L})₂⁻, —(C₁-C₃₀ alkylene)-, or a click chemistry residue; and
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₈ cycloalkyl, substituted or unsubstituted C₂-C₇ heterocycloalkyl, substituted or unsubstituted aryl, or substituted or unsubstituted heteroaryl; and
n is 1 to 20 (e.g., 1, 2, 3, 4, 5, 6, 7, 8, 9, 10, 11, 12, 13, 14, 15, 16, 17, 18, 19, or 20).

In some embodiments, the linker has a structure of

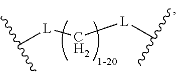

wherein each L is independently —O—, —NR^{L}—, —N(R^{L})₂⁺—, —OP(═O)(OR^{L})O—, —S—, —S(O)—, —S(═O)₂—, —CH—CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}S(═O)₂—, —S(═O)₂NR^{L}—, —C(═O)NR^{L}S(═O)₂—, or S(═O)₂NR^{L}C(═O)—.

In some embodiments, the linker of Formula (II-1) has a structure of Formula (II-1a),

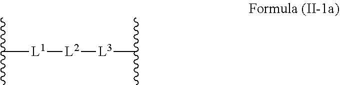

wherein
   each of L¹ and L³ is independently —O—, —NR^{L}—, —N(R^{L})₂—, —OP(═O)(R^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}, —NR^{L}C(═O)—, —OC(═O)NR^{L}—, —NR^{L}C(═O)O—, —NR^{L}C(═O)NR^{L}—, —NR^{L}S(═O)₂, —S(═O)₂NR^{L}—, —C(═O)NR^{L}S(═O)₂—, or —S(═O)₂NR^{L}C(═O)—; and
   L² is absent, substituted or unsubstituted C₁-C₃₀ alkylene, or substituted or unsubstituted C₁-C₃₀ heteroalkylene.

In some embodiments, the linker comprises a structure of Formula (II-1b),

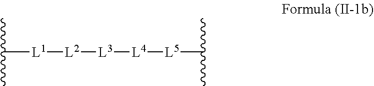

wherein
   each of L¹ and L⁵ is independently —O—, —NR^{L}—, —N(R^{L})₂—, —OP(═O)(OR^{L})O—, —S—, —S(═O)—, —S(═O)₂—, —CH═CH—, ═CH—, —C≡C—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(O)O—, —NR^{a}C(═O)NR^{L}—, —NR^{L}S(═O)₂—, —S(═O₂NR^{L}—, —C(═O)NR^{L}S(═O)₂—, —S(═O)₂NR^{L}C(═O)—, substituted or unsubstituted 5-6 membered cycloalkyl, or substituted or unsubstituted 5-6 membered heterocycloalkyl; and
   L², L³ and L⁴ are each independently absent, substituted or unsubstituted 5-6 membered cycloalkyl, substituted or unsubstituted 5-6 membered heterocycloalkyl, substituted or unsubstituted C₁-C₃₀ alkylene, or substituted or unsubstituted C₁-C₃₀ heteroalkylene.

In some embodiments, L¹ is —NH—.

In some embodiments, L² is absent. In some embodiments, L² is substituted or unsubstituted C₁-C₃₀ alkylene, or substituted or unsubstituted C₁-C₃₀ heteroalkylene. In some embodiments, L² is substituted or unsubstituted C₁-C₃₀alkylene. In some embodiments, L² is substituted or unsubstituted C₁-C₃₀ heteroalkylene. In some embodiments. L² is substituted or unsubstituted C₁-C₃₀ alkylene, or substituted or unsubstituted C₁-C₃₀ heteroalkylene. In some embodiments, L² is optionally substituted. In some embodiments, L² is optionally substituted with one or more substituents selected from —OH, —SH, oxo, amino, C₁-C₆ alkyl, C₁-C₆ hydroxyalkyl, C₁-C₆ haloalkyl, C₁-C₆ aminoalkyl, —C(═O)OR^{L}, —OC(═O)R^{L}, —OC(═O)OR^{L}, —C(═O)N(R^{L})₂, —NR^{L}C(═O)R^{L}, —OC(═O)N(R^{L})₂, and —NR^{L}C(═O)OR^{L}. In some embodiments, L² is C₁-C₃₀ heteroalkylene that is optionally substituted with one or more substituents selected from —OH, —SH, oxo, amino, C₁-C₆alkyl, C₁-C₆hydroxyalkyl, C₁-C₆haloalkyl, and C₁-C₆aminoalkyl. In some embodiments, L2 is optionally substituted with C₁-C₆ alkyl which is further optionally substituted with one or more substituents chosen from —OH, —SH, oxo, amino, C₆-C₁₀ aryl, 6-to 10-membered heteroaryl, —C(═O)OR^{L}, —OC(═O)R^{L}, —OC(═O)OR^{L}, —C(═O)N(R^{L})₂, —NR^{a}C(═O)R^{L}, —OC(═O)N(R^{L})₂, and —NR^{L}C(═O)OR^{L}.

In some embodiments, L³ is —NH—. In some embodiments, L³ is absent.

In some embodiments, L⁴ is absent. In some embodiments, L⁴ is substituted or unsubstituted 5-6 membered cycloalkyl, substituted or unsubstituted 5-6 membered heterocycloalkyl, substituted or unsubstituted C₁-C₃₀ alkylene, or substituted or unsubstituted C₁-C₃₀ heteroalkylene.

In some embodiments, L⁵ is —NH—. In some embodiments, L⁵ is absent.

In some embodiments for Formula (II-1b), L⁵ is —O—, —N(methyl)-, —NH— or —C(═O)—; L⁵ is —O—. —N(methyl)-, —NH— or —C(═O)—; L², L³ and L⁴ are each independently absent, substituted or unsubstituted 5-6 membered cycloalkyl, substituted or unsubstituted 5-6 membered heterocycloalkyl, substituted or unsubstituted C₁-C₁₂ alkylene, or substituted or unsubstituted C₁-C₃₀ heteroalkylene, wherein L¹ is connected to the payload molecule and L⁵ is connected to the GPC3 binding peptide.

In some embodiments for Formula (II-1b), L² is unsubstituted C₁-C₁₂ alkylene, and L³ and L⁴ are absent.

In some embodiments, the linker comprises substituted or unsubstituted C₁-C₃₀ alkylene, C₁-C₁₂ alkylene, C₁-C₈ alkylene, C₁-C₆ alkylene, or C₂-C₆ alkylene. In some embodiments, the linker comprises C₂-C₆ alkylene. In some embodiments, the linker comprises C₄-C₆ alkylene.

In some embodiments, each of L¹ is independently —O—, —NR^{L}—, —N(R^{L})₂—, —OP(═O)(OR^{a})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{a}C(═O)NR^{L}—, —NR^{a}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₅ cycloalkyl, substituted or unsubstituted C₁-C₁₂ heterocycloalkyl, substituted or unsubstituted aryl, substituted or unsubstituted heteroaryl substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene. In some embodiments, L¹ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L} C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂, or —S(═O)₂NR^{L}C(═O)—. In some embodiments, L¹ is —O—, —NH—, —S(═O)—, —S(═O)—, or —C(═O)—. In some embodiments, L¹ is —C(═O)NH— or —NHC(═O)—. In some embodiments, L¹ is substituted or unsubstituted C₃-C₁₅ cycloalkyl, or substituted or unsubstituted C₁-C₂ heterocycloalkyl. In some embodiments, L¹ is substituted or unsubstituted aryl or substituted or unsubstituted heteroaryl. In some embodiments, L¹ is substituted or unsubstituted C₁-C₃₀ alkylene. In some embodiments, L¹ is substituted or unsubstituted C₂-C₃₀ alkenylene. In some embodiments. L¹ is substituted or unsubstituted C₁-C₃₀ heteroalkylene. In some embodiments. L¹ is substituted or unsubstituted C₅-C₂₅ heteroalkylene. In some embodiments, L¹ is substituted or unsubstituted C₅-C₁₂ heteroalkylene.

In some embodiments, each of L^{X2} is independently —O—, —NR^{L}—, —N(R)₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—. —OC(═O)NR^{L}—, —NR^{L}C(═O)O—, —NR^{a}C(═O)NR^{L}—, —NR^{L}C(═S)NR^{L}—, —CR^{L}═N—, —N═CR, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L² is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L^{X2} is —O—, —NH—, —S(═O)—, —S(═O)₂—, or —C(═O)—. In some embodiments, L² is —C(═O)NH— or —NHC(═O)—, In some embodiments, L² is substituted or unsubstituted C₃-C₁₅ cycloalkyl, or substituted or unsubstituted C₁-C₁₂ heterocycloalkyl. In some embodiments, L² is substituted or unsubstituted aryl or substituted or unsubstituted heteroaryl. In some embodiments, L² is substituted or unsubstituted C₁-C₃₀ alkylene. In some embodiments, L² is substituted or unsubstituted C₂-C₃₀ alkenylene. In some embodiments, L² is substituted or unsubstituted C₁-C₃₀ heteroalkylene. In some embodiments, L² is substituted or unsubstituted C₅-C₂₅ heteroalkylene. In some embodiments, L² is substituted or unsubstituted C₅-C₁₂ heteroalkylene.

In some embodiments, each of L³ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{a}C(═O)NR^{L}—, —NR^{L}C(═S)NR^{L}—, —CR^{L}═N—, —N═CR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L³ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L³ is —O—, —NH—, —S(═O)—, —S(═O)₂—, or —C(═O)—. In some embodiments, L³ is —C(═O)NH— or —NHC(═O)—. In some embodiments, L³ is substituted or unsubstituted C₃-C₁₅ cycloalkyl, or substituted or unsubstituted C₁-C₁₂ heterocycloalkyl. In some embodiments, L³ is substituted or unsubstituted aryl or substituted or unsubstituted heteroaryl. In some embodiments. L³ is substituted or unsubstituted C₁-C₃₀ alkylene. In some embodiments, L³ is substituted or unsubstituted C₂-C₃₀ alkenylene. In some embodiments. L³ is substituted or unsubstituted C₁-C₃₀ heteroalkylene. In some embodiments. L³ is substituted or unsubstituted C₅-C₂₅ heteroalkylene. In some embodiments, L³ is substituted or unsubstituted C₅-C₁₂ heteroalkylene. In some embodiments, L³ is absent.

In some embodiments, each of L⁴ is independently —O—, —NR^{L}—, —N(R^{a})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—. —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L^{X4} is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁴ is —O—, —NH—, —S(═O)—, —S(═O)₂—, or —C(═O)—. In some embodiments, L^{X4} is —C(═O)NH— or —NHC(═O)—, In some embodiments, L⁴ is substituted or unsubstituted C₃-C₁₅ cycloalkyl, or substituted or unsubstituted C₁-C₁₂ heterocycloalkyl. In some embodiments, L⁴ is substituted or unsubstituted aryl or substituted or unsubstituted heteroaryl. In some embodiments, L⁴ is substituted or unsubstituted C₁-C₃₀ alkylene. In some embodiments, L⁴ is substituted or unsubstituted C₂-C₃₀ alkenylene. In some embodiments, L⁴ is substituted or unsubstituted C₁-C₃₀ heteroalkylene. In some embodiments, L⁴ is substituted or unsubstituted C₅-C₂₅ heteroalkylene. In some embodiments, L⁴ is substituted or unsubstituted C₅-C₁₂ heteroalkylene. In some embodiments, L⁴ is absent.

In some embodiments, each of L⁵ is independently —O—, —NR^{L}—, —N(R^{L})₂—, —OP(═O)(OR^{L})O—, —S—, —S(═O)—, —S(═O)₂—, ═CH—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{a}C(═O)NR^{L}—, —NR^{L}C(═S)NR^{L}—, —CR^{L}═N—, —N═CR^{L}—, —NR^{L}S(═O)₂—, —S(═O)₂NR^{L}—, —C(═O)NR^{L}S(═O)₂—, —S(═O)₂NR^{L}C(═O)—, substituted or unsubstituted C₃-C₁₅ cycloalkyl, substituted or unsubstituted C₁-C₁₂ heterocycloalkyl, substituted or unsubstituted aryl, substituted or unsubstituted heteroaryl, substituted or unsubstituted C₁-C₃₀ alkylene, substituted or unsubstituted C₂-C₃₀ alkenylene, substituted or unsubstituted C₂-C₃₀ alkynylene, or substituted or unsubstituted C₁-C₃₀ heteroalkylene, In some embodiments, L⁵ is —O—, —NR^{L}—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —NR^{L}S(═O)₂—, —S(═O)₂NR^{L}—, —C(═O)NR^{L}S(═O)₂—, or —S(═O)₂NR^{L}C(═O)—. In some embodiments, L⁵ is —O—, —NH—, —S(═O)—, —S(═O)—, or —C(═O)—. In some embodiments, L⁵ is —C(═O)NH— or —NHC(═O)—. In some embodiments, L⁵ is substituted or unsubstituted C₃-C₁₅ cycloalkyl, or substituted or unsubstituted C₁-C₁₂ heterocycloalkyl. In some embodiments, L⁵ is substituted or unsubstituted aryl or substituted or unsubstituted heteroaryl. In some embodiments, L⁵ is substituted or unsubstituted C₁-C₃₀ alkylene. In some embodiments, L⁵ is substituted or unsubstituted C₂-C₃₀ alkenylene. In some embodiments. L⁵ is substituted or unsubstituted C₁-C₃₀ heteroalkylene. In some embodiments. L⁵ is substituted or unsubstituted C₅-C₂₅ heteroalkylene. In some embodiments, L⁵ is substituted or unsubstituted C₅-C₁₂ heteroalkylene. In some embodiments, L⁵ is absent.

In some embodiments, at least one L¹ is unsubstituted C₃-C₂₀ alkylene.

In some embodiments, the linker comprises one or more of a substituted or unsubstituted C₆-C₁₀aryl, substituted or unsubstituted C₅-C₉ heteroaryl, a sterol, sulfonamide, phosphate ester, polyethylene glycol, or C₃-C₂₀ alkylene, or amino acid residues.

In some embodiments, the linker comprises one or more selected from AEEA, AEEP, AEEEP, and AEEEEP groups. In some embodiments, the linker comprises

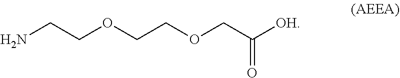 [SMILES: NCCOCCOCC(=O)O]

In some embodiments, the linker comprises

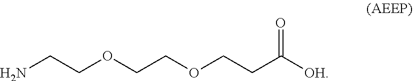 [SMILES: NCCOCCOCCC(=O)O]

In some embodiments, the linker comprises

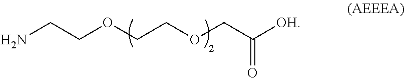

In some embodiments, the linker comprises

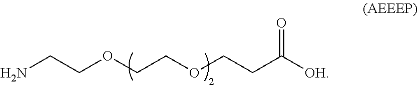

In some embodiments, the linker comprises a

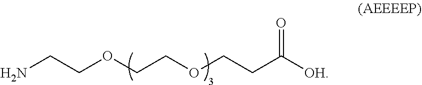

In some embodiments, the linker is

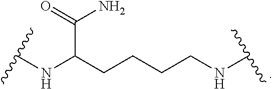 [SMILES: *NCCCCC(N*)C(N)=O]

In some embodiments, the linker is or comprises lysine. In some embodiments, the linker comprises C₁-C₁₂ alkylene. In some embodiments, the linker comprises C₃-C₉ alkylene. In some embodiments, the linker comprises C₂-C₈ alkylene. In some embodiments, the linker comprises 1 to 10 repeating ethylene glycol units. In some embodiments, the linker comprises 2 to 4 repeating ethylene glycol units. In some embodiments, the linker comprises 5 to 8 repeating ethylene glycol units. In some embodiments, the linker comprises NH₂—(CH₂)ₙ—COOH, wherein n is 1 to 12. In some embodiments, the linker comprises NH₂—(CH₂)₂—COOH. In some embodiments, the linker comprises NH₂—(CH₂)₆—COOH. In some embodiments, the linker comprises NH₂—(CH₂)₄—COOH. In some embodiments, the linker comprises NH₂—(CH₂)₅—COOH. In some embodiments, the linker comprises NH₂(CH₂)₆—COOH. In some embodiments, the linker comprises NH₂—(CH₂)₇—COOH. In some embodiments, the linker comprises NH₂—(CH₂)₈—COOH. In some embodiments, the linker comprises NH₂—(CH₂)₁₀—COH. In some embodiments, the linker is absent.

In some embodiments, a linker of the present disclosure (e.g., a linker of Formula (II-1), (II-1a) or (II-1b)) comprises a structure of

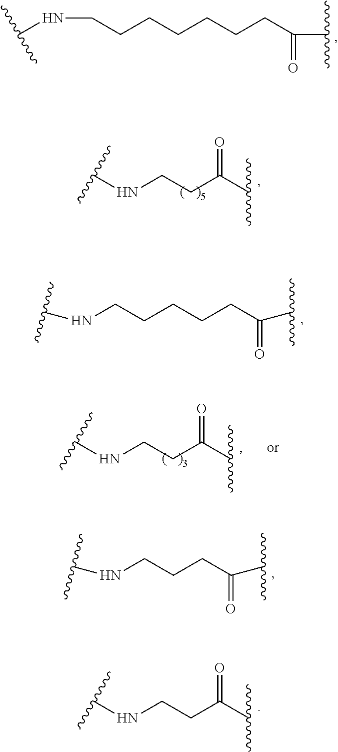

In some embodiments, a linker of the present disclosure comprises a structure of

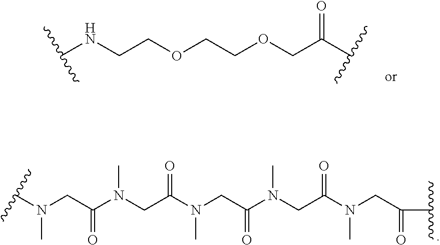 [SMILES: *NCCOCCOCC(*)=O ; *C(=O)CN(C)C(=O)CN(C)C(=O)CN(C)C(=O)CN(C)C(=O)CN(*)C]

In some embodiments, a linker of the present disclosure (e.g., a linker of Formula (II-1), (II-1a) or (II-1b)) comprises a structure of

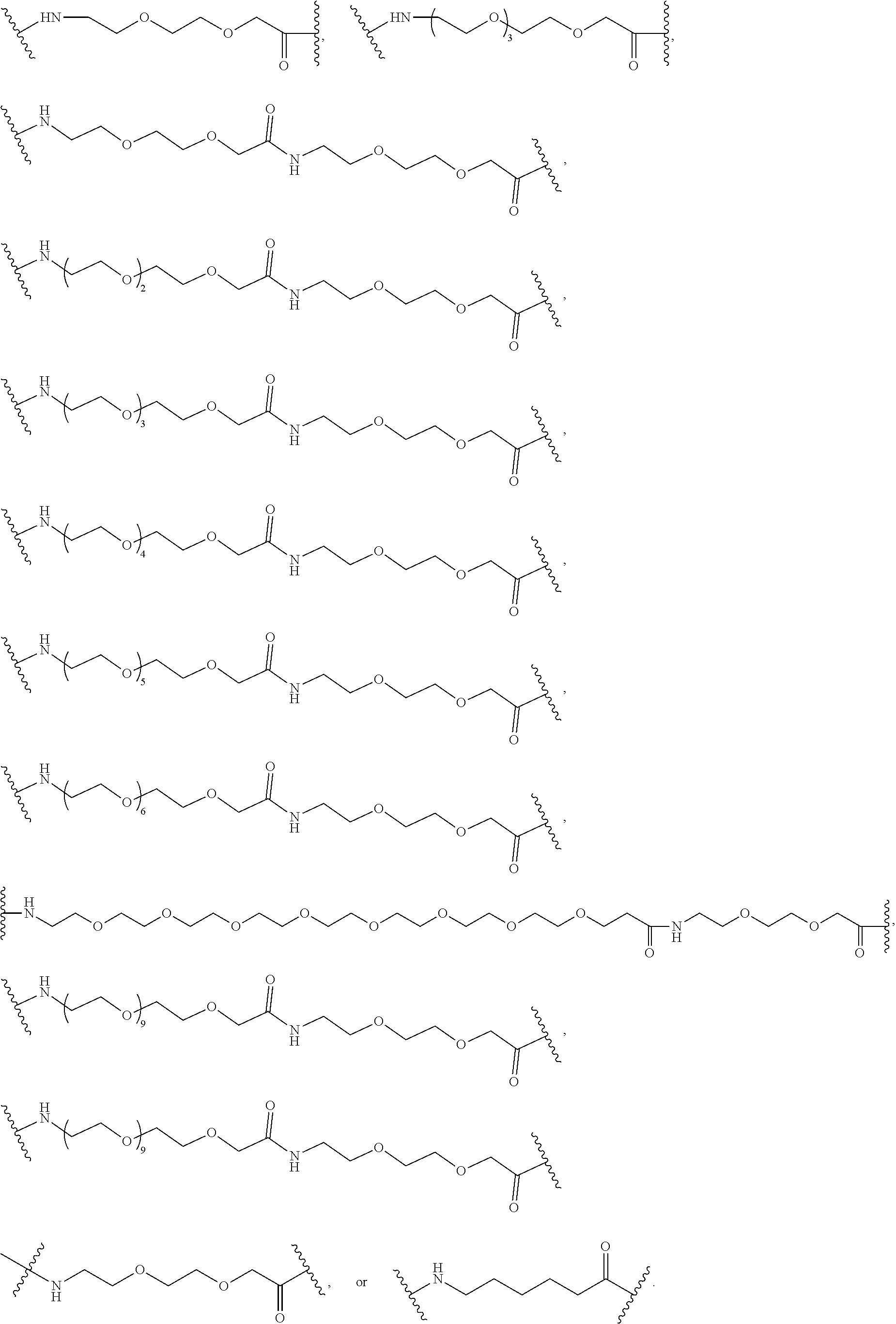

In some embodiments, the linker comprises a structure selected from:

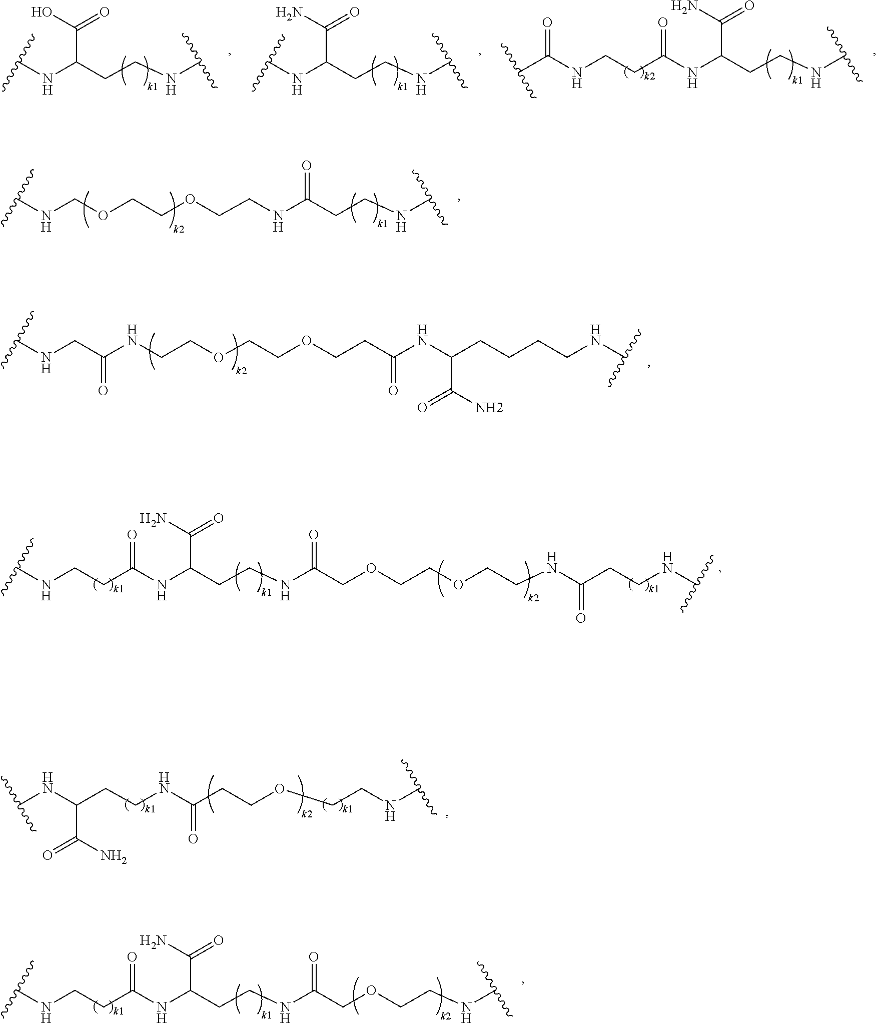

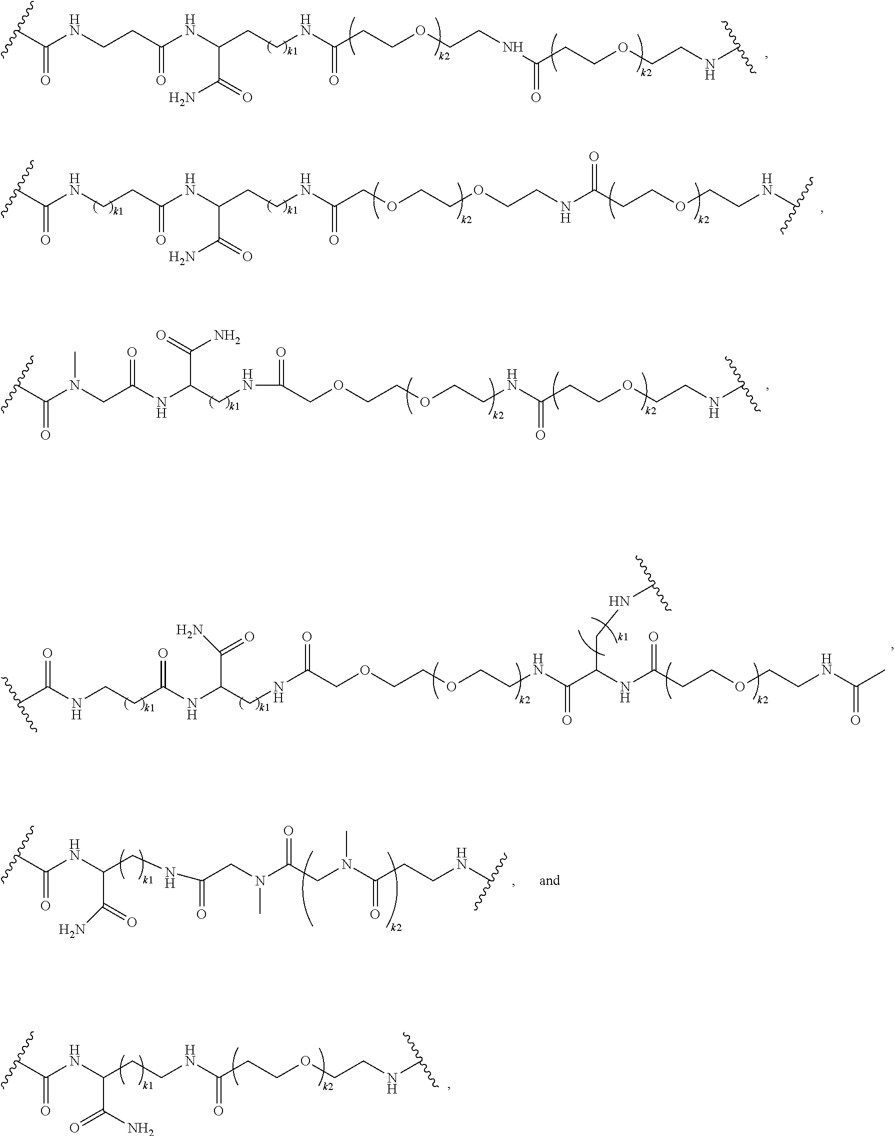

wherein each k1 is independently 0 or an integer from 1 to 20; and each k2 is independently 0 or an integer from 1 to 15. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments, k1 is 11. In some embodiments, k1 is 12. In some embodiments, k1 is 13. In some embodiments, k1 is 14. In some embodiments, k1 is 15. In some embodiments, k1 is 16. In some embodiments, k2 is 17. In some embodiments, k1 is 18. In some embodiments, k2 is 19. In some embodiments, k1 is 20. In some embodiments, k2 is 0. In some embodiments, k2 is 7. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10. In some embodiments, k2 is 11, In some embodiments, k2 is 12. In some embodiments, k2 is 13, in some embodiments, k2 is 14. In some embodiments, k2 is 15.

In some embodiments, the linker comprises a structure selected from:

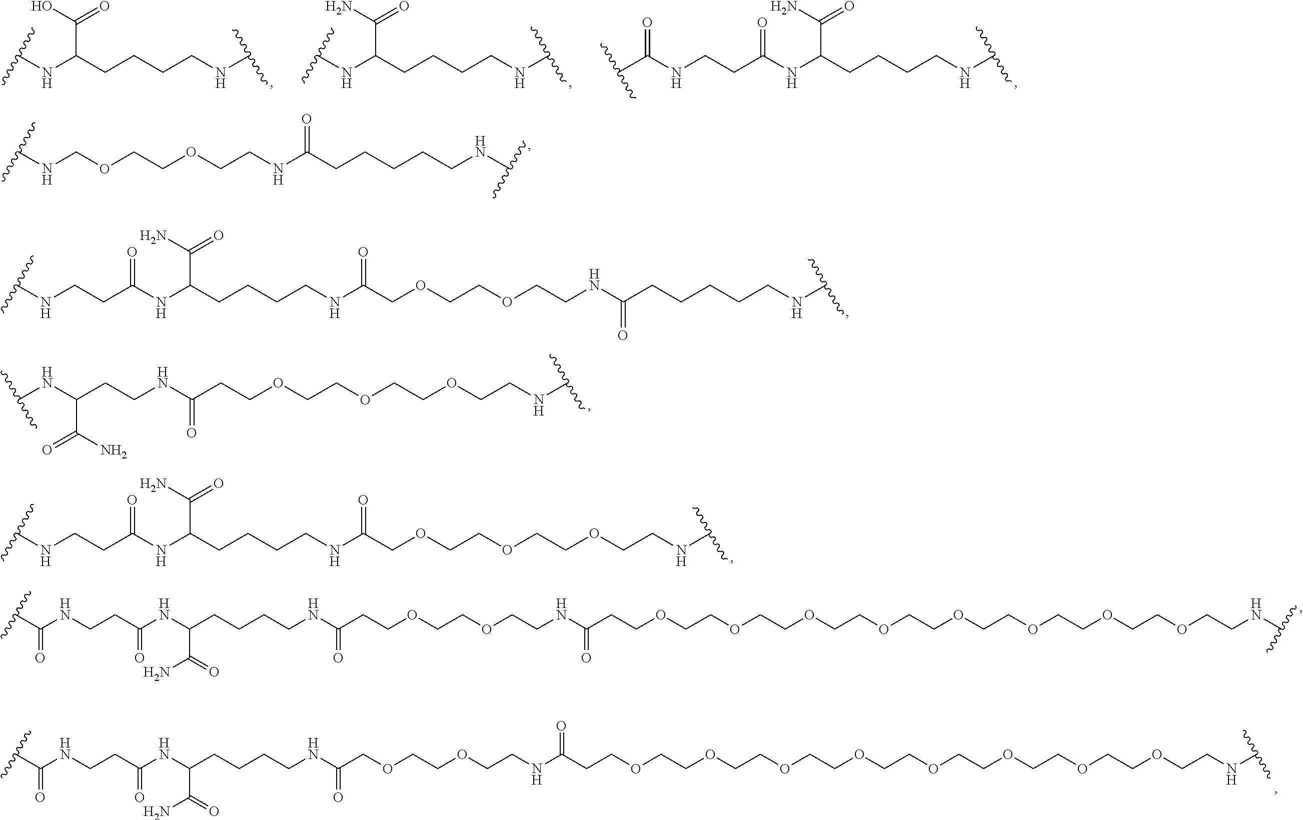

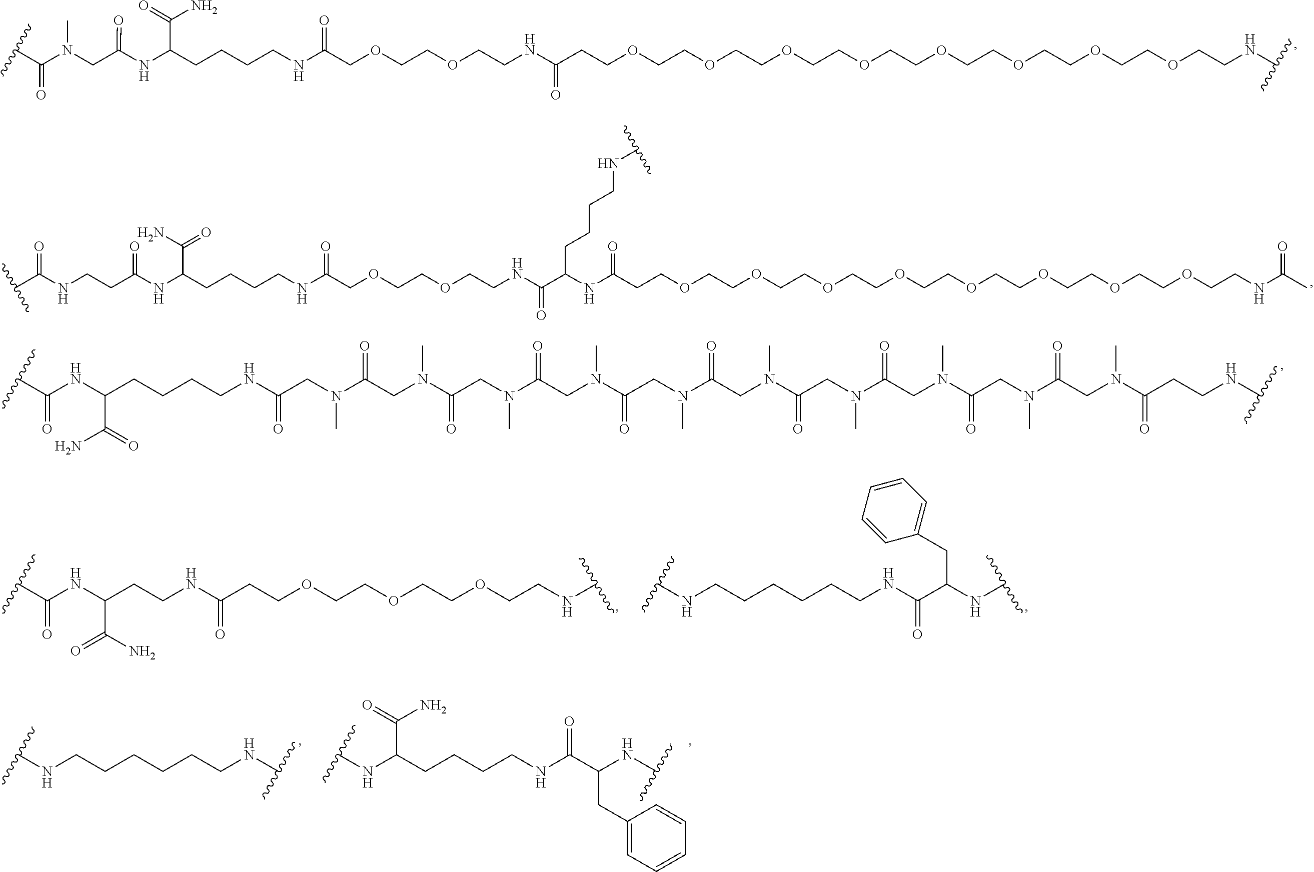

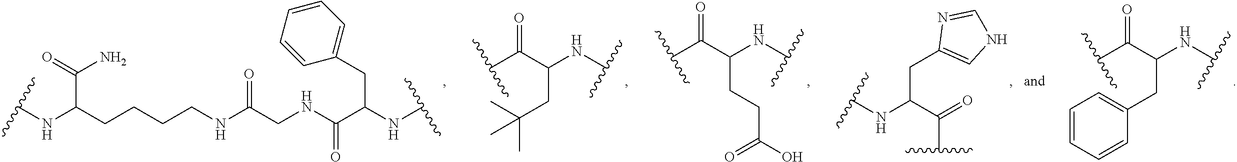 [SMILES: *NC(CCCCNC(=O)CNC(=O)C(Cc1ccccc1)N*)C(N)=O ; *NC(CC(C)(C)C)C(*)=O ; *NC(CCC(=O)O)C(*)=O ; *NC(Cc1c[nH]cn1)C(*)=O ; *NC(Cc1ccccc1)C(*)=O]

In some embodiments, the linker comprises a structure selected from:

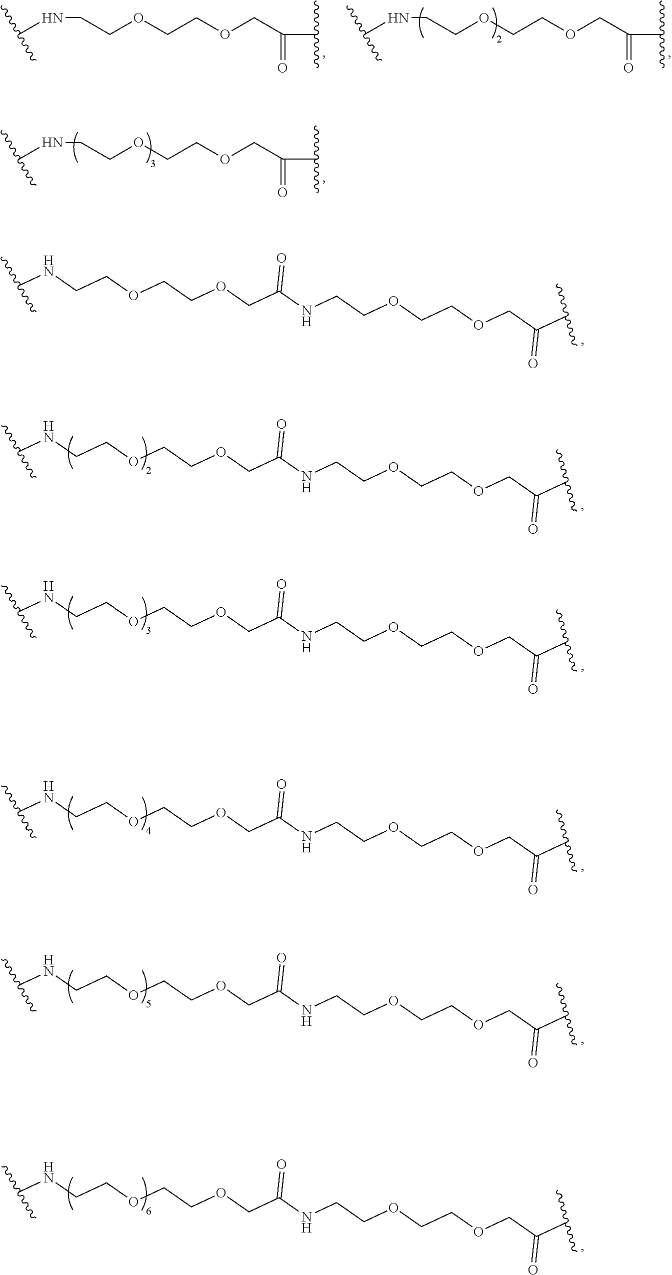

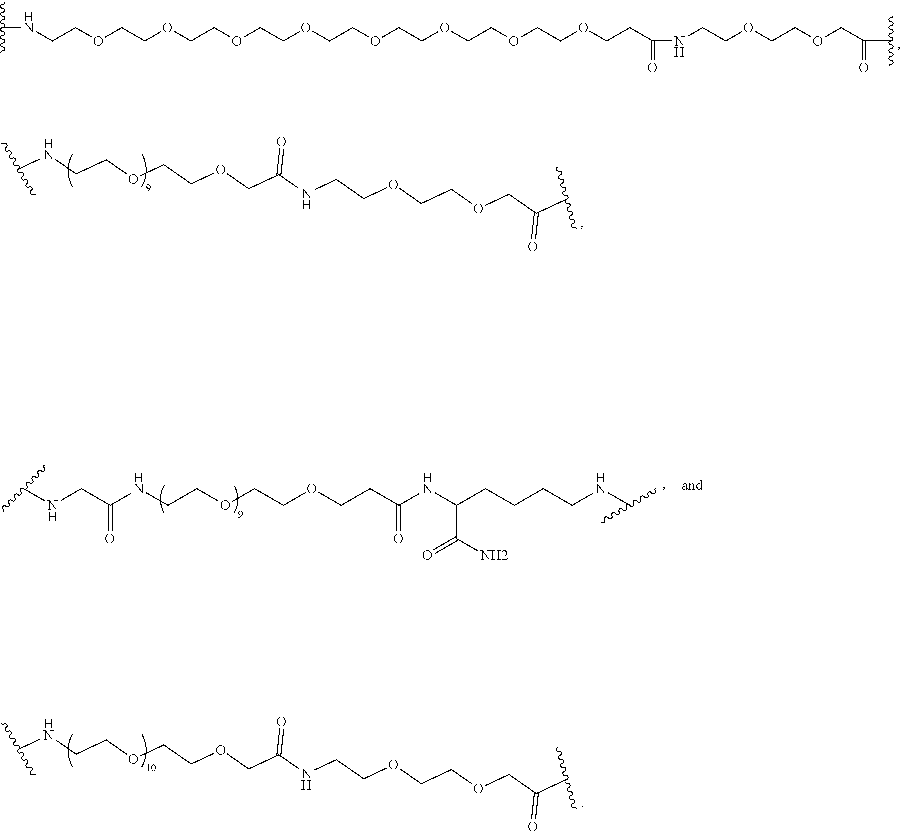

In some embodiments, the linker is a bond.

In some embodiments, a conjugate comprises a linker structure selected from Table 6

TABLE 6

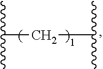

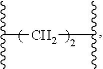

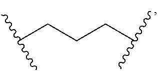

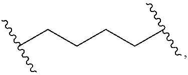

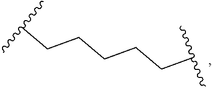

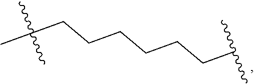

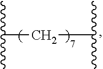

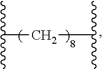

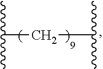

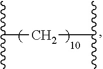

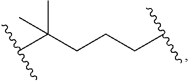

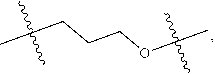

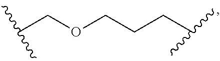

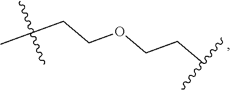

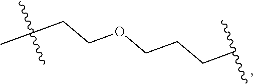

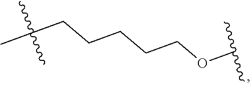

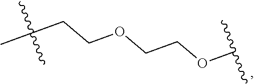

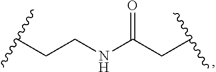

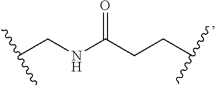

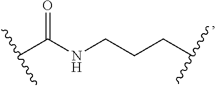

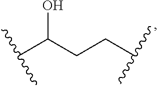

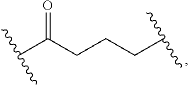

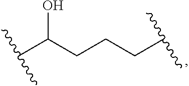

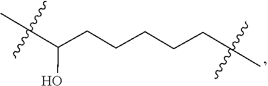

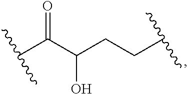

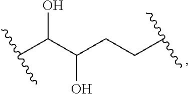

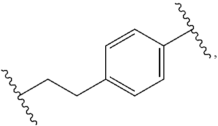

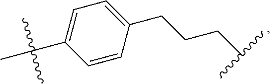

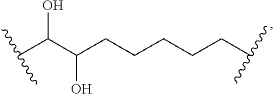

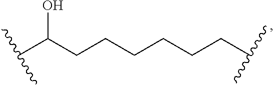

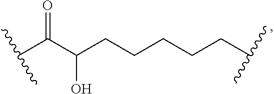

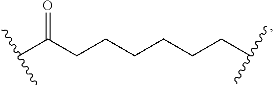

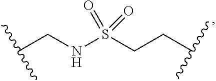

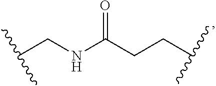

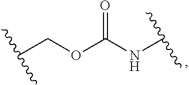

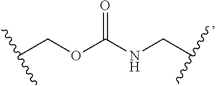

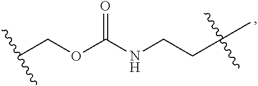

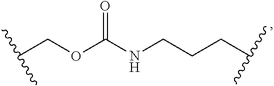

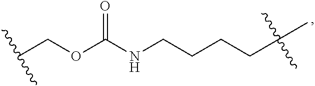

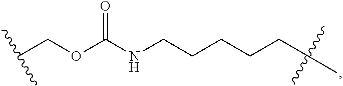

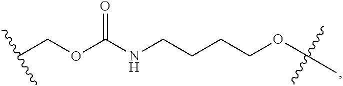

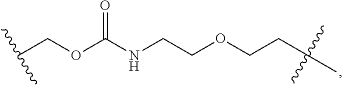

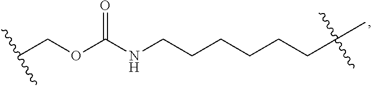

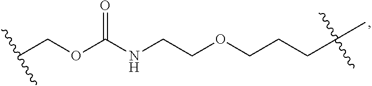

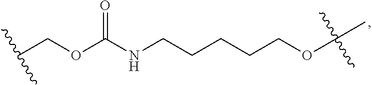

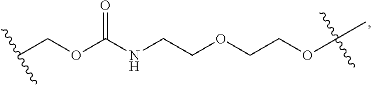

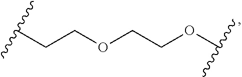

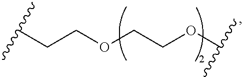

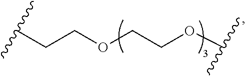

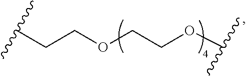

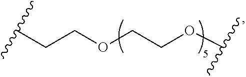

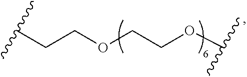

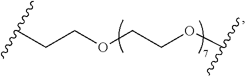

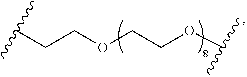

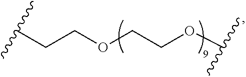

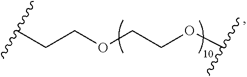

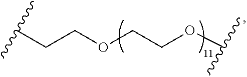

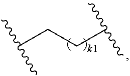

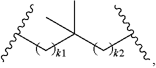

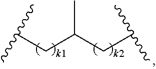

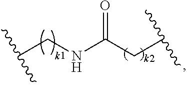

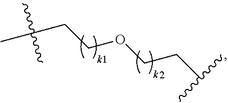

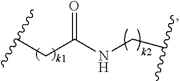

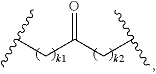

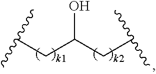

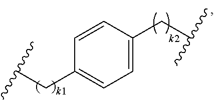

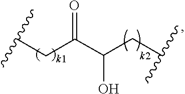

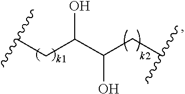

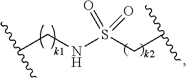

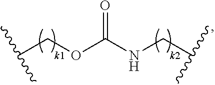

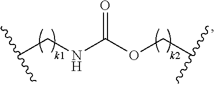

wherein each k1 and k2 is independently 0 or an integer selected from 1 to 20.

In some embodiments of Table 6, k1 is selected from 0-12. In some embodiments, k1 is 0. In some embodiments, k1 is 1. In some embodiments, k1 is 2. In some embodiments, k1 is 3. In some embodiments, k1 is 4. In some embodiments, k1 is 5. In some embodiments, k1 is 6. In some embodiments, k1 is 7. In some embodiments, k1 is 8. In some embodiments, k1 is 9. In some embodiments, k1 is 10. In some embodiments of Table 6, k2 is selected from 0-12. In some embodiments, k2 is 0. In some embodiments, k2 is 1. In some embodiments, k2 is 2. In some embodiments, k2 is 3. In some embodiments, k2 is 4. In some embodiments, k2 is 5. In some embodiments, k2 is 6. In some embodiments, k2 is 7. In some embodiments, k2 is 8. In some embodiments, k2 is 9. In some embodiments, k2 is 10.

### Metal Chelator

In one aspect, described herein are conjugates that comprise a metal chelator that is configured to bind with a radionuclide. The metal chelator can refer to a moiety of the conjugate that is configured to bind with a radionuclide. In some embodiments, a conjugate described herein comprises two or more independent metal chelators, e.g., 2, 3, 4, 5, or more metal chelators. In some embodiments, a conjugate described herein comprises two metal chelators, which can be the same or different. In some embodiments, a conjugate described herein comprises two or more metal chelators. In some embodiments, the conjugate comprises two radionuclides bound to the metal chelators. The metal chelator can be attached to the linker or the peptide through any suitable group/atom of the chelator.

In some embodiments, the metal chelator is capable of binding a radioactive atom. The binding can be direct, e.g., the metal chelator can make hydrogen bonds or electrostatic interactions with the radioactive atom. The binding can also be indirect. e.g., the metal chelator binds to a molecule that comprises a radioactive atom. In some embodiments, the metal chelator comprises, or is, a macrocycle. In some embodiments, the metal chelator comprises, or is, 2,2′,2″,2′″-(1,4,7,10-Tetraazacyclododecane-1,4,7,10-tetrayl)tetraacetic acid (DOTA) or 1,4,7-triazacyclononane-1,4,7-triacetic acid (NOTA). In some embodiments, the metal chelator comprises a macrocycle, e.g., a macrocycle comprising an O and/or a N, DOTA, NOTA, one or more amines, one or more ethers, one or more carboxylic acids, EDTA, DTPA, TETA, DO3A, PCTA, or desferrioxamine.

In some embodiments, the metal chelator comprises a plurality of amines. In some embodiments, the metal chelator includes 4 or more N, 4 or more carboxylic acid groups, or a combination thereof. In some embodiments, the metal chelator does not comprise S. In some embodiments, the metal chelator comprises a ring. In some embodiments, the ring comprises an O and/or an N. In some embodiments, the metal chelator is a ring that includes 3 or more N, 3 or more carboxylic acid groups, or a combination thereof. In some embodiments, the metal chelator is polydentate.

In some embodiments, a metal chelator described herein is selected from: DOT A, DOTA-GA, pBn-DOTA, pBn-SCN-DOTA, —NH2-DOTA, —NH2-DOTA-GA, p-NCS-Bn-DOTA-GA, p-NH2-Bn-oxo-DO3A, p-SCN-Bn-oxo-DO3A, NOTA, NODA-GA. NH2-NODA-GA, p-NCS-Bn-NODA-GA, p-NH2-Bn-NOTA, p-SCN-Bn-NOTA, NCS-MP-NODA, —NH2-MPAA-NODA, PCTA, p-NH2-Bn-PCTA, p-SCN-Bn-PCTA, p-SCN-Bn-HEHA, H2-MACROPA-NCS, H1-MACROPA, H2-MACROPA-NH2, H4-OCTAPA, tetra-(S, S, S, S)-Me-DOTA, tetra-(S, S, S, S)-Et-DOTA, tetra-(S, S, S, S)-iBu-DOTA, or maleimide-nBu-DOTA.

In some embodiments, a metal chelator described herein has a structure of

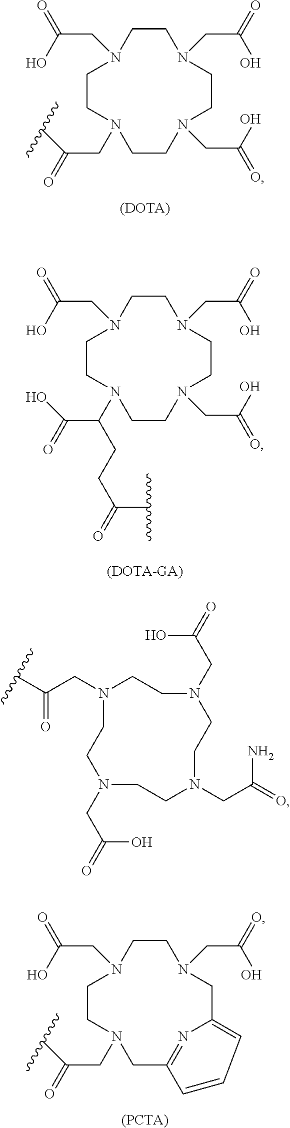

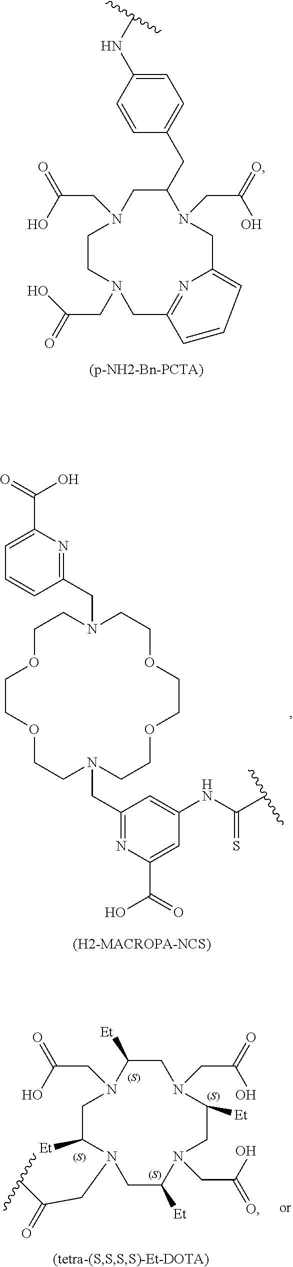

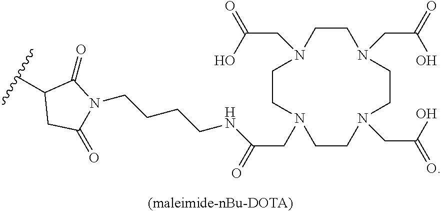

In some embodiments, a metal chelator described herein has a structure of

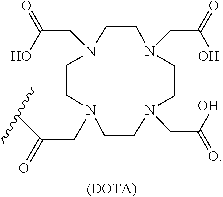

In some embodiments, a metal chelator described herein comprises a cyclic chelating agent. Exemplary cyclic chelating agents include, but are not limited to, AAZTA, BAT, BAT-TM, Crown, Cyclen, DO2A, CB-DO2A, DO3A, H3HP-DO3A, Oxo-DO3A, p-NH₂-Bn-Oxo-DO3A, DOTA. DOTA-3py, DOTA-PA, DOTA-GA, DOTA-4AMP, DOTA-2py, DOTA-1py, p-SCN-Bn-DOTA, CHX-A″-EDTA, MeO-DOTA-NCS EDTA, DOTAMAP. DOTAGA, DOTAGA-anhydride, DOTMA, DOTASA. DOTAM, DOTP, CB-Cyclam, TE2A, CB-TE2A, CB-TE2P, DM-TE2A, MM-TE2A, NOTA, NOTP, HEHA, HEHA-NCS, p-SCN-Bn-HEHA, DTPA, CHX-A″-DTPA, p-NH₂-Bn-CHX-A″-DTPA, p-SCN-DTPA, p-SCN-Bz-Mx-DTPA, 1B4M-DTPA, p-SCN-BnIB-DTPA, p-SCN-Bn-1B4M-DTPA, p-SCN-Bn-CHX-A″-DTPA, PEPA, p-SCN-Bn-PEPA, TETPA, DOTPA, DOTMP, DOTPM, t-Bu-calix[4]arene-tetracarboxylic acid, macropa, macropa-NCS, macropid, H3L¹, H3L^{X4}, Hrazapa, H₅decapa, bispa², H₄pypa, H₄octapa, H₄CHXoctapa, p-SCN-Bn-H₄octapa, p-SCN-Bn-H4octapa, TTHA, p-NO2-Bn-neunpa, H₄octox, H₂macropa, H₂bispa², H₄phospa, H₆phospa, p-SCN-Bn-H₆phospa. TETA, p-NO₂-Bn-TETA, TRAP. TPA. HBED, SHBED. HBED-CC, (HBED-CC)TFP, DMSA, DMPS, DHLA, lipoic acid, TGA, BAL, Bis-thioseminarabazones, p-SCN-NOTA, nNOTA, NODAGA. CB-TEIA1P, 3P-C-NETA-NCS, 3p-C-DEPA, 3P-C-DEPA-NCS, TCMC, PCTA, NODIA-Me, TACN, pycup1AlB, pycnp2A, THP, DEDPA, H₂DEDPA, p-SCN-Bn-H₂DEDPA, p-SCN-Bn-TCMC, motexafin, NTA, NOC, 3p-C-NETA, p-NH-Bn-TE3A, SarAr, DiAmSar, SarAr-NCS, AmBaSar, BaBaSar. TACN-TM. CP256. C-NE3TA, C-NE3TA-NCS. NODASA, NETA-monoamide, C-NETA, NOPO, BPCA, p-SCN-Bn-DFO, DFO-ChX-Mal, DFO, DFO-IAC, DFO-BAC, DiP-LICAM, EC, SBAD, BAPEN, TACIPYR, NEC-SP, L^{Py}, L1, L2, L3, and EuK-106. In some embodiments, the metal chelator is DOTA, TRITA, TETA. DOTA-MA. DO3A-HP, DOTMA. DOTA-pNB, DOTP, DOTMP, DOTEP, DOTMPE, F-DOTPME, DOTPP, DOTBzP, DOTA-monoamide, p-NCS-DOTA, p-NCS-PADOTA. BAT. DO3TMP-Monoamide, p-NCS-TRITA, NOTA, or CHX-A″-DTPA. In some embodiments, a metal chelator described herein comprises an acyclic chelating agent. Exemplary acyclic chelating agents include, but are not limited to, DTA, CyEDTA, EDTMP, DTPMP, DTPA, CyDTPA, Cy2DTPA, DTPA-MA, DTPA-BA, and BOPA. In some embodiments, a metal chelator described herein comprises DOTA, DOTP, DOTMA, DOTAM, DTPA, NTA, EDTA, DO3A, DO2A, NOC, NOTA, TETA. TACN, DiAmSar, CB-Cyclam, CB-TE2A, DOTA-4AMP, or NOTP. In some embodiments, a metal chelator described herein comprises H₄pypa, H₄octox, H₄octapa, p-NO₂-Bn-neunpa, p-SCN-Bn-H₄neunpa, TTHIA, ^{t}Buapypa-C7-NHS, H₄neunpa, H2macropa, HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA. TRAP, AAZTA, DATA″ THP, HEHA, or HBED.

In some embodiments, the metal chelator is DO3A. In some embodiments, the metal chelator is PEPA. In some embodiments, the metal chelator is EDTA. In some embodiments, the metal chelator is CHX-A″-DTPA. In some embodiments, the metal chelator is HEHA. In some embodiments, the metal chelator is DOTMP. In some embodiments, the metal chelator is t-Bu-calix[4]arene-tetracarboxylic acid. In some embodiments, the metal chelator is macropa. In some embodiments, the metal chelator is macropa-NCS. In some embodiments, the metal chelator is H₄pypa. In some embodiments, the metal chelator is H₄aoctapa. In some embodiments, the metal chelator is H₄CHXoctapa. In some embodiments, the metal chelator is DOTP. In some embodiments, the metal chelator is crown.

In some embodiments, the metal chelator is DOTA. In some embodiments, the metal chelator is a chiral derivative of DOTA. Exemplary chiral DOTA chelators are described in Dai et al., Nature Communications (2018) 9:857. In some embodiments, the metal chelator is 2,2′,2,2″-((2S558S,1 S)-2,5,8,11-tetramethyl-1,4,7,10-tetraazacyclododecane-1,4,7,10-tetrayl)tetraacetic acid. In some embodiments, the metal chelator has a structure of

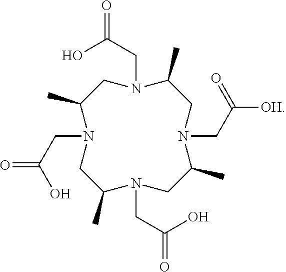 [SMILES: C[C@H]1CN(CC(=O)O)[C@@H](C)CN(CC(=O)O)[C@@H](C)CN(CC(=O)O)[C@@H](C)CN1CC(=O)O]

In some embodiments, the metal chelator is 2,2′,2″,2′-((2S,5S,8S,11S)-2,5,8,11-tetraethyl-1,4,7,10-tetraazacyclododecane-1,4,7,10-tetrayl)tetraacetic acid. In some embodiments, the metal chelator has a structure of

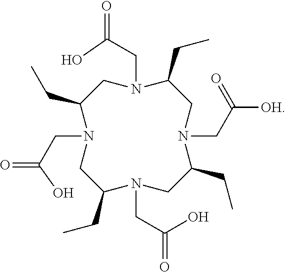 [SMILES: CC[C@H]1CN(CC(=O)O)[C@@H](CC)CN(CC(=O)O)[C@@H](CC)CN(CC(=O)O)[C@@H](CC)CN1CC(=O)O]

In some embodiments, the metal chelator has a structure of

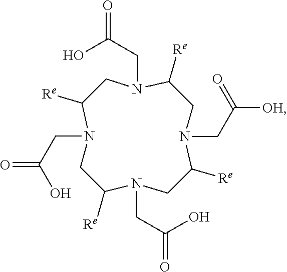

wherein each R^{e} is independently selected from hydrogen, alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylheterocycloalkyl, alkylaryl, alkylheteroaryl, or an amino acid side chain. In some embodiments,

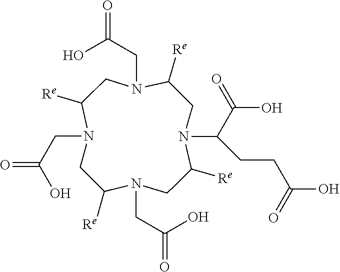

the metal chelator has a structure of wherein each R^{e} is independently selected from hydrogen alkyl, haloalkyl, hydroxyalkyl, aminoalkyl, heteroalkyl, alkenyl, alkynyl, cycloalkyl, heterocycloalkyl, aryl, heteroaryl, alkylcycloalkyl, alkylaryl, alkylheteroaryl, or an amino acid side chain.

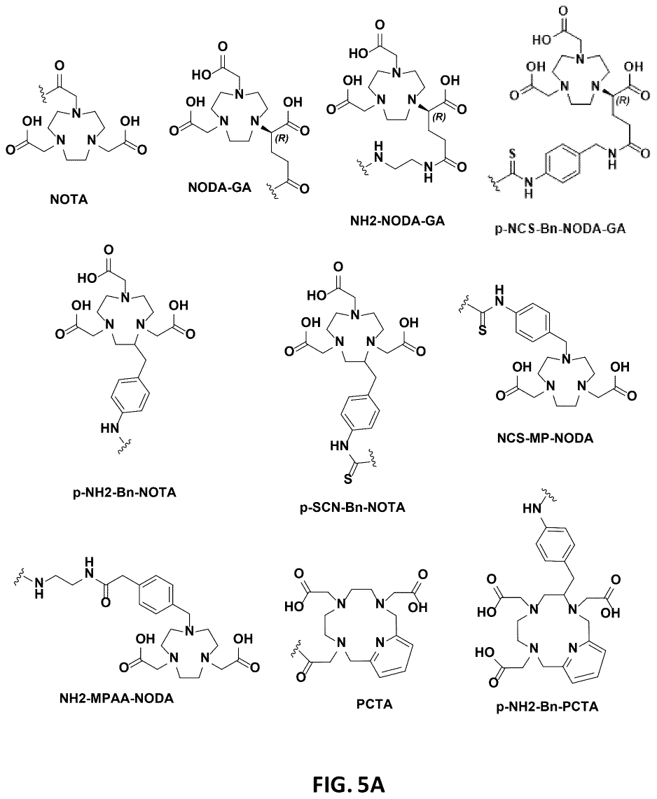

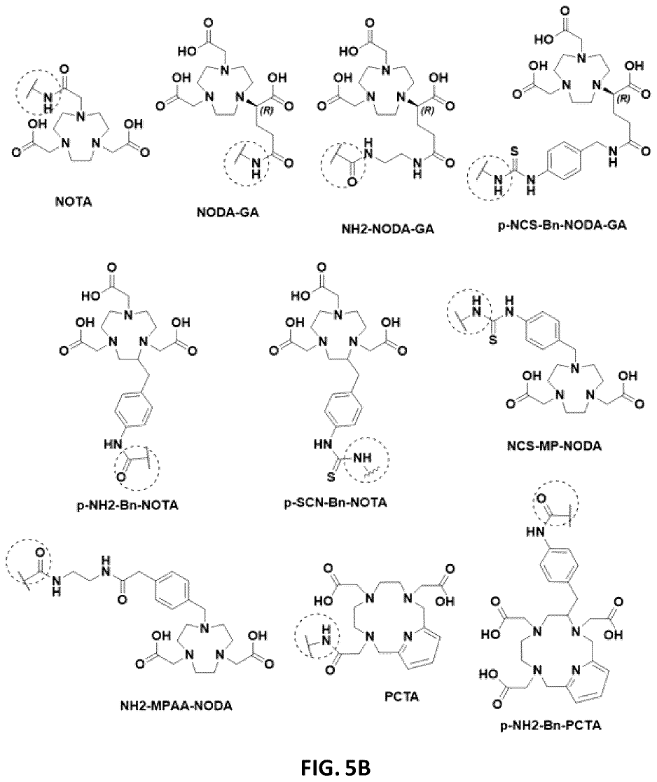

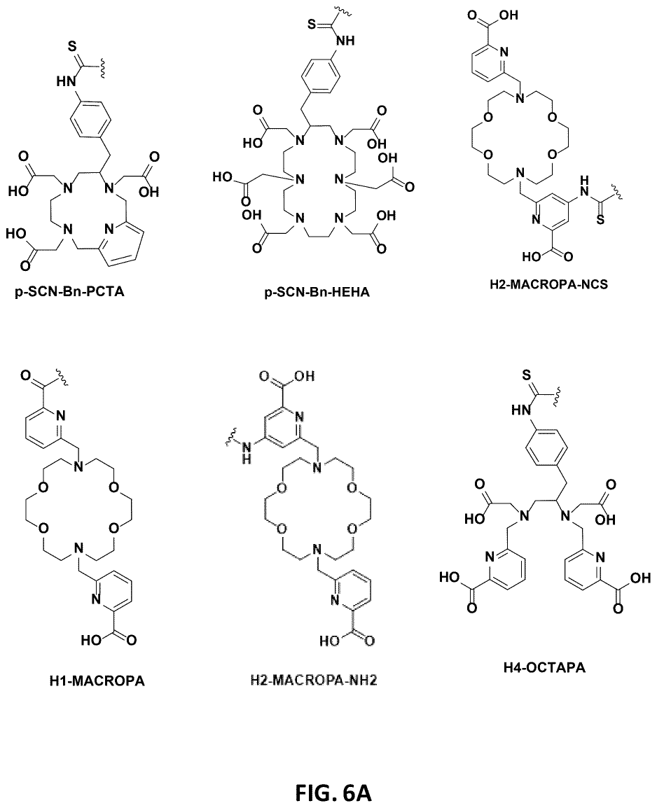

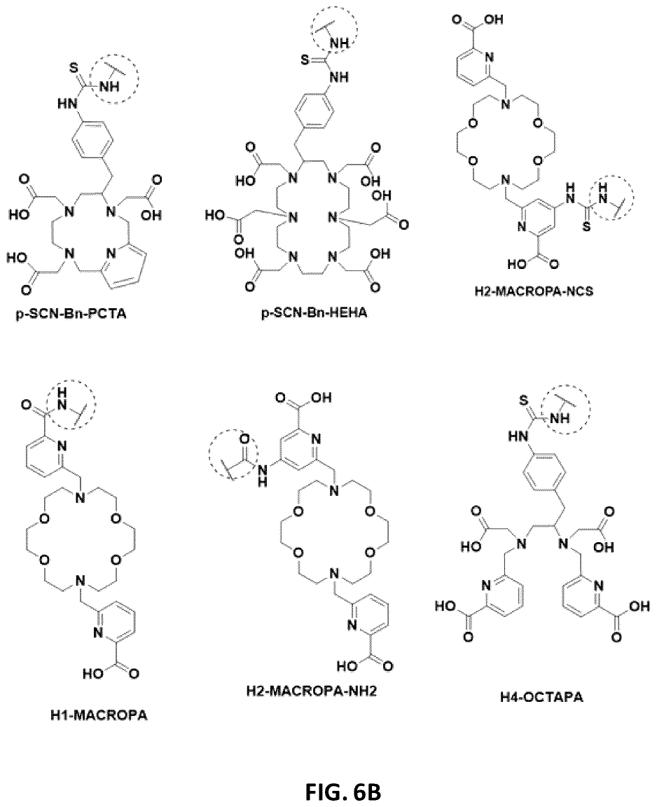

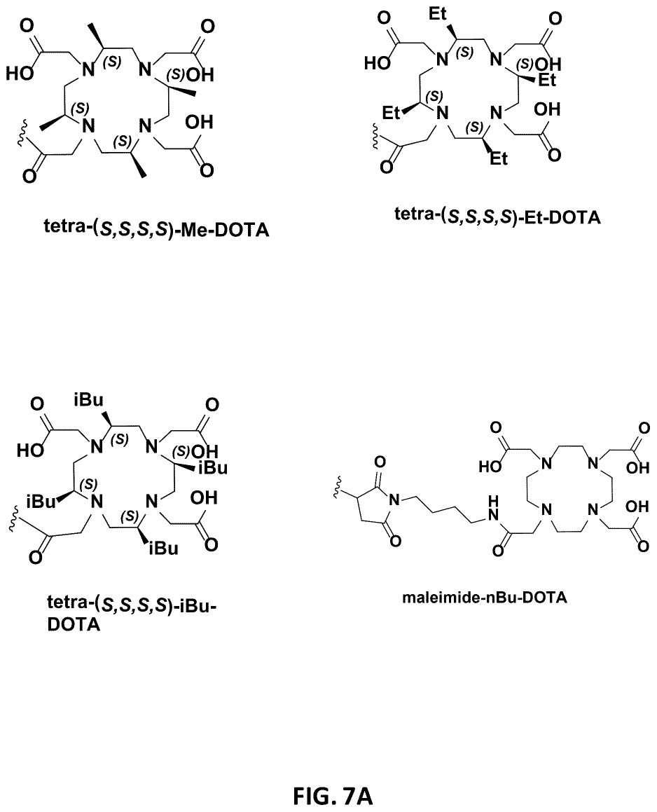

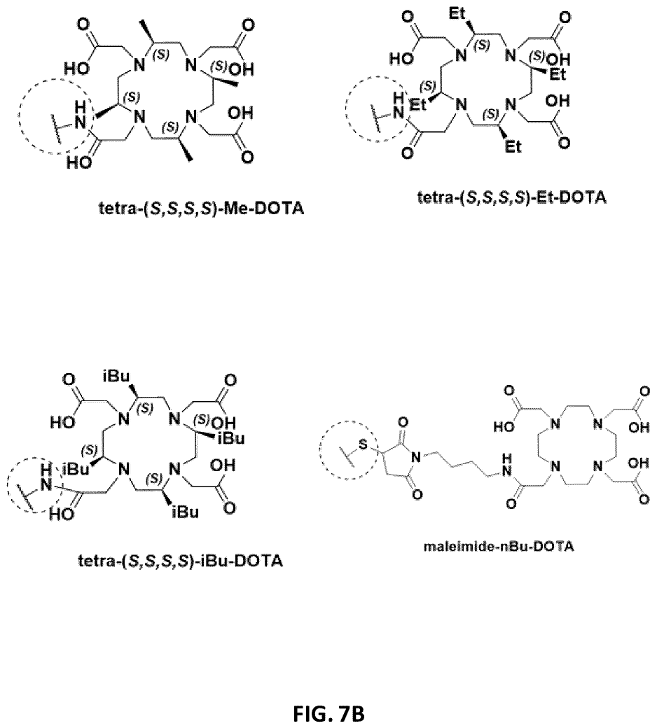

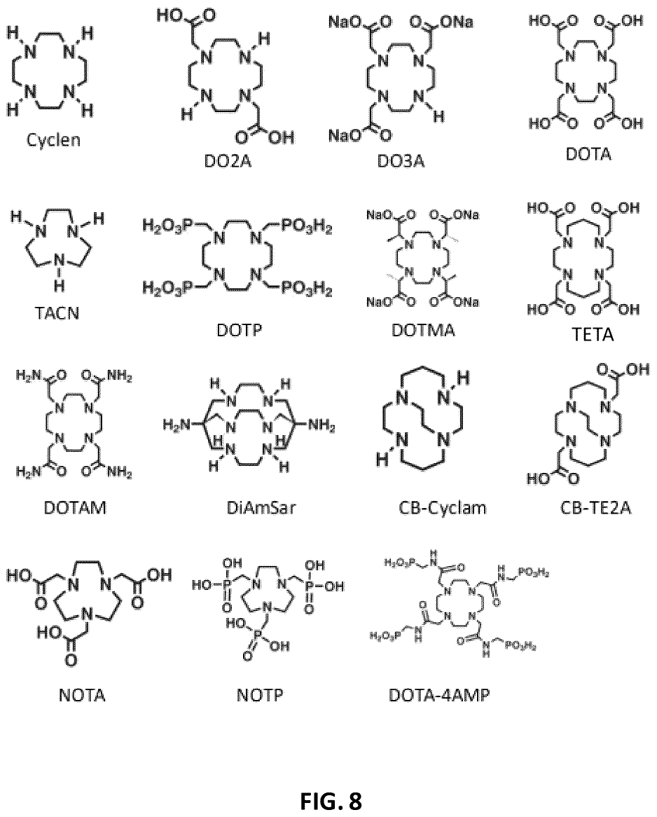

In some embodiments, the conjugate comprises DOTA. In some embodiments, the conjugate comprises a DOTA derivative such as p-SCN-Bn-DOTA, and MeO-DOTA-N-CS. In some embodiments, the conjugate comprises two independent metal chelators, and at least one or both are DOTA. The structures of some exemplary metal chelators are illustrated in FIGS. 8-22 (without showing the attachment points). Exemplary metal chelators are also illustrated in FIGS. 4A, 5A, 6A, and 7A (attachment point shown as a squiggly line) and FIGS. 4B, 5B, 6B and 7B (except that a part of the linker or the peptide covalently connected to the metal chelator is shown in the dashed circle). In some embodiments, a conjugate comprises a metal chelator of FIG. 4A. In some embodiments, a conjugate comprises a metal chelator of FIG. 5A. In some embodiments, a conjugate comprises a metal chelator of FIG. 6A. In some embodiments, a conjugate comprises a metal chelator of FIG. 7A. Exemplary metal chelators are further described in WO2012/174136; US20130183235A1; US20120219495A1; Ramogidaand et al., EJNMMI radiopharm, chem. 4, 21 (2019); Thiele et al., Cancer Biotherapy and Radiopharnaceuticals 2018; Li et al., Bioconjugate Chem. 2019, 30, 5, 1539-1553; and Baranyai et al., Eur. J. Inorg. Chem. 36-56 (2020), each of which is incorporated by reference in its entirety.

A metal chelator such as DOTA can interact with a radionuclide (e.g. ¹⁷⁷Lu or ²²⁵Ac) via one or more functional groups and/or atoms. For example, a metal chelator can interact with a radionuclide via nitrogen and/or oxygen atoms. As another example, a metal chelator can interact with a radionuclide via carbonyl, carboxylic acid, amino, and/or amide groups of the metal chelator. In some embodiments, the interaction of a metal chelator and a radionuclide of the conjugates disclosed herein can be illustrated as

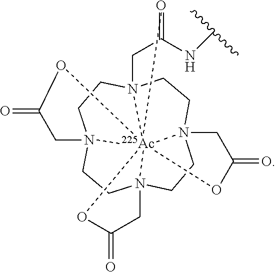

In some embodiments, the interaction of a metal chelator and a radionuclide of the conjugates disclosed herein can be illustrated as

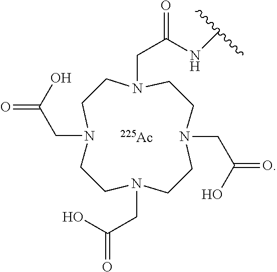

In some embodiments, the interaction of a metal chelator and a radionuclide of the conjugates disclosed herein can be illustrated as

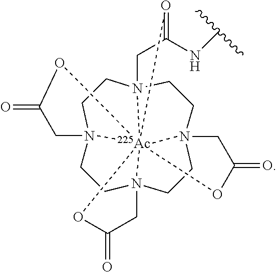

In some embodiments, the interaction of a metal chelator and a radionuclide of the conjugates disclosed herein can be illustrated as

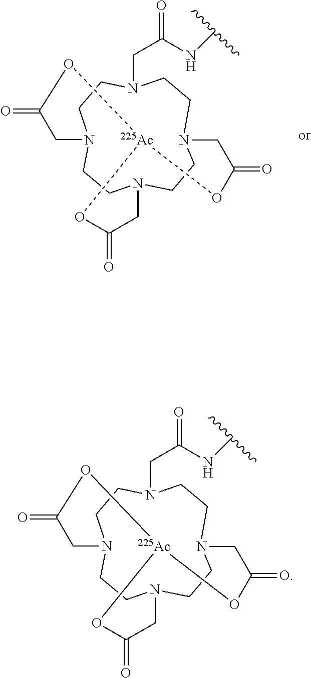

In some embodiments, the interaction of a metal chelator and a radionuclide of the conjugates disclosed herein can be illustrated as

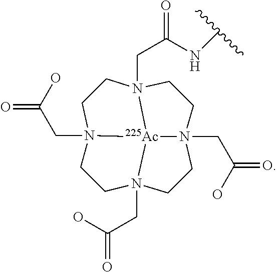

In some embodiments, the interaction of a metal chelator and a radionuclide of the conjugates disclosed herein can be illustrated as

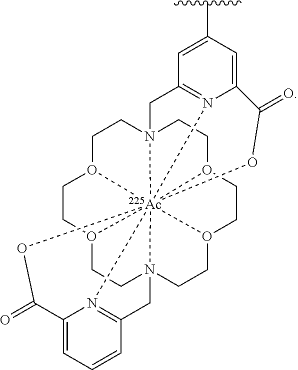

In some embodiments, the radionuclide exists in a positive oxidation state e.g., ²²⁵Ac³⁺, ¹⁷⁷Lu³⁺. In some embodiments, for example in certain aqueous conditions, the radionuclide exists in a salt form, e.g., as ²²⁵Ac³⁺, ¹⁷⁷Lu³⁺. In some embodiments, for example in certain acidic aqueous conditions, the radionuclide exists in a salt form, e.g., as ²²⁵Ac³⁺, ¹⁷⁷Lu³⁺. In some embodiments, the conjugate is in a salt form. In some embodiments, one or more of the carboxylic acid groups of the conjugate may exist as carboxylate anions. In some embodiments, one or more of the carboxylate anions of the conjugate may coordinate to the radionuclide. A person of ordinary skill would appreciate that the dissociation of an acid can depend on the pH value of the environment and its pK value. Accordingly, in some embodiments, a conjugate described herein can exist in a completely ionized, partially ionized or non-ionized form.

### Radionuclide

In one aspect, disclosed herein are radiopharmaceutical conjugates comprising a radionuclide. In some embodiments, the radionuclide is chelated or bound to a metal chelator. In some embodiments, the radionuclide is covalently bound to the conjugate. Generally, the type of radionuclide used in a therapeutic radiopharmaceutical can be tailored to the specific type of cancer, the type of targeting moiety (e.g., binding peptide), etc. Radionuclides that undergo α-decay produce particles composed of two neutrons and two protons, and radionuclides that undergo β-decay emit energetic electrons from their nuclei. Some radionuclides can also undergo electron capture and emit auger electrons. In some embodiments, the conjugate comprises an alpha particle-emitting radionuclide. Alpha radiation can cause direct, irreparable double-strand dna breaks compared with gamma and beta radiation, which can cause single-stranded breaks via indirect dna damage. The range of these particles in tissue and the half-life of the radionuclide can also be considered in designing the radiopharmaceutical conjugate. Table 7 below illustrates some properties of exemplary radionuclides.

TABLE 7
Exemplary radionuclides

| | | Exemplary application--Therapeutics |
|---|---|---|
| Radionuclide | Emission/Decay | (Tx) or Diagnostics (Dx) |
| Ac-225 | Alpha | Tx |
| As-70 | Positron | Dx |
| As-71 | Positron | Dx |
| As-72 | Positron | Dx |
| As-73 | Electron Capture | Dx |
| As-74 | Positron | Dx |
| As-76 | Beta | Tx |
| As-77 | Beta | Tx |
| At-211 | Alpha | Tx |
| Bi-209 | Alpha | Tx |
| Bi-212 | Alpha | Tx |
| Bi-213 | Alpha | Tx |
| Br-76 | Positron | Dx |
| Ce-134 | Electron Capture | Dx |
| Cu-61 | Positron | Dx |
| Cu-62 | Positron | Dx |
| Cu-64 | Positron | Dx |
| Cu-67 | Beta | Tx |
| Fr-223 | Alpha | Tx |
| Ga-68 | Positron | Dx |
| Gd-148 | Alpha | Tx |
| Ho-166 | Beta/gamma | Tx/Dx |
| I-123 | Electron Capture | Dx |
| I-124 | Positron | Dx |
| I-125 | Electron Capture | Dx |
| I-131 | Beta | Tx |
| In-111 | Beta/Electron | Tx/Dx |
| | Capture | |
| Lu-177 | Beta | Tx |
| Pb-212 | Alpha | Tx |
| Pm-153 | Beta | Tx |
| Po-213 | Alpha | Tx |
| Ra-223 | Alpha | Tx |
| Ra-224 | Alpha | Tx |
| Rh-105 | Beta | Tx |
| Sm-153 | Beta | Tx |
| Tb-149 | Alpha | Tx |
| Tb-152 | Positron | Dx |
| Tb-161 | Beta | Tx/Dx |
| Tc-99m | Gamma/Isomeric | Dx |
| | Transition | |
| Th-227 | Alpha | Tx |
| Th-229 | Alpha | Tx |
| Tm-167 | Beta/Electron | Tx |
| | Capture | |
| Y-90 | Beta | Tx |
| Yb-175 | Beta | Tx |
| Zr-89 | Positron | Dx |

In some embodiments, the radiopharmaceutical conjugate described herein comprises a radionuclide selected from Table 7.

In some embodiments, the radiopharmaceutical conjugate described herein comprises one or more independent radionuclides. In some embodiments, the radiopharmaceutical conjugate comprises two radionuclides. In some embodiments, each of the one or more radionuclides is bound to the metal chelator of the radiopharmaceutical conjugate. In some embodiments, two radionuclides of the radiopharmaceutical conjugate are bound to the same metal chelator. In some embodiments, two radionuclides of the radiopharmaceutical conjugate are bound to two independent metal chelators. In some embodiments, each of the one or more radionuclides is an alpha particle-emitting radionuclide.

In some embodiments, the radiopharmaceutical conjugate described herein comprises an alpha particle-emitting radionuclide. In some embodiments, the radiopharmaceutical conjugate comprises an alpha-particle emitting radionuclide bound to the metal chelator. In some embodiments, the alpha particle-emitting radionuclide is actinium-225 (²²⁵Ac), radium-223 (²²³Ra), radium-224 (²²⁴Ra), bismuth-209 (²⁰⁹Bi), bismuth-213 (²¹³Bi), gadolinium-148 (¹⁴⁸Gd), terbium-149 (¹⁴⁹Tb), polonium-213 (²¹³Po), francium-223 (²²³Fr), thoriunm-227 (²²⁷Th), thorium-229 (²²⁹Th), or lead-212 (²¹²Bb). In some embodiments, the alpha particle-emitting radionuclide is selected from ²²⁵Ac, ²²³Ra, ²⁰⁹Bi, ²¹³Bi, ¹⁴³Gd, ¹⁴⁹Tb, ²¹³Po, ²²³Fr, ²²⁷Th, ²²⁹Th. and ²¹²Pb. In some embodiments, the alpha particle-emitting radionuclide is ²²⁵Ac. In some embodiments, the alpha particle-emitting radionuclide is ²¹³Bi. In some embodiments, the alpha particle-emitting radionuclide is ²¹²Bi. In some embodiments, the alpha particle-emitting radionuclide is ²¹²Pb. In some embodiments, the alpha particle-emitting radionuclide is ²²⁴Ra. In some embodiments, the alpha particle-emitting radionuclide is ²²³Ra. In some embodiments, the alpha particle-emitting radionuclide is ²²⁷Th. In some embodiments, the alpha particle-emitting radionuclide is ¹⁴⁹Tb. In some embodiments, the conjugate comprises ²²⁵Ac. In some embodiments, the conjugate comprises two ²²⁵Ac radionuclides. In some embodiments, the radionuclide is no-carrier added (i.e., non-carrier-added or n.c.a.)¹⁷⁷Lu. In some embodiments, the radionuclide is no-carrier added (i.e., non-carrier-added or n.c.a.) ²²⁵Ac. In some embodiments, the radionuclide is ¹⁷⁷Lu free of long-lived radioactive contaminants and byproducts. In some embodiments, the conjugate comprises two ¹⁷⁷Lu radionuclides. In some embodiments, the radionuclide is a non-carrier-added radionuclide. In some embodiments, the radionuclide is a pseudo-radiometal. In some embodiments, the pseudo-radiometal is aluminum -[¹⁸F]fluoride ([¹⁸F]AIF) complex.

In some embodiments, the radiopharmaceutical conjugate described herein comprises a radionuclide selected from ⁶²Cu, ⁶⁴Cu, ⁶⁷Cu, ⁹⁰Y, ¹⁰⁹Pd, ¹¹¹Ag, ¹³⁴Ce, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ¹⁵³Pm, ⁴⁴Sc, ⁴⁷Sc, ²¹³Po, ²²²Pb, ²¹³Bi, ²¹²Bi, ²¹³Bi, ²²⁵Ac, ^{117m}Sn, ⁶⁷Ga, ¹⁴⁹Tb, ¹⁵²Tb, ¹⁶⁷Tm, ¹⁷⁵Yb, ²²³Ra ²²³Fr ²²⁷Th, ²²⁹Th, ²⁰¹TI, ¹⁴⁸Gd, ¹⁶⁰Gd, ¹⁴⁸Nd, ⁸⁹Sr, and ⁸⁹Zr. In some embodiments, the radionuclide is selected from ⁶²Cu, ⁶⁴Cu, ⁶⁷Cu, ⁶⁸Ga, ⁸⁹Zr, ⁹⁰Y, ^{99m}Tc, ¹⁰⁵Rh, ¹¹¹In, ¹³⁴Ce, ¹⁴⁸Gd ¹⁴⁹Tb, ¹⁵²Tb, ¹⁵³Pm ¹⁶⁷Tm, ¹⁷⁵Yb, ¹⁷⁷Lu, ²⁰⁹Bi, ²¹²Pb, ²¹³Po, ²¹³Bi, ²²³Ra, ²²³Fr, ²²⁷Th, ²²⁵Ac, and ²²⁹Th. In some embodiments, the radionuclide is ²²⁵Ac. In some embodiments, the radionuclide is a decay daughter of ²²⁵Ac such as ²²¹Fr. ²¹⁷At, ²¹³Bi, ²¹³Po ²⁰⁹TI, ²⁰⁹Pb, or ²⁰⁹Bi. In some embodiments, the radiopharmaceutical conjugate comprises two ²²⁵Ac radionuclides. In some embodiments, the radionuclide is ¹⁷⁷Lu. In some embodiments, the radiopharmaceutical conjugate comprises two ¹⁷⁷Lu radionuclides.

In some embodiments, the radiopharmaceutical conjugate described herein comprises a beta particle-emitting radionuclide. In some embodiments, the radiopharmaceutical conjugate comprises a beta particle-emitting radionuclide bound to the metal chelator. In some embodiments, the beta particle-emitting radionuclide is copper-67, rhodium-105, ytterbium-175, thulium-167, promethium-153, yttrium-90, samarium-153, or lutetium-177. In some embodiments, the beta particle emitting radionuclide is copper-67, yttrium-90, samarium-153, or lutetium-177. In some embodiments, the beta particle emitting radionuclide is lutetium-177.

In some embodiments, the radiopharmaceutical conjugate described herein comprises a gamma particle-emitting radionuclide. In some embodiments, the radiopharmaceutical conjugate comprises a gamma particle-emitting radionuclide bound to the metal chelator. In some embodiments, the gamma particle-emitting radionuclide is indium-1l or tin-117m.

In some embodiments, the radiopharmaceutical conjugate described herein comprises a positron particle-emitting radionuclide. In some embodiments, the radiopharmaceutical conjugate comprises a positron particle-emitting radionuclide bound to the metal chelator. In some embodiments, the positron-emitting radionuclide is gallium-68, copper-61, copper-62, copper-64, zirconium-89, or terbium-152. In some embodiments, the radionuclide is zirconium-89. In some embodiments, the radionuclide is gallium-68.

In some embodiments, a conjugate described herein comprises a radionuclide suitable for imaging or diagnostic purposes. In some embodiments, the radionuclide suitable for imaging is selected from ⁶²Cu, ⁶⁴Cu, ⁸⁹Zr, ¹³⁴Ce, ¹⁵²Tb, ⁶⁸Ga, ¹¹¹In, and ^{99m}Tc. In some embodiments, the radionuclide is suitable PET imaging. In some embodiments, the radionuclide suitable for PET imaging is selected from ⁶²Cu, ⁶⁴Cu, ⁸⁹Zr, ¹³⁴Ce, ¹⁵²Tb, and ⁶⁸Ga. In some embodiments, the radionuclide is suitable for SPECT imaging. In some embodiments, the radionuclide suitable for SPECT imaging is selected from ¹¹¹In and ^{99m}Tc.

In some embodiments, radiopharmaceutical conjugates described herein do not contain any hot radionuclide, i.e., a cold conjugate. For example, in some cases, a radionuclide can be replaced with a surrogate (e.g., ²²⁵Ac replaced with lanthanum) for testing and experimental purposes. In some embodiments, hot lutetium (Lu-177) is replaced with a cold lutetium (Lu-175).

In some embodiments, a radiopharmaceutical conjugate described herein comprises a covalently bound radionuclide and optionally a linker. In some embodiments, the linker can comprise a residualizing agent or a non-residualizing agent. A radionuclide can be attached to a peptide or a linker through a residualizing agent or a non-residualizing agent. In some embodiments, the radionuclide is covalently attached to the peptide or the linker through a residualizing agent. In some embodiments, the residualizing agent is SGMIB or SIPC. In some embodiments, the radionuclide is covalently attached to the peptide or the linker through a residualizing agent. In some embodiments, the non-residualizing agent is N-succinimidyl-4-iodobenzoate (PIB). In some embodiments, a radionuclide is covalently bound to the residualizing agent or the non-residualizing agent. In some embodiments, the radionuclide is covalently bound to the residualizing agent. Procedures and methods for synthesis of covalently bound residualizing agents are described in U.S. Pat. No. 9,839,704 which is herein incorporated by reference in its entirety.

In some embodiments, the residualizing agent is a tetrapeptide IMP-R4. IMP-R4 can be represented as MCC-Lys(MCC)-Lys(Z)-d-Tyr-d-Lys(X)-OH, where MCC is 4-(N-maleimidomethyl)-cyclohexane-1-carbonyl and Z is 1-((4-thiocarbonylamnino)benzyl)-DTPA. In some embodiments, the radionuclide¹³¹I is linked to linker or peptide via ¹³¹I-IMP-R. In some embodiments, the residualizing agent is a tetrapeptide IMP-R3. In some embodiments, the residualizing agent is a tetrapeptide IMP-R5. In some embodiments, the residualizing agent is a tetrapeptide IMP-R6. In some embodiments, the residualizing agent is a tetrapeptide IMP-R7. In some embodiments, the residualizing agent is a tetrapeptide IMP-R^{a}. Exemplary residualizing and non-residualizing agents are further illustrated in Stein R, et al. Improved iodine radiolabels fbr monoclonal antibody therapy, Cancer Res. 2003; 63:111-118: Reist C J, et al. Radioiodination of internalizing monoclonal antibodies using N-succinimidyl-S-iodo-3-pyridinecarboxylate, Cancer Res. 1996; 56:4970-4977; Ali S A, et al. Improving the tumor retention of radioiodinated antibody: aryl carbohydrate adducts. Cancer Res. 1990; 50(suppl):783s-788s; and Serengulam V. Govindan, et al., Clinical-Scale Radiolabeling of a Humanized Anticarcinoembryonic Antigen Monoclonal Antibody, hMN-14, with Residualizing 131I for Use in Radioimmunotherapy, Journal of Nuclear Medicine January 2005, 46 (1) 153-159.

In some embodiments of a radiopharmaceutical conjugate of the present disclosure, the conjugate comprises a covalently bound radionuclide, designated as R*, wherein R* is connected to the rest of the conjugate via a structure of

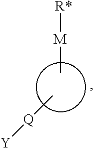

wherein

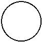

is a cycloalkyl, heterocycloalkyl, aryl, or heteroaryl ring, each of which is optionally
substituted;
Y is a charged group;
M and Q are chemical linking moieties; and
R R* is a covalently bound radionuclide (e.g., ¹⁸F, ¹²³I, ¹²⁴I, ¹²⁵I, and ²¹¹At).

In some embodiments,

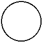

is phenyl.

In some embodiments. M and Q is each independently bond, C₁₋₂alkylene, C₂₋₂₀ alkenylene, C₁₋₂₀ alkynylene, or C₁₋₂₀ heteroalkylene, wherein the alkylene, alkenylene, alkynylene, and heteroalkylene are optionally substituted with C₁₋₈ alkyl or C₃₋₇ cycloalkyl. In some embodiments. M is a bond. In some embodiments, Q is a bond or C₁ alkylene.

In some embodiments, Y is a guanidino group.

In some embodiments.

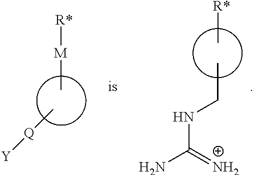

In some embodiments,

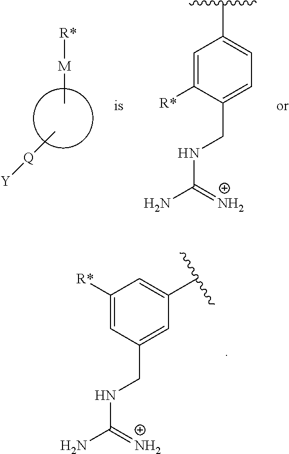

In some embodiments,

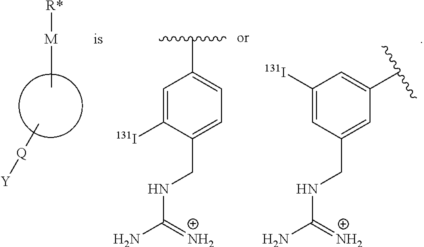

In some embodiments R* is connected through the rest of the conjugate via a linker of the following structures

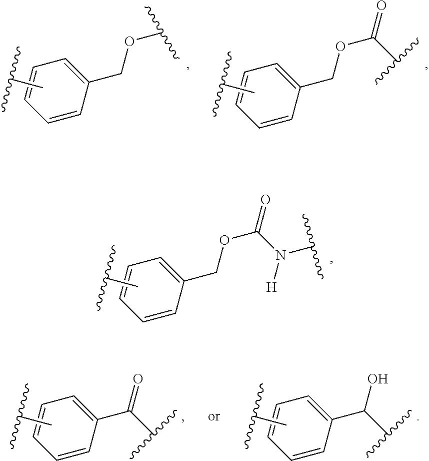

In some embodiments, a linker of the present disclosure, or one of L, L¹, L², L³, L⁴ or L⁵, comprises a structure selected from the group consisting of

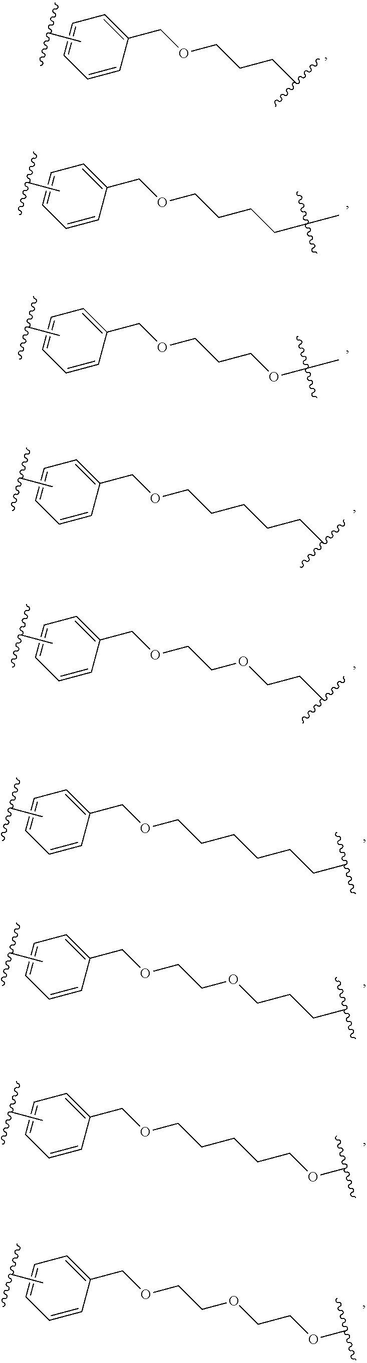

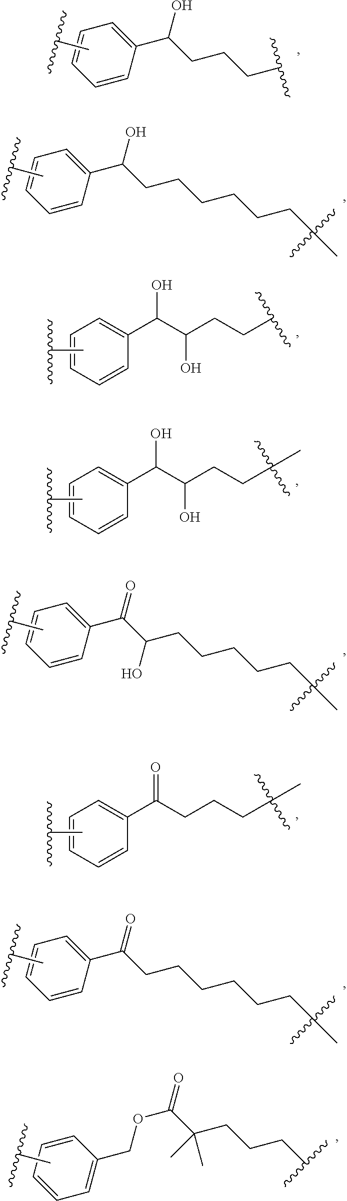

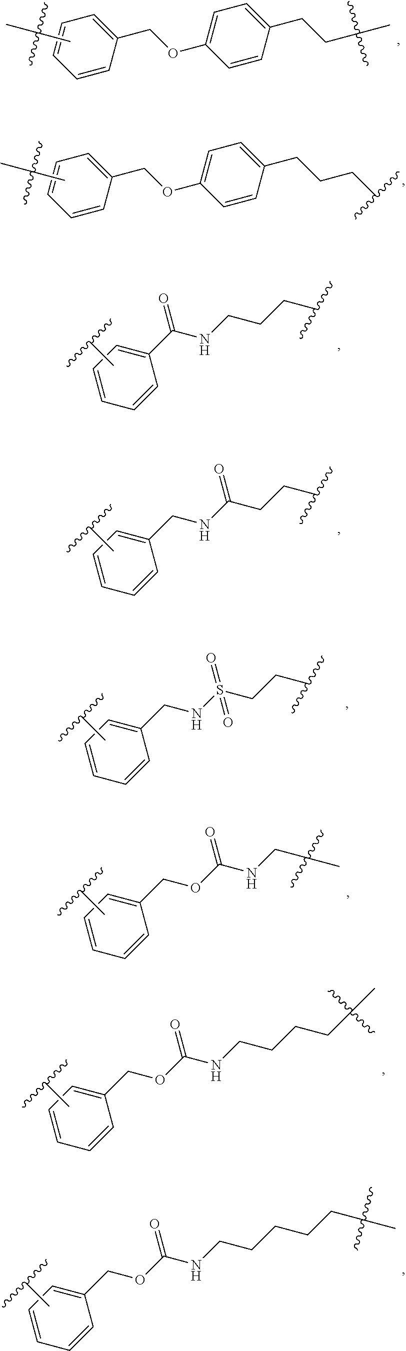

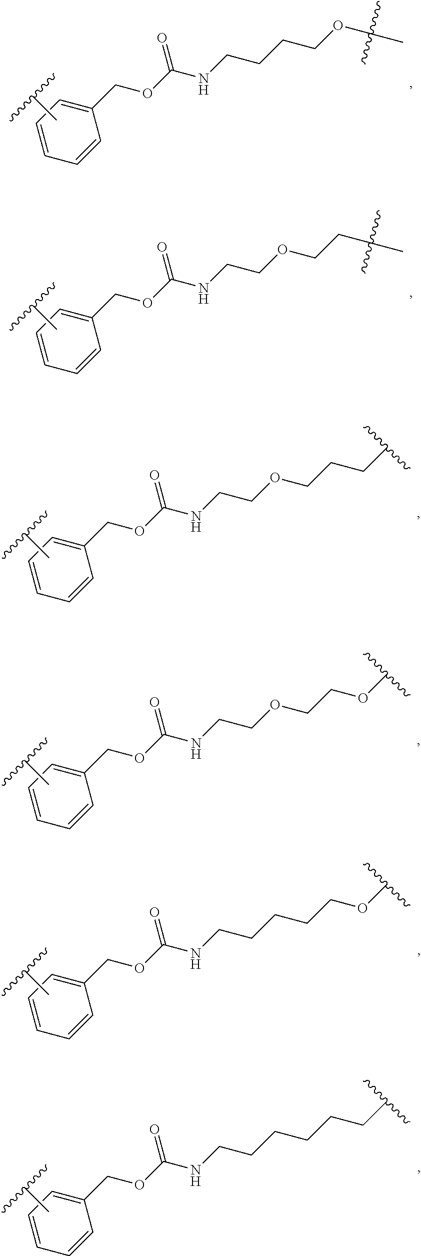

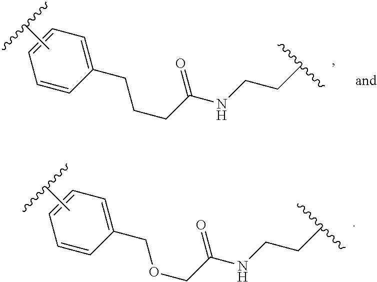

In some embodiments, the covalent radionuclide R* is connected to the phenyl.

In some embodiments, R* is selected from a radionuclide in Table 7. In some embodiments, the radionuclide is selected from fluorine-18 (¹⁸F), iodine-131 (¹³¹I), inosine-123 (¹²³I), iodine-124 (¹²⁴I), iodine-125 (¹²⁵I) or astatine-211 (²²¹At). In some embodiments, R* is iodine-131 (¹³¹I) or astatine-211 (221At). In some embodiments, the radionuclide is ¹³¹I. In some embodiments, the radionuclide is ¹²⁴I. In some embodiments, the radionuclide is ¹²⁵I. In some embodiments, the radionuclide is ²²¹At.

In some embodiments, a conjugate comprises a structure represented by Formula (Va), Formula (Vb), Formula (Ve, Formula (Vd), or Formula (Ve):

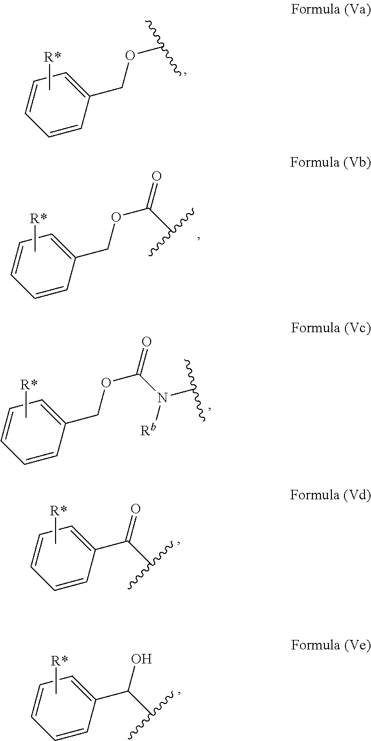

wherein,
R* is a covalently bound radionuclide (e.g., such as fluorine-18 (¹⁸F), iodine-131 (¹³¹I), iodine-123 (¹²³I), iodine-124 (¹²⁴I), iodine-125 (¹²⁵I), or astatine-211 (²²¹At));
R^{b} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃-C₈cycloalkyl, C₂-C₉heterocycloalkyl, aryl, or heteroaryl, wherein each of the alkyl, heteroalkyl, alkenyl, alkynyl, cycloalkyl, heterocycloalkyl, aryl, or heteroaryl is optionally substituted (e.g., optionally substituted with one or more R as described herein); and
L is C₁-C₆alkylene or C₁-C₆heteroalkylene, each of which is optionally substituted.

As illustrated above, Formula (Va), Formula (Vb), Formula (Vc), Formula (Vd), and Formula (Ve) can comprise all or a part of a linker and the radionuclide R*.

In some embodiments of Formula (Va), Formula (Vb). Formula (Ye). Formula (Vd), or Formula (Ve), the alkyl, haloalkyl, hydroxyalkyl, aminoalkyl, heteroalkyl, alkenyl, alkynyl, cycloalkyl, heterocycloalkyl, aryl, or heteroaryl are independently optionally substituted by one or more halogen, amino, —OH, —NO₂, oxo, —CN, C₁₋₃ alkoxyl, C₁₋₃ alkyl and C₁₋₃ haloalkyl. In some embodiments. R^{b} is hydrogen. In some embodiments, R^{b} is C₁-C₄alkyl. In some embodiments, R^{a} is C₁-C₄cycloalkyl. In some embodiments, R* is ¹³¹I.

In some embodiments, a structure represented in Formula (Va), Formula (Vb), Formula (Vc), Formula (Vd), and Formula (Ve) is selected from the following:

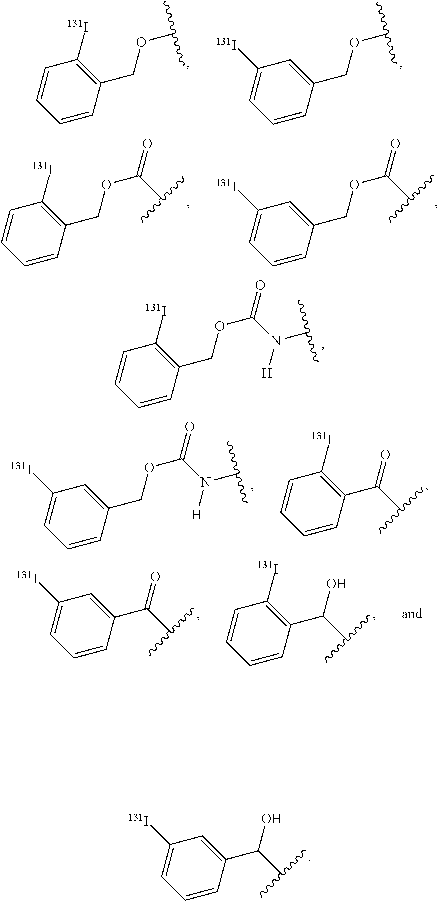

In some embodiments, a conjugate comprises a linker structure selected from:

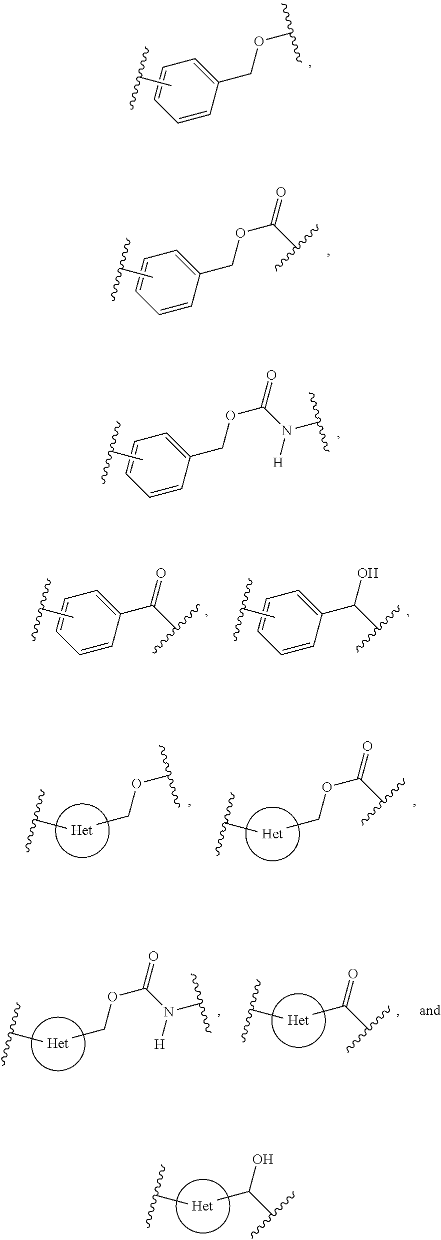

wherein Het is a 5-6 membered heteroaryl ring containing 1-3 heteroatoms independently selected from N, S, and O. In some embodiments, Het is pyridinyl or pyrimidinyl.

In some embodiments, a conjugate comprises a linker structure selected from:

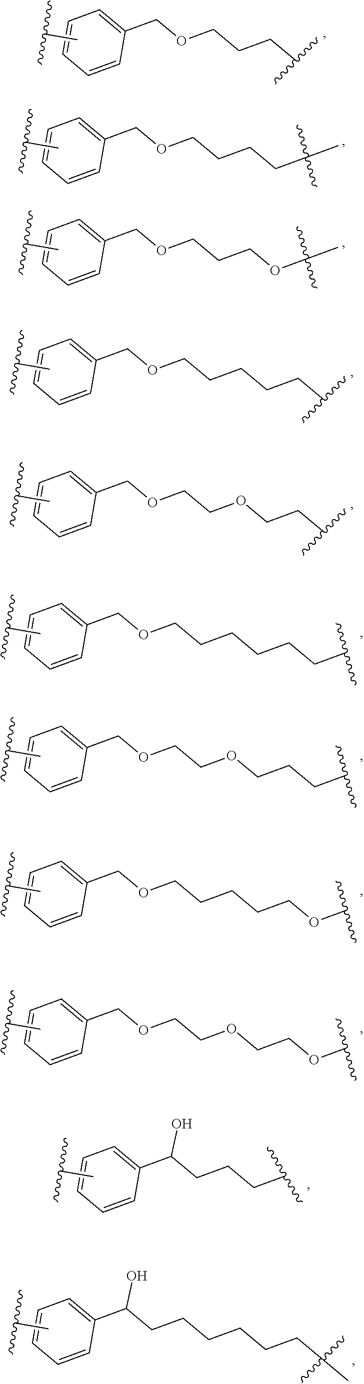

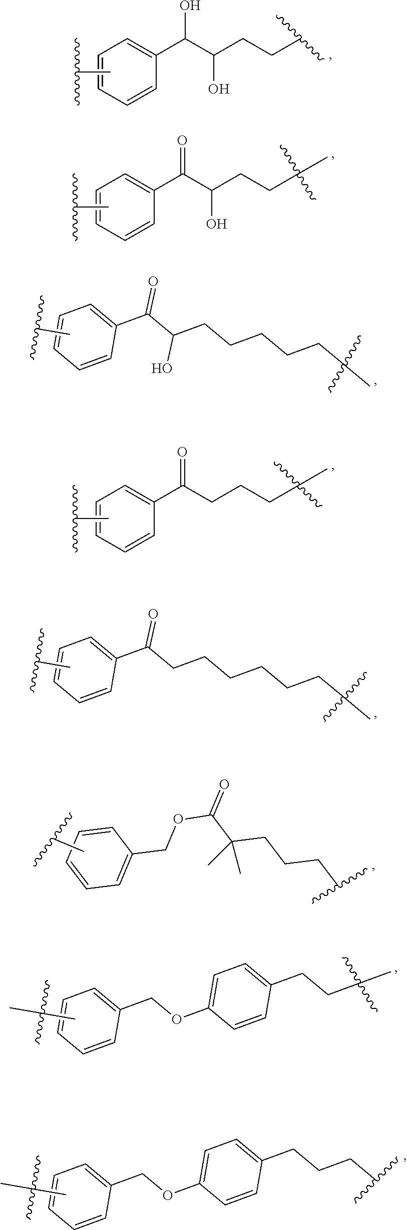

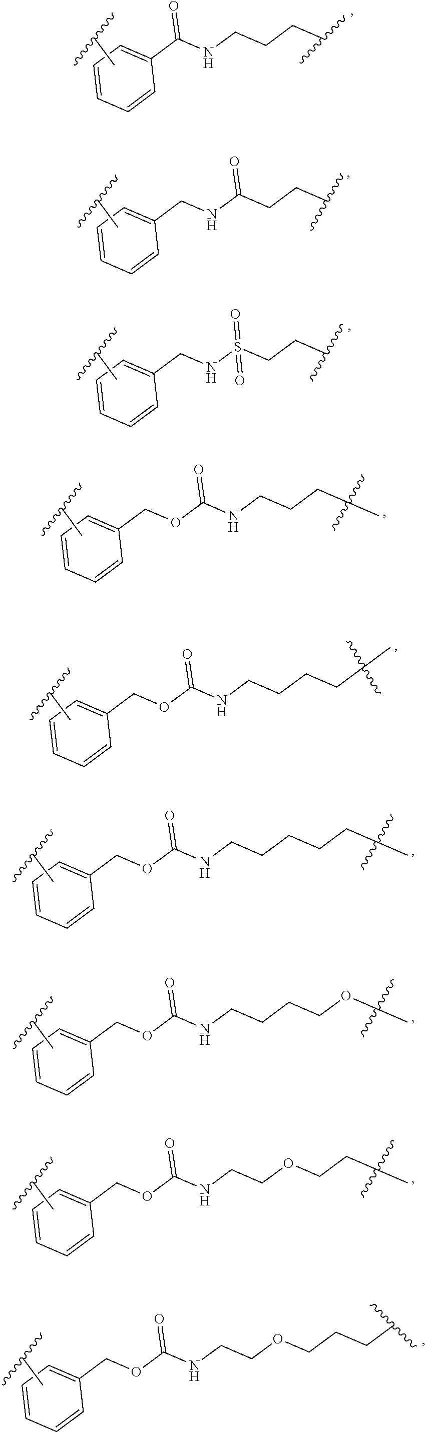

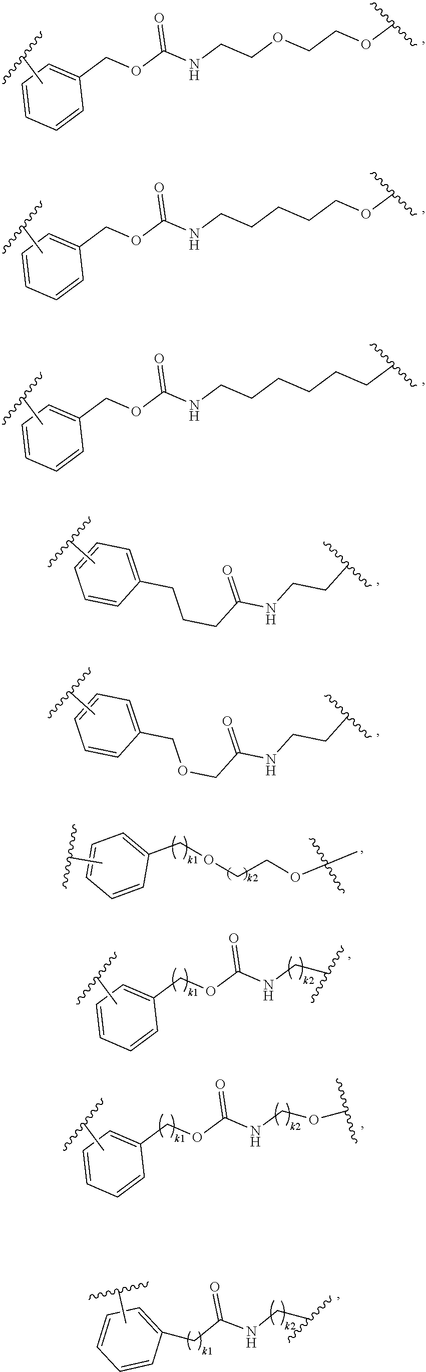

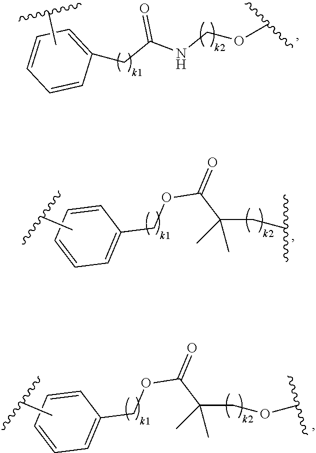

wherein each k1 and k2 is independently 0 or an integer selected from 1 to 10. In some embodiments, R* is attached to phenyl.

In some embodiments, a conjugate described herein comprises one or more independent radionuclides. In some embodiments, the conjugate comprises two radionuclides. In some embodiments, each of the one or more radionuclides is an alpha particle-emitting radionuclide. In some embodiments, each of the one or more radionuclides is a beta particle-emitting radionuclide.

In some embodiments, a radiolabeled conjugate described herein comprises an alpha particle-emitting radionuclide. In some embodiments, the alpha particle-emitting radionuclide is astatine-211 (²¹¹At).

In some embodiments, the conjugate comprises a covalently bound beta particle-emitting radionuclide. In some embodiments, the beta particle emitting radionuclide is iodine-131 (¹³¹I).

In some embodiments, the conjugate comprises a covalently bound β+positron-emitting radionuclide. In some embodiments, the β+ positron emitting radionuclide is fluorine-18 (¹⁸F).

In some embodiments, the conjugate comprises a gamma particle emitting radionuclide. In some embodiments, the gamma particle emitting radionuclide is iodine-123.

In some embodiments, provided herein are conjugates having the structures of the radiolabeled conjugates described herein, except that the covalent radionuclide is replaced with a surrogate (e.g., ¹³¹I replaced with iodine), i.e., a cold conjugates. In some embodiments, a covalent radionuclide of the radiolabeled conjugates described herein can be replaced with a surrogate (e.g., ¹³³I replaced with iodine) for testing and experimental purposes.

In some embodiments, radiopharmaceutical conjugates comprising covalently bound radionuclides described herein can be synthesized from a boronic acid, boronate, or stannane precursor. Boronic acid, boronate, and stannane precursors can be formed according to the following general reaction:

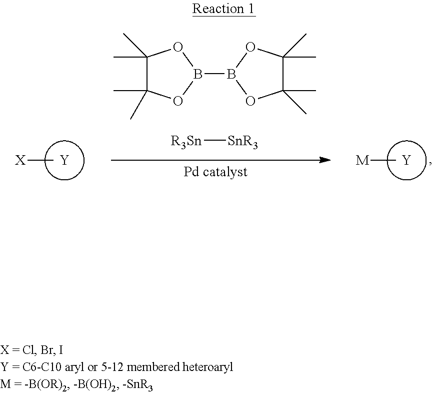

where R is a variable chemical moiety, for example alkyl or hydrogen. A halogen on ring Y, for example chloro, bromo, or iodo, can undergo a palladium mediated coupling reaction with a boronic acid, boronate, or stannane compound to form an intermediate compound wherein group M has replaced halogen X.

A covalent radionuclide, for example R*, such as fluorine-18 (¹⁸), iodine-131 (¹³¹I), iodine-123 (¹²³I), iodine-124 (¹²⁴I), iodine-125 (¹²⁵I) or astatine-211 (²¹¹At), or any other suitable radionuclide of Table 7 can be formed, for example, from the intermediate compound according to the following general reactions:

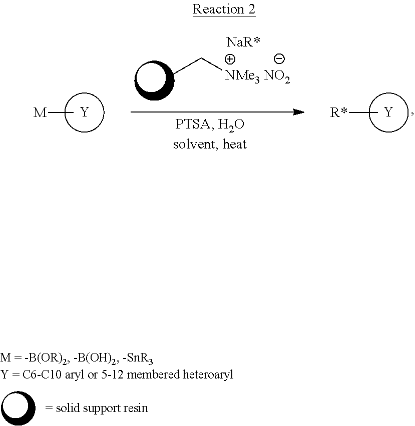

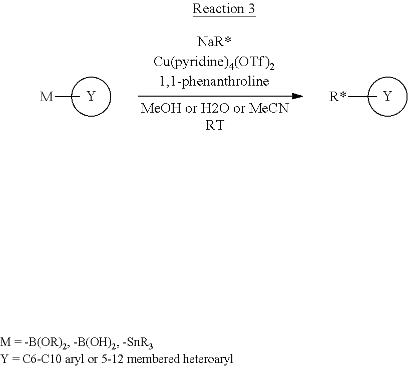

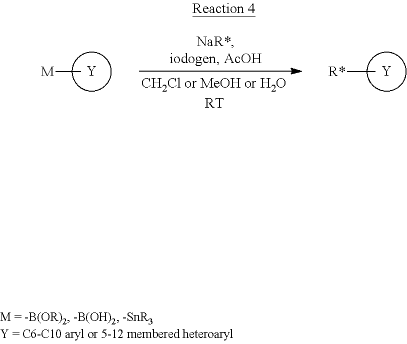

where R is a variable chemical moiety, for example alkyl or hydrogen.

In some embodiments, radiolabeled conjugates described herein can be synthesized from a chloro, bromo, or iodo precursor according to the following general reaction:

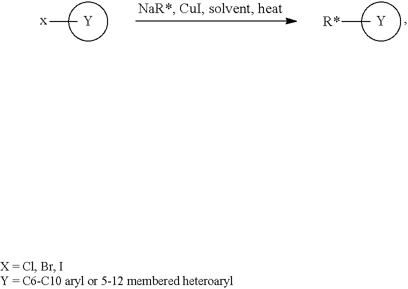

where R* is a radionuclide, for example, fluorine-18 (¹⁸F), iodine-131 (¹³¹I), iodine-123 (¹²³) iodine-124 (¹²⁴I), iodine-125 (¹²⁵I), astatine-211 (²¹¹At), or a radionuclide of Table 7 marked “Covalent”.

The radiolabeling reactions depicted above are used as example procedures in the synthesis of radiolabeled conjugates described herein. Additional reactions, including nucleophilic substitution, electrophilic substitution, isotopic exchanges, bromine-radioiodine exchange, radioiododestannylation, radioiododeboronation, and transition metal mediated halogen exchange are contemplated and procedures can be found in Berdal et al., “Investigation on the reactivity of nucleophilic radiohalogens with arylboronic acids in water; access to an efficient single-step method for the radioiodination and astatination of antibodies” Chemical Science 2021, 12, 1458, and Dubost et al., “Recent Advances in Synthetic Methods for Radioiodination” J. Org. Chem, 2020, 85, 13, 8300-8310, both of which are incorporated by reference herein in their entirety.

Accordingly, in one aspect, described herein is a method of synthesizing a radiopharmaceutical conjugate or salt or solvate or pharmaceutical composition thereof. In some embodiments, the method comprises replacing a halogen on a precursor conjugate with a radionuclide, e.g., a fluorine-18 (¹⁸F), iodine-131 (¹³¹I), iodine-123 (¹²³I), iodine-124 (¹²⁴I), iodine-125 (¹²⁵I), astatine-211 (²¹¹At), or a suitable radionuclide of Table 7. In some embodiments, the method comprises a transition metal (e.g., palladium) mediated coupling reaction with a boronic acid, boronate, or stannane compound to form an intermediate compound. In some embodiments, the method further comprises exchanging the boronic acid, boronate, or stannane group with the radionuclide, e.g., as illustrated above. In some embodiments, the method comprises nucleophilic substitution, electrophilic substitution, isotopic exchanges, bromine-radioiodine exchange, radioiododestannylation, radioiododeboronation, and/or transition metal mediated halogen exchange reactions.

In some embodiments, a radiopharmaceutical conjugate disclosed herein comprises a pseudo-radiometal, for example, an aluminum-¹⁸F complex. In some embodiments, the aluminum-¹⁸F complex is bound to a metal chelator.

### Labeled Compounds

In some embodiments, the conjugates described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled conjugates as pharmaceutical compositions. For example, the conjugates described herein may be artificially enriched in one or more particular isotopes. In some embodiments, the conjugates described herein may be artificially enriched in one or more isotopes that are not predominantly found in nature. In some embodiments, the conjugates described herein may be artificially enriched in one or more isotopes selected from deuterium (²H), tritium (²H), and/or carbon-14 (¹⁴C). All isotopic variations of the conjugates of the present disclosure are encompassed within the scope of the present disclosure. Examples of isotopes that can be incorporated into conjugates described herein, or a solvate, or stereoisomer thereof, include isotopes of hydrogen, carbon, nitrogen, oxygen, phosphorous, sulfur, fluorine, and chloride, such as ²H, ³H, ¹³C, ¹⁴C, ¹⁵N, ¹⁸O, ³¹P, ³²P, ³S, ¹⁸F, and ³⁰Cl, respectively. Conjugates described herein, and the pharmaceutically acceptable salts, solvates, or stereoisomers thereof which contain the aforementioned isotopes and/or other isotopes of other atoms are within the scope of this disclosure. Certain isotopically-labeled conjugates, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are notable for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled conjugate or a pharmaceutically acceptable salt, solvate, or stereoisomer thereof is prepared by any suitable method.

In some embodiments, the conjugates described herein are labeled by other means, including, but not limited to, the use of chromophores or fluorescent moieties, bioluminescent labels, or chemilumninescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions. As used herein, a “pharmaceutically acceptable salt” refers to any salt of a compound that is useful for therapeutic purposes of a subject.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or by separately reacting a purified compound in its free form with a suitable acid or base, and isolating the salt thus formed.

Examples of pharmaceutically acceptable salts include those salts prepared by reaction of the compounds described herein with a mineral acid, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate, metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid, metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxy ethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

In some embodiments, those compounds described herein which comprise a free acid group react with a suitable base, such as the hydroxide, carbonate, bicarbonate, or sulfate of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts, and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine, and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. This disclosure provides for methods of treating diseases by administering such solvates. This disclosure further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein. Accordingly, one aspect of the present disclosure pertains to hydrates and solvates of compounds of the present disclosure and/or their pharmaceutical acceptable salts, as described herein, that can be isolated and characterized by methods known in the art, such as, thennogravimetric analysis (TGA), TGA-mass spectroscopy, TGA-Infrared spectroscopy, powder X-ray diffraction (PXRD), Karl Fisher titration, high resolution X-ray diffraction, and the like.

### Preparation of the Compounds

The compounds used in the reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH, Inc. (Toronto, Canada), Bionet (Cornwall, U.K.), Chem Service Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire. UK), Frontier Scientific (Logan, UT), ICN Biomedicals. Inc. (Costa Mesa, CA), Key Organics (Cornwall, U.K.), Lancaster Synthesis (Windham, —NH). Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL). Riedel de Haen A G (Hanover. Germany). Spectrum Quality Product, Inc. (New Brunswick, NJ), TCI America (Portland, OR), Trans World Chemicals, Inc. (Rockville, MD), and Wako Chemicals USA, Inc. (Richmond. VA).

Suitable reference books and treatises that detail the synthesis of reactants useful in the preparation of compounds described herein, or provide references to articles that describe the preparation, include for example, “Synthetic Organic Chemistry”, John Wiley & Sons, Inc., New York: S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed, John Wiley & Sons, New York, 1992; J. March, “Advanced Organic Chemistry: Reactions, Mechanisms and Structure”, 4th Ed., Wiley-Interscience, New York, 1992. Additional suitable reference books and treatises that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March. J. “Advanced Organic Chemistry: Reactions. Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons. T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups”, John Wiley & Sons, in 73 volumes.

Specific and analogous reactants are optionally identified through the indices of known chemicals prepared by the Chemical Abstract Service of the American Chemical Society, which are available in most public and university libraries, as well as on-line. Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the compounds described herein is P. H. Stahl & C. G. Wermuth “Handbook of Pharmaceutical Salts”, Verlag Helvetica Chimica Acta, Zurich, 2002.

In one aspect, described herein is a method of making a conjugate that comprises a cyclic peptide, a metal chelator, optionally a linker, and optionally a radionuclide such as ¹⁷⁷Lu or 225Ac. In some embodiments, the conjugate is prepared by one or more of the following steps: (a) synthesizing the peptide sequence by solid phase peptide synthesis; (b) cyclizing the peptide by forming an intramolecular non-peptide bond; (c) coupling the metal chelator to the peptide; (d) and optionally labeling the conjugate with a radionuclide. In some embodiments, steps (a), (b), (c) and (d) are performed in the recited order. In some embodiments, synthesizing the peptide comprises synthesizing the peptide sequence in a protected form and performing a de-protecting reaction. In some embodiments, cyclizing the peptide comprises forming a non-peptide bond between the N-terminus and the C-terminus of the peptide. In some embodiments, cyclizing the peptide comprises forming a non-peptide bond between the N-terminus and a cysteine or homocysteine of the peptide. In some embodiments, cyclizing the peptide comprises forming a ring closing group selected from —C(═O)—CH₂—, —C(═O)—CH₂—S—, —S—, —CH═CH—, —NH—, -maleimide-S—, —C(═O)—CH₂—NH—, and —C(═O)—CH₂—O—. In some embodiments, cyclizing the peptide comprises forming a ring closing group of Table 4B. In some embodiments, cyclizing the peptide comprises reacting a pair of functional groups or amino acids described in Table 4C. In some embodiments, solid phase peptide synthesis can be replaced with other suitable peptide synthesis methods known in the art.

### Peptide Production

The peptide of the present technology may be produced by, for example, any known method for producing a peptide, such as the following:
a chemical synthesis method such as a liquid phase method, a solid phase method, a hybrid method combining a liquid phase method and a solid phase method, or the like;
a genetic recombination method; or the like.

In some of the instances where the peptide of the present technology is produced by a chemical synthesis method, it can be said that the peptide of the present technology is a synthetic peptide.

In the solid phase method, for example, a hydroxy group of a resin having a hydroxy group and a carboxy group of a first amino acid (normally a C-terminal amino acid of a target peptide) in which an α-amino group is protected by a protecting group are subjected to an esterification reaction. For the esterification catalyst, a known dehydrating and condensing agent such as 1-mesitylenesulfonyl-3-nitro-1, 2, 4-triazole (MSNT), dicyclohexylcarbodiimide (DCC), and diisopropylcarbodiimide (DIPCDI) may be used.

Next, the protecting group of the α-amino group of the first amino acid is removed, a second amino acid in which all functional groups except the carboxy group of the main chain are protected is added, and the carboxy group is activated, binding the first and second amino acids. Furthermore, the α-amino group of the second amino acid is deprotected, a third amino acid in which all functional groups except the carboxy group of the main chain are protected is added, the carboxy group is activated, binding the second and third amino acids. This is repeated, and after a peptide having a target length is synthesized, all of the functional groups are deprotected.

Examples of the resin for solid-phase synthesis include Merrifield resin. MBHA resin, Cl-Trt resin, SASRIN resin, Wang resin, Rink amide resin, HMFS resin, Amino-PEGA resin (Merck KGaA), HMPA-PEGA resin (Merck KGaA), and the like. These resins may be used after being washed using a solvent (dimethylformamide (DMF), 2-propanol, methylene chloride, and the like). A peptide chain can be cleaved from the resin by treating it with an acid such as TFA or hydrogen fluoride (HF).

Examples of the protecting group of the α-amino group include a benzyloxycarbonyl (Cbz or Z) group, tert-butoxycarbonyl (Boc) group, fluorenylmethoxycarbonyl (Fmoc) group, benzyl group, allyl group, allyloxycarbonyl (Alloc) group, and the like. The Cbz group may be deprotected by a treatment using hydrofluoric acid, hydrogenation, or the like, the Boc group may be deprotected by a treatment using trifluoroacetic acid (TFA), and the Fmoc group may be deprotected by a treatment using pipericine or pyrrolysine.

Examples, such as methyl ester, ethyl ester, allyl ester, benzyl ester, tert-butyl ester, cyclohexyl ester, and the like may be used to protect the α-carboxy group.

As other functional groups of an amino acid, the hydroxyl group of serine or threonine can be protected with a benzyl group or a tert-butyl group and the hydroxyl group of tyrosine can be protected with a 2-bromobenzyloxycarbonyl group or a tert-butyl group. The amino group of a lysine side chain or the carboxyl group of glutamic acid or aspartic acid can be protected in a manner similar to the α-amino group or α-carboxyl group.

Activation of the carboxy group may be performed using a condensing agent. Examples of the condensing agent include dicyclohexylcarbodiimide (DCC), diisopropylcarbodiimide (DIPCDI), 1-ethyl-3-(3-dimethylaminopropyl)carbodiimide (EDC or WSC), (1H-benzotriazole-1-yloxy)tris(dimethylamino)phosphonium hexafluorophosphate (BOP), 1-[bis(dimethylamino)methyl]-1H-benzotriazolium-3-oxide hexafluorophosphate (HBTU), and the like.

Cleavage of the peptide chain from the resin may be performed by treating the peptide chain using an acid such as TFA, hydrogen fluoride (HF), or the like.

Production of a peptide by a gene recombination method (translation/synthesis system) may be performed using a nucleic acid encoding the peptide. The nucleic acid encoding the peptide may be DNA or RNA.

The nucleic acid encoding the peptide may be prepared by a known method or a method equivalent thereto. For example, the peptide may be synthesized by an automated synthesizer. A restriction enzyme recognition site may be added to insert the obtained DNA into a vector. Alternatively, a base sequence encoding an amino acid sequence for splicing a formed peptide chain using an enzyme or the like may be incorporated. The peptide obtained may be converted from a free peptide to a salt thereof or from a salt thereof to a free peptide by a known method or a method based thereon.

As described above, when the peptide is fused to a cell-penetrating peptide or the like, the nucleic acid also includes a nucleic acid encoding the cell-penetrating peptide.

A chimeric protein expression method for expressing the target peptide as a chimeric peptide of another peptide may also be used to suppress degradation by a host-derived protease. In this case, a nucleic acid encoding the target peptide and the peptide bound thereto may be used as the nucleic acid.

Subsequently, an expression vector is prepared using the nucleic acid encoding the peptide. The nucleic acid may be digested as is or by a restriction enzyme, and alternatively, the nucleic acid may be inserted downstream of a promotor of the expression vector by adding a linker, or the like. Examples of the vector include an Escherichia coli-derived plasmid (pBR322, pBR325, pUC12, pUC13, pUC18, pUC19, pUC118, pBhiescript II, and the like), a Bacillus subtilis-derived plasmid (pUB1I10, pTP5, pCl912, pTP4, pE194, pC194, and the like), ayeast-derived plasmid (pSH19, pSH15, YEp, YRp, YIp, YAC, and the like), a bacteriophage (e phage, M13 phage, and the like), a virus (retrovirus, vaccinia virus, adenovirus, adeno-associated virus (AAV), cauliflower mosaic virus, tobacco mosaic virus, baculovirus, and the like), a cosmid, and the like.

The promoter may be selected appropriately according to the type of host. When the host is an animal cell, for example, a promoter derived from SV40 (simian virus 40) or a promoter derived from CMV (cytomegalovirus) may be used. When the host is Escherichia coli, a trp promoter, a T7 promoter, a lac promoter, or the like may be used.

The expression vector may incorporate, for example, a DNA replication starting point (ori), a selective marker (antibiotic resistance, auxotrophy, or the like), an enhancer, a splicing signal, a poly-A addition signal, a nucleic acid encoding a tag (FLAG, HA, GST, GFP, or the like), or the like.

Next, an appropriate host cell is transformed by the expression vector. The host may be appropriately selected in relation to the vector. Examples such as Escherichia coli, Bacillus subtilis (Bacillus), yeast, insects or insect cells, animal cells, or the like may be used as the host. As the animal cells, for example, HEK293T cells, CHO cells, COS cells, myeloma cells, HeLa cells, and Vero cells may be used. Transformation may be carried out according to a known method, such as a lipofection method, a calcium phosphate method, an electroporation method, a microinjection method, a gene gun method, or the like depending on the type of host. The target peptide is expressed by culturing a transformant according to a conventional method.

As for purification of the peptide from the transformant culture, cultured cells are recovered and then suspended in an appropriate buffer solution, followed by disruption of cells by a method such as sonication, freeze-thawing, or the like, and then a crude extract is obtained by centrifugation or filtration. When the peptide is secreted into the culture solution, a supernatant is recovered.

Purification of the crude extract or the culture supernatant may also be performed by a known method or a method equivalent thereto (for example, salting-out, dialysis, an ultrafiltration method, gel filtration method. SDS-PAGE method, ion exchange chromatography, affinity chromatography, reversed-phase high-performance liquid chromatography, and the like).

The obtained peptide may be converted from a free body to a salt or from a salt to a free body by a known method or a method equivalent thereto.

In one aspect, the translation/synthesis system may be a cell-free translation system. According to the cell-free translation system, a highly pure form of an expression product can generally be obtained without purification. The cell-free translation system includes, for example, a ribosome protein, an aminoacyl-tRNA synthase (ARS), a ribosome RNA, an amino acid, rRNA, GTP. ATP, a translation initiation factor (IF), an elongation factor (EF), a release factor (RF), and a ribosome regeneration factor (RRF), or another factor required for translation. An Escherichia coli extract or a wheat embryo extract may be added to increase expression efficiency. In addition, a rabbit red blood cell extract or an insect cell extract may be added.

By continuously supplying energy to a system including these using dialysis, a protein of several hundred μg to several mg/m may be produced in a non-limiting manner. The system may include an RNA polymerase to concurrently perform transcription of genomic DNA. Examples of commercially available cell-free translation systems that may be used include RTS-100 (registered trademark) by Roche Diagnostics K. K., PURE System by GeneFrontier Corporation, PUIRExpress In vitro Protein Synthesis Kit by New England Biolabs Inc., and the like for a system derived from Escherichia coli, and a system by ZOIGENE, CellFree Sciences Co., Ltd., or the like for a system using wheat embryo extract.

In the cell translation system, artificial aminoacyl-tRNA may be used and a desired amino acid or hydroxy acid may be linked (acylated) to a tRNA in place of an aminoacyl-tRNA synthesized by a natural aminoacyl-tRNA synthase. The aminoacyl-tRNA may be synthesized using an artificial ribozyme.

An example of the ribozyme includes a flexizyme (flexizyme) (H. Murakami, H. Saito, and H. Suga, (2003). Chemistry & Biology, Vol. 10, 655-662; and WO 2007/066627 and the like), all incorporated herein by reference. Flexizymes are also known under the names of prototype flexizyme (Fx), newly modified dinitrobenzyl flexizy me (dFx), enhanced flexizyme (eFx), aminoflexizyme (aFx), and the like.

A desired codon may be translated in association with the desired amino acid or hydroxy acid by using the tRINA produced by flexizyme and to which the desired amino acid or hydroxy acid is linked A specialty amino acid may be used as the desired amino acid. For example, an unnatural amino acid required for the above circularization may also be introduced into the binding peptide by this method.

Various methods commonly used in the technical field may be used for chemical synthesis of the peptide, including, for example, stepwise solid-phase synthesis, semisynthesis of peptide fragments undergoing conformationally supported religation, and chemical ligation. Synthesis of the peptide is chemical synthesis using various solid phase technologies described in, for example, K. J. Jensen. P. T. Shelton, S. L. Pedersen, Peptide Synthesis and Applications, 2nd Edition, Springer, 2013, and the like. A preferable strategy is based on a combination of an Fmoc group capable of temporarily protecting the α-amino group and being selectively removed using a base, and a protecting group that temporarily protects a side chain functional group and is stable under Fmoc deprotection conditions. Selection of this kind of general peptide side chain is known according to the aforementioned Peptide Synthesis and Applications, 2nd Edition; G. B. Fields, R. L. Noble, Solid Phase Peptide Synthesis Utilizing 9-Fluorenylmethoxycarbonyl Amino Acids, Int. J. Peptide Protein Res. 35, 1990, 161-214, and the like; however, preferable peptide side chain protecting groups include, for example, a benzyl group or a tert-butyl group and a trityl (Trt) group for the hydroxy group of serine or threonine: a 2-bromobenzyloxycarbonyl group or a tert-butyl group for the hydroxy group of tyrosine; a Boc group, a methyltetrazole thiol (Mtt) group, an Alloc group, and an ivDde group for the amino group of the lysine side chain; a Trt group or a Boc group for the imidazole group of histidine; a 2,2,4,6,7-pentamethyldihydrobenzofuran-S-sulfonyl group (Pbf) group for the guanidyl group of arginine; a tert-butyl group, an allyl group, and a 3-methylpentane (Mpe) group for carboxyl groups, such as glutamic acid and aspartic acid; a Trt group for the carboxamide group of glutamine or asparagine; or a Trt group and a monomethoxytrityl (Mmt) group for the thiol group of cysteine.

The peptide may be synthesized by a stepwise method on the solid-phase resin described above. The C-terminal amino acid to be used and all of the amino acids or peptides to be used for synthesis must be selectively removed during the process of synthesizing the α-amino protecting group. Preferably, the solid-phase resin described above is used, and once a C-terminal carboxyl group of a peptide having its N-terminal properly protected by Fmoc or the like or a C-terminal carboxyl group of an amino acid having its N-terminal protected by Fmoc is made into an activated ester by an appropriate reagent, this is then added to the amino group on the solid-phase resin to start. Subsequent elongation of the peptide chain may be achieved by removing the N-terminal protecting group (Fmoc group) then successively repeating condensation of the protected amino acid derivative according to the amino acid sequence of the target peptide. Note that these may release the target peptide in a final stage. Examples of releasing conditions are given in Teixeira. W. E. Benckhuijsen, P. E, de Koning, A. R. P. M. Valentijn, J. W. Drijfhout. Protein Pept. Lett., 2002, 9, 379-385, and the like, and the peptide may be released in a TFA solution containing water/silyl hydride/thiol as a scavenger in TFA. Typical examples include TFA/Water/TIS/DODT (volume ratio 925:2.5:2.5:2.5).

Synthesis of the peptide described in the present specification may be carried out using a single or multi-channel peptide synthesizer, for example, a Liberty Blue synthesizer from CEM Corporation, a Syro I synthesizer or a successor machine thereof from Biotage Japan, Ltd., or the like.

Activation of the carboxy group may be performed using a condensing agent. Examples of the condensing agent include dicyclohexylcarbodiimide (DCC), diisopropylcarbodiimide (DIPCDI), I-ethyl-3-(3-dimethylammnopropyl)carbodiimnide (EDC or WSC), (1H-benzotriazole-1-yloxy)tris(dimethylamino)phosphonium hexafluorophosphate (BOP), 1-[bis(dimethylamino)methyl]-1II-benzotriazolium-3-oxide hexafluorophosphate (HBTU), and the like.

Cyclization of the peptide may be carried out according to a known method. In a non-limiting manner, by designing the peptide to comprise two or more cysteine residues, for example, a cyclic structure may be formed by a disulfide bond after translation. Furthermore, according to the method of Goto et al. (Y. Goto, et al. ACS Chem. Biol. 3 120-129 (2008)), a peptide having a chloroacetyl group at its N-terminal may be synthesized by genetic code reprogramming technology and may also be circularized by disposing a cysteine residue containing a sulfur molecule in the peptide. Thus, a mercapto group spontaneously performs a nucleophilic attack on the chloroacetyl group after translation, and the peptide is circularized by thioether binding. Other amino acid combinations that bind to form a ring may be disposed within the peptide and circularized by genetic code reprogramming technology. Alternatively, circularization may be carried out by disposing an L-2-aminoadipic acid residue in the peptide and binding it to the main chain amino group of the N-terminal. In this manner, a known circularization method may be used without any particular limitation.

### Pharmaceutical Compositions

The radiopharmaceutical conjugate described herein, including e.g., pharmaceutically acceptable salt or solvate thereof can be administered per se as a pure chemical or as a component of a pharmaceutically acceptable formulation. In some embodiments, a conjugate described herein is combined with a pharnaceutically suitable or acceptable carrier selected on the basis of a chosen route of administration and standard pharmaceutical practice as described, for example, in Remington: The Science and Practice of Pharmacy (Geniaro, 21^{st} Ed. Mack Pub. Co., Easton, PA (2005)). Provided herein is a pharmaceutical composition comprising at least one conjugate described herein, or a stereoisomer, pharmaceutically acceptable salt, amide, ester, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, the disclosure provides a pharmaceutical composition comprising a herein described conjugate, or a pharmaceutically acceptable salt or solvate thereof, and a pharmaceutically acceptable excipient or carrier.

The conjugates and pharmaceutical compositions of the current disclosure can be administered by any suitable means, including oral, topical (including buccal and sublingual), rectal, vaginal, transdermal, parenteral, subcutaneous, intraperitoneal, intrapulmonary, intradermal, intrathecal and epidural and intranasal, etc.

### III. Method of Treatment

The present technology also relates to a method for treating a disease or disorder characterized by overexpression or decreased expression of GPC3 by administering the peptide of the present technology to a subject.

The present technology also relates to a use of the peptide of the present technology for the treatment of a disease or disorder characterized by overexpression or decreased expression of GPC3.

The present technology also relates to a use of the peptide for manufacturing a pharmaceutical composition for the treatment of a disease or disorder characterized by overexpression or decreased expression of GPC3.

The present technology also relates to the peptide of the present technology for use in a method for treating a disease or disorder characterized by overexpression or decreased expression of GPC3.

The present technology also relates to a kit for use in a method of diagnosing disease or disorder characterized by overexpression or decreased expression of GPC3 by determination of the expression level of GPC3.

The present technology also relates to a composition for use in a method of diagnosing disease or disorder characterized by overexpression or decreased expression of GPC3.

The present technology also relates to use of a peptide or a salt thereof for use in a method of diagnosing disease or disorder characterized by overexpression or decreased expression of GPC3.

The present technology also provides methods of treating a disease or condition in a subject in need thereof. In some embodiments, the disease or disorder is characterized by overexpression or decreased expression of GPC3 in diseased tissue. In some embodiments, the disease or disorder is characterized by overexpression of GPC3 in diseased tissue. In some embodiments, the disease or disorder is characterized by decreased expression of GPC3 in diseased tissue. In some embodiments, the methods comprise administering a peptide described herein, a conjugate described herein, or a pharmaceutically acceptable salt or solvate thereof described herein, or a pharmaceutical composition comprising the same, to the subject in need thereof. In some embodiments, provided herein is a method of providing a therapeutic and/or prophylactic benefit to a subject in need thereof comprising administering a peptide, a conjugate, or a pharmaceutical composition described herein.

In some embodiments, the methods comprise administering to a subject a therapeutically effective amount of a peptide, a conjugate, or a pharmaceutically acceptable salt or solvate thereof. In some embodiments, the peptide, conjugate or pharmaceutically acceptable salt or solvate thereof is administered in a pharmaceutical composition. In some embodiments, the subject has cancer. In some embodiments, the cancer is a solid tumor or hematological cancer.

In one aspect, provided herein is a method of treating cancer by administering a herein described peptide, conjugate or a pharmaceutically acceptable salt or solvate thereof to a subject in need thereof. According to a further aspect of the disclosure, there is provided a peptide, or a drug conjugate thereof as defined herein, for use in preventing, suppressing or treating a disease or disorder characterized by overexpression or decreased expression of GPC3 in diseased tissue. In some embodiments, the disease or disorder is characterized by overexpression of GPC3 in diseased tissue (such as a tumor). In one embodiment, the GPC3 is mammalian GPC3. In a further embodiment, the mammalian GPC3 is human GPC3.

In one aspect, provided herein is a method of preventing, suppressing or treating a disease or disorder characterized by overexpression or decreased expression of GPC3 in diseased tissue. In some embodiments, the disease or disorder is characterized by overexpression of GPC3 in diseased tissue (such as a tumor), which comprises administering to a patient in need thereof a peptide or a conjugate described herein. In some embodiments, the disease or disorder characterized by overexpression of GPC3 in diseased tissue is a cancer.

Non-limiting examples of cancers to be treated by the methods of the present disclosure can include hematological cancer or solid tumor, such as melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In some embodiments, a subject or population of subjects to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a subject or population of subjects to be treated with a pharmaceutical composition of the present disclosure have a hematological cancer. In some embodiments, the subject has a hematological cancer such as Diffuse large B cell lymphoma (“DLBCL”), Hodgkin's lymphoma (“IL”), Non-Hodgkin's lymphoma (“NHL”). Follicular lymphoma (“FL”), acute myeloid leukemia (“AML”), or Multiple myeloma (“MM”). In some embodiments, a subject or population of subjects to be treated having the cancer selected from the group consisting of ovarian cancer, lung cancer, and melanoma. In some embodiments, the cancer is selected from hepatocellular carcinoma, squamous cell carcinoma of the lung, lung adenocarcinoma, germ cell tumors, hepatoblastoma, wilms tumor, malignant rhabdoid tumors, rhabdomyosarcoma, liposarcoma, thyroid cancers, pancreatic cancer, small bowel cancer, small cell neuroendocrine carcinoma (SCNC), castration resistant prostatic adenocarcinoma, ovarian cancer, gastric cancer, esophageal carcinoma and malignant melanoma. In some embodiments, the castration resistant prostatic adenocarcinoma is hormonally treated. In some embodiments, the cancer iCs neuroendocrine prostate cancer (NEPC).

In some embodiments, provided herein are methods and compositions for treating a disease or condition. Exemplary disease or condition includes refractory or recurrent malignancies whose growth may be inhibited using the methods of treatment of the present disclosure. In some embodiments, the disease or condition is a cancer. In some embodiments, the cancer is breast cancer, head and neck squamous cell carcinoma, non-small cell lung cancer, hepatocellular cancer, bladder cancer, colorectal cancer, gastric adenocarcinoma, ovarian cancer, melanoma, or advanced cancer. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, the cancer is neuroendocrine prostate cancer (NEPC). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is pancreatic cancer. In some embodiments, a cancer to be treated by the methods of the present disclosure is non-small cell lung cancer, ovarian cancer, or bladder cancer. In some embodiments, a cancer to be treated by the methods of the present disclosure is non-small cell lung cancer. In some embodiments, a cancer to be treated by the methods of the present disclosure is bladder cancer. In some embodiments, a cancer to be treated by the methods of the present disclosure is ovarian cancer. In some embodiments, a cancer to be treated by the methods of the present disclosure is hepatocellular carcinoma, squamous cell carcinoma of the lung, lung adenocarcinoma, germ cell tumors, hepatoblastoma, wilms tumor, malignant rhabdoid tumors, rhabdomyosarcoma, liposarcoma, thyroid cancers, pancreatic cancer, small bowel cancer, small cell neuroendocrine carcinoma (SCNC), hormonally treated, castration resistant prostatic adenocarcinoma, ovarian cancer, gastric cancer, esophageal carcinoma, or malignant melanoma.

Further examples of cancers (and their benign counterparts) which may be treated include, but are not limited to tumors of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); hematological malignancies (i.e. leukemias, lymphomas) and premalignant hematological disorders and disorders of borderline malignancy including hematological malignancies and related conditions of lymphoid lineage (for example acute lymphocytic leukemia (ALL), chronic lymphocytic leukemia, B-cell lymphomas such as diffuse large B-cell lymphoma, follicular lymphoma, Burkitt's lymphoma, mantle cell lymphoma, T-cell lymphomas and leukemias, natural killer cell lymphomas, Hodgkin's lymphomas, hairy cell leukemia, monoclonal gammopathy of uncertain significance, plasmacytoma, multiple myeloma, and post-transplant lymphoproliferative disorders), and hematological malignancies and related conditions of myeloid lineage (for example acute myelogenous leukemia, chronic myelogenous leukemia, chronic myelomonocytic leukemia, hypereosinophilic syndrome, myeloproliferative disorders such as polycythemia vera, essential thrombocythemia and primary myelofibrosis, myeloproliferative syndrome, myelodysplastic syndrome, and promyelocytic leukemia); tumors of mesenchymal origin, for example sarcomas of soft tissue, bone or cartilage such as osteosarcomas, fibrosarcoma's, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumors, benign and malignant histiocytomas, and dermatofibrosarcoma protuberans; tumors of the central or peripheral nervous system (for example astrocytoma's, gliomas and glioblastomas, meningiomas, ependymomas, pineal tumors and schwannomas); endocrine tumors (for example pituitary tumors, adrenal tumors, islet cell tumors, parathyroid tumors, carcinoid tumors and medullary carcinoma of the thyroid); ocular and adnexal tumors (for example retinoblastoma); germ cell and trophoblastic tumors (for example teratomas, seminomas, dysgerminomas, hydatidiform moles and choriocarcinomas); and pediatric and embryonal tumors (for example medulloblastoma, neuroblastoma, Wilms tumor, and primitive neuroectodermal tumors); or syndromes, congenital or otherwise, which leave the patient susceptible to malignancy (for example Xeroderma Pigmentosum).

In some embodiments, the cancer is selected from glioblastoma, prostate cancer, lung cancer, breast cancer, gastric cancer, ovarian cancer, gladder cancer, colon cancer, esophageal cancer, multiple myeloma and fibrosarcoma. In some embodiments, the cancer is selected from: breast cancer, hung cancer, gastric cancer, pancreatic cancer, prostate cancer, liver cancer, glioblastoma and angiogenesis. In some embodiments, the cancer is selected from: prostate cancer, lung cancer (such as non-small cell lung carcinomas (NSCLC)), breast cancer (such as triple negative breast cancer), gastric cancer, ovarian cancer, esophageal cancer, multiple myeloma and fibrosarcoma. In some embodiments, the cancer is prostate cancer. In some embodiments, the conjugate is useful for preventing, suppressing or treating solid tumors such as fibrosarcoma's and breast, and non-small cell lung carcinomas. In some embodiments, the cancer is selected from lung cancer, such as non-small cell lung carcinomas (NSCLC). In some embodiments, the cancer is breast cancer. In some embodiments, the breast cancer is triple negative breast cancer. In some embodiments, the breast cancer is Herceptin resistant breast cancer. In some embodiments, the subject has failed to respond to Herceptin. In some embodiments, the cancer is gastric cancer. In some embodiments, the cancer is ovarian cancer. In some embodiments, the cancer is esophageal cancer. In some embodiments, the cancer is multiple myeloma. In some embodiments, the cancer is fibrosarcoma.

In some embodiments, provided herein are methods for killing a cell comprising contacting the cell with a peptide, a conjugate, or a pharmaceutically acceptable salt or solvate thereof. In some embodiments, the cell expresses GPC3. In some embodiments, the cell over-expresses GPC3. In some embodiments, the conjugate or pharmaceutically acceptable salt or solvate thereof binds to a structure on the cell, wherein the structure is an GPC3.

In some embodiments, the conjugate or pharmaceutically acceptable salt or solvate thereof releases a number of alpha particles by natural radioactive decay. In some embodiments, the conjugate or pharmaceutically acceptable salt or solvate thereof releases a number of beta particles, gamma rays, and/or Auger electrons by natural radioactive decay. The conjugate described herein can kill a cell by radiation. In some embodiments, the conjugate kills the cell directly by radiation. In some embodiments, the radiation creates, in the cell, oxidized bases, abasic sites, single-stranded breaks, double-stranded breaks, DNA crosslink, chromosomal rearrangement, or a combination thereof. In some embodiments, the conjugate kills the cell by inducing double-stranded DNA breaks. In some embodiments, the released alpha particles are sufficient to kill the cell. In some embodiments, the released alpha particles are sufficient to stop cell growth. In some embodiments, the conjugate kills the cell indirectly via the production of reactive oxygen species (ROS) such as free hydroxyl radicals. In some embodiments, the cell is a cancer cell. In some embodiments, the method comprises killing a cell with an alpha-particle emitting radionuclide.

After contacting a cell, the described peptide or conjugate can be internalized by the cell. The internalization can be mediated by cell receptors, cell membrane endocytosis, etc. In some embodiments, the described peptide or conjugate is internalized by a cell through GPC3. In some embodiments, rapid internalization rate into cancer cells accompanied by a slow externalization rate can offer therapeutic benefit.

In one aspect, the disclosed peptide, conjugate or a pharmaceutically acceptable salt or solvate thereof is configured to treat cancer by ablating tumor cells. In some embodiments, the peptide, conjugate or a pharmaceutically acceptable salt or solvate thereof does not modulate the biology of the tumor cell and/or the surrounding stroma. In some embodiments, the conjugate or a pharmaceutically acceptable salt or solvate thereof does not modulate immune cells. In some embodiments, the ablating of tumor cells can lead to a downstream immunological cascade.

In addition to the methods of treatment described above, the radiopharmaceutical conjugates and compositions described herein can be used to image, and/or as part of a treatment for diseases. Conjugates for imaging applications, e.g., single-photon emission computed tomography (SPECT) and positron emission tomography (PET), can comprise a radionuclide suitable for use as imaging isotopes (e.g., Dx in Table 7) such as the isotopes in Table 7. Accordingly, the radiopharmaceutical conjugate can be administered as a companion diagnostic.

In one aspect, described herein is a method of treatments that comprises administering a first conjugate and a second conjugate. The first conjugate can be used as companion diagnostics and the second conjugate can be used for therapeutics. In some embodiments, the first conjugate and the second conjugate have the same structure except for the radionuclide. In some embodiments, the first conjugate comprises a gamma particle emitting radionuclide. In some embodiments, the first conjugate comprises a diagnostic radionuclide of Table 7 labeled “Dx.” In some embodiments, the first conjugate comprises a radionuclide selected from Lu-177, In-111, Ga-68, Cu-64, and Zr-89. In some embodiments, the second conjugate comprises an alpha or beta-particle emitting radionuclide. In some embodiments, the second conjugate comprises a therapeutic radionuclide of Table 7 labeled “Tx”. In some embodiments, the second conjugate comprises Ac-225. In some embodiments, the method comprises administering (i) a first conjugate comprising a radionuclide configured for companion diagnostic (such as PET imaging) and (ii) a second conjugate comprising a radionuclide selected from an alpha or beta-particle emitter, wherein the first and the second conjugate have the same structure except for the radionuclide.

In one aspect, described herein is a method of diagnosing or imaging a cancer in a subject in need thereof, comprising administering to the subject a peptide, a conjugate or a pharmaceutical composition described herein.

In some embodiments, the subject is 1 to 100 years old. In some embodiments, the subject is 5 to 10, 5 to 15, 5 to 18, 5 to 25, 5 to 35, 5 to 45, 5 to 55, 5 to 65, 5 to 75, 10 to 15, 10 to 18, 10 to 25, 10 to 35, 10 to 45, 10 to 55, 10 to 65.10 to 75.15 to 18, 15 to 25, 15 to 35, 15 to 45, 15 to 55, 15 to 65, 15 to 75, 18 to 25, 18 to 35, 18 to 45, 18 to 55, 18 to 65, 18 to 75.25 to 35, 25 to 45, 25 to 55, 25 to 65, 25 to 75, 35 to 45, 35 to 55, 35 to 65, 35 to 75, 45 to 55, 45 to 65, 45 to 75, 55 to 65, 55 to 75, or 65 to 75 years old. In some embodiments, the subject is at least 5, 10, 15, 18, 25, 35, 45, 55, or 65 years old. In some embodiments, the subject is at most 10, 15, 18, 25, 35, 45, 55, 65, or 75 years old.

The amount of radiopharmaceutical conjugates described herein or a pharmaceutically acceptable salt or stereoisomer thereof and/or pharmaceutical compositions administered can be sufficient to deliver a therapeutically effective dose of the particular subject. In some embodiments, radiopharmaceutical conjugate dosages can be between about 0.1 μg and about 50 mg per kilogram of body weight. In some embodiments, the dose of the radiopharmaceutical conjugate or a pharmaceutically acceptable salt or stereoisomer thereof described herein for the described methods is about 0.001 mg to about 1000 mug per dose for the subject being treated.

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### A: Synthesis of the Conjugates

Unless otherwise stated in the present specification, the following abbreviations are used according to the following meanings:
Alloc allyloxycarbonyl
aq. aqueous
Biotin-OSu biotin N-hydroxysuccinimide ester (CAS 35013-72-0)
Boc tert-butyloxycarbonyl
CIAcOH chloroacetic acid
CIAcOSu A-succinimidyl 2-chloroacetate (CAS 27243-15-8)
DCM dichloromethane (CAS 75-09-2)
DIC N,N′-diisopropylcarbodiimide (CAS 693-13-0)
DIPEA, DIEA N,N-diisopropylethylamine (CAS 7087-68-5)
DMEM Dulbecco's Modified Eagle Medium
DMF N,N-dimethylformamide (CAS 68-12-2)
DODT 2,2′-(ethylenedioxy)diethanethiol (CAS 14970-87-7)
EDCI-HCl N-(3-dimethylaminopropyl)-N-ethylcarbodiimide hydrochloride (CAS 25952-53-8)
EDTA ethylenediaminetetraacetic acid
EMEM Eagle's minimal essential medium
eq equivalent
Et ethyl
Et₃N, TEA triethylamine (CAS 121-44-8)
FBS fetal bovine serum
Fmoc 9-fluorenylmethoxycarbonyl
hr hour
HATU 1-[bis(dimethylamino)methylene]-111-1,2,3-triazolo[4,5-b]pyridinium-3-oxide hexafluorophosphate (CAS 148893-10-1)
HOSu N-hydroxysuccinimide (CAS 6066-82-6)
iPrOH, IPA isopropanol
M molar
min minutes
NHS N-hydroxysuccinimide (CAS 6066-82-6)
NMP N-methylpyrrolidone (CAS 872-50-4)
Pd(PPh₃)A tetrakis(triphenylphosphine)palladium(0) (CAS 14221-01-3)
PG protecting group
Ph phenyl
RP reverse phase
rpm rotations per minute
rt room temperature
SAPE Streptavidin, R-PhycoerTthrin conjugate
SPPS solid phase peptide synthesis
Su succinimidyl
SulfoCy5-sulfo Cyanine5 (CAS 2791287-13-1)
SulfoCv5-OSu, (CAS 146368-14-1)
SulfoCy5-NHS see SulfoCy5-OSu
tert tertiary
TFA trifluoroacetic acid (CAS 76-05-1)
TIS triisopropylsilane (CAS 6485-79-6)
tr retention time
Trt trityl
Py AOP (7-azabenzotriazol-1-yloxy)trispyrrolidinophosphoniumn hexafluorophosphate (CAS 156311-83-0)
MeCN acetonitrile
AcOH acetic acid
Et₂O diethyl ether
AA amino acid
Me methyl
HFIP 1,1,1,3,3,3-hexafluoro-2-propanol (CAS 920-66-1)
PhSiH3 phenylsilane (CAS 694-53-1)
tBu tertiary butyl
DMSO dimethylsulfoxide
Mpe 3-Methyl-pent-3-yl

### Example A1. Analytical Conditions

Solid phase peptide synthesis (SPPS) was performed in a standard manual reaction vessel under nitrogen. Sieber Resin was purchased from Sunresin New Materials Co. (China). Fmoc protected amino acids were purchased from GL Biochem (China). HBTU and HATU were purchased from Highfine Biotech Co. (China). Piperidine was purchased from Damao Chemical Reagent Factory (China). The peptides and their derivatives were purified on a Gilson GX-281 preparative HPLC system using reverse-phase C18 columns (Gemini, 5 μm, 110 Å+ luma, 10 μm, 100 Å) at30° C. HPLC solvents consisted of H₂O containing 0.075% trifluoroacetic acid (mobile phase A) and acetonitrile (mobile phase B).

High performance liquid chromatography (1HPLC) analyses were performed on an Agilent 1260 series equipped with a binary pump G7112A, micro vacuum degasser, standard autosampler ALS G7129A, thermostatted column compartment TCC G7116A, variable wavelength detector VrWD G7114A, and data were analyzed by OpenLab CDS 2.2 network workstation software from Agilent Technologies. HPLC solvents consisted of H₂O containing 0.1% trifluoroacetic acid (mobile phase A) and acetonitrile containing 0.075% trifluoroacetic acid (mobile phase B). Conditions: a Phenomenex Gemini-NX C-18 (5 μm, 110 Å, 4.6×250 mm) column was used with a flow rate of 1.0 ml/min.

LC-MS analyses were carried out on an Agilent 1200 series coupled to an Agilent MSD G6125C. equipped with a binary pump G7112 Å, micro vacuum degasser, standard autosampler ALS G7129 Å, thermostatted column compartment TCC G7116 Å, variable wavelength detector VWD 07114 Å, and data were analyzed by OpenLab CDS 2.3 standalone workstation software from Agilent Technologies. HPLC solvents consisted of H₂O containing 0.1% trifluoroacetic acid (mobile phase A) and acetonitrile containing 0.075% trifluoroacetic acid (mobile phase B). Conditions: a Waters Xbridge C-18 (3.5 μm, 3.1×30 mm) column was used with a flow rate of 1.2 mL/min.

### Example A2: Synthesis of Lu-C-72

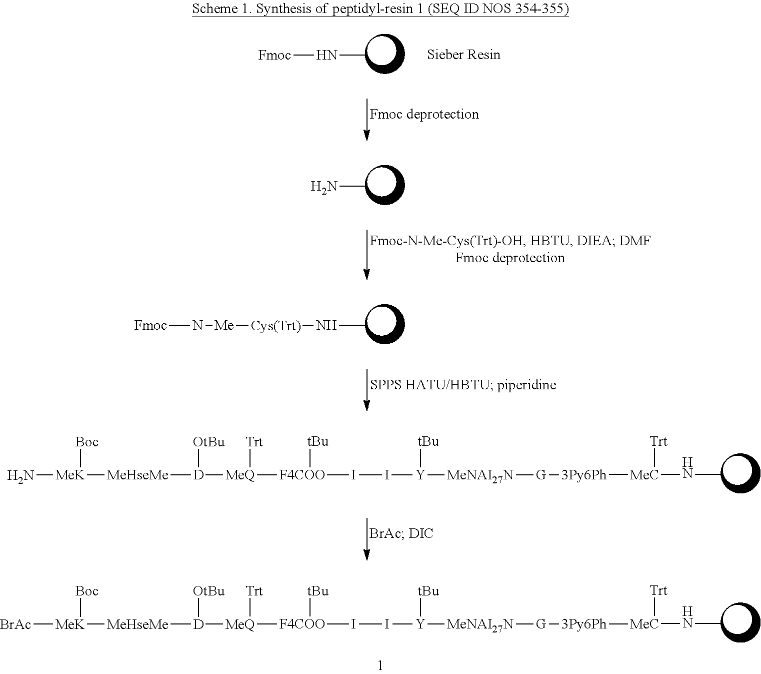

To the swollen Sieber Resin (0.3 mmol, 0.44 mmol/g, 1.00 equiv) was removed via 20 min agitation with 20% piperidine in DMF followed by filtration and washing. Then the resin was added Fmoc-N-Me-Cys(Trt)-OH (0.54 g, 0.9 mmol) HBTU (0.32 g, 0.85 mmol) and DIEA (0.23 g, 1.8 mmol) in dry DMF. The mixture was agitated for 30 min under nitrogen. After the reaction solution was removed through filtration, the resin was washed three times with DMF (10 mL). The Fmoc protecting group was removed via 30 min agitation with 20% piperidine in DMF followed by filtration and washing.

Subsequent amino acids were coupled using Fmoc-protected amino acid (3.00 equiv), HATU/HBTU (2.85 equiv) and DIEA (6.00 equiv) in dry DMF, shaking for 30 min. Pre-activation of any amino acid was not performed prior to coupling. Between amino acid couplings, the Fmoc protecting group was removed via 30 min agitation with 20% piperidine in DMF followed by filtration and washing. Success of Fmoc removal steps and amino acid couplings were monitored qualitatively using a ninhydrin test.

At the last step, to the swollen Sieber resin were added BrAc (0.25 g, 1.80 mmol), and DIC (0.23 g, 1.80 mmol) in DMF (5 mL). The mixture was agitated for 10 min under nitrogen. After the reaction solution was removed through filtration, the resin was washed three times with DMF (8 mL).

Synthesis of the cyclic peptide and La complex (SEQ ID NOS 356-359 and 258, respectively, in order of appearance).

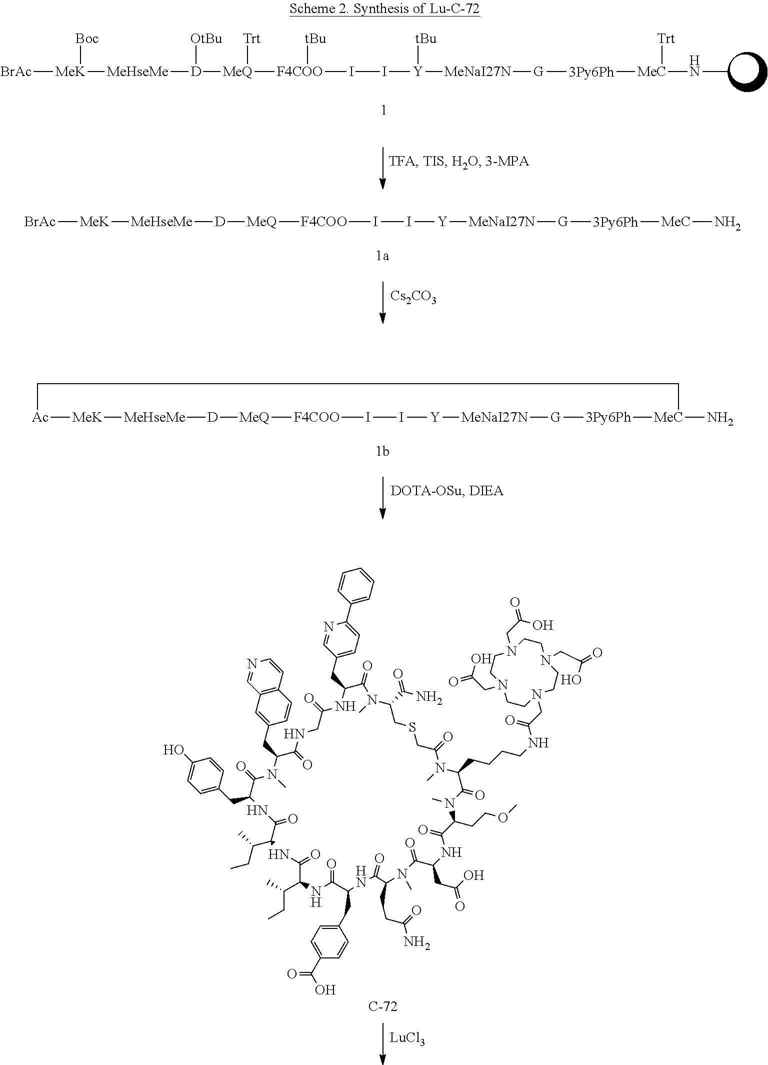

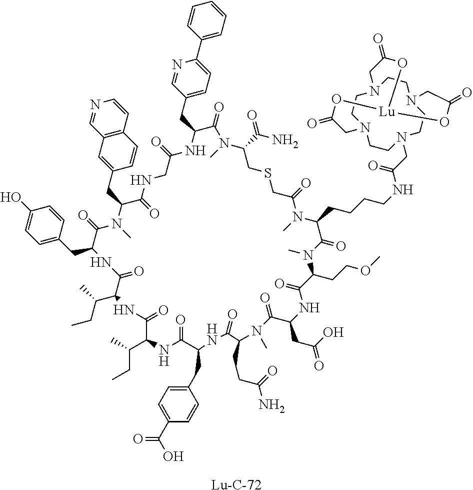

After the resin was washed three times with MeOH and dried under vacuum, a cocktail of trifluoroacetic acid/H₂O/triisopropylsilane/3-mercaptopropionic acid (90:2.5:2.5:5.0) was added. The resulting mixture was stirred for 1.5 h at room temperature. Cold isopropyl ether was added. The precipitated crude linear peptide-1a was collected through filtration and dried under vacuum.

cyclization of peptide-1a: To a solution of crude 1a (480 mg) in water (120 mL) and MeCN (60 ml) were added Cs₂CO₃ (3.0 eq). The resulting mixture was stirred at room temperature for 0.5 hour. The pH of the solution was then adjusted to 6-7 using 1.0 N HCl. After lyophilization, the crude was purified by preparative HPLC to afford peptide-1b (123 mg, 85% purity) as a white solid.

To a solution of peptide-1b (123 mg) in DMF (6 mL) were added DOTA-OSu (1.5 eq), DIEA (3 eq). The resulting mixture was heated to 40° C. and stirred at 40° C. 1 hour, the crude was purified by preparative HPLC to afford C-72 (65 mg, 94.21% purity).

Lu³⁺ complexation: To a solution of C-72 (40 mg, 94.21% purity) in H₂O (2 mL) and MeCN (1 ml) was added LuC₃ (5 eq) and then IM Na₂CO₃ aqueous solution was adjusted to pH=5-6. The resulting mixture was stirred at 40° C. for 1 h. After filtration, the crude product was purified by preparative HPLC to afford Lu-C-72 (22.4 mg, 94.43% purity).

### Example A3: Synthesis of Lu-C-45

### Synthesis of peptidyl-resin 2

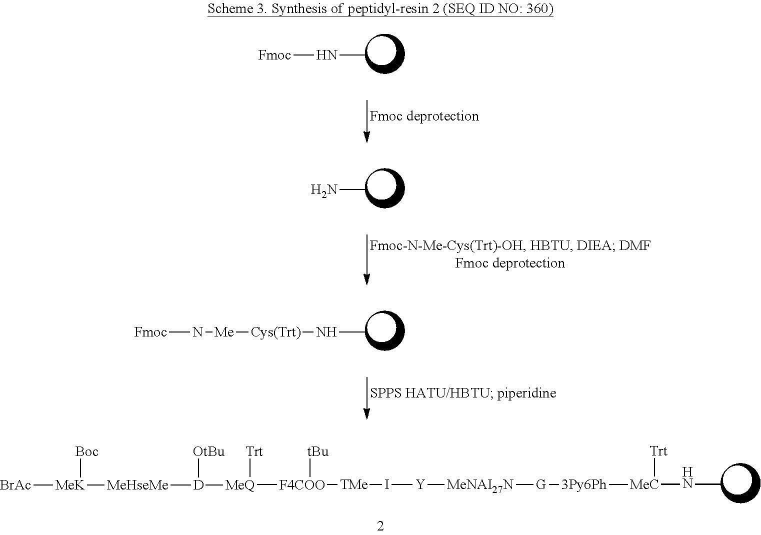

Similarly to Example A2 Lu-C-72, the linear polypeptide was synthesized on Fmoc-Sieber amide resin (0.25 mmol) using Fmoc-AAs listed in the above scheme and below Table 8.

TABLE 8

| order of Fmoc-AA | AA position in | |
|---|---|---|
| used in the SPPS | the cyclic peptide | Fmoc-AA |
| 1 | X12 | Fmoc-MeC(Trt)-OH |
| 2 | X11 | Fmoc-3Py6Ph-OH |
| 3 | X10 | Fmoc-G-OH |
| 4 | X9 | Fmoc-MeNal27N |
| 5 | X8 | Fmoc-Y(tBu)-OH |
| 6 | X7 | Fmoc-I-OH |
| 7 | X6 | Fmoc-TMe-OH |
| 8 | X5 | Fmoc-F4COO(tBu)-OH |
| 9 | X4 | Fmoc-Me(Trt)-OH |
| 10 | X3 | Fmoc-D(Mpe)-OH |
| 11 | X2 | Fmoc-MeHseMe-OH |
| 12 | X1 | Fmoc-MeK(Boc)-OH |

### Synthesis of Lu-C-45 (SEQ ID NOS 361-364 and 253, Respectively, in Order of Appearance)

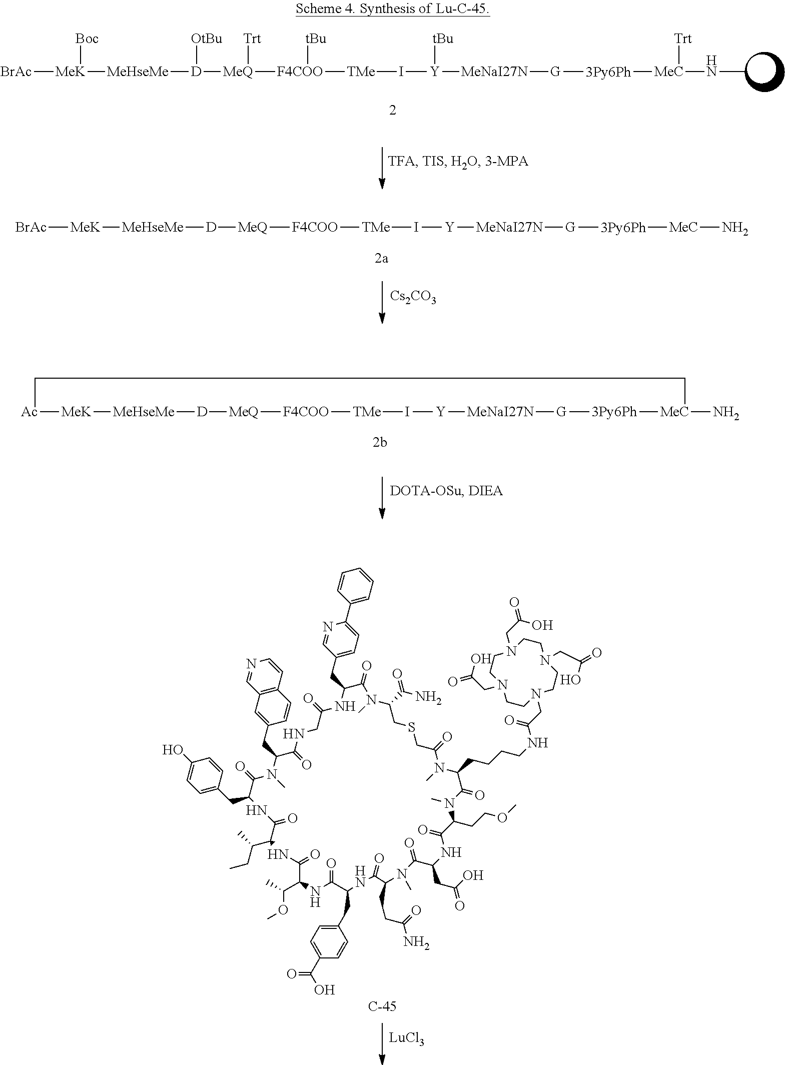

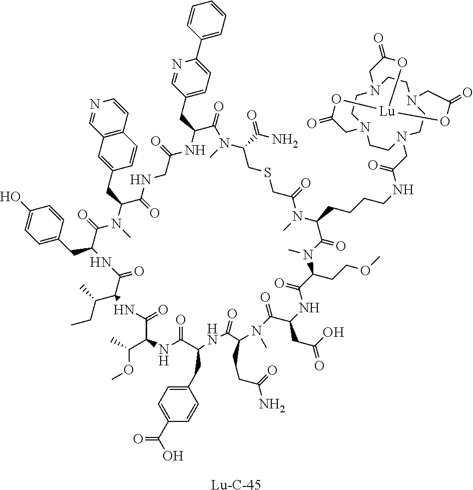

Similarly to Example A2 Lu-C-72, the crude containing the linear peptide (0.25 mmol as theoretical based on the resin used) was dissolved in CH3CN/H₂O (100 mL), and then added Et3N (2.5 mmol, 348 μL), and then shaken at room temperature for 18 h. This mixture was lyophilized to give the crude powder, then purified by preparative HPLC. Pure fractions were combined and lyophilized to afford the title macrocyclic peptide 2b. ESI-MS m/z: 890.7 [M+2H]2+.

Coupling DOTA-NHS and Lu³⁺ complexation: to a solution of the macrocyclic peptide 2b (30 mg, 0.014 mmol) in DMF (0.7 mL) at 0° C. were added 0.12 M DOTA-OSu (0.085 mmol) in water (0.7 mL), and DIEA (0.127 mmol, 22.2 μL), and then the mixture was stirred at 22° C. for 1 hour. The reaction was then quenched with DIEA (0.127 mmol, 22.2 μL). The mixture was then stirred at 22° C. for 1 hour. To the mixture was added 0.05 M LuCl₃ (0.156 mmol) in 50 mM NH₄OAc buffer (3.1 mL). The mixture was stirred at 0° C. for 1 hour, and then directly by preparative HPLC to afford the title nacrocyclic peptide Lu-C-45, ESI-MS n/z: 780.0 [M+3H]3+.

### Example A4: Synthesis of Conjugates

Compounds in Table 9 were prepared analogously to the above Examples A2-A3.

TABLE 9

| | Conjugate | SEQ | Exact | |
|---|---|---|---|---|
| | ID | ID | mass | Observed |
| | (SEQ ID | of the | (g/ | mass |
| Structure | NO:) | peptide | mol) | (g/mol) |
| 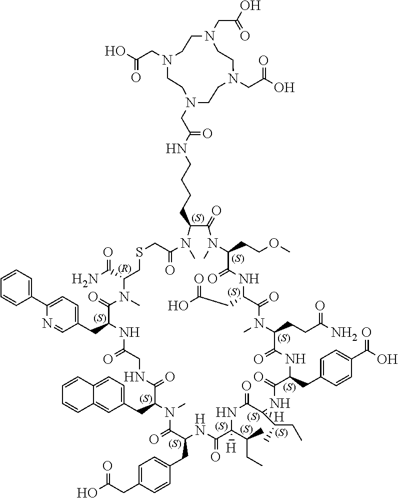 | C-21 (365) | 21 | 2203.03 | ESI-MS m/z: [M + 3H]3+ 735.8 |
| 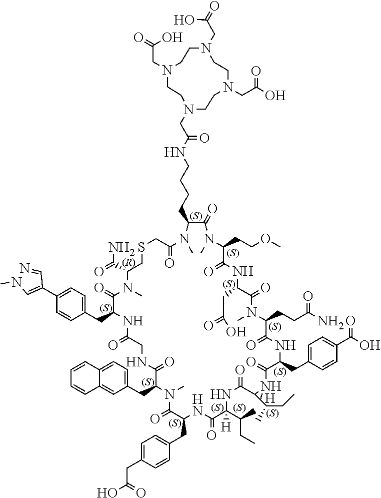 | C-22 (366) | 22 | 2206.0 | ESI-MS m/z: [M + 3H]3+ 736.8 |
| 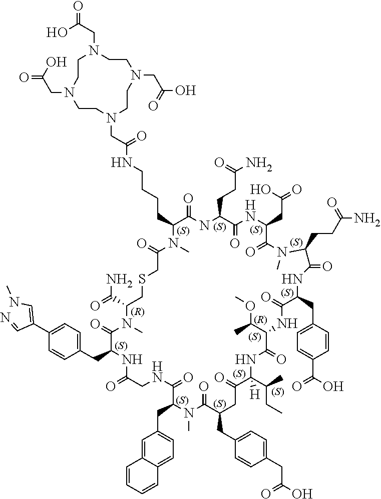 | C-24 (367) | 24 | 2221.01 | ESI-MS m/z: [M + 3H]3+ 741.8 |
| 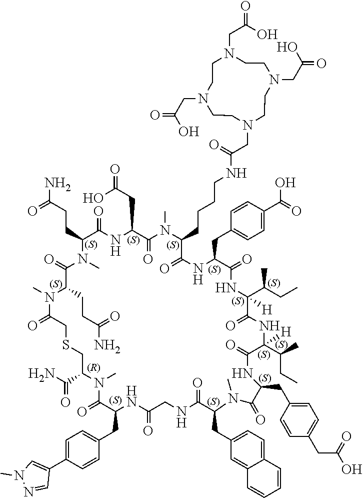 | C-26 (368) | 26 | 2219.04 | ESI-MS m/z: [M + 3H]3+ 741.2 |
| 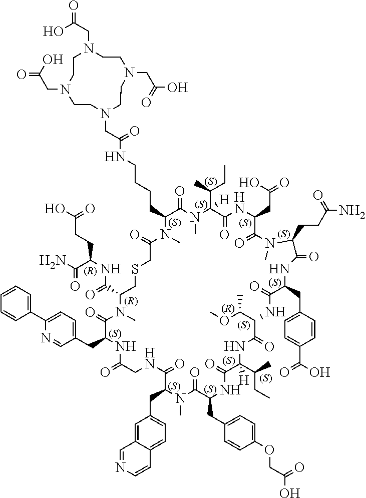 | C-31 (369) | 31 | 2349.07 | ESI-MS m/z: [M + 3H]3+ 784.5 |
| 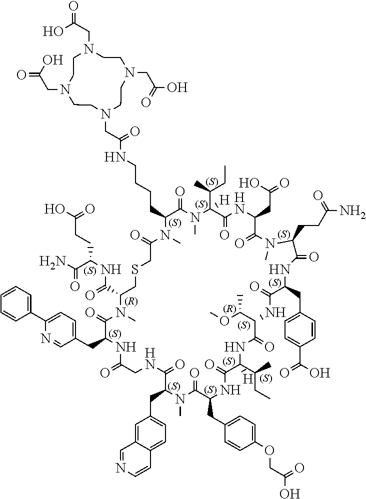 | C-32 (370) | 32 | 2349.07 | ESI-MS m/z: [M + 3H]3+ 784.5 |
| 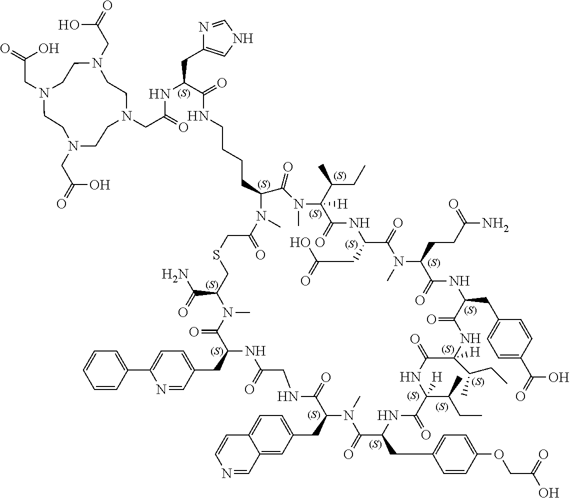 | C-34 (371) | 34 | 2355.10 | ESI-MS m/z: [M + 3H]3+ 786.5 |
| 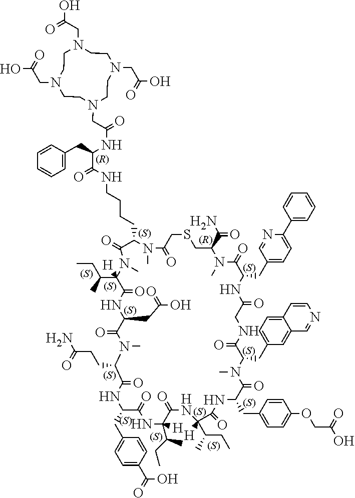 | C-41 (372) | 41 | 2365.11 | ESI-MS m/z: [M + 3H]3+ 789.9 |
| 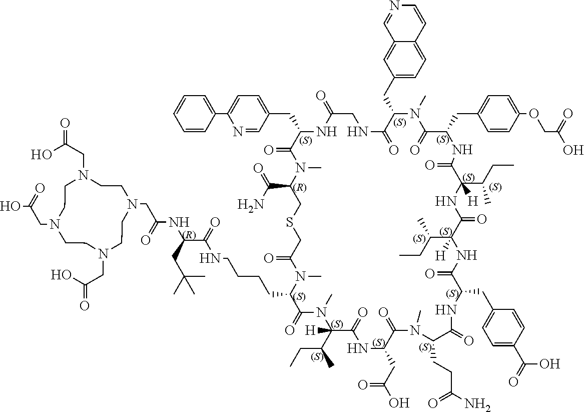 | C-42 (373) | 42 | 2345.14 | ESI-MS m/z: [M + 3H]3+ 783.2 |
| 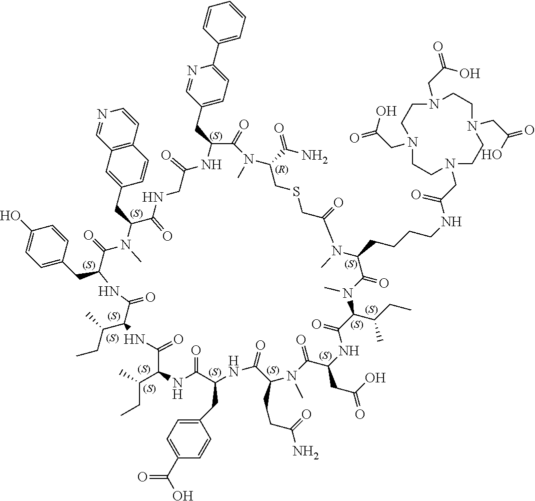 | C-1 (374) | 1 | 2160.04 | ESI-MS m/z: [M + 3H]3+ 721.5 |
| 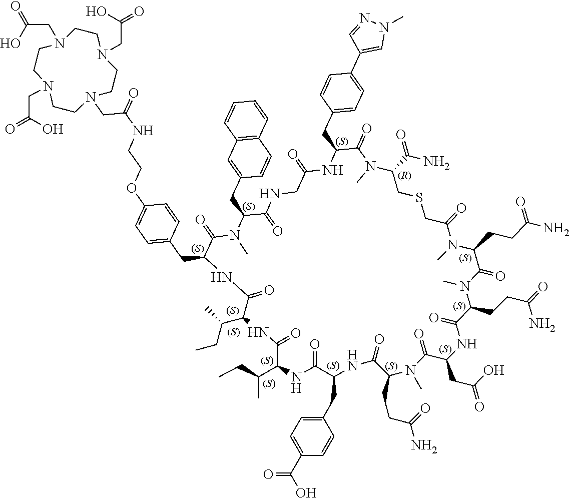 | C-28 (375) | 28 | 2220.04 | ESI-MS m/z: [M + 3H]3+ 741.5 |
| 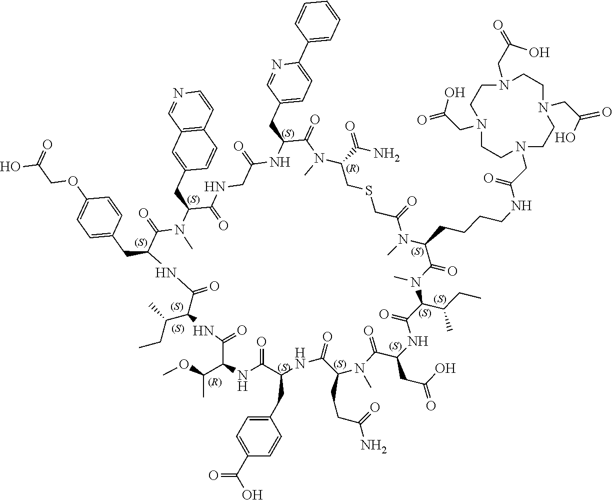 | C-30 (376) | 30 | 2220.02 | ESI-MS m/z: [M + 3H]3+ 741.5 |
| 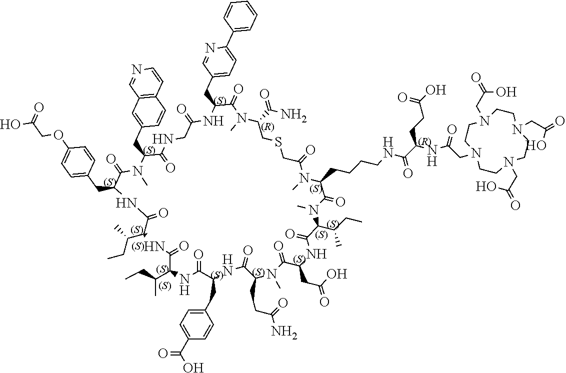 | C-33 (377) | 33 | 2347.09 | ESI-MS m/z: [M + 3H]3+ 783.9 |
| 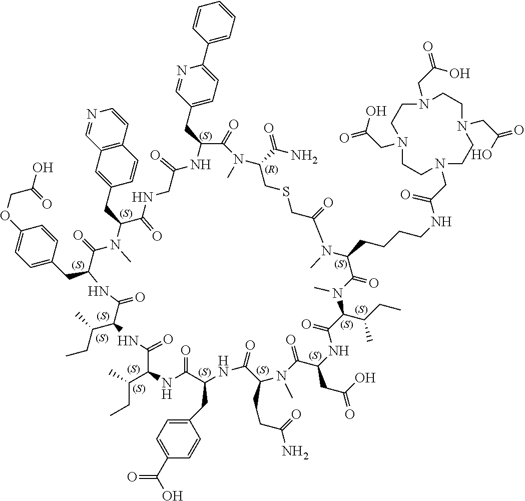 | C-64 (378) | 64 | 2218.04 | ESI-MS m/z: [M + 3H]3+ 740.8 |
| 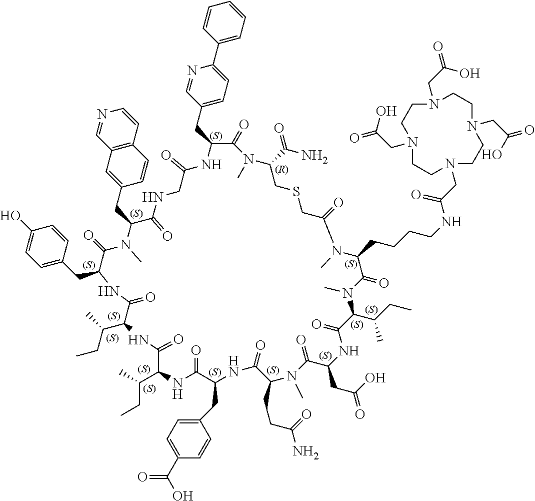 | C-62 (379) | 62 | 2162.02 | ESI-MS m/z: [M + 3H]3+ 722.2 |
| 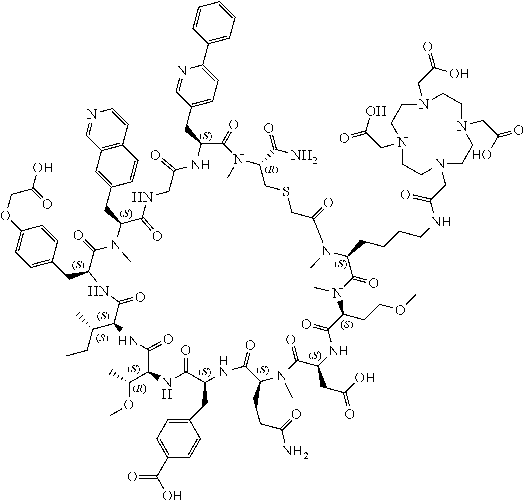 | C-63 (380) | 63 | 2222.00 | ESI-MS m/z: [M + 3H]3+ 742.2 |
| 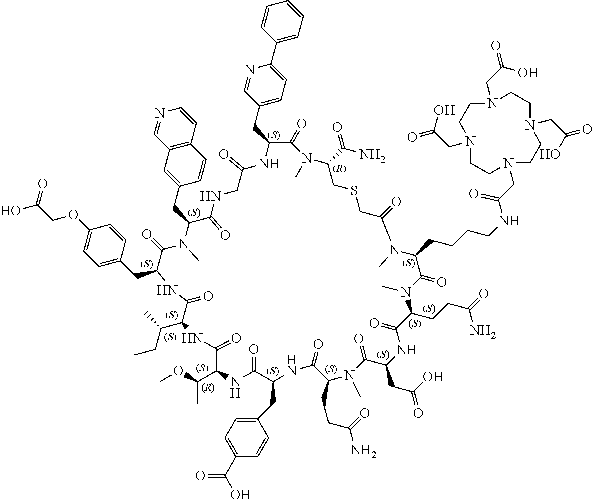 | C-35 (381) | 35 | 2235.00 | ESI-MS m/z: [M + 3H]3+ 746.3 |
| 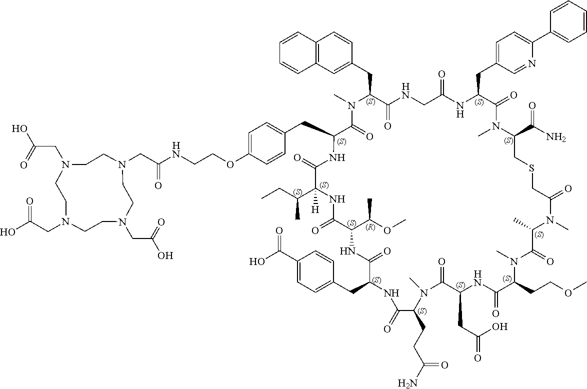 | C-44 (382) | 44 | 2148.99 | ESI-MS m/z: [M + 3H]3+ 717.7 |

### Example A5: Radiolabeling Protocols

### General Protocol for ¹⁷⁷Lu Radiolabeling of Empty-DOTA Conjugate

A 3 mCi aliquot of ¹⁷⁷LuCl₃ was added to the reaction vessel followed by addition of 10% ethanol in 0.4 M NaOAc buffer and the empty-DOTA conjugate. The resulting mixture was heated and mixed with a thermal mixer at 70° C. for 0.5 h. After 0.5 h, the reaction mixture was allowed to cool at room temperature for 2 min. Then the reaction mixture was diluted with a pre-mixed formulation buffer. To assess the radiochemical purity the ¹¹⁷Lu labeled product was injected on a rad-HPLC. At the end of the synthesis (EOS), the radiochemical purity was determined. HPLC column: Aeris 3.6 μm PEPTIDE XB-C18 100, LC Column, 150×4.6 mm (00F-4507-E0). Solvent A was H2O/0.05% TFA and solvent B was CH3CN/0.05% TFA. Elution was conducted at flow rate of 1.0 mL/min with gradient from 5 to 95% B over 16 min.

General protocol for ²²⁵Ac Radiolabeling of empty-DOTA conjugate

A 20 μCi aliquot of the ²²⁵AcCl₃ solution was added to a reaction vessel followed by addition of 10% ethanol in 0.4 M NaOAc buffer and the empty-DOTA conjugate. The resulting mixture was heated and mixed with a thermal mixer at 90° C. for 0.5 h. After 0.5 h, the reaction mixture was allowed to cool at room temperature for 1 min. The reaction mixture was diluted with a pre-mixed formulation buffer. The radiochemical purity was determined by radio thin-layer chromatography (radio-TLC). TLC plate: Agilent Technologies Chromatography paper (Cat number A201B12); mobile phase: 50 mM DTP A.

General protocol for ⁶⁴Cu Radiolabeling of empty-NOTA conjugate

A 5 mCi aliquot of ⁶⁴CuCl₂ solution was added to a reaction vessel followed by addition of 10% ethanol in 0.4 M NaOAc buffer and the empty-NOTA conjugate. The resulting mixture was heated and mixed with a thermal mixer at 70° C. for 0.5 h. After 0.5 h. the reaction mixture was allowed to cool at room temperature for 1 min. Then the reaction was diluted with a pre-mixed formulation buffer. To assess the radiochemical purity the product was injected on a rad-HPLC. At the end of the synthesis (EOS), the radiochemical purity was determined. HPLC column: Aeris 3.6 μm PEPTIDE XB-C18 100, LC Column, 150×4.6 mm (00F-4507-E0). Solvent A was H2O/0.05% TFA and solvent B was CH3CN/0.05% TFA. Elution was conducted at flow rate of 1.0 mL/min with gradient from 5 to 95% B over 16 min.

General protocol for ⁶⁶Ga Radiolabeling of empty-DOTA conjugate

A 9 mCi aliquot of ⁶⁸GaCl₃ was added to the reaction vessel followed by addition of 10% ethanol in 0.4 M NaOAc buffer and the empty-DOTA conjugate. The resulting mixture was heated and mixed with a thermal mixer at 90° C. for 0.17 h. After 0.17 h, the reaction mixture was allowed to cool at room temperature for 1 min. Then the reaction was diluted with a pre-mixed formulation buffer. To assess the radiochemical purity the product was injected on a rad-HPLC. At the end of the synthesis (EOS), the radiochemical purity was determined. HPLC column: Aeris 3.6 μm PEPTIDE XB-C18 100, LC Column, 150×4.6 mm (00F-4507-E0). Solvent A was H2O/0.05% TFA and solvent B was CH3CN/0.05% TFA. Elation was conducted at flow rate of 1.0 mL/min with gradient from 5 to 95% B over 16 min.

### Example A6: Synthesis of 2²Ac-C-30

²²⁵Ac-C-30 was synthesized according to the corresponding radiolabeling protocols described in Example A5. The nat-La chelate standard and ²²⁵Ac chelate eluted at the same retention time, confirming successful labeling. Successful ²²⁵Ac labeling of ²²⁵Ac-C-62 was also confirmed according to Example A6.

### Example A7: Synthesis of ⁶⁸Ga-C-30

⁶⁸Ga-C-30 was synthesized according to the corresponding radiolabeling protocols described in Example A5. The nat-Ga chelate standard and ⁶⁸Ga chelate eluted at the same retention time, confirming successful labeling.

### Example A8: Synthesis of ¹⁷⁷Lu-C-30

¹⁷⁷Lu-C-30 was synthesized according to the corresponding radiolabeling protocols described in Example A5 The nat-Lu chelate standard and ¹⁷⁷Lu chelate eluted at the same retention time, confirming successful labeling. Successful ¹⁷⁷Lu c labeling of ¹⁷⁷Lu-C-1, ¹⁷⁷Lu-C-28, ¹⁷⁷Lu-C-33, ¹⁷⁷Lu-C-35, ¹⁷⁷Lu-C-45, ¹⁷⁷Lu-C-62, ¹⁷⁷Lu-C-64, and ¹⁷⁷Lu-C-72 was also confirmed according to Example A8.

### Example A9: Synthesis of ⁶⁴Cu-C-30NOTA

⁶⁴Cu-C-30NOTA was synthesized according to the corresponding radiolabeling protocols described at example A5. The nat-Cu chelate standard and ⁶⁴Ca chelate eluted at the same retention time, confirming successful labeling.

### Example B. Synthesis of Peptides

### Analytical Methods, Materials, and Instruments.

Unless otherwise noted, reagents and solvents were used as received from commercial suppliers. If not otherwise specified, purity and low-resolution mass spectral data were measured using a Shimadzu Nexera XR system coupled to a LCMS-2020 MS (ESI). Methods are specified below.
Method A-1 (HPLC-MS): Phenomenex Kinetex EVO C18 2.6 μm, 2.1ID×150 mm, 100 Å (with a guard cartridge 2.1 mmID); 60° C.; 0.5 mL/min; (A) H₂O+0.025% TFA/(B) MeCN+0.025% TFA; gradient: from 5 to 45% B in 7.2 min. Detection: electrospray mass spectra (+) and PDA-UV at UV of 225 nm.
Method A-2 (HPLC-MS): Phenomenex Kinetex EVO C18 2.6 μm, 2.1ID×150 mm, 100 Å (with a guard cartridge 2.1 mmID); 60° C.; 0.5 mL/min; (A) H₂O+0.025% TFA/(B) MeCN+0.025% TFA; gradient: from 20 to 60% B in 7.2 min, Detection: electrospray mass spectra (+) and PDA-UV at UV of 225 nm.

### General Preparative HPLC Purification Method.

Crude containing the desired polypeptide were purified by preparative RP-HPLC using DAD-UV chromatogram at UV 220 nm and electrospray mass spectra (+) for the detection and employing commercially available RP-HPLC columns such as Waters XBridge CIS, Waters XSelect C18, Phenomenex Kinetex EVO C18, and Interchim US5C18HQ-100/300. Otherwise specified, (A) H₂O+0.1% TFA and (B) MeCN+0.1% with varying gradients were employed as eluents. Product-containing fractions were collected and lyophilized to obtain the purified product.

### General Synthetic Aspects.

Macrocyclic peptides type-A can be synthesized as outlined in Scheme-P1.

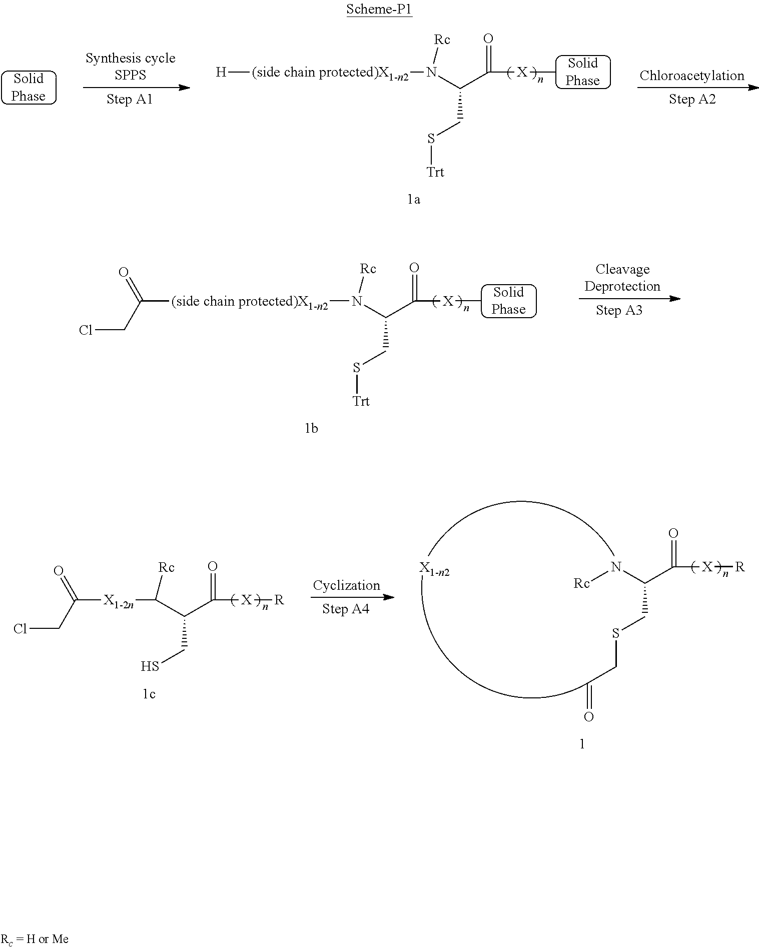

Step-A1: Peptide elongation on resin.

The peptide elongations in this disclosure were performed in automated peptide synthesizers employing conditions as described in Method-S1, Method-S2, and Method-S3 in accordance with the manufacturer's instruction. In addition, the peptide coupling with certain Fmoc-AAs was performed by the synthesizer or by manually as described in the Table S1-1. In detail, Fmoc-Sieber amide resin was suspended in the solvent (e.g., DMF or DCM) and then loaded onto the peptide synthesizer. After Fmoc removal of the resin, Fmoc-AA coupling and subsequent Fmoc removal as one cycle was performed employing conditions suitable for each Fmoc-AA as described below in Table S1-2. The peptide elongation on resin was continued until the desired linear polypeptide sequence 1 a was obtained. Below described the AA coupling and Fmoc deprotection conditions.

Method S1 employing Liberty BLUE HT 12TM (CEM. Inc.).
General AA coupling conditions

TABLE S1-1

| Fmoc-AA | Reagents | Coupling conditions |
|---|---|---|
| Fmoc-MeC(Trt)-OH | DIC/Oxyma Pure ® | 50° C., 1-2x 15-20 min; |
| | in DMF or NMP | or 40° C., |
| | | 1-2x 30 min; or 25° C., |
| | | 1-2x 30 min |
| Fmoc-H(Trt)-OH | | 50° C., 2x 30 min |
| Other than listed above | | 90° C., 1-2x 3-10 min or |
| | | 75° C., 1-2x 10-30 min |

Fmoc deprotection on resin.

TABLE S1-2

| Fmoc-AA on peptide | Reagents | Deprotection conditions |
|---|---|---|
| Fmoc-AA coupled with | pyrrolidine/DMF (1/9) | 25° C., 1-2x 1-5 min or 25° C., |
| N-Me-AA | or piperidine/DMF (1/4) | 5 min then 25° C., 15 min |
| Other than listed above | | 90° C., 1 min or 75° C., 3 min or |
| | | 50° C., 1.5 min |

Method S2 employing Liberty Prime (CEM. Inc.).
General AA coupling on resin.

TABLE S2-1

| Fmoc-AA | Reagents | Coupling conditions |
|---|---|---|
| Fmoc-MeC(Trt)-OH | DIC/Oxyma Pure ® | 50° C., 1-2x 15-20 min or |
| | in DMF or NMP | 40° C., 1-2x 30 min |
| Other than listed above | | 105° C., 1-2x 1-2 min; or |
| | | 90° C., 1-2x 3-10 min; or |
| | | 75° C., 2x 20-30 min |

General method for Fmoc deprotection

TABLE S2-2

| Fmoc-AA on peptide | Reagents | Deprotection conditions |
|---|---|---|
| Fmoc-AA coupled with | pyrrolidine/DMF (1/9) | 25° C. 1-2x 1-5 min |
| N-Me-AA | | |
| Other than listed above | 83 mM Oxyma Pure ® in | 110° C., 1-1.5 min |
| | pyrrolidine/DMF (4/96) | |

Method S3 employing Cyro I (Biotage. Inc.).
General AA coupling

TABLE S3-1

| Fmoc-AA | Reagents | Coupling conditions |
|---|---|---|
| Fmoc-MeC(Trt)-OH | HATU/DIEA in | 25° C., 40 min |
| Other than listed | DMF or NMP | 75° C., 2x 20-30 min |
| above | | |

### General Fmoc Deprotection

TABLE S3-2

| Fmoc-AA on peptide | Reagents | Deprotection conditions |
|---|---|---|
| All AAs | piperidine/DMF (1/4) | 25° C., 5 min then 25° C., 15 min |

Method 4: Genera peptide coupling without automate synthesizers.

The peptide coupling with the AAs listed below were performed manually without microwave employing conditions in Table S4. (Note: the AAs listed below can be introduced by the peptide synthesizer employing above methods as well).

TABLE S4

| Fmoc-AA | Reagents | Coupling conditions |
|---|---|---|
| Fmoc-MeNal27N-OH | HATU/DIEA (3-4.2/3-4/6-12) in NMP | 25° C., 1x 30-60 min |
| Fmoc-SR3Et-OH | HATU/DIEA (3/3/6) in DMF | 25° C., 1x 40 min |
| Fmoc-MeA4paa(tBu)-OH | DIC/Oxyma Pure ® (3/4/8) in DMF | 75° C., 1x 30 min |

### Step A2: Chloroacetylation.

The obtained polypeptide 1a on resin was transferred in a syringe with a flit. The resin was shaken in a reagent mixture (see below) at room temperature for 0.5-2 h. The solution was then drained through the frit. The resin was washed successively a few times with DMF, DCM, and Et₂O to afford the linear peptides 1b on resin.
Reagent mixture: resin/ClAcOH in DMF(0.087-0.25M)/HATU in DMF(0.087-0.25M)/DIPEA in DMF (0.17-0.5M)=1 eq/4.2-5 eq/4-5 eq/4.2-10 eq.
   Step A3: Cleavage of polypeptides from the resin along with the global deprotection of side chain protecting groups (PGs).

Polypeptide 1b on resin was shaken at rt for 5-90 min in the cleavage cocktail (TFA/TIS/DODT/H₂O=92.5/2.5/2.5/2.5). The resin was filtered and rinsed with the cocktail used. Combined filtrates were poured into cold diethyl ether. The resulting suspension was centrifuged (9000 rpm, 1 min at 0° C.), and then the supernatant was decanted out. The precipitate was suspended in cold ether, vortexed briefly, and then centrifuged. This process was repeated a few times when appropriate and necessary. The crude containing polypeptide 1c was dried tinder reduced pressure.

Step A4: Peptide cyclization and purification.

The crude containing polypeptide 1c was dissolved in MeCN/H₂O (1/1). To the solution was added TEA (5-20 eq), and then the mixture was stirred for 1 hr to 18 hrs until completion of the reaction. The resulting mixture was rendered acidic by AcOH, and then reaction mixture was concentrated by Genevac EZ-2 Elite or HT-12. The resulting residue was purified by RP-HPLC to afford Macrocyclic peptide type-A.

Macrocyclic peptides type-B in the present disclosure can be synthesized as outlined in Scheme-P2. Note that the following methods can afford desired sidechain branched polypeptide at any AA position by appropriate order of Fmoc-AA used.

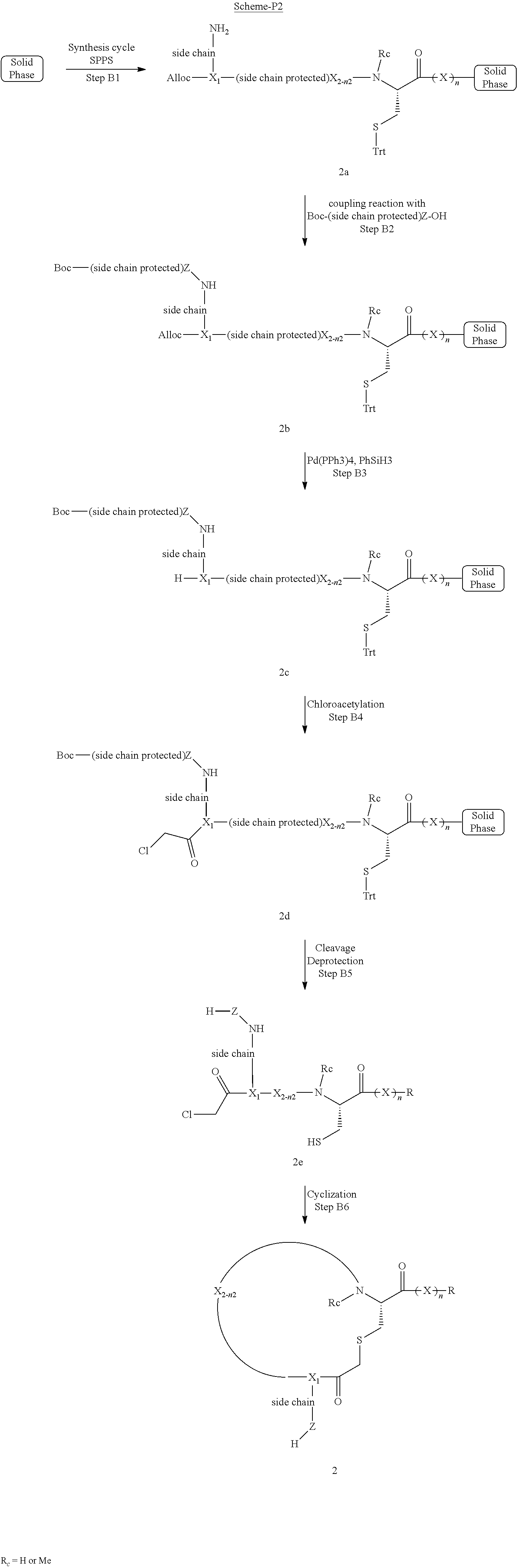

Step B1: Polypeptides type 2a on-resin were synthesized in the fashion analogous to Step-A1 in Scheme-P1 but employing appropriately protected AA building blocks such as Alloc-K(Fnoc)-OH. Subsequently, treatment of 2a with one of the Fmoc deprotection conditions above, followed by the coupling with the Boc-protected AAs employing the described condition in Table S-5 afforded 2b.

TABLE S-5

| Boc-protected AAs | | |
|---|---|---|
| (the side chain | | |
| protected properly | Reagents | Coupling conditions |
| Boc-df-OH | HATU/DIEA (3/3/6) in DMF | 25° C., 30 min (coupling |
| | | manually) |
| Boc-H(Trt)-OH | DIC/Oxyma Pure ® (4.2/8/4) in | 50° C., 2x 30 min (Liberty Blue) |
| | DMF | |
| Boc-de(tBu)-OH | HATU/DIEA (3/3/6) in DMF | 75° C., 2x 30 min (Liberty Blue) |

Step B3: Deprotection of the Alloc protecting group

Polypeptides 2b on resin were swollen in DCM. The resin was shaken with Pd(PPh₃)₄(0.2-0.25 eq) and PhSiH₃ (10-15 eq) at room temperature for 1 h. The resin was washed with DMF. Then the resin was washed by DCM followed by DMF to afford polypeptides 2c on resin.

### Step B4, -B5, and -B6:

Subsequently, polypeptide 2c on resin were transformed to Macrocyclic peptides type-B by similar fashion analogous to the preparation in Scheme-P1.

Macrocyclic peptides type-C in the present disclosure can be synthesized as outlined in Scheme-P3.

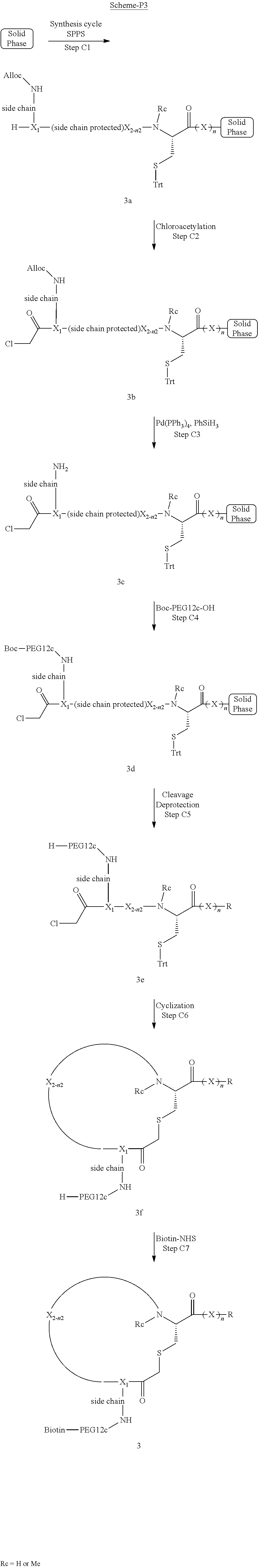

### Step C1, C2, and C3:

Polypeptides 3c were synthesized by methods analogously to the preparation above employing appropriate Fmoc-AA building blocks.

Step C4: elongation of the side chain with Boc-PEG12c-OH.

Polypeptides 3c on resin were suspended in DMF. The resin was shaken with Boc-PEG12c-OH (4.2-5.3 eq)/HATU (4-5 eq)/DIEA (8-10 eq) at room temperature for 30 min; or Boc-PEGI2c-OH (4.2-5.3 eq)/PyAOP (4-5 eq)/DIEA (8-10 eq) at 40° C. for 30 min. The resin was washed with DMF. Then the resin was washed by DCM followed by DMF to provide polypeptides 3d on resin.

### Step C5 and C6:

Polypeptides 3f were synthesized from polypeptides 3d on resin in the fashion analogous to Scheme-PL.

Step C7: Coupling reaction with Biotin-OSu

To the polypeptides 3f in DMSO were added Biotin-OSu (approx. 1 eq) and DIEA (3-5 eq). The mixture was shaken at room temperature for 0.2-1 h. The reaction mixture was directly purified by preparative RP-HPLC to afford Macrocyclic peptides type-C.

Macrocyclic peptide-conjugates type-D, when one of AA sidechains contains a nucleophilic amine, in the present disclosure can be synthesized outlined in scheme-P4.

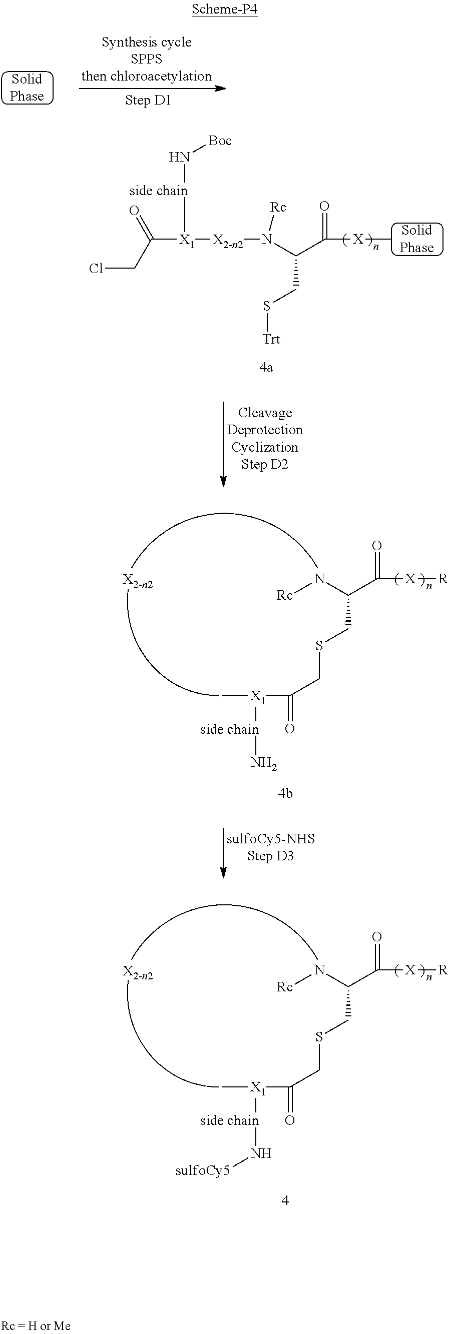

Polypeptides 4a on resin were synthesized by methods described above. Subsequently, the cleavage from the resin along with the global deprotection, followed by the cyclization and then RP-HPLC purification were performed similarly by the methods described above to afford polypeptide 4b. Finally, treatment of 4b with sulfoCy5-NHS, followed by the RP-HPLC purification afforded Macrocyclic peptide-conjugates type-D.

Alternatively, the crude containing 4b was treated with sulfoCy5-NHS, followed by RP-HPLC afforded Macrocyclic peptide-conjugates type-D.

Macrocyclic peptide-conjugates type-E can be synthesized via appropriate AA sequencing as shown in Scheme-P5.

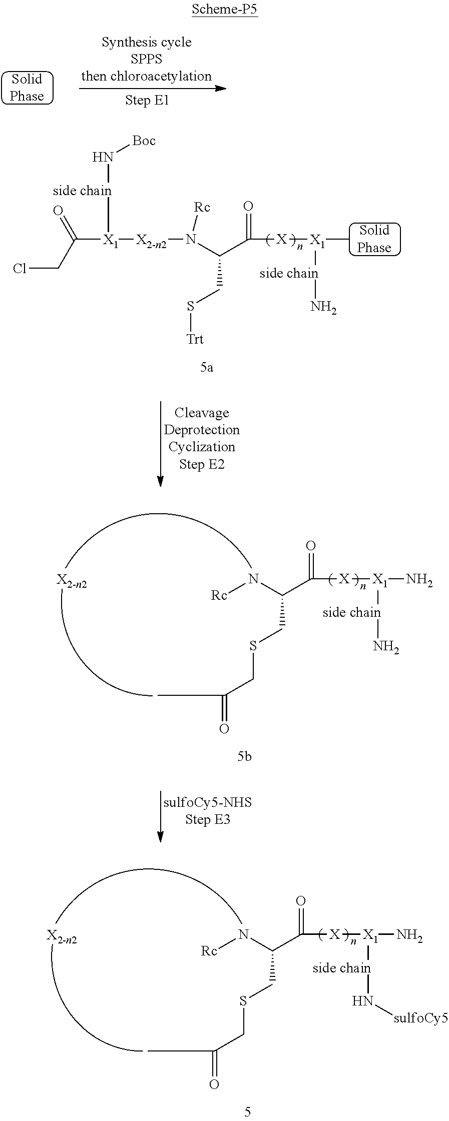

Polypeptides 5b were synthesized by methods described above via polypeptides 5a on resin. After the purification of 5b, treatment with sulfoCy5-NHS, followed by the RP-HPLC purification afforded Macrocyclic peptide-conjugates type-E.

Macrocyclic peptide type-F can be synthesized as outlined in Scheme-P6.

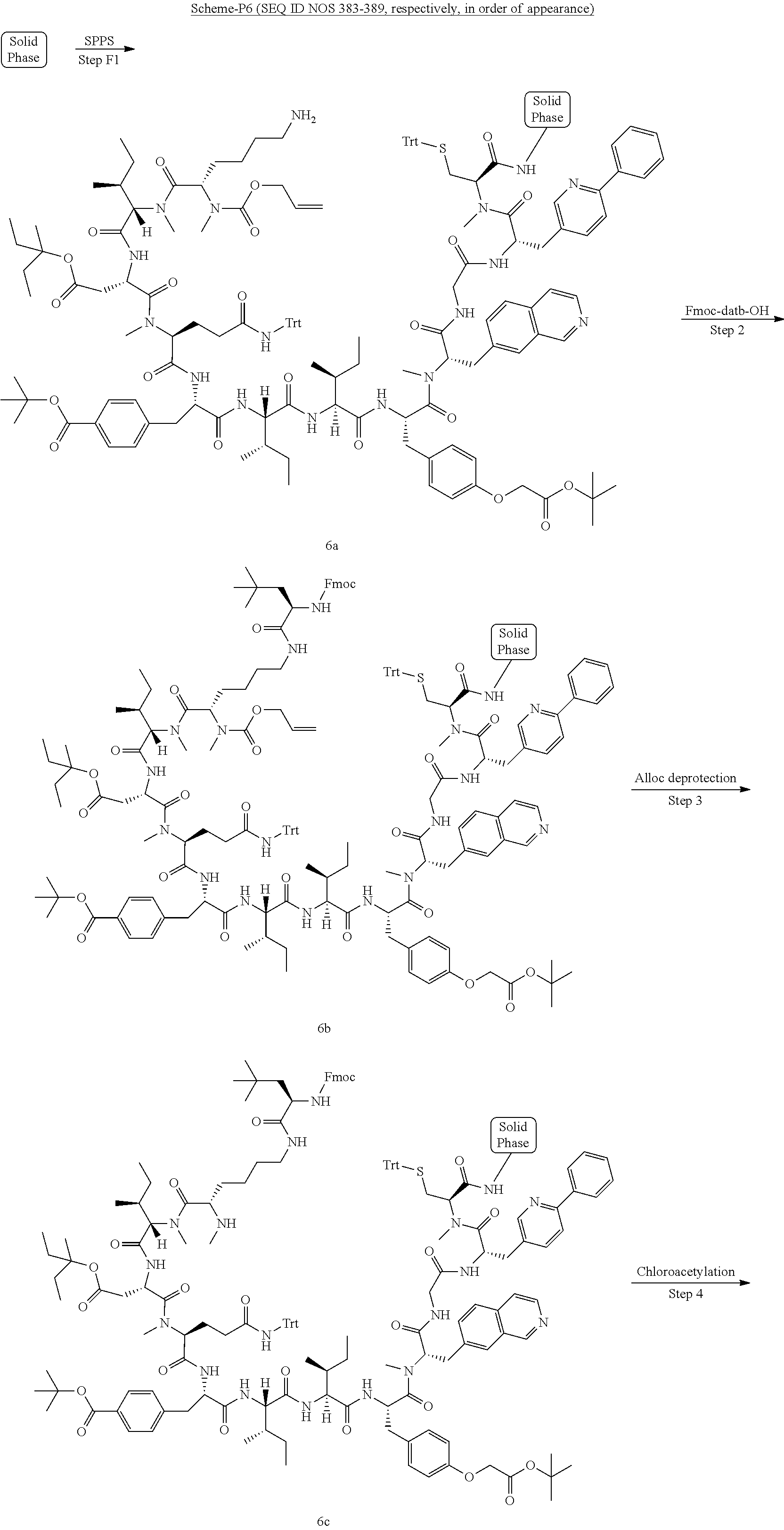

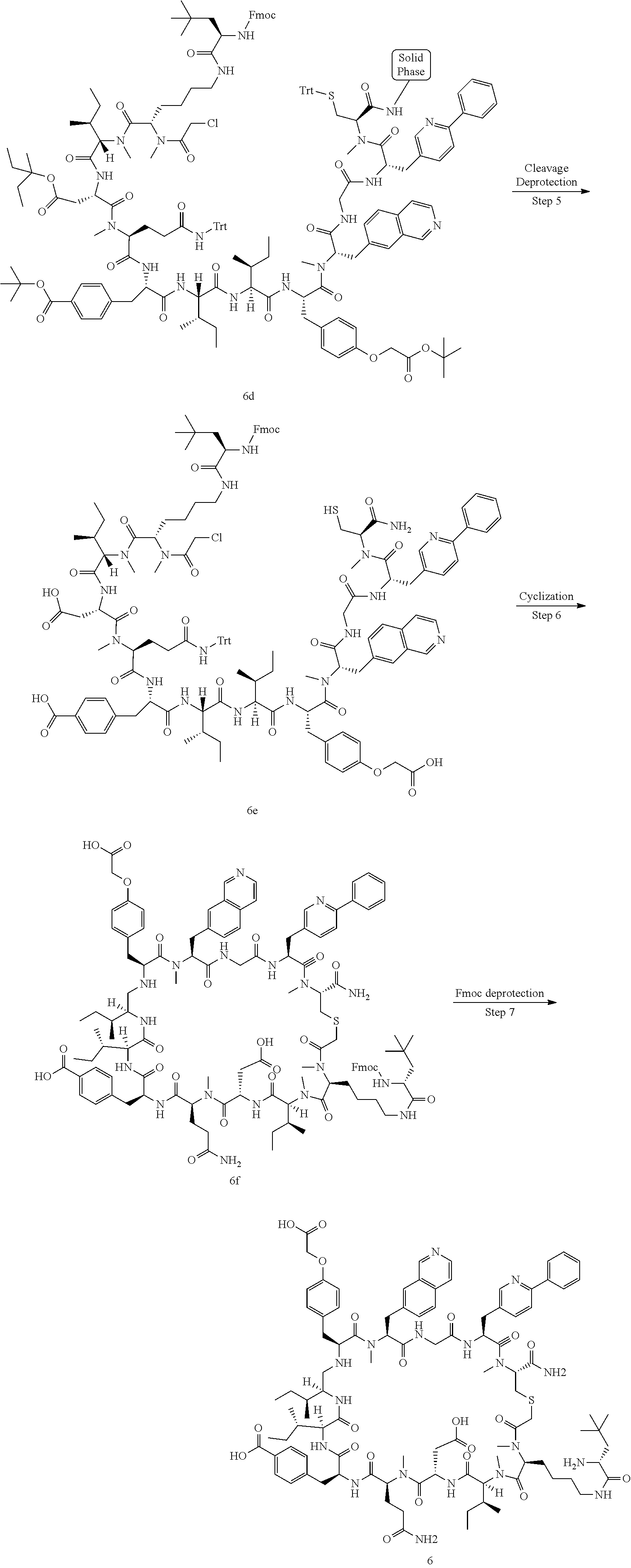

Polypeptides type 6b via 6a on-resin were synthesized in the fashion analogous to Step-A1 in Scheme-P1 but employing appropriately protected AA building blocks such as Alloc-K(Fmoc)-OH. Subsequently, Alloc deprotection and the chloroacetylation described in Step-B3 and Step-A2 respectively can afford linear polypeptide type 6d on resin. Cleavage from the resin along with the removal of the acid labile protecting groups by methods described in Step-A3 can afford 6e, which was then treated for the nacrocyclization by methods described in such as Step-A4 can afford 6f. Removal of the Fmoc group of 6f can be achieved by treatment with suitable bases such as pyrrolidine in solvent such as DMF to afford Macrocyclic peptide type 6.

### Example B1: (SEQ ID NO: 363)

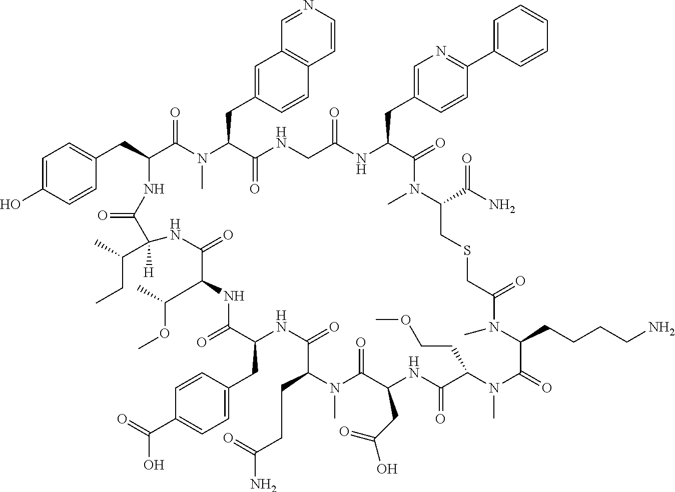 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC1=O]

The desired polypeptide was synthesized as outlined in Scheme-P1. The linear polypeptide was synthesized on Fmoc-Sieber amide resin (0.25 mmol) using Fmoc-AAs listed in below table. Method S-1 was employed for the peptide elongation. The obtained peptide on the resin was subjected to the chloroacetylation described in Step-A2 (CIAcOH (1.25 mmol)/HATU/DIEA (2.5 mmol) in DMF (12.5 mL), 30 min), followed by treating with the cleavage cocktail (12 mL) for 5 min as outlined in Step-A3 to furnish the linear peptide. The crude containing the linear peptide (0.25 mmol as theoretical based on the resin used) was dissolved in CH₃CN/H₂O (100 mL), and then added Et₃N (2.5 mmol, 348 μL), and then shaken for at room temperature for 18 hrs, and then concentrated as outlined in Step-A4. The resulting residue was purified by preparative RP-HIPLC. Pure fractions were combined and lyophilized to afford the title macrocyclic peptide. HPLC-MS (Method A-1): 4.67 min. ESI-MS m/z: 890.7 [M+2H]2+.

TABLE S-6

| order of Fmoc-AA | AA position in | |
|---|---|---|
| used in the SPPS | the cyclic peptide | Fmoc-AA |
| 1 | X12 | Fmoc-MeC(Trt)-OH |
| 2 | X11 | Fmoc-3Py6Ph-OH |
| 3 | X10 | Fmoc-G-OH |
| 4 | X9 | Fmoc-MeNal27N |
| 5 | X8 | Fmoc-Y(tBu)-OH |
| 6 | X7 | Fmoc-I-OH |
| 7 | X6 | Fmoc-TMe-OH |
| 8 | X5 | Fmoc-F4COO(tBu)-OH |
| 9 | X4 | Fmoc-Me(Trt)-OH |
| 10 | X3 | Fmoc-D(Mpe)-OH |
| 11 | X2 | Fmoc-MeHseMe-OH |
| 12 | X1 | Fmoc-MeK(Boc)-OH |

### Example 132: The Following Peptides were Synthesized by Similar Methods Described Above

TABLE 10

| Example | Sequence | | Analytical |
|---|---|---|---|
| No. | ID NO: | Structure | data |
| 2-1 | 2 | 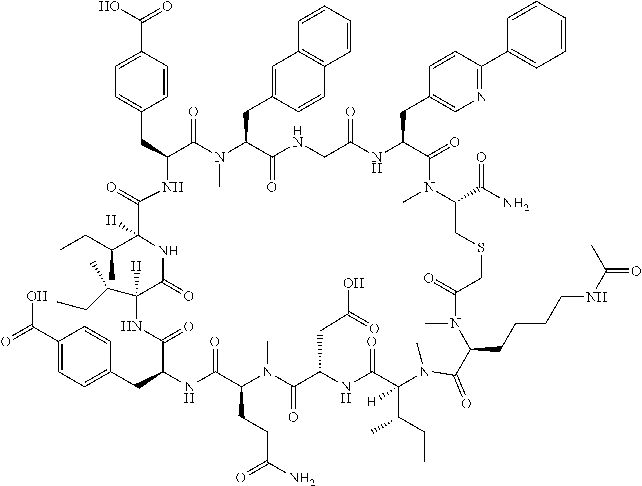 | HPLC-MS (Method A-2): 5.04 min ESI-MS m/z: 923.3 [M + 2H]2+ |
| 2-2 | 3 | 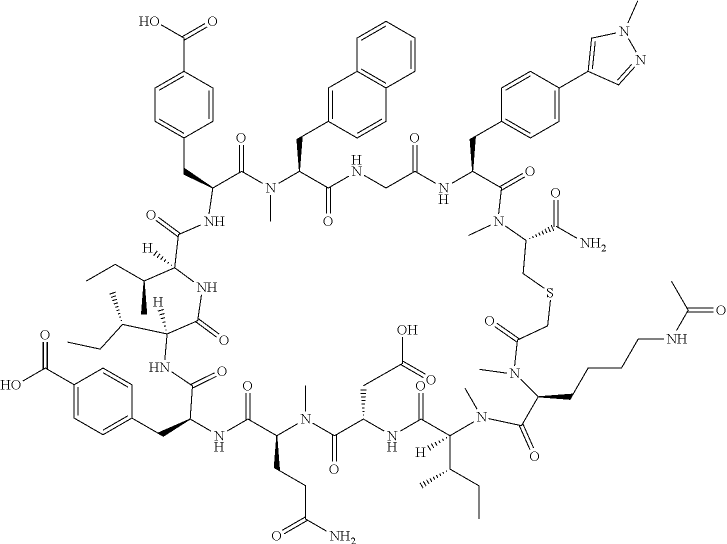 | HPLC-MS (Method A-2): 5.66 min ESI-MS m/z: 924.9 [M + 2H]2+ |
| 2-3 | 4 | 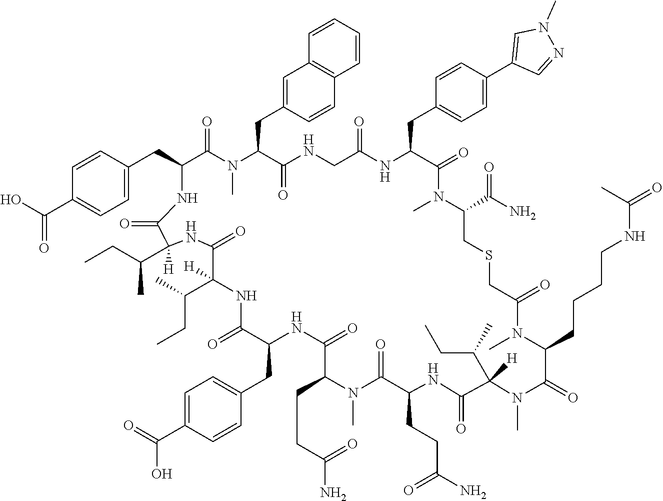 | HPLC-MS (Method A-2): 5.55 min ESI-MS m/z: 931.4 [M + 2H]2+ |
| 2-4 | 7 | 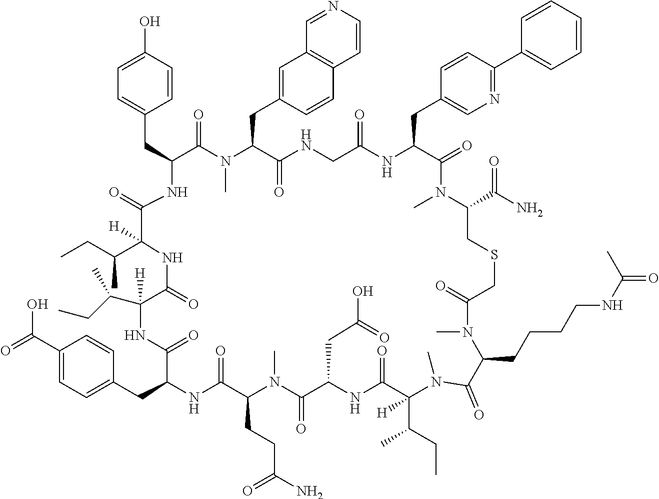 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(C)=O)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O] | HPLC-MS (Method A-2): 3.47 min ESI-MS m/z: 909.4 [M + 2H]2+ |
| 2-5 | 8 | 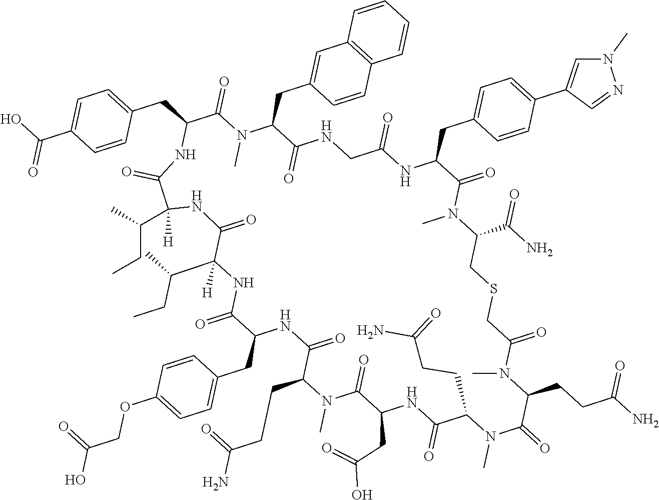 | HPLC-MS (Method A-2): 4.96 min ESI-MS m/z: 911.3 [M + 2H]2+ |
| 2-6 | 9 | 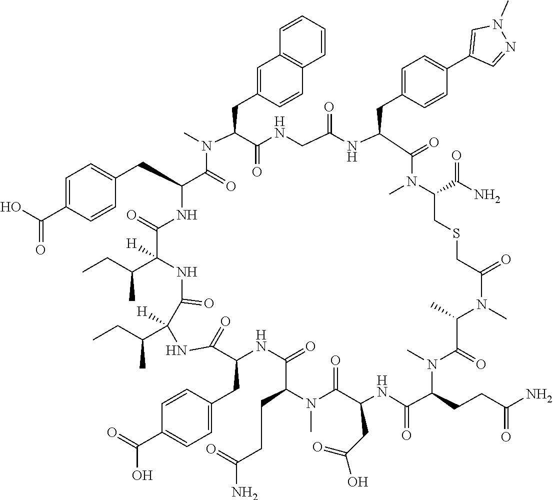 | HPLC-MS (Method A-2): 5.20 min ESI-MS m/z: 882.74 [M + 2H]2+ |
| 2-7 | 10 | 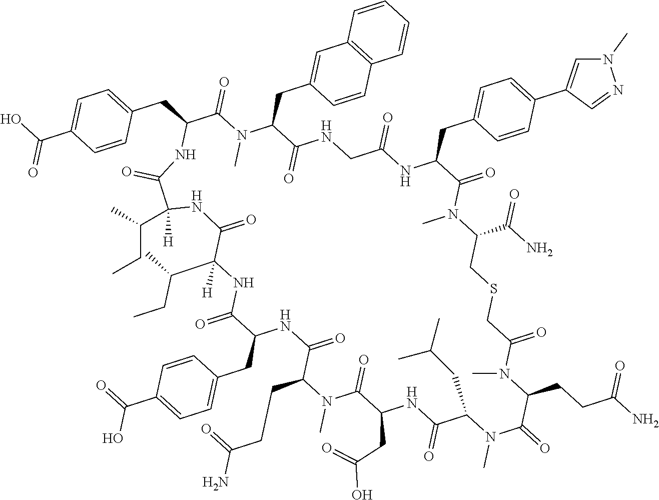 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CC(C)C)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 5.80 min ESI-MS m/z: 903.5 [M + 2H]2+ |
| 2-8 | 11 | 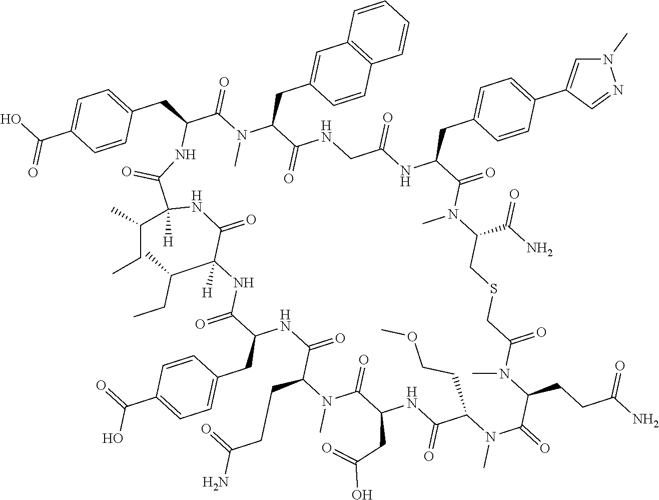 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 5.50 min ESI-MS m/z: 904.5 [M + 2H]2+ |
| 2-9 | 12 | 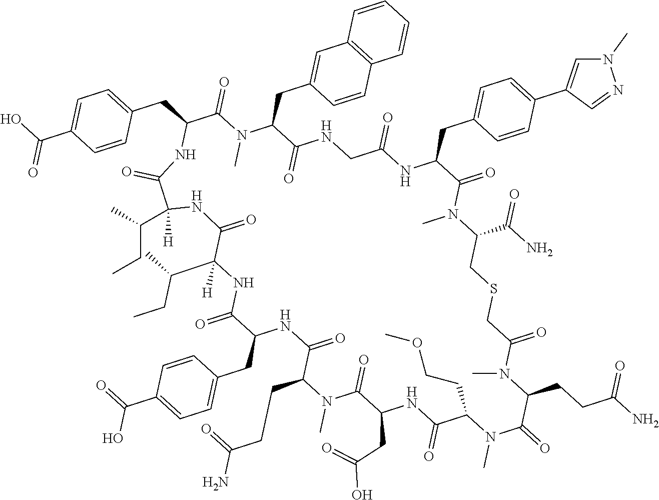 | HPLC-MS (Method A-2): 5.40 min ESI-MS m/z: 897.4 [M + 2H]2+ |
| 2-10 | 13 | 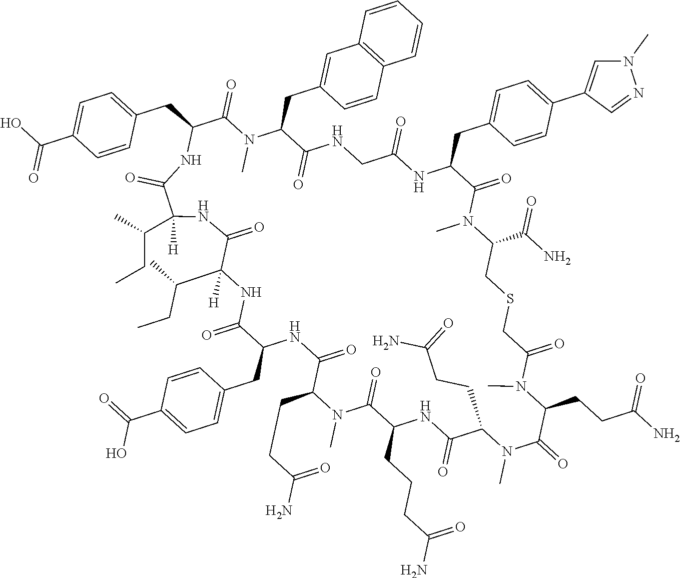 | HP:C-MS (Method A-2): 4.84 min ESI-MS m/z: 924.8 [M + 2H]2+ |
| 2-11 | 14 | 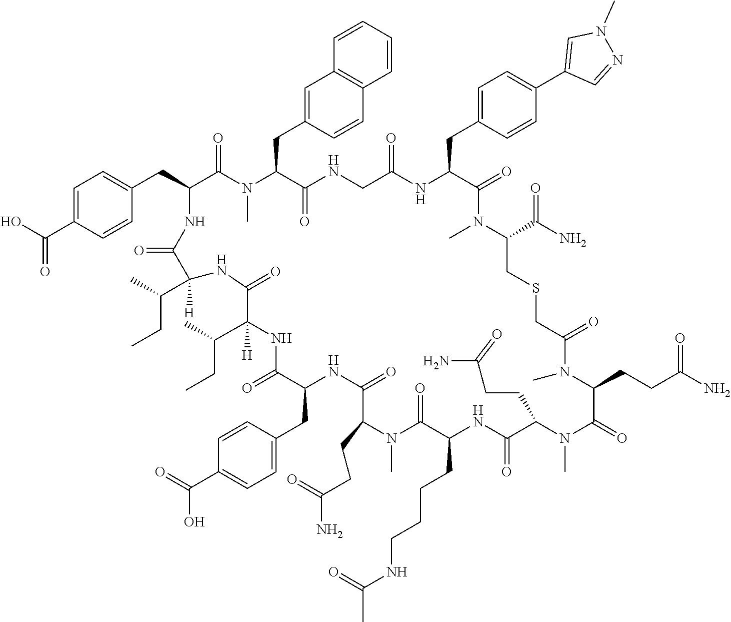 [SMILES: CC[C@H](C)[C@@H]1NC(=O)[C@H](Cc2ccc(C(=O)O)cc2)NC(=O)[C@H](CCC(N)=O)N(C)C(=O)[C@H](CCCCNC(C)=O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 5.34 min ESI-MS m/z: 938.5 [M + 2H]2+ |
| 2-12 | 15 | 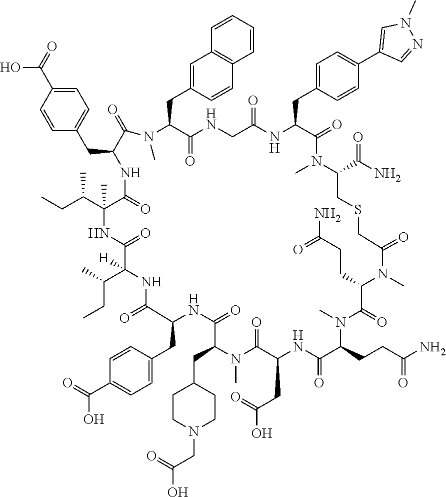 | HPLC-MS (Method A-2): 4.58 min ESI-MS m/z: 953.3 [M + 2H]2+ |
| 2-13 | 16 | 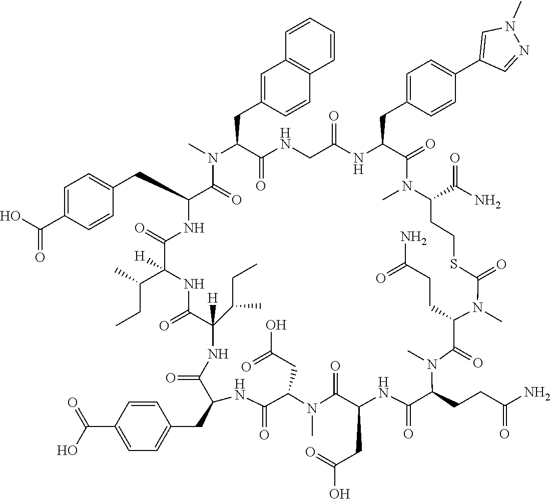 [SMILES: CC[C@H](C)[C@@H]1NC(=O)[C@H](Cc2ccc(C(=O)O)cc2)NC(=O)[C@H](CC(=O)O)N(C)C(=O)[C@H](CC(=O)O)NC(=O)[C@H](CCC(N)=O)N(C)C(=O)[C@H](CCC(N)=O)N(C)C(=O)SCC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 5.40 min ESI-MS m/z: 904.4 [M + 2H]2+ |
| 2-14 | 17 | 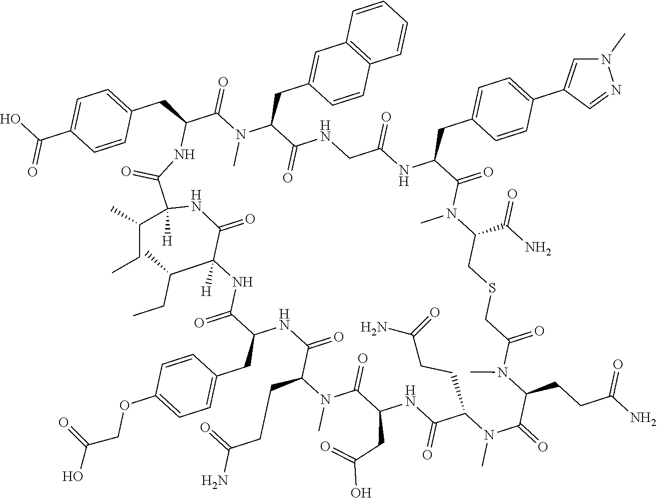 [SMILES: CC[C@H](C)[C@@H]1NC(=O)[C@H](Cc2ccc(OCC(=O)O)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 5.30 min ESI-MS m/z: 926.0 [M + 2H]2+ |
| 2-15 | 18 | 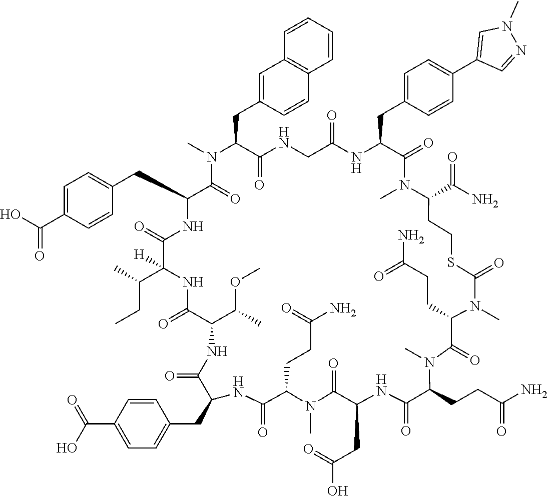 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)SCC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC1=O] | HPLC-MS (Method A-2): 5,02 min ESI-MS m/z: 911.9 [M + 2H]2+ |
| 2-16 | 19 | 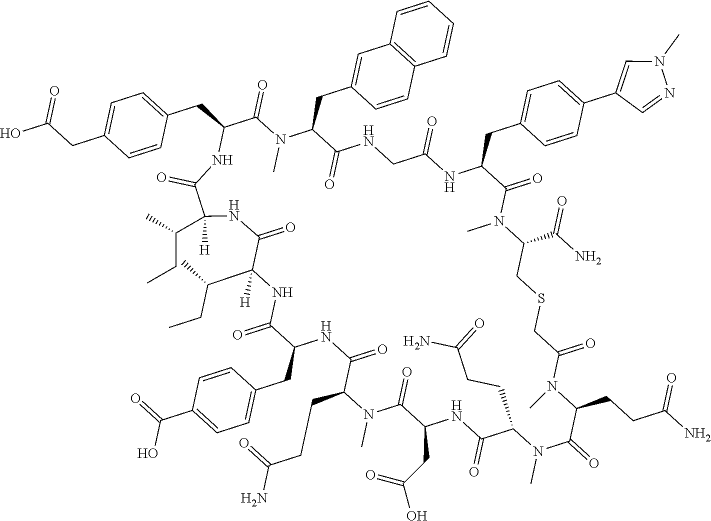 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(CC(=O)O)cc2)NC(=O)[C@H]([C@@H](C)CC)NC1=O] | HPLC-MS (Method A-2): 5.32 min ESI-MS m/z: 917.9 [M + 2H]2+ |
| 2-17 | 20 | 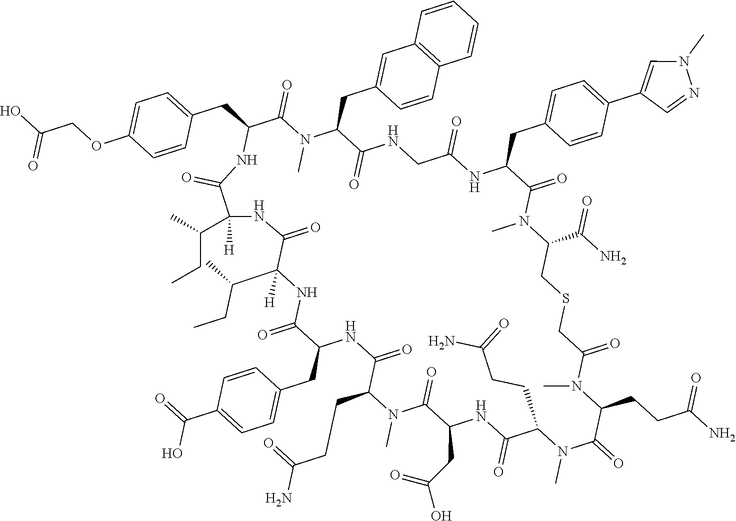 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(OCC(=O)O)cc2)NC(=O)[C@H]([C@@H](C)CC)NC1=O] | HPLC-MS (Method A-2): 5.25 min ESI-MS m/z: 925.9 [M + 2H]2+ |
| 2-18 | 36 | 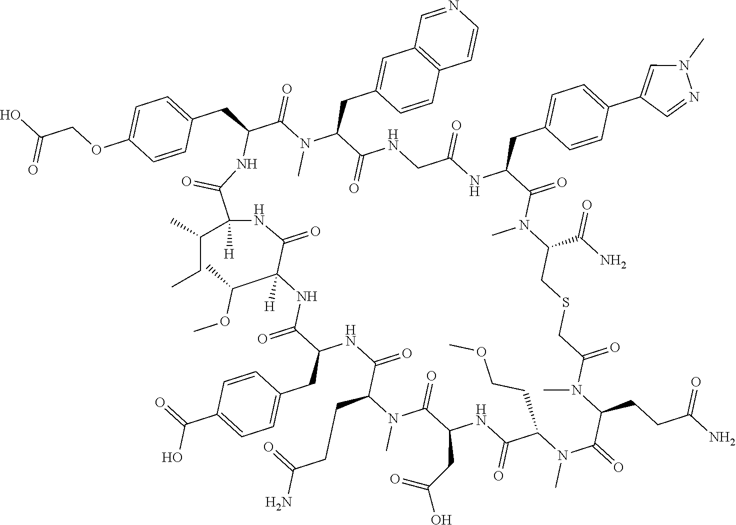 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccncc3c2)N(C)C(=O)[C@H](Cc2ccc(OCC(=O)O)cc2)NC1=O] | HPLC-MS (Method A-1): 5.88 min ESI-MS m/z: 920.9 [M + 2H]2+ |
| 2-19 | 37 | 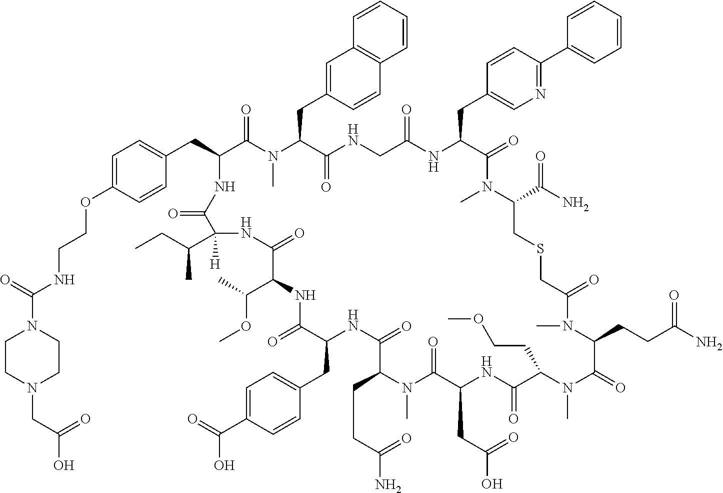 | HPLC-MS (Method A-2): 3.74 min ESI-MS m/z: 996.8 [M + 2H]2+ |
| 2-20 | 38 | 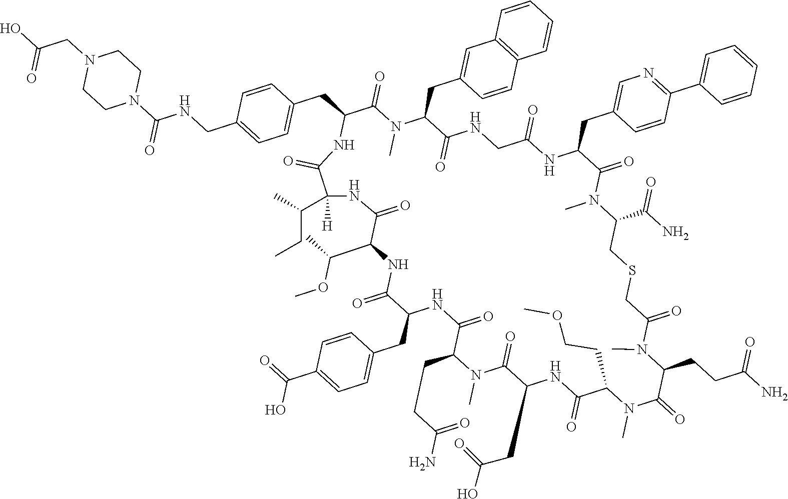 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCC(N)=O)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(CNC(=O)N3CCN(CC(=O)O)CC3)cc2)NC1=O] | HPLC-MS (Method A-2): 3,98 min ESI-MS m/z: 981.4 [M + 2H]2+ |
| 2-21 | 39 | 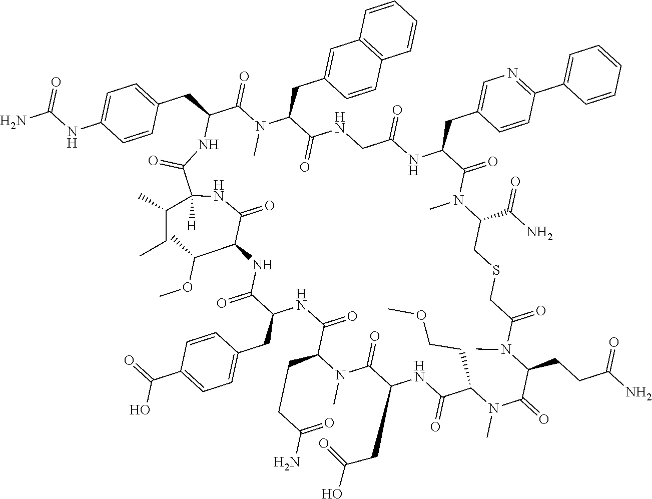 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCC(N)=O)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(NC(N)=O)cc2)NC1=O] | HPLC-MS (Method A-2): 4.50 min ESI-MS m/z: 910.8 [M + 2H]2+ |
| 2-22 | 40 | 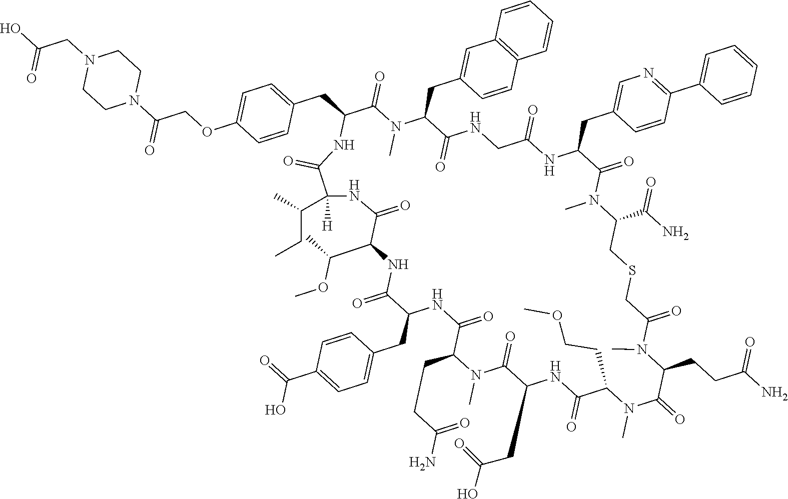 | HPLC-MS (Method A-2): 3.66 min ESI-MS m/z: 982.3 [M + 2H]2+ |
| 2-23 | 43 | 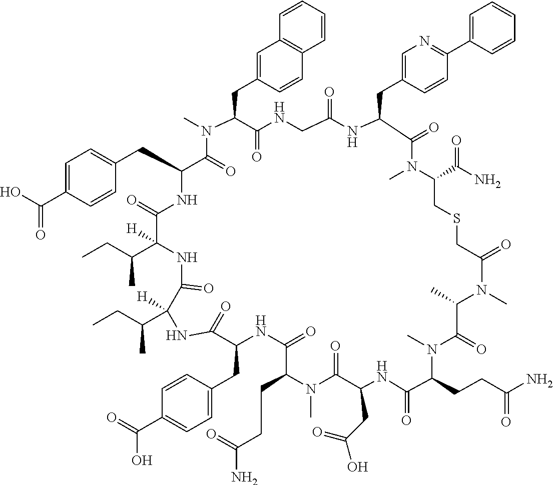 | HPLC-MS (Method A-2): 4.55 min ESI-MS m/z: 881.3 [M + 2H]2+ |
| 2-24 | 58 | 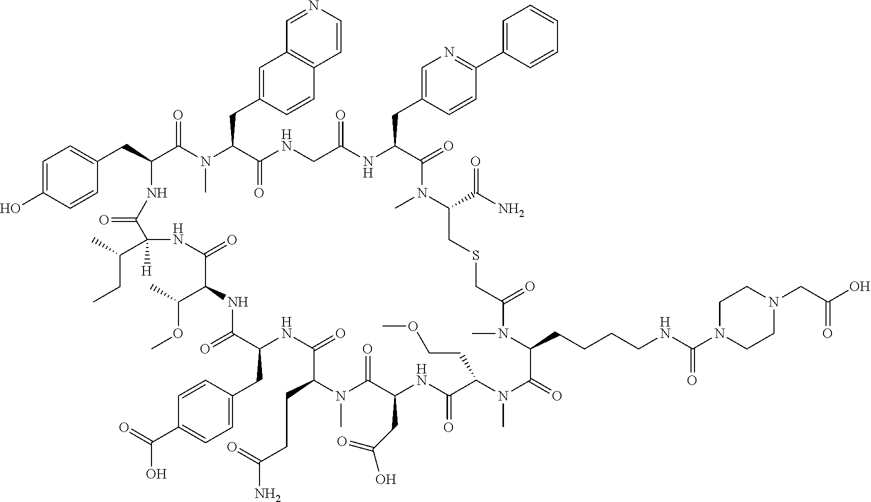 | HPLC-MS (Method A-1): 4.98 min ESI-MS m/z: 650.8 [M + 3H]3+ |
| 2-25 | 59 | 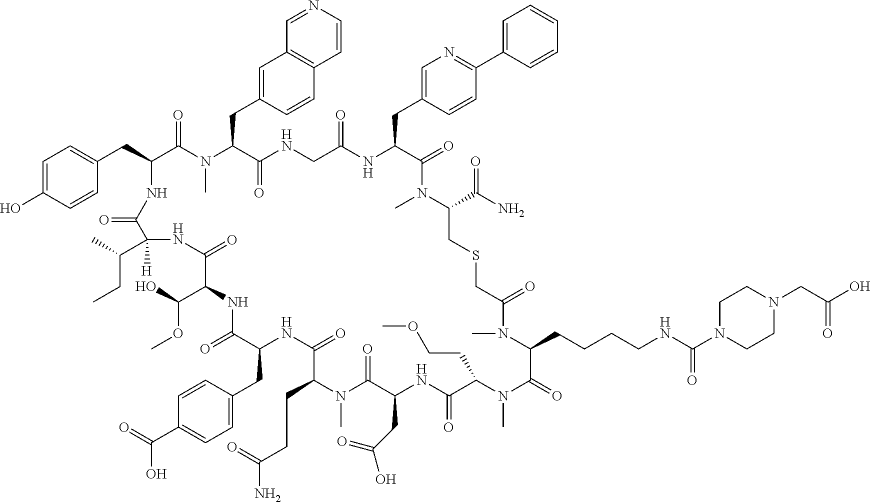 [SMILES: CC[C@H](C)[C@@H]1NC(=O)[C@H]([C@H](O)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCNC(=O)N2CCN(CC(=O)O)CC2)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC1=O] | HPLC-MS (Method A-1): 4.89 min ESI-MS m/z: 975.5 [M + 2H]2+ |
| 2-26 | 30 | 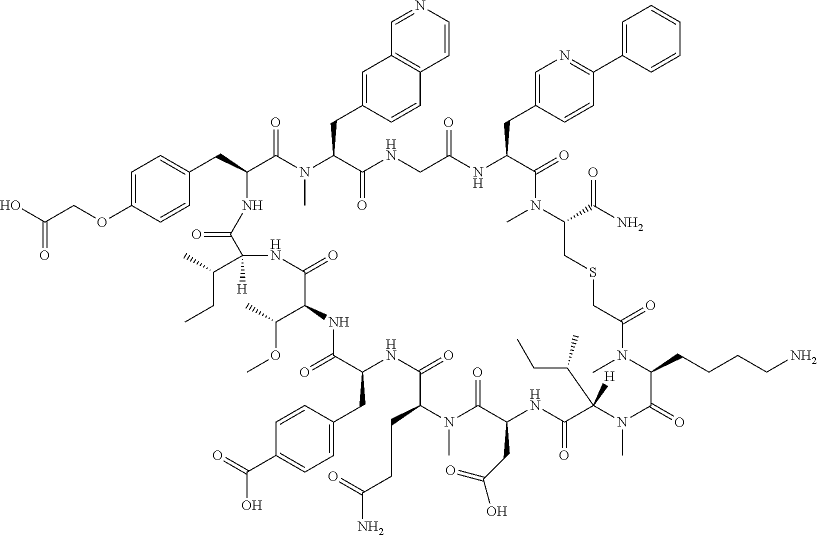 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(OCC(=O)O)cc2)NC1=O] | HPLC-MS (Method A-1): 5.50 min ESI-MS m/z: 918.4 [M + 2H]2+ |
| 2-27 | 35 | 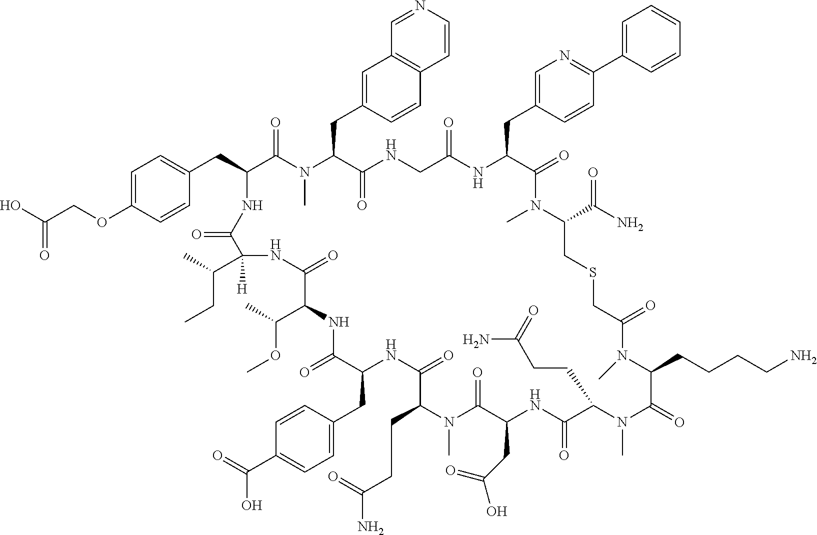 | HPLC-MS (Method A-1): 4.98 min ESI-MS m/z: 617.6 [M + 3H]3+ |
| 2-28 | 44 | 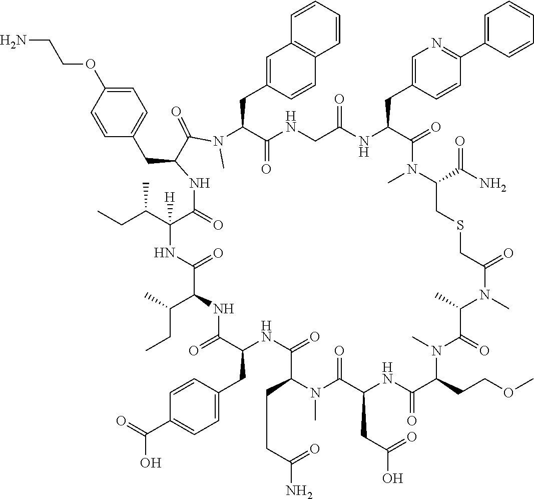 | HPLC-MS (Method A-2): 4.28 min ESI-MS m/z: 882.9 [M + 2H]2+ |
| 2-29 | 46 | 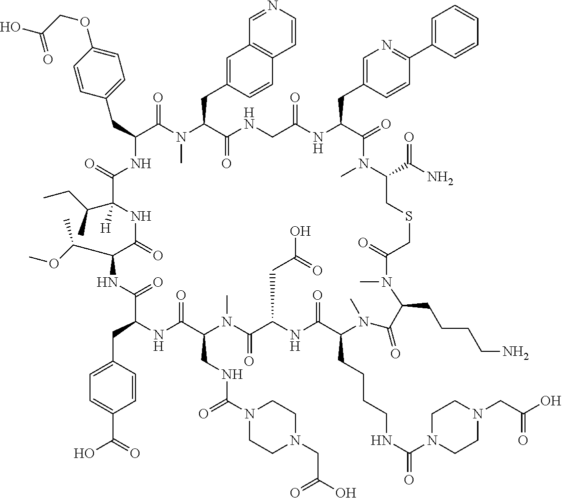 | HPLC-MS (Method A-1): 4.50 min ESI-MS m/z: 717.0 [M + 3H]3+ |
| 2-30 | 47 | 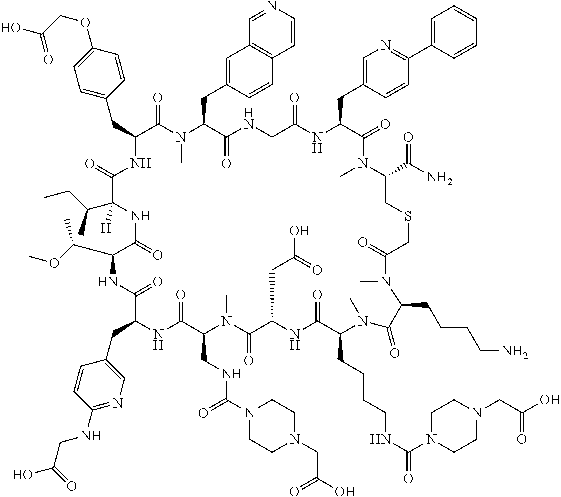 | HPLC-MS (Method A-1): 3.98 min ESI-MS m/z: 727.1 [M + 3H]3+ |
| 2-31 | 48 | 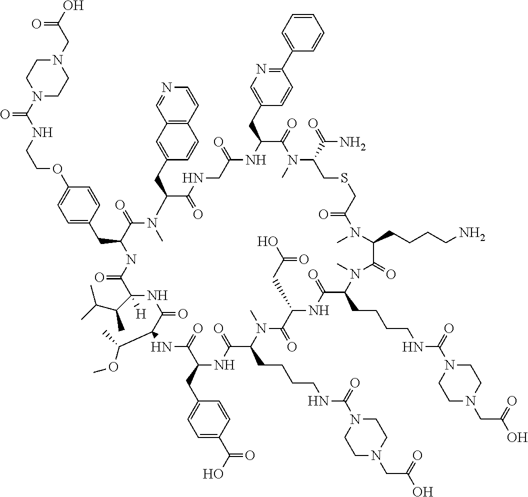 | HPLC-MS (Method A-1): 4.27 min ESI-MS m/z: 782.8 [M + 3H]3+ |
| 2-32 | 49 | 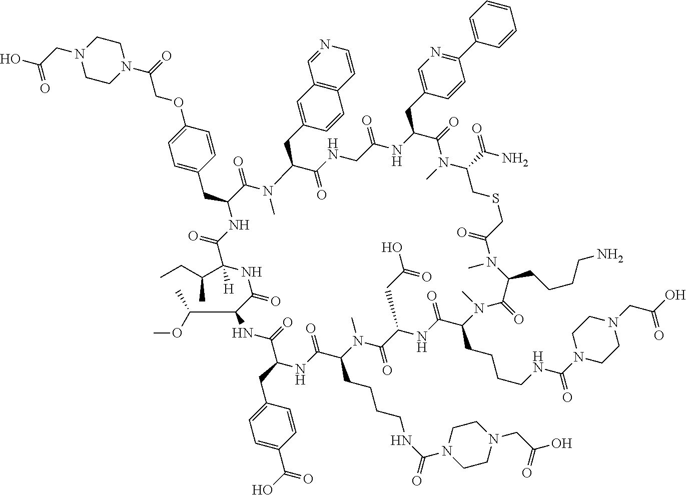 | HPLC-MS (Method A-1): 4.23 min ESI-MS m/z: 773.1 [M + 3H]3+ |
| 2-33 | 50 | 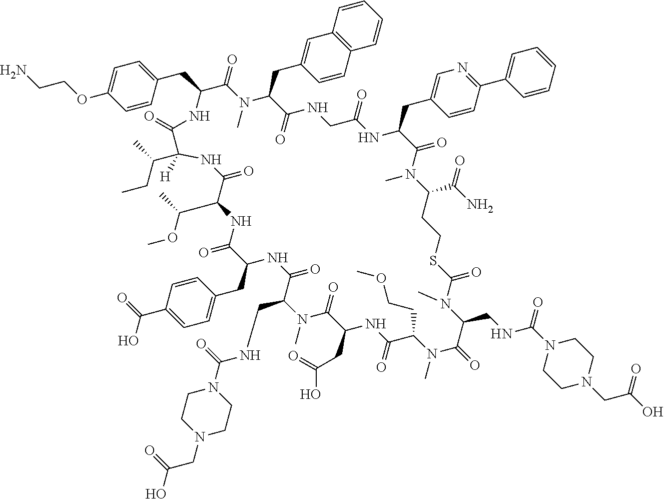 | HPLC-MS (Method A-2): 3.30 min ESI-MS m/z: 693.4 [M + 3H]3+ |
| 2-34 | 51 | 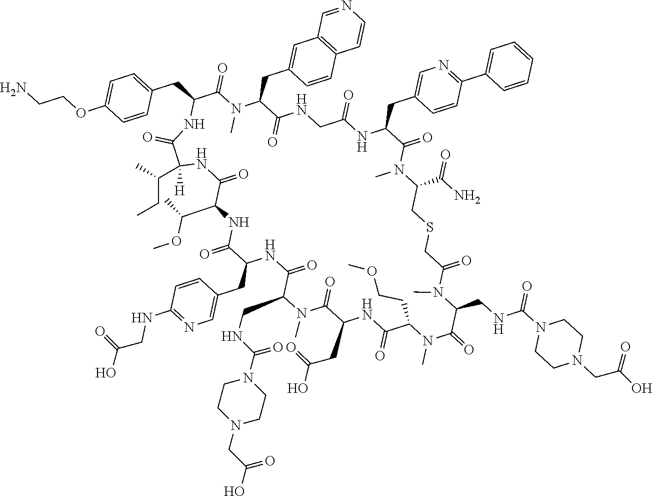 | HPLC-MS (Method A-1): 3.66 min ESI-MS m/z: 703.7 [M + 3H]3+ |
| 2-35 | 52 | 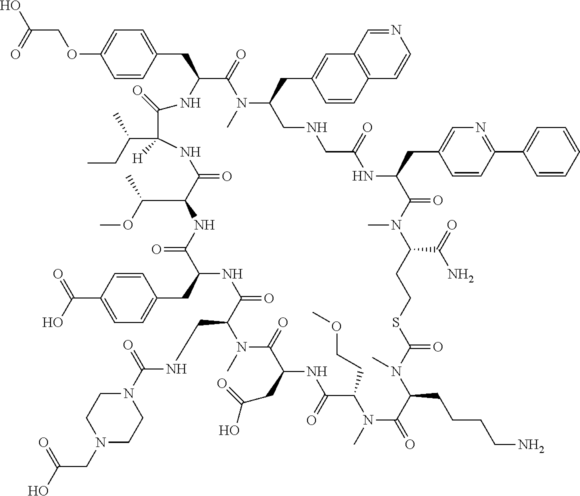 | HPLC-MS (Method A-1): 4.97 min ESI-MS m/z: 656.0 [M + 3H]3+ |
| 2-36 | 53 | 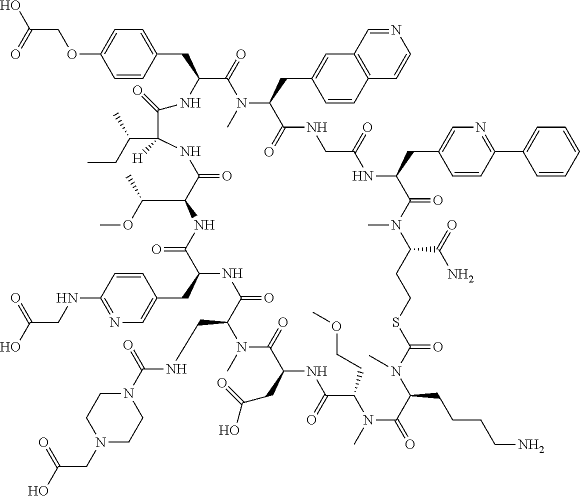 | HPLC-MS (Method A-1): 4.28 min ESI-MS m/z: 666.1 [M + 3H]3+ |
| 2-37 | 54 | 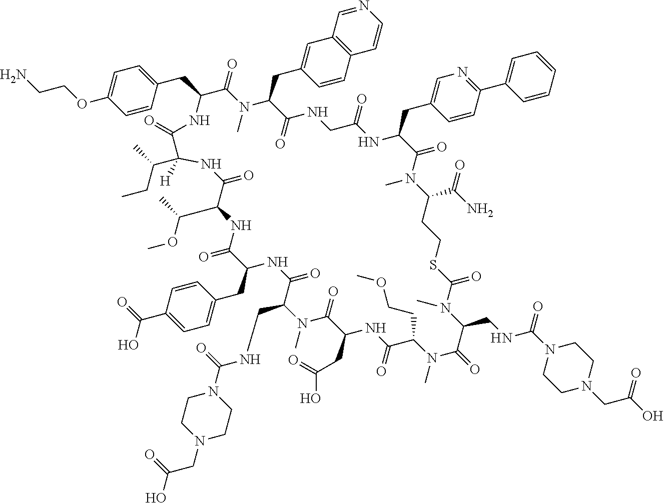 | HPLC-MS (Method A-1): 4.52 min ESI-MS m/z: 693.7 [M + 3H]3+ |
| 2-38 | 55 | 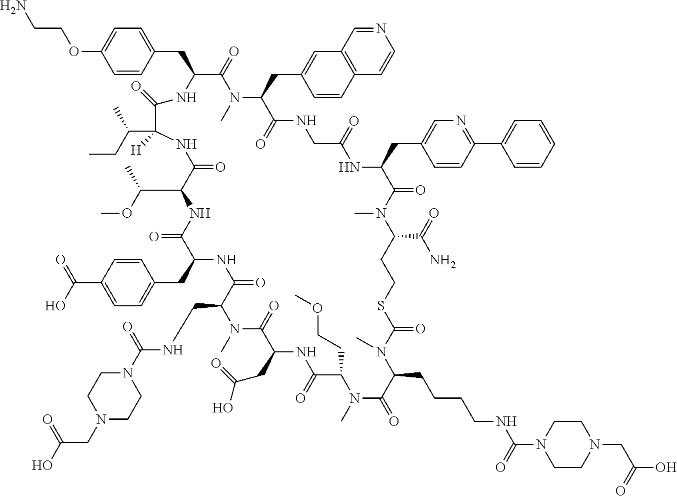 | HPLC-MS (Method A-1): 4.58 min ESI-MS m/z: 707.7 [M + 3H]3+ |
| 2-39 | 67 | 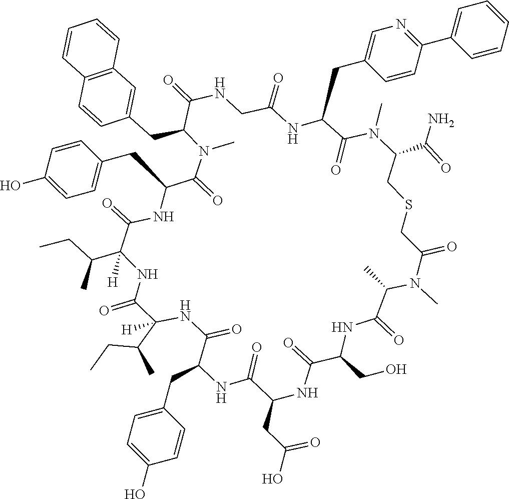 [SMILES: CC[C@H](C)[C@@H]1NC(=O)[C@H](Cc2ccc(O)cc2)NC(=O)[C@H](CC(=O)O)NC(=O)[C@H](CO)NC(=O)[C@H](C)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(O)cc2)NC(=O)[C@H]([C@@H](C)CC)NC1=O] | HPLC-MS (Method A-2): 4.80 min ESI-MS m/z: 754.4 [M + 2H]2+ |
| 2-40 | 66 | 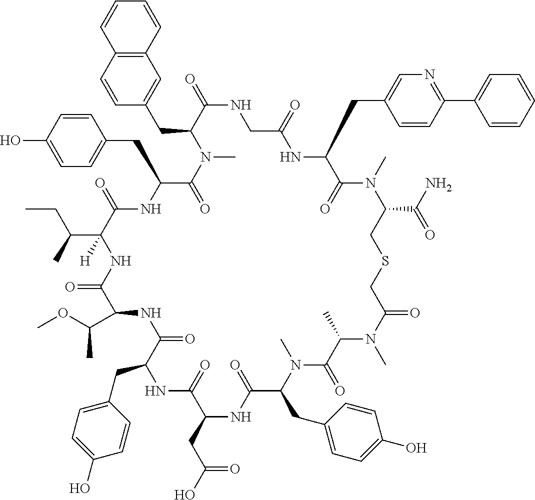 [SMILES: CC[C@H](C)[C@@H]1NC(=O)[C@H]([C@@H](C)OC)NC(=O)[C@H](Cc2ccc(O)cc2)NC(=O)[C@H](CC(=O)O)NC(=O)[C@H](Cc2ccc(O)cc2)N(C)C(=O)[C@H](C)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(O)cc2)NC1=O] | HPLC-MS (Method A-2): 4.95 min ESI-MS m/z: 800.3 [M + 2H]2+ |
| 2-41 | 68 | 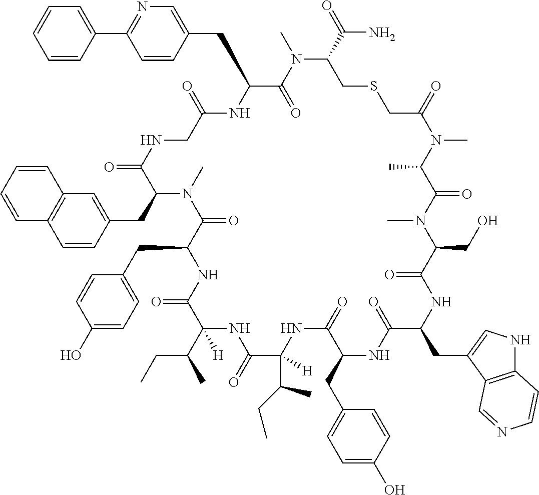 | HPLC-MS (Method A-2): 4.09 min ESI-MS m/z: 790.4 [M + 2H]2+ |
| 2-42 | 31 | 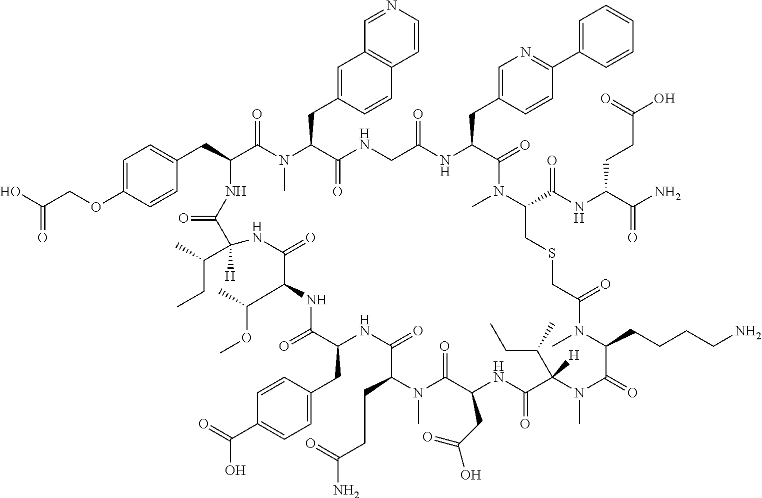 | HPLC-MS (Method A-1): 5.46 min ESI-MS m/z: 655.6 [M + 3H]3+ |
| 2-43 | 32 | 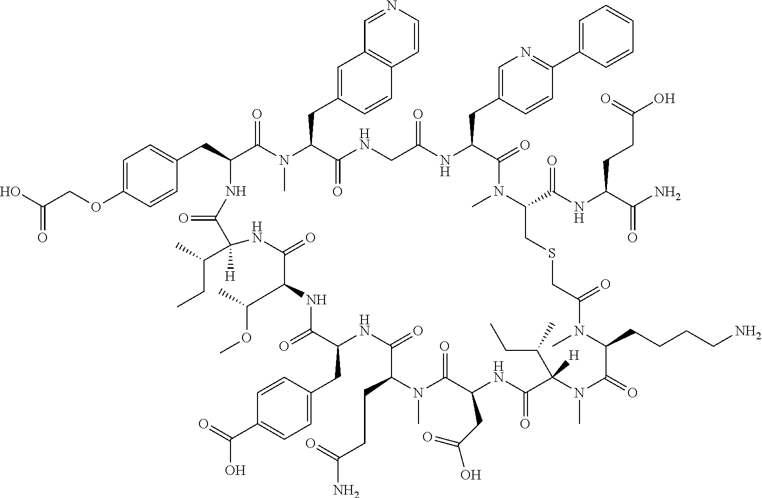 | HPLC-MS (Method A-1): 5.42 min ESI-MS m/z: 655.6 [M + 3H]3+ |
| 2-44 | 69 | 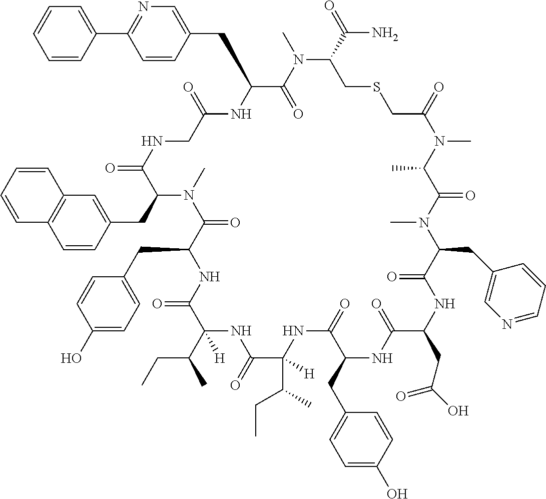 [SMILES: CC[C@@H](C)[C@@H]1NC(=O)[C@H](Cc2ccc(O)cc2)NC(=O)[C@H](CC(=O)O)NC(=O)[C@H](Cc2cccnc2)N(C)C(=O)[C@H](C)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(O)cc2)NC(=O)[C@H]([C@@H](C)CC)NC1=O] | HPLC-MS (Method A-2): 4.39 min ESI-MS m/z: 791.8 [M + 2H]2+ |
| 2-45 | 5 | 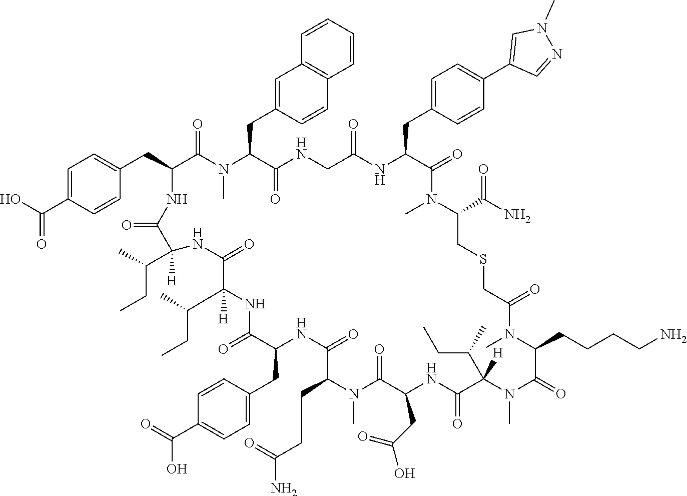 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCN)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 4.93 min ESI-MS m/z: 903.5 [M + 2H]2+ |
| 2-46 | 6 | 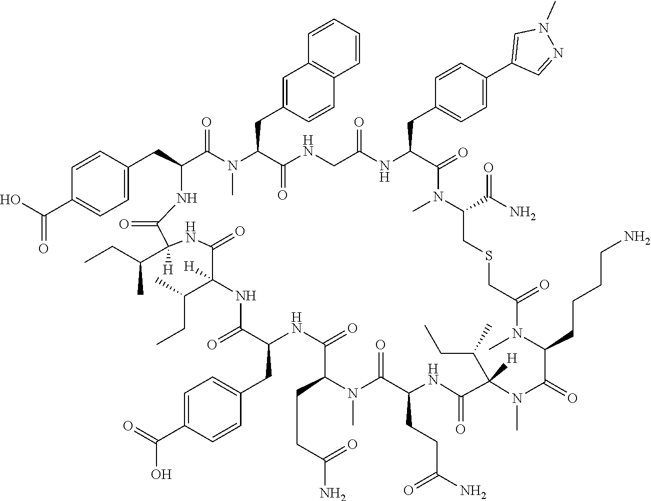 [SMILES: CC[C@H](C)[C@@H]1NC(=O)[C@H](Cc2ccc(C(=O)O)cc2)NC(=O)[C@H](CCC(N)=O)N(C)C(=O)[C@H](CCC(N)=O)NC(=O)[C@H]([C@@H](C)CC)N(C)C(=O)[C@H](CCCCN)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(C(=O)O)cc2)NC(=O)[C@H]([C@@H](C)CC)NC1=O] | HPLC-MS (Method A-2): 4.86 min ESI-MS m/z: 910.0 [M + 2H]2+ |
| 2-47 | 21 | 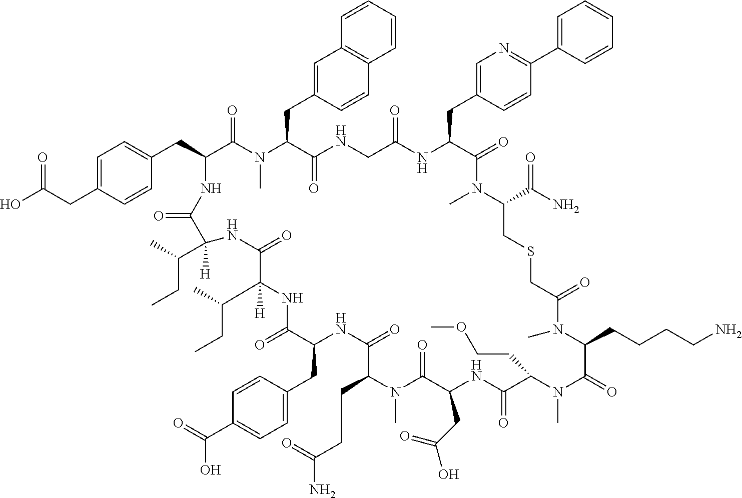 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(CC(=O)O)cc2)NC(=O)[C@H]([C@@H](C)CC)NC1=O] | HPLC-MS (Method A-2): 4.52 min ESI-MS m/z: 910.0 [M + 2H]2+ |
| 2-48 | 22 | 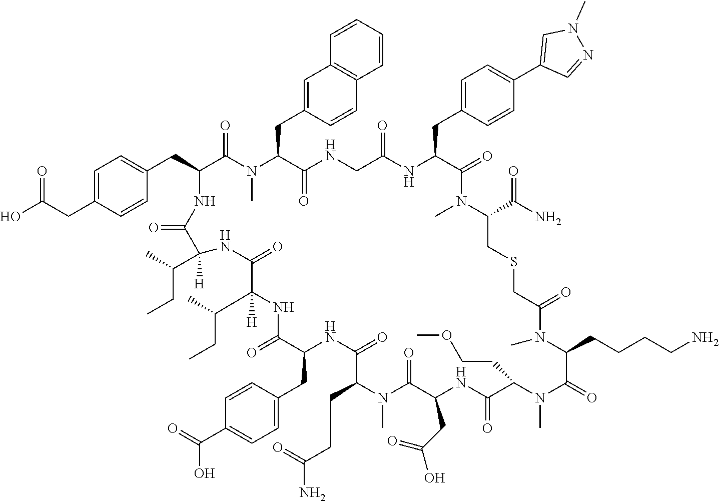 | HPLC-MS (Method A-2): 4.58 min ESI-MS m/z: 911.8 [M + 2H]2+ |
| 2-49 | 23 | 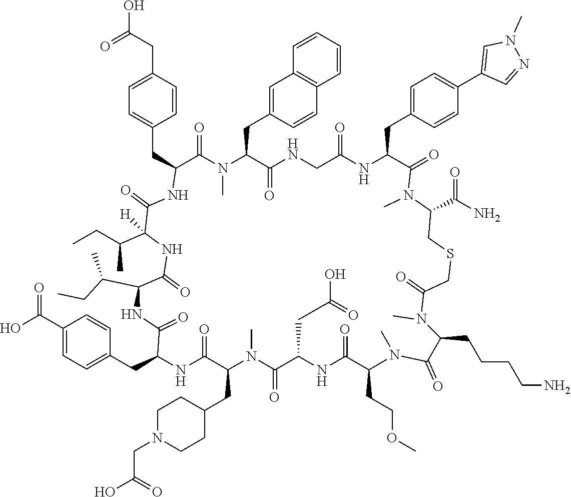 | HPLC-MS (Method A-2): 4.15 min ESI-MS m/z: 953.9 [M + 2H]2+ |
| 2-50 | 24 | 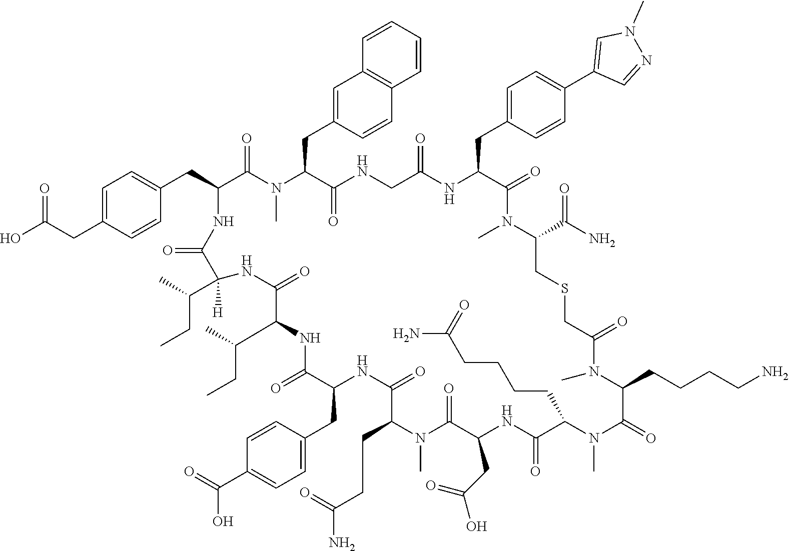 | HPLC-MS (Method A-2): 4.19 min ESI-MS m/z: 919.3 [M + 2H]2+ |
| 2-51 | 25 | 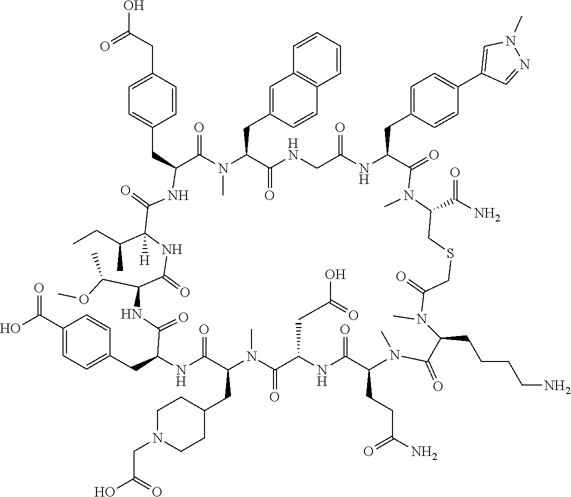 | HPLC-MS (Method A-2): 3.98 min ESI-MS m/z: 961.4 [M + 2H]2+ |
| 2-52 | 26 | 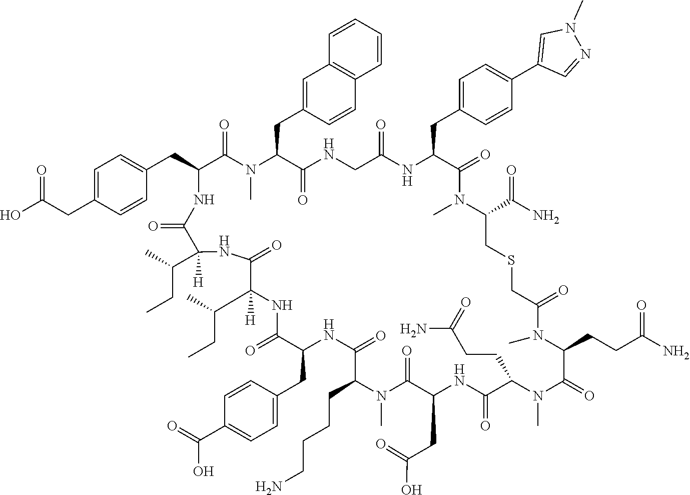 [SMILES: CC[C@H](C)[C@@H]1NC(=O)[C@H](Cc2ccc(C(=O)O)cc2)NC(=O)[C@H](CCCCN)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(CC(=O)O)cc2)NC(=O)[C@H]([C@@H](C)CC)NC1=O] | HPLC-MS (Method A-2): 4.81 min ESI-MS m/z: 917.9 [M + 2H]2+ |
| 2-53 | 28 | 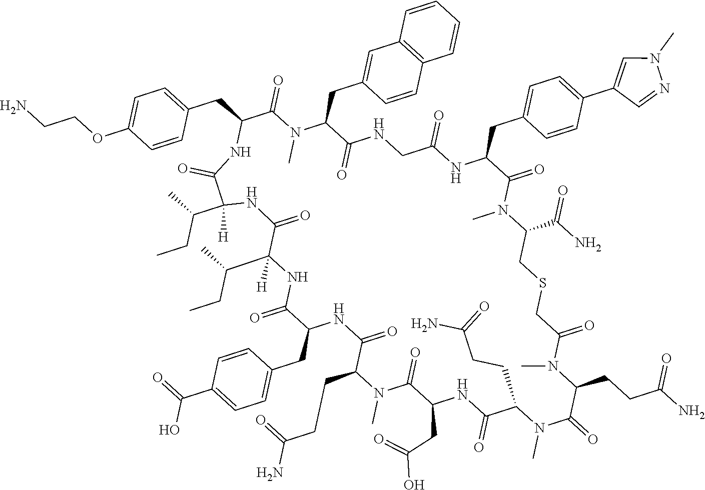 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(OCCN)cc2)NC(=O)[C@H]([C@@H](C)CC)NC1=O] | HPLC-MS (Method A-2): 4.17 min ESI-MS m/z: 918.4 [M + 2H]2+ |
| 2-54 | 29 | 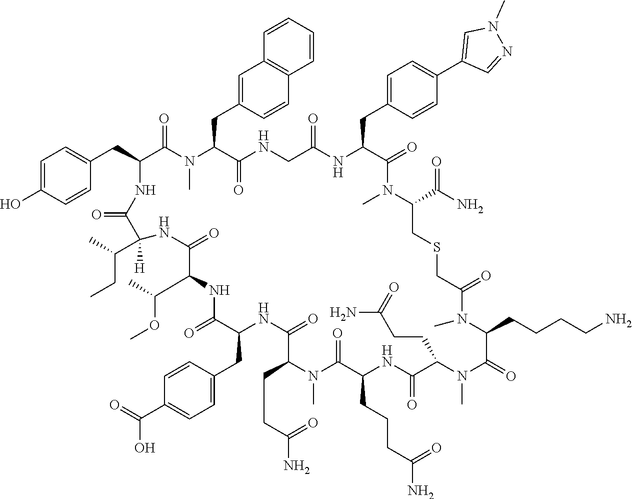 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CCCC(N)=O)NC(=O)[C@H](CCC(N)=O)N(C)C(=O)[C@H](CCCCN)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(O)cc2)NC1=O] | HPLC-MS (Method A-2): 4.30 min ESI-MS m/z: 911.4 [M + 2H]2+ |
| 2-55 | 56 | 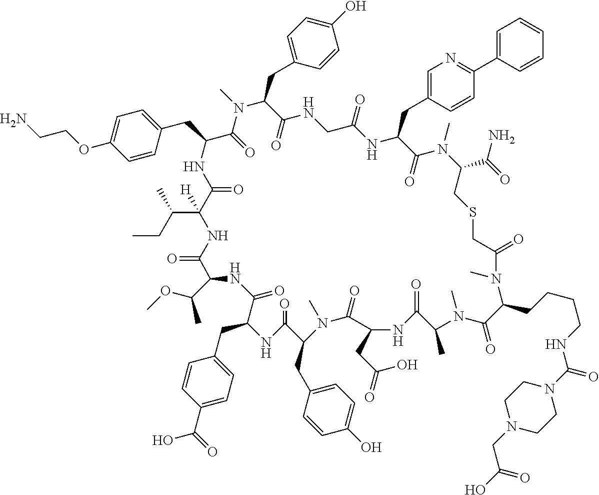 [SMILES: CC[C@H](C)[C@@H]1NC(=O)[C@H]([C@@H](C)OC)NC(=O)[C@H](Cc2ccc(C(=O)O)cc2)NC(=O)[C@H](Cc2ccc(O)cc2)N(C)C(=O)[C@H](CC(=O)O)NC(=O)[C@H](C)N(C)C(=O)[C@H](CCCCNC(=O)N2CCN(CC(=O)O)CC2)N(C)C(=O)CSC[C@@H](C(N)=O)N(C)C(=O)[C@H](Cc2ccc(-c3ccccc3)nc2)NC(=O)CNC(=O)[C@H](Cc2ccc(O)cc2)N(C)C(=O)[C@H](Cc2ccc(OCCN)cc2)NC1=O] | HPLC-MS (Method A-1): 5.20 min ESI-MS m/z: 975.0 [M + 2H]2+ |
| 2-56 | 27 | 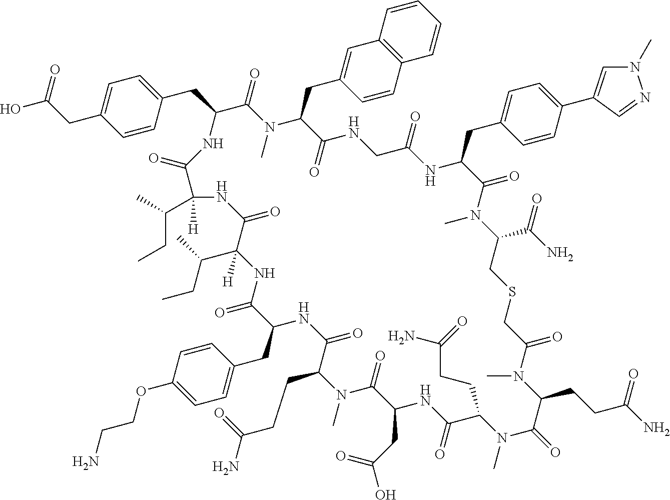 [SMILES: CC[C@H](C)[C@@H]1NC(=O)[C@H](Cc2ccc(OCCN)cc2)NC(=O)[C@H](CCC(N)=O)N(C)C(=O)[C@H](CC(=O)O)NC(=O)[C@H](CCC(N)=O)N(C)C(=O)[C@H](CCC(N)=O)N(C)C(=O)CSC[C@@H](C(N)=O)N(C)C(=O)[C@H](Cc2ccc(-c3cnn(C)c3)cc2)NC(=O)CNC(=O)[C@H](Cc2ccc3ccccc3c2)N(C)C(=O)[C@H](Cc2ccc(CC(=O)O)cc2)NC(=O)[C@H]([C@@H](C)CC)NC1=O] | HPLC-MS (Method A-2): 4.39 min ESI-MS m/z: 925.5 [M + 2H]2+ |
| 2-57 | 1 | 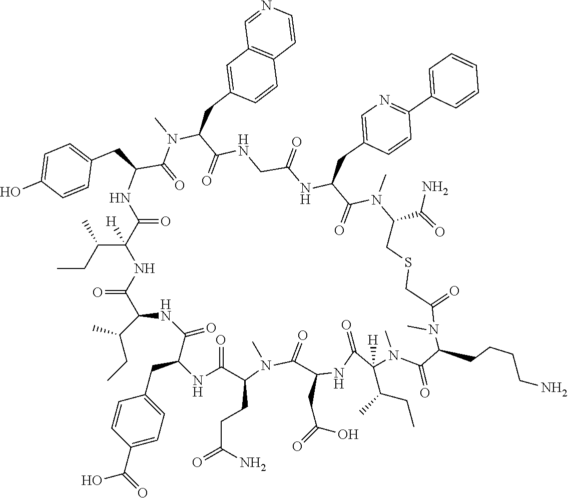 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O] | HPLC-MS (Method A-1): 5.79 min ESI-MS m/z: 888.4 [M + 2H]2+ |
| 2-58 | 61 | 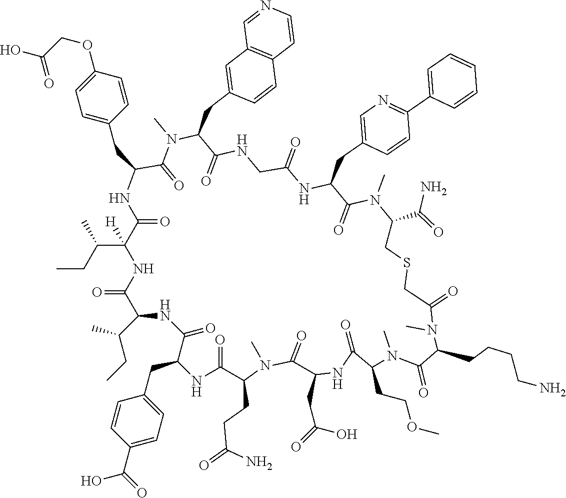 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(OCC(=O)O)cc2)NC(=O)[C@H]([C@@H](C)CC)NC1=O] | HPLC-MS (Method A-1): 5.52 min ESI-MS m/z: 918.4 [M + 2H]2+ |
| 2-59 | 63 | 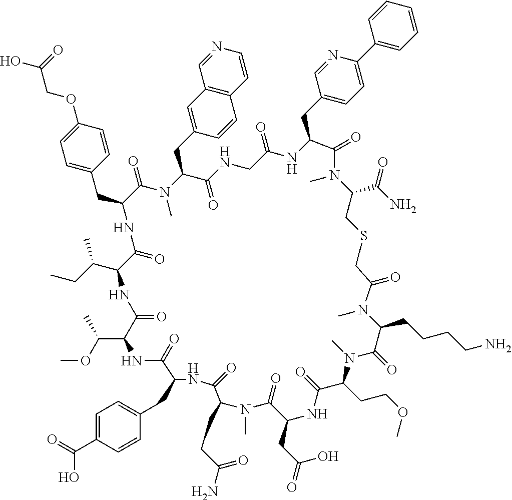 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(OCC(=O)O)cc2)NC1=O] | HPLC-MS (Method A-1): 4.96 min ESI-MS m/z: 919.4 [M + 2H]2+ |
| 2-60 | 62 | 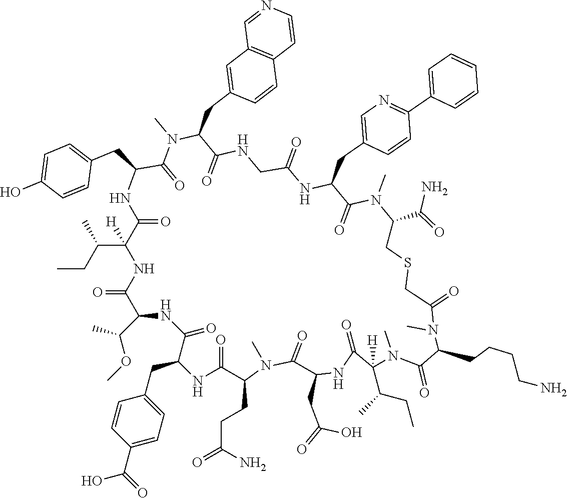 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC1=O] | HPLC-MS (Method A-1): 5.32 min ESI-MS m/z: 889.4 [M + 2H]2+ |
| 2-61 | 72 | 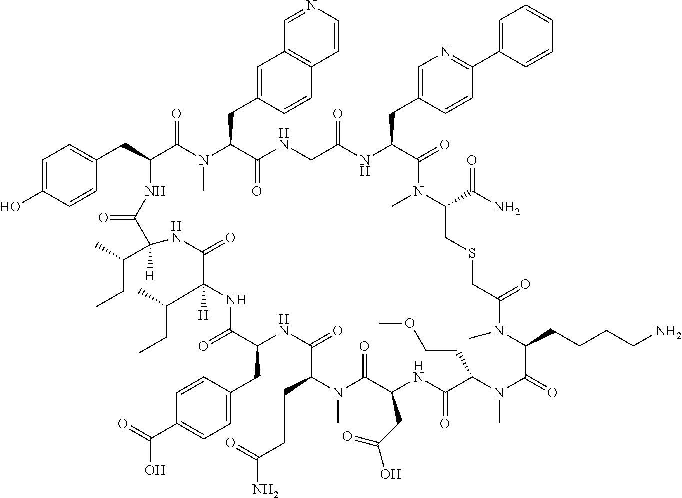 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCN)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O] | HPLC-MS (Method A-1): 4.96 min ESI-MS m/z: 889.4 [M + 2H]2+ |

### Example B3: Synthesis of the Conjugated Form Consisting of the GPC Binding Peptide in this Disclosure and Biotin as a Payload

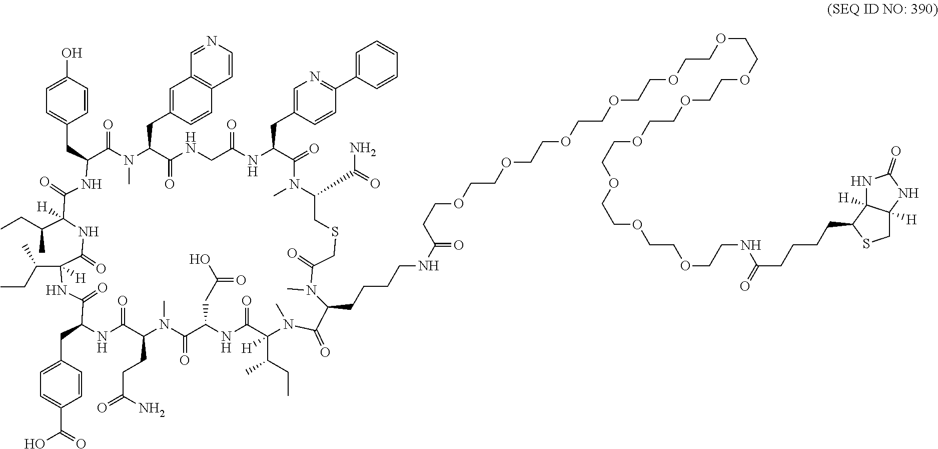 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(=O)CCOCCOCCOCCOCCOCCOCCOCCOCCOCCOCCOCCOCCNC(=O)CCCC[C@@H]2SC[C@@H]3NC(=O)N[C@@H]32)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O]

The desired polypeptide was synthesized as outlined in Scheme-P3 on Fmoc-Sieber amide resin (0.100 mmol). Elongation of the linear peptide was achieved by Method S-1 employing Fmoc-AAs listed in below table. The obtained peptide on the resin was treated with the reagent mixture (4.5 mL) and performed the chloroacetylation as described in Step-C2 at room temperature for 30 min. Subsequently, the resin was shaken with Pd(PPh₃)₄(23.1 mg, 0.2 eq) and PhSiH₃ (123 μL, 10 eq) in DCM (5 mL) at room temperature for 1 hr. The obtained peptide on the resin was shaken with Boc-PEG12c-OH (0.53 mmol) in DMF (2.5 mL)/PyAOP (0.50 mmol) and DIEA (1.0 mmol) in DMF (2 mL) at 40° C. for 30 min as outlined in step-C3. The polypeptide on the resin was treated with the cleavage cocktail (4.5 mL) for 60 min as outlined in step-C4 to furnish the linear peptide, which was dissolved in MeCN/H₂O (20 mL). To the mixture was added Et3N (279 L), and then the mixture was shaken for at room temperature for 15 hrs, and then concentrated as outlined in Step-C5. The reaction mixture was concentrated in Genevac EZ-2 Elite, and then the resulting residue was dissolved in DMSO (4 mL). To the solution were added Biotin-OSu (34.1 mg, 0.1 mmol) and DIEA (52.3 L). The mixture was shaken at room temperature for 1 hr as outlined in Step-C6. To the mixture was added AcOH. The mixture was directly purified by preparative RP-HPLC. Pure fractions were combined and lyophilized to afford the title macrocyclic peptide (Conjugate ID NO.: PLB-1). HPLC-MS (Method A-2): 4.13 min, ESI-MS m/z: 868.0 [M+3H]3+.

TABLE 11

| order of Fmoc-AA | AA position in | |
|---|---|---|
| used in the SPPS | the cyclic peptide | Fmoc-AA |
| 1 | X12 | Fmoc-MeC(Trt)-OH |
| 2 | X11 | Fmoc-3Py6Ph-OH |
| 3 | X10 | Fmoc-G-OH |
| 4 | X9 | Fmoc-MeNal27N |
| 5 | X8 | Fmoc-Y(tBu)-OH |
| 6 | X7 | Fmoc-I-OH |
| 7 | X6 | Fmoc-I-OH |
| 8 | X5 | Fmoc-F4COO(tBu)-OH |
| 9 | X4 | Fmoc-Q(Trt)-OH |
| 10 | X3 | Fmoc-D(Mpe)-OH |
| 11 | X2 | Fmoc-MeI-OH |
| 12 | XI | Fmoc-MeK(Alloc)-OH |

### Example B4: The Following Conjugates were Prepared Analogously to Example-3

TABLE 12

| | Conju- | | |
|---|---|---|---|
| | gate | | |
| | ID/ | | |
| | (SEQ | | |
| Ex. | ID | | Analytical |
| No. | NO) | Structure | data |
| 4-1 | PLB- 35/ (391) | 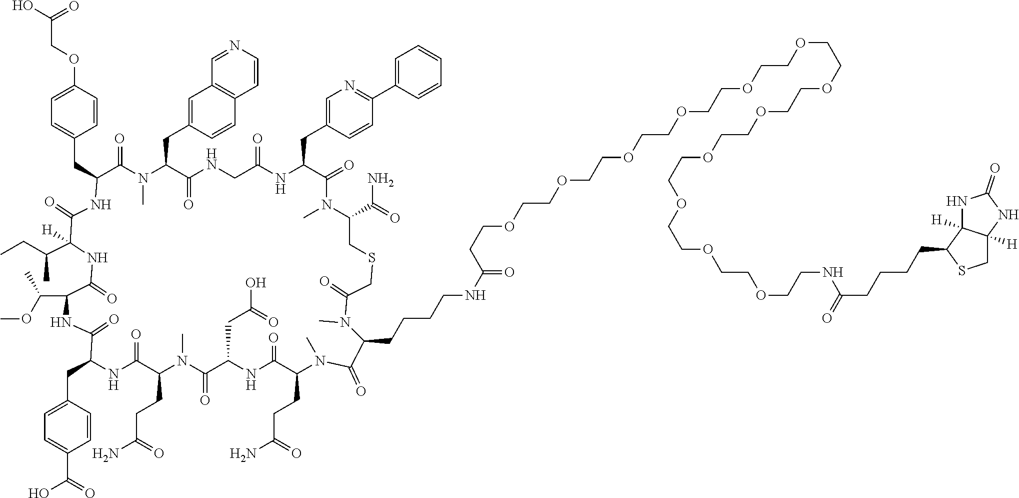 | HPLC-MS (Method A- 2): tr = 3.92 min ESI-MS m/z: 892.9 ([M + 3H]3+) |
| 4-2 | PLB- 28/ (392) | 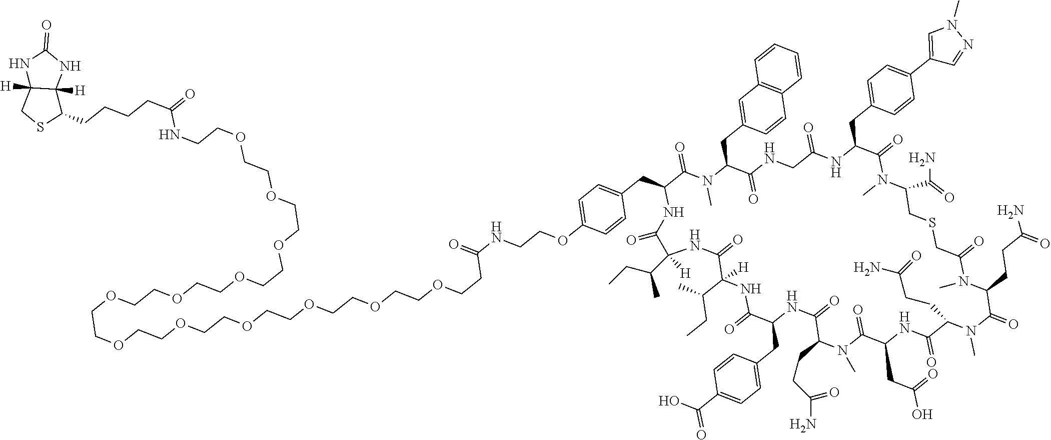 | HPLC-MS (Method A- 2): tr = 5.30 min ESI-MS m/z: 888.3 ([M + 3H]3+) |
| 4-3 | PLB- 64/ (393) | 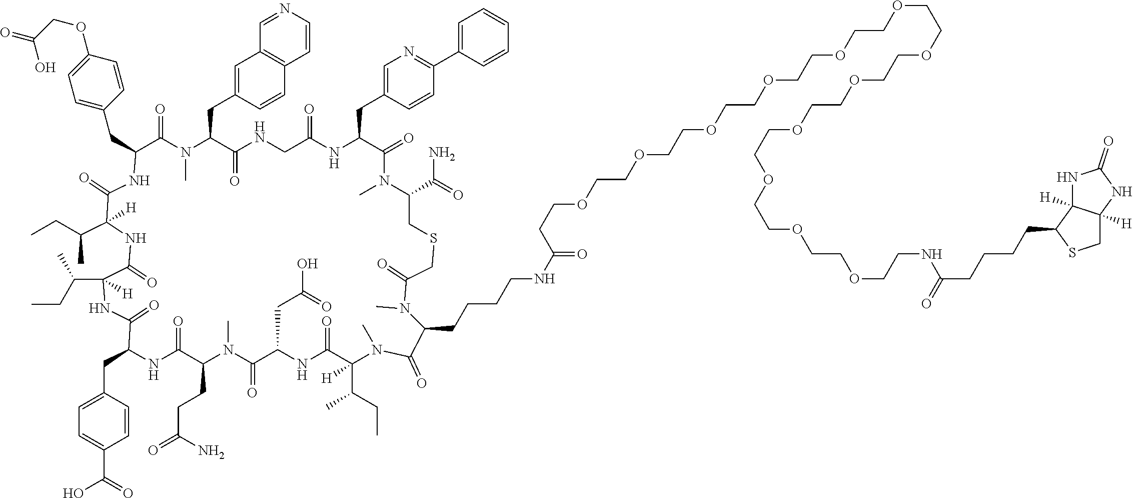 | HPLC-MS (Method A- 2): tr = 4.02 min ESI-MS m/z: 887.3 ([M + 3H]3+) |
| 4-4 | PLB- 30/ (394) | 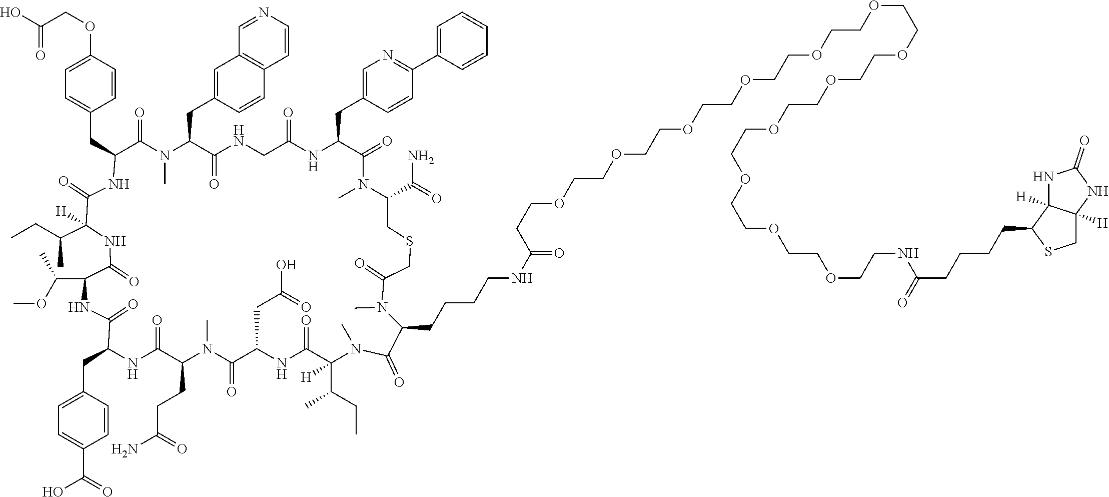 | HPLC-MS (Method A- 2) tr = 4.36 min ESI-MS m/z: 887.9 ([M + 3H]3+) |

### Example B5: Synthesis of the Conjugated Form Consisting of the GPC Binding Peptide in this Disclosure and Biotin as a Payload

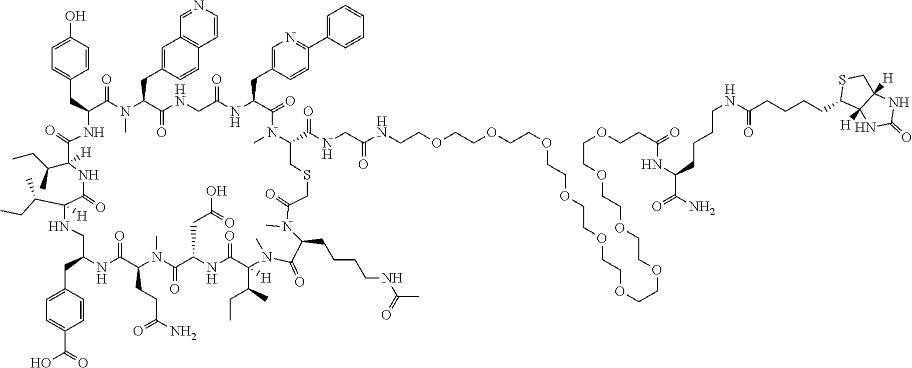 [SMILES: CC[C@H](C)[C@@H]1NC[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(C)=O)N(C)C(=O)CSC[C@@H](C(=O)NCC(=O)NCCOCCOCCOCCOCCOCCOCCOCCOCCOCCOCCC(=O)N[C@@H](CCCCNC(=O)CCCC[C@@H]2SC[C@@H]3NC(=O)N[C@@H]32)C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O]

The desired polypeptide was synthesized as outlined in Scheme-P1 on Fmoc-Sieber amide resin (0.050 mmol). Elongation of the linear peptide except for X9 was achieved by Method S-1 employing Fmoc-AAs listed in below table. Coupling reaction of Fmoc-MeNal27N-OH was performed manually as described in Method S4. The obtained peptide on the resin was treated with the reagent mixture (6 mL) and performed the chloroacetylation as described in Step-A2 at room temperature for 30 min. The polypeptide on the resin was treated with the cleavage cocktail (3 mL) for 30 min as outlined in Step-A3 to furnish the linear peptide, which was dissolved in MeCN/H₂O (10 mL). To the mixture was added Et₃N (69.7 μL), and then the mixture was shaken for at room temperature for 3 hrs, and then concentrated as outlined in Step-A4. The resulting residue was purified by preparative RP-HPLC. Pure fractions were combined and lyophilized to afford the title macrocyclic peptide (Conjugate ID No.: PLB-60). HPLC-MS (Method A-2): 3.75 min, ESI-MS in/z: 914.3 [M+31-1]3+.

TABLE 13

| Reaction | Position | |
|---|---|---|
| order | no. | Fmoc-AA |
| 1 | X15 | Fmoc-K(biotin)-OH |
| 2 | X14 | Fmoc-PEG12c-OH |
| 3 | X13 | Fmoc-G-OH |
| 4 | X12 | Fmoc-MeC(Trt)-OH |
| 5 | X11 | Fmoc-3Py6Ph-OH |
| 6 | X10 | Fmoc-G-OH |
| 7 | X9 | Fmoc-MeNal27N-OH |
| 8 | X8 | Fmoc-Y(tBu)-OH |
| 9 | X7 | Fmoc-I-OH |
| 10 | X6 | Fmoc-I-OH |
| 11 | X5 | Fmoc-F4COO(tBu)-OH |
| 12 | X4 | Fmoc-MeQ(Trt)-OH |
| 13 | X3 | Fmoc-D(Mpe)-OH |
| 14 | X2 | Fmoc-MeI-OH |
| 15 | X1 | Fmoc-MeKAc-OH |

### Example B6: Synthesis of the Conjugated Form Consisting of the GPC Binding Peptide in this Disclosure and sulfoCy5 as a Payload

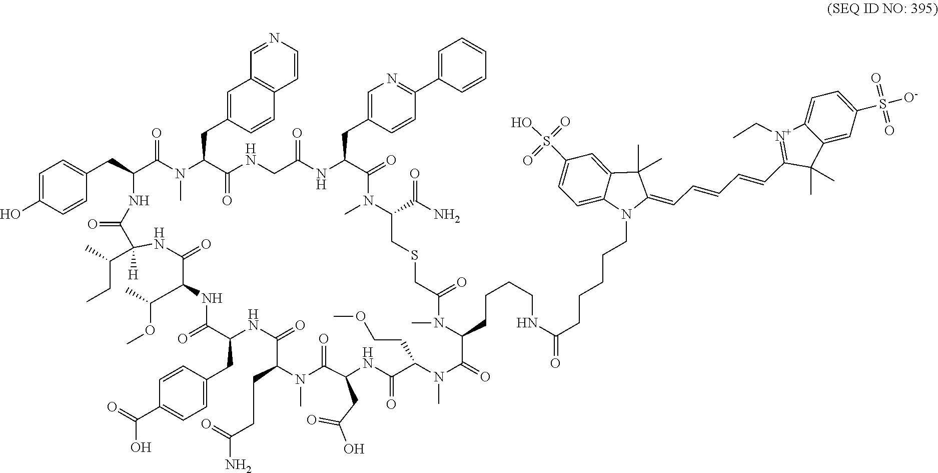 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](CCCCNC(=O)CCCCCN2/C(=C/C=C/C=C/C3=[N+](CC)c4ccc(S(=O)(=O)[O-])cc4C3(C)C)C(C)(C)c3cc(S(=O)(=O)O)ccc32)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC1=O]

The desired polypeptide was synthesized as outlined in Scheme-P4. To a solution of the GPC binding peptide in Example B1 (10.0 mg, 4.71 μmol) in DMF (150 μL) at room temperature were added triethylarsine (6.6 μL, 47.1 μmol) and SulfoCy5-NHS (4.6 mg, 6.13 μmol). The mixture was stirred at room temperature for 18 hrs as outlined in step-D4. The resulting mixture was purified by preparative RP-HPLC. Pure fractions were combined and lyophilized to afford the title macrocyclic peptide (Conjugate ID No.: PLD-45). HPLC-MS (Method A-2): 3.80 min, ESI-MS m/z: 1209.6 [M+2H]2+.

### Example B7: The Following Compounds were Prepared by Analogous Method Described Above

TABLE 14

| | Conju- | | |
|---|---|---|---|
| | gate | | |
| | ID:/ | | |
| | (SEQ | | |
| Ex. | ID | | Analytical |
| No. | NO) | Structure | data |
| 7-1 | PLD- 30/ (396) | 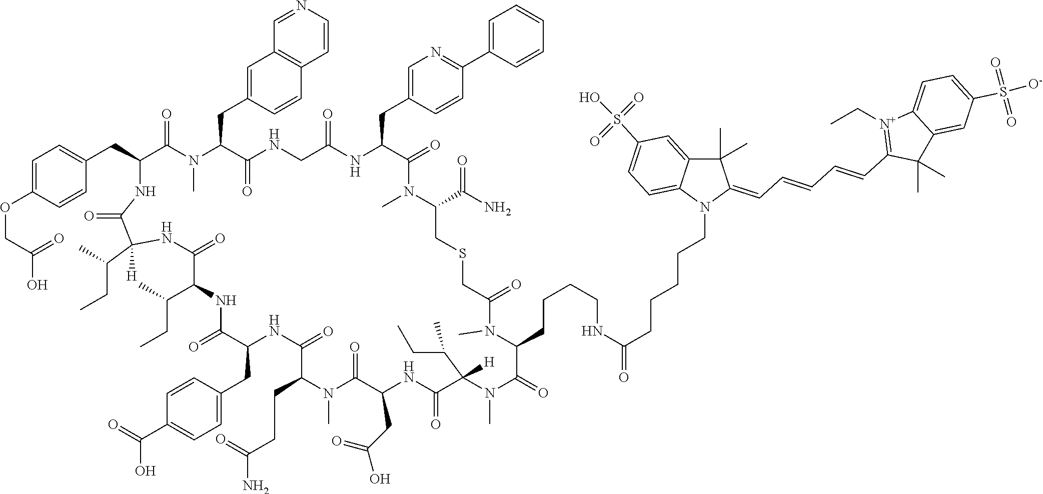 | HPLC-MS (Method A- 2): tr = 4.00 min ESI-MS m/z: 1237.7 ([M + 2H]2+) |
| 7-2 | PLD- 1/ (397) | 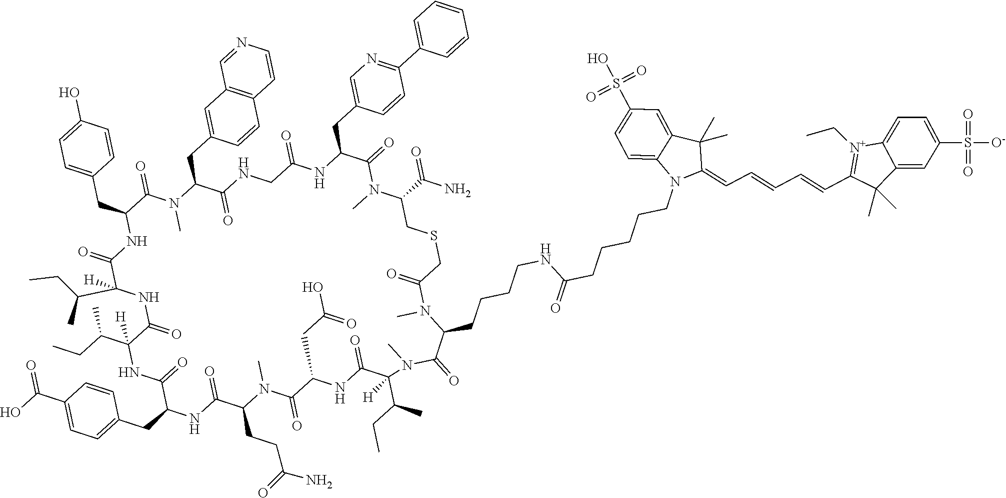 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(=O)CCCCCN2/C(=C/C=C/C=C/C3=[N+](CC)c4ccc(S(=O)(=O)[O-])cc4C3(C)C)C(C)(C)c3cc(S(=O)(=O)O)ccc32)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(O)cc2)NC(=O)[C@H]([C@@H](C)CC)NC1=O] | HPLC-MS (Method A- 2): tr = 4.14 min ESI-MS m/z: 1207.7 ([M + 2H]2+) |
| 7-3 | Mod3 (402) | 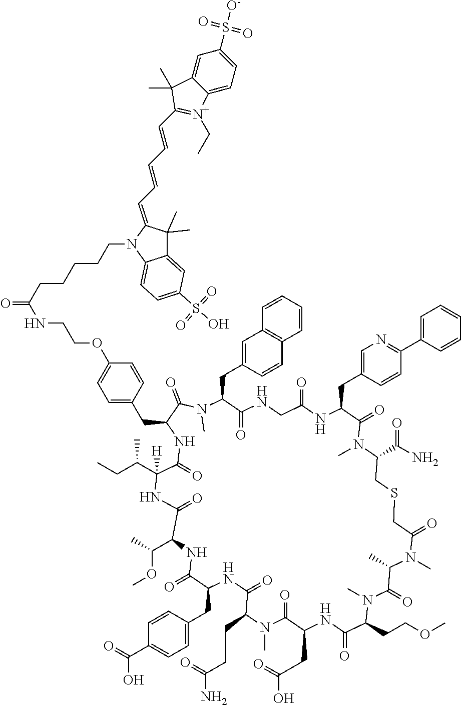 [SMILES: CC[C@H](C)[C@@H]1NC(=O)[C@H]([C@@H](C)OC)NC(=O)[C@H](Cc2ccc(C(=O)O)cc2)NC(=O)[C@H](CCC(N)=O)N(C)C(=O)[C@H](CC(=O)O)NC(=O)[C@H](CCOC)N(C)C(=O)[C@H](C)N(C)C(=O)CSC[C@@H](C(N)=O)N(C)C(=O)[C@H](Cc2ccc(-c3ccccc3)nc2)NC(=O)CNC(=O)[C@H](Cc2ccc3ccccc3c2)N(C)C(=O)[C@H](Cc2ccc(OCCNC(=O)CCCCCN3/C(=C/C=C/C=C/C4=[N+](CC)c5ccc(S(=O)(=O)[O-])cc5C4(C)C)C(C)(C)c4cc(S(=O)(=O)O)ccc43)cc2)NC1=O] | HPLC-MS (Method A- 2): tr = 5.30 min ESI-MS m/z: 1202.2 [M + 2H]2+ |
| 7-4 | Mod5 (403) | 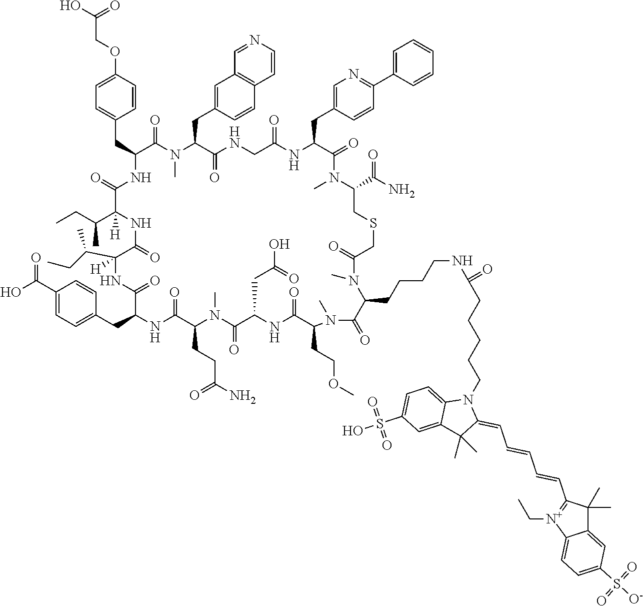 | HPLC-MS (Method A- 2): tr = 4.00 min ESI-MS m/z: 1237.8 [M + 2H]2+ |
| 7-5 | Mod6 (404) | 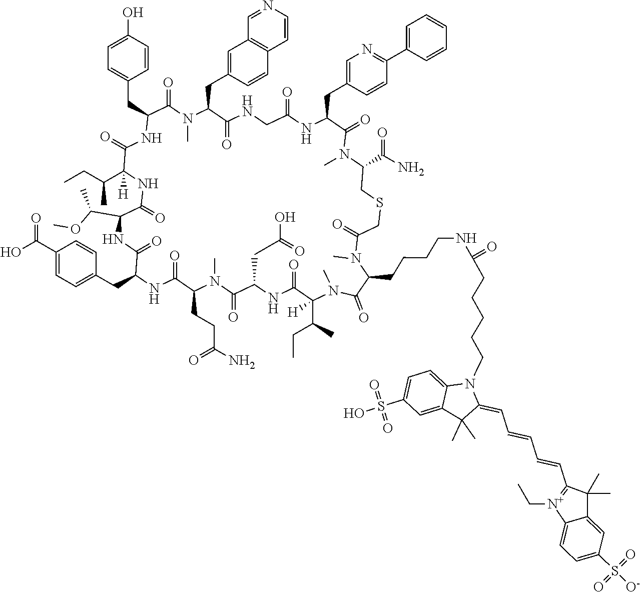 | HPLC-MS (Method A- 2): tr = 4.41 min ESI-MS m/z: 1208.8 [M + 2H]2+ |
| 7-6 | Mod7 (405) | 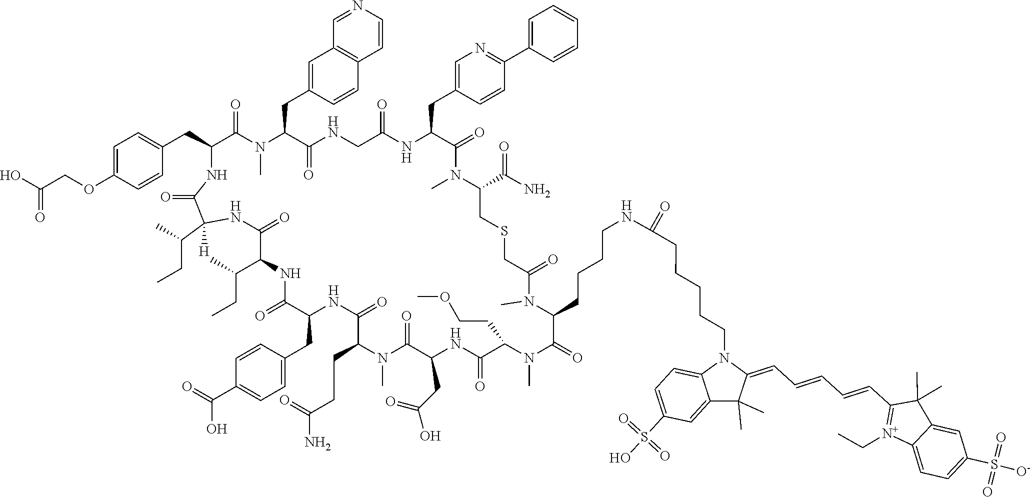 | HPLC-MS (Method A- 2): tr = 4.10 min ESI-MS m/z: 1238.8 [M + 2H]2+ |

### Example B8: The Following Compounds were Prepared by Analogous Method Described Above

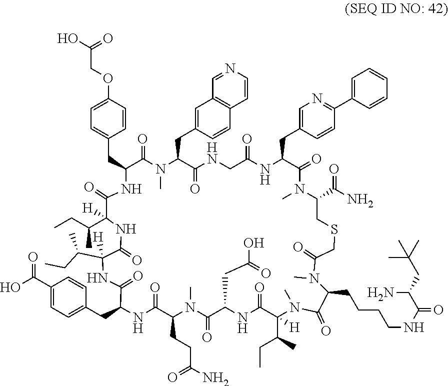 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(=O)[C@H](N)CC(C)(C)C)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(OCC(=O)O)cc2)NC(=O)[C@H]([C@@H](C)CC)NC1=O]

The desired macrocyclic peptide was synthesized as outlined in Scheme-P6. Starting with Fmoc-Sieber amide resin (2×0.250 mmol), elongation of the linear peptide except for X9 was achieved by Method S-1 employing Fmoc-AAs listed in below table. Coupling reaction of Fmoc-MeNal27N-OH for X9 was performed manually as described in Method S4. The obtained peptide on the resin was treated with Fmoc-datb-OH (0.75 mmol)/HATU (0.75 mmol)/DIEA (1.5 mmol) in DMF (10 mL) at room temperature for 30 min and then washed with DMF. Subsequently, the resin was shaken with Pd(PPh₃)₄ (57.8 mg, 0.2 eq) and PhSiH₃ (308 μL, 10 eq) in DCM/AcOH (99/1, 5 mL) at room temperature for 1 hr, and then washed successively with DCM and DMF. The resin was treated with the reagent mixture (ClAcOH (1.25 mmol)/HATU (1.25 mmol)/DIEA (2.5 mmol) in DMF (10 mL). The whole mixture was shaken at room temperature for 30 min. The polypeptide on resin was successively washed with DMF, DCM, and then Et₂O. The polypeptide on resin was dried under reducing pressure. The polypeptide on the resin was treated with the cleavage cocktail (10 mL) for 90 min as outlined in Step-A3 to furnish the linear peptide, which was dissolved in DMSO/MeCN/H₂O (1/1/1,100 mL). The mixture was split into four portions, and each mixture was then added TEA in each mixture (4×174 μL for the whole crude). The mixture was shaken at room temperature for 2 hrs, and then concentrated in Genevac EZ-2 Elite. The resulting residue was dissolved in DMSO (15 mL). To the solution was added pyrrolidine (310 μL), and then the solution was shaken at room temperature for 1 hr. The reaction mixture was rendered acidic by AcOH, and then directly purified by preparative RP-HPLC. Pure fractions were combined and lyophilized to afford the title macrocyclic peptide. HPLC-MS (Method A-2): 3.67 min, ESI-MS m/z: 654.4 [M+3H]3+.

TABLE 15

| order of Fmoc-AA | AA position in | |
|---|---|---|
| used in the SPPS | the cyclic peptide | Fmoc-AA |
| 1 | X12 | Fmoc-MeC(Trt)-OH |
| 2 | X11 | Fmoc-3Py6Ph-OH |
| 3 | X10 | Fmoc-G-OH |
| 4 | X9 | Fmoc-MeNal27N-OH |
| 5 | X8 | Fmoc-F4aao(tBu)-OH |
| 6 | X7 | Fmoc-I-OH |
| 7 | X6 | Fmoc-I-OH |
| 8 | X5 | Fmoc-F4COO(tBu)-OH |
| 9 | X4 | Fmoc-MeQ(Trt)-OH |
| 10 | X3 | Fmoc-D(Mpe)-OH |
| 11 | X2 | Fmoc-MeI-OH |
| 12 | X1 | Alloc-MeK(Fmoc)-OH |

### Example B9: The Following Compounds were Prepared by Analogous Method Described Above

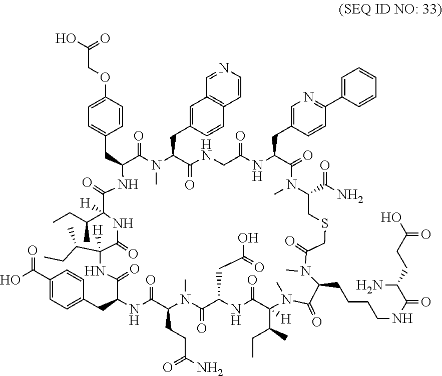 [SMILES: CC[C@H](C)[C@@H]1NC(=O)[C@H](Cc2ccc(C(=O)O)cc2)NC(=O)[C@H](CCC(N)=O)N(C)C(=O)[C@H](CC(=O)O)NC(=O)[C@H]([C@@H](C)CC)N(C)C(=O)[C@H](CCCCNC(=O)[C@H](N)CCC(=O)O)N(C)C(=O)CSC[C@@H](C(N)=O)N(C)C(=O)[C@H](Cc2ccc(-c3ccccc3)nc2)NC(=O)CNC(=O)[C@H](Cc2ccc3ccncc3c2)N(C)C(=O)[C@H](Cc2ccc(OCC(=O)O)cc2)NC(=O)[C@H]([C@@H](C)CC)NC1=O]

The desired macrocyclic peptide was synthesized as outlined in Scheme-P2 on Fmoc-Sieber amide resin (0.50 mmol). Elongation of the linear peptide except for X9 was achieved by Method S-1 employing Fmoc-AAs listed in below table. Coupling reaction of Fmoc-MeNa127N-OH was performed manually as described in Method S4. The obtained peptide on the resin was treated with Boc-de(tBn)-OH (2.1 mmol) in DMF (10 mL)/DIC (4.0 mmol) in DMF (4 mL)/Oxyna pure® (2.0 mmol) in DMF (4 mL) at 75° C. for 30 min as outlined in Step-B2. Subsequently, the resin was shaken in a mixture of Pd(PPh₃)₄ (115.6 mg, 0.2 eq) and PhSiH₃ (616 μL, 10 eq) in DCM (25 mL) at room temperature for 1 hr as outlined in Step-B3. The resin was treated with the reagent mixture (25 mL) and performed the chloroacetylation as described in Step-B4 at room temperature for 30 min. The polypeptide on the resin was treated with the cleavage cocktail (30 mL) for 10 min as outlined in Step-B5 to furnish the linear peptide, which was dissolved in MeCN/H₂O (100 mL). To the mixture was added TEA (697 μL) and then the mixture was shaken for at room temperature for 14 hrs, and then concentrated as outlined in Step-B6. The resulting residue was purified by preparative RP-HPLC. Pure fractions were combined and lyophilized to afford the title macrocyclic peptide. HPLC-MS (Method A-1): 5.74 min, ESI-MS m/z: 982.9 [M+2H]2+.

TABLE 16

| order of Fmoc-AA | AA position in | |
|---|---|---|
| used in the SPPS | the cyclic peptide | Fmoc-AA |
| 1 | X12 | Fmoc-MeC(Trt)-OH |
| 2 | X11 | Fmoc-3Py6Ph-OH |
| 3 | X10 | Fmoc-G-OH |
| 4 | X9 | Fmoc-MeNal27N-OH |
| 5 | X8 | Fmoc-F4aao(tBu)-OH |
| 6 | X7 | Fmoc-I-OH |
| 7 | X6 | Fmoc-I-OH |
| 8 | X5 | Fmoc-F4COO(tBu)-OH |
| 9 | X4 | Fmoc-MeQ(Trt)-OH |
| 10 | X3 | Fmoc-D(Mpe)-OH |
| 11 | X2 | Fmoc-MeI-OH |
| 12 | X1 | Alloc-MeK(Fmoc)-OH |

### Example B10. The Following Peptides were Prepared by Methods Analogously to the Examples Above

TABLE 17

| | SEQ | | Ana- |
|---|---|---|---|
| Ex. | ID | | lytical |
| No. | NO: | Structure | Data |
| 10- 1 | 34 | 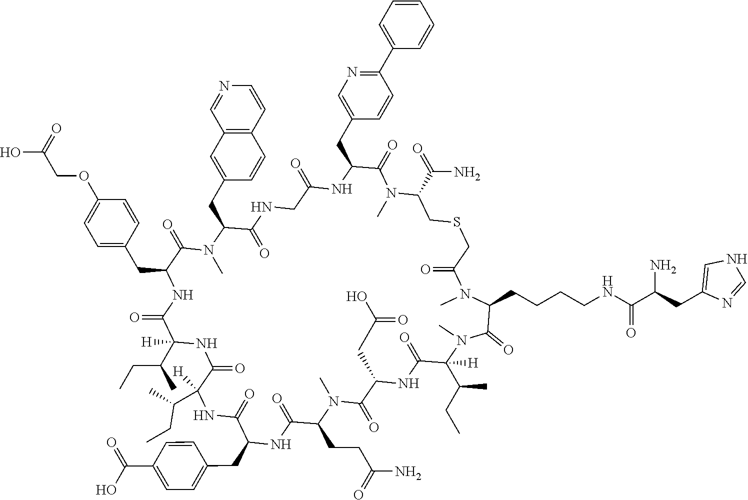 | HPLC- MS (Meth- od A- 1): 5.43 min ESI- MS m/z: 657.7 [M + 3H]3+ |
| 10- 2 | 41 | 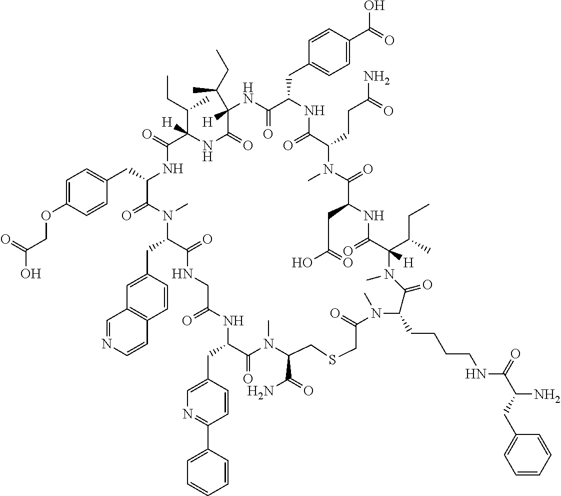 | HPLC- MS (Meth- od A- 2): 5.67 min ESI- MS m/z: 991.4 [M + 2H]2+ |

### Example C: Biological Examples

### Example C1: Biological Example C1

### Macrocyclic Peptides in this Disclosure were Tested for Binding to Glypican-3 Protein by SPR

### Method B-1

A CM3 sensor chip (Cytiva) was inserted into a Biacore T200 (Cytiva), primed three times with a running buffer: HBS-P+, pH 7.4 (Cytiva), and equilibrated at a flow rate of 30 μL/min. Ligand immobilization was performed at a flow rate of 10 μL/min. 50 μL each of 60 mM EDC solution (Cytiva) and 650 mM NHS solution (Cytiva) were mixed, and then reacted at a flow rate of 10 μL/min for 420 seconds, 150 μL of 0.2 μM human Glypican3 (R&D systems, catalog number: 2119-GP) solution which was prepared by diluting with 10 mM acetic acid solution (pH 5.0), was reacted at a flow rate of 10 μL/min for 420 seconds, and Glypican3 was immobilized on the CM3 sensor chip. After immobilization, 1.0 M ethanolamine aqueous solution (Cytiva) was allowed to react for 420 seconds at a flow rate of 10 μL/min for capping. A peptide lysate prepared to 10 mM in DMSO solution was diluted with running buffer to a final concentration of 10 μM peptide lysate. After that, 5 serial dilution series (100 nM, 50 nM, 25 nM, 10 nM, 5 nM) of peptide solutions were prepared. Using the above samples, peptide kinetics for Glypican3 was obtained by SPR measurement. Kinetics evaluation of Glypican3 was performed by SPR measurements at a flow rate of 10 μL/min, setting the association time to 120 seconds and the disassociation time to 600 seconds.

### Method B-2

A CM3 sensor chip (Cytiva) was inserted into a Biacore T200 (Cytiva), primed three times with a running buffer: HBS-P-+, pH 7.4 (Cytiva), and equilibrated at a flow rate of 30 μL/min. Using the Anti-Fc Capture Kit (Cytiva), 50 LL each of 60 mM EDC solution (Cytiva) and 650 mM NHS solution (Cytiva) were mixed, and then reacted at a flow rate of 10 μL/min for 420 seconds, 150 μL of 20 μL/mL Anti-human IgG (Fc) Antibody solution was prepared by diluting with 10 mM acetic acid solution (pH 5.0), reacted at a flow rate of 10 μL/min for 420 seconds, and immobilized on a CM3 sensor chip. After immobilization, 1.0 M ethanolamine aqueous solution (Cytiva) was allowed to react for 420 seconds at a flow rate of 10 μL/min for capping. Human Glypican3-Fc (ACRO Biosystems, GP3-H5258) was allowed to react for 60 seconds at a flow rate of 10 μL/min. After capturing human Glypican3-Fc, a peptide solution prepared to 10 mM in DMSO solution was diluted with a running buffer to a final concentration of 10 μM. After that, 5 serial dilution series (100 nM, 50 nM, 25 nM, 10 nM, 5 nM) of peptide solutions were prepared. Using the above samples, peptide kinetics for Glypican3 was obtained by SPR measurement. Kinetics evaluation of Glypican3 was performed by SPR measurements at a flow rate of 10 μL/min. setting the association time to 120 seconds and the disassociation time to 600 seconds.

### Method B-3

A Protein A sensor chip (Cytiva) was inserted into a Biacore T200 (Cytiva), priming was performed three times with a running buffer: HBS-P+, pH 7.4 (Cytiva), and equilibration was carried out at a flow rate of 30 μL/min. Glypican3-Fc was allowed to react for 60 seconds at a flow rate of 10 μL/min. After capturing human Glypican3-Fe, a peptide solution prepared to 10 mM in DMSO solution was diluted with a running buffer to a final concentration of 10 μM. After that, 5 serial dilution series (100 nM, 50 nM, 25 nM, 10 nM, 5 nM) of peptide solutions were prepared. Using the above samples, peptide kinetics for Glypican3 was obtained by SPR measurement. Kinetics evaluation of Glypican3 was performed by SPR measurements at a flow rate of 10 LL/min, setting the association time to 120 seconds and the disassociation time to 600 seconds.

### Method B-4

A Protein A sensor chip (Cytiva) was inserted into a Biacore T200 (Cytiva), priming was performed three times with a running buffer: HBS-P+, pH 7.4 (Cytiva), and equilibration was carried out at a flow rate of 30 L/min. Gly pican3-Fc was allowed to react for 60 seconds at a flow rate of 10 μL/min. After capturing human Glypican3-Fc, a peptide solution prepared to 1 mM in DMSO solution was diluted with a running buffer to a final concentration of 1 μM. After that, the peptide solution was diluted to 40 nM in a similar manner. Using the above samples, peptide kinetics for Glypican3 was obtained by SPR measurement. Kinetics evaluation of Glypican3 was performed by SPR measurements at a flow rate of 30 μL/min. setting the association time to 120 seconds and the disassociation time to 600 seconds.

The obtained data by Method B-1, Method B-2, Method B-3 and Method B-4 were analyzed by follow. Single Cycle Kinetics was used as the kinetics evaluation model, and curve fitting was performed using Biacore T200 Evaluation Software Version 3.0 (Cytiva). The obtained sensorgram was subjected to curve fitting by the method of least squares and obtained KD values of the macrocyclic peptides in this disclosure for Glypican 3 protein are shown in Table 18.

TABLE 18

| Example | SEQ | Conjugate | SPR KD | SPR |
|---|---|---|---|---|
| No. | ID No. | ID | (nM) | method |
| — | 72 | Lu-C-72 | 0.36 | B-3 |
| — | 45 | Lu-C-45 | 0.74 | B-2 |
| — | 1 | Lu-C-1 | 0.29 | B-1 |
| — | 21 | Lu-C-21 | 1.04 | B-2 |
| — | 22 | Lu-C-22 | 1.99 | B-2 |
| — | 24 | Lu-C-24 | 4.03 | B-2 |
| — | 28 | Lu-C-28 | 1.80 | B-2 |
| — | 26 | Lu-C-26 | 2.52 | B-2 |
| — | 30 | Lu-C-30 | 0.70 | B-2 |
| — | 31 | Lu-C-31 | 7.05 | B-2 |
| — | 32 | Lu-C-32 | 16.50 | B-2 |
| — | 33 | Lu-C-33 | 0.87 | B-2 |
| — | 34 | Lu-C-34 | 0.80 | B-2 |
| — | 35 | Lu-C-35 | 2.03 | B-2 |
| — | 44 | Lu-C-44 | 1.00 | B-2 |
| — | 41 | Lu-C-41 | 0.80 | B-2 |
| — | 42 | Lu-C-42 | 0.67 | B-2 |
| — | 64 | Lu-C-64 | 0.53 | B-3 |
| — | 62 | Lu-C-62 | 0.15 | B-3 |
| — | 63 | Lu-C-63 | 1.6 | B-3 |
| 1 | 45 | — | 0.2 | B-3 |
| 2-1 | 2 | — | 0.5 | B-1 |
| 2-2 | 3 | — | 1.5 | B-1 |
| 2-3 | 4 | — | 1.2 | B-1 |
| 2-4 | 7 | — | 0.3 | B-3 |
| 2-5 | 8 | — | 2.6 | B-1 |
| 2-6 | 9 | — | 2.7 | B-1 |
| 2-7 | 10 | — | 6.8 | B-1 |
| 2-8 | 11 | — | 1.3 | B-1 |
| 2-9 | 12 | — | 7.2 | B-1 |
| 2-10 | 13 | — | 2.2 | B-1 |
| 2-11 | 14 | — | 2.5 | B-1 |
| 2-12 | 15 | — | 2.9 | B-1 |
| 2-13 | 16 | — | 2.8 | B-1 |
| 2-14 | 17 | — | 4.8 | B-1 |
| 2-15 | 18 | — | 1.8 | B-1 |
| 2-16 | 19 | — | 3.5 | B-1 |
| 2-17 | 20 | — | 1.9 | B-1 |
| 2-18 | 36 | — | 3.8 | B-2 |
| 2-19 | 37 | — | 0.5 | B-2 |
| 2-20 | 38 | — | 0.7 | B-2 |
| 2-21 | 39 | — | 0.4 | B-2 |
| 2-22 | 40 | — | 0.8 | B-2 |
| 2-23 | 43 | — | 1.5 | B-2 |
| 2-24 | 58 | — | 0.5 | B-3 |
| 2-25 | 59 | — | 6.6 | B-3 |
| 2-26 | 30 | — | 0.3 | B-3 |
| 2-27 | 35 | — | 1.1 | B-3 |
| 2-28 | 44 | — | 0.3 | B-3 |
| 2-29 | 46 | — | 1.0 | B-3 |
| 2-30 | 47 | — | 6.6 | B-3 |
| 2-31 | 48 | — | 1.2 | B-3 |
| 2-32 | 49 | — | 1.8 | B-3 |
| 2-33 | 50 | — | 0.3 | B-3 |
| 2-34 | 51 | — | — | — |
| 2-35 | 52 | — | 0.6 | B-3 |
| 2-36 | 53 | — | — | — |
| 2-37 | 54 | — | 1.0 | B-3 |
| 2-38 | 55 | — | 1.8 | B-3 |
| 2-39 | 67 | — | 25.3 | B-3 |
| 2-40 | 66 | — | 21.5 | B-3 |
| 2-41 | 68 | — | 58.5 | B-3 |
| 2-42 | 31 | — | 3.2 | B-3 |
| 2-43 | 32 | — | 9.3 | B-3 |
| 2-44 | 69 | — | 49.1 | B-3 |
| 2-45 | 5 | — | — | — |
| 2-46 | 6 | — | 0.2 | B-3 |
| 2-47 | 21 | — | 0.2 | B-3 |
| 2-48 | 22 | — | 0.5 | B-3 |
| 2-49 | 23 | — | — | — |
| 2-50 | 24 | — | 2.0 | B-3 |
| 2-51 | 25 | — | 2.5 | B-3 |
| 2-52 | 26 | — | 1.6 | B-3 |
| 2-53 | 28 | — | 1.0 | B-3 |
| 2-54 | 29 | — | 0.1 | B-3 |
| 2-55 | 56 | — | 5.3 | B-3 |
| 2-56 | 27 | — | 7.9 | B-3 |
| 2-57 | 1 | — | 0.17 | B-3 |
| 2-58 | 61 | — | 1.19 | B-3 |
| 2-59 | 63 | — | 1.55 | B-3 |
| 2-60 | 62 | — | 0.23 | B-3 |
| 2-61 | 72 | — | 0.23 | B-4 |
| 3 | 1 | PLB-1 | 0.25 | B-3 |
| 4-1 | 35 | PLB-35 | 3.63 | B-3 |
| 4-2 | 28 | PLB-28 | 1.53 | B-3 |
| 4-3 | 64 | PLB-64 | 1.04 | B-3 |
| 4-4 | 30 | PLB-30 | 0.75 | B-3 |
| 5 | 60 | PLB-60 | 0.11 | B-3 |
| 6 | 45 | PLD-45 | 0.88 | B-3 |
| 7-1 | 30 | PLD-30 | 1.08 | B-3 |
| 7-2 | 1 | PLD-1 | 0.32 | B-3 |
| 8 | 42 | — | 0.57 | B-3 |
| 9 | 33 | — | 0.52 | B-3 |
| 10-1 | 34 | — | 0.51 | B-3 |
| 10-2 | 41 | — | 0.54 | B-3 |
| 7-3 | | Mod3 | 0.90 | B-3 |
| 7-4 | | Mod5 | 3.6 | B-3 |
| 7-5 | | Mod6 | 0.24 | B-3 |
| 7-6 | | Mod7 | 4.2 | B-3 |

### Example C2: Biological Example C2

Macrocyclic peptide-conjugates in this disclosure were tested in cells expressing glypican 3 and analyzed by flow cytometry.

### Cell Lines

HepG2 (human hepatocellular carcinoma) and SK-HEP-1 (human hepatic adenocarcinoma) cell lines were purchased from the American Type Culture Collection (ATCC; Manassas, VA). Huh-7 (human hepatoblastoma) cell line was purchased from the Japanese Collection of Research Bioresources Cell Bank (JCRB Cell Bank, Osaka, Japan). HepG2 and SK-HEP-1 were grown in EMEM (FUJIFILM Wako Pure Chemical Corporation, 055-08975) with 10% fetal bovine serum (FBS; Thermo Fisher Scientific, 10270106) and 0.1% gentamicin (NACALAI TESQUE, 11980-14). Huh-7 was grown in DMEM (Thermo Fisher Scientific, 10567014) with 10% FBS and 0.1% gentamicin.

### Flow Cytometry

HepG2, SK-HEP-1 and Huh-7 cells were collected from the culture dish and stained with biotinylated peptides at 100 nM followed by Streptavidin, R-Phycoerythrin Conjugate (SAPE: Thermo Fisher Scientific, S866) (1:250). Cells were washed with phosphate-buffered saline (PBS; FUJIFILM Wako Pure Chemical Corporation, 043-29791) containing 0.5% FBS after addition of peptides or SAPE. All staining procedures were performed on ice. Samples were measured using CytoFLEX S (Beckman Coulter) and CytExpert ver2.4 software. All data were analyzed using FlowJo™ (BD Bioscience). For discrimination of live cells from dead cells, SYTOX™ Blue Dead Cell Stain (Thermo Fisher Scientific, S34857) (1:5000) was added prior to measurement.

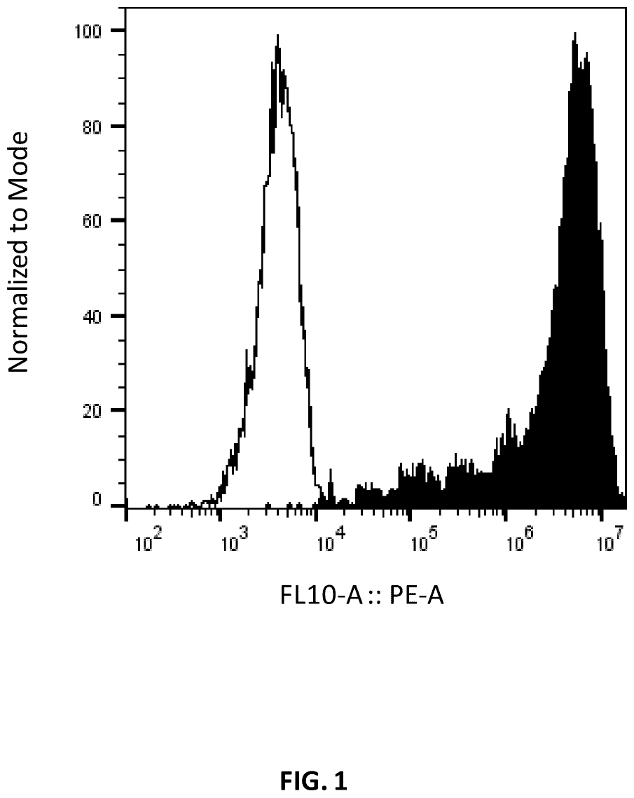

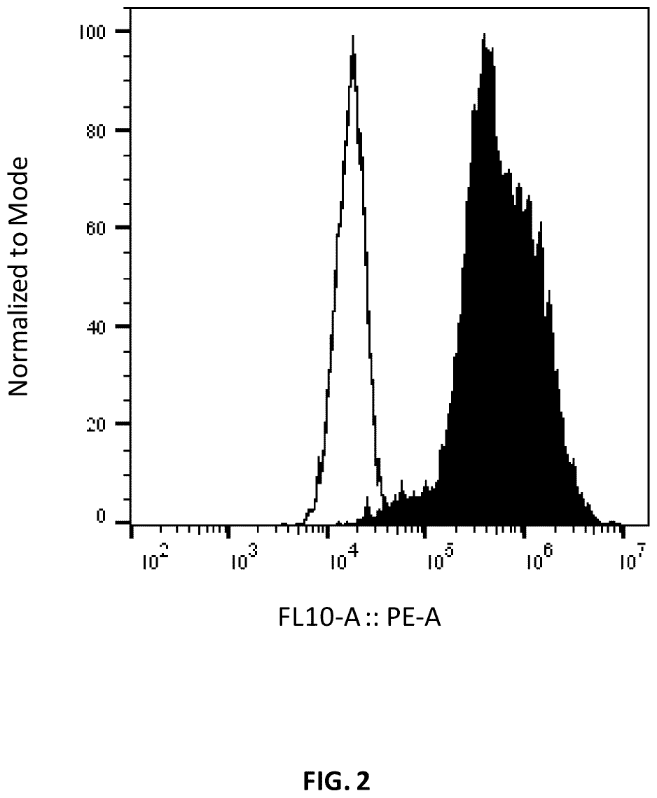

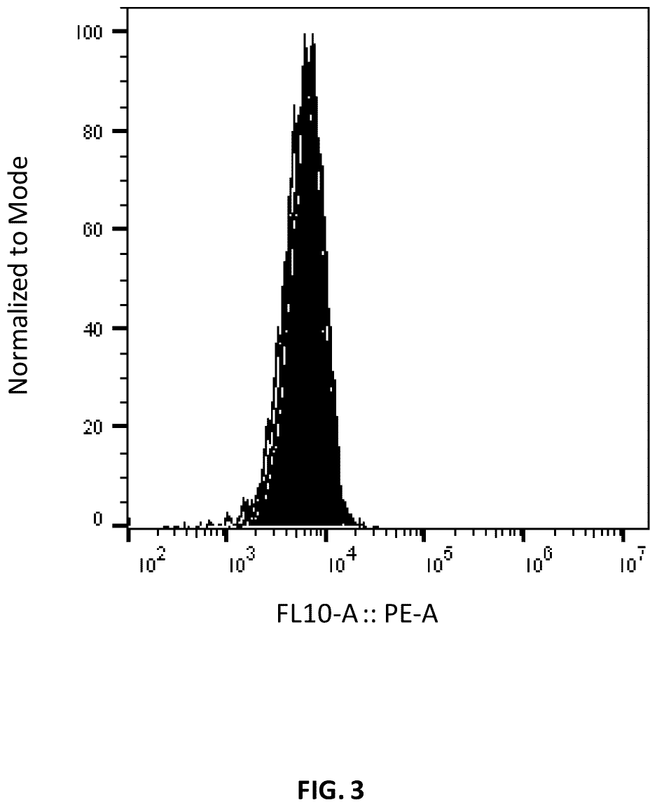

The glypican-3 binding macrocyclic peptide-biotin conjugate in Example B5 (Conjugate ID No.: PLB-60) was tested in the condition above. FIGS. 1-3 showed the flow charts of the comparative staining of HepG2 (glypican 3 expressed on the cell surface), Huh-7 (glypican 3 expressed on the cell surface), and SK-Hep1 (negative control) respectively, indicating specific binding to both glypican expressing cells and SAPE.

SulfoCy5-peptides Flow cytometry

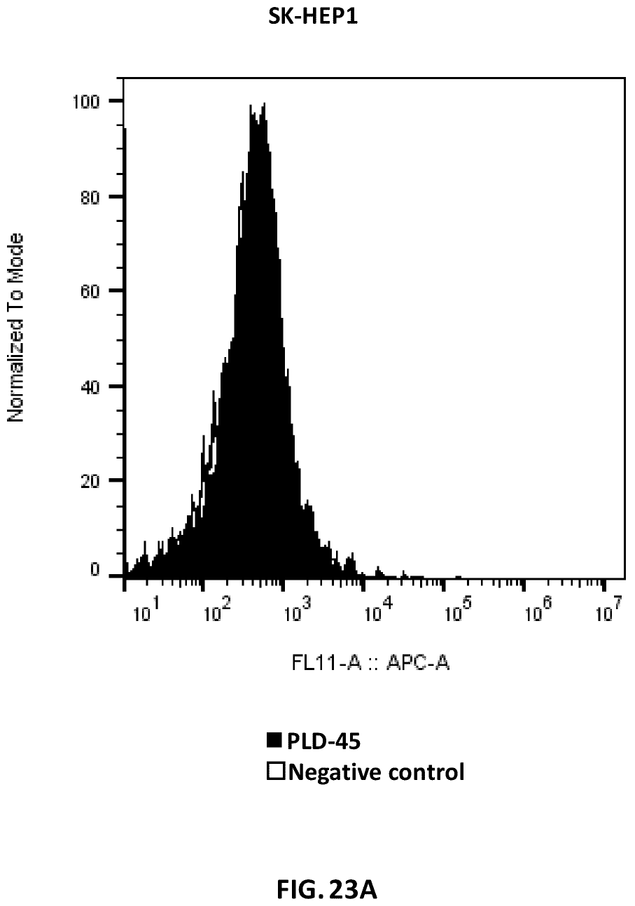

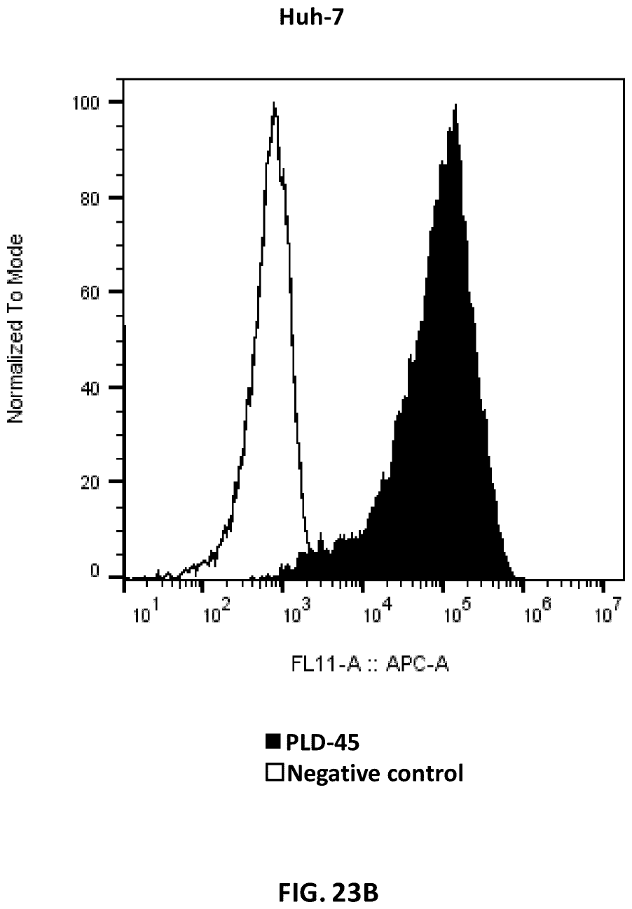

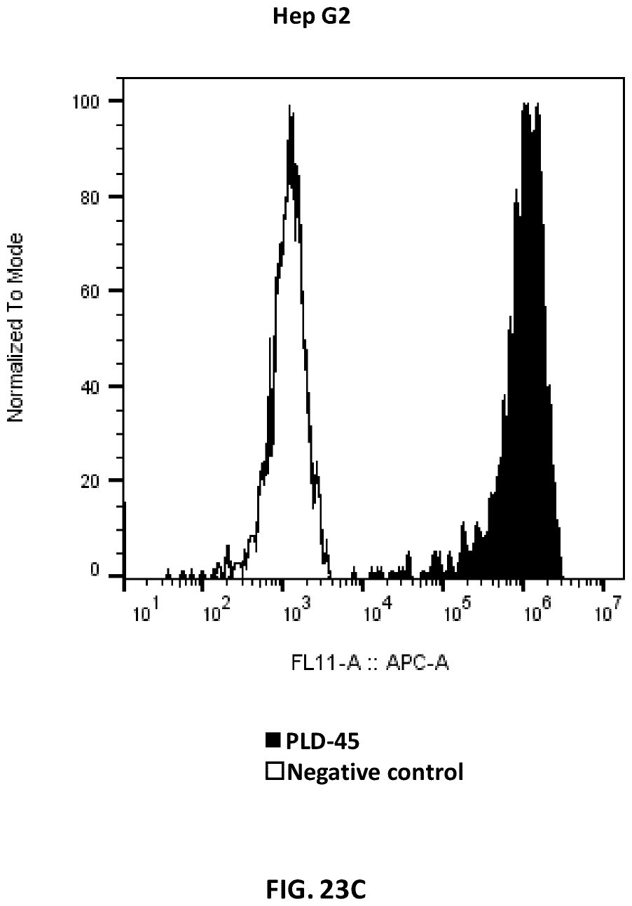

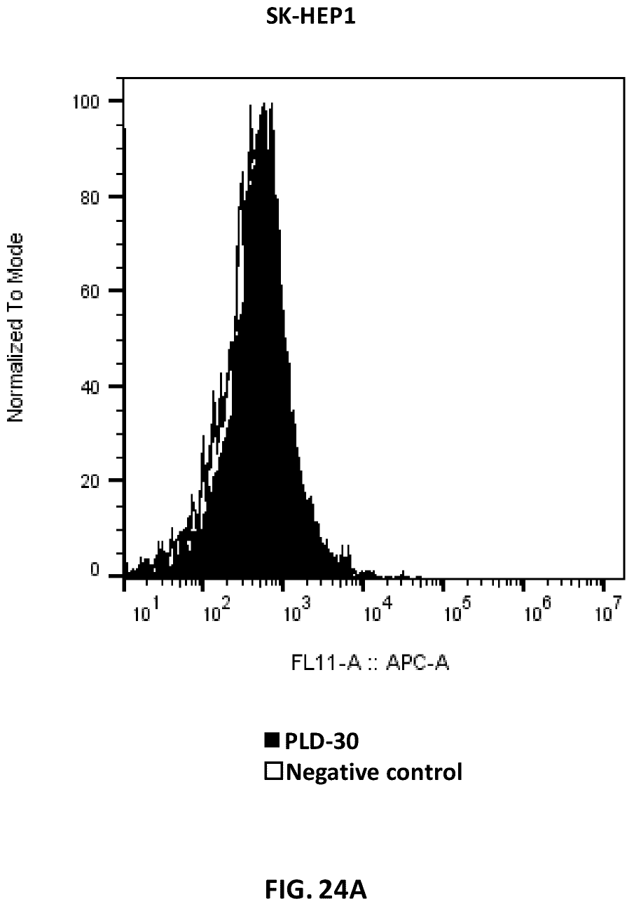

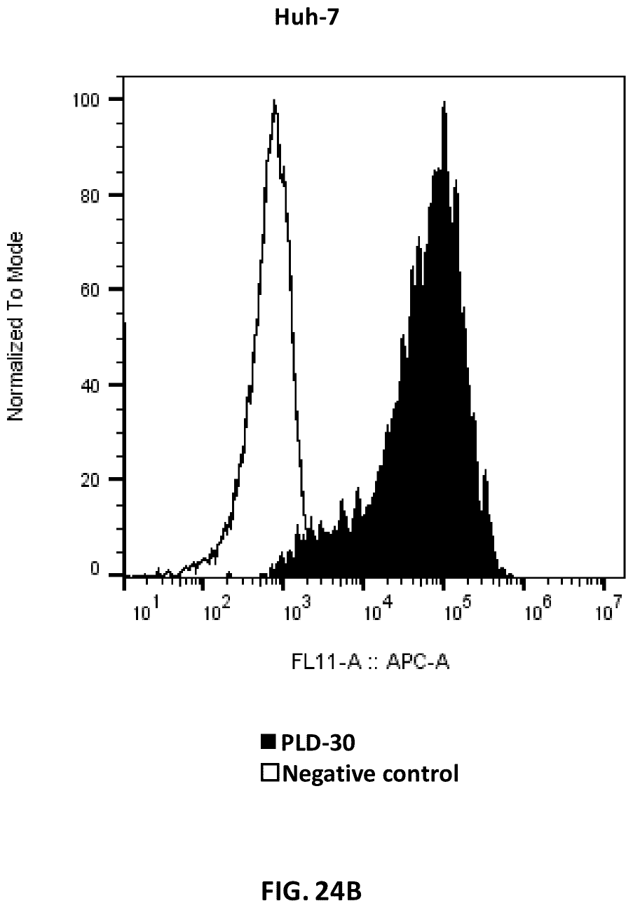

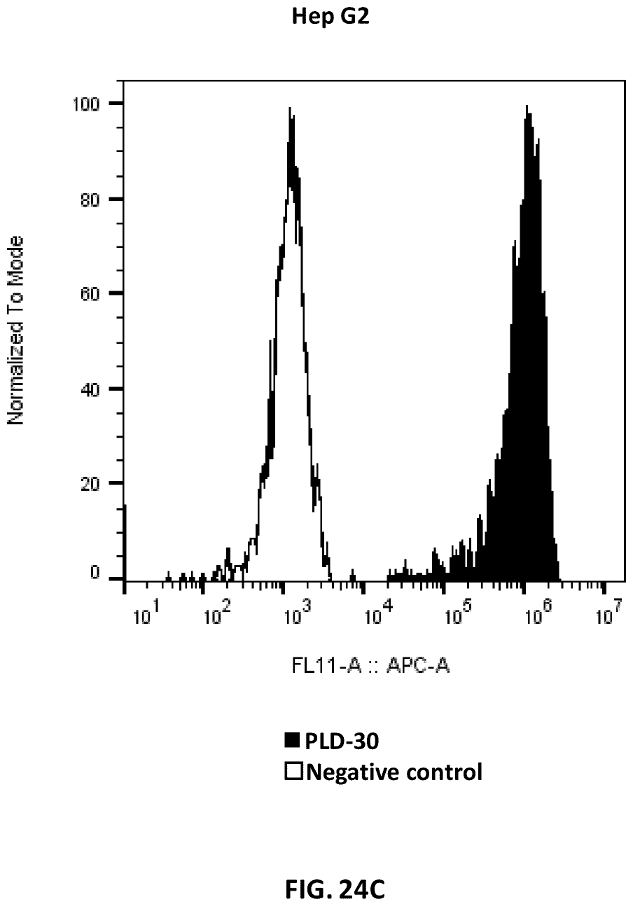

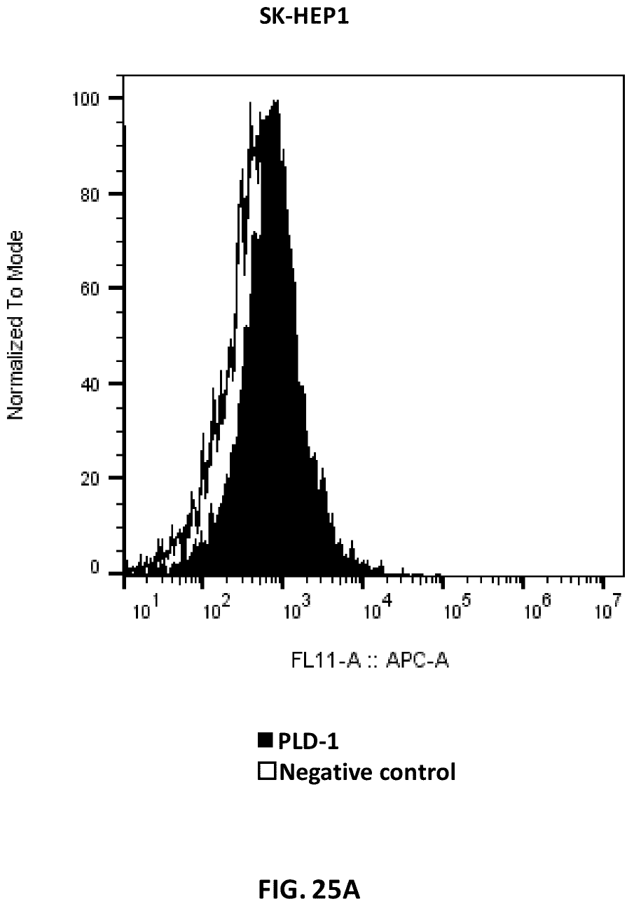

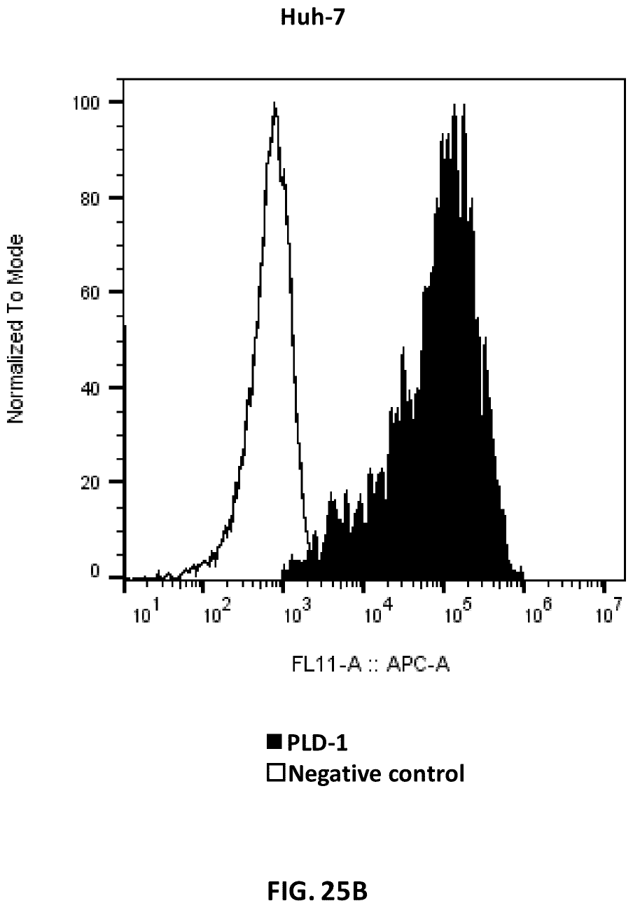

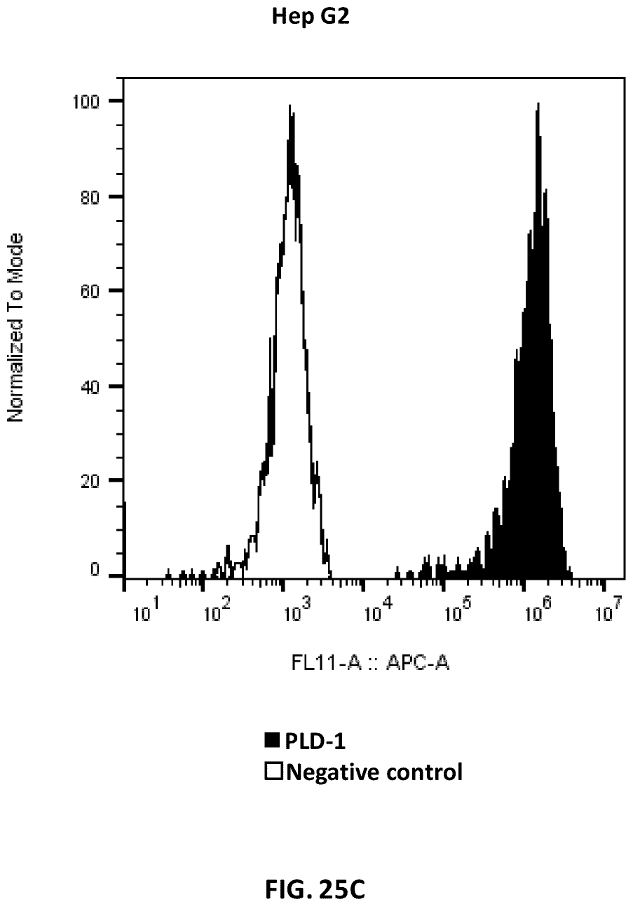

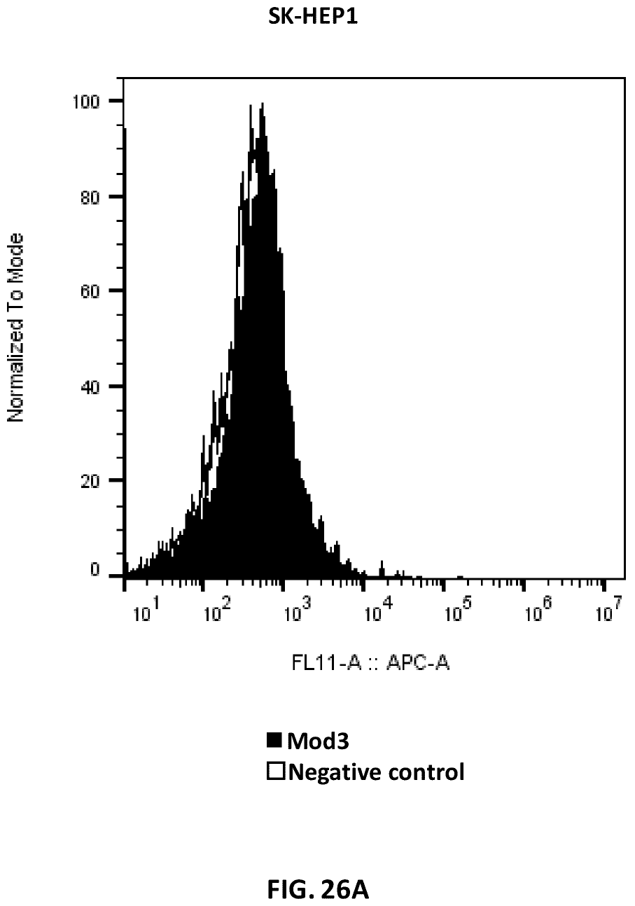

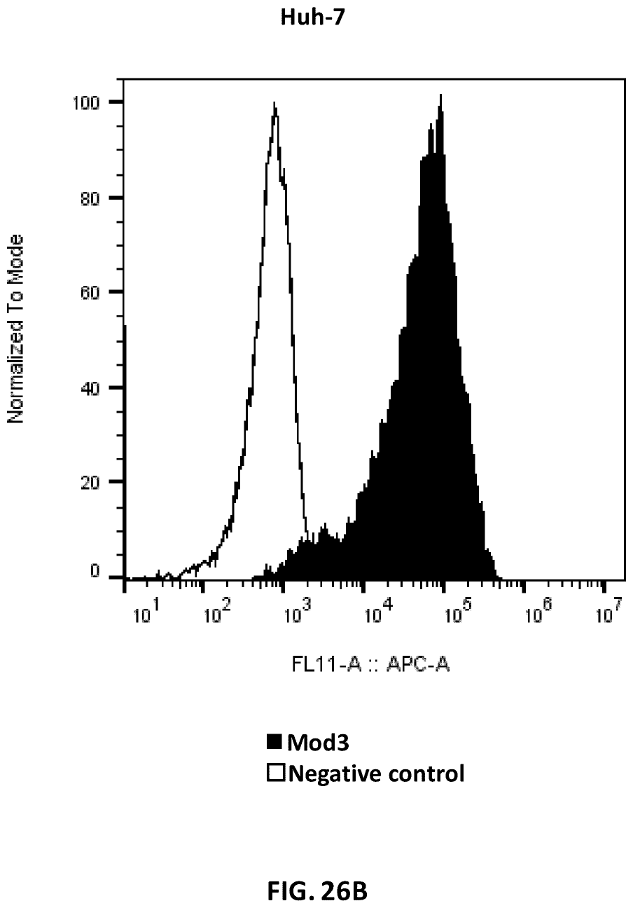

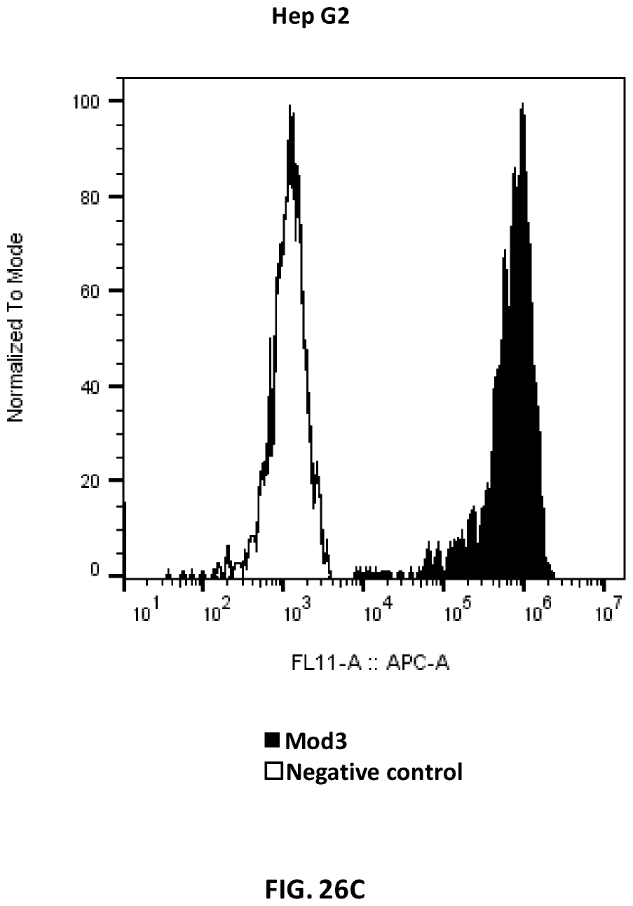

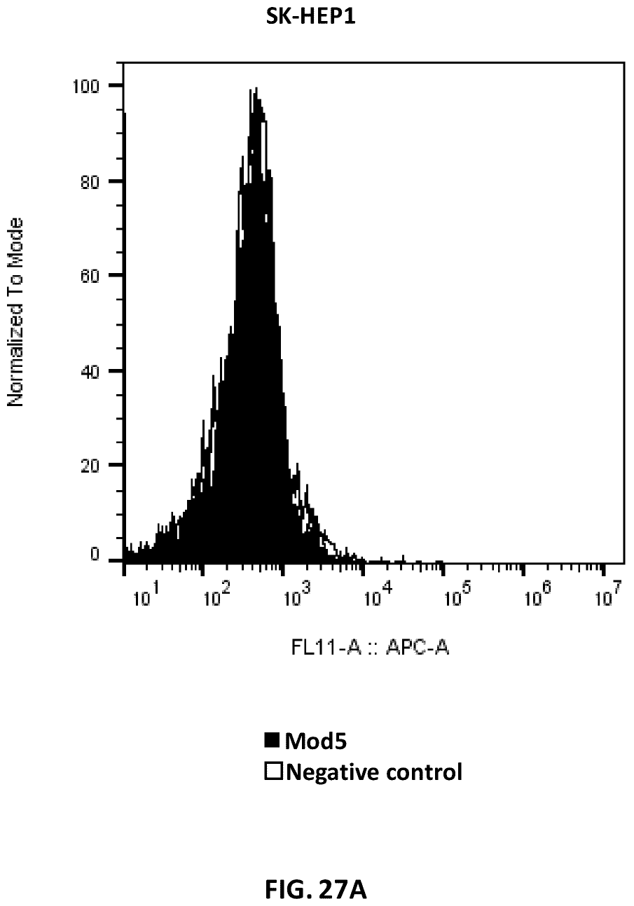

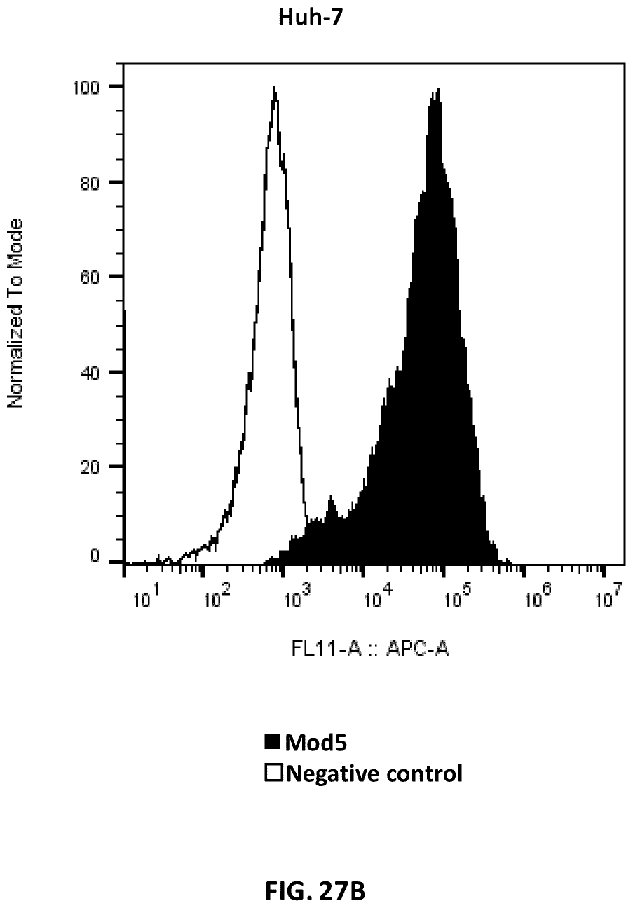

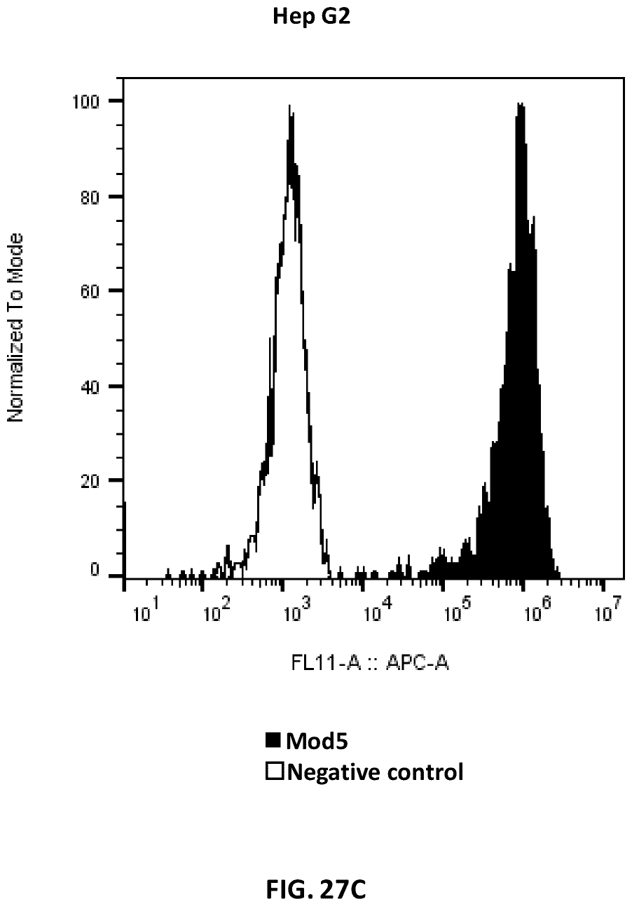

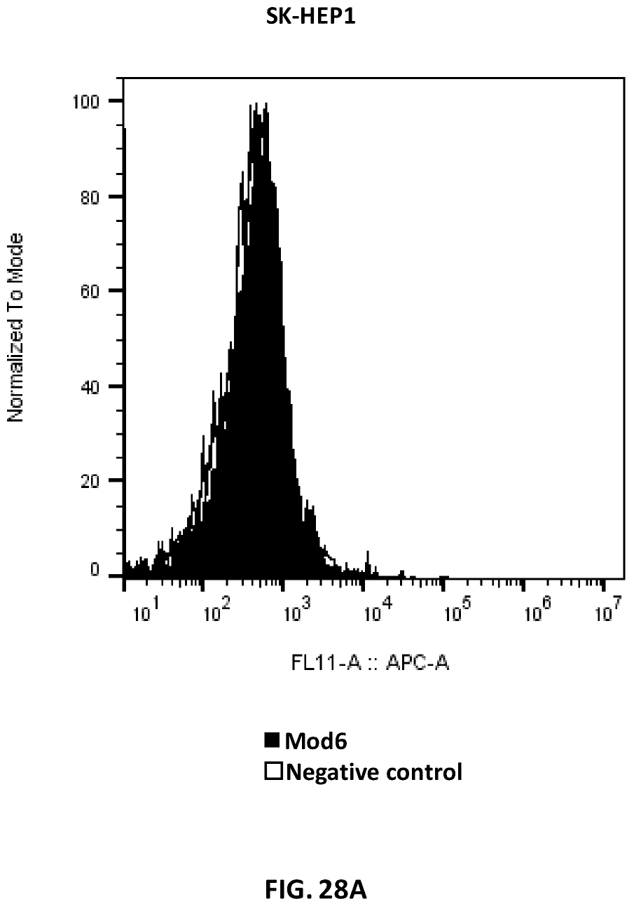

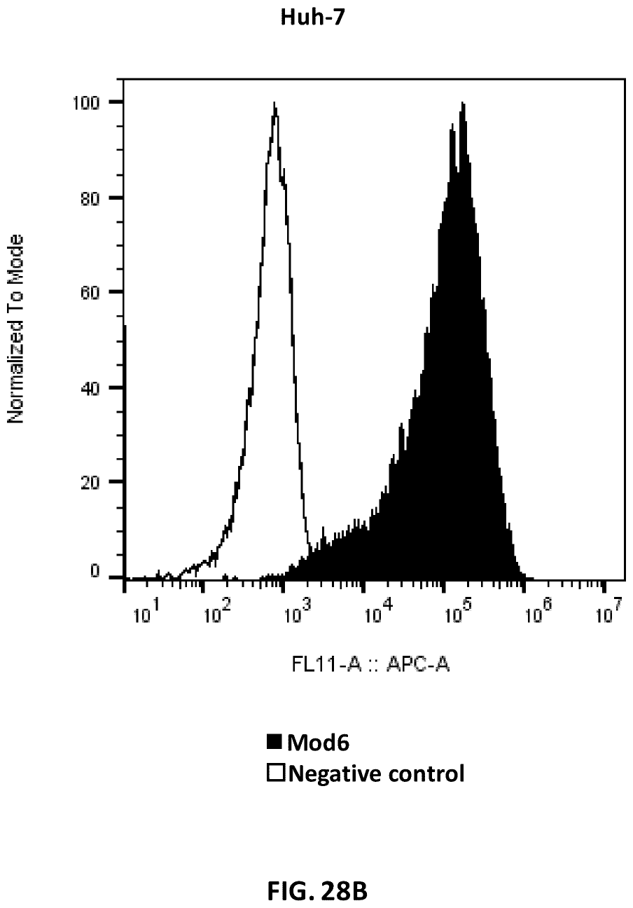

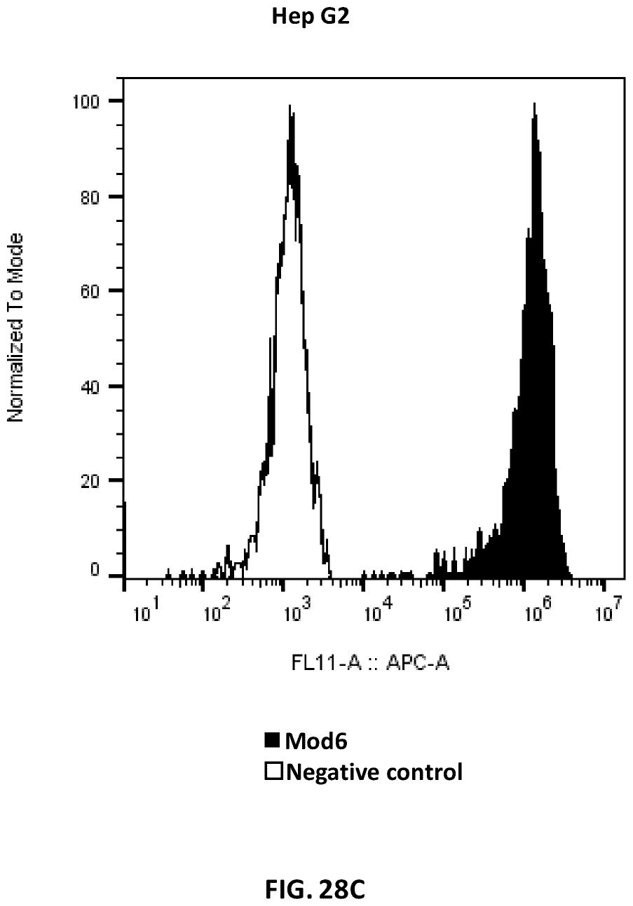

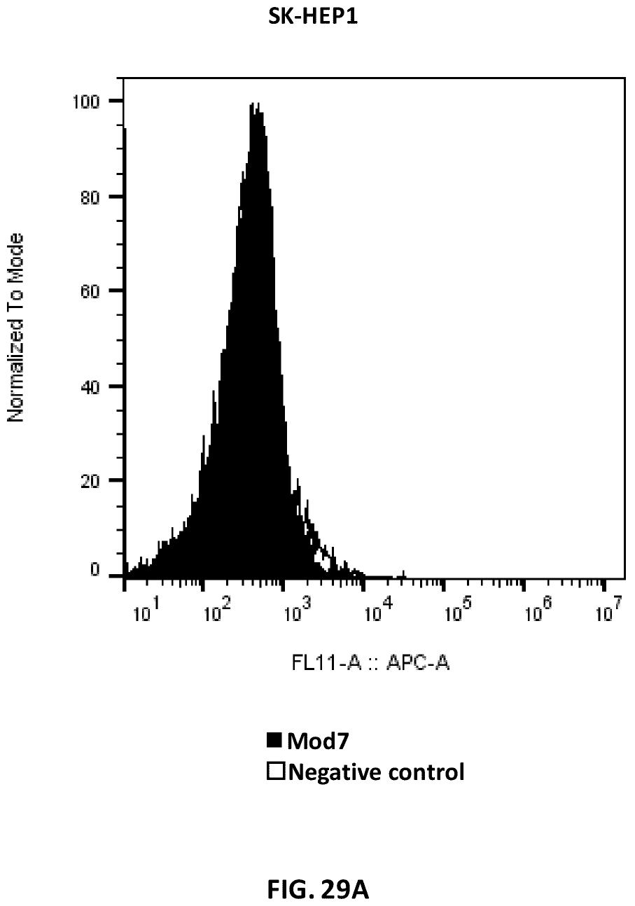

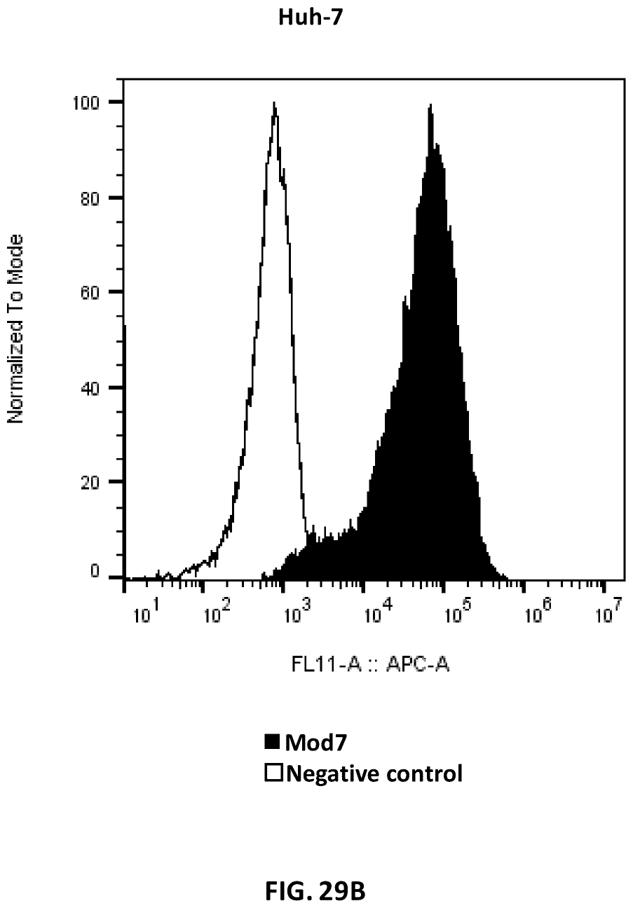

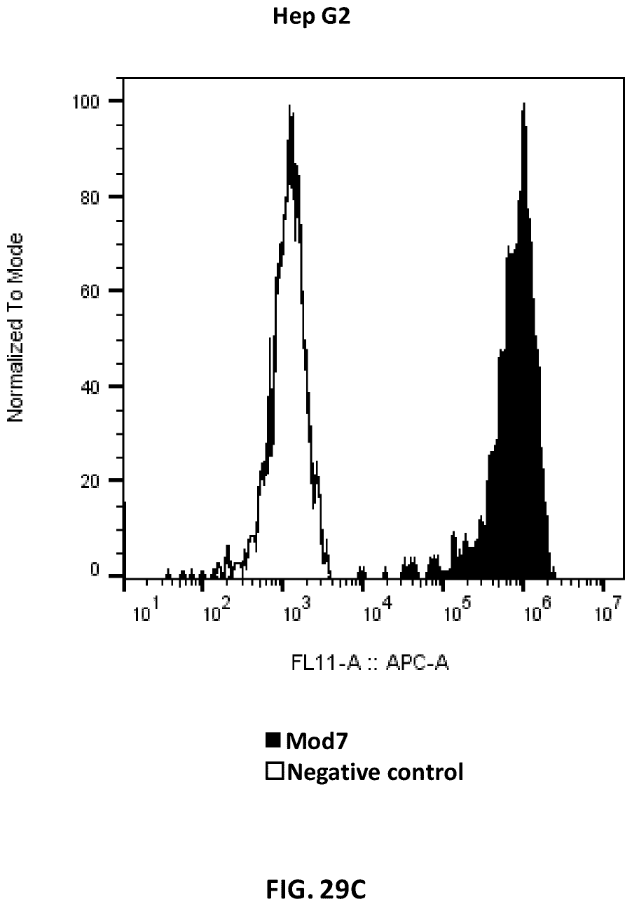

HepG2, SK-HEP-1 and Huh-7 cells were collected from the culture dish and stained with SulfoCy5-peptides at 100 nM. Cells were washed with phosphate-buffered saline (PBS; FUJIFILM Wako Pure Chemical Corporation, 043-29791) containing 0.5% FBS after addition of SulfoCy5-peptides. All staining procedures were performed on ice. Samples were measured using CytoFLEX S (Beckman Coulter) and CytExpert ver2.4 software. All data were analyzed using FlowJo™ (BD Bioscience). For discrimination of live cells from dead cells, SY TOX™ Blue Dead Cell Stain (Thermo Fisher Scientific, S34857) (1:5000) was added prior to measurement. The glypican-3 binding macrocyclic SulfoCy5-peptides conjugate in Example B6 and Example B7 were tested in the condition above. FIGS. 23A-29C show the flow charts of the comparative staining of HepG2 (glypican 3 expressed on the cell surface), Huh-7 (glypican 3 expressed on the cell surface), and SK-Hep1 (negative control), indicating specific binding to both glypican expressing cells.

Certain linker-added peptides have identical or substantially the same peptide sequences compared to certain conjugates comprising such linker-added peptides, or “naked” peptides without the linker. Although the GPC3 binding affinity for such linker-added peptides have not been directly tested, they likely bind to GPC3 with similar avidity compared to that of the conjugates comprising such linker-added peptides, or that of the “naked” peptides without the linker portion.

The present technology may be used in bio-related industries and the pharmaceutical industry.

All references cited in this specification, and their references, are incorporated by reference herein in their entirety where appropriate for teachings of additional or alternative details, features, and/or technical background.

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations, or improvements therein may be subsequently made by those skilled in the art which are also intended to be encompassed by the following claims.

1-76. (canceled)

77. A radiopharmaceutical of the following structure: 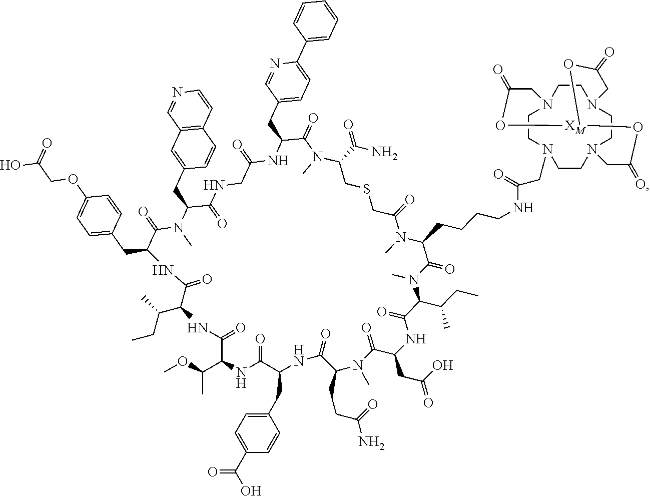
or a pharmaceutically acceptable salt thereof, wherein X_{M} is an alpha particle-emitting radionuclide or a beta particle-emitting radionuclide.

78. The radiopharmaceutical of claim 77, wherein X_{M} is an alpha particle-emitting radionuclide.

79. The radiopharmaceutical of claim 78, wherein the alpha particle-emitting radionuclide is Ac-225, Bi-213, Bi-209, Tb-149, Ra-223, Th-227, Fr-223, Gd-148, Th-229, Pb-212, or Po-213.

80. The radiopharmaceutical of claim 77, wherein X_{M} is Ac-225.

81. The radiopharmaceutical of claim 77, of the following structure: 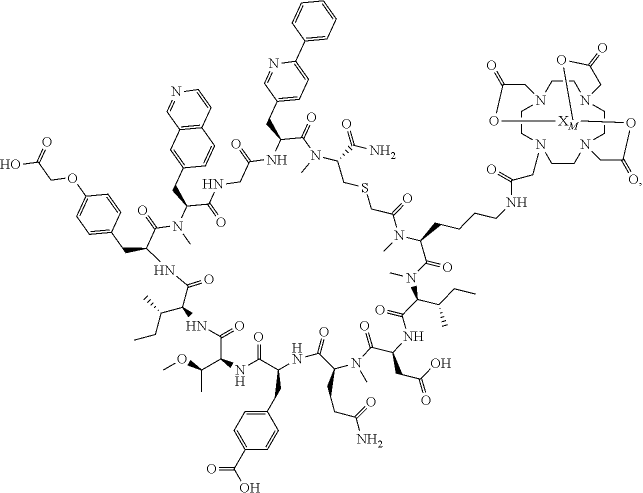
wherein X_{M} is Ac-225.

82. The radiopharmaceutical of claim 77, wherein the radionuclide is a beta particle-emitting radionuclide.

83. The radiopharmaceutical of claim 82, wherein the beta particle-emitting radionuclide is Cu-67, Lu-177, Y-90, Rh-105, Yb-175, Tm-167, Pm-153, or Sm-153.

84. The radiopharmaceutical of claim 83, wherein the beta particle-emitting radionuclide is Lu-177.

85. A pharmaceutical composition comprising the radiopharmaceutical or pharmaceutically acceptable salt thereof of claim 77 and a pharmaceutically acceptable excipient or carrier.

86. A pharmaceutical composition comprising the radiopharmaceutical or pharmaceutically acceptable salt thereof of claim 80 and a pharmaceutically acceptable excipient or carrier.

87. A pharmaceutical composition comprising the radiopharmaceutical of claim 81 and a pharmaceutically acceptable excipient or carrier.

88. A method of treating hepatocellular carcinoma or hepatoblastoma in a subject in need of treatment, the method comprising administering to the subject a therapeutically effective amount of a radiopharmaceutical or pharmaceutically acceptable salt thereof of claim 77.

89. A radiopharmaceutical of the following structure: 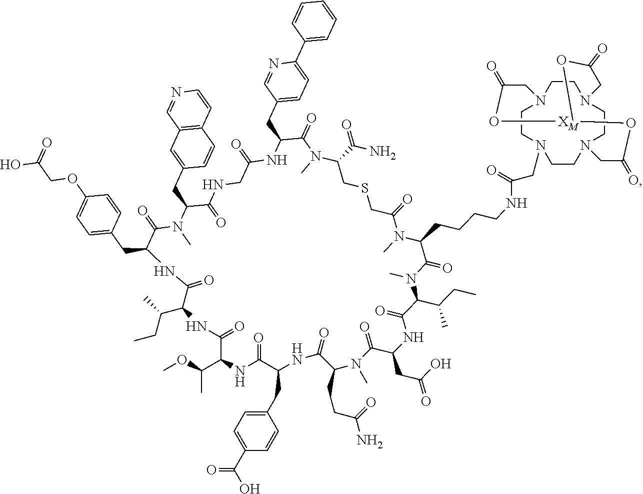
or a pharmaceutically acceptable salt thereof, wherein X_{M} is a positron-emitting radionuclide.

90. The radiopharmaceutical of claim 89, wherein the positron-emitting radionuclide is Ga-68, Cu-62, Cu-64, Zr-89, or Tb-152.

91. The radiopharmaceutical of claim 89, wherein X_{M} is Ga-68.

92. The radiopharmaceutical of claim 89, of the following structure: 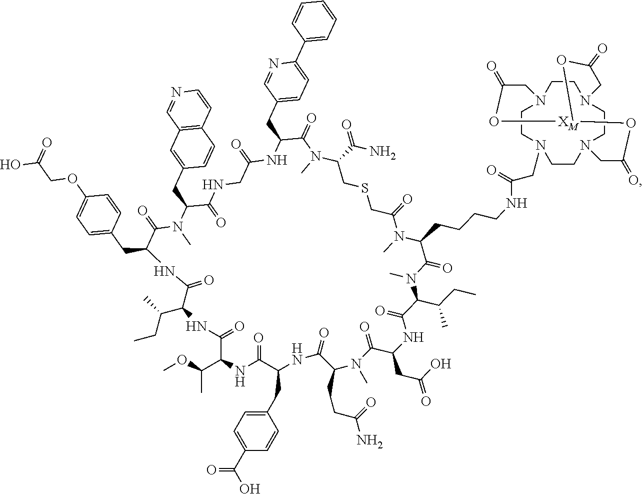
wherein X_{M} is Ga-68.

93. A pharmaceutical composition comprising the radiopharmaceutical or pharmaceutically acceptable salt thereof of claim 89 and a pharmaceutically acceptable excipient or carrier.

94. A pharmaceutical composition comprising the radiopharmaceutical or pharmaceutically acceptable salt thereof of claim 91 and a pharmaceutically acceptable excipient or carrier.

95. A pharmaceutical composition comprising the radiopharmaceutical of claim 92 and a pharmaceutically acceptable excipient or carrier.

96. A method of imaging hepatocellular carcinoma (HCC) or hepatoblastoma in a subject in need thereof, comprising administering to the subject a radiopharmaceutical or pharmaceutically acceptable salt thereof of claim 89; and imaging the HCC or hepatoblastoma in the subject.

97. A conjugate having the structure: 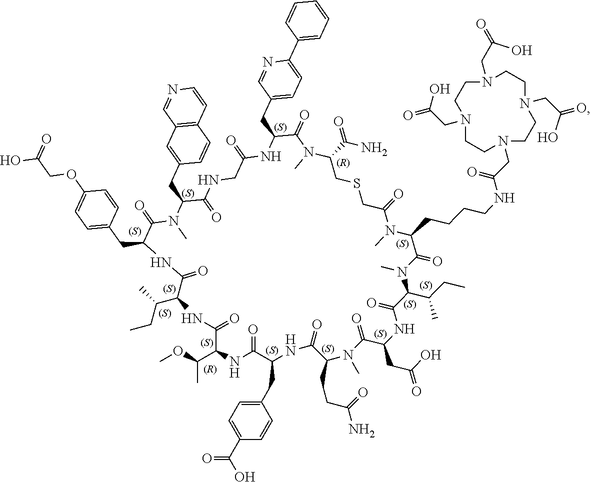
or a pharmaceutically acceptable salt thereof.


SEQUENCE LISTING (406 sequences)

SEQ ID NO: 1 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = methyl-L-Cysteine
  SITE 1..12: note = Optionally cyclic
KIDQFIIYFGXC

SEQ ID NO: 2 - protein, 12 aa, synthetic construct
  SITE 1: note = acetylated methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 3 - protein, 12 aa, synthetic construct
  SITE 1: note = acetylated methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KIDQFIIFFGFC

SEQ ID NO: 4 - protein, 12 aa, synthetic construct
  SITE 1: note = acetylated methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KIQQFIIFFGFC

SEQ ID NO: 5 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KIDQFIIFFGFC

SEQ ID NO: 6 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KIQQFIIFFGFC

SEQ ID NO: 7 - protein, 12 aa, synthetic construct
  SITE 1: note = acetylated methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIYFGXC

SEQ ID NO: 8 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFIIFFGFC

SEQ ID NO: 9 - protein, 12 aa, synthetic construct
  SITE 1: note = methyl-L-Alanine
  SITE 2: note = methyl-L-Glutamine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
AQDQFIIFFGFC

SEQ ID NO: 10 - protein, 12 aa, synthetic construct
  SITE 1: note = methyl-L-Glutamine
  SITE 2: note = methyl-L-Leucine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QLDQFIIFFGFC

SEQ ID NO: 11 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QSDQFIIFFGFC

SEQ ID NO: 12 - protein, 12 aa, synthetic construct
  SITE 1: note = methyl-L-Glutamine
  SITE 2: note = 2-amino-4-methoxybutanoic acid
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QSDQFIIFFGFC

SEQ ID NO: 13 - protein, 12 aa, synthetic construct
  SITE 1: note = methyl-L-Glutamine
  SITE 2: note = methyl-L-Glutamine
  SITE 3: note = 2,6-diamino-6-oxohexanoic acid
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQQQFIIFFGFC

SEQ ID NO: 14 - protein, 12 aa, synthetic construct
  SITE 1: note = methyl-L-Glutamine
  SITE 2: note = methyl-L-Glutamine
  SITE 3: note = 2-amino-6-acetamidohexanoic acid
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQKQFIIFFGFC

SEQ ID NO: 15 - protein, 12 aa, synthetic construct
  SITE 1: note = methyl-L-Glutamine
  SITE 2: note = methyl-L-Glutamine
  SITE 4: note = 3-(1-(carboxymethyl)piperidin-4-yl)-2-(methylamino) propanoic acid
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDAFIIFFGFC

SEQ ID NO: 16 - protein, 12 aa, synthetic construct
  SITE 1: note = methyl-L-Glutamine
  SITE 2: note = methyl-L-Glutamine
  SITE 4: note = Methyl-L-Aspartic Acid
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDDFIIFFGFC

SEQ ID NO: 17 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 2-amino-3-(4-(carboxymethoxy)phenyl)propanoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFIIFFGFC

SEQ ID NO: 18 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 4-(2-amino-2-carboxyethyl)benzoic acid
  SITE 6: note = O-methyl-Threonine
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFTIFFGFC

SEQ ID NO: 19 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFIIFFGFC

SEQ ID NO: 20 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFIIFFGFC

SEQ ID NO: 21 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFIIFFGFC

SEQ ID NO: 22 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KSDQFIIFFGFC

SEQ ID NO: 23 - protein, 12 aa, synthetic construct
  SITE 1: note = methyl-L-lysine
  SITE 2: note = 4-methoxy-2-(methylamino)butanoic acid
  SITE 4: note = 3-(1-(carboxymethyl)piperidin-4-yl)-2-(methylamino) propanoic acid
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KSDAFIIFFGFC

SEQ ID NO: 24 - protein, 12 aa, synthetic construct
  SITE 1: note = methyl-L-lysine
  SITE 2: note = methyl-L-Glutamine
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KQDQFTIFFGFC

SEQ ID NO: 25 - protein, 12 aa, synthetic construct
  SITE 1: note = methyl-L-lysine
  SITE 2: note = methyl-L-Glutamine
  SITE 4: note = 3-(1-(carboxymethyl)piperidin-4-yl)-2-(methylamino) propanoic acid
  SITE 5: note = 4-(2-amino-2-carboxyethyl)benzoic acid
  SITE 6: note = O-methyl threonine
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KQDAFTIFFGFC

SEQ ID NO: 26 - protein, 12 aa, synthetic construct
  SITE 1: note = methyl-L-Glutamine
  SITE 2: note = methyl-L-Glutamine
  SITE 4: note = methyl-L-lysine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDKFIIFFGFC

SEQ ID NO: 27 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 2-amino-3-(4-(2-aminoethoxy)phenyl)propanoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQXIIFFGFC

SEQ ID NO: 28 - protein, 12 aa, synthetic construct
  SITE 1: note = methyl-L-Glutamine
  SITE 2: note = methyl-L-Glutamine
  SITE 4: note = methyl-L-Glutamine
  SITE 5: note = 4-(2-amino-2-carboxyethyl)benzoic acid
  SITE 8: note = 2-amino-3-(4-(2-aminoethoxy)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
QQDQFIIXFGFC

SEQ ID NO: 29 - protein, 12 aa, synthetic construct
  SITE 1: note = methyl-L-lysine
  SITE 2: note = methyl-L-Glutamine
  SITE 3: note = 2,6-diamino-6-oxohexanoic acid
  SITE 4: note = methyl-L-Glutamine
  SITE 5: note = 4-(2-amino-2-carboxyethyl)benzoic acid
  SITE 6: note = O-methyl threonine
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KQQQFTIYFGFC

SEQ ID NO: 30 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFTIFFGXC

SEQ ID NO: 31 - protein, 13 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = methyl-L-Cysteine
  SITE 13: note = amidated D-Glutamic Acid
  SITE 1..12: note = Optionally cyclic
KIDQFTIFFGXCE

SEQ ID NO: 32 - protein, 13 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = methyl-L-Cysteine
  SITE 13: note = amidated Glutamic Acid
  SITE 1..12: note = Optionally cyclic
KIDQFTIFFGXCE

SEQ ID NO: 33 - protein, 12 aa, synthetic construct
  SITE 1: note = N6-(glutamyl)-N2-methyl-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 34 - protein, 12 aa, synthetic construct
  SITE 1: note = N6-( histidyl)-N2-methyl-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 35 - protein, 12 aa, synthetic construct
  SITE 1: note = methyl-L-lysine
  SITE 2: note = methyl-L-Glutamine
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KQDQFTIFFGXC

SEQ ID NO: 36 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(4-(1-methyl-1H-pyrazol-4-yl)phenyl)propa noic acid
  SITE 12: note = amididated Methyl-L-Cysteine
  SITE 1..12: note = Optionally cyclic
QSDQFTIFFGFC

SEQ ID NO: 37 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(2-(4-(carboxymethyl)piperazine-1-carb oxamido)ethoxy)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
QSDQFTIXFGXC

SEQ ID NO: 38 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-((4-(carboxymethyl)piperazine-1-carbox amido)methyl)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
QSDQFTIFFGXC

SEQ ID NO: 39 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-ureidophenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
QSDQFTIFFGXC

SEQ ID NO: 40 - protein, 12 aa, synthetic construct
  SITE 1: note = methyl-L-Glutamine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(2-(4-(carboxymethyl)piperazin-1-yl)-2 -oxoethoxy)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
QSDQFTIFFGXC

SEQ ID NO: 41 - protein, 12 aa, synthetic construct
  SITE 1: note = N6-(phenylalanyl)-N2-methyl-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 42 - protein, 12 aa, synthetic construct
  SITE 1: note = N6-(2-amino-4,4-dimethylpentanoyl)-N2-methyl-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 43 - protein, 12 aa, synthetic construct
  SITE 1: note = methyl-L-Alanine
  SITE 2: note = methyl-L-Glutamine
  SITE 4: note = methyl-L-Glutamine
  SITE 5: note = 4-(2-amino-2-carboxyethyl)benzoic acid
  SITE 8: note = 4-(2-amino-2-carboxyethyl)benzoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
AQDQFIIFFGXC

SEQ ID NO: 44 - protein, 12 aa, synthetic construct
  SITE 1: note = methyl-L-Alanine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
ASDQFTIXFGXC

SEQ ID NO: 45 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFTIYFGXC

SEQ ID NO: 46 - protein, 12 aa, synthetic construct
  SITE 1: note = methyl-L-lysine
  SITE 2: note = (2S)-6-{[4-(carboxymethyl)piperazine-1-carbonyl]ami no}-2-(methylamino)hexanoic acid
  SITE 4: note = 3-(4-(carboxymethyl)piperazine-1-carboxamido)-2-(me thylamino)propanoic acid
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KKDXFTIFFGXC

SEQ ID NO: 47 - protein, 12 aa, synthetic construct
  SITE 1: note = methyl-L-lysine
  SITE 2: note = (2S)-6-{[4-(carboxymethyl)piperazine-1-carbonyl]ami no}-2-(methylamino)hexanoic acid
  SITE 4: note = 3-(4-(carboxymethyl)piperazine-1-carboxamido)-2-(me thylamino)propanoic acid
  SITE 5: note = 2-amino-3-(6-((carboxymethyl)amino)pyridin-3-yl)pro panoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KKDXXTIFFGXC

SEQ ID NO: 48 - protein, 12 aa, synthetic construct
  SITE 1: note = methyl-L-lysine
  SITE 2: note = (2S)-6-{[4-(carboxymethyl)piperazine-1-carbonyl]ami no}-2-(methylamino)hexanoic acid
  SITE 4: note = (2S)-6-{[4-(carboxymethyl)piperazine-1-carbonyl]ami no}-2-(methylamino)hexanoic acid
  SITE 5: note = 4-(2-amino-2-carboxyethyl)benzoic acid
  SITE 6: note = O-methyl threonine
  SITE 8: note = 2-amino-3-(4-(2-(4-(carboxymethyl)piperazine-1-carb oxamido)ethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KKDKFTIXFGXC

SEQ ID NO: 49 - protein, 12 aa, synthetic construct
  SITE 1: note = methyl-L-lysine
  SITE 2: note = (2S)-6-{[4-(carboxymethyl)piperazine-1-carbonyl]ami no}-2-(methylamino)hexanoic acid
  SITE 4: note = (2S)-6-{[4-(carboxymethyl)piperazine-1-carbonyl]ami no}-2-(methylamino)hexanoic acid
  SITE 5: note = 4-(2-amino-2-carboxyethyl)benzoic acid
  SITE 6: note = O-methyl threonine
  SITE 8: note = 2-amino-3-(4-(2-(4-(carboxymethyl)piperazin-1-yl)-2 -oxoethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KKDKFTIFFGXC

SEQ ID NO: 50 - protein, 12 aa, synthetic construct
  SITE 1: note = 3-(4-(carboxymethyl)piperazine-1-carboxamido)-2-(me thylamino)propanoic acid
  SITE 2: note = 4-methoxy-2-(methylamino)butanoic acid
  SITE 4: note = 3-(4-(carboxymethyl)piperazine-1-carboxamido)-2-(me thylamino)propanoic acid
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
XSDXFTIXFGXC

SEQ ID NO: 51 - protein, 12 aa, synthetic construct
  SITE 1: note = 3-(4-(carboxymethyl)piperazine-1-carboxamido)-2-(me thylamino)propanoic acid
  SITE 2: note = 4-methoxy-2-(methylamino)butanoic acid
  SITE 4: note = 3-(4-(carboxymethyl)piperazine-1-carboxamido)-2-(me thylamino)propanoic acid
  SITE 5: note = 2-amino-3-(6-((carboxymethyl)amino)pyridin-3-yl)pro panoic acid
  SITE 6: note = O-methyl threonine
  SITE 8: note = 2-amino-3-(4-(2-aminoethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
XSDXXTIXFGXC

SEQ ID NO: 52 - protein, 12 aa, synthetic construct
  SITE 1: note = methyl-L-lysine
  SITE 2: note = 4-methoxy-2-(methylamino)butanoic acid
  SITE 4: note = 3-(4-(carboxymethyl)piperazine-1-carboxamido)-2-(me thylamino)propanoic acid
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDXFTIFFGXC

SEQ ID NO: 53 - protein, 12 aa, synthetic construct
  SITE 1: note = methyl-L-lysine
  SITE 2: note = 4-methoxy-2-(methylamino)butanoic acid
  SITE 4: note = 3-(4-(carboxymethyl)piperazine-1-carboxamido)-2-(me thylamino)propanoic acid
  SITE 5: note = 2-amino-3-(6-((carboxymethyl)amino)pyridin-3-yl)pro panoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDXXTIFFGXC

SEQ ID NO: 54 - protein, 12 aa, synthetic construct
  SITE 1: note = 3-(4-(carboxymethyl)piperazine-1-carboxamido)-2-(me thylamino)propanoic acid
  SITE 2: note = 4-methoxy-2-(methylamino)butanoic acid
  SITE 4: note = 3-(4-(carboxymethyl)piperazine-1-carboxamido)-2-(me thylamino)propanoic acid
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
XSDXFTIXFGXC

SEQ ID NO: 55 - protein, 12 aa, synthetic construct
  SITE 1: note = MeKCOpipzaa
  SITE 2: note = 4-methoxy-2-(methylamino)butanoic acid
  SITE 4: note = MeDapCOpipzaa
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDXFTIXFGXC

SEQ ID NO: 56 - protein, 12 aa, synthetic construct
  SITE 1: note = (2S)-6-{[4-(carboxymethyl)piperazine-1-carbonyl]ami no}-2-(methylamino)hexanoic acid
  SITE 2: note = methyl-L-Alanine
  SITE 4: note = methyl-L-Tyrosine
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = methyl-L-Tyrosine
  SITE 11: note = 2-amino-3-(6-phenylpyridin-3-yl)propanoic acid
  SITE 12: note = amidated methyl-L-Cysteine
  SITE 1..12: note = Optionally cyclic
KADYFTIXYGXC

SEQ ID NO: 57 - protein, 12 aa, synthetic construct
  SITE 1: note = (2S)-6-{[4-(carboxymethyl)piperazine-1-carbonyl]ami no}-2-(methylamino)hexanoic acid
  SITE 2: note = methyl-L-Alanine
  SITE 3: note = Methyl-L-Tyrosine
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid
  SITE 9: note = Methyl-L-Tyrosine
  SITE 11: note = 2-amino-3-(6-phenylpyridin-3-yl)propanoic acid
  SITE 12: note = amidated methyl-L-Cysteine
  SITE 1..12: note = Optionally cyclic
KAYDFTIXYGXC

SEQ ID NO: 58 - protein, 12 aa, synthetic construct
  SITE 1: note = (2S)-6-{[4-(carboxymethyl)piperazine-1-carbonyl]ami no}-2-(methylamino)hexanoic acid
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFTIYFGXC

SEQ ID NO: 59 - protein, 12 aa, synthetic construct
  SITE 1: note = (2S)-6-{[4-(carboxymethyl)piperazine-1-carbonyl]ami no}-2-(methylamino)hexanoic acid
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = (3R)-2-amino-3-hydroxypentanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFXIYFGXC

SEQ ID NO: 60 - protein, 13 aa, synthetic construct
  SITE 1: note = acetylated methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = methyl-L-Cysteine
  SITE 1..12: note = Optionally cyclic
  SITE 13: note = amidated Glycine(PEG10c)Lysine
KIDQFIIYFGXCG

SEQ ID NO: 61 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFIIFFGXC

SEQ ID NO: 62 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFTIYFGXC

SEQ ID NO: 63 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFTIFFGXC

SEQ ID NO: 64 - protein, 12 aa, synthetic construct
  SITE 1: note = methyl-L-lysine
  SITE 2: note = methyl-L-Isoleucine
  SITE 4: note = methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 65 - protein, 12 aa, synthetic construct
  SITE 1: note = methyl-L-lysine
  SITE 2: note = methyl-L-Glutamine
  SITE 3: note = 2,6-diamino-6-oxohexanoic acid
  SITE 4: note = methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated methyl-L-Cysteine
  SITE 1..12: note = Optionally cyclic
KQQQFTIFFGFC

SEQ ID NO: 66 - protein, 11 aa, synthetic construct
  SITE 1: note = methyl-L-Alanine
  SITE 2: note = Methyl-L-Tyrosine
  SITE 5: note = O-methyl threonine
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
AYDYTIYFGXC

SEQ ID NO: 67 - protein, 11 aa, synthetic construct
  SITE 1: note = methyl-L-Alanine
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
ASDYIIYFGXC

SEQ ID NO: 68 - protein, 11 aa, synthetic construct
  SITE 1: note = methyl-L-Alanine
  SITE 3: note = 2-amino-3-(1H-pyrrolo[3,2-c]pyridin-3-yl)propanoic acid
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
ASWYIIYFGXC

SEQ ID NO: 69 - protein, 11 aa, synthetic construct
  SITE 1: note = methyl-L-Alanine
  SITE 2: note = 2-(methylamino)-3-(pyridin-3-yl)propanoic acid
  SITE 5: note = (3R)-2-amino-3-methylpentanoic acid
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
AFDYTIYFGXC

SEQ ID NO: 70 - protein, 11 aa, synthetic construct
  SITE 1: note = methyl-L-Alanine
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
ASQYIIYFGXC

SEQ ID NO: 71 - protein, 11 aa, synthetic construct
  SITE 1: note = methyl-L-Alanine
  SITE 2: note = O-methyl threonine
  SITE 5: note = 1-aminocyclopentane-1-carboxylic acid
  SITE 8: note = 2-(methylamino)-3-(naphthalen-2-yl)propanoic acid
  SITE 10: note = 2-amino-3-(6-phenylpyridin-3-yl)propanoic acid
  SITE 11: note = amidated methyl-L-Cysteine
  SITE 1..11: note = Optionally cyclic
ATDYXIYFGXC

SEQ ID NO: 72 - protein, 12 aa, synthetic construct
  SITE 1: note = methyl-L-lysine
  SITE 2: note = 4-methoxy-2-(methylamino)butanoic acid
  SITE 4: note = methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KSDQFIIYFGXC

SEQ ID NO: 73-239 - skipped (167 entries)

SEQ ID NO: 240 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Isoleucine
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFIIYFGXC

SEQ ID NO: 241 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 4-(2-amino-2-carboxyethyl)benzoic acid
  SITE 12: note = amidated Methyl-L-Cysteine
  SITE 1..12: note = Optionally cyclic
KSDQFIIFFGFC

SEQ ID NO: 242 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Optionally cyclic
KSDQFIIFFGFC

SEQ ID NO: 243 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Glutamine
  SITE 4: note = Methyl-L-Glutamine
  SITE 5: note = 4-(2-amino-2-carboxyethyl)benzoic acid
  SITE 6: note = (3R)-2-amino-3-methoxybutanoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Optionally cyclic
KQDQFTIFFGFC

SEQ ID NO: 244 - protein, 12 aa, synthetic construct
  SITE 1: note = Methyl-L-Glutamine
  SITE 2: note = Methyl-L-Glutamine
  SITE 4: note = Methyl-L-Glutamine
  SITE 5: note = 4-(2-amino-2-carboxyethyl)benzoic acid
  SITE 8: note = 2-amino-3-(4-(2-aminoethoxy)phenyl)propanoic acid(2,2',2'',2'''-(1,4,7,10-Tetraazacyclododecane-1,4,7,1 0-tetrayl)tetraacetic acid-Lutetium)
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Optionally cyclic
QQDQFIIXFGFC

SEQ ID NO: 245 - protein, 12 aa, synthetic construct
  SITE 1: note = Methyl-L-Glutamine
  SITE 2: note = Methyl-L-Glutamine
  SITE 4: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Optionally cyclic
QQDKFIIFFGFC

SEQ ID NO: 246 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFTIFFGXC

SEQ ID NO: 247 - protein, 13 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 13: note = D-Glutamic acid
  SITE 1..12: note = Optionally cyclic
KIDQFTIFFGXCE

SEQ ID NO: 248 - protein, 13 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFTIFFGXCE

SEQ ID NO: 249 - protein, 12 aa, synthetic construct
  SITE 1: note = N6-(glutamyl)-N2-methyl-lysine(2,2',2'',2'''-(1,4,7 ,10-Tetraazacyclododecane-1,4,7,10-tetrayl)tetraacetic acid-Lutetium)
  SITE 2: note = methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFIIFFGXC

SEQ ID NO: 250 - protein, 12 aa, synthetic construct
  SITE 1: note = N6-( histidyl)-N2-methyl-lysine(2,2',2'',2'''-(1,4, 7,10-Tetraazacyclododecane-1,4,7,10-tetrayl)tetraacetic acid-Lutetium)
  SITE 2: note = methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFIIFFGXC

SEQ ID NO: 251 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Glutamine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KQDQFTIFFGXC

SEQ ID NO: 252 - protein, 12 aa, synthetic construct
  SITE 1: note = Methyl-L-Alanine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid(2,2',2'',2'''-(1,4,7,10-Tetraazacyclododecane-1,4,7,1 0-tetrayl)tetraacetic acid-Lutetium)
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
ASDQFTIXFGXC

SEQ ID NO: 253 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KSDQFTIYFGXC

SEQ ID NO: 254 - protein, 12 aa, synthetic construct
  SITE 1: note = N6-(phenylalanyl)-N2-methyl-lysine(2,2',2'',2'''-(1 ,4,7,10-Tetraazacyclododecane-1,4,7,10-tetrayl)tetraacetic acid-Lutetium)
  SITE 2: note = methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFIIFFGXC

SEQ ID NO: 255 - protein, 12 aa, synthetic construct
  SITE 1: note = N6-(2-amino-4,4-dimethylpentanoyl)-N2-methyl-lysine (2,2',2'',2'''-(1,4,7,10-Tetraazacyclododecane-1,4,7,10-te trayl)tetraacetic acid-Lutetium)
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFIIFFGXC

SEQ ID NO: 256 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated methyl-L-Cysteine
  SITE 1..12: note = Optionally cyclic
KIDQFIIFFGXC

SEQ ID NO: 257 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFTIYFGXC

SEQ ID NO: 258 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KSDQFIIYFGXC

SEQ ID NO: 259 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid - Lutetium
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KSDQFTIFFGXC

SEQ ID NO: 260-353 - skipped (94 entries)

SEQ ID NO: 354 - protein, 12 aa, synthetic construct
  SITE 1: note = N6-(tert-butoxycarbonyl)-N2-methyllysine
  SITE 2: note = 4-methoxy-2-(methylamino)butanoic acid
  SITE 3: note = 2-amino-4-(tert-butoxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = 2-amino-3-(4-(tert-butoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated MeC(Trt)
KSDQFIIYFGXC

SEQ ID NO: 355 - protein, 12 aa, synthetic construct
  SITE 1: note = N2-(2-bromoacetyl)-N6-(tert-butoxycarbonyl)-N2-meth yllysine
  SITE 2: note = 4-methoxy-2-(methylamino)butanoic acid
  SITE 3: note = 2-amino-4-(tert-butoxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = 2-amino-3-(4-(tert-butoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated MeC(Trt)
KSDQFIIYFGXC

SEQ ID NO: 356 - protein, 12 aa, synthetic construct
  SITE 1: note = N2-(2-bromoacetyl)-N6-(tert-butoxycarbonyl)-N2-meth yllysine
  SITE 2: note = 4-methoxy-2-(methylamino)butanoic acid
  SITE 3: note = 2-amino-4-(tert-butoxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = 2-amino-3-(4-(tert-butoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated S-tritylcysteine
KSDQFIIYFGXC

SEQ ID NO: 357 - protein, 12 aa, synthetic construct
  SITE 1: note = N2-(2-bromoacetyl)-N2-methyllysine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
KSDQFIIYFGXC

SEQ ID NO: 358 - protein, 12 aa, synthetic construct
  SITE 1: note = Methyl-L-Lysine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFIIYFGXC

SEQ ID NO: 359 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFIIYFGXC

SEQ ID NO: 360 - protein, 12 aa, synthetic construct
  SITE 1: note = N2-(2-bromoacetyl)-N6-(tert-butoxycarbonyl)-N2-meth yllysine
  SITE 2: note = 4-methoxy-2-(methylamino)butanoic acid
  SITE 3: note = 2-amino-4-(tert-butoxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated MeC(Trt)
KSDQFTIYFGXC

SEQ ID NO: 361 - protein, 12 aa, synthetic construct
  SITE 1: note = N2-(2-bromoacetyl)-N6-(tert-butoxycarbonyl)-N2-meth yllysine
  SITE 2: note = 4-methoxy-2-(methylamino)butanoic acid
  SITE 3: note = 2-amino-4-(tert-butoxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 6: note = O-methyl threonine
  SITE 8: note = 2-amino-3-(4-(tert-butoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated S-tritylcysteine
KSDQFTIYFGXC

SEQ ID NO: 362 - protein, 12 aa, synthetic construct
  SITE 1: note = N2-(2-bromoacetyl)-N2-methyllysine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
KSDQFTIYFGXC

SEQ ID NO: 363 - protein, 12 aa, synthetic construct
  SITE 1: note = Methyl-L-Lysine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFTIYFGXC

SEQ ID NO: 364 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 12: note = Cyclic
KIDQFIIYFGXC

SEQ ID NO: 365 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFIIFFGXC

SEQ ID NO: 366 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Cyclic
KSDQFIIFFGFC

SEQ ID NO: 367 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Glutamine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Cyclic
KQDQFTIFFGFC

SEQ ID NO: 368 - protein, 12 aa, synthetic construct
  SITE 1: note = Methyl-L-Glutamine
  SITE 2: note = Methyl-L-Glutamine
  SITE 4: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 5: note = 4-(2-amino-2-carboxyethyl)benzoic acid
  SITE 8: note = 2-amino-3-(4-(carboxymethyl)phenyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Cyclic
QQDKFIIFFGFC

SEQ ID NO: 369 - protein, 13 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 13: note = de
  SITE 1..12: note = Cyclic
KIDQFTIFFGXCE

SEQ ID NO: 370 - protein, 13 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIFFGXCE

SEQ ID NO: 371 - protein, 12 aa, synthetic construct
  SITE 1: note = N6-(histidyl)-N2-methyl-lysine(2,2',2'',2'''-(1,4,7 ,10-Tetraazacyclododecane-1,4,7,10-tetrayl)tetraacetic acid)
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 372 - protein, 12 aa, synthetic construct
  SITE 1: note = N6-(phenylalanyl)-N2-methyl-lysine(2,2',2'',2'''-(1 ,4,7,10-Tetraazacyclododecane-1,4,7,10-tetrayl)tetraacetic acid)
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 373 - protein, 12 aa, synthetic construct
  SITE 1: note = N6-(2-amino-4,4-dimethylpentanoyl)-N2-methyl-lysine (2,2',2'',2'''-(1,4,7,10-Tetraazacyclododecane-1,4,7,10-te trayl)tetraacetic acid)
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 374 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIYFGXC

SEQ ID NO: 375 - protein, 12 aa, synthetic construct
  SITE 1: note = Methyl-L-Glutamine
  SITE 2: note = Methyl-L-Glutamine
  SITE 4: note = Methyl-L-Glutamine
  SITE 5: note = 4-(2-amino-2-carboxyethyl)benzoic acid
  SITE 8: note = 2-amino-3-(4-(2-aminoethoxy)phenyl)propanoic acid(2,2',2'',2'''-(1,4,7,10-Tetraazacyclododecane-1,4,7,1 0-tetrayl)tetraacetic acid)
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Cyclic
QQDQFIIXFGFC

SEQ ID NO: 376 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIFFGXC

SEQ ID NO: 377 - protein, 12 aa, synthetic construct
  SITE 1: note = N6-(glutamyl)-N2-methyl-lysine(2,2',2'',2'''-(1,4,7 ,10-Tetraazacyclododecane-1,4,7,10-tetrayl)tetraacetic acid)
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 378 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 379 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIYFGXC

SEQ ID NO: 380 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFTIFFGXC

SEQ ID NO: 381 - protein, 12 aa, synthetic construct
  SITE 1: note = 2,2',2''-(10-(2-((5-carboxy-5-(methylamino)pentyl)a mino)-2-oxoethyl)-1,4,7,10-tetraazacyclododecane-1,4,7-tri yl)triacetic acid
  SITE 2: note = Methyl-L-Glutamine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KQDQFTIFFGXC

SEQ ID NO: 382 - protein, 12 aa, synthetic construct
  SITE 1: note = methyl-L-Alanine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(2-aminoethoxy)phenyl)propanoic acid(2,2',2'',2'''-(1,4,7,10-Tetraazacyclododecane-1,4,7,1 0-tetrayl)tetraacetic acid)
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
ASDQFTIXFGXC

SEQ ID NO: 383 - protein, 12 aa, synthetic construct
  SITE 1: note = N2-((allyloxy)carbonyl)-N2-methyllysine
  SITE 2: note = Methyl-L-Isoleucine
  SITE 3: note = 2-amino-4-((3-methylpentan-3-yl)oxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = Tyrosine(O-(2-tert-butoxy-2-oxoethyl)
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = S-tritylcysteine
KIDQFIIYFGXC

SEQ ID NO: 384 - protein, 12 aa, synthetic construct
  SITE 1: note = 2-((((9H-fluoren-9-yl)methoxy)carbonyl)amino)-4,4-d imethylpentanoic acid-N2-((allyloxy)carbonyl)-N2-methyllysine
  SITE 2: note = Methyl-L-Isoleucine
  SITE 3: note = 2-amino-4-((3-methylpentan-3-yl)oxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = Tyrosine(O-(2-tert-butoxy-2-oxoethyl)
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated S-tritylcysteine
KIDQFIIYFGXC

SEQ ID NO: 385 - protein, 12 aa, synthetic construct
  SITE 1: note = 2-((((9H-fluoren-9-yl)methoxy)carbonyl)amino)-4,4-d imethylpentanoic acid-Methyl-L-Lysine
  SITE 2: note = Methyl-L-Isoleucine
  SITE 3: note = 2-amino-4-((3-methylpentan-3-yl)oxy)-4-oxobutanoic acid
  SITE 4: note = S-tritylcysteine
  SITE 5: note = 2-amino-3-(4-(tert-butoxycarbonyl)phenyl)propanoic acid
  SITE 8: note = Tyrosine(O-(2-tert-butoxy-2-oxoethyl)
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated S-tritylcysteine
KIDQFIIYFGXC

SEQ ID NO: 386 - protein, 12 aa, synthetic construct
  SITE 1: note = 2-((((9H-fluoren-9-yl)methoxy)carbonyl)amino)-4,4-d imethylpentanoic acid-N2-(2-chloroacetyl)-N6-(tert-butoxyc arbonyl)-N2-methyllysine
  SITE 2: note = Methyl-L-Isoleucine
  SITE 3: note = 2-amino-4-((3-methylpentan-3-yl)oxy)-4-oxobutanoic acid
  SITE 4: note = N2-methyl-N5-tritylglutamine
  SITE 5: note = 2-amino-3-(4-(tert-butoxycarbonyl)phenyl)propanoic acid
  SITE 8: note = Tyrosine(O-(2-tert-butoxy-2-oxoethyl)
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated S-tritylcysteine
KIDQFIIYFGXC

SEQ ID NO: 387 - protein, 12 aa, synthetic construct
  SITE 1: note = 2-((((9H-fluoren-9-yl)methoxy)carbonyl)amino)-4,4-d imethylpentanoic acid-N2-(2-chloroacetyl)-N6-(tert-butoxyc arbonyl)-N2-methyllysine
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
KIDQFIIFFGXC

SEQ ID NO: 388 - protein, 12 aa, synthetic construct
  SITE 1: note = 2-((((9H-fluoren-9-yl)methoxy)carbonyl)amino)-4,4-d imethylpentanoic acid-methyl-L-Lysine
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 389 - protein, 12 aa, synthetic construct
  SITE 1: note = N6-(2-amino-4,4-dimethylpentanoyl)-N2-methyllysine
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGXC

SEQ ID NO: 390 - protein, 12 aa, synthetic construct
  SITE 1: note = 51-(methylamino)-5,45-dioxo-1-((3aS,4S,6aR)-2-oxohe xahydro-1H-thieno[3,4-d]imidazol-4-yl)-9,12,15,18,21,24,27 ,30,33,36,39,42-dodecaoxa-6,46-diazadopentacontan-52-oic acid
  SITE 2: note = Methyl-L-Isoleucine
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIYFGXC

SEQ ID NO: 391 - protein, 12 aa, synthetic construct
  SITE 1: note = 51-(methylamino)-5,45-dioxo-1-((3aS,4S,6aR)-2-oxohe xahydro-1H-thieno[3,4-d]imidazol-4-yl)-9,12,15,18,21,24,27 ,30,33,36,39,42-dodecaoxa-6,46-diazadopentacontan-52-oic acid
  SITE 2: note = Methyl-L-Glutamine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KQDQFTIFFGXC

SEQ ID NO: 392 - protein, 12 aa, synthetic construct
  SITE 1: note = Methyl-L-Glutamine
  SITE 2: note = Methyl-L-Glutamine
  SITE 4: note = Methyl-L-Glutamine
  SITE 5: note = 4-(2-amino-2-carboxyethyl)benzoic acid
  SITE 8: note = 2-amino-3-(4-((4,44-dioxo-48-((3aS,4S,6aR)-2-oxohex ahydro-1H-thieno[3,4-d]imidazol-4-yl)-7,10,13,16,19,22,25, 28,31,34,37,40-dodecaoxa-3,43-diazaoctatetracontyl)oxy)phe nyl)propanoic acid
  SITE 9: note = 2-(methylamino)-3-(naphthalen-2-yl)propanoic acid
  SITE 11: note = 2-amino-3-(4-(1-methyl-1H-pyrazol-4-yl)phenyl)propa noic acid
  SITE 12: note = amidated Methyl-L-Cysteine
  SITE 1..12: note = Cyclic
QQDQFIIXFGFC

SEQ ID NO: 393 - protein, 12 aa, synthetic construct
  SITE 1: note = 51-(methylamino)-5,45-dioxo-1-((3aS,4S,6aR)-2-oxohe xahydro-1H-thieno[3,4-d]imidazol-4-yl)-9,12,15,18,21,24,27 ,30,33,36,39,42-dodecaoxa-6,46-diazadopentacontan-52-oic acid
  SITE 2: note = Methyl-L-Isoleucine
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIFFGFC

SEQ ID NO: 394 - protein, 12 aa, synthetic construct
  SITE 1: note = 51-(methylamino)-5,45-dioxo-1-((3aS,4S,6aR)-2-oxohe xahydro-1H-thieno[3,4-d]imidazol-4-yl)-9,12,15,18,21,24,27 ,30,33,36,39,42-dodecaoxa-6,46-diazadopentacontan-52-oic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIFFGXC

SEQ ID NO: 395 - protein, 12 aa, synthetic construct
  SITE 1: note = 2-((1E,3E)-5-((Z)-1-(6-((5-carboxy-5-(methylamino)p entyl)amino)-6-oxohexyl)-3,3-dimethyl-5-sulfoindolin-2-yli dene)penta-1,3-dien-1-yl)-1-ethyl-3,3-dimethyl-3H-indol-1- ium-5-sulfonate
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KSDQFTIYFGXC

SEQ ID NO: 396 - protein, 12 aa, synthetic construct
  SITE 1: note = 2-((1E,3E)-5-((Z)-1-(6-((5-carboxy-5-(methylamino)p entyl)amino)-6-oxohexyl)-3,3-dimethyl-5-sulfoindolin-2-yli dene)penta-1,3-dien-1-yl)-1-ethyl-3,3-dimethyl-3H-indol-1- ium-5-sulfonate
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIFFGXC

SEQ ID NO: 397 - protein, 12 aa, synthetic construct
  SITE 1: note = 2-((1E,3E)-5-((Z)-1-(6-((5-carboxy-5-(methylamino)p entyl)amino)-6-oxohexyl)-3,3-dimethyl-5-sulfoindolin-2-yli dene)penta-1,3-dien-1-yl)-1-ethyl-3,3-dimethyl-3H-indol-1- ium-5-sulfonate
  SITE 2: note = Methyl-L-Isoleucine
  SITE 4: note = Methyl-L-Glutamine
  SITE 5: note = 4-(2-amino-2-carboxyethyl)benzoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFIIYFGXC

SEQ ID NO: 398 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 399 - protein, 13 aa, synthetic construct
  SITE 1: note = fluorine-18 Lysine
KYNDRLPLYISNP

SEQ ID NO: 400 - protein, 5 aa, synthetic construct
EAAAK

SEQ ID NO: 401 - protein, 30 aa, synthetic construct
  VARIANT 6..30: note = GGGGS repeats may be deleted
GGGGSGGGGSGGGGSGGGGSGGGGSGGGGS

SEQ ID NO: 402 - protein, 12 aa, synthetic construct
  SITE 1: note = methyl-L-Alanine
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-[(1E,3E)-5-[(2E)-1-{5-[(2-{4- [(2S) -2-amino-2-ca rboxyethyl]phenoxy}ethyl)carbamoyl]pentyl}-3,3-dimethyl-5- sulfo-2,3-dihydro-1H-indol-2-ylidene]penta-1,3-dien-1-yl] -1-ethyl-3,3-dimethyl-3H-indol-1-ium-5-sulfonate
  SITE 9: note = 2-(methylamino)-3-(naphthalen-2-yl)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
ASDQFTIXFGXC

SEQ ID NO: 403 - protein, 12 aa, synthetic construct
  SITE 1: note = 2-[(1E,3E)-5-[(2E)-1-(5-{[(5S)-5-carboxy-5-(methyla mino)pentyl]carbamoyl}pentyl)-3,3-dimethyl-5-sulfo-2,3-dih ydro-1H-indol-2-ylidene]penta-1,3-dien-1-yl]-1-ethyl-3,3-d imethyl-3H-indol-1-ium-5-sulfonate
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KSDQFIIFFGXC

SEQ ID NO: 404 - protein, 12 aa, synthetic construct
  SITE 1: note = 2-[(1E,3E)-5-[(2E)-1-(5-{[(5S)-5-carboxy-5-(methyla mino)pentyl]carbamoyl}pentyl)-3,3-dimethyl-5-sulfo-2,3-dih ydro-1H-indol-2-ylidene]penta-1,3-dien-1-yl]-1-ethyl-3,3-d imethyl-3H-indol-1-ium-5-sulfonate
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KIDQFTIYFGXC

SEQ ID NO: 405 - protein, 12 aa, synthetic construct
  SITE 1: note = 2-[(1E,3E)-5-[(2E)-1-(5-{[(5S)-5-carboxy-5-(methyla mino)pentyl]carbamoyl}pentyl)-3,3-dimethyl-5-sulfo-2,3-dih ydro-1H-indol-2-ylidene]penta-1,3-dien-1-yl]-1-ethyl-3,3-d imethyl-3H-indol-1-ium-5-sulfonate
  SITE 2: note = 4-methoxy-2-(methylamino)butanoic acid
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Optionally cyclic
KSDQFTIFFGXC

SEQ ID NO: 406 - protein, 12 aa, synthetic construct
  SITE 1: note = 2,2'-(7-(2-((5-carboxy-5-(methylamino)pentyl)amino) -2-oxoethyl)-1,4,7-triazonane-1,4-diyl)diacetic acid
  SITE 2: note = Methyl-L-Isoleucine
  SITE 4: note = Methyl-L-Glutamine
  SITE 5: note = 4-(2-amino-2-carboxyethyl)benzoic acid
  SITE 6: note = O-methyl threonine
  SITE 8: note = 2-amino-3-(4-(carboxymethoxy)phenyl)propanoic acid
  SITE 9: note = 3-(isoquinolin-7-yl)-2-(methylamino)propanoic acid
  SITE 11: note = 2-amino-3-(6-phenylpyridin-3-yl)propanoic acid
  SITE 12: note = amidated Methyl-L-Cysteine
  SITE 1..12: note = Cyclic
KIDQFTIFFGXC